(19) United States
(12) Patent Application Publication
(10) Pub. No.: US 2025/0368996 A1
(43) Pub. Date: Dec. 4, 2025

(54) MODIFIED ANTISENSE OLIGONUCLEOTIDES FOR TREATING HEPATITIS B VIRUS

(71) Applicant: Aligos Therapeutics, Inc., South San Francisco, CA (US)

(72) Inventors: Vivek K. Rajwanshi, Cupertino, CA (US); Jin Hong, Pacifico, CA (US); Leonid Beigelman, San Mateo, CA (US); Antitsa Stoycheva, South San Francisco, CA (US); Aneerban Bhattacharya, South San Francisco, CA (US); David B. Smith, South San Francisco, CA (US); Kellan Passow, South San Francisco, CA (US); Rostom Ahmed-Belkacem, South San Francisco, CA (US); Jacquelyn Sousa, South San Francisco, CA (US)

(73) Assignee: Aligos Therapeutics, Inc., South San Francisco, CA (US)

(21) Appl. No.: 19/206,988

(22) Filed: May 13, 2025

Related U.S. Application Data
(60) Provisional application No. 63/647,238, filed on May 14, 2024.
(60) Provisional application No. 63/727,158, filed on Dec. 2, 2024.

(51) Int. Cl. C12N 15/113; A61P 31/14

(52) U.S. Cl. CPC C12N 15/1131; A61P 31/14; C12N 2310/11; C12N 2310/315; C12N 2310/321; C12N 2310/3231; C12N 2310/341; C12N 2310/351; C12N 2320/32

(57) ABSTRACT

Provided herein are antisense oligonucleotides and compositions that include the disclosed ASOs. The disclosed ASOs and compositions can be used for treating hepatitis B.

### CROSS REFERENCE TO RELATED APPLICATIONS

This application claims priority to and the benefit of U.S. Provisional Application No. 63/647,238, filed May 14, 2024, and U.S. Provisional Application No. 63/727,158, filed Dec. 2, 2024. The contents of these applications are incorporated herein by reference in their entireties.

### SEQUENCE LISTING

The instant application contains a Sequence Listing which has been submitted electronically in XML format and is hereby incorporated by reference in its entirety. Said XML copy, created on Aug. 15, 2025, is named 122400-0435_SL.xml and is 17,053,529 bytes in size.

### TECHNICAL FIELD

This disclosure relates to antisense oligonucleotides (ASOs) comprising modified nucleotides, compositions, and uses thereof. More particularly, this disclosure relates to ASOs, pharmaceutical compositions, and uses thereof to treat diseases and infections, such as hepatitis B viral infection.

### BACKGROUND

The following description of the background of the present technology is provided simply as an aid in understanding the present technology and is not admitted to describe or constitute prior art to the present technology.

Around 300 million people are chronically infected with hepatitis B virus (HBV) worldwide. For these chronic hepatitis B (CHB) patients, HBsAg loss, a key aspect of “functional cures”, is the goal of many new therapies being developed. Antisense oligonucleotides (ASOs) have been demonstrated to be an effective modality in reducing HBsAg in animal models, and in clinical studies through degradation of viral RNA and possibly through activation of the innate immune system.

However, treatment of HBV with antisense oligonucleotides still exhibits some safety problems, including liver toxicity. Thus, there is a need in the art to discover antisense oligonucleotides that have improved safety profiles and increased efficacy.

### SUMMARY

The present disclosure provides antisense oligonucleotide and compositions containing ASOs, as well as methods and uses for preventing or treating hepatitis B with the disclosed ASOs and compositions.

Disclosed herein is an antisense oligonucleotide (ASO) that is complementary to at least 5, 6, 7, 8, 9, 10, 11, 12, 13, 14, or 15 contiguous nucleotides within positions 1570-1610 of SEQ ID NO: 1, has a nucleic acid sequence comprising or consisting of 18-23 nucleotides and, optionally, at least one of the nucleotides is replaced with an abasic monomer, and comprises at least one phosphorothioate linkage and at least one 2′-O-methoxyethyl nucleotide.

In some embodiments, the ASO is complementary to at least 5, 6, 7, 8, 9, 10, 11, 12, 13, 14, or 15 contiguous nucleotides within positions 1575-1610 of SEQ ID NO: 1. In some embodiments, the ASO is complementary to at least 5, 6, 7, 8, 9, 10, 11, 12, 13, 14, or 15 contiguous nucleotides within positions 1579-1606 of SEQ ID NO: 1. In some embodiments, the nucleic acid sequence comprises or consists of any one of SEQ ID NOs: 321-352.

In some embodiments, the ASO comprises at least one 5-methylcytosine. In some embodiments, the ASO comprises two or three 5-methylcytosines.

In some embodiments, the at least one phosphorothioate linkage is a stereo-defined phosphorothioate linkage. In some embodiments, the ASO comprises (a) a 5′-wing region (A′) comprising 2 to 7 locked nucleotides or substituted nucleotides; (b) a central region (B′) comprising 5 or more contiguous nucleotides; and (c) a 3′-wing region (C′) comprising 2 to 7 locked nucleotides or substituted nucleotides.

In some embodiments, the central region (B′) comprises DNA nucleotides. In some embodiments, (i) the 5′-wing region (A′) comprises 1 to 7 phosphorothioate-linked locked nucleotides, (ii) the 3′-wing region (C′) comprises 1 to 7 phosphorothioate-linked locked nucleotides, or (iii) any combination thereof. In some embodiments, each locked nucleotide is independently selected from LNA, ScpBNA, AmNA, AmNA (N-Me), GuNA, GuNA (N—R), and any combination thereof. In some embodiments, the ASO comprises (a) a 5′-wing region (A′) comprising 5 nucleotides; (b) a central region (B′) comprising 10 nucleotides; and (c) a 3′-wing region (C′) comprising 5 nucleotides.

In some embodiments, (i) the 5′-wing region (A′) comprises a 2′-O-cyclopropyl nucleotide or a 2′-O-methylcyclopropyl nucleotide, (ii) the 3′-wing region (C′) comprises a 2′-O-cyclopropyl nucleotide or a 2′-O-methylcyclopropyl nucleotide, or (iii) any combination thereof. In some embodiments, (i) the 5′-wing region (A′) comprises at least one 2′-OMe nucleotide, (ii) the 3′-wing region (C′) comprises at least one 2′-OMe nucleotide, or (iii) any combination thereof.

In some embodiments, the central region (B′) comprises at least one RNA, at least one 2′-substituted nucleotide, at least one nucleotide with a modified base, at least one abasic monomer, or any combination thereof. In some embodiments, the at least one RNA, at least one substituted nucleotide, or at least one nucleotide with a modified base, or at least one abasic monomer is located at any one of positions 1-6 of the central region (B′) relative to the 5′ end of the ASO. In some embodiments, the substituted nucleotide is selected from 2′-O-cyp, 2′-O-mcyp, 2′-OMe, and 2′-OMe-3′-xylo. In some embodiments, the abasic monomer is selected from abasic monomer 1, abasic monomer 2, abasic monomer 3, and abasic monomer 4. In some embodiments, the nucleotide with a modified base is selected from (8nh)G, (8nh)A, (2s)T, and (5oh)C.

In some embodiments, the ASO further comprises 1-6 ribonucleotides attached to the 5′ end of the ASO or 1-3 ribonucleotides attached to the 3′ end of the ASO. In some embodiments, 4-6 ribonucleotides are attached to the 5′ end of the ASO.

Disclosed herein is an antisense oligonucleotide (ASO) that is complementary to at least 5, 6, 7, 8, 9, 10, 11, 12, 13, 14, or 15 contiguous nucleotides within positions 1570-1610 of SEQ ID NO: 1, has a nucleic acid sequence comprising or consisting of 18-23 nucleotides and, optionally, at least one of the nucleotides is replaced with an abasic monomer, and comprises 4-6 ribonucleotides attached to the 5′ end of the ASO or 1-3 ribonucleotides attached to the 3′ end of the ASO.

Disclosed herein is an antisense oligonucleotide (ASO) comprising or consisting of any one of SEQ ID NO: 3-320, 353-404, and 446-1344. ASOs of particular interest, due to observed activity, include but are not limited to, ASO-676 (SEQ ID NO: 697), ASO-677 (SEQ ID NO: 698), ASO-1037 (SEQ ID NO: 1058), ASO-707 (SEQ ID NO: 728), ASO-1192 (SEQ ID NO: 1213), ASO-651 (SEQ ID NO: 672), ASO-1166 (SEQ ID NO: 1187), ASO-1181 (SEQ ID NO: 1202), ASO-1179 (SEQ ID NO: 1200), and ASO-962 (SEQ ID NO: 983). In some embodiments, the ASO is selected from ASO-676 (SEQ ID NO: 697), ASO-677 (SEQ ID NO: 698), ASO-1037 (SEQ ID NO: 1058), ASO-707 (SEQ ID NO: 728), and ASO-1192 (SEQ ID NO: 1213). In some embodiments, the ASO is selected from ASO-651 (SEQ ID NO: 672), ASO-1166 (SEQ ID NO: 1187), ASO-1181 (SEQ ID NO: 1202), and ASO-1179 (SEQ ID NO: 1200). In some embodiments, the ASO is ASO-962 (SEQ ID NO: 983).

In some embodiments of any of the ASO disclosed herein, the ASO may further comprise a conjugate (e.g., a GalNAc) attached to the 5′ end or the 3′ end of the ASO or both the 5′ end and the 3′ end.

Disclosed herein is a pharmaceutical composition comprising the ASO according to any of the above aspects or embodiments and a pharmaceutically acceptable excipient.

Disclosed herein is a method of treating a subject having a Hepatitis B virus (HBV) infection, comprising administering to the subject with HBV an ASO according to any one of the aspects and embodiments described above or the pharmaceutical composition described above. In some embodiments, the method further comprises administering an additional therapeutic agent. In some embodiments, the additional treatment agent is selected from a nucleotide analog, nucleoside analog, a capsid assembly modulator (CAM), a recombinant interferon, an entry inhibitor, a small molecule immunomodulatory, and oligonucleotide therapy, wherein the oligonucleotide therapy is optionally selected from an additional antisense oligonucleotide (ASO), a short interfering nucleic acid (siNA), NAPs, or STOPS™. In some embodiments, the additional therapeutic agent is selected from the group consisting of ALG-000184, ALG-125755, recombinant interferon alpha 2b, IFN-α, PEG-IFN-α-2a, PEG-INF-2b, Pegbing (Mipeginterferon alfa-2b), lamivudine, telbivudine, adefovir dipivoxil, clevudine, entecavir, tenofovir alafenamide, tenofovir disoproxil, JNJ-3989 (ARO-HBV, or GSK5637608), GSK3228836, REP-2139, REP-2165, VIR-2218 (BRII-835, or Elebsiran), AB-729 (Imdurisan), DCR-HBVS (RG6346 or Xalnesiran), BW-20507 (Argo HBV siRNA), HT-101 (Hepa Thera HBV siRNA), OLX703A (Olix HBV siRNA), HRS-5635 (Hengrui HBV siRNA), RBD1016 (Ribo HBV siRNA), TQA3038 (ChiaTai Tianqing HBV siRNA), GLS4, NZ-4, RG7907, EDP-514, ABI-H03733, ABI-H2158, ZM-H1505R, ABI-4334 (CAMs), and ABI-6250 (HDV entry inhibitor). In some embodiments, the ASO and the additional therapeutic agent are administered concurrently or consecutively. In some embodiments, the treatment results in reducing a viral load of HBV in the subject, reducing a level of a virus antigen in the subject, or a combination thereof. In some embodiments, the treatment results in increased toll-like receptor 8 (TLR8) activity. In some embodiments, the subject is a mammal, optionally a human.

In another aspect, the present disclosure provides an antisense oligonucleotide (ASO), comprising:
(a) a 5′-wing region (A′) comprising 2 to 7 nucleotides;
(b) a central region (B′) comprising up to 16 positions comprising at least one abasic monomer and 5 to 15 nucleotides; and
(c) a 3′-wing region (C′) comprising 2 to 7 nucleotides.

In some embodiments, the 1 to 7 nucleotides of the 5′-wing region (A′) are locked nucleotides or substituted nucleotides. In some embodiments, the 1 to 7 nucleotides of the 3′-wing region (C′) are locked nucleotides or substituted nucleotides.

In some embodiments, the at least one abasic monomer has a structure of

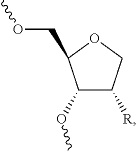

wherein R is H, alkyl (e.g., CH₃), an alkoxy (e.g., O—CH₃ or MOE), O-cyp, or O-mcyp or R can connect to the 4′ of the sugar to form a locked abasic monomer, and wherein [symbol] represents a phosphodiester linkage, a phosphorothioate linkage, a mesyl phosphoroamidate linkage, or H. In some embodiments, the at least one abasic monomer is selected from a 2′-deoxy abasic monomer or a 2′-substituted abasic monomer, such as abasic monomer 1, abasic monomer 2, abasic monomer 3, and abasic monomer 4.

In some embodiments, the central region (B′) comprises 10 positions (e.g., one abasic monomer and nine nucleotides) and the at least one abasic monomer is located at any one of positions 4, 5, or 6 of the central region (B′).

In some embodiments, the 5′-wing region (A′) and the 3′-wing region (C′) each independently comprise 5 nucleotides. In some embodiments, the 5 nucleotides comprise locked nucleotides, 2′-MOE nucleotides, or a combination thereof.

In some embodiments, the 5 to 15 nucleotides of the central region (B′) are DNA.

In some embodiments, at least one and up to all linkages in the ASO are phosphorothioate linkages.

In some embodiments, the central region (B′) contains only one abasic monomer. In some embodiments, the central region (B′) contains 2, 3, or 4 abasic monomers.

In some embodiments, (i) the 5′-wing region (A′) comprises one or two locked nucleic acids (LNAs); (ii) the 3′-wing region (C′) comprises one or two LNAs; or (iii) the 5′-wing region (A′) comprises one LNA and the 3′-wing region (C′) comprises one LNA.

In some embodiments, wherein the 5′-wing region (A′) and the 3′-wing region (C′) each independently comprise five 2′-MOE nucleotides, the central region (B′) comprises 10 positions and the at least one abasic monomer is located at any one of positions 4, 5, or 6 of the central region (B′), wherein all linkages in the ASO are phosphorothioate linkages; and, optionally, wherein: (i) the 5′-wing region (A′) comprises one or two locked nucleic acids (LNAs); (ii) the 3′-wing region (C′) comprises one or two LNAs; or (iii) the 5′-wing region (A′) comprises one LNA and the 3′-wing region (C′) comprises one LNA.

In some embodiments:
(a) the 5′-wing region (A′) and the 3′-wing region (C′) each independently comprise five 2′-MOE nucleotides, the central region (B′) comprises 10 positions consisting of one abasic monomer 1 at position 4 and the remaining positions being DNA nucleotides, wherein all linkages in the ASO are phosphorothioate linkages;
(b) the 5′-wing region (A′) and the 3′-wing region (C′) each independently comprise five 2′-MOE nucleotides, the central region (B′) comprises 10 positions consisting of one abasic monomer 1 at position 5 and the remaining positions being DNA nucleotides, wherein all linkages in the ASO are phosphorothioate linkages;
(c) the 5′-wing region (A′) and the 3′-wing region (C′) each independently comprise five 2′-MOE nucleotides, the central region (B′) comprises 10 positions consisting of one abasic monomer 1 at position 6 and the remaining positions being DNA nucleotides, wherein all linkages in the ASO are phosphorothioate linkages;
(d) the 5′-wing region (A′) and the 3′-wing region (C′) each independently comprise five 2′-MOE nucleotides, the central region (B′) comprises 10 positions consisting of one abasic monomer 2 at position 4 and the remaining positions being DNA nucleotides, wherein all linkages in the ASO are phosphorothioate linkages;
(e) the 5′-wing region (A′) and the 3′-wing region (C′) each independently comprise five 2′-MOE nucleotides, the central region (B′) comprises 10 positions consisting of one abasic monomer 2 at position 5 and the remaining positions being DNA nucleotides, wherein all linkages in the ASO are phosphorothioate linkages;
(f) the 5′-wing region (A′) and the 3′-wing region (C′) each independently comprise five 2′-MOE nucleotides, the central region (B′) comprises 10 positions consisting of one abasic monomer 2 at position 6 and the remaining positions being DNA nucleotides, wherein all linkages in the ASO are phosphorothioate linkages;
(g) the 5′-wing region (A′) comprises five 2′-MOE nucleotides; the 3′-wing region (C′) comprises five positions comprising 2′-MOE nucleotides at positions 1, 2, and 4 and LNAs at positions 3 and 5; and the central region (B′) comprises 10 positions consisting of one abasic monomer 1 at position 6 and the remaining positions being DNA nucleotides, wherein all linkages in the ASO are phosphorothioate linkages; or
(h) the 5′-wing region (A′) comprises five positions comprising 2′-MOE nucleotides at positions 1, 2, 4, and 5 and an LNAs at position 3; the 3′-wing region (C′) comprises five positions comprising 2′-MOE nucleotides at positions 1, 2, 4, and 5 and an LNA at position 3; the central region (B′) comprises 10 positions consisting of one abasic monomer 2 at position 6 and the remaining positions being DNA nucleotides, wherein all linkages in the ASO are phosphorothioate linkages.

In some embodiments, the ASO is selected from ASO-676 (SEQ ID NO: 697), ASO-677 (SEQ ID NO: 698), ASO-1037 (SEQ ID NO: 1058), ASO-707 (SEQ ID NO: 728), ASO-1192 (SEQ ID NO: 1213), ASO-651 (SEQ ID NO: 672), ASO-1166 (SEQ ID NO: 1187), ASO-1181 (SEQ ID NO: 1202), ASO-1179 (SEQ ID NO: 1200), and ASO-962 (SEQ ID NO: 983).

In some embodiments, the ASO further comprises a conjugate attached to the 5′ end or 3′ end of the ASO. In some embodiments, the conjugate comprises a GalNAc. In some embodiments, the GalNAc is a monomeric GalNAc. In some embodiments, the GalNAc is GalNAc 4 (i.e., the GalNAc of Formula VII, wherein n=1 and R^{z}=OH).

Disclosed herein is a use of an ASO according to any one of aspects or embodiments described above in the manufacture of a medicament for treating an HBV infection.

Disclosed herein is the ASO of any one of the aspects or embodiments described above for treating an HBV infection.

### BRIEF DESCRIPTION OF THE DRAWINGS

FIG. 1A-1E: FIGS. 1A, 1B, 1C, 1D, and 1E show the results of assays measuring maximum human toll-like receptor (TLR) activity in response to treatment with various disclosed ASOs at the dose range of 10 μM, 1 μM, 0.1 μM, 0.01 μM and 0 μM. Specifically, FIG. 1A shows maximum human TLR3 activity with poly AU as positive control, FIG. 1B shows maximum human TLR4 activity with LPS as positive control, FIG. 1C shows maximum human TLR7 activity with R848 as positive control, FIG. 1D shows maximum human TLR8 activity with small molecule GS-9688 and oligonucleotide poly U as positive controls, and FIG. 1E shows maximum human TLR9 activity with ODN2006 as positive control.

FIG. 2 shows the percentage body weight change of mice treated with PBS (Group 1), ASO-1 (Group 3), ASO-ASO-139 (Group 9) and ASO-6 (Group 5) after treatment at 50 mg/kg on day 0, day 3, day 7, day 10, day 14, and day 21 in AAV-HBV infected human TLR8 knock-in mice.

FIG. 3A-3C depicts the effects of a mono GalNAc modification on ASO-6. FIG. 3A shows an exemplary ASO-651 molecule produced from the parental strain ASO-6. Figure discloses SEQ ID NOS 7 and 672, respectively. FIG. 3B shows the results of the amount of both ASO-6 and ASO-651 uptake in macrophages after 1 hour or 24 hours treatment of 1 uM or 10 uM. FIG. 3C shows the effect of 5 nM, 14 nM, 41 nM, 123 nM, 370 nM, 1111 nM, 3333 nM, and 10000 nM of ASO-6 and ASO-651 in HEK-Blue hTLR8 cells on hTLR8 agonist activity which was monitored through SEAP reporter assay.

FIG. 4A-4C unconjugated ASO (ASO-6) vs Mono GalNAc ASO (ASO-651) PK/PD effects in hTLR8 Knock-in mice. FIG. 4A shows the effect of ASO-6 and ASO-651 on the kidney/liver ratio on uninfected mice 4 hours post treatment at 40 mg/kg and FIG. 4B shows the effect of ASO-6 and ASK-651 on the kidney/liver ratio on AAV-HBV infected mice 4 hours post treatment at 40 mg/kg. FIG. 4C shows the PD effect of ASO-6 and ASO-651 on the uninfected hTLR8 knock-in mice IL-12 p40 profile 4 hours post 40 mg/kg treatment (FIG. 4C).

FIG. 5 shows the effects of ASO-182 and ASO-222, as well as ASO-1 positive control and PBS negative control, on HBsAg (left graph) and Terminal Human ALT1 (right graph) in PXB mice with humanized livers injected with 7×50 mg/kg per dose of PBS or ASO over a time course of 28 days.

FIG. 6 shows the effect of ASO-1, ASO-139, ASO-6, ASO-114, ASO-153, ASO-182, ASO-222 4 hours post a single dose of 40 mg/kg in AAV-HBV infected hTLR8 knock in mice on liver protein levels of IL-12 p40 (top left), IP-10 (top right), IL-18 (bottom left) and TNF-alpha (bottom right) measured through Luminex mouse multiplex panel.

FIG. 7 shows exemplary ASO molecules produced from the parental strain ASO-6 using two LNA modifications selected from a pool of ASOs designed through LNA walk. Figure discloses SEQ ID NOS 7, 183, 576, 580, and 577, respectively, in order of appearance.

FIG. 8 depicts the experimental design of evaluating HBSAG, HBeAg, HBV DNA, and ALT levels in the AAV-HBV infected C57BL/6 mouse model.

FIG. 9 shows the effect of ASO-555, ASO-556, and ASO-559 on HBsAg levels in plasma (left) and HBeAg levels in plasma (right) in an AAV-HBV C57BL/6 mouse model. Mice were injected with 40 mg/kg on days 0, 3, 7, 10, 14, and 21. On day 7, 14, and 21, plasma was collected and tested for HBsAg and HBeAg. Group 1 was injected with PBS, Group 2 with ASO-1 control, Group 3 with ASO-139 control, Group 4 with ASO-555, Group 5 with ASO-556, and Group 6 with ASO-559.

FIG. 10 shows the effects of ASO-6, ASO-555, and ASO-556 on liver cytokine proteins in an uninfected hTLR8 knock in mouse model. Mice were treated with a single dose of 40 mg/kg of either PBS, ASO-1, ASO-6, ASO-555, and ASO-556. Levels of IFN alpha (top left), IFN beta (top right), IL-12/IL23 p40 (bottom left), and TNF alpha (bottom right) protein levels were determined at 4 hours post dose. *p<0.05 **p<0.005.

FIG. 11 shows the effects of ASO-713 and ASO-714 on HBsAg levels in plasma in a mouse model. Mice were injected with 25 mg/kg of PBS (Group 1), ASO-1 control (Group 2), ASO-139 control (Group 4), ASO-713 (Group 11), and ASO-714 (Group 12), on days 0, 3, 7, 10, 14, and 21, and HBsAg IU/ml in mouse plasma at various time points was measured.

FIG. 12 shows exemplary ASO molecules produced from the parental strain ASO-1 with 2′-OMe Abasic modifications in the gap region. Figure discloses SEQ ID NOS 693-702, respectively, in order of appearance.

FIG. 13 shows the effects of ASO-672 and ASO-677 on HBsAg levels in plasma (left) and HBeAg levels in plasma (right), as well as liver and kidney ASO concentrations (bottom) in an AAV-HBV mouse model. Mice were injected with PBS as a negative control (Group 1), and 6×40 mg/kg per dose of ASO-1 control (Group 2), ASO-139 control (Group 3), ASO-672 (Group 7), and ASO-677 (Group 8), on days 0, 3, 7, 10, 14, and 21. HBsAg IU/ml and HBeAg PEIU/mL at various time points are presented. Liver and kidney ASO concentrations were determined through LCMS at the conclusion of the 28 day study.

FIG. 14 shows the effects of ASO-673, ASO-674, ASO-675, ASO-676, ASO-678, ASO-679, ASO-683, ASO-684, ASO-686, ASO-687, ASO-690, ASO-691, ASO-692 and ASO-693 on HBsAg levels in plasma, as well as liver and kidney ASO concentrations for ASO-676 in a n AAV-HBV mouse model. For the top right panel, mice were injected with of PBS (Group 1) or 6×25 mg/kg of ASO-1 control, ASO-673, ASO-674, ASO-675, ASO-676, ASO-678, and ASO-679, and HBsAg levels were assessed on days 0, 3, 7, 10, 14, 21, and 28. In the bottom right panel, HBsAg was measured at various time points. Liver and kidney ASO concentrations were determined at the conclusion of the 28 day study. The left-side panels show the effects of ASO-683, ASO-684, ASO-686, ASO-687, ASO-690, ASO-691, ASO-692 and ASO-693 on levels of HBsAg in AAV-HBV mouse model. AAV-HBV mice were injected subcutaneously with PBS (negative control) or 6×25 mg/kg ASOs including ASO-1 and ASO-139 as positive controls on days 0, 3, 7, 10, 14 and 21. Plasma from days 0, 7, 14, 21 and 28 were used for HBsAg ELISA measurement

FIG. 15 shows exemplary ASO molecules produced from the parental construct ASO-1 with 2′-deoxy abasic monomer modifications in the gap regions. Figure discloses SEQ ID NOS 2, and 723-732, respectively, in order of appearance.

FIG. 16 shows the effects of ASO-704, ASO-706, ASO-707, and ASO-710 on HBsAg levels in plasma as well as liver and kidney ASO concentrations in an AAV-HBV mouse model. Mice were injected with PBS (Group 1) or 6×25 mg/kg of ASOs including ASO-1 control (Group 2), ASO-139 control (Group 4), ASO-704 (Group 13), ASO-706 (Group 14), ASO-707 (Group 15), and ASO-710 (Group 16) on days 0, 3, 7, 10, 14, and 21, and HBsAg was measured weekly at various time points. Liver and kidney ASO concentrations were determined at the conclusion of the 28 day study.

FIG. 17 shows exemplary ASO molecules produced from the parental construct ASO-672 with 2′-OMe abasic monomer modifications in the gap regions and LNA modifications in various positions. Figure discloses SEQ ID NOS 693 and 807-818, respectively, in order of appearance.

FIG. 18 shows exemplary ASO molecules produced from the parental strain ASO-672 with 2′-OMe abasic monomer modifications in the gap regions and LNA modifications in various positions. Figure discloses SEQ ID NOS 819-831, respectively, in order of appearance.

FIG. 19 shows exemplary ASO molecules produced from the parental strain ASO-672 with 2′-OMe abasic monomer modifications in the gap regions and LNA modifications in various positions. Figure discloses SEQ ID NOS 832-841, respectively, in order of appearance.

FIG. 20 shows exemplary ASO molecules produced from the parental strain ASO-672 with 2′-OMe abasic monomer modifications in the gap regions and LNA modifications in various positions. Figure discloses SEQ ID NOS 842-851, respectively, in order of appearance.

FIG. 21 shows exemplary ASO molecules produced from the parental strain ASO-677 with 2′-OMe abasic monomer modifications in the gap regions and additional LNA modifications in various positions. Figure discloses SEQ ID NOS 698, and 981-986, respectively, in order of appearance.

FIG. 22 shows exemplary ASO molecules with a 5′-mono-GalNac modification produced from the parental construct ASO-962, ASO-963, or ASO-965 that have with 2′-OMe abasic monomer modifications in the gap regions and LNA modifications. Figure discloses SEQ ID NOS 983-986, 1088, and 1154-1155, respectively, in order of appearance.

FIG. 23 shows the effects of ASO-962, ASO-1067, ASO-963, ASO-1133, ASO-965, and ASO-1134 on HBsAg levels in plasma in AAV-HBV C57BL/6 mice. Mice were treated with either PBS (Group 1), ASO-1 (Group 2; 6×25 mg/kg), ASO-1 (Group 3; 6×40 mg/kg), ASO-139 (Group 4; 6×25 mg/kg), ASO-962 (Group 5; 6×25 mg/kg), ASO-1067 (Group 6; 6×25 mg/kg), ASO-963 (Group 7; 6×25 mg/kg), ASO-1133 (Group 8; 6×25 mg/kg), ASO-965 (Group 9; 6×25 mg/kg), and ASO-1134 (Group 10; 6×25 mg/kg). HBsAg in mouse plasma was measured on day 7 post Day 0 and D3 two treatments.

FIG. 24 shows the effects of ASO-962 and ASO-1067 on HBsAg levels in plasma in AAV-HBV mouse model. Mice were injected with PBS (Group 1), or 6×25 mg/kg ASOs as follows: ASO-1 control (Group 2), ASO-139 control (Group 4), ASO-962 (Group 5), and ASO-1067 (Group 6). Subcutaneous dosing occurred on days 0, 3, 7, 10, 14, and 21, and HBsAg was measured weekly at various time points.

FIG. 25 shows the effects of ASO-963 and ASO-1133 on HBsAg levels in plasma in AAV-HBV mouse model. Mice were injected with PBS (Group 1), or 6×25 mg/kg ASOs as follows: ASO-1 control (Group 2), ASO-139 control (Group 4), ASO-963 (Group 7), and ASO-1133 (Group 8). Subcutaneous dosing occurred on days 0, 3, 7, 10, 14, and 21, and HBsAg was measured weekly at various time points.

FIG. 26 shows the effects of ASO-965 and ASO-1134 on HBsAg levels in plasma in AAV-HBV mouse model. Mice were injected with PBS (Group 1) or 6×25 mg/kg ASOs as follows: ASO-1 control (Group 2), ASO-139 control (Group 4), ASO-965 (Group 9), and ASO-1134 (Group 10). Subcutaneous dosing occurred on days 0, 3, 7, 10, 14, and 21, and HBsAg was measured weekly at various time points.

FIG. 27 shows exemplary ASO molecules with a LNA modification patterns from the parental constructs ASO-706 and ASO-707. Figure discloses SEQ ID NOS 727, 1005-1010, 728, and 1188-1193, respectively, in order of appearance.

FIG. 28 shows exemplary ASO molecules with a 2×LNA modification patterns from the parental strain ASO-677. Figure discloses SEQ ID NOS 1021-1028, respectively, in order of appearance.

FIG. 29 shows exemplary ASO molecules with a 2×LNA modification patterns from the parental strain ASO-677. Figure discloses SEQ ID NOS 1029-1036, respectively, in order of appearance.

FIG. 30 shows exemplary ASO molecules with a 2×LNA modification patterns from the parental strain ASO-677. Figure discloses SEQ ID NOS 1037-1044, respectively, in order of appearance.

FIG. 31 shows exemplary ASO molecules with a 2×LNA modification patterns from the parental strain ASO-677. Figure discloses SEQ ID NOS 1045-1052, respectively, in order of appearance.

FIG. 32 shows exemplary ASO molecules with a 2×LNA modification patterns from the parental strain ASO-677. Figure discloses SEQ ID NOS 1053-1059, respectively, in order of appearance.

FIG. 33 shows exemplary ASO molecules with a 1×LNA modification patterns from the parental strain ASO-677. Figure discloses SEQ ID NOS 698, and 1060-1067, respectively, in order of appearance.

FIG. 34 shows exemplary ASO molecules based on ASO-1 designed using a 3′ abasic walk. Figure discloses SEQ ID NOS 2, and 784-788, respectively, in order of appearance.

FIG. 35 shows the results of % inhibition in RNaseH activity after treatment of the ASOs prepared using a 3′ wing abasic walk based on ASO-1 at 0.1, 0.2, 1, 2, 3, 4, 5, 10, 20, 30, 100, and 200 nM.

FIG. 36 shows the results of % viability after treatment of the ASOs prepared using a 3′ wing abasic walk based on ASO-1 at 0.1, 0.2, 1, 2, 3, 4, 5, 10, 20, 30, 100, and 200 nM.

FIG. 37 shows exemplary ASO molecules produced from the parental strain ASO-1 with 2′-OMe (abasic monomer 1) or 2′deoxy abasic (abasic monomer 2) monomer modifications in the gap regions and additional LNA modifications in various positions. Figure discloses SEQ ID NOS 2, 140, 697-698, 1057-1058, 728, 1213-1214, and 1212, respectively, in order of appearance.

FIG. 38 shows the effects of ASO-1, ASO-139, and ASO-677 on HBsAg levels in plasma in AAV-HBV mouse model. Mice were injected with PBS (Group 1), and 6×40 mg/kg ASOs including ASO-1 control (Group 2), ASO-139 control (Group 3), and ASO-677 (Group 4) on days 0, 3, 7, 10, 14, and 21, and HBsAg was measured weekly at various time points.

FIG. 39 shows the effects of ASO-1, ASO-139, and ASO-677 on Alanine Aminotransferase (ALT) activity in plasma in a mouse model. Mice were injected with PBS (Group 1), and 6×40 mg/kg ASOs including ASO-1 control (Group 2), ASO-139 control (Group 3), and ASO-677 (Group 4) on days 0, 3, 7, 10, 14, and 21, and ALT was measured weekly at various time points.

FIG. 40 shows the tissue concentrations of ASO-1, ASO-139, and ASO-677 in the liver and the kidney of a mouse model. Mice were injected with 40 mg/kg of ASO-1 control, ASO-139 control, or ASO-677 on days 0, 3, 7, 10, 14, and 21, and tissues were collected on Day 28 and ASO concentrations in livers and kidneys were measured by LCMS.

FIG. 41 shows the effects of ASO-1, ASO-139, and ASO-1036 on HBsAg levels in plasma in AAV-HBV mouse model. Mice were injected with PBS (Group 1), and 6×25 mg/kg ASOs including ASO-1 control (Group 2), ASO-139 control (Group 3), and ASO-1036 (Group 4) on days 0, 3, 7, 10, 14, and 21, and HBsAg was measured weekly at various time points.

FIG. 42 shows the effects of ASO-1, ASO-139, and ASO-1036 on Alanine Aminotransferase (ALT) activity in plasma in AAV-HBV mouse model. Mice were injected with PBS (Group 1), and 6×25 mg/kg ASOs including ASO-1 control (Group 2), ASO-139 control (Group 3), and ASO-1036 (Group 4) on days 0, 3, 7, 10, 14, and 21, and ALT was measured weekly at various time points.

FIG. 43 shows the effects of ASO-1, ASO-139, and ASO-1037 on HBsAg levels in plasma in AAV-HBV mouse model. Mice were injected with PBS (Group 1), and 6×25 mg/kg ASOs including ASO-1 control (Group 2), ASO-139 control (Group 3), and ASO-1037 (Group 4) on days 0, 3, 7, 10, 14, and 21, and HBsAg was measured weekly at various time points.

FIG. 44 shows the effects of ASO-1, ASO-139, and ASO-1037 on Alanine Aminotransferase (ALT) activity in plasma in AAV-HBV mouse model. Mice were injected with PBS (Group 1), and 6×25 mg/kg ASOs including ASO-1 control (Group 2), ASO-139 control (Group 3), and ASO-1037 (Group 4) on days 0, 3, 7, 10, 14, and 21, and ALT was measured weekly at various time points.

FIG. 45 shows the effects of ASO-1, ASO-139, and ASO-707 on HBsAg levels in plasma in AAV-HBV mouse model. Mice were injected with PBS (Group 1), and 6×25 mg/kg ASOs including ASO-1 control (Group 2), ASO-139 control (Group 3), and ASO-707 (Group 4) on days 0, 3, 7, 10, 14, and 21, and HBsAg was measured weekly at various time points.

FIG. 46 shows the effects of ASO-1, ASO-139, and ASO-707 on Alanine Aminotransferase (ALT) activity in plasma in AAV-HBV mouse model. Mice were injected with PBS (Group 1), and 6×25 mg/kg ASOs including ASO-1 control (Group 2), ASO-139 control (Group 3), and ASO-707 (Group 4) on days 0, 3, 7, 10, 14, and 21, and ALT was measured weekly at various time points.

FIG. 47 shows the tissue concentrations of ASO-1, ASO-139, and ASO-707 in the livers and the kidneys of AAV-HBV mouse model. Mice were injected with 6×25 mg/kg of ASO-1 control, ASO-139 control, or ASO-707 on days 0, 3, 7, 10, 14, and 21, and tissues were collected on day 28 and ASO concentrations were measured using LCMS.

FIG. 48 shows the effects of ASO-139 and ASO-1192 on HBsAg levels in plasma in AAV-HBV mouse model. Mice were injected with PBS (Group 1), and 2×25 mg/kg ASOs including ASO-139 control (Group 2), and ASO-1192 (Group 3) on days 0, 3, and HBsAg was measured weekly at various time points.

FIG. 49 shows the effects ASO-139 and ASO-1192 on Alanine Aminotransferase (ALT) activity in plasma in AAV-HBV mouse model. Mice were injected with PBS (Group 1), and 2×25 mg/kg ASOs including ASO-139 control (Group 2), and ASO-1192 (Group 3) on days 0, 3, and ALT was measured weekly at various time points.

FIG. 50 shows the effects of ASO-1, ASO-139, and ASO-676 on HBsAg levels in plasma in AAV-HBV mouse model. Mice were injected with f PBS (Group 1), and 6×25 mg/kg ASOs including ASO-1 control (Group 2), ASO-139 control (Group 3), and ASO-676 (Group 4) on days 0, 3, 7, 10, 14, and 21, and HBsAg was measured weekly at various time points.

FIG. 51 shows the effects of ASO-1, ASO-139, and ASO-676 on Alanine Aminotransferase (ALT) activity in plasma in AAV-HBV mouse model. Mice were injected with PBS (Group 1), and 6×25 mg/kg ASOs including ASO-1 control (Group 2), ASO-139 control (Group 3), and ASO-676 (Group 4) on days 0, 3, 7, 10, 14, and 21, and ALT was measured weekly at various time points.

FIG. 52A-52D show the effects of ASO-1 and ASO-676 on IL-12/IL-23p40 and IP-10 levels in the plasma and liver of hTLR8 knock in mouse model. FIG. 52A shows concentration of IL-12/IL-23p40 in mouse plasma after treatment with PBS, ASO-1, or ASO-676. FIG. 52B shows concentration of IP-10 (CXCL10) in mouse plasma after treatment with PBS, ASO-1, or ASO-676. FIG. 52C shows concentration of IL-12/IL-23p40 in mouse liver after treatment with PBS, ASO-1, or ASO-676. FIG. 52D shows concentration of IP-10 (CXCL10) in mouse liver after treatment with PBS, ASO-1, or ASO-676. For each assessment, mice were injected with PBS (Group 1), and single dose 40 mg/kg ASOs including ASO-1 control (Group 2), or ASO-676 (Group 3) on days 0, and IL-12/IL23p40 or IP-10 (CXCL10) concentrations were measured 4 hours following the treatment.

FIG. 53 shows the effects of ASO-139 and ASO-1191 on HBsAg levels in plasma in AAV-HBV mouse model. Mice were injected with PBS (Group 1), and 2×25 mg/kg ASOs including ASO-139 control (Group 2), and ASO-1191 (Group 3) on days 0 and 3 and HBsAg was measured weekly at various time points.

FIG. 54 shows the effects of ASO-139 and ASO-1191 on Alanine Aminotransferase (ALT) activity in plasma in AAV-HBV mouse model. Mice were injected with PBS (Group 1), and 2×25 mg/kg ASOs including ASO-139 control (Group 2), and ASO-1191 (Group 3) on days 0 and 3 and ALT was measured weekly at various time points.

FIG. 55A-55D show the effects of ASO-1 and ASO-1191 on IL-12/IL-23p40 and IP-10 levels in the plasma and liver of hTLR8 knock in mouse model. FIG. 55A shows concentration of IL-12/IL-23p40 in mouse plasma after treatment with PBS, ASO-1, or ASO-1191. FIG. 55B shows concentration of IP-10 (CXCL10) in mouse plasma after treatment with PBS, ASO-1, or ASO-1191. FIG. 55C shows concentration of IL-12/IL-23p40 in mouse liver after treatment with PBS, ASO-1, or ASO-1191. FIG. 55D shows concentration of IP-10 (CXCL10) in mouse liver after treatment with PBS, ASO-1, or ASO-1191. For each assessment, mice were injected with PBS (Group 1) and single dose of 40 mg/kg ASOs including, ASO-1 control (Group 2), or ASO-676 (Group 3) on days 0, and IL-12/IL23p40 or IP-10 (CXCL10) concentrations in plasma and liver were measured 4 hours following the treatment.

FIG. 56 shows the effects of ASO-139 and ASO-1193 on HBsAg levels in plasma in AAV-HBV mouse model. Mice were injected with PBS (Group 1), and 2×25 mg/kg ASOs including ASO-139 control (Group 2), and ASO-1193 (Group 3) on days 0 and 3 and HBsAg was measured weekly at various time points.

FIG. 57 shows the effects of ASO-139 and ASO-1193 on Alanine Aminotransferase (ALT) activity in plasma in AAV-HBV mouse model. Mice were injected with PBS (Group 1), and 2×25 mg/kg ASOs including ASO-139 control (Group 2), and ASO-1193 (Group 3) on days 0 and 3 and ALT was measured weekly at various time points.

FIG. 58A-58D show the effects of ASO-1 and ASO-1193 on IL-12/IL-23p40 and IP-10 levels in the plasma and liver of hTLR8 knock in mouse model. FIG. 58A shows concentration of IL-12/IL-23p40 in mouse plasma after treatment with PBS, ASO-1, or ASO-1193. FIG. 58B shows concentration of IP-10 (CXCL10) in mouse plasma after treatment with PBS, ASO-1, or ASO-1193. FIG. 58C shows concentration of IL-12/IL-23p40 in mouse liver after treatment with PBS, ASO-1, or ASO-1193. FIG. 58D shows concentration of IP-10 (CXCL10) in mouse liver after treatment with PBS, ASO-1, or ASO-1193. For each assessment, mice were injected with PBS (Group 1), and single dose 40 mg/kg ASOs including ASO-1 control (Group 2), or ASO-676 (Group 3) on days 0, and IL-12/IL23p40 or IP-10 (CXCL10) concentrations were measured 4 hours following the treatment.

FIG. 59 shows exemplary ASO molecules with LNA modification patterns based on the parental construct ASO-707. Figure discloses SEQ ID NOS 1217, 1212, 1218, 1213-1214, and 1220, respectively, in order of appearance.

FIG. 60 shows exemplary ASO molecules with LNA modification patterns (including a single LNA) based on the parental construct ASO-707. Figure discloses SEQ ID NOS 1221-1230, respectively, in order of appearance.

FIG. 61 shows exemplary ASO molecules with LNA modification patterns (including two LNAs) based on the parental construct ASO-707. Figure discloses SEQ ID NOS 1231-1240, respectively, in order of appearance.

FIG. 62 shows exemplary ASO molecules with LNA modification patterns (including two LNAs) based on the parental construct ASO-707. Figure discloses SEQ ID NOS 1241-1250, respectively, in order of appearance.

FIG. 63A-63D show the effects of ASO-1 and ASO-677 on IL-12/IL-23p40, TNF alpha and IP-10 levels in the plasma and liver of hTLR8 knock in mouse model. FIG. 63A shows concentration of IL-12/IL-23p40 in mouse plasma after treatment with PBS, ASO-1, or ASO-677. FIG. 63B shows concentration of IL-12/IL-23p40 in mouse liver after treatment with PBS, ASO-1, or ASO-677. FIG. 63C shows concentration of IP-10 (CXCL10) in mouse liver after treatment with PBS, ASO-1, or ASO-677. FIG. 63D shows concentration of TNF alpha in mouse liver after treatment with PBS, ASO-1, or ASO-677. For each assessment, mice were injected with PBS (Group 1), and single dose 40 mg/kg ASOs including ASO-1 control (Group 2), or ASO-677 (Group 3) on days 0, and IL-12/IL23p40 or IP-10 (CXCL10) or TNF alpha concentrations were measured 4 hours following the treatment.

FIG. 64 shows 5′mono GalNAc ASO Day 7 AAV-HBV Mouse Study Results. The provided results show HBsAg levels, which were significantly lower in those ASOs containing a mono GalNAc relative to the reference ASOs (ASO-1 and ASO-139).

FIG. 65 shows unconjugated ASO Day 7 AAV-HBV Mouse Study Results. The provided results show HBsAg levels, which were modestly better for some of the tested ASOs relative to the reference ASOs (ASO-1 and ASO-139).

FIG. 66 shows ASO Day 7 AAV-HBV Mouse Study Results. The provided results show HBsAg levels. Several of the tested unconjugated ASOs, including ASO-1191, ASO-1197, ASO-1192, and ASO-1193, outperformed the reference ASOs (ASO-1 and ASO-139). Two 5′ Mono GalNAc conjugated ASOs ASO-1179 and ASO-1181 are among the most potent ASOs, outperforming ASO-1 and ASO-139.

FIG. 67 shows the results of an RNaseH biochemistry assay. The electrophoresis gel patterns clearly differentiated ASOs with abasic modifications from two control ASOs without abasic modification, thus indicating that abasic modifications altered the enzyme cleavage pattern.

FIG. 68 shows in vitro and in vivo activity data obtained for ASO-1196 (SEQ ID NO: 1217).

FIG. 69 shows in vitro and in vivo activity data obtained for ASO-1197 (SEQ ID NO: 1218).

FIG. 70 shows result from a PD study in mice with a hTLR8 knock-in gene. ASOs with monomeric or dimeric GalNAc (specifically, GalNAc 4) were administered and Interferon-α and Interferon-β levels were assessed. The result indicate the monomeric GalNAc strikes a good balance between RNaseH (shown in other figures) and immune response. Figure discloses SEQ ID NOS 7, and 672-677, respectively, in order of appearance.

### DETAILED DESCRIPTION

The present disclosure is directed to modified antisense oligonucleotides (ASOs) and pharmaceutical compositions comprising the same. The present disclosure is also directed to methods and uses of the antisense oligonucleotides and pharmaceutical compositions for treating or preventing hepatitis B virus (HBV) infection in particular.

The disclosed ASOs can contain 14-23 nucleotide units, and the ASOs can contain: (a) a central region comprising 6 or more contiguous DNA nucleosides, (b) a 5′-wing region comprising 2 to 7 locked nucleosides or 2′ substituted nucleosides, and (c) a 3′-wing region comprising 2 to 7 locked nucleosides or 2′ substituted nucleosides.

Without being bound by this theory, the mechanisms of action for the ASO are thought to be twofold: 1) ASO hybridizes to target RNA through Watson-Crick base pairing. The DNA-RNA heteroduplex would recruit RNase H in the cell which subsequently cleaves the RNA in the heteroduplex; 2) ASO with certain sequence motifs and chemical modifications are recognized by Pattern Recognition Receptors (PRRs) of innate immune system. PRRs are proteins capable of recognizing molecules frequently found in pathogens (the so-called Pathogen-Associated Molecular Patterns-PAMPs), or molecules released by damaged cells (the Damage-Associated Molecular Patterns-DAMPs). Toll-like receptors (TLRs) are a family of 13 type 1 transmembrane proteins that belong to PRRs. Nucleic acids (NAs) are sensed by a subfamily of TLRs including TLR3, TLR7, TLR8, TLR9, and TLR13. These NA-sensing TLRs are localized in the endosomal compartment to prevent hazardous autoimmune responses.

Human TLR8 (hTLR8) is an endosomal receptor primarily expressed in monocytes/macrophages and myeloid dendritic cells. It recognizes viral and bacterial RNA and triggers production of various antiviral and immunomodulatory cytokines, including IL-12, IL-18, TNF-α, and IFN-γ. TLR8 agonists directly activate and promote the maturation of professional antigen-presenting cells (e.g. myeloid dendritic cells), and stimulate antigen-specific T-cell responses, including a CD8+ T-cell response to infected hepatocytes in chronic hepatitis B patients.

Previous studies have shown that HBV ASO GSK-836 with no GalNAc conjugation exhibited better clinical outcome than GSK-404 (the same ASO sequence and chemical modifications as GSK-836 but contains a GalNAc targeting group for hepatocyte delivery). In the Ph2b B-Clear Trial, 300 mg/week with loading doses for 24 weeks achieved 30% HBsAg<LLOQ at the end of dosing; 10% patients remained HBsAg<LLOQ after 24 weeks follow up. It was suggested that GSK-836 has human TLR8 agonist activity in addition to RNase H activity. Although the percent of patients who reached undetectable HBsAg is promising, there is likely room for improvement. Another area for improvement is the high percentage of non-responders in the treatment.

However, treatment of HBV with antisense oligonucleotides still exhibits some safety problems, including liver toxicity. In the GSK-836 Ph2b B-Clear trial, 9% of the patients dropped out of treatment due to drug adverse events. There is room for improvement in the GSK-836 safety profile.

Thus, there is a need in the art to discover antisense oligonucleotides that have improved safety profiles, and increased efficacy. In this application, at least one, and up to three aspects of the HBV ASO profile (RNase H activity, hTLR8 activity and liver safety) have been improved over GSK-836 through discovery of novel sequences and application of unique chemistries. Discovery of sequences and chemical modifications that improved one or more aspects of RNase H activity, hTLR8 activity and liver safety of ASO were unconventional and unexpected.

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein for the purpose of describing particular embodiments only and is not intended to be limiting.

### I. Definitions

Unless defined otherwise, all technical and scientific terms used herein generally have the same meaning as commonly understood by one of ordinary skill in the art to which this technology belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al., (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

Reference to “about” a value or parameter herein includes (and describes) variations that are directed to a recited value or parameter as well as the recited p value or parameter per se. The variations for the term “about” mean plus or minus ten percent (10%) of a value. Thus, for example, “about 100” refers to 100, as well as any number between 90 and 110.

It is understood that aspects and variations of the embodiments described herein include “consisting” and/or “consisting essentially of” aspects and variations. Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

As used herein, the terms “abasic monomer,” “abasic site,” “abasic position,” and “abasic residue” may be used interchangeably and refer to a sugar-phosphate backbone (e.g., a modified sugar-phosphate backbone) that lacks a base (either a purine or pyrimidine or non-natural base). For the purposes of the present disclosure, an “abasic monomer,” “abasic site,” “abasic position,” and “abasic residue” can comprise various chemical groups (e.g., alkyl, cycloalkyl, alkoxy, hydrogen, etc.) at the 2′ position of the sugar. The modification at the 2′ position may include an alkyl or alkoxy that is attached at both the 2′ and 4′ position of the sugar (e.g., a 2′-O, 4′-C-methylene linker). For the purposes of the present disclosure, these terms are also intended to include any stereoisomers of the sugar-phosphate backbone lacking a base. Specific examples of abasic monomers include abasic monomer 1, abasic monomer 2, abasic monomer 3, and abasic monomer 4, which are disclosed herein.

The terms “individual,” “subject,” and “patient” are used interchangeably herein and refer to any individual mammal, e.g., bovine, canine, feline, equine, simian, porcine, camelid, bat, or human, being treated according to the disclosed methods or uses. In preferred embodiments, the subject is a human.

As used herein, the term “effective amount” refers to the amount of a compound (e.g., an ASO of the present disclosure) sufficient to effect beneficial or desired results. An effective amount can be administered in one or more administrations, applications, or dosages and is not intended to be limited to a particular formulation or administration route.

As used herein, the term “treating” includes any effect (e.g., lessening, reducing, modulating, ameliorating, or eliminating) that results in the improvement of the condition, disease, disorder, and the like, or ameliorating a symptom thereof.

As used herein, the terms “alleviate” and “alleviating” refer to reducing the severity of the condition, such as reducing the severity by, for example, at least 10%, 20%, 30%, 40%, 50%, 60%, 70%, 80%, 90%, or 95%.

As used herein, the term “pharmaceutical composition” refers to the combination of an active agent (e.g., an ASO disclosed herein) with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, for example, Martin, Remington's Pharmaceutical Sciences, 15th Ed., Mack Publ. Co., Easton, PA [1975].

As used herein, the term “nucleobase” refers to a nitrogen-containing biological compound that forms a nucleoside. Examples of nucleobases include, but are not limited to, thymine, uracil, adenine, cytosine, guanine, and an analogue or derivative thereof.

For the purposes of the present disclosure, a DNA sequence that replaces all the U residues of an RNA sequence with T residues is “identical” to the RNA sequence, and vice versa. Accordingly, a sequence that is “identical to an RNA corresponding to” a DNA sequence constitutes the DNA sequence with all T replaced by U. The presence of modified nucleotides or 2′-deoxynucleotides in a sequence does not make a sequence not “identical to an RNA” but rather a modified RNA.

As used herein, “modified nucleotide” includes any nucleic acid or nucleic acid analogue residue that contains a modification or substitution in the chemical structure of an unmodified nucleotide base, sugar (including, but not limited to, 2′-substitution), or phosphate (including, but not limited to, alternate internucleotide linkers, such as phosphorothioates or the substitution of bridging oxygens in phosphate linkers with bridging sulfurs), or a combination thereof. Non-limiting examples of modified nucleotides are shown herein.

A target gene may be any gene in a cell or virus. Here, “target gene” and “target sequence” are used synonymously.

As used herein, a “conjugate” or “ligand” refers to any compound of molecule that is capable of interacting with another compound or molecule, directly or indirectly. The ligand may modify one or more properties of the ASO molecule to which it is attached, such as the pharmacodynamic, pharmacokinetic, binding, absorption, cellular distribution, cellular uptake, charge, and/or clearance properties of the ASO molecule. Non-limiting examples of such conjugates are described, e.g., in WO 2020/243490; WO 2020/097342; WO 2021/119325; PCT/US2021/019629; PCT/US2021/019628; PCT/US2021/021199; Sig. Transduct. Target Ther. 5 (101), 2020; ACS Chem. Biol. 10 (5), 1181-1187, 2015; J. Am. Chem. Soc. 136 (49), 16958-16961, 2014; Nucleic Acids Res. 42 (13), 8796-8807, 2014; Molec. Ther. 28 (8), 1759-1771, 2020; and Nucleic Acid Ther. 28 (3), 109-118, 2018, each of which is incorporated by reference herein.

As a general matter, compositions specifying a percentage are by weight unless otherwise specified. Further, if a variable is not accompanied by a definition, then the previous definition of the variable controls.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided may be different from the actual publication dates that may need to be independently confirmed.

### II. Antisense Oligonucleotides (ASOs)

Compounds of the present disclosure include modified antisense oligonucleotides (ASOs). The ASO can comprise 18 to 23 nucleotide units, e.g., 18, 19, 20, 21, 22 or 23 nucleotide units. The ASO can be a gapmer that comprises three regions: a 5′-wing region (A′), which optionally comprises modified nucleotides; a central gap region (B′), which optionally comprises nucleotides of a different type from the wings, e.g., nucleotides capable of inducing RNase H cleavage; and a 3′-wing region (C′), which optionally comprises modified nucleotides. Generally, the 5′-wing region (A′) and the 3′-wing region (C′) will comprise modified nucleotides.

The disclosed ASOs can comprise at least one modified nucleotide such as 2′ substituted nucleosides, including, but not limited to, 2′-MOE, 2′-O-cyp, 2′-O-mcyp, and 2′-OMe; 3′-modified nucleosides, including, but not limited to 3′-xylo; 2′ and 3′ substituted nucleosides, including, but not limited to, 2′-OMe-3′-xylo; locked nucleosides, including, but not limited to, a locked nucleic acid, including, but not limited to, LNA, ScpBNA, AmNA, and GuNA; modified nucleobases, including, but not limited to, (8nh)G, (8nh)A, (2s)T, and (5oh)C; modified linkages; or a combination thereof. The disclosed ASOs can comprise at least one phosphorothioate linkage or stereo-defined phosphorothioate linkage. The disclosed ASOs can comprise a combination of at least one modified nucleotide and at least one phosphorothioate linkage or stereo-defined phosphorothioate linkage. The structures of the modified nucleotides and the stereo-defined phosphorothioate linkages are described further below. Preparation of the modified nucleotide monomers, including, but not limited to, 2′-O-cyp, 2′-O-mcyp, 2′-OMe, and 2′-OMe-3′-xylo monomers, is disclosed in PCT/US2023/078224 (WO 2024/097674).

### Modified Nucleotides, Nucleosides, Abasic Monomers, and Linkages

The 5′-wing region and the 3′-wing region can each independently comprise 1 to 7 deoxyribonucleotides or “nucleotides”, e.g., 1, 2, 3, 4, 5, 6 or 7 nucleotides. One or more of the nucleotides can be modified (e.g., 1, 2, 3, 4, 5, 6 or 7 of the nucleotides is/are modified). The 5′-wing region and the 3′-wing region can each independently comprise one or more locked nucleosides, 2′-substituted nucleosides, 3′-substituted nucleosides, or abasic monomers. The 5′-wing region and 3′-wing region can comprise one or more (e.g., 1, 2, 3, 4, 5, 6 or 7) locked nucleic acid, 2′-substituted nucleosides, 3′-substituted nucleosides, or abasic monomers. The locked nucleoside can contain a bridge between the 4′ and 2′ position of the sugar wherein the bridges comprise 2 to 4 optionally substituted atoms. For example, the locked nucleic acid is

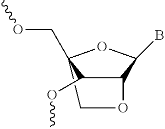

### (LNA),

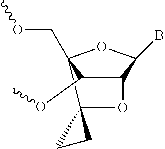

(ScpBNA or “cp”),

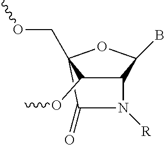

(AmNA when R is H; AmNA(N-Me) when R is CH₃; AmNA(N-alkyl) when R is C₁-C₆ alkyl);

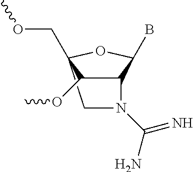

### (GuNA); or

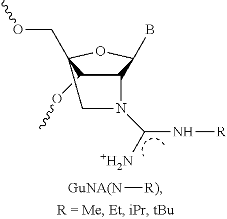

(GuNA(N—R)); wherein B is a nucleobase, R is H or C₁-C₆ alkyl and wherein [symbol] represents a phosphodiester linkage, a phosphorothioate linkage, a mesyl phosphoroamidate linkage, or H. All nucleosides in the 5′-wing region can be locked nucleosides. All nucleosides in the 3′-wing region can be locked nucleosides. The 5′-wing region and 3′-wing regions can each independently contain one or more locked nucleosides, such as one or two nucleosides selected from LNA, ScpBNA, AmNA, and GuNA.

Additionally or alternatively, at least one of the 2′-substituted nucleotides can comprise a nucleotide selected from

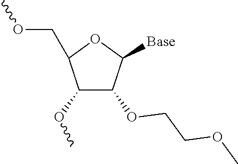

### (2′-O-methoxyethyl (“2′-MOE”)),

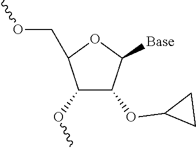

### (2′-O-cyclopropyl (“2′-O-cyp”)),

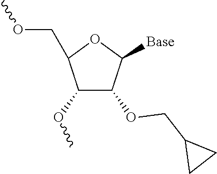

(2′-O-methylcyclopropyl (“2′-O-mcyp”)), and 2′-O-methyl (“2′-OMe”) or any combination thereof, wherein [symbol] represents a phosphodiester linkage, a phosphorothioate linkage, a mesyl phosphoroamidate linkage, or H. Additionally or alternatively, at least one of the 3′-modified nucleosides can comprise a 3′-xylo nucleotide, such as a 2′-OMe-3′-xylo (i.e., “ImX”)

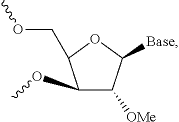

wherein [symbol] represents a phosphodiester linkage, a phosphorothioate linkage, a mesyl phosphoroamidate linkage, or H.

Additionally or alternatively, the 5′-wing region and the 3′-wing region can each comprise at least one ribonucleotide. Additionally or alternatively, the 5′-wing region and the 3′-wing region can each comprise at least one 2,6-diaminopurine (“DAP”),

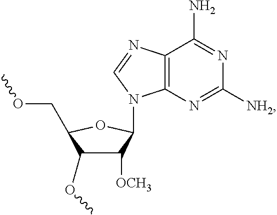

wherein [symbol] represents a phosphodiester linkage, a phosphorothioate linkage, a mesyl phosphoroamidate linkage, or H.

Additionally or alternatively, the 5′-wing region and the 3′-wing region can each comprise at least one of the modified nucleotides including those with the structure of

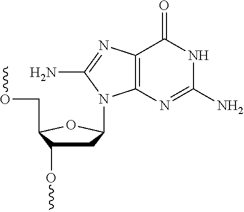

(8-amino-guanosine (“(8nh)G”),

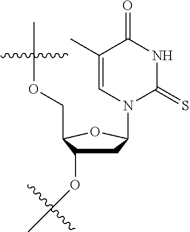

(2-thio-thymine (“(2s)T”)),

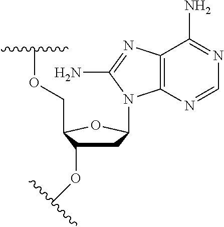

(8-amino-adenosine (“(8nh)A”)),

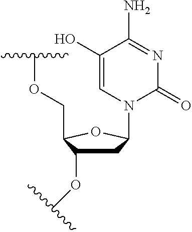

or hydroxy-cytosine (“(5oh)C”), wherein [symbol] represents a phosphodiester linkage, a phosphorothioate linkage, a mesyl phosphoroamidate linkage, or H.

Additionally or alternatively, the 5′-wing region, the 3′-wing region, and/or the central region can each independently comprise at least one abasic monomer. The at least one abasic monomer can have a structure of

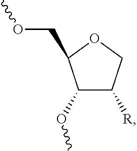

wherein R is H, alkyl (e.g., CH₃), an alkoxy (e.g., O—CH₃ or MOE), O-cyp, or O-mcyp or R can connect to the 4′ of the sugar to form a locked abasic monomer, and wherein [symbol] represents a phosphodiester linkage, a phosphorothioate linkage, a mesyl phosphoroamidate linkage, or H. Additionally or alternatively, the at least one abasic monomer can comprise an abasic monomer selected from

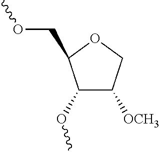

(abasic monomer 1; i.e., a 2′-OMe abasic monomer),

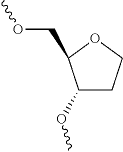

(abasic monomer 2; i.e., a 2′-deoxy abasic monomer),

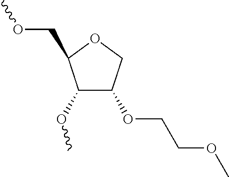

(abasic monomer 3; i.e., a 2′-MOE abasic monomer), and

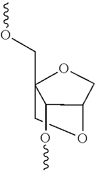

(abasic monomer 4; i.e., locked abasic monomer), or any combination thereof, wherein [symbol] represents a phosphodiester linkage, a phosphorothioate linkage, a mesyl phosphoroamidate linkage, or H.

Additionally or alternatively, the 5′-wing region and the 3′-wing region can each independently comprise 1 to 6 (e.g., 1, 2, 3, 4, 5 or 6) phosphorothioate (“ps”) internucleoside linkages. At least one of the ps internucleoside linkages can comprise a stereo-defined ps internucleoside linkage. The stereo defined ps internucleoside linkage can be an S-ps internucleoside linkage or an R-ps nucleoside linkage.

The 5′-wing region of the disclosed ASOs can include 1 to 7 phosphorothioate-linked locked nucleosides, S phosphorothioate-linked locked nucleosides, R phosphorothioate-linked locked nucleosides, phosphodiester-linked locked nucleosides, or any combination thereof. The 5′-wing region of the disclosed ASOs can include 2 to 6 phosphorothioate-linked 2′ substituted nucleosides, S-phosphorothioate-linked 2′ substituted nucleosides, R-phosphorothioate-linked locked nucleosides, phosphodiester-linked 2′ substituted nucleosides, or any combination thereof. The 5′-wing region of the disclosed ASOs can include 2 to 7 phosphorothioate-linked 3′ substituted nucleosides, S phosphorothioate-linked 3′-modified nucleosides, R phosphorothioate-linked locked nucleosides, phosphodiester-linked 3′ substituted nucleosides, or any combination thereof. The 5′-wing region can further comprise one or more RNA nucleosides or DNA nucleosides, wherein the RNA nucleoside and DNA nucleoside are not locked nucleosides, 2′ substituted nucleosides, or 3′ substituted nucleosides. At least two nucleosides of the 5′-wing region can be linked by a phosphorothioate linker. At least 2, 3, 4, 5, 6 or 7 nucleosides of the 5′-wing region are linked by a phosphorothioate linker.

The 3′-wing region of the disclosed ASOs can include 1 to 7 phosphorothioate-linked locked nucleosides, S phosphorothioate-linked locked nucleosides, R phosphorothioate-linked locked nucleosides, phosphodiester-linked locked nucleosides, or any combination thereof. The 3′-wing region of the disclosed ASOs can include 2 to 7 phosphorothioate-linked 2′ substituted nucleosides, S phosphorothioate-linked 2′ substituted nucleosides, R phosphorothioate-linked locked nucleosides, phosphodiester-linked 2′ substituted nucleosides, or any combination thereof. The 3′-wing region of the disclosed ASOs can include 2 to 7 phosphorothioate-linked 3′ substituted nucleosides, S phosphorothioate-linked 3′ substituted nucleosides, R phosphorothioate-linked locked nucleosides, phosphodiester-linked 3′ substituted nucleosides, or any combination thereof. The 3′-wing region can further comprise one or more RNA nucleosides or DNA nucleosides, wherein the RNA nucleoside and DNA nucleoside are not locked nucleosides, 2′ substituted nucleosides, or 3′ substituted nucleosides. At least two nucleosides of the 3′-wing region can be linked by a phosphorothioate linker. At least 2, 3, 4, 5, 6 or 7 nucleosides of the 3′-wing region are linked by a phosphorothioate linker.

In some embodiments, a mesyl phosphoramidate linkage (“MsPA”) (or an analog thereof), as shown below, can be used alone or in combination with phosphorothioate or phosphodiester linkages. Accordingly, the disclosed ASOs may comprise 1, 2, 3, 4, 5 or more mesyl phosphoramidate linkages (or analogs thereof). The mesyl phosphoramidate linkages can be in the central region or in either or both of the wing regions. For example, in some embodiments, the central gap region can comprise 1, 2, 3, 4, 5, or more contiguous DNA nucleotides, linked by phosphodiester internucleoside linkages or phosphorothioate (“ps”) internucleoside linkages. Additionally or alternatively, the central gap region can comprise 1, 2, 3, 4, 5, or more contiguous DNA nucleotides, linked by mesyl phosphoramidate linkages, or analogs of mesyl phosphoramidate linkages.

The central region can include one or more modified nucleotides, phosphorothioate internucleoside linkages, mesyl phosphoramidate linkages, or any combination thereof. Further, the central region can include one or more modified nucleotides where the central region is capable of inducing RNase H cleavage. The central region can include one or more modified nucleotides where the central region is capable of activating toll-like receptor 8 (TLR8). The central region can include one or more modified nucleotides where the central region is capable of inducing activation of TLR8. The central region can include one or more modified nucleotides where the central region is capable of reducing caspase activity. The central region can include one or more modified nucleotides or one or more phosphorothioate internucleoside linages or one or more mesyl phosphoramidate linkages to reduce toxicity of the ASO and to improve potency of the treatment.

The central region can include one or more modified nucleotides having a modified nucleobase, or no base at all (e.g., an abasic monomer). Abasic monomers included in the central region can be deoxy monomers or comprise an alternative modification at the 2′ position (e.g., a 2′-OMe, or a 2′-MOE). The central region can comprise 5, 6, 7, 8, 9, 10, 11 or 12 contiguous DNA nucleosides. 1, 2, 3, 4, 5, 6, 7, 8, 9, 10, 11, or 12 of the DNA nucleosides in the central gap region can be modified. At least one of the modified nucleotides can comprise 2′-substituted nucleotides, including, but not limited, to a structure of 2′-O-cyp, 2′-O-mcyp, 2′-OMe, 2′-MOE or

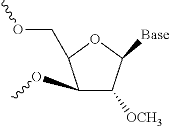

(2′-O-methyl-3′-xylo (“2′-OMe-3′-xylo”)), wherein [symbol] represents a phosphodiester linkage, a phosphorothioate linkage, or a mesyl phosphoroamidate linkage. Additionally or alternatively, at least one of the modified nucleotides can comprise a structure of

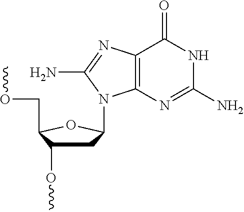

(8-amino-guanosine (“(8nh)G”),

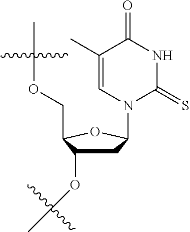

(2-thio-thymine (“(2s)T”)),

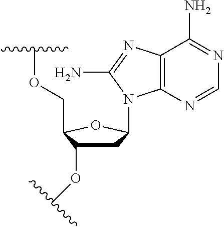

(8-amino-adenosine (“(8nh)A”)), or

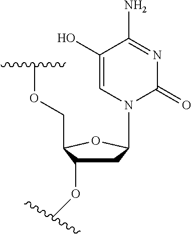

(5-hydroxy-cytosine (“(5oh)C”), wherein [symbol] represents a phosphodiester linkage, a phosphorothioate linkage, or a mesyl phosphoroamidate linkage. Additionally or alternatively, the central region can comprise at least one locked nucleoside, including, but not limited to, LNA, ScpBNA, AmNA, or GuNA. Additionally or alternatively, the central gap region can comprise at least one ribonucleotide.

The central region can comprise at least one abasic monomer (e.g., 1, 2, 3, 4, or more abasic monomers). The at least one abasic monomer can have a structure of

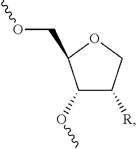

wherein R is H, alkyl (e.g., CH₃), an alkoxy (e.g., O—CH₃ or MOE), O-cyp, or O-mcyp or R can connect to the 4′ of the sugar to form a locked abasic monomer, and wherein [symbol] represents a phosphodiester linkage, a phosphorothioate linkage, or a mesyl phosphoroamidate linkage. Additionally or alternatively, the at least one abasic monomer can comprise an abasic monomer selected from

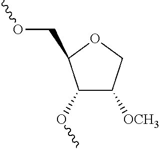

(abasic monomer 1; i.e., a 2′-OMe abasic monomer),

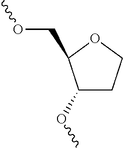

(abasic monomer 2; i.e., a 2′-deoxy abasic monomer),

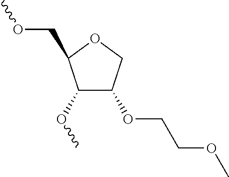

(abasic monomer 3; i.e., a 2′-MOE abasic monomer), and

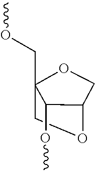

(abasic monomer 4; i.e., locked abasic monomer), or any combination thereof, wherein [symbol] represents a phosphodiester linkage, a phosphorothioate linkage, or a mesyl phosphoroamidate linkage. The position of the abasic monomer within the central region can vary. For example, the central region may comprise an abasic monomer at position 1, 2, 3, 4, 5, 6, 7, 8, 9, or 10 of the central region (which may be defined by DNA residues) relative to the 5′ end of the central region. Depending on the size of the 5′ wing, position 1, 2, 3, 4, 5, 6, 7, 8, 9, or 10 of the central region may correspond to different positions within the ASO. For example, many of the ASOs disclosed herein comprise 5 nucleotides in the 5′ wing, and therefore positions 1, 2, 3, 4, 5, 6, 7, 8, 9, and 10 of the central region correspond to positions 6, 7, 8, 9, 10, 11, 12, 13, 14, and 15 of the ASO, respectively.

Additionally or alternatively, the central region can comprise 1 to 11 (e.g., 1, 2, 3, 4, 5, 6, 7, 8, 9, 10 or 11) phosphorothioate (“ps”) internucleoside linkages. At least one of the ps internucleoside linkages can comprise a stereo-defined ps internucleoside linkage. The stereo defined ps internucleoside linkage can be an S-ps internucleoside linkage or an R-ps nucleoside linkage. Additionally or alternatively, the central region can comprise 1 to 11 (e.g., 1, 2, 3, 4, 5, 6, 7, 8, 9, 10 or 11) mesyl phosphoramidate linkages. The central region can comprise at least one ps internucleoside linkage and at least one mesyl phosphoramidate linkage.

The central region of the disclosed ASOs can comprise at least 5 contiguous phosphorothioate-linked DNA nucleosides. At least 2, 3, 4, 5, or 6 nucleotides of the central region can be linked by a phosphorothioate linker. 1, 2, 3, 4, 5, or 6 of the nucleotides of the central region can be linked by a mesyl phosphoramidate linkage. The central region of the ASO may not include mesyl phosphoramidate linkages between the nucleotides of the central region. A DNA nucleoside of the central region can be linked to a nucleoside of the 5′-wing region by a phosophorothioate linker. A DNA nucleoside of the central region can be linked to a nucleoside of the 3′-wing region by a phosphorothioate linker. The central region can comprise 8 to 12 contiguous DNA nucleotides.

For the purposes of the present disclosure, the disclosed ASOs can comprise at least 1, at least 2, at least 3, at least 4, or at least 5 or more modifications of 2′-MOE, 2′-O-cyp, 3′-xylo, 2′-O-mcyp, 2′-OMe, 2′-OMe-3′-xylo, or any combination thereof. Additionally or alternatively, the disclosed ASOs can comprise at least 1, at least 2, at least 3, at least 4, or at least 5 or more modified nucleobases of (8nh)G, (2s)T, (8nh)A, (5oh)C, or a combination thereof. Additionally or alternatively, the disclosed ASOs can comprise at least 1, at least 2, at least 3, at least 4, or at least 5 or more abasic monomers. In some embodiments, the disclosed ASOs can comprise 1, 2, 3, 4, or 5 or more abasic monomers. Additionally or alternatively, the disclosed ASOs can comprise 1, 2, 3, 4, 5, 6, 7, 8, 9, 10, 11, 12, 13, 14, 15 or more ps internucleoside linkages, S-ps internucleoside linkages, R-ps internucleoside linkages, mesyl phosphoramidate linkages, or any combination thereof. In some embodiments, the modifications can be in the gap region or the wing region.

The gapmer ASO compounds of the disclosure include compounds of formula (I): A′-B′—C′, wherein A′ and C′ each can independently comprise 2 to 7 nucleotides, with one or more being a modified nucleoside, and B′ can comprise 6 or more DNA nucleosides and/or abasic monomers linked by phosphodiester, phosphorothioate, or mesyl phosphoramidate linkages. B′ can comprise one or more modified DNA nucleosides or abasic monomers (e.g., abasic monomer 1, abasic monomer 2, abasic monomer 3, or abasic monomer 4). The modified DNA nucleoside can be selected from locked nucleosides, 2′ substituted nucleosides, or 3′ substituted nucleoside. The locked nucleosides can be selected from LNA, ScpBNA, AmNA, and GuNA. The 2′ substituted nucleosides can be selected from 2′-MOE, 2′-O-cyp, 2′-O-mcyp, 2′-OMe, and 2′—OMe-3′-xylo. The 3′ substituted nucleoside can be selected from 3′-xylo. Additional modified nucleosides include, but are not limited to, (8nh)G, (2s)T, (8nh)A, and (5oh)C.

The number of nucleotides and/or nucleosides in A′, B′, and C′ can be selected from the following group (A′: B′: C′): (6:5:7), (7:5:6), (7:5:7), (5:6:7), (6:6:6), (6:6:7), (7:6:5), (7:6:6), (7:6:7), (4:7:7), (5:7:6), (5:7:7), (6:7:5), (6:7:6), (6:7:7), (7:7:4), (7:7:5), (7:7:6), (7:7:7), (3:8:7), (4:8:6), (4:8:7), (5:8:5), (5:8:6), (5:8:7), (6:8:4), (6:8:5), (6:8:6), (6:8:7), (7:8:3), (7:8:4), (7:8:5), (7:8:6), (7:8:7), (2:9:7), (3:9:6), (3:9:7), (4:9:5), (4:9:6), (4:9:7), (5:9:4), (5:9:5), (5:9:6), (5:9:7), (6:9:3), (6:9:4), (6:9:5), (6:9:6), (6:9:7), (7:9:2), (7:9:3), (7:9:4), (7:9:5), (7:9:6), (7:9:7), (2:10:6), (2:10:7), (3:10:5), (3:10:6), (3:10:7), (4:10:4), (4:10:5), (4:10:6), (4:10:7), (5:10:3), (5:10:4), (5:10:5), (5:10:6), (5:10:7), (6:10:2), (6:10:3), (6:10:4), (6:10:5), (6:10:6), (6:10:7), (7:10:2), (7:10:3), (7:10:4), (7:10:5), (7:10:6), (2:11:6), (2:11:7), (3:11:5), (3:11:6), (3:11:7), (4:11:4), (4:11:5), (4:11:6), (4:11:7), (5:11:3), (5:11:4), (5:11:5), (5:11:6), (5:11:7), (6:11:2), (6:11:3), (6:11:4), (6:11:5), (6:11:6), (7:11:2), (7:11:3), (7:11:4), (7:11:5), (2:12:4), (2:12:5), (2:12:6), (2:12:7), (3:12:3), (3:12:4), (3:12:5), (3:12:6), (3:12:7), (4:12:2), (4:12:3), (4:12:4), (4:12:5), (4:12:6), (4:12:7), (5:12:2), (5:12:3), (5:12:4), (5:12:5), (5:12:6), (6:12:1), (6:12:2), (6:12:3), (6:12:4), and (6:12:5).

The 5′-wing region can comprise one or more locked nucleosides or 2′-substituted nucleosides. The 3′-wing region can comprise one or more locked nucleosides or 2′-substituted nucleosides. The central region can comprise one or more locked nucleosides or 2′-substituted nucleosides. The 5′-wing region, the 3′-wing region, the central gap region, or a combination thereof can comprise one or more (e.g., 1, 2, 3, 4, 5, 6, 7, 8, 9, 10, 11, 12, 13, 14, 15) locked nucleosides or 2′-substituted nucleosides. The locked nucleoside can contain a bridge between the 4′ and the 2′ position of the sugar wherein the bridges can comprise 2 to 4 optionally substituted atoms. Exemplary locked nucleosides include those discussed above. All nucleosides in the 5′-wing region can be locked nucleosides. The 5′-wing region can contain a locked nucleoside, such as one or two or more nucleosides selected from LNA, ScpBNA, AmNA, and GuNA. All nucleosides in the 3′-wing region can be locked nucleosides. The 3′-wing region can contain LNA and one or two or more nucleosides selected from ScpBNA, AmNA, and GuNA. Other nucleotides are included in PCT/JP2010/068409, US2012/0208991, PCT/JP2013/075370, US2015/0266917, PCT/JP2015/054308, US2017/0044528, PCT/JP2018/006061, US2020/0056178, PCT/JP2018/006062, and/or US2020/0055890, which are incorporated by reference in their entirety. One or more nucleotides in the 5′-wing and/or the 3′ wing region can each independently comprise one or more modified nucleosides.

The 5′-wing region can include one modified nucleotide (e.g., a 2′ substituted nucleoside or 3′ substituted nucleoside) at the first, second, third, fourth, fifth, sixth or seventh nucleoside position (from the 5′ end of ASO). The 5′-wing region can include more than one modified nucleotide at the first, second, third, fourth, fifth, sixth or seventh nucleoside position (from the 5′ end of ASO). The 5′-wing region can include seven modified nucleotides at the first, second, third, fourth, fifth, sixth and seventh nucleoside positions (from the 5′ end of ASO). The seven modified nucleotides can be 2′-O-methoxyethyl. The 5′-wing region can include one or more modified nucleotide of (8nh)G, (2s)T, (8nh)A, or (5oh)C at the first, second, third, fourth, fifth, sixth or seventh nucleoside position (from the 5′ end of ASO).

The 3′-wing region can include one modified nucleotide (e.g., a 2′ substituted nucleoside or 3′ substituted nucleoside) at the first, second, third, fourth, fifth, sixth or seventh nucleoside position (from the 5′ end of 3′-wing region). The 3′-wing region can include more than one modified nucleotide at the first, second, third, fourth, fifth, sixth or seventh nucleoside position (from the 5′ end of 3′-wing region). The 3′-wing region can include seven modified nucleotides at the first, second, third, fourth, fifth, sixth and seventh nucleoside positions (from the 5′ end of 3′-wing region). The seven modified nucleotides can be 2′-O-methoxyethyl. The modified nucleotides at the first, second, third, fourth, fifth and sixth nucleotide positions can be 2′-O-methoxyethyl, and the modified nucleotide at the seventh position can be 2′-O-cyclopropyl. The 3′-wing region can include one modified nucleotide of (8nh)G, (2s)T, (8nh)A, or (5oh)C at the first, second, third, fourth, fifth sixth or seventh nucleoside position (from the 5′ end of 3′-wing region).

The 5′-wing region can comprise one or more abasic monomers. The 3′-wing region can comprise one or more abasic monomers. The central region can comprise one or more abasic monomers. The 5′-wing region, the 3′-wing region, the central gap region, or a combination thereof can comprise one or more (e.g., 1, 2, 3, 4, 5, 6, 7, 8, 9, 10, 11, 12, 13, 14, 15) abasic monomers. Exemplary abasic monomers include those discussed above (i.e., abasic monomers 1-4). All positions in the 5′-wing region can be abasic monomers. The 5′-wing region can contain an abasic monomer, such as one or two or more abasic monomers selected from any one of abasic monomer 1, abasic monomer 2, abasic monomer 3, abasic monomer 4, or any combination thereof. The 3′-wing region can contain one or two or more abasic monomers selected from comprise any one of abasic monomer 1, abasic monomer 2, abasic monomer 3, abasic monomer 4, or any combination thereof.

The 3′-wing region can include one abasic monomer at the first, second, third, fourth, fifth, sixth or seventh position (from the 5′ end of 3′-wing region). The 3′-wing region can include more than one abasic monomers at the first, second, third, fourth, fifth, sixth or seventh nucleoside position (from the 5′ end of 3′-wing region). The abasic monomer can comprise any one of abasic monomer 1, abasic monomer 2, abasic monomer 3, abasic monomer 4, or any combination thereof.

The central gap region can include one or more modified nucleotide having a modified nucleobase. For example, the central region can include at least 1, at least 2, at least 3, at least 4, or at least 5 or more locked nucleosides, 2′ substituted nucleosides, 3′ substituted nucleosides, or a combination thereof. Additionally or alternatively, the central region can include one or more modified nucleosides including locked nucleosides, including but not limited to LNA, ScpBNA, AmNA, and GuNA, or one or more abasic monomers or any combination thereof. The central region can include one modified nucleotide (e.g., a 2′-substituted nucleoside such as 2′-OMe-3′xylo) at the first, second, third, or fourth gap nucleoside position (from the 5′ end of the central region). The modified nucleotide can be at the third gap nucleoside position (from the 5′ end of the central region). The modified nucleotide can be at the first gap nucleoside position (from the 5′ end of the central region). The central region can include one modified nucleotide of (8nh)G, (2s)T, (8nh)A, or (5oh)C at the second, third, fourth, fifth, sixth, seventh, eighth, or ninth nucleoside position (from the 5′ end of the central region). Other modified nucleotides include those in PCT/JP2018/006061 and US2020/0056178, which is incorporated by reference in its entirety.

The central region of an ASO can comprise at least 5 contiguous phosphorothioate-linked DNA nucleosides, at least 5 contiguous phosphodiester-linked DNA nucleosides, at least 5 contiguous mesyl phosphoramidate linked-DNA nucleosides, or a combination thereof. At least 1, 2, 3, 4, 5, 6, 7, 8, 9, 10, 11, or 12 nucleosides of the central gap region are linked by a phosphorothioate linker, a phosphodiester linker, a mesyl phosphoramidate linker, or a combination thereof. The central region can comprise 8 to 12 contiguous phosphorotioate-linked DNA nucleosides, 8 to 12 contiguous phosphodiester-linked DNA nucleosides, 8 to 12 contiguous mesyl phosphoramidate linked DNA nucleosides, or a combination thereof. At least one of the phosphorothioate linkers can be an S phosphorothioate linker. The S phosphorothioate linker can be linking the nucleosides at the second and third nucleoside positions (from the 5′ end of central region). The S phosphorothioate linked can be linking the fourth and fifth nucleoside positions (from the 5′ end of central region). At least one of the phosphorothioate linkers can be an R phosphorothioate linker. A DNA nucleoside of the central gap region is linked to a nucleoside of the 5′-wing region by a phosphorothioate linker or phosphodiester linker. A DNA nucleoside of the central gap region is linked to a nucleoside of the 3′-wing region by a phosphorothioate linker or phosphodiester linker.

The central gap region can include one or more abasic monomers. For example, the central region can include at least 1, at least 2, at least 3, at least 4, or at least 5 or more abasic monomers. Additionally or alternatively, the central region can include one or more abasic monomers, including but not abasic monomer 1, abasic monomer 2, abasic monomer 3, abasic monomer 4, or any combination thereof. The central region can include one abasic monomer at the first, second, third, fourth, fifth sixth, seventh, eighth, nineth, or tenth gap position (from the 5′ end of the central region). In some embodiments, the abasic monomer can be at the fifth gap nucleoside position (from the 5′ end of the central region). In some embodiments, the abasic monomer can be at the sixth gap nucleoside position (from the 5′ end of the central region). The central region can include one abasic monomer of abasic monomer 1, abasic monomer 2, abasic monomer 3, abasic monomer 4, or any combination thereof at the first, second, third, fourth, fifth, sixth, seventh, eighth, ninth, or tenth nucleoside position (from the 5′ end of the central region).

The central region of an ASO can comprise at least 5 contiguous phosphorothioate-linked abasic monomers, at least 5 contiguous phosphodiester-linked abasic monomers, at least 5 contiguous mesyl phosphoramidate linked-abasic monomers, or a combination thereof. At least 1, 2, 3, 4, 5, or 6 abasic monomers of the central gap region are linked by a phosphorothioate linker, a phosphodiester linker, a mesyl phosphoramidate linker, or a combination thereof. The central region can comprise 8 to 12 contiguous phosphorotioate-linked abasic monomers, 8 to 12 contiguous phosphodiester-linked abasic monomers, 8 to 12 contiguous mesyl phosphoramidate linked abasic monomers, or a combination thereof. At least one of the phosphorothioate linkers can be an S phosphorothioate linker. The S phosphorothioate linker can be linking the abasic monomers at the second and third nucleoside positions (from the 5′ end of central region). The S phosphorothioate linked can be linking the fourth and fifth abasic monomer positions (from the 5′ end of central region). At least one of the phosphorothioate linkers can be an R phosphorothioate linker. An abasic monomer of the central gap region can be linked to a nucleoside or an abasic monomer of the 5′-wing region by a phosphorothioate linker or phosphodiester linker. An abasic monomer of the central gap region can be linked to a nucleoside or an abasic monomer of the 3′-wing region by a phosphorothioate linker or phosphodiester linker.

### Antisense Oligonucleotide Sequence and Target RNA Sequence

The ASO can be complementary or hybridize to a viral target RNA sequence of HBV or in an S region or X region of HBV. The viral target can, e.g., begin at the 5′-end of the target site in acc. KC315400.1 (genotype B, “gt B”), or in any one of genotypes A, C, D, E, F, G, H or I. The skilled person would understand the HBV position, e.g., as described in Wing-Kin Sung, et al., Nature Genetics 44:765 (2012).

The ASO can be complementary or hybridize to a viral target RNA sequence that can comprise, consist of, or consist essentially of at least 5, 6, 7, 8, 9, 10, 11, or 12 contiguous nucleotides within positions 1575-1610 of SEQ ID NO: 1, positions 1575-1606 of SEQ ID NO: 1, or 1579-1606 of SEQ ID NO: 1. The ASO can be complementary or hybridize to a viral target RNA sequence that can comprise, consist of, consist essentially of 5 to 15, 5 to 14, 5 to 13, 5 to 12, 5 to 11, 5 to 10, 5 to 9, 5 to 8, 6 to 15, 6 to 14, 6 to 13, 6 to 12, 6 to 11, 6 to 10, 7 to 15, 7 to 14, 7 to 13, 7 to 12, or 7 to 11 contiguous nucleotides within positions 1575-1610 of SEQ ID NO: 1, positions 1575-1606 of SEQ ID NO: 1, or 1579-1606 of SEQ ID NO: 1. The ASO can be complementary or hybridize to a viral target RNA sequence that can comprise, consist of, or consist essentially of at least 5, 6, 7, 8, 9, 10, 11, 12, 13, 14, 15, 16, 17, 18, 19, 20, 21, 22, or 23 contiguous nucleotides within positions 1575-1610 of SEQ ID NO: 1, positions 1575-1606 of SEQ ID NO: 1, or 1579-1606 of SEQ ID NO: 1. The ASO can be perfectly complementary to the viral target RNA sequence, or there can be less than or equal to 5, 4, 3, 2, or 1 mismatches between the ASO and the viral target RNA sequence. There can be less than or equal to 2 mismatches between the ASO and the viral target RNA sequence. There can be less than or equal to 1 mismatch between the ASO and the viral target RNA sequence. The mismatch can be in the wing region of the ASO. The mismatch can be in the 5′-wing region of the ASO. The mismatch can be in the 3′-wing region of the ASO. The mismatch can be in the central gap region of the ASO.

The central region can be complementary or hybridize to a viral target RNA sequence that can comprise, consist of, or consist essentially of at least 5, 6, 7, 8, 9, 10, 11, 12, 13, 14, or 15 contiguous nucleotides within positions 1570-1610 of SEQ ID NO: 1, positions 1575-1605 of SEQ ID NO: 1, or 1580-1605 of SEQ ID NO: 1. The central region can be complementary or hybridize to a viral target RNA sequence that can comprise, consist of, or consist essentially of 5 to 15, 5 to 14, 5 to 13, 5 to 12, 5 to 11, 5 to 10, 5 to 9, 5 to 8, 6 to 15, 6 to 14, 6 to 13, 6 to 12, 6 to 11, 6 to 10, 7 to 15, 7 to 14, 7 to 13, 7 to 12, or 7 to 11 contiguous nucleotides within positions 1570-1610 of SEQ ID NO: 1, positions 1575-1605 of SEQ ID NO: 1, or 1580-1605 of SEQ ID NO: 1. The central region can be perfectly complementary to the viral target RNA sequence. Alternatively, there can be less than or equal to 5, 4, 3, 2, or 1 mismatch between the central region and the viral target RNA sequence. There can be less than or equal to 2 mismatches between the central region and the viral target RNA sequence. There can be less than or equal to 1 mismatch between the central region and the viral target RNA sequence.

The ASO can comprise a nucleotide sequence that is at least 90%, at least 91%, at least 92%, at least 93%, at least 94%, at least 95%, at least 96%, at least 97%, at least 98%, at least 99%, or 100% identical to a nucleotide sequence selected from the sequences listed in Table 1 or Table 2.

The ASOs of the disclosure can have a sequence that differs from an ASO of Table 2 by one nucleoside. The ASOs of the disclosure can have a sequence that differs from an ASO of Table 2 by two nucleosides. The ASOs of the disclosure can have a sequence that differs from the ASO of Table 2 by three nucleosides. The ASOs of the disclosure can have a sequence that differs from an ASO of Table 2 by four nucleosides. In some embodiments, nucleoside differences may comprise abasic monomers.

The ASOs of the disclosure can have a sequence of Table 2, but one T in the central region is replaced by (2s)T, one C in the central region is replaced by (5oh)C, and/or one A is replaced by (8nh)A in the central region. The ASOs of the disclosure can have a sequence of Table 2, but with one or two ScpBNA, AmNA, or GuNA in the 5′-wing portion. The ASOs of the disclosure can have a sequence of Table 2, but with one or two ScpBNA, AmNA, or GuNA in the 3′-wing portion. The ASO can comprise a nucleotide sequence that is at least 90%, at least 91%, at least 92%, at least 93%, at least 94%, at least 95%, at least 96%, at least 97%, at least 98%, at least 99%, or 100% identical to a nucleotide sequence of any one of SEQ ID NOs: 3-320, 353-404, and 446-1344.

ASOs of particular interest, due to observed activity, include but are not limited to, ASO-676 (SEQ ID NO: 697), ASO-677 (SEQ ID NO: 698), ASO-1037 (SEQ ID NO: 1058), ASO-707 (SEQ ID NO: 728), ASO-1192 (SEQ ID NO: 1213), ASO-651 (SEQ ID NO: 672), ASO-1166 (SEQ ID NO: 1187), ASO-1181 (SEQ ID NO: 1202), ASO-1179 (SEQ ID NO: 1200), and ASO-962 (SEQ ID NO: 983). In some embodiments, the ASO is selected from ASO-676 (SEQ ID NO: 697), ASO-677 (SEQ ID NO: 698), ASO-1037 (SEQ ID NO: 1058), ASO-707 (SEQ ID NO: 728), and ASO-1192 (SEQ ID NO: 1213). In some embodiments, the ASO is selected from ASO-651 (SEQ ID NO: 672), ASO-1166 (SEQ ID NO: 1187), ASO-1181 (SEQ ID NO: 1202), and ASO-1179 (SEQ ID NO: 1200). In some embodiments, the ASO is ASO-962 (SEQ ID NO: 983).

The disclosed ASO can decrease expression of a target RNA sequence (e.g., a target gene) by recruiting RNase H to cleave and degrade the RNA transcript of the target RNA sequence, lowering RNA levels and thereby lowering levels of the protein encoded by the target RNA sequence. The disclosed ASO can act to activate toll-like receptor 8 (TLR8). TLR8 is associated with production of IL-12, a proinflammatory cytokine that plays a role in stimulating cell-mediated immunity and may play a role in achieving sustained control of HBV replication.

### Conjugates

The ASOs disclosed herein can further include one or more conjugates. The conjugate may be attached to the 5′ end and/or the 3′ end of the ASO via covalent attachment, such as to a nucleotide. The conjugate can be covalently attached via a linker to the ASO. The conjugate can be attached to nucleobases, sugar moieties, or internucleoside linkages of the ASO molecules of the disclosure.

The type of conjugate or ligand used and the extent of conjugation of the ASO molecules of the disclosure can be evaluated, for example, for improved pharmacokinetic profiles, bioavailability, and/or stability of ASO molecules while at the same time maintaining the ability of the ASO to mediate functional activity. A conjugate or ligand may alter distribution, targeting, or lifetime of an ASO molecule into which it is incorporated. A conjugate or ligand may provide an enhanced affinity for a selected target (e.g., molecule, cell, or cell type), compartment (e.g., cellular or organ compartment), tissue, organ, or region of the body, as, e.g., compared to a molecule lacking such a conjugate or ligand.

A conjugate or ligand can include a naturally occurring substance or a recombinant or synthetic molecule. Non-limiting examples of conjugates and ligands include serum proteins (e.g., humans serum albumin, low-density lipoprotein, globulin), cholesterol moieties, vitamins (e.g., biotin, vitamin E, vitamin B12), folate moieties, steroids, bile acids (e.g., cholic acid), fatty acids (e.g., palmitic acid, myristic acid), sugars (e.g., mannose), carbohydrate (e.g., a dextran, pullulan, chitin, chitosan, inulin, cyclodextrin, hyaluronic acid, or N-acetyl-galactosamine [GalNAc]), glycosides, phospholipids, antibodies or binding fragments thereof (e.g., an antibody or binding fragment that targets the ASO to a specific cell type such as liver), dyes, intercalating agents (e.g., acridines), cross-linkers (e.g., psoralene, mitomycin C), porphyrins (e.g., TPPC4, texaphyrin, Sapphryin), polycyclic aromatic hydrocarbons (e.g., phenazine, dihydrophenazine), artificial endonucleases (e.g., EDTA), lipophilic molecules (e.g., cholesterol, tocopherol, long fatty acids [e.g., docosanoic, palmitoyl, docosahexanoic], cholic acid, adamantane acetic acid, 1-pyrene butyric acid, dihydrotestosterone, 1,3-BisO(hexadecyl)glycerol, geranyloxyhexyl group, hexadecylglycerol, borneol, menthol, 1,3-propanediol, heptadecyl group, 03-(oleoyl) lithocholic acid, 03-(oleoyl) cholenic acid, dimethoxytrityl, or phenoxazine), peptides (e.g., antennapedia peptide, Tat peptide, RGD peptides), alkylating agents, polymers, such as polyethylene glycol (PEG) (e.g., PEG-40K), poly amino acids, polyamines (e.g., spermine, spermidine), alkyls, substituted alkyls, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

The conjugate or ligan may include a carbohydrate. Carbohydrates include, but are not limited to, sugars (e.g., monosaccharides, disaccharides, trisaccharides, tetrasaccharides, and oligosaccharides containing from about 4, 5, 6, 7, 8, or 9 monosaccharide units) and polysaccharides, such as starches, glycogen, cellulose and polysaccharide gums. The carbohydrate incorporated into the ligand may be a monosaccharide selected from a mannose, pentose, hexose, or heptose and di- and tri-saccharides including such monosaccharide units.

The carbohydrate incorporated into the conjugate or ligand can be an amino sugar, such as galactosamine, glucosamine, N-acetyl-galactosamine (GalNAc), and N-acetyl-glucosamine. The conjugate or ligand can be N-acetyl-galactosamine and derivatives thereof. Non-limiting examples of GalNAc- or galactose-containing ligands that can be incorporated into the siRNAs of the disclosure are described in WO 2020/243490; WO 2020/097342; WO 2021/119325; WO 2021/173812; WO 2021/173811; WO 2021/178885; Sig. Transduct. Target Ther. 5 (101), 1-25, 2020; ACS Chem. Biol. 10 (5), 1181-1187, 2015; J. Am. Chem. Soc. 136 (49), 16958-16961, 2014; Nucleic Acids Res. 42 (13), 8796-8807, 2014; Molec. Ther. 28 (8), 1759-1771, 2020; and Nucleic Acid Ther. 28 (3), 109-118, 2018, all of which are hereby incorporated herein by reference in their entireties.

The conjugate or ligand can be attached or conjugated to the ASO molecule directly or indirectly. For example, the conjugate can be covalently attached directly to the ASO molecule, or the conjugate can be covalently attached via a linker to the ASO molecule. The conjugate can be attached to nucleobases, sugar moieties, or internucleoside linkages of the ASO molecule of the disclosure. The conjugate or ligand may be attached to the 5′ end and/or to the 3′ end of the ASO molecule. The conjugate can be covalently attached to the 5′ end of the ASO. The conjugate can be covalently attached to the 3′ end of the ASO. The conjugate can be attached to the 5′ terminal nucleotide of the ASO or to the 3′ terminal nucleotide of the ASO.

The conjugate or ligand covalently attached to the ASO molecule can be a GalNAc derivative. The GalNAc derivative can be attached to the 5′ end and/or to the 3′ end of the ASO molecule. The GalNAc can be attached to the 3′ end of the ASO molecule. The GalNAc can be attached to the 5′ end of the ASO molecule.

The conjugate or ligand can be a GalNAc derivative comprising 1, 2, 3, 4, 5, or 6 monomeric GalNAc units. The conjugate or ligand can be a GalNAc derivate having one monomeric GalNAc unit. The conjugate or ligand can be a GalNAc derivative having two monomeric GalNAc units. The conjugate or ligand can be a GalNAc derivative having three monomeric GalNAc units. The conjugate or ligand can be a GalNAc derivative having four monomeric GalNAc units. The conjugate or ligand can be a GalNAc derivative having five monomeric GalNAc units. The conjugate or ligand can be a GalNAc derivative having six monomeric GalNAc units. Various amounts of monomeric GalNAc units are attached at the 5′ end and the 3′ end of the ASO molecule. 1, 2, 3, 4, 5, or 6 monomeric GalNAc units can be attached to the 5′ end of the ASO molecule. 1, 2, 3, 4, 5, or 6 monomeric GalNAc units can be attached to the 3′ end of the ASO molecule. The same number of monomeric GalNAc units can be attached at both the 5′ end and the 3′ end of the ASO molecule. Different numbers of monomeric GalNAc units can be attached at the 5′ end and the 3′ end of the ASO molecule.

The GalNAc can be attached to the 3′ end of the ASO via 1, 2, 3, 4, or 5 or more linkers. The GalNAc can be attached to the 5′ end of the ASO via 1, 2, 3, 4, or 5 or more linkers. The one or more linkers can be independently selected from the group consisting of a phosphodiester (p or po) linker, a phosphorothioate (ps) linker, mesyl phosphoramidate (Ms) linker, phosphoramidite-containing HEG linker, triethylene glycol (TEG) linker, and/or phosphorodithioate linker. The one or more linker(s) can be independently selected from the group consisting of: p-(PS)2, (PS)2-p-TEG-p, (PS)2-p-HEG-p, and (PS)2-p-(HEG-p)2.

The GalNAc can be of Formula (VI):

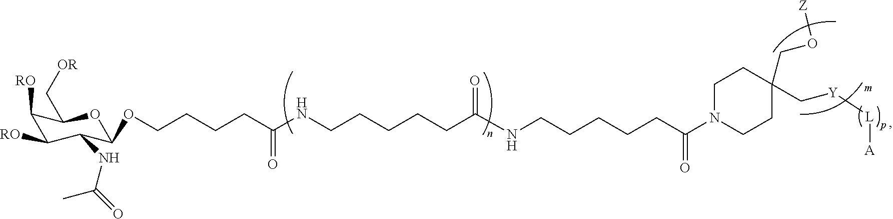

wherein m is 1, 2, 3, 4, or 5; each n is independently 1 or 2; p is 0 or 1; each R is independently H or a first protecting group; each Y is independently selected from —O—P(═O)(SH)—, —O—P(═O)(O)—, —O—P(═O)(OH)—, —O—P(S)S—, and —O—; Z is H or a second protecting group; either L is a linker or L and Y in combination are a linker; and A is H, OH, a third protecting group, an activated group, or an oligonucleotide. The first protecting group can be acetyl. The second protecting group can be trimethoxytrityl (TMT). The activated group can be a phosphoramidite group. The phosphoramidite group can be a cyanoethoxy N,N-diisopropylphosphoramidite group. The linked can be a C6-NH₂ group. A can be the ASO molecule. m can be 3. R can be H, Z can be H, and n can be 1. R can be H, Z can be H, and n can be 2.

The GalNAc can be of Formula (VII):

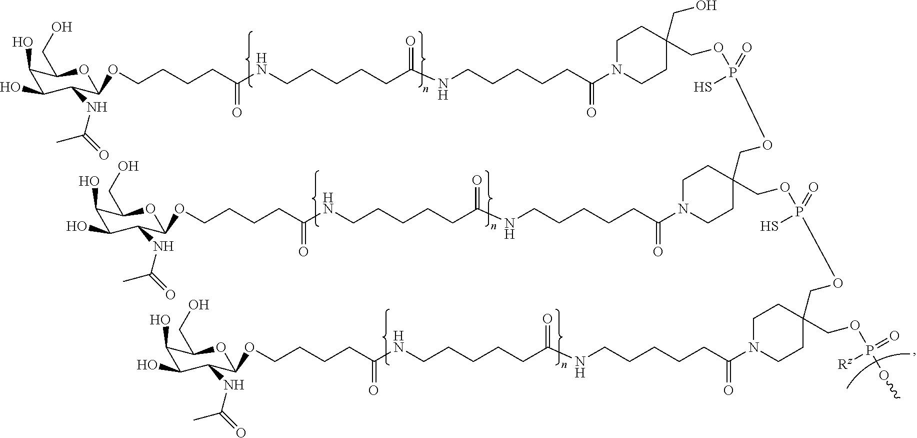

wherein R^{z} is OH or SH; and each n is independently 1 or 2. The GalNAc targeting ligand can include 1, 2, 3, 4, 5, or 6 GalNAc units. The conjugated moiety may be a GalNAc selected from GalNAc2, GalNAc3, GalNAc4 (the GalNAc of Formula VII, wherein n=1 and R^{z}=OH), GalNAc5, and GalNAc6. The GalNAc may be GalNAc amidite,

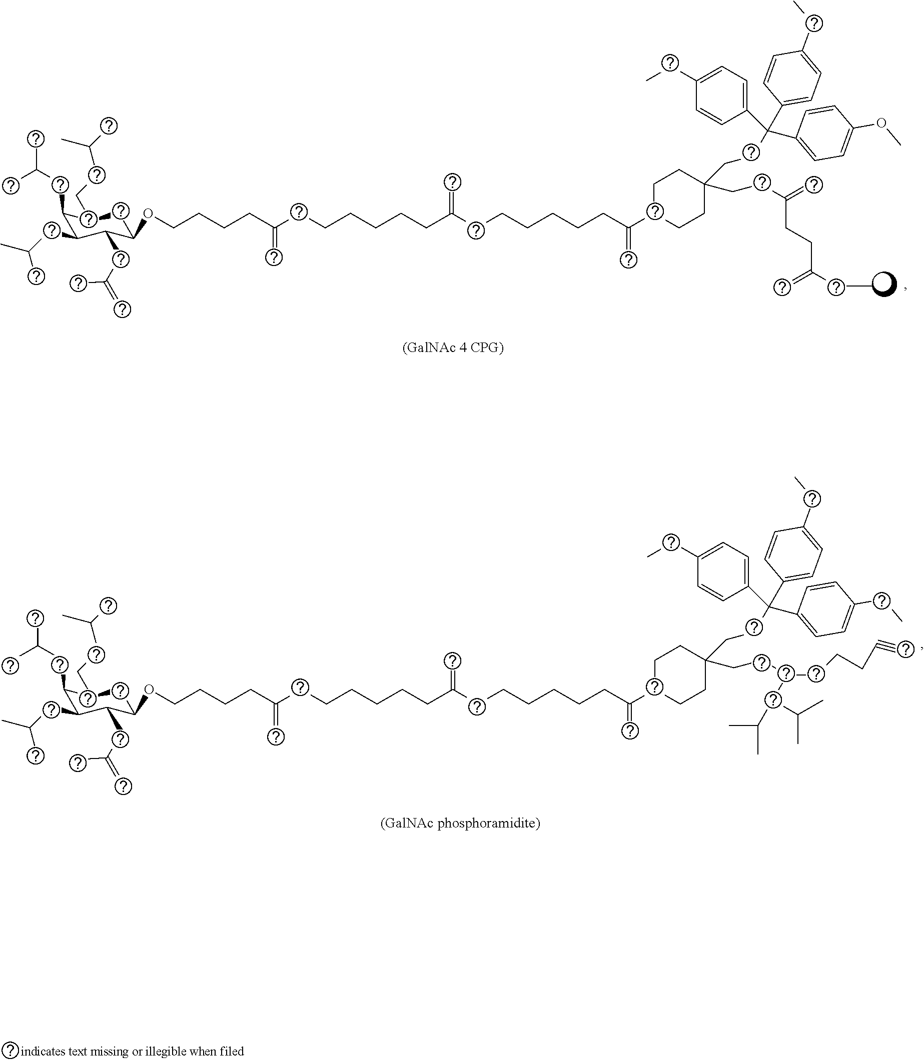

or GalNAc4-ps-GalNAc4-ps-GalNAc4. GalNAc3, GalNAc4, GalNAc5, and GalNAc6 may be conjugated to an ASO disclosed herein during synthesis with 1, 2, or 3 moieties. Further, GalNAc moieties, such as GalNAc1 and GalNAc2, can be used to form 5′ and 3′ GalNAc using post-synthesis conjugation.

GalNAc building blocks
GalNAc-3 phosphoramidite 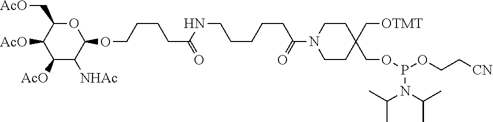
GalNAc-4 phosphoramidite 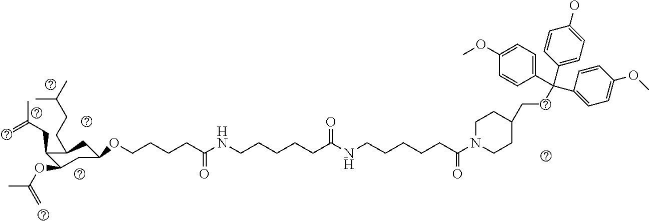
GalNAc-5 phosphoramidite 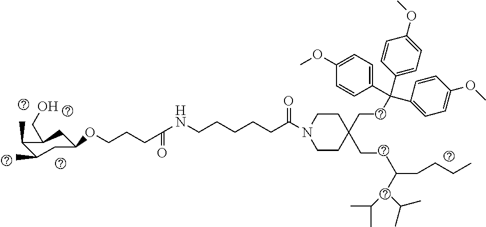
GalNAc-6 phosphoramidite 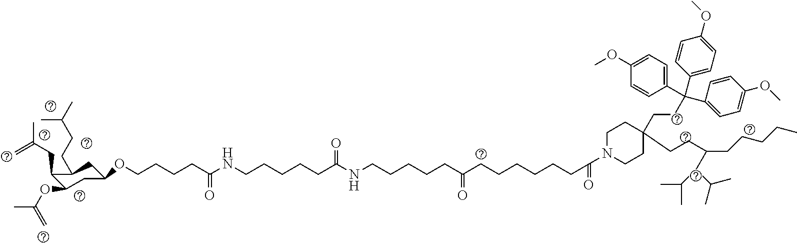
After Attachment to Oligos (Nomenclature) 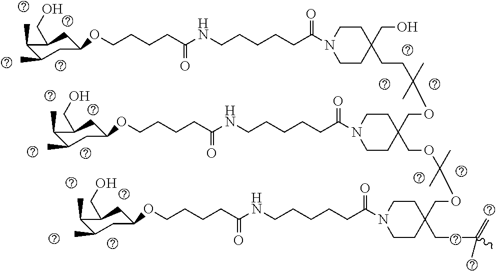
(GalNAc3-(PS)2-p) 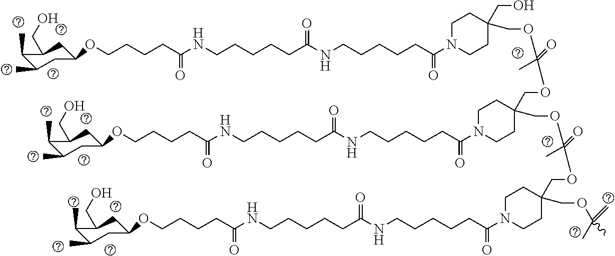
(GalNAc4-(PS)2-p) 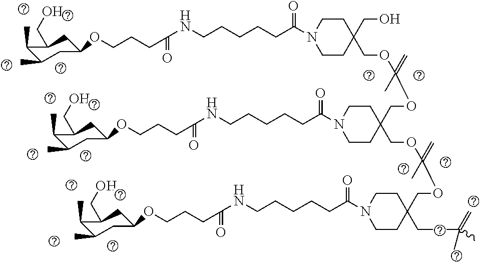
(GalNAc5-(PS)2-p) 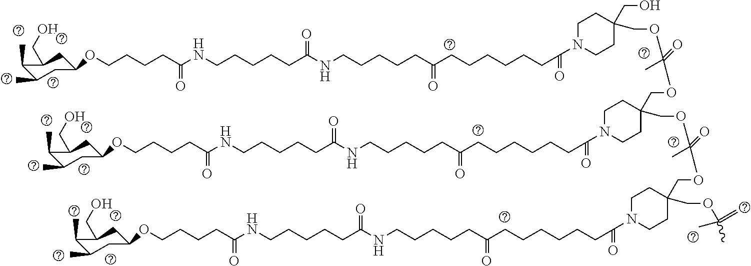
(GalNAc6-(PS)2-p)

In some embodiments, the disclosed ASOs can be conjugated to a monomeric GalNAc or a dimeric GalNAc, such as GalNAc 4, which is show below in two forms (phosphate and phosphorothioate):

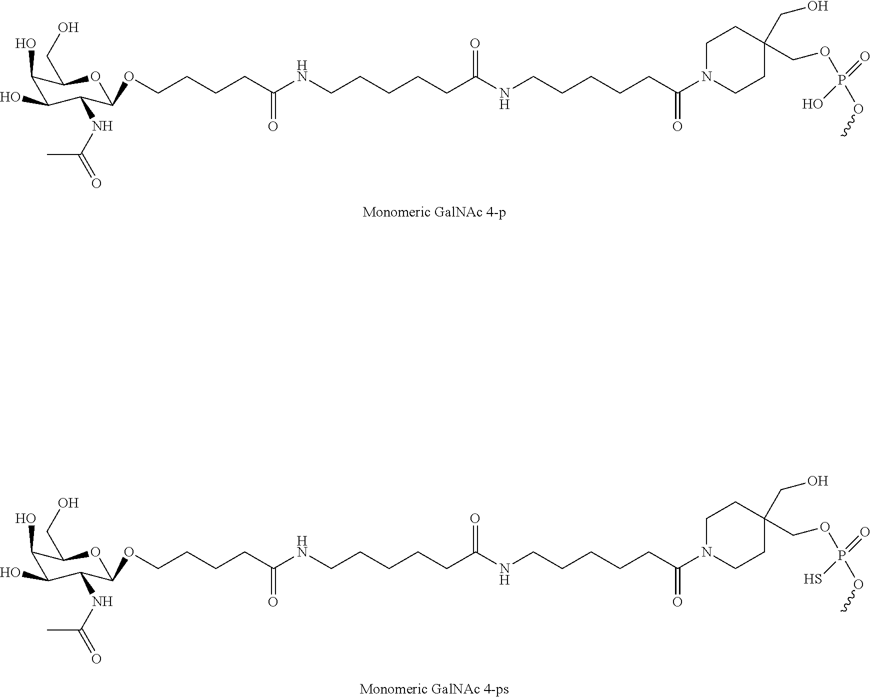

### Exemplary Antisense Oligonucleotides

As described above, the ASOs disclosed herein can comprise a modified nucleotide such as locked nucleoside, 2′ substituted nucleoside, 3′ substituted nucleoside, or a combination thereof. Additionally or alternatively, the ASOs disclosed herein can comprise a modified nucleotide such as (8nh)G, (2s)T, (8nh)A, (5oh)C, or a combination thereof. Additionally or alternatively, the ASOs disclosed herein can comprise abasic monomer. Additionally or alternatively, the ASOs disclosed herein can comprise at least one phosphorothioate internucleoside linkage. Table 1 provides exemplary ASO sequences including nucleotide modifications. Table 2 provides examples of unmodified nucleotide sequences of preferred ASO sequences, shown as the unmodified deoxynucleotide sequence.

TABLE 1

| | SEQ | |
|---|---|---|
| | ID | |
| ASO ID # | NO | ASO Sequence (5′ to 3′) |
| ASO-1 | 2 | moeGpsmoe(5m)CpsmoeApsmoeGpsmoeApsGpsGpsTpsGpsApsApsGps(5m) |
| | | CpsGpsApsmoeApsmoeGpsmoeTpsmoeGpsmoe(5m)C |
| ASO-2 | 3 | moeGpsmoeTpsmoeGpsmoeApsmoeApsGps(5m)CpsGpsApsApsGpsTpsGps |
| | | (5m)CpsAps(moe5m)CpsmoeAps(moe5m)CpsmoeGpsmoeG |
| ASO-3 | 4 | moeGpsmoeGpsmoeTpsmoeGpsmoeApsApsGps(5m)CpsGpsApsApsGpsTps |
| | | Gps(5m)CpsmoeAps(moe5m)CpsmoeAps(moe5m)CpsmoeG |
| ASO-4 | 5 | moeApsmoeGpsmoeGpsmoeTpsmoeGpsApsApsGps(5m)CpsGpsApsApsGps |
| | | TpsGps(moe5m)CpsmoeAps(moe5m)CpsmoeAps(moe5m)C |
| ASO-5 | 6 | moeApsmoeGpsmoeApsmoeGpsmoeGpsTpsGpsApsApsGps(5m)CpsGpsAps |
| | | ApsGpsmoeTpsmoeGps(moe5m)CpsmoeAps(moe5m)C |
| ASO-6 | 7 | moe(5m)CpsmoeApsmoeGpsmoeApsmoeGpsGpsTpsGpsApsApsGps(5m)Cps |
| | | GpsApsApsmoeGpsmoeTpsmoeGpsmoe(5m)CpsmoeA |
| ASO-7 | 8 | moeTpsmoeGps(moe5m)CpsmoeApsmoeGpsApsGpsGpsTpsGpsApsApsGps |
| | | (5m)CpsGpsmoeApsmoeApsmoeGpsmoeTpsmoeG |
| ASO-8 | 9 | (moe5m)CpsmoeGpsmoeTpsmoeGps(moe5m)CpsApsGpsApsGpsGpsTpsGps |
| | | ApsApsGps(moe5m)CpsmoeGpsmoeApsmoeApsmoeG |
| ASO-9 | 10 | moeApsmoe(5m)CpsmoeGpsmoeTpsmoeGps(5m)CpsApsGpsApsGpsGpsTps |
| | | GpsApsApsmoeGpsmoe(5m)CpsmoeGpsmoeApsmoeA |
| ASO-10 | 11 | moeApsmoeApsmoeApsmoe(5m)CpsmoeGps(5m)Cps(5m)CpsGps(5m)Cps |
| | | ApsGpsAps(5m)CpsAps(5m)CpsmoeApsmoeTpsmoe(5m)Cpsmoe(5m) |
| | | CpsmoeA |
| ASO-11 | 12 | moeApsmoeApsmoeApsmoeApsmoe(5m)CpsGps(5m)CpsGps(5m)CpsAps |
| | | GpsAps(5m)CpsApsmoe(5m)CpsmoeApsmoeTpsmoe(5m)Cpsmoe(5m)C |
| ASO-12 | 13 | moeTpsmoeApsmoeApsmoeApsmoeAps(5m)CpsGps(5m)Cps(5m)CpsGps |
| | | (5m)CpsApsGpsAps(5m)CpsmoeApsmoe(5m)CpsmoeApsmoeTpsmoe(5m)c |
| ASO-13 | 14 | moeApsmoeTpsmoeApsmoeApsmoeApsAps(5m)CpsGps(5m)Cps(5m)Cps |
| | | Gps(5m)CpsApsGpsAps(5m)CpsmoeApsmoe(5m)CpsmoeApsmoeTpsmoe |
| | | (5m)C |
| ASO-14 | 15 | moeGpsmoeApsmoeTpsmoeApsmoeApsApsAps(5m)CpsGps(5m)Cps(5m) |
| | | CpsGps(5m)CpsApsGpsApsmoe(5m)CpsmoeApsmoe(5m)CpsmoeApsmoeT |
| ASO-15 | 16 | moeTpsmoeGpsmoeApsmoeTpsmoeApsApsApsAps(5m)CpsGps(5m)Cps |
| | | (5m)CpsGps(5m)CpsApsmoeGpsmoeApsmoe(5m)CsmoeApsmoe(5m)C |
| ASO-16 | 17 | moeApsmoeTpsmoeGpsmoeApsmoeTpsApsApsApsAps(5m)CpsGps(5m)Cps |
| | | (5m)CpsGps(5m)CpsmoeApsmoeGpsmoeApsmoe(5m)CpsmoeA |
| ASO-17 | 18 | mCpsmGpsmUpsmCpsmUpsrCrCrArCrUrUrCrGrCrUpsmUpsmCpsmApsm |
| | | CpsmG |
| ASO-18 | 19 | moeGpsmoe(5m)CpsmoeApsmoeGpsmoeApsmGpsmGpsTpsGpsApsApsGps |
| | | (5m)CpsGpsApsmoeApsmoeGpsmoeTpsmoeGpsmoe(5m)C |
| ASO-19 | 20 | moeGpsmoe(5m)CpsmoeApsmoeGpsmoeApsrGpsrGpsTpsGpsApsApsGps |
| | | (5m)CpsGpsApsmoeApsmoeGpsmoeTpsmoeGpsmoe(5m)C |
| ASO-20 | 21 | rUpsrUrGmoeGpsmoe(5m)CpsmoeApsmoeGpsmoeApsGpsGpsTpsGpsAps |
| | | ApsGps(5m)CpsGpsApsmoeApsmoeGpsmoeTpsmoeGpsmoe(5m)C |
| ASO-21 | 22 | rUrUpsrGmoeGpsmoe(5m)CpsmoeApsmoeGpsmoeApsGpsGpsTpsGpsAps |
| | | ApsGps(5m)CpsGpsApsmoeApsmoeGpsmoeTpsmoeGpsmoe(5m)C |
| ASO-22 | 23 | rUpsrUpsrGmoeGpsmoe(5m)CpsmoeApsmoeGpsmoeApsGpsGpsTpsGpsAps |
| | | ApsGps(5m)CpsGpsApsmoeApsmoeGpsmoeTpsmoeGpsmoe(5m)C |
| ASO-23 | 24 | rUpsrGpsrUmoeGpsmoe(5m)CpsmoeApsmoeGpsmoeApsGpsGpsTpsGpsAps |
| | | ApsGps(5m)CpsGpsApsmoeApsmoeGpsmoeTpsmoeGpsmoe(5m)C |
| ASO-24 | 25 | ocpGpsmoe(5m)CpsmoeApsmoeGpsmoeApsGpsGpsTpsGpsApsApsGps(5m) |
| | | CpsGpsApsmoeApsmoeGpsmoeTpsmoeGpsmoe(5m)C |
| ASO-25 | 26 | moeGpsocpCpsmoeApsmoeGpsmoeApsGpsGpsTpsGpsApsApsGps(5m)Cps |
| | | GpsApsmoeApsmoeGpsmoeTpsmoeGpsmoe(5m)C |
| ASO-26 | 27 | moeGpsmoe(5m)CpsocpApsmoeGpsmoeApsGpsGpsTpsGpsApsApsGps(5m) |
| | | CpsGpsApsmoeApsmoeGpsmoeTpsmoeGpsmoe(5m)C |
| ASO-27 | 28 | moeGpsmoe(5m)CpsmoeApsocpGpsmoeApsGpsGpsTpsGpsApsApsGps(5m) |
| | | CpsGpsApsmoeApsmoeGpsmoeTpsmoeGpsmoe(5m)C |
| ASO-28 | 29 | moeGpsmoe(5m)CpsmoeApsmoeGpsocpApsGpsGpsTpsGpsApsApsGps(5m) |
| | | CpsGpsApsmoeApsmoeGpsmoeTpsmoeGpsmoe(5m)C |
| ASO-29 | 30 | moeGpsmoe(5m)CpsmoeApsmoeGpsmoeApsGpsGpsTpsGpsApsApsGps(5m) |
| | | CpsGpsApsocpApsmoeGpsmoeTpsmoeGpsmoe(5m)C |
| ASO-30 | 31 | moeGpsmoe(5m)CpsmoeApsmoeGpsmoeApsGpsGpsTpsGpsApsApsGps(5m) |
| | | CpsGpsApsmoeApsocpGpsmoeTpsmoeGpsmoe(5m)C |
| ASO-31 | 32 | moeGpsmoe(5m)CpsmoeApsmoeGpsmoeApsGpsGpsTpsGpsApsApsGps(5m) |
| | | CpsGpsApsmoeApsmoeGpsocpUpsmoeGpsmoe(5m)C |
| ASO-32 | 33 | moeGpsmoe(5m)CpsmoeApsmoeGpsmoeApsGpsGpsTpsGpsApsApsGps(5m) |
| | | CpsGpsApsmoeApsmoeGpsmoeTpsocpGpsmoe(5m)C |
| ASO-33 | 34 | moeGpsmoe(5m)CpsmoeApsmoeGpsmoeApsGpsGpsTpsGpsApsApsGps(5m) |
| | | CpsGpsApsmoeApsmoeGpsmoeTpsmoeGpsocpC |
| ASO-34 | 35 | moeGpsmoe(5m)CpsmoeApsmoeGpsmoeApsGpsGpsTpsGpsApsApsGps(5m) |
| | | CpsGpsApsmoeApsmoeGpsmoeUpsmoeGpsmoe(5m)C |
| ASO-35 | 36 | rUmoeGpsmoe(5m)CpsmoeApsmoeGpsmoeApsGpsGpsTpsGpsApsApsGps |
| | | (5m)CpsGpsApsmoeApsmoeGpsmoeTpsmoeGpsmoe(5m)C |
| ASO-36 | 37 | rUpsrUmoeGpsmoe(5m)CpsmoeApsmoeGpsmoeApsGpsGpsTpsGpsApsAps |
| | | Gps(5m)CpsGpsApsmoeApsmoeGpsmoeTpsmoeGpsmoe(5m)C |
| ASO-37 | 38 | rUpsrUpsrUmoeGpsmoe(5m)CpsmoeApsmoeGpsmoeApsGpsGpsTpsGpsAps |
| | | ApsGps(5m)CpsGpsApsmoeApsmoeGpsmoeTpsmoeGpsmoe(5m)C |
| ASO-38 | 39 | rUpsrGmoeGpsmoe(5m)CpsmoeApsmoeGpsmoeApsGpsGpsTpsGpsApsAps |
| | | Gps(5m)CpsGpsApsmoeApsmoeGpsmoeTpsmoeGpsmoe(5m)C |
| ASO-38 | 40 | rUrUrGmoeGpsmoe(5m)CpsmoeApsmoeGpsmoeApsGpsGpsTpsGpsApsAps |
| | | Gps(5m)CpsGpsApsmoeApsmoeGpsmoeTpsmoeGpsmoe(5m)C |
| ASO-40 | 41 | rUrUrGrUmoeGpsmoe(5m)CpsmoeApsmoeGpsmoeApsGpsGpsTpsGpsAps |
| | | ApsGps(5m)CpsGpsApsmoeApsmoeGpsmoeTpsmoeGpsmoe(5m)C |
| ASO-41 | 42 | lmGpsmoe(5m)CpsmoeApsmoeGpsmoeApsGpsGpsTpsGpsApsApsGps(5m) |
| | | CpsGpsApsmoeApsmoeGpsmoeTpsmoeGpsmoe(5m)C |
| ASO-42 | 43 | moeGpsmoe(5m)CpsmoeApslmGpsmoeApsGpsGpsTpsGpsApsApsGps(5m) |
| | | CpsGpsApsmoeApsmoeGpsmoeTpsmoeGpsmoe(5m)C |
| ASO-43 | 44 | moeGpsmoe(5m)CpsmoeApsmoeGpsmoeApsGpsGpsTpsGpsApsApsGps(5m) |
| | | CpsGpsApsmoeApslmGpsmoeTpsmoeGpsmoe(5m)C |
| ASO-44 | 45 | moeGpsmoe(5m)CpsmoeApsmoeGpsmoeApsGpsGpsTpsGpsApsApsGps(5m) |
| | | CpsGpsApsmoeApsmoeGpsmoeTpslmGpsmoe(5m)C |
| ASO-45 | 46 | rUrGrUrUrGrUmoeGpsmoe(5m)CpsmoeApsmoeGpsmoeApsGpsGpsTpsGps |
| | | ApsApsGps(5m)CpsGpsApsmoeApsmoeGpsmoeTpsmoeGpsmoe(5m)C |
| ASO-46 | 47 | rUrUrArUmoeGpsmoe(5m)CpsmoeApsmoeGpsmoeApsGpsGpsTpsGpsAps |
| | | ApsGps(5m)CpsGpsApsmoeApsmoeGpsmoeTpsmoeGpsmoe(5m)C |
| ASO-47 | 48 | rUrArUrUrArUmoeGpsmoe(5m)CpsmoeApsmoeGpsmoeApsGpsGpsTpsGps |
| | | ApsApsGps(5m)CpsGpsApsmoeApsmoeGpsmoeTpsmoeGpsmoe(5m)C |
| ASO-48 | 49 | moeGpsmoe(5m)CpsmoeApsmoeGpsmoeApsocpGpsGpsTpsGpsApsApsGps |
| | | (5m)CpsGpsApsmoeApsmoeGpsmoeTpsmoeGpsmoe(5m)C |
| ASO-49 | 50 | moeGpsmoe(5m)CpsmoeApsmoeGpsmoeApsGpsocpGpsTpsGpsApsApsGps |
| | | (5m)CpsGpsApsmoeApsmoeGpsmoeTpsmoeGpsmoe(5m)C |
| ASO-50 | 51 | moeGpsmoe(5m)CpsmoeApsmoeGpsmoeApsGpsGpsocpUpsGpsApsApsGps |
| | | (5m)CpsGpsApsmoeApsmoeGpsmoeTpsmoeGpsmoe(5m)C |
| ASO-51 | 52 | moeGpsmoe(5m)CpsmoeApsmoeGpsmoeApsomcpGpsGpsTpsGpsApsAps |
| | | Gps(5m)CpsGpsApsmoeApsmoeGpsmoeTpsmoeGpsmoe(5m)C |
| ASO-52 | 53 | moeGpsmoe(5m)CpsmoeApsmoeGpsmoeApsGpsomcpGpsTpsGpsApsAps |
| | | Gps(5m)CpsGpsApsmoeApsmoeGpsmoeTpsmoeGpsmoe(5m)C |
| ASO-53 | 54 | moeGpsmoe(5m)CpsmoeApsmoeGpsmoeApsGpsGpsomcpUpsGpsApsAps |
| | | Gps(5m)CpsGpsApsmoeApsmoeGpsmoeTpsmoeGpsmoe(5m)C |
| ASO-54 | 55 | moeGpsmoe(5m)CpsmoeApsmoeGpsmoeApsGpslmGpsTpsGpsApsApsGps |
| | | (5m)CpsGpsApsmoeApsmoeGpsmoeTpsmoeGpsmoe(5m)C |
| ASO-55 | 56 | moeGpsmoe(5m)CpsmoeApsmoeGpsmoeApslmGpsGpsTpsGpsApsApsGps |
| | | (5m)CpsGpsApsmoeApsmoeGpsmoeTpsmoeGpsmoe(5m)C |
| ASO-56 | 57 | moeGpsmoe(5m)CpsmoeApsmoeGpsmoeApsGpsGpslmUpsGpsApsApsGps |
| | | (5m)CpsGpsApsmoeApsmoeGpsmoeTpsmoeGpsmoe(5m)C |
| ASO-57 | 58 | moeGpsmoe(5m)CpsmoeApsmoeGpsmoeApsGpsGpsTpsGpsApsApsGps |
| | | (5m)CpsGpsApsmoeApsmoeGpsmoeTpsmoeGpsmoe(5m)CrU |
| ASO-58 | 59 | moeGpsmoe(5m)CpsmoeApsmoeGpsmoeApsGpsGpsTpsGpsApsApsGps |
| | | (5m)CpsGpsApsmoeApsmoeGpsmoeTpsmoeGpsmoe(5m)CrUpsrU |
| ASO-59 | 60 | moeGpsmoe(5m)CpsmoeApsmoeGpsmoeApsGpsGpsTpsGpsApsApsGps(5m) |
| | | CpsGpsApsmoeApsmoeGpsmoeTpsmoeGpsmoe(5m)CrUpsrUpsrU |
| ASO-60 | 61 | moeGpsmoe(5m)CpsmoeApsmoeGpsmoeApsGpsGpsTpsGpsApsApsGps(5m) |
| | | CpsGpsApsmoeApsmoeGpsmoeTpsmoeGpsmoe(5m)CrUpsrG |
| ASO-61 | 62 | moeGpsmoe(5m)CpsmoeApsmoeGpsmoeApsGpsGpsTpsGpsApsApsGps(5m) |
| | | CpsGpsApsmoeApsmoeGpsmoeTpsmoeGpsmoe(5m)CpsrUpsrG |
| ASO-62 | 63 | moeGpsmoe(5m)CpsmoeApsmoeGpsmoeApsGpsGpsTpsGpsApsApsGps(5m) |
| | | CpsGpsApsmoeApsmoeGpsmoeTpsmoeGpsmoe(5m)CrUrUrG |
| ASO-63 | 64 | moeGpsmoe(5m)CpsmoeApsmoeGpsmoeApsGpsGpsTpsGpsApsApsGps(5m) |
| | | CpsGpsApsmoeApsmoeGpsmoeTpsmoeGpsmoe(5m)CrUpsrUrG |
| ASO-64 | 65 | moeGpsmoe(5m)CpsmoeApsmoeGpsmoeApsGpsGpsTpsGpsApsApsGps(5m) |
| | | CpsGpsApsmoeApsmoeGpsmoeTpsmoeGpsmoe(5m)CrUpsrUpsrG |
| ASO-65 | 66 | moeGpsmoe(5m)CpsmoeApsmoeGpsmoeApsGpsGpsTpsGpsApsApsGps(5m) |
| | | CpsGpsApsmoeApsmoeGpsmoeTpsmoeGpsmoe(5m)CrUpsrGpsrU |
| ASO-66 | 67 | moeGpsmoe(5m)CpsmoeApsmoeGpsmoeApsGpsGpsTpsGpsApsApsGps(5m) |
| | | CpsGpsApsmoeApsmoeGpsrUpsmoeGpsmoe(5m)C |
| ASO-67 | 68 | omcpGpsmoe(5m)CpsmoeApsmoeGpsmoeApsGpsGpsTpsGpsApsApsGps |
| | | (5m)CpsGpsApsmoeApsmoeGpsmoeTpsmoeGpsmoe(5m)C |
| ASO-68 | 69 | moeGpsomcpCpsmoeApsmoeGpsmoeApsGpsGpsTpsGpsApsApsGps(5m) |
| | | CpsGpsApsmoeApsmoeGpsmoeTpsmoeGpsmoe(5m)C |
| ASO-69 | 70 | moeGpsmoe(5m)CpsomcpApsmoeGpsmoeApsGpsGpsTpsGpsApsApsGps |
| | | (5m)CpsGpsApsmoeApsmoeGpsmoeTpsmoeGpsmoe(5m)C |
| ASO-70 | 71 | moeGpsmoe(5m)CpsmoeApsomcpGpsmoeApsGpsGpsTpsGpsApsApsGps |
| | | (5m)CpsGpsApsmoeApsmoeGpsmoeTpsmoeGpsmoe(5m)C |
| ASO-71 | 72 | moeGpsmoe(5m)CpsmoeApsmoeGpsomcpApsGpsGpsTpsGpsApsApsGps |
| | | (5m)CpsGpsApsmoeApsmoeGpsmoeTpsmoeGpsmoe(5m)C |
| ASO-72 | 73 | moeGpsmoe(5m)CpsmoeApsmoeGpsmoeApsGpsGpsTpsGpsApsApsGps |
| | | (5m)CpsGpsApsomcpApsmoeGpsmoeTpsmoeGpsmoe(5m)C |
| ASO-73 | 74 | moeGpsmoe(5m)CpsmoeApsmoeGpsmoeApsGpsGpsTpsGpsApsApsGps |
| | | (5m)CpsGpsApsmoeApsomcpGpsmoeTpsmoeGpsmoe(5m)C |
| ASO-74 | 75 | moeGpsmoe(5m)CpsmoeApsmoeGpsmoeApsGpsGpsTpsGpsApsApsGps |
| | | (5m)CpsGpsApsmoeApsmoeGpsomcpUpsmoeGpsmoe(5m)C |
| ASO-75 | 76 | moeGpsmoe(5m)CpsmoeApsmoeGpsmoeApsGpsGpsTpsGpsApsApsGps |
| | | (5m)CpsGpsApsmoeApsmoeGpsmoeTpsomcpGpsmoe(5m)C |
| ASO-76 | 77 | moeGpsmoe(5m)CpsmoeApsmoeGpsmoeApsGpsGpsTpsGpsApsApsGps |
| | | (5m)CpsGpsApsmoeApsmoeGpsmoeTpsmoeGpsomcpC |
| ASO-77 | 78 | mGpsmoe(5m)CpsmoeApsmoeGpsmoeApsGpsGpsTpsGpsApsApsGps(5m) |
| | | CpsGpsApsmoeApsmoeGpsmoeTpsmoeGpsmoe(5m)C |
| ASO-78 | 79 | moeGpsm(5m)CpsmoeApsmoeGpsmoeApsGpsGpsTpsGpsApsApsGps(5m) |
| | | CpsGpsApsmoeApsmoeGpsmoeTpsmoeGpsmoe(5m)C |
| ASO-79 | 80 | moeGpsmoe(5m)CpsmApsmoeGpsmoeApsGpsGpsTpsGpsApsApsGps(5m) |
| | | CpsGpsApsmoeApsmoeGpsmoeTpsmoeGpsmoe(5m)C |
| ASO-80 | 81 | moeGpsmoe(5m)CpsmoeApsmGpsmoeApsGpsGpsTpsGpsApsApsGps(5m) |
| | | CpsGpsApsmoeApsmoeGpsmoeTpsmoeGpsmoe(5m)C |
| ASO-81 | 82 | moeGpsmoe(5m)CpsmoeApsmoeGpsmApsGpsGpsTpsGpsApsApsGps(5m) |
| | | CpsGpsApsmoeApsmoeGpsmoeTpsmoeGpsmoe(5m)C |
| ASO-82 | 83 | moeGpsmoe(5m)CpsmoeApsmoeGpsmoeApsGpsGpsTpsGpsApsApsGps |
| | | (5m)CpsGpsApsmApsmoeGpsmoeTpsmoeGpsmoe(5m)C |
| ASO-83 | 84 | moeGpsmoe(5m)CpsmoeApsmoeGpsmoeApsGpsGpsTpsGpsApsApsGps |
| | | (5m)CpsGpsApsmoeApsmGpsmoeTpsmoeGpsmoe(5m)C |
| ASO-84 | 85 | moeGpsmoe(5m)CpsmoeApsmoeGpsmoeApsGpsGpsTpsGpsApsApsGps |
| | | (5m)CpsGpsApsmoeApsmoeGpsmUpsmoeGpsmoe(5m)C |
| ASO-85 | 86 | moeGpsmoe(5m)CpsmoeApsmoeGpsmoeApsGpsGpsTpsGpsApsApsGps |
| | | (5m)CpsGpsApsmoeApsmoeGpsmoeTpsmGpsmoe(5m)C |
| ASO-86 | 87 | moeGpsmoe(5m)CpsmoeApsmoeGpsmoeApsGpsGpsTpsGpsApsApsGps |
| | | (5m)CpsGpsApsmoeApsmoeGpsmoeTpsmoeGpsm(5m)C |
| ASO-87 | 88 | moeGpsmoe(5m)CpsmoeDAPpsmoeGpsmoeApsGpsGpsTpsGpsApsApsGps |
| | | (5m)CpsGpsApsmoeApsmoeGpsmoeTpsmoeGpsmoe(5m)C |
| ASO-88 | 89 | moeGpsmoe(5m)CpsmoeApsmoeGpsmoeDAPpsGpsGpsTpsGpsApsApsGps |
| | | (5m)CpsGpsApsmoeApsmoeGpsmoeTpsmoeGpsmoe(5m)C |
| ASO-89 | 90 | moeGpsmoe(5m)CpsmoeApsmoeGpsmoeApsGpsGpsTpsGpsApsApsGps(5m) |
| | | CpsGpsApsmoeDAPpsmoeGpsmoeTpsmoeGpsmoe(5m)C |
| ASO-90 | 91 | mGpsmoe(5m)CpsmoeApsmGpsmoeApsGpsGpsTpsGpsApsApsGps(5m)Cps |
| | | GpsApsmoeApsmoeGpsmoeTpsmoeGpsmoe(5m)C |
| ASO-91 | 92 | moeGpsmoe(5m)CpsmApsmoeGpsmApsGpsGpsTpsGpsApsApsGps(5m)Cps |
| | | GpsApsmoeApsmoeGpsmoeTpsmoeGpsmoe(5m)C |
| ASO-92 | 93 | moeGpsmoe(5m)CpsmoeApsmoeGpsmoeApsGpsGpsTpsGpsApsApsGps(5m) |
| | | CpsGpsApsmoeApsmGpsmoeTpsmGpsmoe(5m)C |
| ASO-93 | 94 | mGpsmoe(5m)CpsmApsmoeGpsmApsGpsGpsTpsGpsApsApsGps(5m)Cps |
| | | GpsApsmoeApsmoeGpsmoeTpsmoeGpsmoe(5m)C |
| ASO-94 | 95 | moeGpsmoe(5m)CpsmoeApsmoeGpsmoeApsGpsGpsTpsGpsApsApsGps(5m) |
| | | CpsGpsApsmApsmGpsmoeTpsmGpsmoe(5m)C |
| ASO-95 | 96 | mGpsmoe(5m)CpsmoeApsmoeGpsmoeApsGpsGpsTpsGpsApsApsGps(5m) |
| | | CpsGpsApsmoeApsmoeGpsmoeTpsmoeGpsm(5m)C |
| ASO-96 | 97 | moeGpsmoe(5m)CpsmoeApsmoeGpsmoeApsGpsGpsTpsGpsApss?ApsGps |
| | | (5m)CpsGpsApsmoeApsmoeGpsmoeTpsmoeGpsmoe(5m)C |
| ASO-97 | 98 | moeGpsmoe(5m)CpsmoeApsmoeGpsmoeApsGpsGpsTpsGpsApsApss?Gps |
| | | (5m)CpsGpsApsmoeApsmoeGpsmoeTpsmoeGpsmoe(5m)C |
| ASO-98 | 99 | moeGpsmoe(5m)CpsmoeApsmoeGpsmoeApsGpsGpsTpsGpsApsApsGps(5m) |
| | | CpsGpsApss?moeApsmoeGpsmoeTpsmoeGpsmoe(5m)C |
| ASO-99 | 100 | moeGpsmoe(5m)CpsmoeApsmoeGpsmoeAps(8nh)GpsGpsTpsGpsApsAps |
| | | Gps(5m)CpsGpsApsmoeApsmoeGpsmoeTpsmoeGpsmoe(5m)C |
| ASO-100 | 101 | moeGpsmoe(5m)CpsmoeApsmoeGpsmoeApsGps(8nh)GpsTpsGpsApsAps |
| | | Gps(5m)CpsGpsApsmoeApsmoeGpsmoeTpsmoeGpsmoe(5m)C |
| ASO-101 | 102 | moeGpsmoe(5m)CpsmoeApsmoeGpsmoeApsGpsGps(2s)TpsGpsApsApsGps |
| | | (5m)CpsGpsApsmoeApsmoeGpsmoeTpsmoeGpsmoe(5m)C |
| ASO-102 | 103 | moeGpsmoe(5m)CpsmoeApsmoeGpsmoeApsGpss?GpsTpsGpsApsApsGps |
| | | (5m)CpsGpsApsmoeApsmoeGpsmoeTpsmoeGpsmoe(5m)C |
| ASO-103 | 104 | moeGpsmoe(5m)CpsmoeApsmoeGpsmoeApsGpsGpss?TpsGpsApsApsGps |
| | | (5m)CpsGpsApsmoeApsmoeGpsmoeTpsmoeGpsmoe(5m)C |
| ASO-104 | 105 | moeGpsmoe(5m)CpsmoeApsmoeGpsmoeApsGpsGpsTpss?GpsApsApsGps |
| | | (5m)CpsGpsApsmoeApsmoeGpsmoeTpsmoeGpsmoe(5m)C |
| ASO-105 | 106 | moeGpsmoe(5m)CpsmoeApsmoeGpsmoeApsGpsGpsTpsGpss?ApsApsGps |
| | | (5m)CpsGpsApsmoeApsmoeGpsmoeTpsmoeGpsmoe(5m)C |
| ASO-106 | 107 | moeGpsmoe(5m)CpsmoeApsmoeGpsmoeApsGpsGpsTpsGpsApsApsGpss? |
| | | (5m)CpsGpsApsmoeApsmoeGpsmoeTpsmoeGpsmoe(5m)C |
| ASO-107 | 108 | moeGpsmoe(5m)CpsmoeApsmoeGpsmoeApsGpsGpsTpsGpsApsApsGps(5m) |
| | | Cpss?GpsApsmoeApsmoeGpsmoeTpsmoeGpsmoe(5m)C |
| ASO-108 | 109 | moeGpsmoe(5m)CpsmoeApsmoeGpsmoeApsGpsGpsTpsGpsApsApsGps(5m) |
| | | CpsGpss?ApsmoeApsmoeGpsmoeTpsmoeGpsmoe(5m)C |
| ASO-109 | 110 | moe(5m)CpsmoeApsmoeGpsmoeApsmoeGps(8nh)GpsTpsGpsApsApsGps |
| | | (5m)CpsGpsApsApsmoeGpsmoeTpsmoeGpsmoe(5m)CpsmoeA |
| ASO-110 | 111 | moe(5m)CpsmoeApsmoeGpsmoeApsmoeGpsGps(2s)TpsGpsApsApsGps(5m) |
| | | CpsGpsApsApsmoeGpsmoeTpsmoeGpsmoe(5m)CpsmoeA |
| ASO-111 | 112 | moe(5m)CpsmoeApsmoeGpsmoeApsmoeGpsGpsTps(8nh)GpsApsApsGps |
| | | (5m)CpsGpsApsApsmoeGpsmoeTpsmoeGpsmoe(5m)CpsmoeA |
| ASO-112 | 113 | moe(5m)CpsmoeApsmoeGpsmoeApsmoeGpsGpsTpsGps(8nh)ApsApsGps |
| | | (5m)CpsGpsApsApsmoeGpsmoeTpsmoeGpsmoe(5m)CpsmoeA |
| ASO-113 | 114 | moe(5m)CpsmoeApsmoeGpsmoeApsmoeGpsGpsTpsGpsAps(8nh)ApsGps |
| | | (5m)CpsGpsApsApsmoeGpsmoeTpsmoeGpsmoe(5m)CpsmoeA |
| ASO-114 | 115 | moe(5m)CpsmoeApsmoeGpsmoeApsmoeGpsGpsTpsGpsApsAps(8nh)Gps |
| | | (5m)CpsGpsApsApsmoeGpsmoeTpsmoeGpsmoe(5m)CpsmoeA |
| ASO-115 | 116 | moe(5m)CpsmoeApsmoeGpsmoeApsmoeGpsGpsTpsGpsApsApsGps(5oh) |
| | | CpsGpsApsApsmoeGpsmoeTpsmoeGpsmoe(5m)CpsmoeA |
| ASO-116 | 117 | moe(5m)CpsmoeApsmoeGpsmoeApsmoeGpsGpsTpsGpsApsApsGps(5m) |
| | | Cps(8nh)GpsApsApsmoeGpsmoeTpsmoeGpsmoe(5m)CpsmoeA |
| ASO-117 | 118 | moe(5m)CpsmoeApsmoeGpsmoeApsmoeGpsGpsTpsGpsApsApsGps(5m) |
| | | CpsGps(8nh)ApsApsmoeGpsmoeTpsmoeGpsmoe(5m)CpsmoeA |
| ASO-118 | 119 | moe(5m)CpsmoeApsmoeGpsmoeApsmoeGpsGpsTpsGpsApsApsGps(5m) |
| | | CpsGpsAps(8nh)ApsmoeGpsmoeTpsmoeGpsmoe(5m)CpsmoeA |
| ASO-119 | 120 | moe(5m)CpsmoeApsmoeGpsmoeApsGpsGpsTpsGpsApsApsGps(5m)Cps |
| | | GpsApsmoeApsmoeGpsmoeTpsmoeGpsmoe(5m)C |
| ASO-120 | 121 | moeGpsmoe(5m)CpsmoeApsmoeGpsmoeApsGpsGpsTpsGpsApsApsGps(5m) |
| | | CpsGpsApsmoeApsmoeGpsmoeTpsmoeG |
| ASO-121 | 122 | rGpsmoe(5m)CpsmoeApsmoeGpsmoeApsGpsGpsTpsGpsApsApsGps(5m) |
| | | CpsGpsApsmoeApsmoeGpsmoeTpsmoeGpsmoe(5m)C |
| ASO-122 | 123 | moeGpsmoe(5m)CpsmoeApsrGpsmoeApsGpsGpsTpsGpsApsApsGps(5m) |
| | | CpsGpsApsmoeApsmoeGpsmoeTpsmoeGpsmoe(5m)C |
| ASO-123 | 124 | moeGpsmoe(5m)CpsmoeApsmoeGpsrApsGpsGpsTpsGpsApsApsGps(5m) |
| | | CpsGpsApsmoeApsmoeGpsmoeTpsmoeGpsmoe(5m)C |
| ASO-124 | 125 | moeGpsmoe(5m)CpsmoeApsmoeGpsmoeApsGpsGpsTpsGpsApsApsGps(5m) |
| | | CpsGpsApsmoeApsrGpsmoeTpsmoeGpsmoe(5m)C |
| ASO-125 | 126 | moeGpsmoe(5m)CpsmoeApsmoeGpsmoeApsGpsGpsTpsGpsApsApsGps(5m) |
| | | CpsGpsApsmoeApsmoeGpsmoeTpsrGpsmoe(5m)C |
| ASO-126 | 127 | moeGpsmoe(5m)CpsmoeApsmoeGpsmoeApsGpsGpsTpsGpsApsApsGps(5m) |
| | | CpsGpsApsrApsmoeGpsmoeTpsmoeGpsmoe(5m)C |
| ASO-127 | 128 | moeGpsmoe(5m)CpsrApsmoeGpsmoeApsGpsGpsTpsGpsApsApsGps(5m) |
| | | CpsGpsApsmoeApsmoeGpsmoeTpsmoeGpsmoe(5m)C |
| ASO-128 | 129 | moeApsmoe(5m)CpsmoeGpsmoeTpsmoeGps(5oh)CpsApsGpsApsGpsGps |
| | | TpsGpsApsApsmoeGpsmoe(5m)CpsmoeGpsmoeApsmoeA |
| ASO-129 | 130 | moeApsmoe(5m)CpsmoeGpsmoeTpsmoeGps(5m)Cps(8nh)ApsGpsApsGps |
| | | GpsTpsGpsApsApsmoeGpsmoe(5m)CpsmoeGpsmoeApsmoeA |
| ASO-130 | 131 | moeApsmoe(5m)CpsmoeGpsmoeTpsmoeGps(5m)CpsAps(8nh)GpsApsGps |
| | | GpsTpsGpsApsApsmoeGpsmoe(5m)CpsmoeGpsmoeApsmoeA |
| ASO-131 | 132 | moeApsmoe(5m)CpsmoeGpsmoeTpsmoeGps(5m)CpsApsGps(8nh)ApsGps |
| | | GpsTpsGpsApsApsmoeGpsmoe(5m)CpsmoeGpsmoeApsmoeA |
| ASO-132 | 133 | moeApsmoe(5m)CpsmoeGpsmoeTpsmoeGps(5m)CpsApsGpsAps(8nh)Gps |
| | | GpsTpsGpsApsApsmoeGpsmoe(5m)CpsmoeGpsmoeApsmoeA |
| ASO-133 | 134 | moeApsmoe(5m)CpsmoeGpsmoeTpsmoeGps(5m)CpsApsGpsApsGps(8nh) |
| | | GpsTpsGpsApsApsmoeGpsmoe(5m)CpsmoeGpsmoeApsmoeA |
| ASO-134 | 135 | moeApsmoe(5m)CpsmoeGpsmoeTpsmoeGps(5m)CpsApsGpsApsGpsGps |
| | | (2s)TpsGpsApsApsmoeGpsmoe(5m)CpsmoeGpsmoeApsmoeA |
| ASO-135 | 136 | moeApsmoe(5m)CpsmoeGpsmoeTpsmoeGps(5m)CpsApsGpsApsGpsGps |
| | | Tps(8nh)GpsApsApsmoeGpsmoe(5m)CpsmoeGpsmoeApsmoeA |
| ASO-136 | 137 | moeApsmoe(5m)CpsmoeGpsmoeTpsmoeGps(5m)CpsApsGpsApsGpsGpsTps |
| | | Gps(8nh)ApsApsmoeGpsmoe(5m)CpsmoeGpsmoeApsmoeA |
| ASO-137 | 138 | moeApsmoe(5m)CpsmoeGpsmoeTpsmoeGps(5m)CpsApsGpsApsGpsGpsTps |
| | | GpsAps(8nh)ApsmoeGpsmoe(5m)CpsmoeGpsmoeApsmoeA |
| ASO-138 | 139 | moeGpsmoe(5m)CpsmoeApsmoeGpsmoeApsGpsGpsTpsGpsApsApsGps |
| | | (5m)CpsGpsApsmoeApsmoeGpsrUpsrGpsmoe(5m)C |
| ASO-139 | 140 | moeGpsmoe(5m)CpsmoeApsmoeGpsApsGpsGpsTpsGpsmoeApsApsGps |
| | | (5m)CpsGpsApsmoeApsInGpsmoeTpslnGpsln(5m)C |
| ASO-140 | 141 | moe(5m)CpsmoeApsmoeGpsmApsmoeGpsGpsTpsGpsApsApsGps(5m)Cps |
| | | GpsApsApsmoeGpsmoeTpsmoeGpsmoe(5m)CpsmoeA |
| ASO-141 | 142 | moe(5m)CpsmoeApsmoeGpsmoeApsmoeGpsmGpsTpsGpsApsApsGps(5m) |
| | | CpsGpsApsApsmoeGpsmoeTpsmoeGpsmoe(5m)CpsmoeA |
| ASO-142 | 143 | moe(5m)CpsmoeApsmoeGpsmApsmGpsGpsTpsGpsApsApsGps(5m)CpsGps |
| | | ApsApsmoeGpsmoeTpsmoeGpsmoe(5m)CpsmoeA |
| ASO-143 | 144 | moe(5m)CpsmoeApsmoeGpsmoeApsmoeGpsGpsTpsGpsApsApsGps(5m)Cps |
| | | GpsApsApsmoeGpsmoeTpsmoeGpsmoe(5m)CpsmoeA |
| ASO-144 | 145 | moe(5m)CpsmoeApsmGpsmoeApsmGpsGpsTpsGpsApsApsGps(5m)CpsGps |
| | | ApsApsmoeGpsmoeTpsmoeGpsmoe(5m)CpsmoeA |
| ASO-145 | 146 | moe(5m)CpsmoeApsmoeGpsmoeApsmoeGpsGpsTpsGpsApsApsGps(5m)Cps |
| | | GpsApsApsmGpsmoeTpsmGpsmoe(5m)CpsmoeA |
| ASO-146 | 147 | moe(5m)CpsmoeApsmGpsmoeApsmGpsGpsTpsGpsApsApsGps(5m)CpsGps |
| | | ApsApsmGpsmoeTpsmoeGpsmoe(5m)CpsmoeA |
| ASO-147 | 148 | moe(5m)CpsmoeApsmGpsmoeApsmGpsGpsTpsGpsApsApsGps(5m)CpsGps |
| | | ApsApsmoeGpsmoeTpsmGpsmoe(5m)CpsmoeA |
| ASO-148 | 149 | moe(5m)CpsmoeApsmGpsmoeApsmoeGpsGpsTpsGpsApsApsGps(5m)Cps |
| | | GpsApsApsmGpsmoeTpsmGpsmoe(5m)CpsmoeA |
| ASO-149 | 150 | moe(5m)CpsmoeApsmoeGpsmoeApsmGpsGpsTpsGpsApsApsGps(5m)Cps |
| | | GpsApsApsmGpsmoeTpsmGpsmoe(5m)CpsmoeA |
| ASO-150 | 151 | moe(5m)CpsmoeApsmGpsmoeApsmGpsGpsTpsGpsApsApsGps(5m)CpsGps |
| | | ApsApsmGpsmoeTpsmGpsmoe(5m)CpsmoeA |
| ASO-151 | 152 | moe(5m)CpsmoeApsmoeGpsmoeApsmoeGpsGpsTpsGpsApsApsGps(5m)Cps |
| | | GpsApsApsmoeGpsmoeTpsmGpsm(5m)CpsmoeA |
| ASO-152 | 153 | moe(5m)CpsmoeApsmoeGpsmoeApsmoeGpsGpsTpsGpsApsApsGps(5m)Cps |
| | | GpsApsApsmoeGpsmoeTpsmGpsm(5m)CpsmA |
| ASO-153 | 154 | moe(5m)CpsmoeApsmoeGpsmoeApsmoeGpsGpsTpsGpsApsApsGps(5m)Cps |
| | | GpsApsApsmGpsmoeTpsmGpsmoe(5m)CpsmA |
| ASO-154 | 155 | moeCpsmoeApsmoeGpsmoeApsmoeGpsGpss?TpsGpsApsApsGpsCpsGps |
| | | ApsApsmoeGpsmoeTpsmoeGpsmoeCpsmoeA |
| ASO-155 | 156 | moeCpsmoeApsmoeGpsmoeApsmoeGpsGpsTpss?GpsApsApsGpsCpsGps |
| | | ApsApsmoeGpsmoeTpsmoeGpsmoeCpsmoeA |
| ASO-156 | 157 | moeCpsmoeApsmoeGpsmoeApsmoeGpsGpsTpsGpss?ApsApsGpsCpsGps |
| | | ApsApsmoeGpsmoeTpsmoeGpsmoeCpsmoeA |
| ASO-157 | 158 | moeCpsmoeApsmoeGpsmoeApsmoeGpsGpsTpsGpsApss?ApsGpsCpsGps |
| | | ApsApsmoeGpsmoeTpsmoeGpsmoeCpsmoeA |
| ASO-158 | 159 | moeCpsmoeApsmoeGpsmoeApsmoeGpsGpsTpsGpsApsApss?GpsCpsGps |
| | | ApsApsmoeGpsmoeTpsmoeGpsmoeCpsmoeA |
| ASO-159 | 160 | moeCpsmoeApsmoeGpsmoeApsmoeGpsGpsTpsGpsApsApsGpss?CpsGps |
| | | ApsApsmoeGpsmoeTpsmoeGpsmoeCpsmoeA |
| ASO-160 | 161 | moeCpsmoeApsmoeGpsmoeApsmoeGpsGpsTpsGpsApsApsGpsCpss?Gps |
| | | ApsApsmoeGpsmoeTpsmoeGpsmoeCpsmoeA |
| ASO-161 | 162 | moeCpsmoeApsmoeGpsmoeApsmoeGpsGpsTpsGpsApsApsGpsCps |
| | | Gpss?ApsApsmoeGpsmoeTpsmoeGpsmoeCpsmoeA |
| ASO-162 | 163 | moeCpsmoeApsmoeGpsmoeApsmoeGpsGpsTpsGpsApsApsGpsCpsGps |
| | | Apss?ApsmoeGpsmoeTpsmoeGpsmoeCpsmoeA |
| ASO-163 | 164 | moeCpsmoeApsmoeGpsmoeApsmoeGpsGpsTpsGpsApsApsGpsCpsGpsAps |
| | | Apss?moeGpsmoeTpsmoeGpsmoeCpsmoeA |
| ASO-164 | 165 | lnCpsmoeApsmoeGpsmoeApsmoeGpsGpsTpsGpsApsApsGpsCpsGpsAps |
| | | ApsmoeGpsmoeTpsmoeGpsmoeCpsmoeA |
| ASO-165 | 166 | moeCpslnApsmoeGpsmoeApsmoeGpsGpsTpsGpsApsApsGpsCpsGpsAps |
| | | ApsmoeGpsmoeTpsmoeGpsmoeCpsmoeA |
| ASO-166 | 167 | moeCpsmoeApslnGpsmoeApsmoeGpsGpsTpsGpsApsApsGpsCpsGpsAps |
| | | ApsmoeGpsmoeTpsmoeGpsmoeCpsmoeA |
| ASO-167 | 168 | moeCpsmoeApsmoeGpslnApsmoeGpsGpsTpsGpsApsApsGpsCpsGpsAps |
| | | ApsmoeGpsmoeTpsmoeGpsmoeCpsmoeA |
| ASO-168 | 169 | moeCpsmoeApsmoeGpsmoeApslnGpsGpsTpsGpsApsApsGpsCpsGpsAps |
| | | ApsmoeGpsmoeTpsmoeGpsmoeCpsmoeA |
| ASO-169 | 170 | moeCpsmoeApsmoeGpsmoeApsmoeGpsGpsTpsGpsApsApsGpsCpsGpsAps |
| | | ApslnGpsmoeTpsmoeGpsmoeCpsmoeA |
| ASO-170 | 171 | moeCpsmoeApsmoeGpsmoeApsmoeGpsGpsTpsGpsApsApsGpsCpsGpsAps |
| | | ApsmoeGpslnTpsmoeGpsmoeCpsmoeA |
| ASO-171 | 172 | moeCpsmoeApsmoeGpsmoeApsmoeGpsGpsTpsGpsApsApsGpsCpsGpsAps |
| | | ApsmoeGpsmoeTpslnGpsmoeCpsmoeA |
| ASO-172 | 173 | moeCpsmoeApsmoeGpsmoeApsmoeGpsGpsTpsGpsApsApsGpsCpsGpsAps |
| | | ApsmoeGpsmoeTpsmoeGpslnCpsmoeA |
| ASO-173 | 174 | moeCpsmoeApsmoeGpsmoeApsmoeGpsGpsTpsGpsApsApsGpsCpsGpsAps |
| | | ApsmoeGpsmoeTpsmoeGpsmoeCpslnA |
| ASO-174 | 175 | ln(5m)CpsmoeApsmoeGpsmoeApsmoeGpsGpsTpsGpsApsApsGps(5m)Cps |
| | | GpsApsApsmoeGpslnTpsmoeGpsmoe(5m)CpsmoeA |
| ASO-175 | 176 | ln(5m)CpsmoeApsmoeGpsmoeApsmoeGpsGpsTpsGpsApsApsGps(5m)Cps |
| | | GpsApsApsmoeGpsmoeTpsmoeGpsln(5m)CpsmoeA |
| ASO-176 | 177 | moe(5m)CpslnApsmoeGpslnApsmoeGpsGpsTpsGpsApsApsGps(5m)CpsGps |
| | | ApsApsmoeGpsmoeTpsmoeGpsmoe(5m)CpsmoeA |
| ASO-177 | 178 | moe(5m)CpslnApsmoeGpsmoeApsmoeGpsGpsTpsGpsApsApsGps(5m)Cps |
| | | GpsApsApsmoeGpsmoeTpsmoeGpsmoe(5m)CpslnA |
| ASO-178 | 179 | moe(5m)CpsmoeApsmoeGpslnApsmoeGpsGpsTpsGpsApsApsGps(5m)Cps |
| | | GpsApsApsmoeGpsmoeTpsmoeGpsmoe(5m)CpslnA |
| ASO-179 | 180 | moe(5m)CpsmoeApslnGpsmoeApslnGpsGpsTpsGpsApsApsGps(5m)CpsGps |
| | | ApsApsmoeGpsmoeTpsmoeGpsmoe(5m)CpsmoeA |
| ASO-180 | 181 | moe(5m)CpsmoeApsmoeGpsmoeApsmoeGpsGpsTpsGpsApsApsGps(5m)Cps |
| | | GpsApsApslnGpsmoeTpslnGpsmoe(5m)CpsmoeA |
| ASO-181 | 182 | moe(5m)CpsmoeApslnGpsmoeApsmoeGpsGpsTpsGpsApsApsGps(5m)Cps |
| | | GpsApsApslnGpsmoeTpsmoeGpsmoe(5m)CpsmoeA |
| ASO-182 | 183 | moe(5m)CpsmoeApslnGpsmoeApsmoeGpsGpsTpsGpsApsApsGps(5m)Cps |
| | | GpsApsApsmoeGpsmoeTpslnGpsmoe(5m)CpsmoeA |
| ASO-183 | 184 | moe(5m)CpsmoeApsmoeGpsmoeApslnGpsGpsTpsGpsApsApsGps(5m)Cps |
| | | GpsApsApsmoeGpsmoeTpslnGpsmoe(5m)CpsmoeA |
| ASO-184 | 185 | moe(5m)CpsmoeApsmoeGpsmoeApslnGpsGpsTpsGpsApsApsGps(5m)Cps |
| | | GpsApsApslnGpsmoeTpsmoeGpsmoe(5m)CpsmoeA |
| ASO-185 | 186 | moe(5m)CpsmoeApsmoeGpsmApsmoeGpsGpsTpsGpsApsAps(8nh)Gps(5m) |
| | | CpsGpsApsApsmoeGpsmoeTpsmoeGpsmoe(5m)CpsmoeA |
| ASO-186 | 187 | moe(5m)CpsmoeApsmoeGpsmoeApsmoeGpsmGpsTpsGpsApsAps(8nh)Gps |
| | | (5m)CpsGpsApsApsmoeGpsmoeTpsmoeGpsmoe(5m)CpsmoeA |
| ASO-187 | 188 | moe(5m)CpsmoeApsmoeGpsmApsmGpsGpsTpsGpsApsAps(8nh)Gps(5m) |
| | | CpsGpsApsApsmoeGpsmoeTpsmoeGpsmoe(5m)CpsmoeA |
| ASO-188 | 189 | moe(5m)CpsmoeApsmoeGpsmApsmGpsmGpsTpsGpsApsAps(8nh)Gps(5m) |
| | | CpsGpsApsApsmoeGpsmoeTpsmoeGpsmoe(5m)CpsmoeA |
| ASO-189 | 190 | moe(5m)CpsmoeApsmGpsmoeApsmGpsGpsTpsGpsApsAps(8nh)Gps(5m) |
| | | CpsGpsApsApsmoeGpsmoeTpsmoeGpsmoe(5m)CpsmoeA |
| ASO-190 | 191 | moe(5m)CpsmoeApsmoeGpsmoeApsmoeGpsGpsTpsGpsApsAps(8nh)Gps |
| | | (5m)CpsGpsApsApsmGpsmoeTpsmGpsmoe(5m)CpsmoeA |
| ASO-191 | 192 | moe(5m)CpsmoeApsmGpsmoeApsmGpsGpsTpsGpsApsAps(8nh)Gps(5m) |
| | | CpsGpsApsApsmGpsmoeTpsmoeGpsmoe(5m)CpsmoeA |
| ASO-192 | 193 | moe(5m)CpsmoeApsmGpsmoeApsmGpsGpsTpsGpsApsAps(8nh)Gps(5m) |
| | | CpsGpsApsApsmoeGpsmoeTpsmGpsmoe(5m)CpsmoeA |
| ASO-193 | 194 | moe(5m)CpsmoeApsmGpsmoeApsmoeGpsGpsTpsGpsApsAps(8nh)Gps(5m) |
| | | CpsGpsApsApsmGpsmoeTpsmGpsmoe(5m)CpsmoeA |
| ASO-194 | 195 | moe(5m)CpsmoeApsmoeGpsmoeApsmGpsGpsTpsGpsApsAps(8nh)Gps(5m) |
| | | CpsGpsApsApsmGpsmoeTpsmGpsmoe(5m)CpsmoeA |
| ASO-195 | 196 | moe(5m)CpsmoeApsmGpsmoeApsmGpsGpsTpsGpsApsAps(8nh)Gps(5m) |
| | | CpsGpsApsApsmGpsmoeTpsmGpsmoe(5m)CpsmoeA |
| ASO-196 | 197 | moe(5m)CpsmoeApsmoeGpsmoeApsmoeGpsGpsTpsGpsApsAps(8nh)Gps |
| | | (5m)CpsGpsApsApsmoeGpsmoeTpsmGpsm(5m)CpsmoeA |
| ASO-197 | 198 | moe(5m)CpsmoeApsmoeGpsmoeApsmoeGpsGpsTpsGpsApsAps(8nh)Gps |
| | | (5m)CpsGpsApsApsmoeGpsmoeTpsmGpsm(5m)CpsmA |
| ASO-198 | 199 | moe(5m)CpsmoeApsmoeGpsmoeApsmoeGpsGpsTpsGpsApsAps(8nh)Gps |
| | | (5m)CpsGpsApsApsmGpsmoeTpsmGpsmoe(5m)CpsmA |
| ASO-199 | 200 | ln(5m)CpsmoeApsmoeGpsmoeApsmoeGpsGpsTpsGpsApsAps(8nh)Gps(5m) |
| | | CpsGpsApsApsmoeGpsmoeTpsmoeGpsmoe(5m)CpsmoeA |
| ASO-200 | 201 | moe(5m)CpslnApsmoeGpsmoeApsmoeGpsGpsTpsGpsApsAps(8nh)Gps(5m) |
| | | CpsGpsApsApsmoeGpsmoeTpsmoeGpsmoe(5m)CpsmoeA |
| ASO-201 | 202 | moe(5m)CpsmoeApslnGpsmoeApsmoeGpsGpsTpsGpsApsAps(8nh)Gps(5m) |
| | | CpsGpsApsApsmoeGpsmoeTpsmoeGpsmoe(5m)CpsmoeA |
| ASO-202 | 203 | moe(5m)CpsmoeApsmoeGpslnApsmoeGpsGpsTpsGpsApsAps(8nh)Gps(5m) |
| | | CpsGpsApsApsmoeGpsmoeTpsmoeGpsmoe(5m)CpsmoeA |
| ASO-203 | 204 | moe(5m)CpsmoeApsmoeGpsmoeApslnGpsGpsTpsGpsApsAps(8nh)Gps(5m) |
| | | CpsGpsApsApsmoeGpsmoeTpsmoeGpsmoe(5m)CpsmoeA |
| ASO-204 | 205 | moe(5m)CpsmoeApsmoeGpsmoeApsmoeGpsGpsTpsGpsApsAps(8nh)Gps |
| | | (5m)CpsGpsApsApslnGpsmoeTpsmoeGpsmoe(5m)CpsmoeA |
| ASO-205 | 206 | moe(5m)CpsmoeApsmoeGpsmoeApsmoeGpsGpsTpsGpsApsAps(8nh)Gps |
| | | (5m)CpsGpsApsApsmoeGpslnTpsmoeGpsmoe(5m)CpsmoeA |
| ASO-206 | 207 | moe(5m)CpsmoeApsmoeGpsmoeApsmoeGpsGpsTpsGpsApsAps(8nh)Gps |
| | | (5m)CpsGpsApsApsmoeGpsmoeTpslnGpsmoe(5m)CpsmoeA |
| ASO-207 | 208 | moe(5m)CpsmoeApsmoeGpsmoeApsmoeGpsGpsTpsGpsApsAps(8nh)Gps |
| | | (5m)CpsGpsApsApsmoeGpsmoeTpsmoeGpsln(5m)CpsmoeA |
| ASO-208 | 209 | moe(5m)CpsmoeApsmoeGpsmoeApsmoeGpsGpsTpsGpsApsAps(8nh)Gps |
| | | (5m)CpsGpsApsApsmoeGpsmoeTpsmoeGpsmoe(5m)CpslnA |
| ASO-209 | 210 | ln(5m)CpsmoeApsmoeGpsmoeApsmoeGpsGpsTpsGpsApsAps(8nh)Gps(5m) |
| | | CpsGpsApsApsmoeGpslnTpsmoeGpsmoe(5m)CpsmoeA |
| ASO-210 | 211 | ln(5m)CpsmoeApsmoeGpsmoeApsmoeGpsGpsTpsGpsApsAps(8nh)Gps(5m) |
| | | CpsGpsApsApsmoeGpsmoeTpsmoeGpsln(5m)CpsmoeA |
| ASO-211 | 212 | moe(5m)CpslnApsmoeGpslnApsmoeGpsGpsTpsGpsApsAps(8nh)Gps(5m) |
| | | CpsGpsApsApsmoeGpsmoeTpsmoeGpsmoe(5m)CpsmoeA |
| ASO-212 | 213 | moe(5m)CpslnApsmoeGpsmoeApsmoeGpsGpsTpsGpsApsAps(8nh)Gps(5m) |
| | | CpsGpsApsApsmoeGpsmoeTpsmoeGpsmoe(5m)CpslnA |
| ASO-213 | 214 | moe(5m)CpsmoeApsmoeGpslnApsmoeGpsGpsTpsGpsApsAps(8nh)Gps(5m) |
| | | CpsGpsApsApsmoeGpsmoeTpsmoeGpsmoe(5m)CpslnA |
| ASO-214 | 215 | moe(5m)CpsmoeApslnGpsmoeApslnGpsGpsTpsGpsApsAps(8nh)Gps(5m) |
| | | CpsGpsApsApsmoeGpsmoeTpsmoeGpsmoe(5m)CpsmoeA |
| ASO-215 | 216 | moe(5m)CpsmoeApsmoeGpsmoeApsmoeGpsGpsTpsGpsApsAps(8nh)Gps |
| | | (5m)CpsGpsApsApslnGpsmoeTpslnGpsmoe(5m)CpsmoeA |
| ASO-216 | 217 | moe(5m)CpsmoeApslnGpsmoeApsmoeGpsGpsTpsGpsApsAps(8nh)Gps(5m) |
| | | CpsGpsApsApslnGpsmoeTpsmoeGpsmoe(5m)CpsmoeA |
| ASO-217 | 218 | moe(5m)CpsmoeApslnGpsmoeApsmoeGpsGpsTpsGpsApsAps(8nh)Gps(5m) |
| | | CpsGpsApsApsmoeGpsmoeTpslnGpsmoe(5m)CpsmoeA |
| ASO-218 | 219 | moe(5m)CpsmoeApsmoeGpsmoeApslnGpsGpsTpsGpsApsAps(8nh)Gps(5m) |
| | | CpsGpsApsApslnGpsmoeTpsmoeGpsmoe(5m)CpsmoeA |
| ASO-219 | 220 | moe(5m)CpsmoeApsmoeGpsmoeApslnGpsGpsTpsGpsApsAps(8nh)Gps(5m) |
| | | CpsGpsApsApsmoeGpsmoeTpslnGpsmoe(5m)CpsmoeA |
| ASO-220 | 221 | moe(5m)CpslnApslnGpsmoeApslnGpsGpsTpsGpsApsAps(8nh)Gps(5m)Cps |
| | | GpsApsApsmoeGpsmoeTpsmoeGpsmoe(5m)CpsmoeA |
| ASO-221 | 222 | moe(5m)CpslnApsmoeGpslnApslnGpsGpsTpsGpsApsAps(8nh)Gps(5m)Cps |
| | | GpsApsApsmoeGpsmoeTpsmoeGpsmoe(5m)CpsmoeA |
| ASO-222 | 223 | moe(5m)CpsmoeApsmoeGpsmoeApsmoeGpsGpsTpsGpsApsAps(8nh)Gps |
| | | (5m)CpsGpsApsApslnGpsmoeTpslnGpsmoe(5m)CpslnA |
| ASO-223 | 224 | moe(5m)CpslnApslnGpsmoeApslnGpsGpsTpsGpsApsApsGps(5m)CpsGps |
| | | ApsApsmoeGpsmoeTpsmoeGpsmoe(5m)CpsmoeA |
| ASO-224 | 225 | moe(5m)CpslnApsmoeGpslnApslnGpsGpsTpsGpsApsApsGps(5m)CpsGps |
| | | ApsApsmoeGpsmoeTpsmoeGpsmoe(5m)CpsmoeA |
| ASO-225 | 226 | moe(5m)CpsmoeApsmoeGpsmoeApsmoeGpsGpsTpsGpsApsApsGps(5m)Cps |
| | | GpsApsApslnGpsmoeTpslnGpsmoe(5m)CpslnA |
| ASO-226 | 227 | moeGpsmoe(5m)CpsmoeApsmoeGpsApsGpsGpsTpsGpsmXpsApsGps(5m) |
| | | CpsGpsApsmoeApslnGpsmoeTpslnGpsln(5m)C |
| ASO-227 | 228 | moe(5m)CpsmoeApsmoeGpsmoeApsmoeGpsGpsr?TpsGpsApsApsGps(5m) |
| | | CpsGpsApsApsmoeGpsmoeTpsmoeGpsmoe(5m)CpsmoeA |
| ASO-228 | 229 | moe(5m)CpsmoeApsmoeGpsmoeApsmoeGpsGpsTpsr?GpsApsApsGps(5m) |
| | | CpsGpsApsApsmoeGpsmoeTpsmoeGpsmoe(5m)CpsmoeA |
| ASO-229 | 230 | moe(5m)CpsmoeApsmoeGpsmoeApsmoeGpsGpsTpsGpsr?ApsApsGps(5m) |
| | | CpsGpsApsApsmoeGpsmoeTpsmoeGpsmoe(5m)CpsmoeA |
| ASO-230 | 231 | moe(5m)CpsmoeApsmoeGpsmoeApsmoeGpsGpsTpsGpsApsr?ApsGps(5m) |
| | | CpsGpsApsApsmoeGpsmoeTpsmoeGpsmoe(5m)CpsmoeA |
| ASO-231 | 232 | moe(5m)CpsmoeApsmoeGpsmoeApsmoeGpsGpsTpsGpsApsApsr?Gps(5m) |
| | | CpsGpsApsApsmoeGpsmoeTpsmoeGpsmoe(5m)CpsmoeA |
| ASO-232 | 233 | moe(5m)CpsmoeApsmoeGpsmoeApsmoeGpsGpsTpsGpsApsApsGpsr?(5m) |
| | | CpsGpsApsApsmoeGpsmoeTpsmoeGpsmoe(5m)CpsmoeA |
| ASO-233 | 234 | moe(5m)CpsmoeApsmoeGpsmoeApsmoeGpsGpsTpsGpsApsApsGps(5m) |
| | | Cpsr?GpsApsApsmoeGpsmoeTpsmoeGpsmoe(5m)CpsmoeA |
| ASO-234 | 235 | moe(5m)CpsmoeApsmoeGpsmoeApsmoeGpsGpsTpsGpsApsApsGps(5m) |
| | | CpsGpsr?ApsApsmoeGpsmoeTpsmoeGpsmoe(5m)CpsmoeA |
| ASO-235 | 236 | moe(5m)CpsmoeApsmoeGpsmoeApsmoeGpsGpsTpsGpsApsApsGps(5m) |
| | | CpsGpsApsr?ApsmoeGpsmoeTpsmoeGpsmoe(5m)CpsmoeA |
| ASO-236 | 237 | moe(5m)CpsmoeApsmoeGpsmoeApsmoeGpsGpsTpsGpsApsApsGps(5m) |
| | | CpsGpsApsApsr?moeGpsmoeTpsmoeGpsmoe(5m)CpsmoeA |
| ASO-237 | 238 | moeApsmoeGpsmoeGpsmoeTpsmoeGps(8nh)ApsApsGps(5m)CpsGpsAps |
| | | ApsGpsTpsGpsmoe(5m)CpsmoeApsmoe(5m)CpsmoeApsmoe(5m)C |
| ASO-238 | 239 | moeApsmoeGpsmoeGpsmoeTpsmoeGpsAps(8nh)ApsGps(5m)CpsGpsAps |
| | | ApsGpsTpsGpsmoe(5m)CpsmoeApsmoe(5m)CpsmoeApsmoe(5m)C |
| ASO-239 | 240 | moeApsmoeGpsmoeGpsmoeTpsmoeGpsApsAps(8nh)Gps(5m)CpsGpsAps |
| | | ApsGpsTpsGpsmoe(5m)CpsmoeApsmoe(5m)CpsmoeApsmoe(5m)C |
| ASO-240 | 241 | moeApsmoeGpsmoeGpsmoeTpsmoeGpsApsApsGps(5oh)CpsGpsApsAps |
| | | GpsTpsGpsmoe(5m)CpsmoeApsmoe(5m)CpsmoeApsmoe(5m)C |
| ASO-241 | 242 | moeApsmoeGpsmoeGpsmoeTpsmoeGpsApsApsGps(5m)Cps(8nh)GpsAps |
| | | ApsGpsTpsGpsmoe(5m)CpsmoeApsmoe(5m)CpsmoeApsmoe(5m)C |
| ASO-242 | 243 | moeApsmoeGpsmoeGpsmoeTpsmoeGpsApsApsGps(5m)CpsGps(8nh)Aps |
| | | ApsGpsTpsGpsmoe(5m)CpsmoeApsmoe(5m)CpsmoeApsmoe(5m)C |
| ASO-243 | 244 | moeApsmoeGpsmoeGpsmoeTpsmoeGpsApsApsGps(5m)CpsGpsAps(8nh) |
| | | ApsGpsTpsGpsmoe(5m)CpsmoeApsmoe(5m)CpsmoeApsmoe(5m)C |
| ASO-244 | 245 | moeApsmoeGpsmoeGpsmoeTpsmoeGpsApsApsGps(5m)CpsGpsApsAps |
| | | (8nh)GpsTpsGpsmoe(5m)CpsmoeApsmoe(5m)CpsmoeApsmoe(5m)C |
| ASO-245 | 246 | moeApsmoeGpsmoeGpsmoeTpsmoeGpsApsApsGps(5m)CpsGpsApsAps |
| | | Gps(2s)TpsGpsmoe(5m)CpsmoeApsmoe(5m)CpsmoeApsmoe(5m)C |
| ASO-246 | 247 | moeApsmoeGpsmoeGpsmoeTpsmoeGpsApsApsGps(5m)CpsGpsApsAps |
| | | GpsTps(8nh)Gpsmoe(5m)CpsmoeApsmoe(5m)CpsmoeApsmoe(5m)C |
| ASO-247 | 248 | moe(5m)CpsmoeApsmoeGpsmoeApsmoeGpsGpss?Tpss?Gpsr?ApsApsGps |
| | | (5m)CpsGpsApsApsmoeGpsmoeTpsmoeGpsmoe(5m)CpsmoeA |
| ASO-248 | 249 | moe(5m)CpsmoeApsmoeGpsmoeApsmoeGpsGpsr?Tpsr?Gpss?ApsApsGps |
| | | (5m)CpsGpsApsApsmoeGpsmoeTpsmoeGpsmoe(5m)CpsmoeA |
| ASO-249 | 250 | moeApsmoe(5m)CpsmoeGpsmoeTpsmoeGps(5m)Cpss?ApsGpsApsGpsGps |
| | | TpsGpsApsApsmoeGpsmoe(5m)CpsmoeGpsmoeApsmoeA |
| ASO-250 | 251 | moeApsmoe(5m)CpsmoeGpsmoeTpsmoeGps(5m)CpsApss?GpsApsGpsGps |
| | | TpsGpsApsApsmoeGpsmoe(5m)CpsmoeGpsmoeApsmoeA |
| ASO-251 | 252 | moeApsmoe(5m)CpsmoeGpsmoeTpsmoeGps(5m)CpsApsGpss?ApsGpsGps |
| | | TpsGpsApsApsmoeGpsmoe(5m)CpsmoeGpsmoeApsmoeA |
| ASO-252 | 253 | moeApsmoe(5m)CpsmoeGpsmoeTpsmoeGps(5m)CpsApsGpsApss?GpsGps |
| | | TpsGpsApsApsmoeGpsmoe(5m)CpsmoeGpsmoeApsmoeA |
| ASO-253 | 254 | moeApsmoe(5m)CpsmoeGpsmoeTpsmoeGps(5m)CpsApsGpsApsGpss?Gps |
| | | TpsGpsApsApsmoeGpsmoe(5m)CpsmoeGpsmoeApsmoeA |
| ASO-254 | 255 | moeApsmoe(5m)CpsmoeGpsmoeTpsmoeGps(5m)CpsApsGpsApsGpsGpss? |
| | | TpsGpsApsApsmoeGpsmoe(5m)CpsmoeGpsmoeApsmoeA |
| ASO-255 | 256 | moeApsmoe(5m)CpsmoeGpsmoeTpsmoeGps(5m)CpsApsGpsApsGpsGps |
| | | Tpss?GpsApsApsmoeGpsmoe(5m)CpsmoeGpsmoeApsmoeA |
| ASO-256 | 257 | moeApsmoe(5m)CpsmoeGpsmoeTpsmoeGps(5m)CpsApsGpsApsGpsGps |
| | | TpsGpss?ApsApsmoeGpsmoe(5m)CpsmoeGpsmoeApsmoeA |
| ASO-257 | 258 | moeApsmoe(5m)CpsmoeGpsmoeTpsmoeGps(5m)CpsApsGpsApsGpsGps |
| | | TpsGpsApss?ApsmoeGpsmoe(5m)CpsmoeGpsmoeApsmoeA |
| ASO-258 | 259 | moeApsmoe(5m)CpsmoeGpsmoeTpsmoeGps(5m)CpsApsGpsApsGpsGps |
| | | TpsGpsApsApss?moeGpsmoe(5m)CpsmoeGpsmoeApsmoeA |
| ASO-259 | 260 | moeApsmoe(5m)CpsmoeGpsmoeTpsmoeGps(5m)Cpsr?ApsGpsApsGpsGps |
| | | TpsGpsApsApsmoeGpsmoe(5m)CpsmoeGpsmoeApsmoeA |
| ASO-260 | 261 | moeApsmoe(5m)CpsmoeGpsmoeTpsmoeGps(5m)CpsApsr?GpsApsGpsGps |
| | | TpsGpsApsApsmoeGpsmoe(5m)CpsmoeGpsmoeApsmoeA |
| ASO-261 | 262 | moeApsmoe(5m)CpsmoeGpsmoeTpsmoeGps(5m)CpsApsGpsr?ApsGpsGps |
| | | TpsGpsApsApsmoeGpsmoe(5m)CpsmoeGpsmoeApsmoeA |
| ASO-262 | 263 | moeApsmoe(5m)CpsmoeGpsmoeTpsmoeGps(5m)CpsApsGpsApsr?GpsGps |
| | | TpsGpsApsApsmoeGpsmoe(5m)CpsmoeGpsmoeApsmoeA |
| ASO-263 | 264 | moeApsmoe(5m)CpsmoeGpsmoeTpsmoeGps(5m)CpsApsGpsApsGpsr?Gps |
| | | TpsGpsApsApsmoeGpsmoe(5m)CpsmoeGpsmoeApsmoeA |
| ASO-264 | 265 | moeApsmoe(5m)CpsmoeGpsmoeTpsmoeGps(5m)CpsApsGpsApsGpsGpsr? |
| | | TpsGpsApsApsmoeGpsmoe(5m)CpsmoeGpsmoeApsmoeA |
| ASO-265 | 266 | moeApsmoe(5m)CpsmoeGpsmoeTpsmoeGps(5m)CpsApsGpsApsGpsGps |
| | | Tpsr?GpsApsApsmoeGpsmoe(5m)CpsmoeGpsmoeApsmoeA |
| ASO-266 | 267 | moeApsmoe(5m)CpsmoeGpsmoeTpsmoeGps(5m)CpsApsGpsApsGpsGps |
| | | TpsGpsr?ApsApsmoeGpsmoe(5m)CpsmoeGpsmoeApsmoeA |
| ASO-267 | 268 | moeApsmoe(5m)CpsmoeGpsmoeTpsmoeGps(5m)CpsApsGpsApsGpsGps |
| | | TpsGpsApsr?ApsmoeGpsmoe(5m)CpsmoeGpsmoeApsmoeA |
| ASO-268 | 269 | moeApsmoe(5m)CpsmoeGpsmoeTpsmoeGps(5m)CpsApsGpsApsGpsGps |
| | | TpsGpsApsApsr?moeGpsmoe(5m)CpsmoeGpsmoeApsmoeA |
| ASO-269 | 270 | moe(5m)CpsmoeApslnGpsmoeApslnGpsGpsTpsGpsApsApsGps(5m)CpsGps |
| | | ApsApsmoeGpsmoeTpslnGpsm(5m)CpsmA |
| ASO-270 | 271 | moe(5m)CpsmoeApsmoeGpsmoeApslnGpsGpsTpsGpsApsApsGps(5m)Cps |
| | | GpsApsApsmoeGpsmoeTpslnGpsm(5m)CpsmA |
| ASO-271 | 272 | moe(5m)CpsmoeApslnGpsmoeApsmoeGpsGpsTpsGpsApsApsGps(5m)Cps |
| | | GpsApsApsmoeGpsmoeTpslnGpsm(5m)CpsmA |
| ASO-272 | 273 | moe(5m)CpsmoeApsmoeGpsmoeApsmoeGpsGpsTpsGpsApsApsGps(5m) |
| | | CpsGpsApsApsmoeGpsmoeTpslnGpsm(5m)CpsmA |
| ASO-273 | 274 | moe(5m)CpsmoeApslnGpsmoeApslnGpsGpsTpsGpsApsApsGps(5m)Cps |
| | | GpsApsApsmoeGpsmoeTpsmGpsm(5m)CpsmA |
| ASO-274 | 275 | moe(5m)CpsmoeApsmoeGpsmoeApslnGpsGpsTpsGpsApsApsGps(5m)Cps |
| | | GpsApsApsmoeGpsmoeTpsmGpsm(5m)CpsmA |
| ASO-275 | 276 | moe(5m)CpsmoeApslnGpsmoeApsmoeGpsGpsTpsGpsApsApsGps(5m)Cps |
| | | GpsApsApsmoeGpsmoeTpsmGpsm(5m)CpsmA |
| ASO-276 | 277 | rGrCrCrCrGrUrCrUrGrUrUrGrUrGrUrGrArCrUrC |
| ASO-277 | 278 | moe(5m)CpsmoeApslnGpsmoeApsmoeGpsGpsTpsGpsApsApsGps(5m)Cps |
| | | GpsApsApsmGpsmoeTpsmGpsmoe(5m)CpsmA |
| ASO-278 | 279 | moe(5m)CpsmoeApsmoeGpsmoeApslnGpsGpsTpsGpsApsApsGps(5m)Cps |
| | | GpsApsApsmGpsmoeTpsmGpsmoe(5m)CpsmA |
| ASO-279 | 280 | moe(5m)CpsmoeApslnGpsmoeApslnGpsGpsTpsGpsApsApsGps(5m)CpsGps |
| | | ApsApsmGpsmoeTpsmGpsmoe(5m)CpsmA |
| ASO-280 | 281 | moe(5m)CpsmoeApsmoeGpsmoeApsmoeGpsGpsTpsGpsApsApsGps(5m) |
| | | CpsGpsApsApsmGpsmoeTpslnGpsmoe(5m)CpsmA |
| ASO-281 | 282 | moe(5m)CpsmoeApslnGpsmoeApsmoeGpsGpsTpsGpsApsApsGps(5m)Cps |
| | | GpsApsApsmGpsmoeTpslnGpsmoe(5m)CpsmA |
| ASO-282 | 283 | moe(5m)CpsmoeApsmoeGpsmoeApslnGpsGpsTpsGpsApsApsGps(5m)Cps |
| | | GpsApsApsmGpsmoeTpslnGpsmoe(5m)CpsmA |
| ASO-283 | 284 | moe(5m)CpsmoeApslnGpsmoeApslnGpsGpsTpsGpsApsApsGps(5m)CpsGps |
| | | ApsApsmGpsmoeTpslnGpsmoe(5m)CpsmA |
| ASO-284 | 285 | moeApsmoe(5m)CpsmGpsmoeTpsmGps(5m)CpsApsGpsApsGpsGpsTpsGps |
| | | ApsApsmoeGpsmoe(5m)CpsmoeGpsmoeApsmoeA |
| ASO-285 | 286 | moeApsmoe(5m)CpsmoeGpsmoeTpsmoeGps(5m)CpsApsGpsApsGpsGpsTps |
| | | GpsApsApsmGpsmoe(5m)CpsmGpsmoeApsmoeA |
| ASO-286 | 287 | moeApsmoe(5m)CpsmGpsmoeTpsmGps(5m)CpsApsGpsApsGpsGpsTpsGps |
| | | ApsApsmGpsmoe(5m)CpsmoeGpsmoeApsmoeA |
| ASO-287 | 288 | moeApsmoe(5m)CpsmGpsmoeTpsmGps(5m)CpsApsGpsApsGpsGpsTpsGps |
| | | ApsApsmoeGpsmoe(5m)CpsmGpsmoeApsmoeA |
| ASO-288 | 289 | moeApsmoe(5m)CpsmGpsmoeTpsmoeGps(5m)CpsApsGpsApsGpsGpsTps |
| | | GpsApsApsmGpsmoe(5m)CpsmGpsmoeApsmoeA |
| ASO-289 | 290 | moeApsmoe(5m)CpsmoeGpsmoeTpsmGps(5m)CpsApsGpsApsGpsGpsTps |
| | | GpsApsApsmGpsmoe(5m)CpsmGpsmoeApsmoeA |
| ASO-290 | 291 | moeApsmoe(5m)CpsmGpsmoeTpsmGps(5m)CpsApsGpsApsGpsGpsTpsGps |
| | | ApsApsmGpsmoe(5m)CpsmGpsmoeApsmoeA |
| ASO-291 | 292 | moeApsmoe(5m)CpsmoeGpsmoeTpsmoeGps(5m)CpsApsGpsApsGpsGps |
| | | TpsGpsApsmApsmGpsmoe(5m)CpsmoeGpsmoeApsmoeA |
| ASO-292 | 293 | moeApsmoe(5m)CpsmoeGpsmoeTpsmoeGps(5m)CpsApsGpsApsGpsGps |
| | | TpsGpsApsApsmGpsmoe(5m)CpsmGpsmApsmoeA |
| ASO-293 | 294 | moeApsmoe(5m)CpsmoeGpsmoeTpsmoeGps(5m)CpsApsGpsApsGpsGps |
| | | TpsGpsApsApsmGpsmoe(5m)CpsmGpsmoeApsmA |
| ASO-294 | 295 | moeApsmoe(5m)CpsmoeGpsmoeTpsmoeGps(5m)CpsApsGpsApsGpsGps |
| | | TpsGpsApsApsmGpsmoe(5m)CpsmoeGpsmApsmA |
| ASO-295 | 296 | moeApsmoe(5m)CpsmoeGpsmoeTpsmoeGps(5m)CpsApsGpsApsGpsGps |
| | | TpsGpsApsApsmoeGpsmoe(5m)CpsmGpsmApsmA |
| ASO-296 | 297 | moeApsmoe(5m)CpsmoeGpsmoeTpsmGpsm(5m)CpsApsGpsApsGpsGps |
| | | TpsGpsApsApsmoeGpsmoe(5m)CpsmoeGpsmoeApsmoeA |
| ASO-297 | 298 | moeApsmoe(5m)CpsmoeGpsmoeTpsmoeGps(5m)CpsApsGpsApsGpsGps |
| | | TpsGpsApsApsmGpsm(5m)CpsmoeGpsmoeApsmoeA |
| ASO-298 | 299 | moeApsmoe(5m)CpsmoeGpsmoeTpsmoeGps(5m)CpsApsGpsApsGpsGps |
| | | TpsGpsApsApsmGpsm(5m)CpsmGpsmoeApsmoeA |
| ASO-299 | 300 | moeApsmoe(5m)CpsmoeGpsmoeTpsmGpsm(5m)CpsmApsGpsApsGpsGps |
| | | TpsGpsApsApsmoeGpsmoe(5m)CpsmoeGpsmoeApsmoeA |
| ASO-300 | 301 | moe(5m)CpsmoeApsmoeGpsmoeApsmoeGpsocpGpsTpsGpsApsApsGps(5m) |
| | | CpsGpsApsApsmoeGpsmoeTpsmoeGpsmoe(5m)CpsmoeA |
| ASO-301 | 302 | moe(5m)CpsmoeApsmoeGpsmoeApsmoeGpsGpsocpUpsGpsApsApsGps(5m) |
| | | CpsGpsApsApsmoeGpsmoeTpsmoeGpsmoe(5m)CpsmoeA |
| ASO-302 | 303 | moe(5m)CpsmoeApsmoeGpsmoeApsmoeGpsGpsTpsocpGpsApsApsGps(5m) |
| | | CpsGpsApsApsmoeGpsmoeTpsmoeGpsmoe(5m)CpsmoeA |
| ASO-303 | 304 | moe(5m)CpsmoeApsmoeGpsmoeApsmoeGpsomcpGpsTpsGpsApsApsGps |
| | | (5m)CpsGpsApsApsmoeGpsmoeTpsmoeGpsmoe(5m)CpsmoeA |
| ASO-304 | 305 | moe(5m)CpsmoeApsmoeGpsmoeApsmoeGpsGpsomcpUpsGpsApsApsGps |
| | | (5m)CpsGpsApsApsmoeGpsmoeTpsmoeGpsmoe(5m)CpsmoeA |
| ASO-305 | 306 | moe(5m)CpsmoeApsmoeGpsmoeApsmoeGpsGpsTpsomcpGpsApsApsGps |
| | | (5m)CpsGpsApsApsmoeGpsmoeTpsmoeGpsmoe(5m)CpsmoeA |
| ASO-306 | 307 | moe(5m)CpsmoeApsmoeGpsmoeApsmoeGpsGpsTpsGpsApsApsGps(5m) |
| | | CpsGpsApsApsmoeGpsmoeTpsmoeGpsmoe(5m)CpsmoeA |
| ASO-307 | 308 | moe(5m)CpsmoeApsmoeGpsmoeApsmoeGpsGpslmUpsGpsApsApsGps(5m) |
| | | CpsGpsApsApsmoeGpsmoeTpsmoeGpsmoe(5m)CpsmoeA |
| ASO-308 | 309 | moe(5m)CpsmoeApsmoeGpsmoeApsmoeGpsGpsTpslmGpsApsApsGps(5m) |
| | | CpsGpsApsApsmoeGpsmoeTpsmoeGpsmoe(5m)CpsmoeA |
| ASO-309 | 310 | moeApsmoe(5m)CpsmoeGpsmoeTpsmoeGpsocpCpsApsGpsApsGpsGpsTps |
| | | GpsApsApsmoeGpsmoe(5m)CpsmoeGpsmoeApsmoeA |
| ASO-310 | 311 | moeApsmoe(5m)CpsmoeGpsmoeTpsmoeGps(5m)CpsocpApsGpsApsGpsGps |
| | | TpsGpsApsApsmoeGpsmoe(5m)CpsmoeGpsmoeApsmoeA |
| ASO-311 | 312 | moeApsmoe(5m)CpsmoeGpsmoeTpsmoeGps(5m)CpsApsocpGpsApsGpsGps |
| | | TpsGpsApsApsmoeGpsmoe(5m)CpsmoeGpsmoeApsmoeA |
| ASO-312 | 313 | moeApsmoe(5m)CpsmoeGpsmoeTpsmoeGpsomcpCpsApsGpsApsGpsGpsTps |
| | | GpsApsApsmoeGpsmoe(5m)CpsmoeGpsmoeApsmoeA |
| ASO-313 | 314 | moeApsmoe(5m)CpsmoeGpsmoeTpsmoeGps(5m)CpsomcpApsGpsApsGps |
| | | GpsTpsGpsApsApsmoeGpsmoe(5m)CpsmoeGpsmoeApsmoeA |
| ASO-314 | 315 | moeApsmoe(5m)CpsmoeGpsmoeTpsmoeGps(5m)CpsApsomcpGpsApsGps |
| | | GpsTpsGpsApsApsmoeGpsmoe(5m)CpsmoeGpsmoeApsmoeA |
| ASO-315 | 316 | moeApsmoe(5m)CpsmoeGpsmoeTpsmoeGpslmCpsApsGpsApsGpsGpsTps |
| | | GpsApsApsmoeGpsmoe(5m)CpsmoeGpsmoeApsmoeA |
| ASO-316 | 317 | moeApsmoe(5m)CpsmoeGpsmoeTpsmoeGps(5m)CpslmApsGpsApsGpsGps |
| | | TpsGpsApsApsmoeGpsmoe(5m)CpsmoeGpsmoeApsmoeA |
| ASO-317 | 318 | moeApsmoe(5m)CpsmoeGpsmoeTpsmoeGps(5m)CpsApslmGpsApsGpsGps |
| | | TpsGpsApsApsmoeGpsmoe(5m)CpsmoeGpsmoeApsmoeA |
| ASO-318 | 319 | moeGpsmoeApsmoeGpsmoeGpsmoeTpsGpsApsApsGps(5m)CpsGpsApsAps |
| | | GpsTpsmoeGps(moe5m)CpsmoeAps(moe5m)CpsmoeA |
| ASO-319 | 320 | moeGpsmoeTpsmoeGps(moe5m)CpsmoeApsGpsApsGpsGpsTpsGpsApsAps |
| | | Gps(5m)CpsmoeGpsmoeApsmoeApsmoeGpsmoeT |
| ASO-332 | 353 | mGpsmCpsmApsmCpsmUpsrUprCprGpdCprUprUpdCprAprCprCpsrUpsm |
| | | CpsrUpsmGpsmC |
| ASO-333 | 354 | mGpsmCpsmApsmCpsmUpsrUprUprGprUprUprUprUprAprUprUpsmUpsm |
| | | CpsmUpsmGpsmC |
| ASO-334 | 355 | mGpsmUpsmApsmUpsmUpsrUprUprGprUprUprUprUprAprUprUpsmUpsm |
| | | UpsmUpsmGpsmU |
| ASO-335 | 356 | mGpsmUpsmApsrUpsrUprUprUprGprUprUprUprUprAprUprUprUpsrUpsm |
| | | UpsmGpsmU |
| ASO-336 | 357 | mGpsmCpsmApsmCpsrUpsrUpdCprGpdCprUprUpdCprApdCprCpsrUpsm |
| | | CpsrUpsmGpsmC |
| ASO-337 | 358 | mGpsmCpsmApsmCpsrUpsrUpdCprGpdCprUprUpdCprApdCpdCpsrUpsm |
| | | CpsrUpsmGpsmC |
| ASO-338 | 359 | mGpsmCpsmApsmCprUprUpdCprGpdCprUprUpdCprApdCpdCprUpmCpsr |
| | | UpsmGpsmC |
| ASO-339 | 360 | mGpsmCpsmApsmCprUprUpdCprGpdCprUprUpdCprApdCpdCprUpmCpr |
| | | UpmGpsmC |
| ASO-343 | 364 | mGpsmCpsmApsmCpsmUpsrUprCprGprCprUprUprCprAprCprCpsrUpsm |
| | | CpsrUpsmGpsmC |
| ASO-344 | 365 | mGpsmCpsmApsmCpsrUpsrUprCprGprCprUprUprCprAprCprCpsrUpsmCpsr |
| | | UpsmGpsmC |
| ASO-345 | 366 | mGpsmCpsmApsmCprUprUprCprGprCprUprUprCprAprCprCpsrUpsmCpsr |
| | | UpsmGpsmC |
| ASO-346 | 367 | mGpsmCpsmApsmCprUprUprCprGprCprUprUprCprAprCprCprUpmCpsr |
| | | UpsmGpsmC |
| ASO-347 | 368 | mGpsmCpsmApsmCprUprUprCprGprCprUprUprCprAprCprCprUpmCprUpm |
| | | GpsmC |
| ASO-348 | 369 | mGpsmCpsmApsmCpsmUpsrUprUprGprCprUprUprCprAprCprCpsmUpsm |
| | | CpsmUpsmGpsmC |
| ASO-349 | 370 | mGpsmCpsmApsmCpsmUpsrUprCprGprUprUprUprCprAprCprCpsmUpsm |
| | | CpsmUpsmGpsmC |
| ASO-350 | 371 | mGpsmCpsmApsmCpsmUpsrUprCprGprCprUprUprCprAprCprUpsmUpsm |
| | | CpsmUpsmGpsmC |
| ASO-351 | 372 | mGpsmCpsmApsmUpsmUpsrUprCprGprCprUprUprCprAprCprCpsmUpsm |
| | | CpsmUpsmGpsmC |
| ASO-352 | 373 | mGpsmCpsmApsmCpsmUpsrUprCprGprCprUprUprCprAprCprCpsmUpsm |
| | | UpsmUpsmGpsmC |
| ASO-353 | 374 | mGpsmCpsmApsmCpsmUpsrUprUprGprCprUprUprCprAprCprUpsmUpsm |
| | | CpsmUpsmGpsmC |
| ASO-354 | 375 | mGpsmCpsmApsmCpsmUpsrUprUprGprUprUprUprCprAprCprCpsmUpsm |
| | | CpsmUpsmGpsmC |
| ASO-355 | 376 | mGpsmCpsmApsmUpsmUpsrUprCprGprCprUprUprCprAprCprCpsmUpsm |
| | | UpsmUpsmGpsmC |
| ASO-356 | 377 | rCprGprCprGprCprGprCprGprCprAprGprAprAprGprCprGprUprGprCprGprC |
| ASO-357 | 378 | rCprGprGprCprUprCprGprGprCprAprGprAprAprGprCprCprGprGprGprCprC |
| ASO-358 | 379 | rGprCprAprCprUprUprCprGprCprUprUprCprAprCprCprUprCprUprGprC |
| ASO-359 | 380 | rCprUprCprUprCprUprCprUprCprUprCprUprCprUprCprUprCprUprGprU |
| ASO-360 | 381 | rCprUprCprUprCprUprGprUprCprUprGprUprCprUprGprUprCprUprGprU |
| ASO-361 | 382 | rCprUprGprUprCprUprGprUprCprUprGprUprCprUprGprUprCprUprGprU |
| ASO-362 | 383 | rCprUprCprUprCprUprCprUprCprUprCprUprCprUprCprUprCprUpsrGprU |
| ASO-363 | 384 | rCprUprCprUprCprUpsrGprUprCprUpsrGprUprCprUpsrGprUprCprUpsrGprU |
| ASO-364 | 385 | rCprUpsrGprUprCprUpsrGprUprCprUpsrGprUprCprUpsrGprUprCprUpsrGprU |
| ASO-365 | 386 | rCprUprCprUprCprUprAprUprCprUprAprUprCprUprAprUprCprUprAprU |
| ASO-366 | 387 | rCprUprAprUprCprUprAprUprCprUprAprUprCprUprAprUprCprUprAprU |
| ASO-380 | 401 | mGpsmCpsmApsmCpsmUpsrUprCprGprCprUprUprCprAprCprCpsrUpsm |
| | | CpsrUpslmGpsmC |
| ASO-382 | 403 | rUprGprCprAprCprUprUprCprGprCprUprUprCprAprCprCprUprCprUprG |
| ASO-383 | 404 | rUprUprCprGprCprUprUprCprAprCprCprUprCprUprGprCprAprCprGprU |
| ASO-425 | 446 | moeGpsmoe(5m)Cpsmoe(5m)Cpsmoe(5m)CpsmoeGpsmoeTpsmoe(5m)Cps |
| | | moeTpsmoeGpsmoeTpsmoeTpsmoeGpsmoeTpsmoeGpsmoeTpsmoeGpsmoe |
| | | Apsmoe(5m)CpsmoeTpsmoe(5m)C |
| ASO-426 | 447 | moe(5m)CpsmoeApsmoeGpsmoeApsr?moeGpsGpsTpsGpsApsApsGps(5m) |
| | | CpsGpsApsApsmoeGpsmoeTpsmoeGpsmoe(5m)CpsmoeA |
| ASO-427 | 448 | moe(5m)CpsmoeApsmoeGpsr?moeApsmoeGpsGpsTpsGpsApsApsGps(5m) |
| | | CpsGpsApsApsmoeGpsmoeTpsmoeGpsmoe(5m)CpsmoeA |
| ASO-428 | 449 | moe(5m)CpsmoeApsr?moeGpsmoeApsmoeGpsGpsTpsGpsApsApsGps(5m) |
| | | CpsGpsApsApsmoeGpsmoeTpsmoeGpsmoe(5m)CpsmoeA |
| ASO-429 | 450 | moe(5m)Cpsr?moeApsmoeGpsmoeApsmoeGpsGpsTpsGpsApsApsGps(5m) |
| | | CpsGpsApsApsmoeGpsmoeTpsmoeGpsmoe(5m)CpsmoeA |
| ASO-430 | 451 | moe(5m)Cpsr?moeApsmoeGpsmoeApsr?moeGpsGpsTpsGpsApsApsGps(5m) |
| | | CpsGpsApsApsmoeGpsmoeTpsmoeGpsmoe(5m)CpsmoeA |
| ASO-431 | 452 | moe(5m)Cpsr?moeApsmoeGpsr?moeApsmoeGpsGpsTpsGpsApsApsGps(5m) |
| | | CpsGpsApsApsmoeGpsmoeTpsmoeGpsmoe(5m)CpsmoeA |
| ASO-432 | 453 | moe(5m)CpsmoeApsr?moeGpsmoeApsr?moeGpsGpsTpsGpsApsApsGps(5m) |
| | | CpsGpsApsApsmoeGpsmoeTpsmoeGpsmoe(5m)CpsmoeA |
| ASO-433 | 454 | moe(5m)CpsmoeApsmoeGpsmoeApsmoeGpsGpsTpsGpsApsApsd(8nh)Gps |
| | | (5m)CpsGpsApsApsmoeGpsmoeTpsmoeGpsocp(5m)CpsmoeA |
| ASO-434 | 455 | moe(5m)CpsmoeApsmoeGpsmoeApsmoeGpsGpsTpsGpsApsApsd(8nh)Gps |
| | | (5m)CpsGpsApsApsmoeGpsocpTpsmoeGpsocp(5m)CpsmoeA |
| ASO-435 | 456 | ocp(5m)CpsmoeApsmoeGpsmoeApsmoeGpsGpsTpsGpsApsApsd(8nh)Gps |
| | | (5m)CpsGpsApsApsmoeGpsocpTpsmoeGpsmoe(5m)CpsmoeA |
| ASO-436 | 457 | ocp(5m)CpsmoeApsmoeGpsmoeApsmoeGpsGpsTpsGpsApsApsd(8nh)Gps |
| | | (5m)CpsGpsApsApsmoeGpsmoeTpsmoeGpsocp(5m)CpsmoeA |
| ASO-437 | 458 | moe(5m)CpsocpApsmoeGpsocpApsmoeGpsGpsTpsGpsApsApsd(8nh)Gps |
| | | (5m)CpsGpsApsApsmoeGpsmoeTpsmoeGpsmoe(5m)CpsmoeA |
| ASO-438 | 459 | moe(5m)CpsocpApsmoeGpsmoeApsmoeGpsGpsTpsGpsApsApsd(8nh)Gps |
| | | (5m)CpsGpsApsApsmoeGpsmoeTpsmoeGpsmoe(5m)CpsocpA |
| ASO-439 | 460 | moe(5m)CpsmoeApsmoeGpsocpApsmoeGpsGpsTpsGpsApsApsd(8nh)Gps |
| | | (5m)CpsGpsApsApsmoeGpsmoeTpsmoeGpsmoe(5m)CpsocpA |
| ASO-440 | 461 | moe(5m)CpsocpApsmoeGpsocpApsmoeGpsGpsTpsGpsApsApsd(8nh)Gps |
| | | (5m)CpsGpsApsApsmoeGpsmoeTpsmoeGpsmoe(5m)CpsocpA |
| ASO-441 | 462 | moe(5m)CpsmoeApsocpGpsmoeApsocpGpsGpsTpsGpsApsApsd(8nh)Gps |
| | | (5m)CpsGpsApsApsmoeGpsmoeTpsmoeGpsmoe(5m)CpsmoeA |
| ASO-442 | 463 | moe(5m)CpsmoeApsmoeGpsmoeApsmoeGpsGpsTpsGpsApsApsd(8nh)Gps |
| | | (5m)CpsGpsApsApsocpGpsmoeTpsocpGpsmoe(5m)CpsmoeA |
| ASO-443 | 464 | moe(5m)CpsmoeApsocpGpsmoeApsmoeGpsGpsTpsGpsApsApsd(8nh)Gps |
| | | (5m)CpsGpsApsApsocpGpsmoeTpsmoeGpsmoe(5m)CpsmoeA |
| ASO-444 | 465 | moe(5m)CpsmoeApsocpGpsmoeApsmoeGpsGpsTpsGpsApsApsd(8nh)Gps |
| | | (5m)CpsGpsApsApsmoeGpsmoeTpsocpGpsmoe(5m)CpsmoeA |
| ASO-445 | 466 | moe(5m)CpsmoeApsmoeGpsmoeApsocpGpsGpsTpsGpsApsApsd(8nh)Gps |
| | | (5m)CpsGpsApsApsocpGpsmoeTpsmoeGpsmoe(5m)CpsmoeA |
| ASO-446 | 467 | moe(5m)CpsmoeApsmoeGpsmoeApsocpGpsGpsTpsGpsApsApsd(8nh)Gps |
| | | (5m)CpsGpsApsApsmoeGpsmoeTpsocpGpsmoe(5m)CpsmoeA |
| ASO-447 | 468 | moe(5m)CpsmoeApsocpGpsmoeApsocpGpsGpsTpsGpsApsApsd(8nh)Gps |
| | | (5m)CpsGpsApsApsocpGpsmoeTpsmoeGpsmoe(5m)CpsmoeA |
| ASO-448 | 469 | moe(5m)CpsmoeApsocpGpsmoeApsocpGpsGpsTpsGpsApsApsd(8nh)Gps |
| | | (5m)CpsGpsApsApsmoeGpsmoeTpsocpGpsmoe(5m)CpsmoeA |
| ASO-449 | 470 | moe(5m)CpsmoeApsocpGpsmoeApsmoeGpsGpsTpsGpsApsApsd(8nh)Gps |
| | | (5m)CpsGpsApsApsocpGpsmoeTpsocpGpsmoe(5m)CpsmoeA |
| ASO-450 | 471 | moe(5m)CpsmoeApsmoeGpsmoeApsocpGpsGpsTpsGpsApsApsd(8nh)Gps |
| | | (5m)CpsGpsApsApsocpGpsmoeTpsocpGpsmoe(5m)CpsmoeA |
| ASO-451 | 472 | moe(5m)CpsocpApsocpGpsmoeApsocpGpsGpsTpsGpsApsApsd(8nh)Gps |
| | | (5m)CpsGpsApsApsmoeGpsmoeTpsmoeGpsmoe(5m)CpsmoeA |
| ASO-452 | 473 | moe(5m)CpsocpApsmoeGpsocpApsocpGpsGpsTpsGpsApsApsd(8nh)Gps |
| | | (5m)CpsGpsApsApsmoeGpsmoeTpsmoeGpsmoe(5m)CpsmoeA |
| ASO-453 | 474 | moe(5m)CpsmoeApsmoeGpsmoeApsmoeGpsGpsTpsGpsApsApsd(8nh)Gps |
| | | (5m)CpsGpsApsApsocpGpsmoeTpsocpGpsmoe(5m)CpsocpA |
| ASO-454 | 475 | moe(5m)CpsmoeApsmoeGpsmoeApsmoeGpsGpsTpsGpsApsApsd(8nh)Gps |
| | | (5m)CpsGpsApsApsocpGpsocpTpsmoeGpsmoe(5m)CpsocpA |
| ASO-455 | 476 | moe(5m)CpsmoeApsmoeGpsmoeApsmoeGpsGpsTpsGpsApsApsd(8nh)Gps |
| | | (5m)CpsGpsApsApsocpGpsmoeTpsmoeGpsocp(5m)CpsocpA |
| ASO-456 | 477 | moe(5m)CpsmoeApsmoeGpsmoeApsmoeGpsGpsTpsGpsApsApsd(8nh)Gps |
| | | (5m)CpsGpsApsApsocpGpsmoeTpsocpGpsocp(5m)CpsmoeA |
| ASO-457 | 478 | moe(5m)CpsmoeApsmoeGpsmoeApsmoeGpsGpsTpsGpsApsApsd(8nh)Gps |
| | | (5m)CpsGpsApsApsocpGpsmoeTpslnGpsmoe(5m)CpslnA |
| ASO-458 | 479 | moe(5m)CpsmoeApsmoeGpsmoeApsmoeGpsGpsTpsGpsApsApsd(8nh)Gps |
| | | (5m)CpsGpsApsApslnGpsmoeTpsocpGpsmoe(5m)CpslnA |
| ASO-459 | 480 | moe(5m)CpsmoeApsmoeGpsmoeApsmoeGpsGpsTpsGpsApsApsd(8nh)Gps |
| | | (5m)CpsGpsApsApslnGpsmoeTpslnGpsmoe(5m)CpsocpA |
| ASO-460 | 481 | moe(5m)CpsmoeApsmoeGpsmoeApsmoeGpsGpsTpsGpsApsApsd(8nh)Gps |
| | | (5m)CpsGpsApsApsocpGpsmoeTpsocpGpsmoe(5m)CpslnA |
| ASO-461 | 482 | moe(5m)CpsmoeApsmoeGpsmoeApsmoeGpsGpsTpsGpsApsApsd(8nh)Gps |
| | | (5m)CpsGpsApsApslnGpsmoeTpsocpGpsmoe(5m)CpsocpA |
| ASO-462 | 483 | moe(5m)CpsmoeApsmoeGpsmoeApsmoeGpsGpsTpsGpsApsApsd(8nh)Gps |
| | | (5m)CpsGpsApsApsocpGpsmoeTpslnGpsmoe(5m)CpsocpA |
| ASO-463 | 484 | moe(5m)CpsmoeApsmoeGpsmoeApsmoeGpsGpsTpsGpsApsApsGps(5m)Cps |
| | | GpsApsApsmoeGpss?moeTpsmoeGpsmoe(5m)CpsmoeA |
| ASO-464 | 485 | moe(5m)CpsmoeApsmoeGpsmoeApsmoeGpsGpsTpsGpsApsApsGps(5m)Cps |
| | | GpsApsApsmoeGpsmoeTpss?moeGpsmoe(5m)CpsmoeA |
| ASO-465 | 486 | moe(5m)CpsmoeApsmoeGpsmoeApsmoeGpsGpsTpsGpsApsApsGps(5m)Cps |
| | | GpsApsApsmoeGpsmoeTpsmoeGpss?moe(5m)CpsmoeA |
| ASO-466 | 487 | moe(5m)CpsmoeApsmoeGpsmoeApsmoeGpsGpsTpsGpsApsApsGps(5m)Cps |
| | | GpsApsApsmoeGpsmoeTpsmoeGpsmoe(5m)Cpss?moeA |
| ASO-467 | 488 | moe(5m)CpsmoeApsmoeGpsmoeApsmoeGpsGpsTpsGpsApsApsGps(5m)Cps |
| | | GpsApsApsmoeGpsr?moeTpsmoeGpsmoe(5m)CpsmoeA |
| ASO-468 | 489 | moe(5m)CpsmoeApsmoeGpsmoeApsmoeGpsGpsTpsGpsApsApsGps(5m)Cps |
| | | GpsApsApsmoeGpsmoeTpsr?moeGpsmoe(5m)CpsmoeA |
| ASO-469 | 490 | moe(5m)CpsmoeApsmoeGpsmoeApsmoeGpsGpsTpsGpsApsApsGps(5m)Cps |
| | | GpsApsApsmoeGpsmoeTpsmoeGpsr?moe(5m)CpsmoeA |
| ASO-470 | 491 | ln(5m)CpsmoeApsmoeGpsmoeApsmoeGpsomcpGpsTpsGpsApsApsGps(5m) |
| | | CpsGpsApsApsmoeGpslnTpsmoeGpsmoe(5m)CpsmoeA |
| ASO-471 | 492 | ln(5m)CpsmoeApsmoeGpsmoeApsmoeGpsGpsTpsomcpGpsApsApsGps(5m) |
| | | CpsGpsApsApsmoeGpslnTpsmoeGpsmoe(5m)CpsmoeA |
| ASO-472 | 493 | ln(5m)CpsmoeApsmoeGpsmoeApsmoeGpsomcpGpsTpsomcpGpsApsApsGps |
| | | (5m)CpsGpsApsApsmoeGpslnTpsmoeGpsmoe(5m)CpsmoeA |
| ASO-473 | 494 | moe(5m)CpslnApsmoeGpslnApsmoeGpsomcpGpsTpsGpsApsApsGps(5m)Cps |
| | | GpsApsApsmoeGpsmoeTpsmoeGpsmoe(5m)CpsmoeA |
| ASO-474 | 495 | moe(5m)CpslnApsmoeGpslnApsmoeGpsGpsTpsomcpGpsApsApsGps(5m)Cps |
| | | GpsApsApsmoeGpsmoeTpsmoeGpsmoe(5m)CpsmoeA |
| ASO-475 | 496 | moe(5m)CpslnApsmoeGpslnApsmoeGpsomcpGpsTpsomcpGpsApsApsGps |
| | | (5m)CpsGpsApsApsmoeGpsmoeTpsmoeGpsmoe(5m)CpsmoeA |
| ASO-476 | 497 | moe(5m)CpsmoeApslnGpsmoeApsmoeGpsomcpGpsTpsGpsApsApsGps(5m) |
| | | CpsGpsApsApsmoeGpsmoeTpslnGpsmoe(5m)CpsmoeA |
| ASO-477 | 498 | moe(5m)CpsmoeApslnGpsmoeApsmoeGpsGpsTpsomcpGpsApsApsGps(5m) |
| | | CpsGpsApsApsmoeGpsmoeTpslnGpsmoe(5m)CpsmoeA |
| ASO-478 | 499 | moe(5m)CpsmoeApslnGpsmoeApsmoeGpsomcpGpsTpsomcpGpsApsApsGps |
| | | (5m)CpsGpsApsApsmoeGpsmoeTpslnGpsmoe(5m)CpsmoeA |
| ASO-479 | 500 | moe(5m)CpsmoeApsmoeGpsmoeApsmoeGpsGpsTpsGpsApsApsGps(5m)Cps |
| | | GpsApsApsmoeGpsmoeTpsmoeGpsocp(5m)CpsmoeA |
| ASO-480 | 501 | moe(5m)CpsmoeApsmoeGpsmoeApsmoeGpsGpsTpsGpsApsApsGps(5m)Cps |
| | | GpsApsApsmoeGpsocpTpsmoeGpsocp(5m)CpsmoeA |
| ASO-481 | 502 | ocp(5m)CpsmoeApsmoeGpsmoeApsmoeGpsGpsTpsGpsApsApsGps(5m)Cps |
| | | GpsApsApsmoeGpsocpTpsmoeGpsmoe(5m)CpsmoeA |
| ASO-482 | 503 | ocp(5m)CpsmoeApsmoeGpsmoeApsmoeGpsGpsTpsGpsApsApsGps(5m)Cps |
| | | GpsApsApsmoeGpsmoeTpsmoeGpsocp(5m)CpsmoeA |
| ASO-483 | 504 | moe(5m)CpsocpApsmoeGpsocpApsmoeGpsGpsTpsGpsApsApsGps(5m)Cps |
| | | GpsApsApsmoeGpsmoeTpsmoeGpsmoe(5m)CpsmoeA |
| ASO-484 | 505 | moe(5m)CpsocpApsmoeGpsmoeApsmoeGpsGpsTpsGpsApsApsGps(5m)Cps |
| | | GpsApsApsmoeGpsmoeTpsmoeGpsmoe(5m)CpsocpA |
| ASO-485 | 506 | moe(5m)CpsmoeApsmoeGpsocpApsmoeGpsGpsTpsGpsApsApsGps(5m)Cps |
| | | GpsApsApsmoeGpsmoeTpsmoeGpsmoe(5m)CpsocpA |
| ASO-486 | 507 | moe(5m)CpsmoeApsocpGpsmoeApsocpGpsGpsTpsGpsApsApsGps(5m)Cps |
| | | GpsApsApsmoeGpsmoeTpsmoeGpsmoe(5m)CpsmoeA |
| ASO-487 | 508 | moe(5m)CpsmoeApsmoeGpsmoeApsmoeGpsGpsTpsGpsApsApsGps(5m)Cps |
| | | GpsApsApsocpGpsmoeTpsocpGpsmoe(5m)CpsmoeA |
| ASO-488 | 509 | moe(5m)CpsmoeApsocpGpsmoeApsmoeGpsGpsTpsGpsApsApsGps(5m)Cps |
| | | GpsApsApsocpGpsmoeTpsmoeGpsmoe(5m)CpsmoeA |
| ASO-489 | 510 | moe(5m)CpsmoeApsocpGpsmoeApsmoeGpsGpsTpsGpsApsApsGps(5m)Cps |
| | | GpsApsApsmoeGpsmoeTpsocpGpsmoe(5m)CpsmoeA |
| ASO-490 | 511 | moe(5m)CpsmoeApsmoeGpsmoeApsocpGpsGpsTpsGpsApsApsGps(5m)Cps |
| | | GpsApsApsocpGpsmoeTpsmoeGpsmoe(5m)CpsmoeA |
| ASO-491 | 512 | moe(5m)CpsmoeApsmoeGpsmoeApsocpGpsGpsTpsGpsApsApsGps(5m)Cps |
| | | GpsApsApsmoeGpsmoeTpsocpGpsmoe(5m)CpsmoeA |
| ASO-492 | 513 | moe(5m)CpsocpApsocpGpsmoeApsocpGpsGpsTpsGpsApsApsGps(5m)Cps |
| | | GpsApsApsmoeGpsmoeTpsmoeGpsmoe(5m)CpsmoeA |
| ASO-493 | 514 | moe(5m)CpsocpApsmoeGpsocpApsocpGpsGpsTpsGpsApsApsGps(5m)Cps |
| | | GpsApsApsmoeGpsmoeTpsmoeGpsmoe(5m)CpsmoeA |
| ASO-494 | 515 | moe(5m)CpsmoeApsmoeGpsmoeApsmoeGpsGpsTpsGpsApsApsGps(5m)Cps |
| | | GpsApsApsocpGpsmoeTpsocpGpsmoe(5m)CpsocpA |
| ASO-495 | 516 | moe(5m)CpsmoeApsmoeGpsmoeApsmoeGpsGpsTpsGpsApsApsGps(5m)Cps |
| | | GpsApsApsocpGpsocpTpsmoeGpsmoe(5m)CpsocpA |
| ASO-496 | 517 | moe(5m)CpsmoeApsmoeGpsmoeApsmoeGpsGpsTpsGpsApsApsGps(5m)Cps |
| | | GpsApsApsocpGpsmoeTpsmoeGpsocp(5m)CpsocpA |
| ASO-497 | 518 | moe(5m)CpsmoeApsmoeGpsmoeApsmoeGpsGpsTpsGpsApsApsGps(5m)Cps |
| | | GpsApsApsocpGpsmoeTpsocpGpsocp(5m)CpsmoeA |
| ASO-498 | 519 | moe(5m)CpsocpApsmoeGpsocpApsmoeGpsGpsTpsGpsApsApsGps(5m)Cps |
| | | GpsApsApsmoeGpsmoeTpsmoeGpsmoe(5m)CpsocpA |
| ASO-499 | 520 | moe(5m)CpsmoeApsocpGpsmoeApsocpGpsGpsTpsGpsApsApsGps(5m)Cps |
| | | GpsApsApsocpGpsmoeTpsmoeGpsmoe(5m)CpsmoeA |
| ASO-500 | 521 | moe(5m)CpsmoeApsocpGpsmoeApsocpGpsGpsTpsGpsApsApsGps(5m)Cps |
| | | GpsApsApsmoeGpsmoeTpsocpGpsmoe(5m)CpsmoeA |
| ASO-501 | 522 | moe(5m)CpsmoeApsocpGpsmoeApsmoeGpsGpsTpsGpsApsApsGps(5m)Cps |
| | | GpsApsApsocpGpsmoeTpsocpGpsmoe(5m)CpsmoeA |
| ASO-502 | 523 | moe(5m)CpsmoeApsmoeGpsmoeApsocpGpsGpsTpsGpsApsApsGps(5m)Cps |
| | | GpsApsApsocpGpsmoeTpsocpGpsmoe(5m)CpsmoeA |
| ASO-503 | 524 | moe(5m)CpsmoeApsmoeGpsmoeApsmoeGpsGpsTpsGpsApsApsGps(5m)Cps |
| | | GpsApsApsocpGpsmoeTpslnGpsmoe(5m)CpslnA |
| ASO-504 | 525 | moe(5m)CpsmoeApsmoeGpsmoeApsmoeGpsGpsTpsGpsApsApsGps(5m)Cps |
| | | GpsApsApslnGpsmoeTpsocpGpsmoe(5m)CpslnA |
| ASO-505 | 526 | moe(5m)CpsmoeApsmoeGpsmoeApsmoeGpsGpsTpsGpsApsApsGps(5m)Cps |
| | | GpsApsApslnGpsmoeTpslnGpsmoe(5m)CpsocpA |
| ASO-506 | 527 | moe(5m)CpsmoeApsmoeGpsmoeApsmoeGpsGpsTpsGpsApsApsGps(5m)Cps |
| | | GpsApsApsocpGpsmoeTpsocpGpsmoe(5m)CpslnA |
| ASO-507 | 528 | moe(5m)CpsmoeApsmoeGpsmoeApsmoeGpsGpsTpsGpsApsApsGps(5m)Cps |
| | | GpsApsApslnGpsmoeTpsocpGpsmoe(5m)CpsocpA |
| ASO-508 | 529 | moe(5m)CpsmoeApsmoeGpsmoeApsmoeGpsGpsTpsGpsApsApsGps(5m)Cps |
| | | GpsApsApsocpGpsmoeTpslnGpsmoe(5m)CpsocpA |
| ASO-509 | 530 | moeApsmoeGpsmoeGpslnTpsmoeGpsApsApsGps(5m)CpsGpsApsApsGpsTps |
| | | Gpsln(5m)CpsmoeApsmoe(5m)CpsmoeApsmoe(5m)C |
| ASO-510 | 531 | moeApsmoeGpsmoeGpslnTpsmoeGpsApsApsGps(5m)CpsGpsApsApsGpsTps |
| | | Gpsmoe(5m)CpsmoeApsln(5m)CpsmoeApsmoe(5m)C |
| ASO-511 | 532 | moeApsmoeGpsmoeGpslnTpsmoeGpsApsApsGps(5m)CpsGpsApsApsGpsTps |
| | | Gpsmoe(5m)CpsmoeApsmoe(5m)CpsmoeApsln(5m)C |
| ASO-512 | 533 | moeApsmoeGpsmoeGpsmoeTpsmoeGpsApsApsGps(5m)CpsGpsApsApsGps |
| | | TpsGpsln(5m)CpsmoeApsln(5m)CpsmoeApsmoe(5m)C |
| ASO-513 | 534 | moeApsmoeGpsmoeGpsmoeTpsmoeGpsApsApsGps(5m)CpsGpsApsApsGps |
| | | TpsGpsln(5m)CpsmoeApsmoe(5m)CpsmoeApsln(5m)C |
| ASO-514 | 535 | moeApsmoeGpsmoeGpsmoeTpsmoeGpsApsApsGps(5m)CpsGpsApsApsGps |
| | | TpsGpsmoe(5m)CpsmoeApsln(5m)CpsmoeApsln(5m)C |
| ASO-515 | 536 | lnApsmoeGpsmoeGpsmoeTpsmoeGpsApsApsGps(5m)CpsGpsApsApsGpsTps |
| | | Gpsmoe(5m)CpslnApsmoe(5m)CpsmoeApsmoe(5m)C |
| ASO-516 | 537 | lnApsmoeGpsmoeGpsmoeTpsmoeGpsApsApsGps(5m)CpsGpsApsApsGpsTps |
| | | Gpsmoe(5m)CpsmoeApsmoe(5m)CpslnApsmoe(5m)C |
| ASO-517 | 538 | moeApslnGpslnGpsmoeTpsmoeGpsApsApsGps(5m)CpsGpsApsApsGpsTps |
| | | Gpsmoe(5m)CpsmoeApsmoe(5m)CpsmoeApsmoe(5m)C |
| ASO-518 | 539 | moeApslnGpsmoeGpsmoeTpslnGpsApsApsGps(5m)CpsGpsApsApsGpsTps |
| | | Gpsmoe(5m)CpsmoeApsmoe(5m)CpsmoeApsmoe(5m)C |
| ASO-519 | 540 | moeApsmoeGpslnGpsmoeTpslnGpsApsApsGps(5m)CpsGpsApsApsGpsTps |
| | | Gpsmoe(5m)CpsmoeApsmoe(5m)CpsmoeApsmoe(5m)C |
| ASO-520 | 541 | lnApslnGpslnGpsmoeTpsmoeGpsApsApsGps(5m)CpsGpsApsApsGpsTpsGps |
| | | moe(5m)CpsmoeApsmoe(5m)CpsmoeApsmoe(5m)C |
| ASO-521 | 542 | lnApslnGpsmoeGpsmoeTpslnGpsApsApsGps(5m)CpsGpsApsApsGpsTpsGps |
| | | moe(5m)CpsmoeApsmoe(5m)CpsmoeApsmoe(5m)C |
| ASO-522 | 543 | moeApslnGpslnGpsmoeTpslnGpsApsApsGps(5m)CpsGpsApsApsGpsTpsGps |
| | | moe(5m)CpsmoeApsmoe(5m)CpsmoeApsmoe(5m)C |
| ASO-523 | 544 | lnApsmoeGpslnGpsmoeTpslnGpsApsApsGps(5m)CpsGpsApsApsGpsTpsGps |
| | | moe(5m)CpsmoeApsmoe(5m)CpsmoeApsmoe(5m)C |
| ASO-524 | 545 | moeApsmoeGpsmoeGpsmoeTpsmoeGpsApsApsGps(5m)CpsGpsApsApsGps |
| | | TpsGpsln(5m)CpslnApsmoe(5m)CpslnApsmoe(5m)C |
| ASO-525 | 546 | moeApsmoeGpsmoeGpsmoeTpsmoeGpsApsApsGps(5m)CpsGpsApsApsGps |
| | | TpsGpsln(5m)CpslnApsmoe(5m)CpsmoeApsln(5m)C |
| ASO-526 | 547 | moeApsmoeGpsmoeGpsmoeTpsmoeGpsApsApsGps(5m)CpsGpsApsApsGps |
| | | TpsGpsln(5m)CpsmoeApsln(5m)CpslnApsmoe(5m)C |
| ASO-527 | 548 | moeApsmoeGpsmoeGpsmoeTpsmoeGpsApsApsGps(5m)CpsGpsApsApsGps |
| | | TpsGpsln(5m)CpsmoeApsln(5m)CpsmoeApsln(5m)C |
| ASO-528 | 549 | moeApsmoeGpsmoeGpsmoeTpsmoeGpsApsApsGps(5m)CpsGpsApsApsGps |
| | | TpsGpsmoe(5m)CpslnApsln(5m)CpsmoeApsln(5m)C |
| ASO-529 | 550 | lnApsmoeGpsmoeGpsmoeTpsmoeGpsApsApsGps(5m)CpsGpsApsApsGpsTps |
| | | Gpsmoe(5m)CpslnApsmoe(5m)CpslnApsmoe(5m)C |
| ASO-530 | 551 | moeApsmoeGpsmoeGpslnTpsmoeGpsApsApsGps(5m)CpsGpsApsApsGpsTps |
| | | Gpsln(5m)CpsmoeApsln(5m)CpsmoeApsmoe(5m)C |
| ASO-531 | 552 | moeApsmoeGpsmoeGpslnTpsmoeGpsApsApsGps(5m)CpsGpsApsApsGpsTps |
| | | Gpsln(5m)CpsmoeApsmoe(5m)CpsmoeApsln(5m)C |
| ASO-532 | 553 | moeApsmoeGpsmoeGpslnTpsmoeGpsApsApsGps(5m)CpsGpsApsApsGpsTps |
| | | Gpsmoe(5m)CpsmoeApsln(5m)CpsmoeApsln(5m)C |
| ASO-533 | 554 | moeApsmoeGpsmoeGpsmoeTpslnGpsApsApsGps(5m)CpsGpsApsApsGpsTps |
| | | Gpsln(5m)CpsmoeApsln(5m)CpsmoeApsmoe(5m)C |
| ASO-534 | 555 | moeApsmoeGpsmoeGpsmoeTpslnGpsApsApsGps(5m)CpsGpsApsApsGpsTps |
| | | Gpsln(5m)CpsmoeApsmoe(5m)CpsmoeApsln(5m)C |
| ASO-535 | 556 | moeApsmoeGpsmoeGpsmoeTpslnGpsApsApsGps(5m)CpsGpsApsApsGpsTps |
| | | Gpsmoe(5m)CpsmoeApsln(5m)CpsmoeApsln(5m)C |
| ASO-536 | 557 | ln(5m)CpsmoeApsmoeGpsmoeApsmoeGpsd(8nh)GpsTpsGpsApsApsGps(5m) |
| | | CpsGpsApsApsmoeGpslnTpsmoeGpsmoe(5m)CpsmoeA |
| ASO-537 | 558 | ln(5m)CpsmoeApsmoeGpsmoeApsmoeGpsGpsd(2s)TpsGpsApsApsGps(5m) |
| | | CpsGpsApsApsmoeGpslnTpsmoeGpsmoe(5m)CpsmoeA |
| ASO-538 | 559 | ln(5m)CpsmoeApsmoeGpsmoeApsmoeGpsGpsTpsd(8nh)GpsApsApsGps |
| | | (5m)CpsGpsApsApsmoeGpslnTpsmoeGpsmoe(5m)CpsmoeA |
| ASO-539 | 560 | ln(5m)CpsmoeApsmoeGpsmoeApsmoeGpsGpsTpsGpsd(8nh)ApsApsGps |
| | | (5m)CpsGpsApsApsmoeGpslnTpsmoeGpsmoe(5m)CpsmoeA |
| ASO-540 | 561 | ln(5m)CpsmoeApsmoeGpsmoeApsmoeGpsGpsTpsGpsApsd(8nh)ApsGps |
| | | (5m)CpsGpsApsApsmoeGpslnTpsmoeGpsmoe(5m)CpsmoeA |
| ASO-541 | 562 | ln(5m)CpsmoeApsmoeGpsmoeApsmoeGpsGpsTpsGpsApsApsGpsd(5oh)Cps |
| | | GpsApsApsmoeGpslnTpsmoeGpsmoe(5m)CpsmoeA |
| ASO-542 | 563 | ln(5m)CpsmoeApsmoeGpsmoeApsmoeGpsGpsTpsGpsApsApsGps(5m)Cpsd |
| | | (8nh)GpsApsApsmoeGpslnTpsmoeGpsmoe(5m)CpsmoeA |
| ASO-543 | 564 | ln(5m)CpsmoeApsmoeGpsmoeApsmoeGpsGpsTpsGpsApsApsGps(5m)Cps |
| | | Gpsd(8nh)ApsApsmoeGpslnTpsmoeGpsmoe(5m)CpsmoeA |
| ASO-544 | 565 | ln(5m)CpsmoeApsmoeGpsmoeApsmoeGpsGpsTpsGpsApsApsGps(5m)Cps |
| | | GpsApsd(8nh)ApsmoeGpslnTpsmoeGpsmoe(5m)CpsmoeA |
| ASO-545 | 566 | ln(5m)CpslnApsmoeGpsmoeApsmoeGpsGpsTpsGpsApsApsGps(5m)CpsGps |
| | | ApsApsmoeGpsmoeTpsmoeGpsmoe(5m)CpsmoeA |
| ASO-546 | 567 | ln(5m)CpsmoeApslnGpsmoeApsmoeGpsGpsTpsGpsApsApsGps(5m)CpsGps |
| | | ApsApsmoeGpsmoeTpsmoeGpsmoe(5m)CpsmoeA |
| ASO-547 | 568 | ln(5m)CpsmoeApsmoeGpslnApsmoeGpsGpsTpsGpsApsApsGps(5m)CpsGps |
| | | ApsApsmoeGpsmoeTpsmoeGpsmoe(5m)CpsmoeA |
| ASO-548 | 569 | ln(5m)CpsmoeApsmoeGpsmoeApslnGpsGpsTpsGpsApsApsGps(5m)CpsGps |
| | | ApsApsmoeGpsmoeTpsmoeGpsmoe(5m)CpsmoeA |
| ASO-549 | 570 | ln(5m)CpsmoeApsmoeGpsmoeApsmoeGpsGpsTpsGpsApsApsGps(5m)Cps |
| | | GpsApsApslnGpsmoeTpsmoeGpsmoe(5m)CpsmoeA |
| ASO-550 | 571 | ln(5m)CpsmoeApsmoeGpsmoeApsmoeGpsGpsTpsGpsApsApsGps(5m)Cps |
| | | GpsApsApsmoeGpsmoeTpslnGpsmoe(5m)CpsmoeA |
| ASO-551 | 572 | ln(5m)CpsmoeApsmoeGpsmoeApsmoeGpsGpsTpsGpsApsApsGps(5m)Cps |
| | | GpsApsApsmoeGpsmoeTpsmoeGpsmoe(5m)CpslnA |
| ASO-552 | 573 | moe(5m)CpslnApslnGpsmoeApsmoeGpsGpsTpsGpsApsApsGps(5m)CpsGps |
| | | ApsApsmoeGpsmoeTpsmoeGpsmoe(5m)CpsmoeA |
| ASO-553 | 574 | moe(5m)CpslnApsmoeGpsmoeApslnGpsGpsTpsGpsApsApsGps(5m)CpsGps |
| | | ApsApsmoeGpsmoeTpsmoeGpsmoe(5m)CpsmoeA |
| ASO-554 | 575 | moe(5m)CpslnApsmoeGpsmoeApsmoeGpsGpsTpsGpsApsApsGps(5m)Cps |
| | | GpsApsApslnGpsmoeTpsmoeGpsmoe(5m)CpsmoeA |
| ASO-555 | 576 | moe(5m)CpslnApsmoeGpsmoeApsmoeGpsGpsTpsGpsApsApsGps(5m)Cps |
| | | GpsApsApsmoeGpslnTpsmoeGpsmoe(5m)CpsmoeA |
| ASO-556 | 577 | moe(5m)CpslnApsmoeGpsmoeApsmoeGpsGpsTpsGpsApsApsGps(5m)Cps |
| | | GpsApsApsmoeGpsmoeTpslnGpsmoe(5m)CpsmoeA |
| ASO-557 | 578 | moe(5m)CpslnApsmoeGpsmoeApsmoeGpsGpsTpsGpsApsApsGps(5m)Cps |
| | | GpsApsApsmoeGpsmoeTpsmoeGpsln(5m)CpsmoeA |
| ASO-558 | 579 | moe(5m)CpsmoeApslnGpslnApsmoeGpsGpsTpsGpsApsApsGps(5m)CpsGps |
| | | ApsApsmoeGpsmoeTpsmoeGpsmoe(5m)CpsmoeA |
| ASO-559 | 580 | moe(5m)CpsmoeApslnGpsmoeApsmoeGpsGpsTpsGpsApsApsGps(5m)Cps |
| | | GpsApsApsmoeGpslnTpsmoeGpsmoe(5m)CpsmoeA |
| ASO-560 | 581 | moe(5m)CpsmoeApslnGpsmoeApsmoeGpsGpsTpsGpsApsApsGps(5m)Cps |
| | | GpsApsApsmoeGpsmoeTpsmoeGpsln(5m)CpsmoeA |
| ASO-561 | 582 | moe(5m)CpsmoeApslnGpsmoeApsmoeGpsGpsTpsGpsApsApsGps(5m)Cps |
| | | GpsApsApsmoeGpsmoeTpsmoeGpsmoe(5m)CpslnA |
| ASO-562 | 583 | moe(5m)CpsmoeApsmoeGpslnApslnGpsGpsTpsGpsApsApsGps(5m)CpsGps |
| | | ApsApsmoeGpsmoeTpsmoeGpsmoe(5m)CpsmoeA |
| ASO-563 | 584 | moe(5m)CpsmoeApsmoeGpslnApsmoeGpsGpsTpsGpsApsApsGps(5m)Cps |
| | | GpsApsApslnGpsmoeTpsmoeGpsmoe(5m)CpsmoeA |
| ASO-564 | 585 | moe(5m)CpsmoeApsmoeGpslnApsmoeGpsGpsTpsGpsApsApsGps(5m)Cps |
| | | GpsApsApsmoeGpslnTpsmoeGpsmoe(5m)CpsmoeA |
| ASO-565 | 586 | moe(5m)CpsmoeApsmoeGpslnApsmoeGpsGpsTpsGpsApsApsGps(5m)Cps |
| | | GpsApsApsmoeGpsmoeTpslnGpsmoe(5m)CpsmoeA |
| ASO-566 | 587 | moe(5m)CpsmoeApsmoeGpslnApsmoeGpsGpsTpsGpsApsApsGps(5m)Cps |
| | | GpsApsApsmoeGpsmoeTpsmoeGpsln(5m)CpsmoeA |
| ASO-567 | 588 | moe(5m)CpsmoeApsmoeGpsmoeApslnGpsGpsTpsGpsApsApsGps(5m)Cps |
| | | GpsApsApsmoeGpslnTpsmoeGpsmoe(5m)CpsmoeA |
| ASO-568 | 589 | moe(5m)CpsmoeApsmoeGpsmoeApslnGpsGpsTpsGpsApsApsGps(5m)Cps |
| | | GpsApsApsmoeGpsmoeTpsmoeGpsln(5m)CpsmoeA |
| ASO-569 | 590 | moe(5m)CpsmoeApsmoeGpsmoeApslnGpsGpsTpsGpsApsApsGps(5m)Cps |
| | | GpsApsApsmoeGpsmoeTpsmoeGpsmoe(5m)CpslnA |
| ASO-570 | 591 | moe(5m)CpsmoeApsmoeGpsmoeApsmoeGpsGpsTpsGpsApsApsGps(5m)Cps |
| | | GpsApsApslnGpslnTpsmoeGpsmoe(5m)CpsmoeA |
| ASO-571 | 592 | moe(5m)CpsmoeApsmoeGpsmoeApsmoeGpsGpsTpsGpsApsApsGps(5m)Cps |
| | | GpsApsApslnGpsmoeTpsmoeGpsln(5m)CpsmoeA |
| ASO-572 | 593 | moe(5m)CpsmoeApsmoeGpsmoeApsmoeGpsGpsTpsGpsApsApsGps(5m)Cps |
| | | GpsApsApslnGpsmoeTpsmoeGpsmoe(5m)CpslnA |
| ASO-573 | 594 | moe(5m)CpsmoeApsmoeGpsmoeApsmoeGpsGpsTpsGpsApsApsGps(5m)Cps |
| | | GpsApsApsmoeGpslnTpslnGpsmoe(5m)CpsmoeA |
| ASO-574 | 595 | moe(5m)CpsmoeApsmoeGpsmoeApsmoeGpsGpsTpsGpsApsApsGps(5m)Cps |
| | | GpsApsApsmoeGpslnTpsmoeGpsln(5m)CpsmoeA |
| ASO-575 | 596 | moe(5m)CpsmoeApsmoeGpsmoeApsmoeGpsGpsTpsGpsApsApsGps(5m)Cps |
| | | GpsApsApsmoeGpslnTpsmoeGpsmoe(5m)CpslnA |
| ASO-576 | 597 | moe(5m)CpsmoeApsmoeGpsmoeApsmoeGpsGpsTpsGpsApsApsGps(5m)Cps |
| | | GpsApsApsmoeGpsmoeTpslnGpsln(5m)CpsmoeA |
| ASO-577 | 598 | moe(5m)CpsmoeApsmoeGpsmoeApsmoeGpsGpsTpsGpsApsApsGps(5m)Cps |
| | | GpsApsApsmoeGpsmoeTpslnGpsmoe(5m)CpslnA |
| ASO-578 | 599 | moe(5m)CpsmoeApsmoeGpsmoeApsmoeGpsGpsTpsGpsApsApsGps(5m)Cps |
| | | GpsApsApsmoeGpsmoeTpsmoeGpsln(5m)CpslnA |
| ASO-579 | 600 | moe(5m)CpsmoeApsmoeGpsmoeApsmoeGpsGypTpsGpsApsApsGps(5m)Cps |
| | | GpsApsApsmoeGpsmoeTpsmoeGpsmoe(5m)CpsmoeA |
| ASO-580 | 601 | moe(5m)CpsmoeApsmoeGpsmoeApsmoeGypdGypTpsGpsApsApsGps(5m) |
| | | CpsGpsApsApsmoeGpsmoeTpsmoeGpsmoe(5m)CpsmoeA |
| ASO-581 | 602 | moe(5m)CpsmoeApsmoeGpsmoeApsmoeGpsGypdTypGpsApsApsGps(5m) |
| | | CpsGpsApsApsmoeGpsmoeTpsmoeGpsmoe(5m)CpsmoeA |
| ASO-582 | 603 | moe(5m)CpsmoeApsmoeGpsmoeApsmoeGpsGpsTypdGypApsApsGps(5m) |
| | | CpsGpsApsApsmoeGpsmoeTpsmoeGpsmoe(5m)CpsmoeA |
| ASO-583 | 604 | moe(5m)CpsmoeApsmoeGpsmoeApsmoeGpsGpsTpsGypdAypApsGps(5m) |
| | | CpsGpsApsApsmoeGpsmoeTpsmoeGpsmoe(5m)CpsmoeA |
| ASO-584 | 605 | moe(5m)CpsmoeApsmoeGpsmoeApsmoeGpsGpsTpsGpsAypdAypGps(5m) |
| | | CpsGpsApsApsmoeGpsmoeTpsmoeGpsmoe(5m)CpsmoeA |
| ASO-585 | 606 | moe(5m)CpsmoeApsmoeGpsmoeApsmoeGpsGpsTpsGpsApsAypdGyp(5m) |
| | | CpsGpsApsApsmoeGpsmoeTpsmoeGpsmoe(5m)CpsmoeA |
| ASO-586 | 607 | moe(5m)CpsmoeApsmoeGpsmoeApsmoeGpsGpsTpsGpsApsApsGypd(5m) |
| | | CypGpsApsApsmoeGpsmoeTpsmoeGpsmoe(5m)CpsmoeA |
| ASO-587 | 608 | moe(5m)CpsmoeApsmoeGpsmoeApsmoeGpsGpsTpsGpsApsApsGps(5m)Cypd |
| | | GypApsApsmoeGpsmoeTpsmoeGpsmoe(5m)CpsmoeA |
| ASO-588 | 609 | moe(5m)CpsmoeApsmoeGpsmoeApsmoeGpsGpsTpsGpsApsApsGps(5m)Cps |
| | | GypdAypApsmoeGpsmoeTpsmoeGpsmoe(5m)CpsmoeA |
| ASO-589 | 610 | moe(5m)CpsmoeApsmoeGpsmoeApsmoeGpsGpsTpsGpsApsApsGps(5m)Cps |
| | | GpsAypdAypmoeGpsmoeTpsmoeGpsmoe(5m)CpsmoeA |
| ASO-590 | 611 | moeApsmoeGpsmoeGpsmoeTpsmoeGpsApsApsGps(5m)CpsGpsApsApsGps |
| | | TpsGps(5m)CpsmoeApsmoe(5m)CpsmoeApsmoe(5m)CpsmoeG |
| ASO-591 | 612 | moeApsmoeGpsmoeGpsmoeTpsGpsApsApsGps(5m)CpsGpsApsApsGpsTps |
| | | moeGpsmoe(5m)CpsmoeApsmoe(5m)CpsmoeApsmoe(5m)CpsmoeG |
| ASO-592 | 613 | moeApsmoeGpsmoeGpsmoeTpsmoeGpsApsApsGps(5m)CpsGpsApsApsGps |
| | | TpsGpsmoe(5m)CpsmoeApsmoe(5m)CpsmoeApsmoe(5m)CpsmoeG |
| ASO-593 | 614 | moeApsmoeGpsmoeGpsmoeTpsmoeGpsmoeApsApsGps(5m)CpsGpsApsAps |
| | | GpsTpsGps(5m)CpsmoeApsmoe(5m)CpsmoeApsmoe(5m)CpsmoeG |
| ASO-594 | 615 | moeApsmoeGpsmoeGpsmoeTpsmoeGpsmoeApsmoeApsGps(5m)CpsGpsAps |
| | | ApsGpsTpsGps(5m)CpsApsmoe(5m)CpsmoeApsmoe(5m)CpsmoeG |
| ASO-595 | 616 | moeApsmoeGpsmoeGpsmoeTpsGpsApsApsGps(5m)CpsGpsApsApsGpsmoe |
| | | TpsmoeGpsmoe(5m)CpsmoeApsmoe(5m)CpsmoeApsmoe(5m)CpsmoeG |
| ASO-596 | 617 | moeApsmoeGpsmoeGpsmoeTpsmoeGpsApsApsGps(5m)CpsGpsApsApsGps |
| | | TpsmoeGpsmoe(5m)CpsmoeApsmoe(5m)CpsmoeApsmoe(5m)CpsmoeG |
| ASO-597 | 618 | moeApsmoeGpsmoeGpsmoeTpsmoeGpsmoeApsApsGps(5m)CpsGpsApsAps |
| | | GpsTpsGpsmoe(5m)CpsmoeApsmoe(5m)CpsmoeApsmoe(5m)CpsmoeG |
| ASO-598 | 619 | moeApsmoeGpsmoeGpsmoeTpsmoeGpsmoeApsmoeApsGps(5m)CpsGpsAps |
| | | ApsGpsTpsGps(5m)CpsmoeApsmoe(5m)CpsmoeApsmoe(5m)CpsmoeG |
| ASO-599 | 620 | moeApsmoeGpsmoeGpsmoeTpsmoeGpsmoeApsmoeApsmoeGps(5m)CpsGps |
| | | ApsApsGpsTpsGps(5m)CpsApsmoe(5m)CpsmoeApsmoe(5m)CpsmoeG |
| ASO-600 | 621 | moeApsmoeGpsmoeGpsmoeTpsmoeGpsmoeApsApsGps(5m)CpsGpsApsAps |
| | | GpsTpsmoeGpsmoe(5m)CpsmoeApsmoe(5m)CpsmoeApsmoe(5m)CpsmoeG |
| ASO-601 | 622 | moeApsmoeGpsmoeGpsmoeTpsmoeGpsmoeApsmoeApsGps(5m)CpsGpsAps |
| | | ApsGpsTpsGpsmoe(5m)CpsmoeApsmoe(5m)CpsmoeApsmoe(5m)CpsmoeG |
| ASO-602 | 623 | moe(5m)CpsmoeApsmoeGpsmoeApsmoeGpsGpsTypGpsApsApsGps(5m)Cps |
| | | GpsApsApsmoeGpsmoeTpsmoeGpsmoe(5m)CpsmoeA |
| ASO-603 | 624 | moe(5m)CpsmoeApsmoeGpsmoeApsmoeGpsGpsTpsGypApsApsGps(5m)Cps |
| | | GpsApsApsmoeGpsmoeTpsmoeGpsmoe(5m)CpsmoeA |
| ASO-604 | 625 | moe(5m)CpsmoeApsmoeGpsmoeApsmoeGpsGpsTpsGpsAypApsGps(5m)Cps |
| | | GpsApsApsmoeGpsmoeTpsmoeGpsmoe(5m)CpsmoeA |
| ASO-605 | 626 | moe(5m)CpsmoeApsmoeGpsmoeApsmoeGpsGpsTpsGpsApsAypGps(5m)Cps |
| | | GpsApsApsmoeGpsmoeTpsmoeGpsmoe(5m)CpsmoeA |
| ASO-606 | 627 | moe(5m)CpsmoeApsmoeGpsmoeApsmoeGpsGpsTpsGpsApsApsGyp(5m)Cps |
| | | GpsApsApsmoeGpsmoeTpsmoeGpsmoe(5m)CpsmoeA |
| ASO-607 | 628 | moe(5m)CpsmoeApsmoeGpsmoeApsmoeGpsGpsTpsGpsApsApsGps(5m)Cyp |
| | | GpsApsApsmoeGpsmoeTpsmoeGpsmoe(5m)CpsmoeA |
| ASO-608 | 629 | moe(5m)CpsmoeApsmoeGpsmoeApsmoeGpsGpsTpsGpsApsApsGps(5m)Cps |
| | | GypApsApsmoeGpsmoeTpsmoeGpsmoe(5m)CpsmoeA |
| ASO-609 | 630 | moe(5m)CpsmoeApsmoeGpsmoeApsmoeGpsGpsTpsGpsApsApsGps(5m)Cps |
| | | GpsAypApsmoeGpsmoeTpsmoeGpsmoe(5m)CpsmoeA |
| ASO-610 | 631 | moe(5m)CpsmoeApsmoeGpsmoeApsmoeGypGpsTpsGpsApsApsGps(5m)Cps |
| | | GpsApsApsmGpsmoeTpsmGpsmoe(5m)CpsmA |
| ASO-611 | 632 | moe(5m)CpsmoeApsmoeGpsmoeApsmoeGpsGypTpsGpsApsApsGps(5m)Cps |
| | | GpsApsApsmGpsmoeTpsmGpsmoe(5m)CpsmA |
| ASO-612 | 633 | moe(5m)CpsmoeApsmoeGpsmoeApsmoeGpsGpsTypGpsApsApsGps(5m)Cps |
| | | GpsApsApsmGpsmoeTpsmGpsmoe(5m)CpsmA |
| ASO-613 | 634 | moe(5m)CpsmoeApsmoeGpsmoeApsmoeGpsGpsTpsGypApsApsGps(5m)Cps |
| | | GpsApsApsmGpsmoeTpsmGpsmoe(5m)CpsmA |
| ASO-614 | 635 | moe(5m)CpsmoeApsmoeGpsmoeApsmoeGpsGpsTpsGpsAypApsGps(5m)Cps |
| | | GpsApsApsmGpsmoeTpsmGpsmoe(5m)CpsmA |
| ASO-615 | 636 | moe(5m)CpsmoeApsmoeGpsmoeApsmoeGpsGpsTpsGpsApsAypGps(5m)Cps |
| | | GpsApsApsmGpsmoeTpsmGpsmoe(5m)CpsmA |
| ASO-616 | 637 | moe(5m)CpsmoeApsmoeGpsmoeApsmoeGpsGpsTpsGpsApsApsGyp(5m)Cps |
| | | GpsApsApsmGpsmoeTpsmGpsmoe(5m)CpsmA |
| ASO-617 | 638 | moe(5m)CpsmoeApsmoeGpsmoeApsmoeGpsGpsTpsGpsApsApsGps(5m)Cyp |
| | | GpsApsApsmGpsmoeTpsmGpsmoe(5m)CpsmA |
| ASO-618 | 639 | moe(5m)CpsmoeApsmoeGpsmoeApsmoeGpsGpsTpsGpsApsApsGps(5m)Cps |
| | | GypApsApsmGpsmoeTpsmGpsmoe(5m)CpsmA |
| ASO-619 | 640 | moe(5m)CpsmoeApsmoeGpsmoeApsmoeGpsGpsTpsGpsApsApsGps(5m)Cps |
| | | GpsAypApsmGpsmoeTpsmGpsmoe(5m)CpsmA |
| ASO-620 | 641 | moe(5m)CpsmoeApsmoeGpsmoeApsmoeGpsmoeGpsTpsGpsApsApsGps(5m) |
| | | CpsGpsApsApsmoeGpsmoeTpsmoeGpsmoe(5m)CpsmoeA |
| ASO-621 | 642 | moe(5m)CpsmoeApsmoeGpsmoeApsmoeGpsGpsTpsGpsApsApsGps(5m)Cps |
| | | GpsApsmoeApsmoeGpsmoeTpsmoeGpsmoe(5m)CpsmoeA |
| ASO-622 | 643 | moe(5m)CpsmoeApsmoeGpsmoeApsGpsGpsTpsGpsApsApsGps(5m)CpsGps |
| | | moeApsmoeApsmoeGpsmoeTpsmoeGpsmoe(5m)CpsmoeA |
| ASO-623 | 644 | moe(5m)CpsmoeApsmoeGpsmoeApsmoeGpsmoeGpsTpsGpsApsApsGps(5m) |
| | | CpsGpsApsApsGpsmoeTpsmoeGpsmoe(5m)CpsmoeA |
| ASO-624 | 645 | moe(5m)CpsmoeApsmoeGpsmoeApsGpsGpsTpsGpsApsApsGps(5m)CpsGps |
| | | ApsmoeApsmoeGpsmoeTpsmoeGpsmoe(5m)CpsmoeA |
| ASO-625 | 646 | moe(5m)CpsmoeApsmoeGpsmoeApsmoeGpsmoeGpsmoeTpsGpsApsApsGp |
| | | s(5m)CpsGpsApsApsmoeGpsmoeTpsmoeGpsmoe(5m)CpsmoeA |
| ASO-626 | 647 | moe(5m)CpsmoeApsmoeGpsmoeApsmoeGpsmoeGpsTpsGpsApsApsGps |
| | | (5m)CpsGpsApsmoeApsmoeGpsmoeTpsmoeGpsmoe(5m)CpsmoeA |
| ASO-627 | 648 | moe(5m)CpsmoeApsmoeGpsmoeApsmoeGpsGpsTpsGpsApsApsGps(5m)Cps |
| | | GpsmoeApsmoeApsmoeGpsmoeTpsmoeGpsmoe(5m)CpsmoeA |
| ASO-628 | 649 | moe(5m)CpsmoeApsmoeGpsmoeApsmoeGpsmoeGpsmoeTpsGpsApsApsGps |
| | | (5m)CpsGpsApsApsGpsmoeTpsmoeGpsmoe(5m)CpsmoeA |
| ASO-629 | 650 | moeApsmoe(5m)CpsmoeGpsmoeTpsGps(5m)CpsApsGpsApsGpsGpsTpsGps |
| | | ApsmoeApsmoeGpsmoe(5m)CpsmoeGpsmoeApsmoeApsmoeG |
| ASO-630 | 651 | moeApsmoe(5m)CpsmoeGpsmoeTpsmoeGps(5m)CpsApsGpsApsGpsGpsTps |
| | | GpsApsApsmoeGpsmoe(5m)CpsmoeGpsmoeApsmoeApsmoeG |
| ASO-631 | 652 | moeApsmoe(5m)CpsmoeGpsmoeTpsmoeGpsmoe(5m)CpsApsGpsApsGpsGps |
| | | TpsGpsApsApsGpsmoe(5m)CpsmoeGpsmoeApsmoeApsmoeG |
| ASO-632 | 653 | moeApsmoe(5m)CpsmoeGpsmoeTpsmoeGpsmoe(5m)CpsmoeApsGpsApsGps |
| | | GpsTpsGpsApsApsGps(5m)CpsmoeGpsmoeApsmoeApsmoeG |
| ASO-633 | 654 | moeApsmoe(5m)CpsmoeGpsmoeTpsGps(5m)CpsApsGpsApsGpsGpsTpsGps |
| | | moeApsmoeApsmoeGpsmoe(5m)CpsmoeGpsmoeApsmoeApsmoeG |
| ASO-634 | 655 | moeApsmoe(5m)CpsmoeGpsmoeTpsmoeGps(5m)CpsApsGpsApsGpsGpsTps |
| | | GpsApsmoeApsmoeGpsmoe(5m)CpsmoeGpsmoeApsmoeApsmoeG |
| ASO-635 | 656 | moeApsmoe(5m)CpsmoeGpsmoeTpsmoeGpsmoe(5m)CpsApsGpsApsGpsGps |
| | | TpsGpsApsApsmoeGpsmoe(5m)CpsmoeGpsmoeApsmoeApsmoeG |
| ASO-636 | 657 | moeApsmoe(5m)CpsmoeGpsmoeTpsmoeGpsmoe(5m)CpsmoeApsGpsApsGps |
| | | GpsTpsGpsApsApsGpsmoe(5m)CpsmoeGpsmoeApsmoeApsmoeG |
| ASO-637 | 658 | moeApsmoe(5m)CpsmoeGpsmoeTpsmoeGpsmoe(5m)CpsmoeApsmoeGpsAps |
| | | GpsGpsTpsGpsApsApsGps(5m)CpsmoeGpsmoeApsmoeApsmoeG |
| ASO-638 | 659 | moeApsmoe(5m)CpsmoeGpsmoeTpsmoeGpsmoe(5m)CpsApsGpsApsGpsGps |
| | | TpsGpsApsmoeApsmoeGpsmoe(5m)CpsmoeGpsmoeApsmoeApsmoeG |
| ASO-639 | 660 | moeApsmoe(5m)CpsmoeGpsmoeTpsmoeGpsmoe(5m)CpsmoeApsGpsApsGps |
| | | GpsTpsGpsApsApsmoeGpsmoe(5m)CpsmoeGpsmoeApsmoeApsmoeG |
| ASO-640 | 661 | moeApsmoe(5m)CpsmoeGpsmoeTpsmoeGps(5m)CpsApsGpsApsGpsGpsTps |
| | | GpsApsApsGpsmoe(5m)CpsmoeGpsmoeApsmoeApsmoeG |
| ASO-641 | 662 | moe(5m)CpsmoeApslnGpsmoeApsmoeGypGpsTpsGpsApsApsGps(5m)Cps |
| | | GpsApsApsmoeGpsmoeTpslnGpsmoe(5m)CpsmoeA |
| ASO-642 | 663 | moe(5m)CpsmoeApslnGpsmoeApsmoeGpsGypTpsGpsApsApsGps(5m)Cps |
| | | GpsApsApsmoeGpsmoeTpslnGpsmoe(5m)CpsmoeA |
| ASO-643 | 664 | moe(5m)CpsmoeApslnGpsmoeApsmoeGpsGpsTypGpsApsApsGps(5m)Cps |
| | | GpsApsApsmoeGpsmoeTpslnGpsmoe(5m)CpsmoeA |
| ASO-644 | 665 | moe(5m)CpsmoeApslnGpsmoeApsmoeGpsGpsTpsGypApsApsGps(5m)Cps |
| | | GpsApsApsmoeGpsmoeTpslnGpsmoe(5m)CpsmoeA |
| ASO-645 | 666 | moe(5m)CpsmoeApslnGpsmoeApsmoeGpsGpsTpsGpsAypApsGps(5m)Cps |
| | | GpsApsApsmoeGpsmoeTpslnGpsmoe(5m)CpsmoeA |
| ASO-646 | 667 | moe(5m)CpsmoeApslnGpsmoeApsmoeGpsGpsTpsGpsApsAypGps(5m)Cps |
| | | GpsApsApsmoeGpsmoeTpslnGpsmoe(5m)CpsmoeA |
| ASO-647 | 668 | moe(5m)CpsmoeApslnGpsmoeApsmoeGpsGpsTpsGpsApsApsGyp(5m)Cps |
| | | GpsApsApsmoeGpsmoeTpslnGpsmoe(5m)CpsmoeA |
| ASO-648 | 669 | moe(5m)CpsmoeApslnGpsmoeApsmoeGpsGpsTpsGpsApsApsGps(5m)Cyp |
| | | GpsApsApsmoeGpsmoeTpslnGpsmoe(5m)CpsmoeA |
| ASO-649 | 670 | moe(5m)CpsmoeApslnGpsmoeApsmoeGpsGpsTpsGpsApsApsGps(5m)Cps |
| | | GypApsApsmoeGpsmoeTpslnGpsmoe(5m)CpsmoeA |
| ASO-650 | 671 | moe(5m)CpsmoeApslnGpsmoeApsmoeGpsGpsTpsGpsApsApsGps(5m)Cps |
| | | GpsAypApsmoeGpsmoeTpslnGpsmoe(5m)CpsmoeA |
| ASO-651 | 672 | MonoGalNAc4-p-moe(5m)CpsmoeApsmoeGpsmoeApsmoeGpsGpsTpsGps |
| | | ApsApsGps(5m)CpsGpsApsApsmoeGpsmoeTpsmoeGpsmoe(5m)CpsmoeA |
| ASO-652 | 673 | moe(5m)CpsmoeApsmoeGpsmoeApsmoeGpsGpsTpsGpsApsApsGps(5m)Cps |
| | | GpsApsApsmoeGpsmoeTpsmoeGpsmoe(5m)CpsmoeA-monoGalNAc4-p |
| ASO-653 | 674 | diGalNAc4-PS-p-moe(5m)CpsmoeApsmoeGpsmoeApsmoeGpsGpsTpsGps |
| | | ApsApsGps(5m)CpsGpsApsApsmoeGpsmoeTpsmoeGpsmoe(5m)CpsmoeA |
| ASO-654 | 675 | moe(5m)CpsmoeApsmoeGpsmoeApsmoeGpsGpsTpsGpsApsApsGps(5m)Cps |
| | | GpsApsApsmoeGpsmoeTpsmoeGpsmoe(5m)CpsmoeA-diGalNAc4-PS-p |
| ASO-655 | 676 | monoGalNAc4-p-moe(5m)CpsmoeApsmoeGpsmoeApsmoeGpsGpsTpsGps |
| | | ApsApsGps(5m)CpsGpsApsApsmoeGpsmoeTpsmoeGpsmoe(5m)CpsmoeA- |
| | | monGalNAc4-p |
| ASO-656 | 677 | monoGalNAc4-ps-moe(5m)CpsmoeApsmoeGpsmoeApsmoeGpsGpsTpsGps |
| | | ApsApsGps(5m)CpsGpsApsApsmoeGpsmoeTpsmoeGpsmoe(5m)CpsmoeA |
| ASO-657 | 678 | monoGalNAc4-ps-moe(5m)CpsmoeApsmoeGpsmoeApsmoeGpsGpsTpsGps |
| | | ApsApsGps(5m)CpsGpsApsApsmoeGpsmoeTpsmoeGpsmoe(5m)CpsmoeA |
| ASO-658 | 679 | moe(5m)CpsmoeApslnGpsmoeApslnGps5ocpGpsTpsGpsApsApsGps(5m)Cps |
| | | GpsApsApsmoeGpsmoeTpsmoeGpsmoe(5m)CpsmoeA |
| ASO-659 | 680 | moe(5m)CpsmoeApslnGpsmoeApslnGps5omcpGpsTpsGpsApsApsGps(5m) |
| | | CpsGpsApsApsmoeGpsmoeTpsmoeGpsmoe(5m)CpsmoeA |
| ASO-660 | 681 | moe(5m)CpsmoeApslnGpsmoeApslnGpsGpsTps5ocpGpsApsApsGps(5m)Cps |
| | | GpsApsApsmoeGpsmoeTpsmoeGpsmoe(5m)CpsmoeA |
| ASO-661 | 682 | moe(5m)CpsmoeApslnGpsmoeApslnGpsGpsTps5omcGpsApsApsGps(5m)Cps |
| | | GpsApsApsmoeGpsmoeTpsmoeGpsmoe(5m)CpsmoeA |
| ASO-662 | 683 | moe(5m)CpsmoeApslnGpsmoeApsmoeGps5ocpGpsTpsGpsApsApsGps(5m) |
| | | CpsGpsApsApsmoeGpsmoeTpslnGpsmoe(5m)CpsmoeA |
| ASO-663 | 684 | moe(5m)CpsmoeApslnGpsmoeApsmoeGps5omcpGpsTpsGpsApsApsGps(5m) |
| | | CpsGpsApsApsmoeGpsmoeTpslnGpsmoe(5m)CpsmoeA |
| ASO-664 | 685 | moe(5m)CpsmoeApslnGpsmoeApsmoeGpsGpsTps5ocpGpsApsApsGps(5m) |
| | | CpsGpsApsApsmoeGpsmoeTpslnGpsmoe(5m)CpsmoeA |
| ASO-665 | 686 | moe(5m)CpsmoeApslnGpsmoeApsmoeGpsGpsTps5omcpGpsApsApsGps(5m) |
| | | CpsGpsApsApsmoeGpsmoeTpslnGpsmoe(5m)CpsmoeA |
| ASO-666 | 687 | moeApsmoeGpslnGpsmoeTpslnGpsApsAps5ocpGps(5m)CpsGpsApsApsGps |
| | | TpsGpsmoe(5m)CpsmoeApsmoe(5m)CpsmoeApsmoe(5m)C |
| ASO-667 | 688 | moeApsmoeGpslnGpsmoeTpslnGpsApsAps5omcpGps(5m)CpsGpsApsApsGps |
| | | TpsGpsmoe(5m)CpsmoeApsmoe(5m)CpsmoeApsmoe(5m)C |
| ASO-668 | 689 | moe(5m)CpsmoeApslnGpsmoeApsGpsGpsTpsGpsApsApsGps(5m)CpsGpsAps |
| | | ApsmoeGpsmoeTpslnGpsmoe(5m)CpsmoeA |
| ASO-669 | 690 | moe(5m)CpsmoeApslnGpsmoeApsmoeGpsGpsTpsGpsApsApsGps(5m)Cps |
| | | GpsApsApsGpsmoeTpslnGpsmoe(5m)CpsmoeA |
| ASO-670 | 691 | moe(5m)CpsmoeApslnGpsmoeApsmoeGpsmoeGpsTpsGpsApsApsGps(5m) |
| | | CpsGpsApsApsmoeGpsmoeTpslnGpsmoe(5m)CpsmoeA |
| ASO-671 | 692 | moe(5m)CpsmoeApslnGpsmoeApsmoeGpsGpsTpsGpsApsApsGps(5m)Cps |
| | | GpsApsmoeApsmoeGpsmoeTpslnGpsmoe(5m)CpsmoeA |
| ASO-672 | 693 | moeGpsmoe(5m)CpsmoeApsmoeGpsmoeApsmXpsGpsTpsGpsApsApsGps |
| | | (5m)CpsGpsApsmoeApsmoeGpsmoeTpsmoeGpsmoe(5m)C |
| ASO-673 | 694 | moeGpsmoe(5m)CpsmoeApsmoeGpsmoeApsGpsmXpsTpsGpsApsApsGps |
| | | (5m)CpsGpsApsmoeApsmoeGpsmoeTpsmoeGpsmoe(5m)C |
| ASO-674 | 695 | moeGpsmoe(5m)CpsmoeApsmoeGpsmoeApsGpsGpsmXpsGpsApsApsGps |
| | | (5m)CpsGpsApsmoeApsmoeGpsmoeTpsmoeGpsmoe(5m)C |
| ASO-675 | 696 | moeGpsmoe(5m)CpsmoeApsmoeGpsmoeApsGpsGpsTpsmXpsApsApsGps |
| | | (5m)CpsGpsApsmoeApsmoeGpsmoeTpsmoeGpsmoe(5m)C |
| ASO-676 | 697 | moeGpsmoe(5m)CpsmoeApsmoeGpsmoeApsGpsGpsTpsGpsmXpsApsGps |
| | | (5m)CpsGpsApsmoeApsmoeGpsmoeTpsmoeGpsmoe(5m)C |
| ASO-677 | 698 | moeGpsmoe(5m)CpsmoeApsmoeGpsmoeApsGpsGpsTpsGpsApsmXpsGps |
| | | (5m)CpsGpsApsmoeApsmoeGpsmoeTpsmoeGpsmoe(5m)C |
| ASO-678 | 699 | moeGpsmoe(5m)CpsmoeApsmoeGpsmoeApsGpsGpsTpsGpsApsApsmXps |
| | | (5m)CpsGpsApsmoeApsmoeGpsmoeTpsmoeGpsmoe(5m)C |
| ASO-679 | 700 | moeGpsmoe(5m)CpsmoeApsmoeGpsmoeApsGpsGpsTpsGpsApsApsGpsmXps |
| | | GpsApsmoeApsmoeGpsmoeTpsmoeGpsmoe(5m)C |
| ASO-680 | 701 | moeGpsmoe(5m)CpsmoeApsmoeGpsmoeApsGpsGpsTpsGpsApsApsGps(5m) |
| | | CpsmXpsApsmoeApsmoeGpsmoeTpsmoeGpsmoe(5m)C |
| ASO-681 | 702 | moeGpsmoe(5m)CpsmoeApsmoeGpsmoeApsGpsGpsTpsGpsApsApsGps(5m) |
| | | CpsGpsmXpsmoeApsmoeGpsmoeTpsmoeGpsmoe(5m)C |
| ASO-682 | 703 | moeGpsmoe(5m)CpsmoeApsmoeGpsmoeApsGpsGpsTpsApsApsApsGps(5m) |
| | | CpsGpsApsmoeApsmoeGpsmoeTpsmoeGpsmoe(5m)C |
| ASO-683 | 704 | moeGpsmoe(5m)CpsmoeApsmoeGpsmoeApsGpsGpsTps(5m)CpsApsApsGps |
| | | (5m)CpsGpsApsmoeApsmoeGpsmoeTpsmoeGpsmoe(5m)C |
| ASO-684 | 705 | moeGpsmoe(5m)CpsmoeApsmoeGpsmoeApsGpsGpsTpsTpsApsApsGps(5m) |
| | | CpsGpsApsmoeApsmoeGpsmoeTpsmoeGpsmoe(5m)C |
| ASO-685 | 706 | moeGpsmoe(5m)CpsmoeApsmoeGpsmoeApsGpsGpsTpsGps(5m)CpsApsGps |
| | | (5m)CpsGpsApsmoeApsmoeGpsmoeTpsmoeGpsmoe(5m)C |
| ASO-686 | 707 | moeGpsmoe(5m)CpsmoeApsmoeGpsmoeApsGpsGpsTpsGpsGpsApsGps(5m) |
| | | CpsGpsApsmoeApsmoeGpsmoeTpsmoeGpsmoe(5m)C |
| ASO-687 | 708 | moeGpsmoe(5m)CpsmoeApsmoeGpsmoeApsGpsGpsTpsGpsTpsApsGps(5m) |
| | | CpsGpsApsmoeApsmoeGpsmoeTpsmoeGpsmoe(5m)C |
| ASO-688 | 709 | moeGpsmoe(5m)CpsmoeApsmoeGpsmoeApsGpsGpsTpsGpsAps(5m)CpsGps |
| | | (5m)CpsGpsApsmoeApsmoeGpsmoeTpsmoeGpsmoe(5m)C |
| ASO-689 | 710 | moeGpsmoe(5m)CpsmoeApsmoeGpsmoeApsGpsGpsTpsGpsApsGpsGps(5m) |
| | | CpsGpsApsmoeApsmoeGpsmoeTpsmoeGpsmoe(5m)C |
| ASO-690 | 711 | moeGpsmoe(5m)CpsmoeApsmoeGpsmoeApsGpsGpsTpsGpsApsTpsGps(5m) |
| | | CpsGpsApsmoeApsmoeGpsmoeTpsmoeGpsmoe(5m)C |
| ASO-691 | 712 | moeGpsmoe(5m)CpsmoeApsmoeGpsmoeApsGpsGpsTpsGpsApsApsAps(5m) |
| | | CpsGpsApsmoeApsmoeGpsmoeTpsmoeGpsmoe(5m)C |
| ASO-692 | 713 | moeGpsmoe(5m)CpsmoeApsmoeGpsmoeApsGpsGpsTpsGpsApsAps(5m)Cps |
| | | (5m)CpsGpsApsmoeApsmoeGpsmoeTpsmoeGpsmoe(5m)C |
| ASO-693 | 714 | moeGpsmoe(5m)CpsmoeApsmoeGpsmoeApsGpsGpsTpsGpsApsApsTps(5m) |
| | | CpsGpsApsmoeApsmoeGpsmoeTpsmoeGpsmoe(5m)C |
| ASO-694 | 715 | moe(5m)CpslnApsmoeGpsmoeApsmoeGpsGpsTpsGpsApsApsGps(5m)Cps |
| | | GpsApsApsmoeGpslnTpsmoeGpsr?moe(5m)CpsmoeA |
| ASO-695 | 716 | moe(5m)CpsmoeApslnGpsmoeApsmoeGpsGpsTpsGpsApsApsGps(5m)Cps |
| | | GpsApsApsmoeGpslnTpsmoeGpsr?moe(5m)CpsmoeA |
| ASO-696 | 717 | moeGpsmoe(5m)CpsmoeApsmoeGpsmoeApsGpsGpsTpsGpsmoeApsApsGps |
| | | (5m)CpsGpsApsmoeApslnGpsmoeTpslnGpsmoe(5m)C |
| ASO-697 | 718 | moeGpsmoe(5m)CpsmoeApsmoeGpsmoeApsGpsGpsTpsGpsmoeApsApsGps |
| | | (5m)CpsGpsApsmoeApslnGpsmoeTpsmoeGpsln(5m)C |
| ASO-698 | 719 | moeGpsmoe(5m)CpsmoeApsmoeGpsmoeApsGpsGpsTpsGpsmoeApsApsGps |
| | | (5m)CpsGpsApsmoeApsmoeGpsmoeTpslnGpsln(5m)C |
| ASO-699 | 720 | moeGpsmoe(5m)CpsmoeApsmoeGpsmoeApsGpsGpsTpsGpsmoeApsApsGps |
| | | (5m)CpsGpsApsmoeApslnGpsmoesmoeGpsmoe(5m)C |
| ASO-700 | 721 | moeGpsmoe(5m)CpsmoeApsmoeGpsmoeApsGpsGpsTpsGpsmoeApsApsGps |
| | | (5m)CpsGpsApsmoeApsmoeGpsmoeTpslnGpsmoe(5m)C |
| ASO-701 | 722 | moeGpsmoe(5m)CpsmoeApsmoeGpsmoeApsGpsGpsTpsGpsmoeApsApsGps |
| | | (5m)CpsGpsApsmoeApsmoeGpsmoeTpsmoeGpsln(5m)C |
| ASO-702 | 723 | moeGpsmoe(5m)CpsmoeApsmoeGpsmoeApsdXpsGpsTpsGpsApsApsGps(5m) |
| | | CpsGpsApsmoeApsmoeGpsmoeTpsmoeGpsmoe(5m)C |
| ASO-703 | 724 | moeGpsmoe(5m)CpsmoeApsmoeGpsmoeApsGpsdXpsTpsGpsApsApsGps(5m) |
| | | CpsGpsApsmoeApsmoeGpsmoeTpsmoeGpsmoe(5m)C |
| ASO-704 | 725 | moeGpsmoe(5m)CpsmoeApsmoeGpsmoeApsGpsGpsdXpsGpsApsApsGps(5m) |
| | | CpsGpsApsmoeApsmoeGpsmoeTpsmoeGpsmoe(5m)C |
| ASO-705 | 726 | moeGpsmoe(5m)CpsmoeApsmoeGpsmoeApsGpsGpsTpsdXpsApsApsGps(5m) |
| | | CpsGpsApsmoeApsmoeGpsmoeTpsmoeGpsmoe(5m)C |
| ASO-706 | 727 | moeGpsmoe(5m)CpsmoeApsmoeGpsmoeApsGpsGpsTpsGpsdXpsApsGps(5m) |
| | | CpsGpsApsmoeApsmoeGpsmoeTpsmoeGpsmoe(5m)C |
| ASO-707 | 728 | moeGpsmoe(5m)CpsmoeApsmoeGpsmoeApsGpsGpsTpsGpsApsdXpsGps(5m) |
| | | CpsGpsApsmoeApsmoeGpsmoeTpsmoeGpsmoe(5m)C |
| ASO-708 | 729 | moeGpsmoe(5m)CpsmoeApsmoeGpsmoeApsGpsGpsTpsGpsApsApsdXps(5m) |
| | | CpsGpsApsmoeApsmoeGpsmoeTpsmoeGpsmoe(5m)C |
| ASO-709 | 730 | moeGpsmoe(5m)CpsmoeApsmoeGpsmoeApsGpsGpsTpsGpsApsApsGpsdXps |
| | | GpsApsmoeApsmoeGpsmoeTpsmoeGpsmoe(5m)C |
| ASO-710 | 731 | moeGpsmoe(5m)CpsmoeApsmoeGpsmoeApsGpsGpsTpsGpsApsApsGps(5m) |
| | | CpsdXpsApsmoeApsmoeGpsmoeTpsmoeGpsmoe(5m)C |
| ASO-711 | 732 | moeGpsmoe(5m)CpsmoeApsmoeGpsmoeApsGpsGpsTpsGpsApsApsGps(5m) |
| | | CpsGpsdXpsmoeApsmoeGpsmoeTpsmoeGpsmoe(5m)C |
| ASO-712 | 733 | moe(5m)CpsmoeApsmoeGpsmoeApsmoeGpsGpsTpsGpsApsApsGps(5m)Cps |
| | | GpsApsApsmoeGpsgutbTpsmoeGpsmoe(5m)CpsmoeA |
| ASO-713 | 734 | moe(5m)CpsmoeApsmoeGpsmoeApsmoeGpsGpsTpsGpsApsApsGps(5m)Cps |
| | | GpsApsApsmoeGpsmoeTpsgutbGpsmoe(5m)CpsmoeA |
| ASO-714 | 735 | moe(5m)CpsmoeApsmoeGpsmoeApsmoeGpsGpsTpsGpsApsApsGps(5m)Cps |
| | | GpsApsApsmoeGpsmoeTpsmoeGpsgutb(5m)CpsmoeA |
| ASO-715 | 736 | moe(5m)CpsgutbApsmoeGpsmoeApsmoeGpsGpsTpsGpsApsApsGps(5m)Cps |
| | | GpsApsApsmoeGpsmoeTpsmoeGpsgutb(5m)CpsmoeA |
| ASO-716 | 737 | moeGpsgutb(5m)CpsmoeApsmoeGpsApsGpsGpsTpsGpsmXpsApsGps(5m) |
| | | CpsGpsApsmoeApsmoeGpsmoeTpsmoeGpsmoe(5m)C |
| ASO-717 | 738 | moeGpsmoe(5m)CpsgutbApsmoeGpsApsGpsGpsTpsGpsmXpsApsGps(5m) |
| | | CpsGpsApsmoeApsmoeGpsmoeTpsmoeGpsmoe(5m)C |
| ASO-718 | 739 | moeGpsmoe(5m)CpsmoeApsmoeGpsApsGpsGpsTpsGpsmXpsApsGps(5m) |
| | | CpsGpsApsgutbApsmoeGpsmoeTpsmoeGpsmoe(5m)C |
| ASO-719 | 740 | moeGpsmoe(5m)CpsmoeApsmoeGpsApsGpsGpsTpsGpsmXpsApsGps(5m) |
| | | CpsGpsApsmoeApsmoeGpsgutbTpsmoeGpsmoe(5m)C |
| ASO-720 | 741 | moeGpsmoe(5m)CpsmoeApsmoeGpsApsGpsGpsTpsGpsmXpsApsGps(5m) |
| | | CpsGpsApsmoeApsmoeGpsmoeTpsmoeGpsgutb(5m)C |
| ASO-721 | 742 | moeGpsgutb(5m)CpsgutbApsmoeGpsApsGpsGpsTpsGpsmXpsApsGps(5m) |
| | | CpsGpsApsmoeApsmoeGpsmoeTpsmoeGpsmoe(5m)C |
| ASO-722 | 743 | moeGpsgutb(5m)CpsmoeApsmoeGpsApsGpsGpsTpsGpsmXpsApsGps(5m) |
| | | CpsGpsApsgutbApsmoeGpsmoeTpsmoeGpsmoe(5m)C |
| ASO-723 | 744 | moeGpsgutb(5m)CpsmoeApsmoeGpsApsGpsGpsTpsGpsmXpsApsGps(5m) |
| | | CpsGpsApsmoeApsmoeGpsgutbTpsmoeGpsmoe(5m)C |
| ASO-724 | 745 | moeGpsgutb(5m)CpsmoeApsmoeGpsApsGpsGpsTpsGpsmXpsApsGps(5m) |
| | | CpsGpsApsmoeApsmoeGpsmoeTpsmoeGpsgutb(5m)C |
| ASO-725 | 746 | moeGpsmoe(5m)CpsgutbApsmoeGpsApsGpsGpsTpsGpsmXpsApsGps(5m) |
| | | CpsGpsApsgutbApsmoeGpsmoeTpsmoeGpsmoe(5m)C |
| ASO-726 | 747 | moeGpsmoe(5m)CpsgutbApsmoeGpsApsGpsGpsTpsGpsmXpsApsGps(5m) |
| | | CpsGpsApsmoeApsmoeGpsgutbTpsmoeGpsmoe(5m)C |
| ASO-727 | 748 | moeGpsmoe(5m)CpsgutbApsmoeGpsApsGpsGpsTpsGpsmXpsApsGps(5m) |
| | | CpsGpsApsmoeApsmoeGpsmoeTpsmoeGpsgutb(5m)C |
| ASO-728 | 749 | moeGpsmoe(5m)CpsmoeApsmoeGpsApsGpsGpsTpsGpsmXpsApsGps(5m) |
| | | CpsGpsApsgutbApsmoeGpsgutbTpsmoeGpsmoe(5m)C |
| ASO-729 | 750 | moeGpsmoe(5m)CpsmoeApsmoeGpsApsGpsGpsTpsGpsmXpsApsGps(5m) |
| | | CpsGpsApsgutbApsmoeGpsmoeTpsmoeGpsgutb(5m)C |
| ASO-730 | 751 | moeGpsmoe(5m)CpsmoeApsmoeGpsApsGpsGpsTpsGpsmXpsApsGps(5m) |
| | | CpsGpsApsmoeApsmoeGpsgutbTpsmoeGpsgutb(5m)C |
| ASO-731 | 752 | moeTpsmoeGpsmoe(5m)CpsmoeApsmoeGpsApsGpsGpsTpsGpsApsApsGps |
| | | (5m)CpsGpsmoeApsmoeApsmoeGpsmoeTpsmoeGpsmoe(5m)C |
| ASO-732 | 753 | moeTpsmoeGpsmoe(5m)CpsmoeApsmoeGpsmoeApsGpsGpsTpsGpsApsAps |
| | | Gps(5m)CpsGpsApsmoeApsmoeGpsmoeTpsmoeGpsmoe(5m)C |
| ASO-733 | 754 | moeGpsmoeTpsmoeGpsmoe(5m)CpsmoeApsGpsApsGpsGpsTpsGpsApsAps |
| | | Gps(5m)CpsmoeGpsmoeApsmoeApsmoeGpsmoeTpsmoeG |
| ASO-734 | 755 | moeGpsmoeTpsmoeGpsmoe(5m)CpsmoeApsmoeGpsApsGpsGpsTpsGpsAps |
| | | ApsGps(5m)CpsGpsmoeApsmoeApsmoeGpsmoeTpsmoeG |
| ASO-735 | 756 | moe(5m)CpsmoeGpsmoeTpsmoeGpsmoe(5m)CpsApsGpsApsGpsGpsTpsGps |
| | | ApsApsGpsmoe(5m)CpsmoeGpsmoeApsmoeApsmoeGpsmoeT |
| ASO-736 | 757 | moe(5m)CpsmoeGpsmoeTpsmoeGpsmoe(5m)CpsmoeApsGpsApsGpsGpsTps |
| | | GpsApsApsGps(5m)CpsmoeGpsmoeApsmoeApsmoeGpsmoeT |
| ASO-737 | 758 | moe(5m)CpslnApsmoeGpsmoeApsmoeGpsGpsTpsGpsApsApsGps(5m)Cps |
| | | GpsApsApsmGpsmoeTpslnGpsmoe(5m)CpsmA |
| ASO-738 | 759 | moe(5m)CpslnApsmoeGpsmoeApsmoeGpsGpsTpsGpsApsApsGps(5m)Cps |
| | | GpsApsApsmoeGpsmoeTpslnGpsm(5m)CpsmA |
| ASO-739 | 760 | moe(5m)CpslnApsmoeGpsmoeApsmoeGpsGpsTpsGpsApsApsGps(5m)Cps |
| | | GpsApsApsmGpslnTpsmGpsmoe(5m)CpsmoeA |
| ASO-740 | 761 | moe(5m)CpslnApsmoeGpsmoeApsmoeGpsGpsTpsGpsApsApsGps(5m)Cps |
| | | GpsApsApsmoeGpslnTpsmGpsm(5m)CpsmoeA |
| ASO-741 | 762 | moe(5m)CpsmoeApslnGpsmoeApsmoeGpsGpsTpsGpsApsApsGps(5m)Cps |
| | | GpsApsApsmGpsmoeTpsmGpsln(5m)CpsmoeA |
| ASO-742 | 763 | moe(5m)CpsmoeApslnGpsmoeApsmoeGpsGpsTpsGpsApsApsGps(5m)Cps |
| | | GpsApsApsmoeGpsmoeTpsmGpsln(5m)CpsmA |
| ASO-743 | 764 | moeGpsmoe(5m)CpsmoeApsmoeGpsmoeApsApsGpsTpsGpsApsApsGps(5m) |
| | | CpsGpsApsmoeApsmoeGpsmoeTpsmoeGpsmoe(5m)C |
| ASO-744 | 765 | moeGpsmoe(5m)CpsmoeApsmoeGpsmoeAps(5m)CpsGpsTpsGpsApsApsGps |
| | | (5m)CpsGpsApsmoeApsmoeGpsmoeTpsmoeGpsmoe(5m)C |
| ASO-745 | 766 | moeGpsmoe(5m)CpsmoeApsmoeGpsmoeApsTpsGpsTpsGpsApsApsGps(5m) |
| | | CpsGpsApsmoeApsmoeGpsmoeTpsmoeGpsmoe(5m)C |
| ASO-746 | 767 | moeGpsmoe(5m)CpsmoeApsmoeGpsmoeApsGpsApsTpsGpsApsApsGps(5m) |
| | | CpsGpsApsmoeApsmoeGpsmoeTpsmoeGpsmoe(5m)C |
| ASO-747 | 768 | moeGpsmoe(5m)CpsmoeApsmoeGpsmoeApsGps(5m)CpsTpsGpsApsApsGps |
| | | (5m)CpsGpsApsmoeApsmoeGpsmoeTpsmoeGpsmoe(5m)C |
| ASO-748 | 769 | moeGpsmoe(5m)CpsmoeApsmoeGpsmoeApsGpsTpsTpsGpsApsApsGps(5m) |
| | | CpsGpsApsmoeApsmoeGpsmoeTpsmoeGpsmoe(5m)C |
| ASO-749 | 770 | moeGpsmoe(5m)CpsmoeApsmoeGpsmoeApsGpsGpsApsGpsApsApsGps(5m) |
| | | CpsGpsApsmoeApsmoeGpsmoeTpsmoeGpsmoe(5m)C |
| ASO-750 | 771 | moeGpsmoe(5m)CpsmoeApsmoeGpsmoeApsGpsGps(5m)CpsGpsApsApsGps |
| | | (5m)CpsGpsApsmoeApsmoeGpsmoeTpsmoeGpsmoe(5m)C |
| ASO-751 | 772 | moeGpsmoe(5m)CpsmoeApsmoeGpsmoeApsGpsGpsGpsGpsApsApsGps(5m) |
| | | CpsGpsApsmoeApsmoeGpsmoeTpsmoeGpsmoe(5m)C |
| ASO-752 | 773 | moe(5m)CpsmoeApsmoeGpsmoeApsmoeGpsGpsTpsGpsApsApsd(8nh)Gps |
| | | (5m)CpsGpsApsApslnGpsmoeTpslnGpsmoe(5m)CpsgutbA |
| ASO-753 | 774 | moeGpsmoe(5m)CpsmoeApsmoeGpsmoeApsGpsGpsTpsGpsApsApsGps(5m) |
| | | CpsGpsGpsmoeApsmoeGpsmoeTpsmoeTpsmoe(5m)C |
| ASO-754 | 775 | moeGpsmoe(5m)CpsmoeApsmoeGpsApsGpsGpsTpsGpsmoeApsApsGps(5m) |
| | | CpsGpsApsmoeApslnApsmoeTpslnGpsln(5m)C |
| ASO-755 | 776 | moeGpsmoe(5m)CpsmoeApsmoeGpsmoeApsGpsGpsTpsGpsApsApsGps(5m) |
| | | CpsGpsTpsmoeApsmoeGpsmoeTpsmoeTpsmoe(5m)C |
| ASO-756 | 777 | moeGpsmoe(5m)CpsmoeApsmoeGpsmoeApsGpsGpsTpsGpsApsApsGps(5m) |
| | | CpsGps(5m)CpsmoeApsmoeGpsmoeTpsmoeTpsmoe(5m)C |
| ASO-757 | 778 | moeGpsmoe(5m)CpsmoeApsmoeGpsmoeApsGpsGpsTpsGpsApsApsGps(5m) |
| | | CpsApsApsmoeApsmoeGpsmoeTpsmoeTpsmoe(5m)C |
| ASO-758 | 779 | moeGpsmoe(5m)CpsmoeApsmoeGpsmoeApsGpsGpsTpsGpsApsApsGps(5m) |
| | | CpsTpsApsmoeApsmoeGpsmoeTpsmoeTpsmoe(5m)C |
| ASO-759 | 780 | moeGpsmoe(5m)CpsmoeApsmoeGpsmoeApsGpsGpsTpsGpsApsApsGps(5m) |
| | | Cps(5m)CpsApsmoeApsmoeGpsmoeTpsmoeTpsmoe(5m)C |
| ASO-760 | 781 | moeGpsmoe(5m)CpsmoeApsmoeGpsmoeApsGpsGpsTpsGpsApsApsGpsAps |
| | | GpsApsmoeApsmoeGpsmoeTpsmoeTpsmoe(5m)C |
| ASO-761 | 782 | moeGpsmoe(5m)CpsmoeApsmoeGpsmoeApsGpsGpsTpsGpsApsApsGpsTps |
| | | GpsApsmoeApsmoeGpsmoeTpsmoeTpsmoe(5m)C |
| ASO-762 | 783 | moeGpsmoe(5m)CpsmoeApsmoeGpsmoeApsGpsGpsTpsGpsApsApsGpsGps |
| | | GpsApsmoeApsmoeGpsmoeTpsmoeTpsmoe(5m)C |
| ASO-763 | 784 | moeGpsmoe(5m)CpsmoeApsmoeGpsmoeApsGpsGpsTpsGpsApsApsGps(5m) |
| | | CpsGpsApsmXpsmoeGpsmoeTpsmoeTpsmoe(5m)C |
| ASO-764 | 785 | moeGpsmoe(5m)CpsmoeApsmoeGpsmoeApsGpsGpsTpsGpsApsApsGps(5m) |
| | | CpsGpsApsmoeApsmXpsmoeTpsmoeTpsmoe(5m)C |
| ASO-765 | 786 | moeGpsmoe(5m)CpsmoeApsmoeGpsmoeApsGpsGpsTpsGpsApsApsGps(5m) |
| | | CpsGpsApsmoeApsmoeGpsmXpsmoeTpsmoe(5m)C |
| ASO-766 | 787 | moeGpsmoe(5m)CpsmoeApsmoeGpsmoeApsGpsGpsTpsGpsApsApsGps(5m) |
| | | CpsGpsApsmoeApsmoeGpsmoeTpsmXpsmoe(5m)C |
| ASO-767 | 788 | moeGpsmoe(5m)CpsmoeApsmoeGpsmoeApsGpsGpsTpsGpsApsApsGps(5m) |
| | | CpsGpsApsmoeApsmoeGpsmoeTpsmoeTpsmX |
| ASO-768 | 789 | moeGpsmoe(5m)CpsmoeApsmoeGpsApsGpsGpsTpsGpsmoeApsApsGps(5m) |
| | | CpsGpsApsmoeApsln(5m)CpsmoeTpslnGpsln(5m)C |
| ASO-769 | 790 | moeGpsmoe(5m)CpsmoeApsmoeGpsApsGpsGpsTpsGpsmoeApsApsGps(5m) |
| | | CpsGpsApsmoeApslnTpsmoeTpslnGpsln(5m)C |
| ASO-770 | 791 | moeGpsmoe(5m)CpsmoeApsmoeGpsApsGpsGpsTpsGpsmoeApsApsGps(5m) |
| | | CpsGpsApsmoeApslnGpsmoeTpslnApsln(5m)C |
| ASO-771 | 792 | moeGpsmoe(5m)CpsmoeApsmoeGpsApsGpsGpsTpsGpsmoeApsApsGps(5m) |
| | | CpsGpsApsmoeApslnGpsmoeTpsln(5m)Cpsln(5m)C |
| ASO-772 | 793 | moeGpsmoe(5m)CpsmoeApsmoeGpsApsGpsGpsTpsGpsmoeApsApsGps(5m) |
| | | CpsGpsApsmoeApslnGpsmoeTpslnTpsln(5m)C |
| ASO-773 | 794 | moeGpsmoe(5m)CpsmoeApsmoeGpsApsGpsGpsTpsGpsmoeApsApsGps(5m) |
| | | CpsGpsApsmoeApslnGpsmoeTpslnGpslnA |
| ASO-774 | 795 | moeGpsmoe(5m)CpsmoeApsmoeGpsApsGpsGpsTpsGpsmoeApsApsGps(5m) |
| | | CpsGpsApsmoeApslnGpsmoeTpslnGpslnG |
| ASO-775 | 796 | moeGpsmoe(5m)CpsmoeApsmoeGpsApsGpsGpsTpsGpsmoeApsApsGps(5m) |
| | | CpsGpsApsmoeApslnGpsmoeTpslnGpslnT |
| ASO-776 | 797 | moeGpsmoeGpsmoeTpsmoeGpsmoeApsApsGps(5m)CpsGpsApsApsGpsTps |
| | | Gps(5m)CpsmoeApsmoe(5m)CpsmoeApsmoe(5m)CpsmoeGpsmoeG |
| ASO-777 | 798 | moeGpsmoeGpsmoeTpsmoeGpsmoeApsmoeApsGps(5m)CpsGpsApsApsGps |
| | | TpsGps(5m)CpsApsmoe(5m)CpsmoeApsmoe(5m)CpsmoeGpsmoeG |
| ASO-778 | 799 | moeGpsmoeApsmoeGpsmoeGpsmoeTpsGpsApsApsGps(5m)CpsGpsApsAps |
| | | GpsTpsmoeGpsmoe(5m)CpsmoeApsmoe(5m)CpsmoeApsmoe(5m)C |
| ASO-779 | 800 | moeGpsmoeApsmoeGpsmoeGpsmoeTpsmoeGpsApsApsGps(5m)CpsGpsAps |
| | | ApsGpsTpsGpsmoe(5m)CpsmoeApsmoe(5m)CpsmoeApsmoe(5m)C |
| ASO-780 | 801 | moeApsmoeGpsmoeApsmoeGpsmoeGpsTpsGpsApsApsGps(5m)CpsGpsAps |
| | | ApsGpsmoeTpsmoeGpsmoe(5m)CpsmoeApsmoe(5m)CpsmoeA |
| ASO-781 | 802 | moeApsmoeGpsmoeApsmoeGpsmoeGpsmoeTpsGpsApsApsGps(5m)CpsGps |
| | | ApsApsGpsTpsmoeGpsmoe(5m)CpsmoeApsmoe(5m)CpsmoeA |
| ASO-782 | 803 | moeGpsmoe(5m)CpsmoeApsmoeGpsmoeApsGpsGpsTpsGpsApsApsGps(5m) |
| | | CpsGpsApsmoeApsmoeGpsmoeTpsmoeGpsmoe(5m)CpsmoeA |
| ASO-783 | 804 | moeGpsmoe(5m)CpsmoeApsmoeGpsmoeApsmoeGpsGpsTpsGpsApsApsGps |
| | | (5m)CpsGpsApsApsmoeGpsmoeTpsmoeGpsmoe(5m)CpsmoeA |
| ASO-784 | 805 | moe(5m)CpsmoeApsmoeGpsmoeApsmoeGpsGpsTpsGpsApsApsd(8nh)Gps |
| | | (5m)CpsGpsApsApsgutbGpsmoeTpslnGpsmoe(5m)CpslnA |
| ASO-785 | 806 | moe(5m)CpsmoeApsmoeGpsmoeApsmoeGpsGpsTpsGpsApsApsd(8nh)Gps |
| | | (5m)CpsGpsApsApslnGpsmoeTpsgutbGpsmoe(5m)CpslnA |
| ASO-786 | 807 | lnGpsln(5m)CpsmoeApsmoeGpsmoeApsmXpsGpsTpsGpsApsApsGps(5m)Cps |
| | | GpsApsmoeApsmoeGpsmoeTpsmoeGpsmoe(5m)C |
| ASO-787 | 808 | lnGpsmoe(5m)CpslnApsmoeGpsmoeApsmXpsGpsTpsGpsApsApsGps(5m)Cps |
| | | GpsApsmoeApsmoeGpsmoeTpsmoeGpsmoe(5m)C |
| ASO-788 | 809 | lnGpsmoe(5m)CpsmoeApslnGpsmoeApsmXpsGpsTpsGpsApsApsGps(5m)Cps |
| | | GpsApsmoeApsmoeGpsmoeTpsmoeGpsmoe(5m)C |
| ASO-789 | 810 | lnGpsmoe(5m)CpsmoeApsmoeGpslnApsmXpsGpsTpsGpsApsApsGps(5m)Cps |
| | | GpsApsmoeApsmoeGpsmoeTpsmoeGpsmoe(5m)C |
| ASO-790 | 811 | lnGpsmoe(5m)CpsmoeApsmoeGpsmoeApsmXpsGpsTpsGpsApsApsGps(5m) |
| | | CpsGpsApslnApsmoeGpsmoeTpsmoeGpsmoe(5m)C |
| ASO-791 | 812 | lnGpsmoe(5m)CpsmoeApsmoeGpsmoeApsmXpsGpsTpsGpsApsApsGps(5m) |
| | | CpsGpsApsmoeApslnGpsmoeTpsmoeGpsmoe(5m)C |
| ASO-792 | 813 | lnGpsmoe(5m)CpsmoeApsmoeGpsmoeApsmXpsGpsTpsGpsApsApsGps(5m) |
| | | CpsGpsApsmoeApsmoeGpslnTpsmoeGpsmoe(5m)C |
| ASO-793 | 814 | lnGpsmoe(5m)CpsmoeApsmoeGpsmoeApsmXpsGpsTpsGpsApsApsGps(5m) |
| | | CpsGpsApsmoeApsmoeGpsmoeTpslnGpsmoe(5m)C |
| ASO-794 | 815 | lnGpsmoe(5m)CpsmoeApsmoeGpsmoeApsmXpsGpsTpsGpsApsApsGps(5m) |
| | | CpsGpsApsmoeApsmoeGpsmoeTpsmoeGpsln(5m)C |
| ASO-795 | 816 | moeGpsln(5m)CpslnApsmoeGpsmoeApsmXpsGpsTpsGpsApsApsGps(5m) |
| | | CpsGpsApsmoeApsmoeGpsmoeTpsmoeGpsmoe(5m)C |
| ASO-796 | 817 | moeGpsln(5m)CpsmoeApslnGpsmoeApsmXpsGpsTpsGpsApsApsGps(5m) |
| | | CpsGpsApsmoeApsmoeGpsmoeTpsmoeGpsmoe(5m)C |
| ASO-797 | 818 | moeGpsln(5m)CpsmoeApsmoeGpslnApsmXpsGpsTpsGpsApsApsGps(5m) |
| | | CpsGpsApsmoeApsmoeGpsmoeTpsmoeGpsmoe(5m)C |
| ASO-798 | 819 | moeGpsln(5m)CpsmoeApsmoeGpsmoeApsmXpsGpsTpsGpsApsApsGps(5m) |
| | | CpsGpsApslnApsmoeGpsmoeTpsmoeGpsmoe(5m)C |
| ASO-799 | 820 | moeGpsln(5m)CpsmoeApsmoeGpsmoeApsmXpsGpsTpsGpsApsApsGps(5m) |
| | | CpsGpsApsmoeApslnGpsmoeTpsmoeGpsmoe(5m)C |
| ASO-800 | 821 | moeGpsln(5m)CpsmoeApsmoeGpsmoeApsmXpsGpsTpsGpsApsApsGps(5m) |
| | | CpsGpsApsmoeApsmoeGpslnTpsmoeGpsmoe(5m)C |
| ASO-801 | 822 | moeGpsln(5m)CpsmoeApsmoeGpsmoeApsmXpsGpsTpsGpsApsApsGps(5m) |
| | | CpsGpsApsmoeApsmoeGpsmoeTpslnGpsmoe(5m)C |
| ASO-802 | 823 | moeGpsln(5m)CpsmoeApsmoeGpsmoeApsmXpsGpsTpsGpsApsApsGps(5m) |
| | | CpsGpsApsmoeApsmoeGpsmoeTpsmoeGpsln(5m)C |
| ASO-803 | 824 | moeGpsmoe(5m)CpslnApslnGpsmoeApsmXpsGpsTpsGpsApsApsGps(5m) |
| | | CpsGpsApsmoeApsmoeGpsmoeTpsmoeGpsmoe(5m)C |
| ASO-804 | 825 | moeGpsmoe(5m)CpslnApsmoeGpslnApsmXpsGpsTpsGpsApsApsGps(5m) |
| | | CpsGpsApsmoeApsmoeGpsmoeTpsmoeGpsmoe(5m)C |
| ASO-805 | 826 | moeGpsmoe(5m)CpslnApsmoeGpsmoeApsmXpsGpsTpsGpsApsApsGps(5m) |
| | | CpsGpsApslnApsmoeGpsmoeTpsmoeGpsmoe(5m)C |
| ASO-806 | 827 | moeGpsmoe(5m)CpslnApsmoeGpsmoeApsmXpsGpsTpsGpsApsApsGps(5m) |
| | | CpsGpsApsmoeApslnGpsmoeTpsmoeGpsmoe(5m)C |
| ASO-807 | 828 | moeGpsmoe(5m)CpslnApsmoeGpsmoeApsmXpsGpsTpsGpsApsApsGps(5m) |
| | | CpsGpsApsmoeApsmoeGpslnTpsmoeGpsmoe(5m)C |
| ASO-808 | 829 | moeGpsmoe(5m)CpslnApsmoeGpsmoeApsmXpsGpsTpsGpsApsApsGps(5m) |
| | | CpsGpsApsmoeApsmoeGpsmoeTpslnGpsmoe(5m)C |
| ASO-809 | 830 | moeGpsmoe(5m)CpslnApsmoeGpsmoeApsmXpsGpsTpsGpsApsApsGps(5m) |
| | | CpsGpsApsmoeApsmoeGpsmoeTpsmoeGpsln(5m)C |
| ASO-810 | 831 | moeGpsmoe(5m)CpsmoeApslnGpslnApsmXpsGpsTpsGpsApsApsGps(5m) |
| | | CpsGpsApsmoeApsmoeGpsmoeTpsmoeGpsmoe(5m)C |
| ASO-811 | 832 | moeGpsmoe(5m)CpsmoeApslnGpsmoeApsmXpsGpsTpsGpsApsApsGps(5m) |
| | | CpsGpsApslnApsmoeGpsmoeTpsmoeGpsmoe(5m)C |
| ASO-812 | 833 | moeGpsmoe(5m)CpsmoeApslnGpsmoeApsmXpsGpsTpsGpsApsApsGps(5m) |
| | | CpsGpsApsmoeApslnGpsmoeTpsmoeGpsmoe(5m)C |
| ASO-813 | 834 | moeGpsmoe(5m)CpsmoeApslnGpsmoeApsmXpsGpsTpsGpsApsApsGps(5m) |
| | | CpsGpsApsmoeApsmoeGpslnTpsmoeGpsmoe(5m)C |
| ASO-814 | 835 | moeGpsmoe(5m)CpsmoeApslnGpsmoeApsmXpsGpsTpsGpsApsApsGps(5m) |
| | | CpsGpsApsmoeApsmoeGpsmoeTpslnGpsmoe(5m)C |
| ASO-815 | 836 | moeGpsmoe(5m)CpsmoeApslnGpsmoeApsmXpsGpsTpsGpsApsApsGps(5m) |
| | | CpsGpsApsmoeApsmoeGpsmoeTpsmoeGpsln(5m)C |
| ASO-816 | 837 | moeGpsmoe(5m)CpsmoeApsmoeGpslnApsmXpsGpsTpsGpsApsApsGps(5m) |
| | | CpsGpsApslnApsmoeGpsmoeTpsmoeGpsmoe(5m)C |
| ASO-817 | 838 | moeGpsmoe(5m)CpsmoeApsmoeGpslnApsmXpsGpsTpsGpsApsApsGps(5m) |
| | | CpsGpsApsmoeApslnGpsmoeTpsmoeGpsmoe(5m)C |
| ASO-818 | 839 | moeGpsmoe(5m)CpsmoeApsmoeGpslnApsmXpsGpsTpsGpsApsApsGps(5m) |
| | | CpsGpsApsmoeApsmoeGpslnTpsmoeGpsmoe(5m)C |
| ASO-819 | 840 | moeGpsmoe(5m)CpsmoeApsmoeGpslnApsmXpsGpsTpsGpsApsApsGps(5m) |
| | | CpsGpsApsmoeApsmoeGpsmoeTpslnGpsmoe(5m)C |
| ASO-820 | 841 | moeGpsmoe(5m)CpsmoeApsmoeGpslnApsmXpsGpsTpsGpsApsApsGps(5m) |
| | | CpsGpsApsmoeApsmoeGpsmoeTpsmoeGpsln(5m)C |
| ASO-821 | 842 | moeGpsmoe(5m)CpsmoeApsmoeGpsmoeApsmXpsGpsTpsGpsApsApsGps |
| | | (5m)CpsGpsApslnApslnGpsmoeTpsmoeGpsmoe(5m)C |
| ASO-822 | 843 | moeGpsmoe(5m)CpsmoeApsmoeGpsmoeApsmXpsGpsTpsGpsApsApsGps |
| | | (5m)CpsGpsApslnApsmoeGpslnTpsmoeGpsmoe(5m)C |
| ASO-823 | 844 | moeGpsmoe(5m)CpsmoeApsmoeGpsmoeApsmXpsGpsTpsGpsApsApsGps |
| | | (5m)CpsGpsApslnApsmoeGpsmoeTpslnGpsmoe(5m)C |
| ASO-824 | 845 | moeGpsmoe(5m)CpsmoeApsmoeGpsmoeApsmXpsGpsTpsGpsApsApsGps |
| | | (5m)CpsGpsApslnApsmoeGpsmoeTpsmoeGpsln(5m)C |
| ASO-825 | 846 | moeGpsmoe(5m)CpsmoeApsmoeGpsmoeApsmXpsGpsTpsGpsApsApsGps |
| | | (5m)CpsGpsApsmoeApslnGpslnTpsmoeGpsmoe(5m)C |
| ASO-826 | 847 | moeGpsmoe(5m)CpsmoeApsmoeGpsmoeApsmXpsGpsTpsGpsApsApsGps |
| | | (5m)CpsGpsApsmoeApslnGpsmoeTpslnGpsmoe(5m)C |
| ASO-827 | 848 | moeGpsmoe(5m)CpsmoeApsmoeGpsmoeApsmXpsGpsTpsGpsApsApsGps |
| | | (5m)CpsGpsApsmoeApslnGpsmoeTpsmoeGpsln(5m)C |
| ASO-828 | 849 | moeGpsmoe(5m)CpsmoeApsmoeGpsmoeApsmXpsGpsTpsGpsApsApsGps |
| | | (5m)CpsGpsApsmoeApsmoeGpslnTpslnGpsmoe(5m)C |
| ASO-829 | 850 | moeGpsmoe(5m)CpsmoeApsmoeGpsmoeApsmXpsGpsTpsGpsApsApsGps |
| | | (5m)CpsGpsApsmoeApsmoeGpslnTpsmoeGpsln(5m)C |
| ASO-830 | 851 | moeGpsmoe(5m)CpsmoeApsmoeGpsmoeApsmXpsGpsTpsGpsApsApsGps |
| | | (5m)CpsGpsApsmoeApsmoeGpsmoeTpslnGpsln(5m)C |
| ASO-831 | 852 | moe(5m)CpsmoeApsgutbGpsmoeApsmoeGpsGpsTpsGpsApsApsGps(5m)Cps |
| | | GpsApsApsmoeGpsmoeTpsgutbGpsmoe(5m)CpsmoeA |
| ASO-832 | 853 | GpsGpsTpsGpsApsApsGps(5m)CpsGpsApsmoeApsmoeGpsmoeTpsmoeGps |
| | | moe(5m)CpsmoeApsmoe(5m)CpsmoeApsmoeApsmoeGpsmoeA |
| ASO-833 | 854 | moeApsGpsGpsTpsGpsApsApsGps(5m)CpsGpsApsmoeApsmoeGpsmoeTps |
| | | moeGpsmoe(5m)CpsmoeApsmoe(5m)CpsmoeApsmoeApsmoeG |
| ASO-834 | 855 | moeGpsmoeApsGpsGpsTpsGpsApsApsGps(5m)CpsGpsApsmoeApsmoeGps |
| | | moeTpsmoeGpsmoe(5m)CpsmoeApsmoe(5m)CpsmoeApsmoeA |
| ASO-835 | 856 | moeApsmoeGpsmoeApsGpsGpsTpsGpsApsApsGps(5m)CpsGpsApsmoeAps |
| | | moeGpsmoeTpsmoeGpsmoe(5m)CpsmoeApsmoe(5m)CpsmoeA |
| ASO-836 | 857 | moe(5m)CpsmoeApsmoeGpsmoeApsGpsGpsTpsGpsApsApsGps(5m)CpsGps |
| | | ApsmoeApsmoeGpsmoeTpsmoeGpsmoe(5m)CpsmoeApsmoe(5m)C |
| ASO-837 | 858 | moeGpsmoeTpsmoeGpsmoe(5m)CpsmoeApsmoeGpsmoeApsGpsGpsTpsGps |
| | | ApsApsGps(5m)CpsGpsApsmoeApsmoeGpsmoeTpsmoeG |
| ASO-838 | 859 | moe(5m)CpsmoeGpsmoeTpsmoeGpsmoe(5m)CpsmoeApsmoeGpsmoeAps |
| | | GpsGpsTpsGpsApsApsGps(5m)CpsGpsApsmoeApsmoeGpsmoeT |
| ASO-839 | 860 | moeApsmoe(5m)CpsmoeGpsmoeTpsmoeGpsmoe(5m)CpsmoeApsmoeGps |
| | | moeApsGpsGpsTpsGpsApsApsGps(5m)CpsGpsApsmoeApsmoeG |
| ASO-840 | 861 | GpsGpsTpsGpsmoeApsApsGps(5m)CpsGpsApsmoeApsmoeGpsmoeTpsmoe |
| | | Gpsmoe(5m)CpsmoeApsmoe(5m)CpsmoeApsmoeApsmoeGpsmoeA |
| ASO-841 | 862 | moeApsGpsGpsTpsGpsmoeApsApsGps(5m)CpsGpsApsmoeApsmoeGpsmoe |
| | | TpsmoeGpsmoe(5m)CpsmoeApsmoe(5m)CpsmoeApsmoeApsmoeG |
| ASO-842 | 863 | moeGpsmoeApsGpsGpsTpsGpsmoeApsApsGps(5m)CpsGpsApsmoeApsmoe |
| | | GpsmoeTpsmoeGpsmoe(5m)CpsmoeApsmoe(5m)CpsmoeApsmoeA |
| ASO-843 | 864 | moeApsmoeGpsmoeApsGpsGpsTpsGpsmoeApsApsGps(5m)CpsGpsApsmoe |
| | | ApsmoeGpsmoeTpsmoeGpsmoe(5m)CpsmoeApsmoe(5m)CpsmoeA |
| ASO-844 | 865 | moe(5m)CpsmoeApsmoeGpsmoeApsGpsGpsTpsGpsmoeApsApsGps(5m)Cps |
| | | GpsApsmoeApsmoeGpsmoeTpsmoeGpsmoe(5m)CpsmoeApsmoe(5m)C |
| ASO-845 | 866 | moeGpsmoe(5m)CpsmoeApsmoeGpsmoeApsGpsGpsTpsGpsmoeApsApsGps |
| | | (5m)CpsGpsApsmoeApsmoeGpsmoeTpsmoeGpsmoe(5m)CpsmoeA |
| ASO-846 | 867 | moeTpsmoeGpsmoe(5m)CpsmoeApsmoeGpsmoeApsGpsGpsTpsGpsmoeAps |
| | | ApsGps(5m)CpsGpsApsmoeApsmoeGpsmoeTpsmoeGpsmoe(5m)C |
| ASO-847 | 868 | moeGpsmoeTpsmoeGpsmoe(5m)CpsmoeApsmoeGpsmoeApsGpsGpsTpsGps |
| | | moeApsApsGps(5m)CpsGpsApsmoeApsmoeGpsmoeTpsmoeG |
| ASO-848 | 869 | moe(5m)CpsmoeGpsmoeTpsmoeGpsmoe(5m)CpsmoeApsmoeGpsmoeApsGps |
| | | GpsTpsGpsmoeApsApsGps(5m)CpsGpsApsmoeApsmoeGpsmoeT |
| ASO-849 | 870 | moeApsmoe(5m)CpsmoeGpsmoeTpsmoeGpsmoe(5m)CpsmoeApsmoeGpsmoe |
| | | ApsGpsGpsTpsGpsmoeApsApsGps(5m)CpsGpsApsmoeApsmoeG |
| ASO-850 | 871 | moe(5m)CpsmoeApsmoeGpsmoeApsmoeGpsGpsTpsGpsApsApsGps(5m)Cps |
| | | GpsApsApsmoeGpsmoeTpsmoeGpsmoe(5m)CpsmoeApsmoe(5m)C |
| ASO-851 | 872 | moe(5m)CpsmoeApsmoeGpsmoeApsmoeGpsGpsTpsGpsApsApsGps(5m)Cps |
| | | GpsApsmoeApsmoeGpsmoeTpsmoeGpsmoe(5m)CpsmoeApsmoe(5m)C |
| ASO-852 | 873 | moe(5m)CpsmoeApsmoeGpsmoeApsmoeGpsmoeGpsTpsGpsApsApsGps(5m) |
| | | CpsGpsApsApsGpsmoeTpsmoeGpsmoe(5m)CpsmoeApsmoe(5m)C |
| ASO-853 | 874 | moe(5m)CpsmoeApsmoeGpsmoeApsGpsGpsTpsGpsApsApsGps(5m)CpsGps |
| | | moeApsmoeApsmoeGpsmoeTpsmoeGpsmoe(5m)CpsmoeApsmoe(5m)C |
| ASO-854 | 875 | moe(5m)CpsmoeApsmoeGpsmoeApsmoeGpsmoeGpsmoeTpsGpsApsApsGps |
| | | (5m)CpsGpsApsApsGpsTpsmoeGpsmoe(5m)CpsmoeApsmoe(5m)C |
| ASO-855 | 876 | moe(5m)CpsmoeApsmoeGpsmoeApsmoeGpsmoeGpsTpsGpsApsApsGps(5m) |
| | | CpsGpsApsApsmoeGpsmoeTpsmoeGpsmoe(5m)CpsmoeApsmoe(5m)C |
| ASO-856 | 877 | moe(5m)CpsmoeApsmoeGpsmoeApsmoeGpsmoeGpsmoeTpsGpsApsApsGps |
| | | (5m)CpsGpsApsApsGpsmoeTpsmoeGpsmoe(5m)CpsmoeApsmoe(5m)C |
| ASO-857 | 878 | moe(5m)CpsmoeApsmoeGpsmoeApsmoeGpsmoeGpsmoeTpsmoeGpsApsAps |
| | | Gps(5m)CpsGpsApsApsGpsTpsmoeGpsmoe(5m)CpsmoeApsmoe(5m)C |
| ASO-858 | 879 | moe(5m)CpsmoeApsmoeGpsmoeApsmoeGpsmoeGpsTpsGpsApsApsGps(5m) |
| | | CpsGpsApsmoeApsmoeGpsmoeTpsmoeGpsmoe(5m)CpsmoeApsmoe(5m)C |
| ASO-859 | 880 | moe(5m)CpsmoeApsmoeGpsmoeApsmoeGpsmoeGpsmoeTpsGpsApsApsGps |
| | | (5m)CpsGpsApsApsmoeGpsmoeTpsmoeGpsmoe(5m)CpsmoeApsmoe(5m)C |
| ASO-860 | 881 | moe(5m)CpsmoeApsmoeGpsmoeApsmoeGpsGpsTpsGpsApsApsGps(5m)Cps |
| | | GpsApsApsGpsmoeTpsmoeGpsmoe(5m)CpsmoeApsmoe(5m)C |
| ASO-861 | 882 | moe(5m)CpsmoeApslnGpsmoeApsmoeGpsGpsTpsGps5ocpApsApsGps(5m) |
| | | CpsGpsApsApsmoeGpsmoeTpslnGpsmoe(5m)CpsmoeA |
| ASO-862 | 883 | moe(5m)CpsmoeApslnGpsmoeApsmoeGpsGpsTpsGps5ocpApsApsGps(5m) |
| | | CpsGpsApsApsmoeGpsmoeTpslnGpsmoe(5m)CpsmoeA |
| ASO-863 | 884 | moe(5m)CpsmoeApslnGpsmoeApsmoeGpsGpsTpsGps5omcpApsApsGps(5m) |
| | | CpsGpsApsApsmoeGpsmoeTpslnGpsmoe(5m)CpsmoeA |
| ASO-864 | 885 | moe(5m)CpsmoeApslnGpsmoeApsmoeGpsGpsTpsGps5omcpApsApsGps(5m) |
| | | CpsGpsApsApsmoeGpsmoeTpslnGpsmoe(5m)CpsmoeA |
| ASO-865 | 886 | moe(5m)CpsmoeApslnGpsmoeApsmoeGpsGpsTpsGpsAps5ocpApsGps(5m) |
| | | CpsGpsApsApsmoeGpsmoeTpslnGpsmoe(5m)CpsmoeA |
| ASO-866 | 887 | moe(5m)CpsmoeApslnGpsmoeApsmoeGpsGpsTpsGpsAps5ocpApsGps(5m) |
| | | CpsGpsApsApsmoeGpsmoeTpslnGpsmoe(5m)CpsmoeA |
| ASO-867 | 888 | moe(5m)CpsmoeApslnGpsmoeApsmoeGpsGpsTpsGpsAps5omcpApsGps(5m) |
| | | CpsGpsApsApsmoeGpsmoeTpslnGpsmoe(5m)CpsmoeA |
| ASO-868 | 889 | moe(5m)CpsmoeApslnGpsmoeApsmoeGpsGpsTpsGpsAps5omcpApsGps(5m) |
| | | CpsGpsApsApsmoeGpsmoeTpslnGpsmoe(5m)CpsmoeA |
| ASO-869 | 890 | moe(5m)CpsmoeApslnGpsmoeApsmoeGpsGpsTpsGpsApsAps5ocpGps(5m) |
| | | CpsGpsApsApsmoeGpsmoeTpslnGpsmoe(5m)CpsmoeA |
| ASO-870 | 891 | moe(5m)CpsmoeApslnGpsmoeApsmoeGpsGpsTpsGpsApsAps5omcpGps(5m) |
| | | CpsGpsApsApsmoeGpsmoeTpslnGpsmoe(5m)CpsmoeA |
| ASO-871 | 892 | moe(5m)CpsmoeApslnGpsmoeApsmoeGpsGpsTpsGpsApsApsGps(5m)Cps |
| | | 5ocpGpsApsApsmoeGpsmoeTpslnGpsmoe(5m)CpsmoeA |
| ASO-872 | 893 | moe(5m)CpsmoeApslnGpsmoeApsmoeGpsGpsTpsGpsApsApsGps(5m)Cps |
| | | 5omcpGpsApsApsmoeGpsmoeTpslnGpsmoe(5m)CpsmoeA |
| ASO-873 | 894 | moe(5m)CpsmoeApslnGpsmoeApsmoeGpsGpsTpsGpsApsApsGps(5m)Cps |
| | | Gps5ocpApsApsmoeGpsmoeTpslnGpsmoe(5m)CpsmoeA |
| ASO-874 | 895 | moe(5m)CpsmoeApslnGpsmoeApsmoeGpsGpsTpsGpsApsApsGps(5m)Cps |
| | | Gps5ocpApsApsmoeGpsmoeTpslnGpsmoe(5m)CpsmoeA |
| ASO-875 | 896 | moe(5m)CpsmoeApslnGpsmoeApsmoeGpsGpsTpsGpsApsApsGps(5m)Cps |
| | | Gps5omcpApsApsmoeGpsmoeTpslnGpsmoe(5m)CpsmoeA |
| ASO-876 | 897 | moe(5m)CpsmoeApslnGpsmoeApsmoeGpsGpsTpsGpsApsApsGps(5m)Cps |
| | | Gps5omcpApsApsmoeGpsmoeTpslnGpsmoe(5m)CpsmoeA |
| ASO-877 | 898 | moe(5m)CpsmoeApslnGpsmoeApsmoeGpsGpsTpsGpsApsApsGps(5m)Cps |
| | | GpsAps5ocpApsmoeGpsmoeTpslnGpsmoe(5m)CpsmoeA |
| ASO-878 | 899 | moe(5m)CpsmoeApslnGpsmoeApsmoeGpsGpsTpsGpsApsApsGps(5m)Cps |
| | | GpsAps5ocpApsmoeGpsmoeTpslnGpsmoe(5m)CpsmoeA |
| ASO-879 | 900 | moe(5m)CpsmoeApslnGpsmoeApsmoeGpsGpsTpsGpsApsApsGps(5m)Cps |
| | | GpsAps5omcpApsmoeGpsmoeTpslnGpsmoe(5m)CpsmoeA |
| ASO-880 | 901 | moe(5m)CpsmoeApslnGpsmoeApsmoeGpsGpsTpsGpsApsApsGps(5m)Cps |
| | | GpsAps5omcpApsmoeGpsmoeTpslnGpsmoe(5m)CpsmoeA |
| ASO-881 | 902 | moeGpsmoe(5m)CpsmoeApsmoeGpsmoeApsGpsGpsTpsGpsApsApsGps(5m) |
| | | CpsGpsGpsmoeApsmoeGpsmoeTpsmoeGpsmoe(5m)C |
| ASO-882 | 903 | moeGpsmoe(5m)CpsmoeApsmoeGpsmoeApsGpsGpsTpsGpsApsApsGps(5m) |
| | | CpsGpsTpsmoeApsmoeGpsmoeTpsmoeGpsmoe(5m)C |
| ASO-883 | 904 | moeGpsmoe(5m)CpsmoeApsmoeGpsmoeApsGpsGpsTpsGpsApsApsGps(5m) |
| | | CpsGps(5m)CpsmoeApsmoeGpsmoeTpsmoeGpsmoe(5m)C |
| ASO-884 | 905 | moeGpsmoe(5m)CpsmoeApsmoeGpsmoeApsGpsGpsTpsGpsApsApsGps(5m) |
| | | CpsApsApsmoeApsmoeGpsmoeTpsmoeGpsmoe(5m)C |
| ASO-885 | 906 | moeGpsmoe(5m)CpsmoeApsmoeGpsmoeApsGpsGpsTpsGpsApsApsGps(5m) |
| | | CpsTpsApsmoeApsmoeGpsmoeTpsmoeGpsmoe(5m)C |
| ASO-886 | 907 | moeGpsmoe(5m)CpsmoeApsmoeGpsmoeApsGpsGpsTpsGpsApsApsGps(5m) |
| | | Cps(5m)CpsApsmoeApsmoeGpsmoeTpsmoeGpsmoe(5m)C |
| ASO-887 | 908 | moeGpsmoe(5m)CpsmoeApsmoeGpsmoeApsGpsGpsTpsGpsApsApsGpsAps |
| | | GpsApsmoeApsmoeGpsmoeTpsmoeGpsmoe(5m)C |
| ASO-888 | 909 | moeGpsmoe(5m)CpsmoeApsmoeGpsmoeApsGpsGpsTpsGpsApsApsGpsTps |
| | | GpsApsmoeApsmoeGpsmoeTpsmoeGpsmoe(5m)C |
| ASO-889 | 910 | moeGpsmoe(5m)CpsmoeApsmoeGpsmoeApsGpsGpsTpsGpsApsApsGpsGps |
| | | GpsApsmoeApsmoeGpsmoeTpsmoeGpsmoe(5m)C |
| ASO-890 | 911 | moeGpsmoe(5m)CpsmoeApsmoeGpsmoeApsGpsGpsTpsGpsApsApsGps(5m) |
| | | CpsGpsApsmXpsmoeGpsmoeTpsmoeGpsmoe(5m)C |
| ASO-891 | 912 | moeGpsmoe(5m)CpsmoeApsmoeGpsmoeApsGpsGpsTpsGpsApsApsGps(5m) |
| | | CpsGpsApsmoeApsmXpsmoeTpsmoeGpsmoe(5m)C |
| ASO-892 | 913 | moeGpsmoe(5m)CpsmoeApsmoeGpsmoeApsGpsGpsTpsGpsApsApsGps(5m) |
| | | CpsGpsApsmoeApsmoeGpsmXpsmoeGpsmoe(5m)C |
| ASO-893 | 914 | moeGpsmoe(5m)CpsmoeApsmoeGpsmoeApsGpsGpsTpsGpsApsApsGps(5m) |
| | | CpsGpsApsmoeApsmoeGpsmoeTpsmoeGpsmX |
| ASO-894 | 915 | ApsGpsGpsTpsGpsmoeApsApsGps(5m)CpsGpsApsmoeApsmoeGpsmoeTps |
| | | moeGpsmoe(5m)CpsmoeApsmoe(5m)CpsmoeApsmoe(5m)CpsmoeG |
| ASO-895 | 916 | moeGpsApsGpsGpsTpsGpsmoeApsApsGps(5m)CpsGpsApsmoeApsmoeGps |
| | | moeTpsmoeGpsmoe(5m)CpsmoeApsmoe(5m)CpsmoeApsmoe(5m)C |
| ASO-896 | 917 | moeApsmoeGpsApsGpsGpsTpsGpsmoeApsApsGps(5m)CpsGpsApsmoeAps |
| | | moeGpsmoeTpsmoeGpsmoe(5m)CpsmoeApsmoe(5m)CpsmoeA |
| ASO-897 | 918 | moe(5m)CpsmoeApsmoeGpsApsGpsGpsTpsGpsmoeApsApsGps(5m)CpsGps |
| | | ApsmoeApsmoeGpsmoeTpsmoeGpsmoe(5m)CpsmoeApsmoe(5m)C |
| ASO-898 | 919 | moeGpsmoe(5m)CpsmoeApsmoeGpsApsGpsGpsTpsGpsmoeApsApsGps(5m) |
| | | CpsGpsApsmoeApsmoeGpsmoeTpsmoeGpsmoe(5m)CpsmoeA |
| ASO-899 | 920 | moeTpsmoeGpsmoe(5m)CpsmoeApsmoeGpsApsGpsGpsTpsGpsmoeApsAps |
| | | Gps(5m)CpsGpsApsmoeApsmoeGpsmoeTpsmoeGpsmoe(5m)C |
| ASO-900 | 921 | moeGpsmoeTpsmoeGpsmoe(5m)CpsmoeApsmoeGpsApsGpsGpsTpsGpsmoe |
| | | ApsApsGps(5m)CpsGpsApsmoeApsmoeGpsmoeTpsmoeG |
| ASO-901 | 922 | moe(5m)CpsmoeGpsmoeTpsmoeGpsmoe(5m)CpsmoeApsmoeGpsApsGpsGps |
| | | TpsGpsmoeApsApsGps(5m)CpsGpsApsmoeApsmoeGpsmoeT |
| ASO-902 | 923 | moeApsmoe(5m)CpsmoeGpsmoeTpsmoeGpsmoe(5m)CpsmoeApsmoeGpsAps |
| | | GpsGpsTpsGpsmoeApsApsGps(5m)CpsGpsApsmoeApsmoeG |
| ASO-903 | 924 | ApsGpsGpsTpsGpsApsApsGps(5m)CpsGpsApsmoeApsmoeGpsmoeTpsmoe |
| | | Gpsmoe(5m)CpsmoeApsmoe(5m)CpsmoeApsmoe(5m)CpsmoeG |
| ASO-904 | 925 | moeGpsApsGpsGpsTpsGpsApsApsGps(5m)CpsGpsApsmoeApsmoeGpsmoe |
| | | TpsmoeGpsmoe(5m)CpsmoeApsmoe(5m)CpsmoeApsmoe(5m)C |
| ASO-905 | 926 | moeApsmoeGpsApsGpsGpsTpsGpsApsApsGps(5m)CpsGpsApsmoeApsmoe |
| | | GpsmoeTpsmoeGpsmoe(5m)CpsmoeApsmoe(5m)CpsmoeA |
| ASO-906 | 927 | moe(5m)CpsmoeApsmoeGpsApsGpsGpsTpsGpsApsApsGps(5m)CpsGpsAps |
| | | moeApsmoeGpsmoeTpsmoeGpsmoe(5m)CpsmoeApsmoe(5m)C |
| ASO-907 | 928 | moeGpsmoe(5m)CpsmoeApsmoeGpsApsGpsGpsTpsGpsApsApsGps(5m)Cps |
| | | GpsApsmoeApsmoeGpsmoeTpsmoeGpsmoe(5m)CpsmoeA |
| ASO-908 | 929 | moeTpsmoeGpsmoe(5m)CpsmoeApsmoeGpsApsGpsGpsTpsGpsApsApsGps |
| | | (5m)CpsGpsApsmoeApsmoeGpsmoeTpsmoeGpsmoe(5m)C |
| ASO-909 | 930 | moeGpsmoeTpsmoeGpsmoe(5m)CpsmoeApsmoeGpsApsGpsGpsTpsGpsAps |
| | | ApsGps(5m)CpsGpsApsmoeApsmoeGpsmoeTpsmoeG |
| ASO-910 | 931 | moe(5m)CpsmoeGpsmoeTpsmoeGpsmoe(5m)CpsmoeApsmoeGpsApsGpsGps |
| | | TpsGpsApsApsGps(5m)CpsGpsApsmoeApsmoeGpsmoeT |
| ASO-911 | 932 | moeApsmoe(5m)CpsmoeGpsmoeTpsmoeGpsmoe(5m)CpsmoeApsmoeGpsAps |
| | | GpsGpsTpsGpsApsApsGps(5m)CpsGpsApsmoeApsmoeG |
| ASO-912 | 933 | lnXpsmoe(5m)CpsmoeApsmoeGpsmoeApsGpsGpsTpsGpsApsApsGps(5m) |
| | | CpsGpsApsmoeApsmoeGpsmoeTpsmoeGpsmoe(5m)C |
| ASO-913 | 934 | moeGpslnXpsmoeApsmoeGpsmoeApsGpsGpsTpsGpsApsApsGps(5m)Cps |
| | | GpsApsmoeApsmoeGpsmoeTpsmoeGpsmoe(5m)C |
| ASO-914 | 935 | moeGpsmoe(5m)CpslnXpsmoeGpsmoeApsGpsGpsTpsGpsApsApsGps(5m) |
| | | CpsGpsApsmoeApsmoeGpsmoeTpsmoeGpsmoe(5m)C |
| ASO-915 | 936 | moeGpsmoe(5m)CpsmoeApslnXpsmoeApsGpsGpsTpsGpsApsApsGps(5m) |
| | | CpsGpsApsmoeApsmoeGpsmoeTpsmoeGpsmoe(5m)C |
| ASO-916 | 937 | moeGpsmoe(5m)CpsmoeApsmoeGpslnXpsGpsGpsTpsGpsApsApsGps(5m) |
| | | CpsGpsApsmoeApsmoeGpsmoeTpsmoeGpsmoe(5m)C |
| ASO-917 | 938 | moeGpsmoe(5m)CpsmoeApsmoeGpsmoeApsGpsGpsTpsGpsApsApsGps(5m) |
| | | CpsGpsApslnXpsmoeGpsmoeTpsmoeGpsmoe(5m)C |
| ASO-918 | 939 | moeGpsmoe(5m)CpsmoeApsmoeGpsmoeApsGpsGpsTpsGpsApsApsGps(5m) |
| | | CpsGpsApsmoeApslnXpsmoeTpsmoeGpsmoe(5m)C |
| ASO-919 | 940 | moeGpsmoe(5m)CpsmoeApsmoeGpsmoeApsGpsGpsTpsGpsApsApsGps(5m) |
| | | CpsGpsApsmoeApsmoeGpslnXpsmoeGpsmoe(5m)C |
| ASO-920 | 941 | moeGpsmoe(5m)CpsmoeApsmoeGpsmoeApsGpsGpsTpsGpsApsApsGps(5m) |
| | | CpsGpsApsmoeApsmoeGpsmoeTpslnXpsmoe(5m)C |
| ASO-921 | 942 | moeGpsmoe(5m)CpsmoeApsmoeGpsmoeApsGpsGpsTpsGpsApsApsGps(5m) |
| | | CpsGpsApsmoeApsmoeGpsmoeTpsmoeGpslnX |
| ASO-922 | 943 | moeXpsmoe(5m)CpsmoeApsmoeGpsmoeApsGpsGpsTpsGpsApsApsGps(5m) |
| | | CpsGpsApsmoeApsmoeGpsmoeTpsmoeGpsmoe(5m)C |
| ASO-923 | 944 | moeGpsmoeXpsmoeApsmoeGpsmoeApsGpsGpsTpsGpsApsApsGps(5m)Cps |
| | | GpsApsmoeApsmoeGpsmoeTpsmoeGpsmoe(5m)C |
| ASO-924 | 945 | moeGpsmoe(5m)CpsmoeXpsmoeGpsmoeApsGpsGpsTpsGpsApsApsGps(5m) |
| | | CpsGpsApsmoeApsmoeGpsmoeTpsmoeGpsmoe(5m)C |
| ASO-925 | 946 | moeGpsmoe(5m)CpsmoeApsmoeXpsmoeApsGpsGpsTpsGpsApsApsGps(5m) |
| | | CpsGpsApsmoeApsmoeGpsmoeTpsmoeGpsmoe(5m)C |
| ASO-926 | 947 | moeGpsmoe(5m)CpsmoeApsmoeGpsmoeXpsGpsGpsTpsGpsApsApsGps(5m) |
| | | CpsGpsApsmoeApsmoeGpsmoeTpsmoeGpsmoe(5m)C |
| ASO-927 | 948 | moeGpsmoe(5m)CpsmoeApsmoeGpsmoeApsGpsGpsTpsGpsApsApsGps(5m) |
| | | CpsGpsApsmoeXpsmoeGpsmoeTpsmoeGpsmoe(5m)C |
| ASO-928 | 949 | moeGpsmoe(5m)CpsmoeApsmoeGpsmoeApsGpsGpsTpsGpsApsApsGps(5m) |
| | | CpsGpsApsmoeApsmoeXpsmoeTpsmoeGpsmoe(5m)C |
| ASO-929 | 950 | moeGpsmoe(5m)CpsmoeApsmoeGpsmoeApsGpsGpsTpsGpsApsApsGps(5m) |
| | | CpsGpsApsmoeApsmoeGpsmoeXpsmoeGpsmoe(5m)C |
| ASO-930 | 951 | moeGpsmoe(5m)CpsmoeApsmoeGpsmoeApsGpsGpsTpsGpsApsApsGps(5m) |
| | | CpsGpsApsmoeApsmoeGpsmoeTpsmoeXpsmoe(5m)C |
| ASO-931 | 952 | moeGpsmoe(5m)CpsmoeApsmoeGpsmoeApsGpsGpsTpsGpsApsApsGps(5m) |
| | | CpsGpsApsmoeApsmoeGpsmoeTpsmoeGpsmoeX |
| ASO-932 | 953 | moeGpsmoe(5m)CpsmoeApsmoeGpsApsGpsGpsTpsGpsmoeXpsApsGps(5m) |
| | | CpsGpsApsmoeApslnGpsmoeTpslnGpsln(5m)C |
| ASO-933 | 954 | moeGpsmoe(5m)CpsmoeApsmoeGpsApsGpsGpsTpsGpslnXpsApsGps(5m) |
| | | CpsGpsApsmoeApslnGpsmoeTpslnGpsln(5m)C |
| ASO-934 | 955 | moeGpsmoe(5m)CpslnApsmoeGpslnApsGpsGpsTpsGpsApsAps(5m)Cps(5m) |
| | | CpsGpsApsmoeApsmoeGpsmoeTpsmoeGpsmoe(5m)C |
| ASO-935 | 956 | moeGpsmoe(5m)CpsmoeApslnGpslnApsGpsGpsTpsGpsApsAps(5m)Cps(5m) |
| | | CpsGpsApsmoeApsmoeGpsmoeTpsmoeGpsmoe(5m)C |
| ASO-936 | 957 | moeGpsmoe(5m)CpsmoeApslnGpsmoeApsGpsGpsTpsGpsApsAps(5m)Cps |
| | | (5m)CpsGpsApslnApsmoeGpsmoeTpsmoeGpsmoe(5m)C |
| ASO-937 | 958 | moeGpsmoe(5m)CpsmoeApsmoeGpslnApsGpsGpsTpsGpsApsAps(5m)Cps |
| | | (5m)CpsGpsApslnApsmoeGpsmoeTpsmoeGpsmoe(5m)C |
| ASO-938 | 959 | moeGpsmoe(5m)CpsmoeApsmoeGpslnApsGpsGpsTpsGpsApsAps(5m)Cps |
| | | (5m)CpsGpsApsmoeApsmoeGpsmoeTpslnGpsmoe(5m)C |
| ASO-939 | 960 | moeGpsmoe(5m)CpsmoeApsmoeGpsmoeApsGpsGpsTpsGpsApsAps(5m)Cps |
| | | (5m)CpsGpsApslnApsmoeGpslnTpsmoeGpsmoe(5m)C |
| ASO-940 | 961 | moeGpsmoe(5m)CpslnApsmoeGpslnApsGpsGpsTpsGpsApsApsTps(5m)Cps |
| | | GpsApsmoeApsmoeGpsmoeTpsmoeGpsmoe(5m)C |
| ASO-941 | 962 | moeGpsmoe(5m)CpsmoeApslnGpslnApsGpsGpsTpsGpsApsApsTps(5m)Cps |
| | | GpsApsmoeApsmoeGpsmoeTpsmoeGpsmoe(5m)C |
| ASO-942 | 963 | moeGpsmoe(5m)CpsmoeApslnGpsmoeApsGpsGpsTpsGpsApsApsTps(5m) |
| | | CpsGpsApslnApsmoeGpsmoeTpsmoeGpsmoe(5m)C |
| ASO-943 | 964 | moeGpsmoe(5m)CpsmoeApsmoeGpslnApsGpsGpsTpsGpsApsApsTps(5m) |
| | | CpsGpsApslnApsmoeGpsmoeTpsmoeGpsmoe(5m)C |
| ASO-944 | 965 | moeGpsmoe(5m)CpsmoeApsmoeGpslnApsGpsGpsTpsGpsApsApsTps(5m) |
| | | CpsGpsApsmoeApsmoeGpsmoeTpslnGpsmoe(5m)C |
| ASO-945 | 966 | moeGpsmoe(5m)CpsmoeApsmoeGpsmoeApsGpsGpsTpsGpsApsApsTps(5m) |
| | | CpsGpsApslnApsmoeGpslnTpsmoeGpsmoe(5m)C |
| ASO-946 | 967 | moeGpsmoe(5m)CpsmoeApsmoeGpslnApsGpsGpsTps(5m)CpsApsApsGps |
| | | (5m)CpsGpsApsmoeApsmoeGpsmoeTpsmoeGpsmoe(5m)C |
| ASO-947 | 968 | moeGpsmoe(5m)CpsmoeApsmoeGpsmoeApsGpsGpsTps(5m)CpsApsApsGps |
| | | (5m)CpsGpsApsmoeApslnGpsmoeTpsmoeGpsmoe(5m)C |
| ASO-948 | 969 | moeGpsmoe(5m)CpsmoeApsmoeGpslnApsGpsGpsTpsGpsGpsApsGps(5m) |
| | | CpsGpsApsmoeApsmoeGpsmoeTpsmoeGpsmoe(5m)C |
| ASO-949 | 970 | moeGpsmoe(5m)CpsmoeApsmoeGpsmoeApsGpsGpsTpsGpsGpsApsGps(5m) |
| | | CpsGpsApsmoeApslnGpsmoeTpsmoeGpsmoe(5m)C |
| ASO-950 | 971 | moeGpsmoe(5m)CpsmoeApsmoeGpslnApsGpsGpsTpsGpsApsTpsGps(5m) |
| | | CpsGpsApsmoeApsmoeGpsmoeTpsmoeGpsmoe(5m)C |
| ASO-951 | 972 | moeGpsmoe(5m)CpsmoeApsmoeGpsmoeApsGpsGpsTpsGpsApsTpsGps(5m) |
| | | CpsGpsApsmoeApslnGpsmoeTpsmoeGpsmoe(5m)C |
| ASO-952 | 973 | moeGpsmoe(5m)CpsmoeApsmoeGpslnApsGpsGpsTpsGpsApsAps(5m)Cps |
| | | (5m)CpsGpsApsmoeApsmoeGpsmoeTpsmoeGpsmoe(5m)C |
| ASO-953 | 974 | moeGpsmoe(5m)CpsmoeApsmoeGpsmoeApsGpsGpsTpsGpsApsAps(5m)Cps |
| | | (5m)CpsGpsApsmoeApslnGpsmoeTpsmoeGpsmoe(5m)C |
| ASO-954 | 975 | moeGpsmoe(5m)CpsmoeApsmoeGpslnApsGpsGpsTpsGpsApsApsTps(5m) |
| | | CpsGpsApsmoeApsmoeGpsmoeTpsmoeGpsmoe(5m)C |
| ASO-955 | 976 | moeGpsmoe(5m)CpsmoeApsmoeGpsmoeApsGpsGpsTpsGpsApsApsTps(5m) |
| | | CpsGpsApsmoeApslnGpsmoeTpsmoeGpsmoe(5m)C |
| ASO-956 | 977 | moeGpsmoe(5m)CpsmoeApsmoeGpslnApsGpsGpsTpsGpsApsmXpsGps(5m) |
| | | CpsGpsApsmoeApsmoeGpsmoeTpsmoeGpsmoe(5m)C |
| ASO-957 | 978 | moeGpsmoe(5m)CpsmoeApsmoeGpsmoeApsGpsGpsTpsGpsApsmXpsGps |
| | | (5m)CpsGpsApsmoeApslnGpsmoeTpsmoeGpsmoe(5m)C |
| ASO-958 | 979 | moeGpsmoe(5m)CpsmoeApsmoeGpslnApsGpsGpsTpsGpsdXpsApsGps(5m) |
| | | CpsGpsApsmoeApsmoeGpsmoeTpsmoeGpsmoe(5m)C |
| ASO-959 | 980 | moeGpsmoe(5m)CpsmoeApsmoeGpsmoeApsGpsGpsTpsGpsdXpsApsGps |
| | | (5m)CpsGpsApsmoeApslnGpsmoeTpsmoeGpsmoe(5m)C |
| ASO-960 | 981 | moeGpsmoe(5m)CpslnApsmoeGpslnApsGpsGpsTpsGpsApsmXpsGps(5m) |
| | | CpsGpsApsmoeApsmoeGpsmoeTpsmoeGpsmoe(5m)C |
| ASO-961 | 982 | moeGpsmoe(5m)CpsmoeApslnGpslnApsGpsGpsTpsGpsApsmXpsGps(5m) |
| | | CpsGpsApsmoeApsmoeGpsmoeTpsmoeGpsmoe(5m)C |
| ASO-962 | 983 | moeGpsmoe(5m)CpsmoeApslnGpsmoeApsGpsGpsTpsGpsApsmXpsGps(5m) |
| | | CpsGpsApslnApsmoeGpsmoeTpsmoeGpsmoe(5m)C |
| ASO-963 | 984 | moeGpsmoe(5m)CpsmoeApsmoeGpslnApsGpsGpsTpsGpsApsmXpsGps(5m) |
| | | CpsGpsApslnApsmoeGpsmoeTpsmoeGpsmoe(5m)C |
| ASO-964 | 985 | moeGpsmoe(5m)CpsmoeApsmoeGpslnApsGpsGpsTpsGpsApsmXpsGps(5m) |
| | | CpsGpsApsmoeApsmoeGpsmoeTpslnGpsmoe(5m)C |
| ASO-965 | 986 | moeGpsmoe(5m)CpsmoeApsmoeGpsmoeApsGpsGpsTpsGpsApsmXpsGps |
| | | (5m)CpsGpsApslnApsmoeGpslnTpsmoeGpsmoe(5m)C |
| ASO-966 | 987 | moeGpsmoe(5m)CpslnApsmoeGpslnApsGpsGpsTpsGpsGpsApsGps(5m)Cps |
| | | GpsApsmoeApsmoeGpsmoeTpsmoeGpsmoe(5m)C |
| ASO-967 | 988 | moeGpsmoe(5m)CpsmoeApslnGpslnApsGpsGpsTpsGpsGpsApsGps(5m)Cps |
| | | GpsApsmoeApsmoeGpsmoeTpsmoeGpsmoe(5m)C |
| ASO-968 | 989 | moeGpsmoe(5m)CpsmoeApslnGpsmoeApsGpsGpsTpsGpsGpsApsGps(5m) |
| | | CpsGpsApslnApsmoeGpsmoeTpsmoeGpsmoe(5m)C |
| ASO-969 | 990 | moeGpsmoe(5m)CpsmoeApsmoeGpslnApsGpsGpsTpsGpsGpsApsGps(5m) |
| | | CpsGpsApslnApsmoeGpsmoeTpsmoeGpsmoe(5m)C |
| ASO-970 | 991 | moeGpsmoe(5m)CpsmoeApsmoeGpslnApsGpsGpsTpsGpsGpsApsGps(5m) |
| | | CpsGpsApsmoeApsmoeGpsmoeTpslnGpsmoe(5m)C |
| ASO-971 | 992 | moeGpsmoe(5m)CpsmoeApsmoeGpsmoeApsGpsGpsTpsGpsGpsApsGps(5m) |
| | | CpsGpsApslnApsmoeGpslnTpsmoeGpsmoe(5m)C |
| ASO-972 | 993 | moeGpsmoe(5m)CpslnApsmoeGpslnApsGpsGpsTps(5m)CpsApsApsGps(5m) |
| | | CpsGpsApsmoeApsmoeGpsmoeTpsmoeGpsmoe(5m)C |
| ASO-973 | 994 | moeGpsmoe(5m)CpsmoeApslnGpslnApsGpsGpsTps(5m)CpsApsApsGps(5m) |
| | | CpsGpsApsmoeApsmoeGpsmoeTpsmoeGpsmoe(5m)C |
| ASO-974 | 995 | moeGpsmoe(5m)CpsmoeApslnGpsmoeApsGpsGpsTps(5m)CpsApsApsGps |
| | | (5m)CpsGpsApslnApsmoeGpsmoeTpsmoeGpsmoe(5m)C |
| ASO-975 | 996 | moeGpsmoe(5m)CpsmoeApsmoeGpslnApsGpsGpsTps(5m)CpsApsApsGps |
| | | (5m)CpsGpsApslnApsmoeGpsmoeTpsmoeGpsmoe(5m)C |
| ASO-976 | 997 | moeGpsmoe(5m)CpsmoeApsmoeGpslnApsGpsGpsTps(5m)CpsApsApsGps |
| | | (5m)CpsGpsApsmoeApsmoeGpsmoeTpslnGpsmoe(5m)C |
| ASO-977 | 998 | moeGpsmoe(5m)CpsmoeApsmoeGpsmoeApsGpsGpsTps(5m)CpsApsApsGps |
| | | (5m)CpsGpsApslnApsmoeGpslnTpsmoeGpsmoe(5m)C |
| ASO-978 | 999 | moeGpsmoe(5m)CpslnApsmoeGpslnApsGpsGpsTpsGpsApsTpsGps(5m)Cps |
| | | GpsApsmoeApsmoeGpsmoeTpsmoeGpsmoe(5m)C |
| ASO-979 | 1000 | moeGpsmoe(5m)CpsmoeApslnGpslnApsGpsGpsTpsGpsApsTpsGps(5m)Cps |
| | | GpsApsmoeApsmoeGpsmoeTpsmoeGpsmoe(5m)C |
| ASO-980 | 1001 | moeGpsmoe(5m)CpsmoeApslnGpsmoeApsGpsGpsTpsGpsApsTpsGps(5m) |
| | | CpsGpsApslnApsmoeGpsmoeTpsmoeGpsmoe(5m)C |
| ASO-981 | 1002 | moeGpsmoe(5m)CpsmoeApsmoeGpslnApsGpsGpsTpsGpsApsTpsGps(5m) |
| | | CpsGpsApslnApsmoeGpsmoeTpsmoeGpsmoe(5m)C |
| ASO-982 | 1003 | moeGpsmoe(5m)CpsmoeApsmoeGpslnApsGpsGpsTpsGpsApsTpsGps(5m) |
| | | CpsGpsApsmoeApsmoeGpsmoeTpslnGpsmoe(5m)C |
| ASO-983 | 1004 | moeGpsmoe(5m)CpsmoeApsmoeGpsmoeApsGpsGpsTpsGpsApsTpsGps(5m) |
| | | CpsGpsApslnApsmoeGpslnTpsmoeGpsmoe(5m)C |
| ASO-984 | 1005 | moeGpsmoe(5m)CpslnApsmoeGpslnApsGpsGpsTpsGpsdXpsApsGps(5m)Cps |
| | | GpsApsmoeApsmoeGpsmoeTpsmoeGpsmoe(5m)C |
| ASO-985 | 1006 | moeGpsmoe(5m)CpsmoeApslnGpslnApsGpsGpsTpsGpsdXpsApsGps(5m)Cps |
| | | GpsApsmoeApsmoeGpsmoeTpsmoeGpsmoe(5m)C |
| ASO-986 | 1007 | moeGpsmoe(5m)CpsmoeApslnGpsmoeApsGpsGpsTpsGpsdXpsApsGps(5m) |
| | | CpsGpsApslnApsmoeGpsmoeTpsmoeGpsmoe(5m)C |
| ASO-987 | 1008 | moeGpsmoe(5m)CpsmoeApsmoeGpslnApsGpsGpsTpsGpsdXpsApsGps(5m) |
| | | CpsGpsApslnApsmoeGpsmoeTpsmoeGpsmoe(5m)C |
| ASO-988 | 1009 | moeGpsmoe(5m)CpsmoeApsmoeGpslnApsGpsGpsTpsGpsdXpsApsGps(5m) |
| | | CpsGpsApsmoeApsmoeGpsmoeTpslnGpsmoe(5m)C |
| ASO-989 | 1010 | moeGpsmoe(5m)CpsmoeApsmoeGpsmoeApsGpsGpsTpsGpsdXpsApsGps |
| | | (5m)CpsGpsApslnApsmoeGpslnTpsmoeGpsmoe(5m)C |
| ASO-990 | 1011 | moe(5m)CpsmoeApsmoeGpsmoeApsmoeGpsmXpsTpsGpsApsApsGps(5m) |
| | | CpsGpsApsApslnGpsmoeTpslnGpsmoe(5m)CpslnA |
| ASO-991 | 1012 | moe(5m)CpsmoeApsmoeGpsmoeApsmoeGpsGpsmXpsGpsApsApsGps(5m) |
| | | CpsGpsApsApslnGpsmoeTpslnGpsmoe(5m)CpslnA |
| ASO-992 | 1013 | moe(5m)CpsmoeApsmoeGpsmoeApsmoeGpsGpsTpsmXpsApsApsGps(5m) |
| | | CpsGpsApsApslnGpsmoeTpslnGpsmoe(5m)CpslnA |
| ASO-993 | 1014 | moe(5m)CpsmoeApsmoeGpsmoeApsmoeGpsGpsTpsGpsmXpsApsGps(5m) |
| | | CpsGpsApsApslnGpsmoeTpslnGpsmoe(5m)CpslnA |
| ASO-994 | 1015 | moe(5m)CpsmoeApsmoeGpsmoeApsmoeGpsGpsTpsGpsApsmXpsGps(5m) |
| | | CpsGpsApsApslnGpsmoeTpslnGpsmoe(5m)CpslnA |
| ASO-995 | 1016 | moe(5m)CpsmoeApsmoeGpsmoeApsmoeGpsGpsTpsGpsApsApsmXps(5m) |
| | | CpsGpsApsApslnGpsmoeTpslnGpsmoe(5m)CpslnA |
| ASO-996 | 1017 | moe(5m)CpsmoeApsmoeGpsmoeApsmoeGpsGpsTpsGpsApsApsGpsmXps |
| | | GpsApsApslnGpsmoeTpslnGpsmoe(5m)CpslnA |
| ASO-997 | 1018 | moe(5m)CpsmoeApsmoeGpsmoeApsmoeGpsGpsTpsGpsApsApsGps(5m) |
| | | CpsmXpsApsApslnGpsmoeTpslnGpsmoe(5m)CpslnA |
| ASO-998 | 1019 | moe(5m)CpsmoeApsmoeGpsmoeApsmoeGpsGpsTpsGpsApsApsGps(5m)Cps |
| | | GpsmXpsApslnGpsmoeTpslnGpsmoe(5m)CpslnA |
| ASO-999 | 1020 | moe(5m)CpsmoeApsmoeGpsmoeApsmoeGpsGpsTpsGpsApsApsGps(5m)Cps |
| | | GpsApsmXpslnGpsmoeTpslnGpsmoe(5m)CpslnA |
| ASO-1000 | 1021 | lnGpsln(5m)CpsmoeApsmoeGpsmoeApsGpsGpsTpsGpsApsmXpsGps(5m) |
| | | CpsGpsApsmoeApsmoeGpsmoeTpsmoeGpsmoe(5m)C |
| ASO-1001 | 1022 | lnGpsmoe(5m)CpslnApsmoeGpsmoeApsGpsGpsTpsGpsApsmXpsGps(5m) |
| | | CpsGpsApsmoeApsmoeGpsmoeTpsmoeGpsmoe(5m)C |
| ASO-1002 | 1023 | lnGpsmoe(5m)CpsmoeApslnGpsmoeApsGpsGpsTpsGpsApsmXpsGps(5m) |
| | | CpsGpsApsmoeApsmoeGpsmoeTpsmoeGpsmoe(5m)C |
| ASO-1003 | 1024 | lnGpsmoe(5m)CpsmoeApsmoeGpslnApsGpsGpsTpsGpsApsmXpsGps(5m) |
| | | CpsGpsApsmoeApsmoeGpsmoeTpsmoeGpsmoe(5m)C |
| ASO-1004 | 1025 | lnGpsmoe(5m)CpsmoeApsmoeGpsmoeApsGpsGpsTpsGpsApsmXpsGps(5m) |
| | | CpsGpsApslnApsmoeGpsmoeTpsmoeGpsmoe(5m)C |
| ASO-1005 | 1026 | lnGpsmoe(5m)CpsmoeApsmoeGpsmoeApsGpsGpsTpsGpsApsmXpsGps(5m) |
| | | CpsGpsApsmoeApslnGpsmoeTpsmoeGpsmoe(5m)C |
| ASO-1006 | 1027 | lnGpsmoe(5m)CpsmoeApsmoeGpsmoeApsGpsGpsTpsGpsApsmXpsGps(5m) |
| | | CpsGpsApsmoeApsmoeGpslnTpsmoeGpsmoe(5m)C |
| ASO-1007 | 1028 | lnGpsmoe(5m)CpsmoeApsmoeGpsmoeApsGpsGpsTpsGpsApsmXpsGps(5m) |
| | | CpsGpsApsmoeApsmoeGpsmoeTpslnGpsmoe(5m)C |
| ASO-1008 | 1029 | lnGpsmoe(5m)CpsmoeApsmoeGpsmoeApsGpsGpsTpsGpsApsmXpsGps(5m) |
| | | CpsGpsApsmoeApsmoeGpsmoeTpsmoeGpsln(5m)C |
| ASO-1009 | 1030 | moeGpsln(5m)CpslnApsmoeGpsmoeApsGpsGpsTpsGpsApsmXpsGps(5m)Cps |
| | | GpsApsmoeApsmoeGpsmoeTpsmoeGpsmoe(5m)C |
| ASO-1010 | 1031 | moeGpsln(5m)CpsmoeApslnGpsmoeApsGpsGpsTpsGpsApsmXpsGps(5m)Cps |
| | | GpsApsmoeApsmoeGpsmoeTpsmoeGpsmoe(5m)C |
| ASO-1011 | 1032 | moeGpsln(5m)CpsmoeApsmoeGpslnApsGpsGpsTpsGpsApsmXpsGps(5m)Cps |
| | | GpsApsmoeApsmoeGpsmoeTpsmoeGpsmoe(5m)C |
| ASO-1012 | 1033 | moeGpsln(5m)CpsmoeApsmoeGpsmoeApsGpsGpsTpsGpsApsmXpsGps(5m) |
| | | CpsGpsApslnApsmoeGpsmoeTpsmoeGpsmoe(5m)C |
| ASO-1013 | 1034 | moeGpsln(5m)CpsmoeApsmoeGpsmoeApsGpsGpsTpsGpsApsmXpsGps(5m) |
| | | CpsGpsApsmoeApslnGpsmoeTpsmoeGpsmoe(5m)C |
| ASO-1014 | 1035 | moeGpsln(5m)CpsmoeApsmoeGpsmoeApsGpsGpsTpsGpsApsmXpsGps(5m) |
| | | CpsGpsApsmoeApsmoeGpslnTpsmoeGpsmoe(5m)C |
| ASO-1015 | 1036 | moeGpsln(5m)CpsmoeApsmoeGpsmoeApsGpsGpsTpsGpsApsmXpsGps(5m) |
| | | CpsGpsApsmoeApsmoeGpsmoeTpslnGpsmoe(5m)C |
| ASO-1016 | 1037 | moeGpsln(5m)CpsmoeApsmoeGpsmoeApsGpsGpsTpsGpsApsmXpsGps(5m) |
| | | CpsGpsApsmoeApsmoeGpsmoeTpsmoeGpsln(5m)C |
| ASO-1017 | 1038 | moeGpsmoe(5m)CpslnApslnGpsmoeApsGpsGpsTpsGpsApsmXpsGps(5m)Cps |
| | | GpsApsmoeApsmoeGpsmoeTpsmoeGpsmoe(5m)C |
| ASO-1018 | 1039 | moeGpsmoe(5m)CpslnApsmoeGpsmoeApsGpsGpsTpsGpsApsmXpsGps(5m) |
| | | CpsGpsApslnApsmoeGpsmoeTpsmoeGpsmoe(5m)C |
| ASO-1019 | 1040 | moeGpsmoe(5m)CpslnApsmoeGpsmoeApsGpsGpsTpsGpsApsmXpsGps(5m) |
| | | CpsGpsApsmoeApslnGpsmoeTpsmoeGpsmoe(5m)C |
| ASO-1020 | 1041 | moeGpsmoe(5m)CpslnApsmoeGpsmoeApsGpsGpsTpsGpsApsmXpsGps(5m) |
| | | CpsGpsApsmoeApsmoeGpslnTpsmoeGpsmoe(5m)C |
| ASO-1021 | 1042 | moeGpsmoe(5m)CpslnApsmoeGpsmoeApsGpsGpsTpsGpsApsmXpsGps(5m) |
| | | CpsGpsApsmoeApsmoeGpsmoeTpslnGpsmoe(5m)C |
| ASO-1022 | 1043 | moeGpsmoe(5m)CpslnApsmoeGpsmoeApsGpsGpsTpsGpsApsmXpsGps(5m) |
| | | CpsGpsApsmoeApsmoeGpsmoeTpsmoeGpsln(5m)C |
| ASO-1023 | 1044 | moeGpsmoe(5m)CpsmoeApslnGpsmoeApsGpsGpsTpsGpsApsmXpsGps(5m) |
| | | CpsGpsApsmoeApslnGpsmoeTpsmoeGpsmoe(5m)C |
| ASO-1024 | 1045 | moeGpsmoe(5m)CpsmoeApslnGpsmoeApsGpsGpsTpsGpsApsmXpsGps(5m) |
| | | CpsGpsApsmoeApsmoeGpslnTpsmoeGpsmoe(5m)C |
| ASO-1025 | 1046 | moeGpsmoe(5m)CpsmoeApslnGpsmoeApsGpsGpsTpsGpsApsmXpsGps(5m) |
| | | CpsGpsApsmoeApsmoeGpsmoeTpslnGpsmoe(5m)C |
| ASO-1026 | 1047 | moeGpsmoe(5m)CpsmoeApslnGpsmoeApsGpsGpsTpsGpsApsmXpsGps(5m) |
| | | CpsGpsApsmoeApsmoeGpsmoeTpsmoeGpsln(5m)C |
| ASO-1027 | 1048 | moeGpsmoe(5m)CpsmoeApsmoeGpslnApsGpsGpsTpsGpsApsmXpsGps(5m) |
| | | CpsGpsApsmoeApslnGpsmoeTpsmoeGpsmoe(5m)C |
| ASO-1028 | 1049 | moeGpsmoe(5m)CpsmoeApsmoeGpslnApsGpsGpsTpsGpsApsmXpsGps(5m) |
| | | CpsGpsApsmoeApsmoeGpslnTpsmoeGpsmoe(5m)C |
| ASO-1029 | 1050 | moeGpsmoe(5m)CpsmoeApsmoeGpslnApsGpsGpsTpsGpsApsmXpsGps(5m) |
| | | CpsGpsApsmoeApsmoeGpsmoeTpsmoeGpsln(5m)C |
| ASO-1030 | 1051 | moeGpsmoe(5m)CpsmoeApsmoeGpsmoeApsGpsGpsTpsGpsApsmXpsGps |
| | | (5m)CpsGpsApslnApslnGpsmoeTpsmoeGpsmoe(5m)C |
| ASO-1031 | 1052 | moeGpsmoe(5m)CpsmoeApsmoeGpsmoeApsGpsGpsTpsGpsApsmXpsGps |
| | | (5m)CpsGpsApslnApsmoeGpsmoeTpslnGpsmoe(5m)C |
| ASO-1032 | 1053 | moeGpsmoe(5m)CpsmoeApsmoeGpsmoeApsGpsGpsTpsGpsApsmXpsGps |
| | | (5m)CpsGpsApslnApsmoeGpsmoeTpsmoeGpsln(5m)C |
| ASO-1033 | 1054 | moeGpsmoe(5m)CpsmoeApsmoeGpsmoeApsGpsGpsTpsGpsApsmXpsGps |
| | | (5m)CpsGpsApsmoeApslnGpslnTpsmoeGpsmoe(5m)C |
| ASO-1034 | 1055 | moeGpsmoe(5m)CpsmoeApsmoeGpsmoeApsGpsGpsTpsGpsApsmXpsGps |
| | | (5m)CpsGpsApsmoeApslnGpsmoeTpslnGpsmoe(5m)C |
| ASO-1035 | 1056 | moeGpsmoe(5m)CpsmoeApsmoeGpsmoeApsGpsGpsTpsGpsApsmXpsGps |
| | | (5m)CpsGpsApsmoeApslnGpsmoeTpsmoeGpsln(5m)C |
| ASO-1036 | 1057 | moeGpsmoe(5m)CpsmoeApsmoeGpsmoeApsGpsGpsTpsGpsApsmXpsGps |
| | | (5m)CpsGpsApsmoeApsmoeGpslnTpslnGpsmoe(5m)C |
| ASO-1037 | 1058 | moeGpsmoe(5m)CpsmoeApsmoeGpsmoeApsGpsGpsTpsGpsApsmXpsGps |
| | | (5m)CpsGpsApsmoeApsmoeGpslnTpsmoeGpsln(5m)C |
| ASO-1038 | 1059 | moeGpsmoe(5m)CpsmoeApsmoeGpsmoeApsGpsGpsTpsGpsApsmXpsGps |
| | | (5m)CpsGpsApsmoeApsmoeGpsmoeTpslnGpsln(5m)C |
| ASO-1039 | 1060 | lnGpsmoe(5m)CpsmoeApsmoeGpsmoeApsGpsGpsTpsGpsApsmXpsGps(5m) |
| | | CpsGpsApsmoeApsmoeGpsmoeTpsmoeGpsmoe(5m)C |
| ASO-1040 | 1061 | moeGpsln(5m)CpsmoeApsmoeGpsmoeApsGpsGpsTpsGpsApsmXpsGps(5m) |
| | | CpsGpsApsmoeApsmoeGpsmoeTpsmoeGpsmoe(5m)C |
| ASO-1041 | 1062 | moeGpsmoe(5m)CpslnApsmoeGpsmoeApsGpsGpsTpsGpsApsmXpsGps(5m) |
| | | CpsGpsApsmoeApsmoeGpsmoeTpsmoeGpsmoe(5m)C |
| ASO-1042 | 1063 | moeGpsmoe(5m)CpsmoeApslnGpsmoeApsGpsGpsTpsGpsApsmXpsGps(5m) |
| | | CpsGpsApsmoeApsmoeGpsmoeTpsmoeGpsmoe(5m)C |
| ASO-1043 | 1064 | moeGpsmoe(5m)CpsmoeApsmoeGpsmoeApsGpsGpsTpsGpsApsmXpsGps |
| | | (5m)CpsGpsApslnApsmoeGpsmoeTpsmoeGpsmoe(5m)C |
| ASO-1044 | 1065 | moeGpsmoe(5m)CpsmoeApsmoeGpsmoeApsGpsGpsTpsGpsApsmXpsGps |
| | | (5m)CpsGpsApsmoeApsmoeGpslnTpsmoeGpsmoe(5m)C |
| ASO-1045 | 1066 | moeGpsmoe(5m)CpsmoeApsmoeGpsmoeApsGpsGpsTpsGpsApsmXpsGps |
| | | (5m)CpsGpsApsmoeApsmoeGpsmoeTpslnGpsmoe(5m)C |
| ASO-1046 | 1067 | moeGpsmoe(5m)CpsmoeApsmoeGpsmoeApsGpsGpsTpsGpsApsmXpsGps |
| | | (5m)CpsGpsApsmoeApsmoeGpsmoeTpsmoeGpsln(5m)C |
| ASO-1047 | 1068 | lnGpsmoe(5m)CpsmoeApsmoeGpsmoeApsGpsGpsTpsGpsTpsApsGps(5m) |
| | | CpsGpsApsmoeApsmoeGpsmoeTpsmoeGpsmoe(5m)C |
| ASO-1048 | 1069 | moeGpsln(5m)CpsmoeApsmoeGpsmoeApsGpsGpsTpsGpsTpsApsGps(5m) |
| | | CpsGpsApsmoeApsmoeGpsmoeTpsmoeGpsmoe(5m)C |
| ASO-1049 | 1070 | moeGpsmoe(5m)CpslnApsmoeGpsmoeApsGpsGpsTpsGpsTpsApsGps(5m) |
| | | CpsGpsApsmoeApsmoeGpsmoeTpsmoeGpsmoe(5m)C |
| ASO-1050 | 1071 | moeGpsmoe(5m)CpsmoeApslnGpsmoeApsGpsGpsTpsGpsTpsApsGps(5m) |
| | | CpsGpsApsmoeApsmoeGpsmoeTpsmoeGpsmoe(5m)C |
| ASO-1051 | 1072 | moeGpsmoe(5m)CpsmoeApsmoeGpslnApsGpsGpsTpsGpsTpsApsGps(5m) |
| | | CpsGpsApsmoeApsmoeGpsmoeTpsmoeGpsmoe(5m)C |
| ASO-1052 | 1073 | moeGpsmoe(5m)CpsmoeApsmoeGpsmoeApsGpsGpsTpsGpsTpsApsGps(5m) |
| | | CpsGpsApslnApsmoeGpsmoeTpsmoeGpsmoe(5m)C |
| ASO-1053 | 1074 | moeGpsmoe(5m)CpsmoeApsmoeGpsmoeApsGpsGpsTpsGpsTpsApsGps(5m) |
| | | CpsGpsApsmoeApslnGpsmoeTpsmoeGpsmoe(5m)C |
| ASO-1054 | 1075 | moeGpsmoe(5m)CpsmoeApsmoeGpsmoeApsGpsGpsTpsGpsTpsApsGps(5m) |
| | | CpsGpsApsmoeApsmoeGpslnTpsmoeGpsmoe(5m)C |
| ASO-1055 | 1076 | moeGpsmoe(5m)CpsmoeApsmoeGpsmoeApsGpsGpsTpsGpsTpsApsGps(5m) |
| | | CpsGpsApsmoeApsmoeGpsmoeTpslnGpsmoe(5m)C |
| ASO-1056 | 1077 | moeGpsmoe(5m)CpsmoeApsmoeGpsmoeApsGpsGpsTpsGpsTpsApsGps(5m) |
| | | CpsGpsApsmoeApsmoeGpsmoeTpsmoeGpsln(5m)C |
| ASO-1057 | 1078 | monGalNAc4-p-moeGpsmoe(5m)CpsmoeApsmoeGpsmoeApsGpsGpsTpsGps |
| | | ApsApsGps(5m)CpsGpsApsmoeApsmoeGpsmoeTpsmoeGpsmoe(5m)C |
| ASO-1058 | 1079 | monGalNAc4-p-moeGpsmoe(5m)CpsmoeApsmoeGpsApsGpsGpsTpsGpsmoe |
| | | ApsApsGps(5m)CpsGpsApsmoeApslnGpsmoeTpslnGpsln(5m)C |
| ASO-1059 | 1080 | monGalNAc4-p-moe(5m)CpslnApsmoeGpsmoeApsmoeGpsGpsTpsGpsAps |
| | | ApsGps(5m)CpsGpsApsApsmoeGpsmoeTpslnGpsmoe(5m)CpsmoeA |
| ASO-1060 | 1081 | moeGpsmoe(5m)CpsmoeApsmoeGpsmoeApsGpsGpsTpslm(5m)CpsApsAps |
| | | Gps(5m)CpsGpsApsmoeApsmoeGpsmoeTpsmoeGpsmoe(5m)C |
| ASO-1061 | 1082 | moeGpsmoe(5m)CpsmoeApsmoeGpsmoeApsGpsGpsTpslmTpsApsApsGps |
| | | (5m)CpsGpsApsmoeApsmoeGpsmoeTpsmoeGpsmoe(5m)C |
| ASO-1062 | 1083 | moeGpsmoe(5m)CpsmoeApsmoeGpsmoeApsGpsGpsTpsGpslmGpsApsGps |
| | | (5m)CpsGpsApsmoeApsmoeGpsmoeTpsmoeGpsmoe(5m)C |
| ASO-1063 | 1084 | moeGpsmoe(5m)CpsmoeApsmoeGpsmoeApsGpsGpsTpsGpsApslmTpsGps |
| | | (5m)CpsGpsApsmoeApsmoeGpsmoeTpsmoeGpsmoe(5m)C |
| ASO-1064 | 1085 | moeGpsmoe(5m)CpsmoeApsmoeGpsmoeApsGpsGpsTpsGpsApsApslmAps |
| | | (5m)CpsGpsApsmoeApsmoeGpsmoeTpsmoeGpsmoe(5m)C |
| ASO-1065 | 1086 | moeGpsmoe(5m)CpsmoeApsmoeGpsmoeApsGpsGpsTpsGpsApsApslm(5m) |
| | | Cps(5m)CpsGpsApsmoeApsmoeGpsmoeTpsmoeGpsmoe(5m)C |
| ASO-1066 | 1087 | moeGpsmoe(5m)CpsmoeApsmoeGpsmoeApsGpsGpsTpsGpsApsApslmTps |
| | | (5m)CpsGpsApsmoeApsmoeGpsmoeTpsmoeGpsmoe(5m)C |
| ASO-1067 | 1088 | monGalNAc4-p-moeGpsmoe(5m)CpsmoeApslnGpsmoeApsGpsGpsTpsGps |
| | | ApsmXpsGps(5m)CpsGpsApslnApsmoeGpsmoeTpsmoeGpsmoe(5m)C |
| ASO-1068 | 1089 | lnGpsln(5m)CpsmoeApsmoeGpsmoeApsGpsGpsTpsGpsTpsApsGps(5m)Cps |
| | | GpsApsmoeApsmoeGpsmoeTpsmoeGpsmoe(5m)C |
| ASO-1069 | 1090 | lnGpsmoe(5m)CpslnApsmoeGpsmoeApsGpsGpsTpsGpsTpsApsGps(5m)Cps |
| | | GpsApsmoeApsmoeGpsmoeTpsmoeGpsmoe(5m)C |
| ASO-1070 | 1091 | lnGpsmoe(5m)CpsmoeApslnGpsmoeApsGpsGpsTpsGpsTpsApsGps(5m)Cps |
| | | GpsApsmoeApsmoeGpsmoeTpsmoeGpsmoe(5m)C |
| ASO-1071 | 1092 | lnGpsmoe(5m)CpsmoeApsmoeGpslnApsGpsGpsTpsGpsTpsApsGps(5m)Cps |
| | | GpsApsmoeApsmoeGpsmoeTpsmoeGpsmoe(5m)C |
| ASO-1072 | 1093 | lnGpsmoe(5m)CpsmoeApsmoeGpsmoeApsGpsGpsTpsGpsTpsApsGps(5m)Cps |
| | | GpsApslnApsmoeGpsmoeTpsmoeGpsmoe(5m)C |
| ASO-1073 | 1094 | lnGpsmoe(5m)CpsmoeApsmoeGpsmoeApsGpsGpsTpsGpsTpsApsGps(5m)Cps |
| | | GpsApsmoeApslnGpsmoeTpsmoeGpsmoe(5m)C |
| ASO-1074 | 1095 | lnGpsmoe(5m)CpsmoeApsmoeGpsmoeApsGpsGpsTpsGpsTpsApsGps(5m)Cps |
| | | GpsApsmoeApsmoeGpslnTpsmoeGpsmoe(5m)C |
| ASO-1075 | 1096 | lnGpsmoe(5m)CpsmoeApsmoeGpsmoeApsGpsGpsTpsGpsTpsApsGps(5m)Cps |
| | | GpsApsmoeApsmoeGpsmoeTpslnGpsmoe(5m)C |
| ASO-1076 | 1097 | lnGpsmoe(5m)CpsmoeApsmoeGpsmoeApsGpsGpsTpsGpsTpsApsGps(5m)Cps |
| | | GpsApsmoeApsmoeGpsmoeTpsmoeGpsln(5m)C |
| ASO-1077 | 1098 | moeGpsln(5m)CpslnApsmoeGpsmoeApsGpsGpsTpsGpsTpsApsGps(5m)Cps |
| | | GpsApsmoeApsmoeGpsmoeTpsmoeGpsmoe(5m)C |
| ASO-1078 | 1099 | moeGpsln(5m)CpsmoeApslnGpsmoeApsGpsGpsTpsGpsTpsApsGps(5m)Cps |
| | | GpsApsmoeApsmoeGpsmoeTpsmoeGpsmoe(5m)C |
| ASO-1079 | 1100 | moeGpsln(5m)CpsmoeApsmoeGpslnApsGpsGpsTpsGpsTpsApsGps(5m)Cps |
| | | GpsApsmoeApsmoeGpsmoeTpsmoeGpsmoe(5m)C |
| ASO-1080 | 1101 | moeGpsln(5m)CpsmoeApsmoeGpsmoeApsGpsGpsTpsGpsTpsApsGps(5m)Cps |
| | | GpsApslnApsmoeGpsmoeTpsmoeGpsmoe(5m)C |
| ASO-1081 | 1102 | moeGpsln(5m)CpsmoeApsmoeGpsmoeApsGpsGpsTpsGpsTpsApsGps(5m)Cps |
| | | GpsApsmoeApslnGpsmoeTpsmoeGpsmoe(5m)C |
| ASO-1082 | 1103 | moeGpsln(5m)CpsmoeApsmoeGpsmoeApsGpsGpsTpsGpsTpsApsGps(5m)Cps |
| | | GpsApsmoeApsmoeGpslnTpsmoeGpsmoe(5m)C |
| ASO-1083 | 1104 | moeGpsln(5m)CpsmoeApsmoeGpsmoeApsGpsGpsTpsGpsTpsApsGps(5m)Cps |
| | | GpsApsmoeApsmoeGpsmoeTpslnGpsmoe(5m)C |
| ASO-1084 | 1105 | moeGpsln(5m)CpsmoeApsmoeGpsmoeApsGpsGpsTpsGpsTpsApsGps(5m)Cps |
| | | GpsApsmoeApsmoeGpsmoeTpsmoeGpsln(5m)C |
| ASO-1085 | 1106 | moeGpsmoe(5m)CpslnApslnGpsmoeApsGpsGpsTpsGpsTpsApsGps(5m)Cps |
| | | GpsApsmoeApsmoeGpsmoeTpsmoeGpsmoe(5m)C |
| ASO-1086 | 1107 | moeGpsmoe(5m)CpslnApsmoeGpslnApsGpsGpsTpsGpsTpsApsGps(5m)Cps |
| | | GpsApsmoeApsmoeGpsmoeTpsmoeGpsmoe(5m)C |
| ASO-1087 | 1108 | moeGpsmoe(5m)CpslnApsmoeGpsmoeApsGpsGpsTpsGpsTpsApsGps(5m)Cps |
| | | GpsApslnApsmoeGpsmoeTpsmoeGpsmoe(5m)C |
| ASO-1088 | 1109 | moeGpsmoe(5m)CpslnApsmoeGpsmoeApsGpsGpsTpsGpsTpsApsGps(5m)Cps |
| | | GpsApsmoeApslnGpsmoeTpsmoeGpsmoe(5m)C |
| ASO-1089 | 1110 | moeGpsmoe(5m)CpslnApsmoeGpsmoeApsGpsGpsTpsGpsTpsApsGps(5m)Cps |
| | | GpsApsmoeApsmoeGpslnTpsmoeGpsmoe(5m)C |
| ASO-1090 | 1111 | moeGpsmoe(5m)CpslnApsmoeGpsmoeApsGpsGpsTpsGpsTpsApsGps(5m)Cps |
| | | GpsApsmoeApsmoeGpsmoeTpslnGpsmoe(5m)C |
| ASO-1091 | 1112 | moeGpsmoe(5m)CpslnApsmoeGpsmoeApsGpsGpsTpsGpsTpsApsGps(5m)Cps |
| | | GpsApsmoeApsmoeGpsmoeTpsmoeGpsln(5m)C |
| ASO-1092 | 1113 | moeGpsmoe(5m)CpsmoeApsmoeGpslnApsGpsGpsTpsGpsTpsApsGps(5m)Cps |
| | | GpsApsmoeApsmoeGpsmoeTpslnGpsmoe(5m)C |
| ASO-1093 | 1114 | moeGpsmoe(5m)CpsmoeApsmoeGpslnApsGpsGpsTpsGpsTpsApsGps(5m)Cps |
| | | GpsApsmoeApsmoeGpslnTpsmoeGpsmoe(5m)C |
| ASO-1094 | 1115 | moeGpsmoe(5m)CpsmoeApsmoeGpslnApsGpsGpsTpsGpsTpsApsGps(5m)Cps |
| | | GpsApsmoeApslnGpsmoeTpsmoeGpsmoe(5m)C |
| ASO-1095 | 1116 | moeGpsmoe(5m)CpsmoeApsmoeGpslnApsGpsGpsTpsGpsTpsApsGps(5m)Cps |
| | | GpsApslnApsmoeGpsmoeTpsmoeGpsmoe(5m)C |
| ASO-1096 | 1117 | moeGpsmoe(5m)CpsmoeApslnGpsmoeApsGpsGpsTpsGpsTpsApsGps(5m)Cps |
| | | GpsApsmoeApsmoeGpsmoeTpsmoeGpsln(5m)C |
| ASO-1097 | 1118 | moeGpsmoe(5m)CpsmoeApslnGpsmoeApsGpsGpsTpsGpsTpsApsGps(5m)Cps |
| | | GpsApsmoeApsmoeGpsmoeTpslnGpsmoe(5m)C |
| ASO-1098 | 1119 | moeGpsmoe(5m)CpsmoeApslnGpsmoeApsGpsGpsTpsGpsTpsApsGps(5m)Cps |
| | | GpsApsmoeApsmoeGpslnTpsmoeGpsmoe(5m)C |
| ASO-1099 | 1120 | moeGpsmoe(5m)CpsmoeApslnGpsmoeApsGpsGpsTpsGpsTpsApsGps(5m)Cps |
| | | GpsApsmoeApslnGpsmoeTpsmoeGpsmoe(5m)C |
| ASO-1100 | 1121 | moeGpsmoe(5m)CpsmoeApslnGpsmoeApsGpsGpsTpsGpsTpsApsGps(5m)Cps |
| | | GpsApslnApsmoeGpsmoeTpsmoeGpsmoe(5m)C |
| ASO-1101 | 1122 | moeGpsmoe(5m)CpsmoeApslnGpslnApsGpsGpsTpsGpsTpsApsGps(5m)Cps |
| | | GpsApsmoeApsmoeGpsmoeTpsmoeGpsmoe(5m)C |
| ASO-1102 | 1123 | moeGpsmoe(5m)CpsmoeApsmoeGpsmoeApsGpsGpsTpsGpsTpsApsGps(5m) |
| | | CpsGpsApsmoeApsmoeGpsmoeTpslnGpsln(5m)C |
| ASO-1103 | 1124 | moeGpsmoe(5m)CpsmoeApsmoeGpsmoeApsGpsGpsTpsGpsTpsApsGps(5m) |
| | | CpsGpsApsmoeApsmoeGpslnTpsmoeGpsln(5m)C |
| ASO-1104 | 1125 | moeGpsmoe(5m)CpsmoeApsmoeGpsmoeApsGpsGpsTpsGpsTpsApsGps(5m) |
| | | CpsGpsApsmoeApsmoeGpslnTpslnGpsmoe(5m)C |
| ASO-1105 | 1126 | moeGpsmoe(5m)CpsmoeApsmoeGpsmoeApsGpsGpsTpsGpsTpsApsGps(5m) |
| | | CpsGpsApsmoeApslnGpsmoeTpsmoeGpsln(5m)C |
| ASO-1106 | 1127 | moeGpsmoe(5m)CpsmoeApsmoeGpsmoeApsGpsGpsTpsGpsTpsApsGps(5m) |
| | | CpsGpsApsmoeApslnGpsmoeTpslnGpsmoe(5m)C |
| ASO-1107 | 1128 | moeGpsmoe(5m)CpsmoeApsmoeGpsmoeApsGpsGpsTpsGpsTpsApsGps(5m) |
| | | CpsGpsApsmoeApslnGpslnTpsmoeGpsmoe(5m)C |
| ASO-1108 | 1129 | moeGpsmoe(5m)CpsmoeApsmoeGpsmoeApsGpsGpsTpsGpsTpsApsGps(5m) |
| | | CpsGpsApslnApsmoeGpsmoeTpsmoeGpsln(5m)C |
| ASO-1109 | 1130 | moeGpsmoe(5m)CpsmoeApsmoeGpsmoeApsGpsGpsTpsGpsTpsApsGps(5m) |
| | | CpsGpsApslnApsmoeGpsmoeTpslnGpsmoe(5m)C |
| ASO-1110 | 1131 | moeGpsmoe(5m)CpsmoeApsmoeGpsmoeApsGpsGpsTpsGpsTpsApsGps(5m) |
| | | CpsGpsApslnApsmoeGpslnTpsmoeGpsmoe(5m)C |
| ASO-1111 | 1132 | moeGpsmoe(5m)CpsmoeApsmoeGpsmoeApsGpsGpsTpsGpsTpsApsGps(5m) |
| | | CpsGpsApslnApslnGpsmoeTpsmoeGpsmoe(5m)C |
| ASO-1112 | 1133 | moeGpsmoe(5m)CpsmoeApsmoeGpslnApsGpsGpsTpsGpsTpsApsGps(5m) |
| | | CpsGpsApsmoeApsmoeGpsmoeTpsmoeGpsln(5m)C |
| ASO-1113 | 1134 | gutbGpsmoe(5m)CpsmoeApsmoeGpsmoeApsGpsGpsTpsGpsApsmXpsGps |
| | | (5m)CpsGpsApsmoeApsmoeGpsmoeTpsmoeGpsmoe(5m)C |
| ASO-1114 | 1135 | moeGpsgutb(5m)CpsmoeApsmoeGpsmoeApsGpsGpsTpsGpsApsmXpsGps |
| | | (5m)CpsGpsApsmoeApsmoeGpsmoeTpsmoeGpsmoe(5m)C |
| ASO-1115 | 1136 | moeGpsmoe(5m)CpsgutbApsmoeGpsmoeApsGpsGpsTpsGpsApsmXpsGps |
| | | (5m)CpsGpsApsmoeApsmoeGpsmoeTpsmoeGpsmoe(5m)C |
| ASO-1116 | 1137 | moeGpsmoe(5m)CpsmoeApsgutbGpsmoeApsGpsGpsTpsGpsApsmXpsGps |
| | | (5m)CpsGpsApsmoeApsmoeGpsmoeTpsmoeGpsmoe(5m)C |
| ASO-1117 | 1138 | moeGpsmoe(5m)CpsmoeApsmoeGpsgutbApsGpsGpsTpsGpsApsmXpsGps |
| | | (5m)CpsGpsApsmoeApsmoeGpsmoeTpsmoeGpsmoe(5m)C |
| ASO-1118 | 1139 | moeGpsmoe(5m)CpsmoeApsmoeGpsmoeApsGpsGpsTpsGpsApsmXpsGps |
| | | (5m)CpsGpsApsgutbApsmoeGpsmoeTpsmoeGpsmoe(5m)C |
| ASO-1119 | 1140 | moeGpsmoe(5m)CpsmoeApsmoeGpsmoeApsGpsGpsTpsGpsApsmXpsGps |
| | | (5m)CpsGpsApsmoeApsgutbGpsmoeTpsmoeGpsmoe(5m)C |
| ASO-1120 | 1141 | moeGpsmoe(5m)CpsmoeApsmoeGpsmoeApsGpsGpsTpsGpsApsmXpsGps |
| | | (5m)CpsGpsApsmoeApsmoeGpsgutbTpsmoeGpsmoe(5m)C |
| ASO-1121 | 1142 | moeGpsmoe(5m)CpsmoeApsmoeGpsmoeApsGpsGpsTpsGpsApsmXpsGps |
| | | (5m)CpsGpsApsmoeApsmoeGpsmoeTpsgutbGpsmoe(5m)C |
| ASO-1122 | 1143 | moeGpsmoe(5m)CpsmoeApsmoeGpsmoeApsGpsGpsTpsGpsApsmXpsGps |
| | | (5m)CpsGpsApsmoeApsmoeGpsmoeTpsmoeGpsgutb(5m)C |
| ASO-1123 | 1144 | gutbGpsmoe(5m)CpsmoeApsmoeGpsmoeApsGpsGpsTpsGpsTpsApsGps(5m) |
| | | CpsGpsApsmoeApsmoeGpsmoeTpsmoeGpsmoe(5m)C |
| ASO-1124 | 1145 | moeGpsgutb(5m)CpsmoeApsmoeGpsmoeApsGpsGpsTpsGpsTpsApsGps(5m) |
| | | CpsGpsApsmoeApsmoeGpsmoeTpsmoeGpsmoe(5m)C |
| ASO-1125 | 1146 | moeGpsmoe(5m)CpsgutbApsmoeGpsmoeApsGpsGpsTpsGpsTpsApsGps(5m) |
| | | CpsGpsApsmoeApsmoeGpsmoeTpsmoeGpsmoe(5m)C |
| ASO-1126 | 1147 | moeGpsmoe(5m)CpsmoeApsgutbGpsmoeApsGpsGpsTpsGpsTpsApsGps(5m) |
| | | CpsGpsApsmoeApsmoeGpsmoeTpsmoeGpsmoe(5m)C |
| ASO-1127 | 1148 | moeGpsmoe(5m)CpsmoeApsmoeGpsgutbApsGpsGpsTpsGpsTpsApsGps(5m) |
| | | CpsGpsApsmoeApsmoeGpsmoeTpsmoeGpsmoe(5m)C |
| ASO-1128 | 1149 | moeGpsmoe(5m)CpsmoeApsmoeGpsmoeApsGpsGpsTpsGpsTpsApsGps(5m) |
| | | CpsGpsApsgutbApsmoeGpsmoeTpsmoeGpsmoe(5m)C |
| ASO-1129 | 1150 | moeGpsmoe(5m)CpsmoeApsmoeGpsmoeApsGpsGpsTpsGpsTpsApsGps(5m) |
| | | CpsGpsApsmoeApsgutbGpsmoeTpsmoeGpsmoe(5m)C |
| ASO-1130 | 1151 | moeGpsmoe(5m)CpsmoeApsmoeGpsmoeApsGpsGpsTpsGpsTpsApsGps(5m) |
| | | CpsGpsApsmoeApsmoeGpsgutbTpsmoeGpsmoe(5m)C |
| ASO-1131 | 1152 | moeGpsmoe(5m)CpsmoeApsmoeGpsmoeApsGpsGpsTpsGpsTpsApsGps(5m) |
| | | CpsGpsApsmoeApsmoeGpsmoeTpsgutbGpsmoe(5m)C |
| ASO-1132 | 1153 | moeGpsmoe(5m)CpsmoeApsmoeGpsmoeApsGpsGpsTpsGpsTpsApsGps(5m) |
| | | CpsGpsApsmoeApsmoeGpsmoeTpsmoeGpsgutb(5m)C |
| ASO-1133 | 1154 | monGalNAc4-p-moeGpsmoe(5m)CpsmoeApsmoeGpslnApsGpsGpsTpsGps |
| | | ApsmXpsGps(5m)CpsGpsApslnApsmoeGpsmoeTpsmoeGpsmoe(5m)C |
| ASO-1134 | 1155 | monGalNAc4-p-moeGpsmoe(5m)CpsmoeApsmoeGpsmoeApsGpsGpsTpsGps |
| | | ApsmXpsGps(5m)CpsGpsApslnApsmoeGpslnTpsmoeGpsmoe(5m)C |
| ASO-1135 | 1156 | moe(5m)CpsmoeApsmGpsmoeApsmGpsGpsTpsGpsApsApsGps(5m)CpsGps |
| | | ApsApsmoeGpsmoeTpsgutbGpsmoe(5m)CpsmoeA |
| ASO-1136 | 1157 | moe(5m)CpsmoeApsmoeGpsmoeApsmoeGpsGpsTpsGpsApsApsGps(5m)Cps |
| | | GpsApsApsmoeGpsmoeTpsgutbGpsm(5m)CpsmA |
| ASO-1137 | 1158 | moe(5m)CpsmoeApsmoeGpsmoeApsmoeGpsGpsTpsGpsApsApsGps(5m)Cps |
| | | GpsApsApsmGpsmoeTpsgutbGpsmoe(5m)CpsmA |
| ASO-1138 | 1159 | moe(5m)CpsmoeApsmoeGpsmoeApsmoeGpsGpsTpsGpsApsApsGps(5m)Cps |
| | | GpsApsApslnGpsmoeTpsgutbGpsmoe(5m)CpsmoeA |
| ASO-1139 | 1160 | moe(5m)CpsmoeApsmoeGpsmoeApsmoeGpsGpsTpsGpsApsApsGps(5m)Cps |
| | | GpsApsApsmoeGpslnTpsgutbGpsmoe(5m)CpsmoeA |
| ASO-1140 | 1161 | moe(5m)CpsmoeApsmoeGpsmoeApsmoeGpsGpsTpsGpsApsApsGps(5m)Cps |
| | | GpsApsApsmoeGpsmoeTpsgutbGpsln(5m)CpsmoeA |
| ASO-1141 | 1162 | moe(5m)CpsmoeApsmoeGpsmoeApsmoeGpsGpsTpsGpsApsApsGps(5m)Cps |
| | | GpsApsApsmoeGpsmoeTpsgutbGpsmoe(5m)CpslnA |
| ASO-1142 | 1163 | moe(5m)CpsmoeApslnGpsmoeApslnGpsGpsTpsGpsApsApsGps(5m)CpsGps |
| | | ApsApsmoeGpsmoeTpsgutbGpsmoe(5m)CpsmoeA |
| ASO-1143 | 1164 | moe(5m)CpsmoeApslnGpsmoeApsmoeGpsGpsTpsGpsApsApsGps(5m)Cps |
| | | GpsApsApslnGpsmoeTpsgutbGpsmoe(5m)CpsmoeA |
| ASO-1144 | 1165 | moe(5m)CpsmoeApsmoeGpsmoeApslnGpsGpsTpsGpsApsApsGps(5m)Cps |
| | | GpsApsApslnGpsmoeTpsgutbGpsmoe(5m)CpsmoeA |
| ASO-1145 | 1166 | moe(5m)CpsmoeApsmoeGpsmoeApsmoeGpsGpsTpsGpsApsApsGps(5m)Cps |
| | | GpsApsApslnGpsmoeTpsgutbGpsmoe(5m)CpslnA |
| ASO-1146 | 1167 | moe(5m)CpslnApsmoeGpsmoeApsmoeGpsGpsTpsGpsApsApsGps(5m)Cps |
| | | GpsApsApsmoeGpslnTpsgutbGpsmoe(5m)CpsmoeA |
| ASO-1147 | 1168 | moe(5m)CpsmoeApsmGpsmoeApsmGpsGpsTpsGpsApsApsGps(5m)CpsGps |
| | | ApsApsmoeGpsmoeTpsmoeGpsgutb(5m)CpsmoeA |
| ASO-1148 | 1169 | moe(5m)CpsmoeApsmoeGpsmoeApsmoeGpsGpsTpsGpsApsApsGps(5m)Cps |
| | | GpsApsApsmoeGpsmoeTpsmGpsgutb(5m)CpsmA |
| ASO-1149 | 1170 | moe(5m)CpsmoeApsmoeGpsmoeApsmoeGpsGpsTpsGpsApsApsGps(5m)Cps |
| | | GpsApsApsmGpsmoeTpsmGpsgutb(5m)CpsmoeA |
| ASO-1150 | 1171 | moe(5m)CpsmoeApsmoeGpsmoeApsmoeGpsGpsTpsGpsApsApsGps(5m)Cps |
| | | GpsApsApslnGpsmoeTpsmoeGpsgutb(5m)CpsmoeA |
| ASO-1151 | 1172 | moe(5m)CpsmoeApsmoeGpsmoeApsmoeGpsGpsTpsGpsApsApsGps(5m)Cps |
| | | GpsApsApsmoeGpslnTpsmoeGpsgutb(5m)CpsmoeA |
| ASO-1152 | 1173 | moe(5m)CpsmoeApsmoeGpsmoeApsmoeGpsGpsTpsGpsApsApsGps(5m)Cps |
| | | GpsApsApsmoeGpsmoeTpslnGpsgutb(5m)CpsmoeA |
| ASO-1153 | 1174 | moe(5m)CpsmoeApsmoeGpsmoeApsmoeGpsGpsTpsGpsApsApsGps(5m)Cps |
| | | GpsApsApsmoeGpsmoeTpsmoeGpsgutb(5m)CpslnA |
| ASO-1154 | 1175 | moe(5m)CpsmoeApslnGpsmoeApslnGpsGpsTpsGpsApsApsGps(5m)CpsGps |
| | | ApsApsmoeGpsmoeTpsmoeGpsgutb(5m)CpsmoeA |
| ASO-1155 | 1176 | moe(5m)CpsmoeApslnGpsmoeApsmoeGpsGpsTpsGpsApsApsGps(5m)Cps |
| | | GpsApsApslnGpsmoeTpsmoeGpsgutb(5m)CpsmoeA |
| ASO-1156 | 1177 | moe(5m)CpsmoeApsmoeGpsmoeApslnGpsGpsTpsGpsApsApsGps(5m)Cps |
| | | GpsApsApslnGpsmoeTpsmoeGpsgutb(5m)CpsmoeA |
| ASO-1157 | 1178 | moe(5m)CpsmoeApsmoeGpsmoeApsmoeGpsGpsTpsGpsApsApsGps(5m)Cps |
| | | GpsApsApslnGpsmoeTpsmoeGpsgutb(5m)CpslnA |
| ASO-1158 | 1179 | moe(5m)CpslnApsmoeGpsmoeApsmoeGpsGpsTpsGpsApsApsGps(5m)Cps |
| | | GpsApsApsmoeGpslnTpsmoeGpsgutb(5m)CpsmoeA |
| ASO-1159 | 1180 | moe(5m)CpslnApsmoeGpsmoeApsmoeGpsGpsTpsGpsApsApsGps(5m)Cps |
| | | GpsApsApsmoeGpsmoeTpslnGpsgutb(5m)CpsmoeA |
| ASO-1160 | 1181 | moeGpsmoe(5m)CpslnApsmoeGpslnApsGpsGpsTpsGpsApsApsAps(5m)Cps |
| | | GpsApsmoeApsmoeGpsmoeTpsmoeGpsmoe(5m)C |
| ASO-1161 | 1182 | moeGpsmoe(5m)CpsmoeApslnGpslnApsGpsGpsTpsGpsApsApsAps(5m)Cps |
| | | GpsApsmoeApsmoeGpsmoeTpsmoeGpsmoe(5m)C |
| ASO-1162 | 1183 | moeGpsmoe(5m)CpsmoeApslnGpsmoeApsGpsGpsTpsGpsApsApsAps(5m) |
| | | CpsGpsApslnApsmoeGpsmoeTpsmoeGpsmoe(5m)C |
| ASO-1163 | 1184 | moeGpsmoe(5m)CpsmoeApsmoeGpslnApsGpsGpsTpsGpsApsApsAps(5m) |
| | | CpsGpsApslnApsmoeGpsmoeTpsmoeGpsmoe(5m)C |
| ASO-1164 | 1185 | moeGpsmoe(5m)CpsmoeApsmoeGpslnApsGpsGpsTpsGpsApsApsAps(5m) |
| | | CpsGpsApsmoeApsmoeGpsmoeTpslnGpsmoe(5m)C |
| ASO-1165 | 1186 | moeGpsmoe(5m)CpsmoeApsmoeGpsmoeApsGpsGpsTpsGpsApsApsAps(5m) |
| | | CpsGpsApslnApsmoeGpslnTpsmoeGpsmoe(5m)C |
| ASO-1166 | 1187 | monGalNAc4-p-moeGpsmoe(5m)CpsmoeApsmoeGpsmoeApsGpsGpsTpsGps |
| | | ApsmXpsGps(5m)CpsGpsApsmoeApsmoeGpsmoeTpsmoeGpsmoe(5m)C |
| ASO-1167 | 1188 | moeGpsmoe(5m)CpslnApsmoeGpslnApsGpsGpsTpsGpsApsdXpsGps(5m)Cps |
| | | GpsApsmoeApsmoeGpsmoeTpsmoeGpsmoe(5m)C |
| ASO-1168 | 1189 | moeGpsmoe(5m)CpsmoeApslnGpslnApsGpsGpsTpsGpsApsdXpsGps(5m)Cps |
| | | GpsApsmoeApsmoeGpsmoeTpsmoeGpsmoe(5m)C |
| ASO-1169 | 1190 | moeGpsmoe(5m)CpsmoeApslnGpsmoeApsGpsGpsTpsGpsApsdXpsGps(5m) |
| | | CpsGpsApslnApsmoeGpsmoeTpsmoeGpsmoe(5m)C |
| ASO-1170 | 1191 | moeGpsmoe(5m)CpsmoeApsmoeGpslnApsGpsGpsTpsGpsApsdXpsGps(5m) |
| | | CpsGpsApslnApsmoeGpsmoeTpsmoeGpsmoe(5m)C |
| ASO-1171 | 1192 | moeGpsmoe(5m)CpsmoeApsmoeGpslnApsGpsGpsTpsGpsApsdXpsGps(5m) |
| | | CpsGpsApsmoeApsmoeGpsmoeTpslnGpsmoe(5m)C |
| ASO-1172 | 1193 | moeGpsmoe(5m)CpsmoeApsmoeGpsmoeApsGpsGpsTpsGpsApsdXpsGps |
| | | (5m)CpsGpsApslnApsmoeGpslnTpsmoeGpsmoe(5m)C |
| ASO-1173 | 1194 | moeGpsmoe(5m)CpsmoeApsmoeGpsmoeApsGpsGpsTpslmCpsApsApsGps |
| | | (5m)CpsGpsApsmoeApsmoeGpsmoeTpsmoeGpsmoe(5m)C |
| ASO-1174 | 1195 | moeGpsmoe(5m)CpsmoeApsmoeGpsmoeApsGpsGpsTpslmUpsApsApsGps |
| | | (5m)CpsGpsApsmoeApsmoeGpsmoeTpsmoeGpsmoe(5m)C |
| ASO-1175 | 1196 | moeGpsmoe(5m)CpsmoeApsmoeGpsmoeApsGpsGpsTpsGpslmGpsApsGps |
| | | (5m)CpsGpsApsmoeApsmoeGpsmoeTpsmoeGpsmoe(5m)C |
| ASO-1176 | 1197 | moeGpsmoe(5m)CpsmoeApsmoeGpsmoeApsGpsGpsTpsGpsApslmUpsGps |
| | | (5m)CpsGpsApsmoeApsmoeGpsmoeTpsmoeGpsmoe(5m)C |
| ASO-1177 | 1198 | moeGpsmoe(5m)CpsmoeApsmoeGpsmoeApsGpsGpsTpsGpsApsApslmCps |
| | | (5m)CpsGpsApsmoeApsmoeGpsmoeTpsmoeGpsmoe(5m)C |
| ASO-1178 | 1199 | moeGpsmoe(5m)CpsmoeApsmoeGpsmoeApsGpsGpsTpsGpsApsApslmUps |
| | | (5m)CpsGpsApsmoeApsmoeGpsmoeTpsmoeGpsmoe(5m)C |
| ASO-1179 | 1200 | monGalNAc4-p-moeGpsmoe(5m)CpsmoeApsmoeGpsmoeApsGpsGpsTpsGps |
| | | ApsmXpsGps(5m)CpsGpsApsmoeApsmoeGpslnTpsmoeGpsln(5m)C |
| ASO-1180 | 1201 | monGalNAc4-p-moeGpsmoe(5m)CpsmoeApsmoeGpsmoeApsGpsGpsTpsGps |
| | | ApsApsTps(5m)CpsGpsApslnApsmoeGpslnTpsmoeGpsmoe(5m)C |
| ASO-1181 | 1202 | monGalNAc4-p-moeGpsmoe(5m)CpsmoeApsmoeGpsmoeApsGpsGpsTpsGps |
| | | ApsmXpsGps(5m)CpsGpsApsmoeApsmoeGpslnTpslnGpsmoe(5m)C |
| ASO-1182 | 1203 | moe(5m)CpsmoeApsmoeGpsmoeApsmoeGpsGpsTpsGpsApsApsGps(5m)Cps |
| | | GpsApsApsmGpss?moeTpsmGpsmoe(5m)CpsmA |
| ASO-1183 | 1204 | moe(5m)CpsmoeApsmoeGpsmoeApsmoeGpsGpsTpsGpsApsApsGps(5m)Cps |
| | | GpsApsApsmGpsr?moeTpsmGpsmoe(5m)CpsmA |
| ASO-1184 | 1205 | moe(5m)CpsmoeApsmoeGpsmoeApsmoeGpsGpsTpsGpsApsApsGps(5m)Cps |
| | | GpsApsApsmGpsmoeTpsmGpss?moe(5m)CpsmA |
| ASO-1185 | 1206 | moe(5m)CpsmoeApsmoeGpsmoeApsmoeGpsGpsTpsGpsApsApsGps(5m)Cps |
| | | GpsApsApsmGpsmoeTpsmGpsr?moe(5m)CpsmA |
| ASO-1186 | 1207 | moe(5m)CpsmoeApsmoeGpsmoeApsmoeGpsGpsTpsGpsApsApsGps(5m)Cps |
| | | GpsApsApsmGpss?moeTpsmGpss?moe(5m)CpsmA |
| ASO-1187 | 1208 | moe(5m)CpsmoeApsmoeGpsmoeApsmoeGpsGpsTpsGpsApsApsGps(5m)Cps |
| | | GpsApsApsmGpsr?moeTpsmGpsr?moe(5m)CpsmA |
| ASO-1188 | 1209 | moe(5m)CpsmoeApsmoeGpsmoeApsmoeGpsGpsTpsGpsApsApsGps(5m)Cps |
| | | GpsApsApsmGpss?moeTpsmGpsr?moe(5m)CpsmA |
| ASO-1189 | 1210 | moe(5m)CpsmoeApsmoeGpsmoeApsmoeGpsGpsTpsGpsApsApsGps(5m)Cps |
| | | GpsApsApsmGpsr?moeTpsmGpss?moe(5m)CpsmA |
| ASO-1190 | 1211 | moeGpsmoeCpsmoeApsmoeGpsApsGpsGpsTpsGpsmoeApsApsGpsdCpsGps |
| | | ApsmoeApslnApsmoeTpslnGpslnC |
| ASO-1191 | 1212 | moeGpsmoe(5m)CpsmoeApsmoeGpslnApsGpsGpsTpsGpsApsdXpsGps(5m) |
| | | CpsGpsApsmoeApsmoeGpslnTpsmoeGpsmoe(5m)C |
| ASO-1192 | 1213 | moeGpsmoe(5m)CpsmoeApsmoeGpsmoeApsGpsGpsTpsGpsApsdXpsGps |
| | | (5m)CpsGpsApsmoeApsmoeGpslnTpslnGpsmoe(5m)C |
| ASO-1193 | 1214 | moeGpsmoe(5m)CpsmoeApsmoeGpsmoeApsGpsGpsTpsGpsApsdXpsGps |
| | | (5m)CpsGpsApsmoeApsmoeGpslnTpsmoeGpsln(5m)C |
| ASO-1194 | 1215 | monGalNAc4-p-moeGpsmoe(5m)CpslnApsmoeGpsmoeApsGpsGpsTpsGps |
| | | ApsmXpsGps(5m)CpsGpsApsmoeApsmoeGpslnTpsmoeGpsmoe(5m)C |
| ASO-1195 | 1216 | monGalNAc4-p-moeGpsmoe(5m)CpsmoeApsmoeGpsmoeApsGpsGpsTpsGps |
| | | TpsApsGps(5m)CpsGpsApsmoeApsmoeGpsmoeTpsmoeGpsmoe(5m)C |
| ASO-1196 | 1217 | moeGpsln(5m)CpsmoeApsmoeGpsmoeApsGpsGpsTpsGpsApsdXpsGps(5m) |
| | | CpsGpsApslnApsmoeGpsmoeTpsmoeGpsmoe(5m)C |
| ASO-1197 | 1218 | moeGpsmoe(5m)CpslnApsmoeGpsmoeApsGpsGpsTpsGpsApsdXpsGps(5m) |
| | | CpsGpsApsmoeApsmoeGpslnTpsmoeGpsmoe(5m)C |
| ASO-1198 | 1219 | moeGpsmoe(5m)CpsmoeApsmoeGpsmoeApsGpsGpsTpsGpsApsdXpsGps |
| | | (5m)CpsGpsApslnApsmoeGpsmoeTpslnGpsmoe(5m)C |
| ASO-1199 | 1220 | moeGpsmoe(5m)CpsmoeApslnGpsmoeApsGpsGpsTpsGpsApsdXpsGps(5m) |
| | | CpsGpsApsmoeApsmoeGpslnTpsmoeGpsmoe(5m)C |
| ASO-1200 | 1221 | lnGpsmoe(5m)CpsmoeApsmoeGpsmoeApsGpsGpsTpsGpsApsdXpsGps(5m) |
| | | CpsGpsApsmoeApsmoeGpsmoeTpsmoeGpsmoe(5m)C |
| ASO-1201 | 1222 | moeGpsln(5m)CpsmoeApsmoeGpsmoeApsGpsGpsTpsGpsApsdXpsGps(5m) |
| | | CpsGpsApsmoeApsmoeGpsmoeTpsmoeGpsmoe(5m)C |
| ASO-1202 | 1223 | moeGpsmoe(5m)CpslnApsmoeGpsmoeApsGpsGpsTpsGpsApsdXpsGps(5m) |
| | | CpsGpsApsmoeApsmoeGpsmoeTpsmoeGpsmoe(5m)C |
| ASO-1203 | 1224 | moeGpsmoe(5m)CpsmoeApslnGpsmoeApsGpsGpsTpsGpsApsdXpsGps(5m) |
| | | CpsGpsApsmoeApsmoeGpsmoeTpsmoeGpsmoe(5m)C |
| ASO-1204 | 1225 | moeGpsmoe(5m)CpsmoeApsmoeGpslnApsGpsGpsTpsGpsApsdXpsGps(5m) |
| | | CpsGpsApsmoeApsmoeGpsmoeTpsmoeGpsmoe(5m)C |
| ASO-1205 | 1226 | moeGpsmoe(5m)CpsmoeApsmoeGpsmoeApsGpsGpsTpsGpsApsdXpsGps |
| | | (5m)CpsGpsApslnApsmoeGpsmoeTpsmoeGpsmoe(5m)C |
| ASO-1206 | 1227 | moeGpsmoe(5m)CpsmoeApsmoeGpsmoeApsGpsGpsTpsGpsApsdXpsGps |
| | | (5m)CpsGpsApsmoeApslnGpsmoeTpsmoeGpsmoe(5m)C |
| ASO-1207 | 1228 | moeGpsmoe(5m)CpsmoeApsmoeGpsmoeApsGpsGpsTpsGpsApsdXpsGps |
| | | (5m)CpsGpsApsmoeApsmoeGpslnTpsmoeGpsmoe(5m)C |
| ASO-1208 | 1229 | moeGpsmoe(5m)CpsmoeApsmoeGpsmoeApsGpsGpsTpsGpsApsdXpsGps |
| | | (5m)CpsGpsApsmoeApsmoeGpsmoeTpslnGpsmoe(5m)C |
| ASO-1209 | 1230 | moeGpsmoe(5m)CpsmoeApsmoeGpsmoeApsGpsGpsTpsGpsApsdXpsGps |
| | | (5m)CpsGpsApsmoeApsmoeGpsmoeTpsmoeGpsln(5m)C |
| ASO-1210 | 1231 | lnGpsln(5m)CpsmoeApsmoeGpsmoeApsGpsGpsTpsGpsApsdXpsGps(5m)Cps |
| | | GpsApsmoeApsmoeGpsmoeTpsmoeGpsmoe(5m)C |
| ASO-1211 | 1232 | lnGpsmoe(5m)CpslnApsmoeGpsmoeApsGpsGpsTpsGpsApsdXpsGps(5m)Cps |
| | | GpsApsmoeApsmoeGpsmoeTpsmoeGpsmoe(5m)C |
| ASO-1212 | 1233 | lnGpsmoe(5m)CpsmoeApslnGpsmoeApsGpsGpsTpsGpsApsdXpsGps(5m)Cps |
| | | GpsApsmoeApsmoeGpsmoeTpsmoeGpsmoe(5m)C |
| ASO-1213 | 1234 | lnGpsmoe(5m)CpsmoeApsmoeGpslnApsGpsGpsTpsGpsApsdXpsGps(5m)Cps |
| | | GpsApsmoeApsmoeGpsmoeTpsmoeGpsmoe(5m)C |
| ASO-1214 | 1235 | lnGpsmoe(5m)CpsmoeApsmoeGpsmoeApsGpsGpsTpsGpsApsdXpsGps(5m) |
| | | CpsGpsApslnApsmoeGpsmoeTpsmoeGpsmoe(5m)C |
| ASO-1215 | 1236 | lnGpsmoe(5m)CpsmoeApsmoeGpsmoeApsGpsGpsTpsGpsApsdXpsGps(5m) |
| | | CpsGpsApsmoeApslnGpsmoeTpsmoeGpsmoe(5m)C |
| ASO-1216 | 1237 | lnGpsmoe(5m)CpsmoeApsmoeGpsmoeApsGpsGpsTpsGpsApsdXpsGps(5m) |
| | | CpsGpsApsmoeApsmoeGpslnTpsmoeGpsmoe(5m)C |
| ASO-1217 | 1238 | lnGpsmoe(5m)CpsmoeApsmoeGpsmoeApsGpsGpsTpsGpsApsdXpsGps(5m) |
| | | CpsGpsApsmoeApsmoeGpsmoeTpslnGpsmoe(5m)C |
| ASO-1218 | 1239 | lnGpsmoe(5m)CpsmoeApsmoeGpsmoeApsGpsGpsTpsGpsApsdXpsGps(5m) |
| | | CpsGpsApsmoeApsmoeGpsmoeTpsmoeGpsln(5m)C |
| ASO-1219 | 1240 | moeGpsln(5m)CpslnApsmoeGpsmoeApsGpsGpsTpsGpsApsdXpsGps(5m)Cps |
| | | GpsApsmoeApsmoeGpsmoeTpsmoeGpsmoe(5m)C |
| ASO-1220 | 1241 | moeGpsln(5m)CpsmoeApslnGpsmoeApsGpsGpsTpsGpsApsdXpsGps(5m)Cps |
| | | GpsApsmoeApsmoeGpsmoeTpsmoeGpsmoe(5m)C |
| ASO-1221 | 1242 | moeGpsln(5m)CpsmoeApsmoeGpslnApsGpsGpsTpsGpsApsdXpsGps(5m)Cps |
| | | GpsApsmoeApsmoeGpsmoeTpsmoeGpsmoe(5m)C |
| ASO-1222 | 1243 | moeGpsln(5m)CpsmoeApsmoeGpsmoeApsGpsGpsTpsGpsApsdXpsGps(5m) |
| | | CpsGpsApsmoeApslnGpsmoeTpsmoeGpsmoe(5m)C |
| ASO-1223 | 1244 | moeGpsln(5m)CpsmoeApsmoeGpsmoeApsGpsGpsTpsGpsApsdXpsGps(5m) |
| | | CpsGpsApsmoeApsmoeGpslnTpsmoeGpsmoe(5m)C |
| ASO-1224 | 1245 | moeGpsln(5m)CpsmoeApsmoeGpsmoeApsGpsGpsTpsGpsApsdXpsGps(5m) |
| | | CpsGpsApsmoeApsmoeGpsmoeTpslnGpsmoe(5m)C |
| ASO-1225 | 1246 | moeGpsln(5m)CpsmoeApsmoeGpsmoeApsGpsGpsTpsGpsApsdXpsGps(5m) |
| | | CpsGpsApsmoeApsmoeGpsmoeTpsmoeGpsln(5m)C |
| ASO-1226 | 1247 | moeGpsmoe(5m)CpslnApslnGpsmoeApsGpsGpsTpsGpsApsdXpsGps(5m)Cps |
| | | GpsApsmoeApsmoeGpsmoeTpsmoeGpsmoe(5m)C |
| ASO-1227 | 1248 | moeGpsmoe(5m)CpslnApsmoeGpsmoeApsGpsGpsTpsGpsApsdXpsGps(5m) |
| | | CpsGpsApslnApsmoeGpsmoeTpsmoeGpsmoe(5m)C |
| ASO-1228 | 1249 | moeGpsmoe(5m)CpslnApsmoeGpsmoeApsGpsGpsTpsGpsApsdXpsGps(5m) |
| | | CpsGpsApsmoeApslnGpsmoeTpsmoeGpsmoe(5m)C |
| ASO-1229 | 1250 | moeGpsmoe(5m)CpslnApsmoeGpsmoeApsGpsGpsTpsGpsApsdXpsGps(5m) |
| | | CpsGpsApsmoeApsmoeGpsmoeTpslnGpsmoe(5m)C |
| ASO-1230 | 1251 | moeGpsmoe(5m)CpslnApsmoeGpsmoeApsdGpsdGpsdTpsdGpsdApsdXpsd |
| | | Gpsd(5m)CpsdGpsdApsmoeApsmoeGpsmoeTpsmoeGpsln(5m)C |
| ASO-1231 | 1252 | moeGpsmoe(5m)CpsmoeApslnGpsmoeApsdGpsdGpsdTpsdGpsdApsdXpsd |
| | | Gpsd(5m)CpsdGpsdApsmoeApslnGpsmoeTpsmoeGpsmoe(5m)C |
| ASO-1232 | 1253 | moeGpsmoe(5m)CpsmoeApslnGpsmoeApsdGpsdGpsdTpsdGpsdApsdXpsd |
| | | Gpsd(5m)CpsdGpsdApsmoeApsmoeGpsmoeTpslnGpsmoe(5m)C |
| ASO-1233 | 1254 | moeGpsmoe(5m)CpsmoeApslnGpsmoeApsdGpsdGpsdTpsdGpsdApsdXpsd |
| | | Gpsd(5m)CpsdGpsdApsmoeApsmoeGpsmoeTpsmoeGpsln(5m)C |
| ASO-1234 | 1255 | moeGpsmoe(5m)CpsmoeApsmoeGpslnApsdGpsdGpsdTpsdGpsdApsdXpsd |
| | | Gpsd(5m)CpsdGpsdApsmoeApslnGpsmoeTpsmoeGpsmoe(5m)C |
| ASO-1235 | 1256 | moeGpsmoe(5m)CpsmoeApsmoeGpsmoeApsdGpsdGpsdTpsdGpsdApsdXpsd |
| | | Gpsd(5m)CpsdGpsdApslnApsmoeGpsmoeTpsmoeGpsln(5m)C |
| ASO-1236 | 1257 | moeGpsmoe(5m)CpsmoeApsmoeGpsmoeApsdGpsdGpsdTpsdGpsdApsdXpsd |
| | | Gpsd(5m)CpsdGpsdApsmoeApslnGpslnTpsmoeGpsmoe(5m)C |
| ASO-1237 | 1258 | moeGpsmoe(5m)CpsmoeApsmoeGpsmoeApsdGpsdGpsdTpsdGpsdApsdXpsd |
| | | Gpsd(5m)CpsdGpsdApsmoeApslnGpsmoeTpslnGpsmoe(5m)C |
| ASO-1238 | 1259 | moeGpsmoe(5m)CpsmoeApsmoeGpsmoeApsdGpsdGpsdTpsdGpsdApsdXpsd |
| | | Gpsd(5m)CpsdGpsdApsmoeApslnGpsmoeTpsmoeGpsln(5m)C |
| ASO-1239 | 1260 | moeGpsmoe(5m)CpsmoeApsmoeGpsmoeApsdGpsdGpsdTpsdGpsdApsdXpsd |
| | | Gpsd(5m)CpsdGpsdApsmoeApsmoeGpsmoeTpslnGpsln(5m)C |
| ASO-1240 | 1261 | monGalNAc4-p-moeGpsmoe(5m)CpsmoeApsmoeGpslnApsGpsGpsTpsGps |
| | | ApsmXpsGps(5m)CpsGpsApsmoeApsmoeGpslnTpsmoeGpsmoe(5m)C |
| ASO-1241 | 1262 | monGalNAc4-p-moeGpsmoe(5m)CpsmoeApslnGpsmoeApsGpsGpsTpsGps |
| | | ApsdXpsGps(5m)CpsGpsApslnApsmoeGpsmoeTpsmoeGpsmoe(5m)C |
| ASO-1242 | 1263 | monGalNAc4-p-moeGpsmoe(5m)CpsmoeApsmoeGpsmoeApsGpsGpsTpsGps |
| | | ApsdXpsGps(5m)CpsGpsApslnApsmoeGpslnTpsmoeGpsmoe(5m)C |
| ASO-1243 | 1264 | monGalNAc4-p-moeGpsln(5m)CpsmoeApsmoeGpsmoeApsGpsGpsTpsGps |
| | | ApsmXpsGps(5m)CpsGpsApslnApsmoeGpsmoeTpsmoeGpsmoe(5m)C |
| ASO-1244 | 1265 | monGalNAc4-p-moeGpsmoe(5m)CpsmoeApsmoeGpsmoeApsGpsGpsTpsGps |
| | | ApsdXpsGps(5m)CpsGpsApsmoeApsmoeGpsmoeTpsmoeGpsmoe(5m)C |
| ASO-1245 | 1266 | moeGpsmoe(5m)CpsmoeApsmoeGpslnApsGpsGpsTpsGpsApsdXpsGps(5m) |
| | | CpsGpsApsmoeApsmoeGpsmoeTpsmoeGpsln(5m)C |
| ASO-1246 | 1267 | moeGpsmoe(5m)CpsmoeApsmoeGpsmoeApsGpsGpsTpsGpsApsdXpsGps |
| | | (5m)CpsGpsApslnApslnGpsmoeTpsmoeGpsmoe(5m)C |
| ASO-1247 | 1268 | monGalNAc4-p-moeGpsmoe(5m)CpsmoeApsmoeGpsmoeApsGpsGpsTpsGps |
| | | ApsmXpsGps(5m)CpsGpsApslnApsmoeGpsmoeTpslnGpsmoe(5m)C |
| ASO-1248 | 1269 | monGalNAc4-p-moeGpsmoe(5m)CpslnApsmoeGpslnApsGpsGpsTpsGpsApsd |
| | | XpsGps(5m)CpsGpsApsmoeApsmoeGpsmoeTpsmoeGpsmoe(5m)C |
| ASO-1249 | 1270 | moeGpsmoe(5m)CpsmoeApsmoeGpsmoeApsmoeXpsGpsTpsGpsApsApsGps |
| | | (5m)CpsGpsApsmoeApsmoeGpsmoeTpsmoeGpsmoe(5m)C |
| ASO-1250 | 1271 | moeGpsmoe(5m)CpsmoeApsmoeGpsmoeApsGpsmoeXpsTpsGpsApsApsGps |
| | | (5m)CpsGpsApsmoeApsmoeGpsmoeTpsmoeGpsmoe(5m)C |
| ASO-1251 | 1272 | moeGpsmoe(5m)CpsmoeApsmoeGpsmoeApsGpsGpsmoeXpsGpsApsApsGps |
| | | (5m)CpsGpsApsmoeApsmoeGpsmoeTpsmoeGpsmoe(5m)C |
| ASO-1252 | 1273 | moeGpsmoe(5m)CpsmoeApsmoeGpsmoeApsGpsGpsTpsmoeXpsApsApsGps |
| | | (5m)CpsGpsApsmoeApsmoeGpsmoeTpsmoeGpsmoe(5m)C |
| ASO-1253 | 1274 | moeGpsmoe(5m)CpsmoeApsmoeGpsmoeApsGpsGpsTpsGpsmoeXpsApsGps |
| | | (5m)CpsGpsApsmoeApsmoeGpsmoeTpsmoeGpsmoe(5m)C |
| ASO-1254 | 1275 | moeGpsmoe(5m)CpsmoeApsmoeGpsmoeApsGpsGpsTpsGpsApsmoeXpsGps |
| | | (5m)CpsGpsApsmoeApsmoeGpsmoeTpsmoeGpsmoe(5m)C |
| ASO-1255 | 1276 | moeGpsmoe(5m)CpsmoeApsmoeGpsmoeApsGpsGpsTpsGpsApsApsmoeXps |
| | | (5m)CpsGpsApsmoeApsmoeGpsmoeTpsmoeGpsmoe(5m)C |
| ASO-1256 | 1277 | moeGpsmoe(5m)CpsmoeApsmoeGpsmoeApsGpsGpsTpsGpsApsApsGpsmoe |
| | | XpsGpsApsmoeApsmoeGpsmoeTpsmoeGpsmoe(5m)C |
| ASO-1257 | 1278 | moeGpsmoe(5m)CpsmoeApsmoeGpsmoeApsGpsGpsTpsGpsApsApsGps(5m) |
| | | CpsmoeXpsApsmoeApsmoeGpsmoeTpsmoeGpsmoe(5m)C |
| ASO-1258 | 1279 | moeGpsmoe(5m)CpsmoeApsmoeGpsmoeApsGpsGpsTpsGpsApsApsGps(5m) |
| | | CpsGpsmoeXpsmoeApsmoeGpsmoeTpsmoeGpsmoe(5m)C |
| ASO-1259 | 1280 | moeGpsmoe(5m)CpsmoeApsmoeGpsmoeApsGpsGpsTpsGpsApsApsGpsln |
| | | XpsGpsApsmoeApsmoeGpsmoeTpsmoeGpsmoe(5m)C |
| ASO-1260 | 1281 | moeGpsmoe(5m)CpsmoeApsmoeGpsmoeApsGpsGpsTpsGpsApsApslnXps |
| | | (5m)CpsGpsApsmoeApsmoeGpsmoeTpsmoeGpsmoe(5m)C |
| ASO-1261 | 1282 | moeGpsmoe(5m)CpsmoeApsmoeGpsmoeApsGpsGpsTpsGpsApslnXpsGps |
| | | (5m)CpsGpsApsmoeApsmoeGpsmoeTpsmoeGpsmoe(5m)C |
| ASO-1262 | 1283 | moeGpsmoe(5m)CpsmoeApsmoeGpsmoeApsGpsGpsTpsGpslnXpsApsGps |
| | | (5m)CpsGpsApsmoeApsmoeGpsmoeTpsmoeGpsmoe(5m)C |
| ASO-1263 | 1284 | moeGpsmoe(5m)CpsmoeApsmoeGpsmoeApsGpsGpsTpslnXpsApsApsGps |
| | | (5m)CpsGpsApsmoeApsmoeGpsmoeTpsmoeGpsmoe(5m)C |
| ASO-1264 | 1285 | moeGpsmoe(5m)CpsmoeApsmoeGpsmoeApsGpsGpslnXpsGpsApsApsGps |
| | | (5m)CpsGpsApsmoeApsmoeGpsmoeTpsmoeGpsmoe(5m)C |
| ASO-1265 | 1286 | moeGpsmoe(5m)CpsmoeApsmoeGpsmoeApsGpslnXpsTpsGpsApsApsGps |
| | | (5m)CpsGpsApsmoeApsmoeGpsmoeTpsmoeGpsmoe(5m)C |
| ASO-1266 | 1287 | moeGpsmoe(5m)CpsmoeApsmoeGpsmoeApslnXpsGpsTpsGpsApsApsGps |
| | | (5m)CpsGpsApsmoeApsmoeGpsmoeTpsmoeGpsmoe(5m)C |
| ASO-1267 | 1288 | moeGpsmoe(5m)CpsmoeApsmoeGpsmoeApsGpsGpsTpsGpsApsApsGps(5m) |
| | | CpsGpslnXpsmoeApsmoeGpsmoeTpsmoeGpsmoe(5m)C |
| ASO-1268 | 1289 | moeGpsmoe(5m)CpsmoeApsmoeGpsmoeApsGpsGpsTpsGpsApsApsGps(5m) |
| | | CpslnXpsApsmoeApsmoeGpsmoeTpsmoeGpsmoe(5m)C |
| ASO-1269 | 1290 | moeGpsmoe(5m)CpsmoeApslnGpsmoeApsGpsGpsTpsGpsmXpsApsGps(5m) |
| | | CpsGpsApsmoeApsmoeGpsmoeTpsmoeGpsmoe(5m)C |
| ASO-1270 | 1291 | moeGpsmoe(5m)CpslnApsmoeGpsmoeApsGpsGpsTpsGpsmXpsApsGps(5m) |
| | | CpsGpsApsmoeApsmoeGpsmoeTpsmoeGpsmoe(5m)C |
| ASO-1271 | 1292 | moeGpsln(5m)CpsmoeApsmoeGpsmoeApsGpsGpsTpsGpsmXpsApsGps(5m) |
| | | CpsGpsApsmoeApsmoeGpsmoeTpsmoeGpsmoe(5m)C |
| ASO-1272 | 1293 | lnGpsmoe(5m)CpsmoeApsmoeGpsmoeApsGpsGpsTpsGpsmXpsApsGps(5m) |
| | | CpsGpsApsmoeApsmoeGpsmoeTpsmoeGpsmoe(5m)C |
| ASO-1273 | 1294 | moeGpsmoe(5m)CpslnApsmoeGpsmoeApsGpsGpsTpsGpsmXpsApsGps(5m) |
| | | CpsGpsApsmoeApsmoeGpsmoeTpsmoeGpsln(5m)C |
| ASO-1274 | 1295 | moeGpsmoe(5m)CpslnApsmoeGpsmoeApsGpsGpsTpsGpsmXpsApsGps(5m) |
| | | CpsGpsApsmoeApsmoeGpsmoeTpslnGpsmoe(5m)C |
| ASO-1275 | 1296 | moeGpsmoe(5m)CpslnApsmoeGpsmoeApsGpsGpsTpsGpsmXpsApsGps(5m) |
| | | CpsGpsApsmoeApsmoeGpslnTpsmoeGpsmoe(5m)C |
| ASO-1276 | 1297 | moeGpsmoe(5m)CpslnApsmoeGpsmoeApsGpsGpsTpsGpsmXpsApsGps(5m) |
| | | CpsGpsApsmoeApslnGpsmoeTpsmoeGpsmoe(5m)C |
| ASO-1277 | 1298 | moeGpsmoe(5m)CpslnApsmoeGpsmoeApsGpsGpsTpsGpsmXpsApsGps(5m) |
| | | CpsGpsApslnApsmoeGpsmoeTpsmoeGpsmoe(5m)C |
| ASO-1278 | 1299 | moeGpsmoe(5m)CpslnApsmoeGpslnApsGpsGpsTpsGpsmXpsApsGps(5m)Cps |
| | | GpsApsmoeApsmoeGpsmoeTpsmoeGpsmoe(5m)C |
| ASO-1279 | 1300 | moeGpsmoe(5m)CpslnApslnGpsmoeApsGpsGpsTpsGpsmXpsApsGps(5m)Cps |
| | | GpsApsmoeApsmoeGpsmoeTpsmoeGpsmoe(5m)C |
| ASO-1280 | 1301 | moeGpsln(5m)CpsmoeApsmoeGpsmoeApsGpsGpsTpsGpsmXpsApsGps(5m) |
| | | CpsGpsApsmoeApsmoeGpsmoeTpsmoeGpsln(5m)C |
| ASO-1281 | 1302 | moeGpsln(5m)CpsmoeApsmoeGpsmoeApsGpsGpsTpsGpsmXpsApsGps(5m) |
| | | CpsGpsApsmoeApsmoeGpsmoeTpslnGpsmoe(5m)C |
| ASO-1282 | 1303 | moeGpsln(5m)CpsmoeApsmoeGpsmoeApsGpsGpsTpsGpsmXpsApsGps(5m) |
| | | CpsGpsApsmoeApsmoeGpslnTpsmoeGpsmoe(5m)C |
| ASO-1283 | 1304 | moeGpsln(5m)CpsmoeApsmoeGpsmoeApsGpsGpsTpsGpsmXpsApsGps(5m) |
| | | CpsGpsApsmoeApslnGpsmoeTpsmoeGpsmoe(5m)C |
| ASO-1284 | 1305 | moeGpsln(5m)CpsmoeApsmoeGpsmoeApsGpsGpsTpsGpsmXpsApsGps(5m) |
| | | CpsGpsApslnApsmoeGpsmoeTpsmoeGpsmoe(5m)C |
| ASO-1285 | 1306 | moeGpsln(5m)CpsmoeApsmoeGpslnApsGpsGpsTpsGpsmXpsApsGps(5m)Cps |
| | | GpsApsmoeApsmoeGpsmoeTpsmoeGpsmoe(5m)C |
| ASO-1286 | 1307 | moeGpsln(5m)CpsmoeApslnGpsmoeApsGpsGpsTpsGpsmXpsApsGps(5m)Cps |
| | | GpsApsmoeApsmoeGpsmoeTpsmoeGpsmoe(5m)C |
| ASO-1287 | 1308 | moeGpsln(5m)CpslnApsmoeGpsmoeApsGpsGpsTpsGpsmXpsApsGps(5m)Cps |
| | | GpsApsmoeApsmoeGpsmoeTpsmoeGpsmoe(5m)C |
| ASO-1288 | 1309 | lnGpsmoe(5m)CpsmoeApsmoeGpsmoeApsGpsGpsTpsGpsmXpsApsGps(5m) |
| | | CpsGpsApsmoeApsmoeGpsmoeTpsmoeGpsln(5m)C |
| ASO-1289 | 1310 | lnGpsmoe(5m)CpsmoeApsmoeGpsmoeApsGpsGpsTpsGpsmXpsApsGps(5m) |
| | | CpsGpsApsmoeApsmoeGpsmoeTpslnGpsmoe(5m)C |
| ASO-1290 | 1311 | lnGpsmoe(5m)CpsmoeApsmoeGpsmoeApsGpsGpsTpsGpsmXpsApsGps(5m) |
| | | CpsGpsApsmoeApsmoeGpslnTpsmoeGpsmoe(5m)C |
| ASO-1291 | 1312 | lnGpsmoe(5m)CpsmoeApsmoeGpsmoeApsGpsGpsTpsGpsmXpsApsGps(5m) |
| | | CpsGpsApsmoeApslnGpsmoeTpsmoeGpsmoe(5m)C |
| ASO-1292 | 1313 | lnGpsmoe(5m)CpsmoeApsmoeGpsmoeApsGpsGpsTpsGpsmXpsApsGps(5m) |
| | | CpsGpsApslnApsmoeGpsmoeTpsmoeGpsmoe(5m)C |
| ASO-1293 | 1314 | lnGpsmoe(5m)CpsmoeApsmoeGpslnApsGpsGpsTpsGpsmXpsApsGps(5m)Cps |
| | | GpsApsmoeApsmoeGpsmoeTpsmoeGpsmoe(5m)C |
| ASO-1294 | 1315 | lnGpsmoe(5m)CpsmoeApslnGpsmoeApsGpsGpsTpsGpsmXpsApsGps(5m)Cps |
| | | GpsApsmoeApsmoeGpsmoeTpsmoeGpsmoe(5m)C |
| ASO-1295 | 1316 | lnGpsmoe(5m)CpslnApsmoeGpsmoeApsGpsGpsTpsGpsmXpsApsGps(5m)Cps |
| | | GpsApsmoeApsmoeGpsmoeTpsmoeGpsmoe(5m)C |
| ASO-1296 | 1317 | lnGpsln(5m)CpsmoeApsmoeGpsmoeApsGpsGpsTpsGpsmXpsApsGps(5m)Cps |
| | | GpsApsmoeApsmoeGpsmoeTpsmoeGpsmoe(5m)C |
| ASO-1297 | 1318 | moeGpsmoe(5m)CpsmoeApsmoeGpsmoeApsGpsGpsTpsGpsmXpsApsGps |
| | | (5m)CpsGpsApsmoeApsmoeGpsmoeTpsmoeGpsln(5m)C |
| ASO-1298 | 1319 | moeGpsmoe(5m)CpsmoeApsmoeGpsmoeApsGpsGpsTpsGpsmXpsApsGps |
| | | (5m)CpsGpsApsmoeApsmoeGpsmoeTpslnGpsmoe(5m)C |
| ASO-1299 | 1320 | moeGpsmoe(5m)CpsmoeApsmoeGpsmoeApsGpsGpsTpsGpsmXpsApsGps |
| | | (5m)CpsGpsApsmoeApsmoeGpslnTpsmoeGpsmoe(5m)C |
| ASO-1300 | 1321 | moeGpsmoe(5m)CpsmoeApsmoeGpsmoeApsGpsGpsTpsGpsmXpsApsGps |
| | | (5m)CpsGpsApsmoeApslnGpsmoeTpsmoeGpsmoe(5m)C |
| ASO-1301 | 1322 | moeGpsmoe(5m)CpsmoeApsmoeGpsmoeApsGpsGpsTpsGpsmXpsApsGps |
| | | (5m)CpsGpsApslnApsmoeGpsmoeTpsmoeGpsmoe(5m)C |
| ASO-1302 | 1323 | moeGpsmoe(5m)CpsmoeApsmoeGpslnApsGpsGpsTpsGpsmXpsApsGps(5m) |
| | | CpsGpsApsmoeApsmoeGpsmoeTpsmoeGpsmoe(5m)C |
| ASO-1303 | 1324 | moeGpsmoe(5m)CpsmoeApslnGpslnApsGpsGpsTpsGpsmXpsApsGps(5m)Cps |
| | | GpsApsmoeApsmoeGpsmoeTpsmoeGpsmoe(5m)C |
| ASO-1304 | 1325 | moeGpsmoe(5m)CpsmoeApslnGpsmoeApsGpsGpsTpsGpsmXpsApsGps(5m) |
| | | CpsGpsApslnApsmoeGpsmoeTpsmoeGpsmoe(5m)C |
| ASO-1305 | 1326 | moeGpsmoe(5m)CpsmoeApslnGpsmoeApsGpsGpsTpsGpsmXpsApsGps(5m) |
| | | CpsGpsApsmoeApslnGpsmoeTpsmoeGpsmoe(5m)C |
| ASO-1306 | 1327 | moeGpsmoe(5m)CpsmoeApslnGpsmoeApsGpsGpsTpsGpsmXpsApsGps(5m) |
| | | CpsGpsApsmoeApsmoeGpslnTpsmoeGpsmoe(5m)C |
| ASO-1307 | 1328 | moeGpsmoe(5m)CpsmoeApslnGpsmoeApsGpsGpsTpsGpsmXpsApsGps(5m) |
| | | CpsGpsApsmoeApsmoeGpsmoeTpslnGpsmoe(5m)C |
| ASO-1308 | 1329 | moeGpsmoe(5m)CpsmoeApslnGpsmoeApsGpsGpsTpsGpsmXpsApsGps(5m) |
| | | CpsGpsApsmoeApsmoeGpsmoeTpsmoeGpsln(5m)C |
| ASO-1309 | 1330 | moeGpsmoe(5m)CpsmoeApsmoeGpslnApsGpsGpsTpsGpsmXpsApsGps(5m) |
| | | CpsGpsApslnApsmoeGpsmoeTpsmoeGpsmoe(5m)C |
| ASO-1310 | 1331 | moeGpsmoe(5m)CpsmoeApsmoeGpslnApsGpsGpsTpsGpsmXpsApsGps(5m) |
| | | CpsGpsApsmoeApslnGpsmoeTpsmoeGpsmoe(5m)C |
| ASO-1311 | 1332 | moeGpsmoe(5m)CpsmoeApsmoeGpslnApsGpsGpsTpsGpsmXpsApsGps(5m) |
| | | CpsGpsApsmoeApsmoeGpslnTpsmoeGpsmoe(5m)C |
| ASO-1312 | 1333 | moeGpsmoe(5m)CpsmoeApsmoeGpslnApsGpsGpsTpsGpsmXpsApsGps(5m) |
| | | CpsGpsApsmoeApsmoeGpsmoeTpslnGpsmoe(5m)C |
| ASO-1313 | 1334 | moeGpsmoe(5m)CpsmoeApsmoeGpslnApsGpsGpsTpsGpsmXpsApsGps(5m) |
| | | CpsGpsApsmoeApsmoeGpsmoeTpsmoeGpsln(5m)C |
| ASO-1314 | 1335 | moeGpsmoe(5m)CpsmoeApsmoeGpsmoeApsGpsGpsTpsGpsmXpsApsGps |
| | | (5m)CpsGpsApslnApslnGpsmoeTpsmoeGpsmoe(5m)C |
| ASO-1315 | 1336 | moeGpsmoe(5m)CpsmoeApsmoeGpsmoeApsGpsGpsTpsGpsmXpsApsGps |
| | | (5m)CpsGpsApslnApsmoeGpslnTpsmoeGpsmoe(5m)C |
| ASO-1316 | 1337 | moeGpsmoe(5m)CpsmoeApsmoeGpsmoeApsGpsGpsTpsGpsmXpsApsGps |
| | | (5m)CpsGpsApslnApsmoeGpsmoeTpslnGpsmoe(5m)C |
| ASO-1317 | 1338 | moeGpsmoe(5m)CpsmoeApsmoeGpsmoeApsGpsGpsTpsGpsmXpsApsGps |
| | | (5m)CpsGpsApslnApsmoeGpsmoeTpsmoeGpsln(5m)C |
| ASO-1318 | 1339 | moeGpsmoe(5m)CpsmoeApsmoeGpsmoeApsGpsGpsTpsGpsmXpsApsGps |
| | | (5m)CpsGpsApsmoeApslnGpslnTpsmoeGpsmoe(5m)C |
| ASO-1319 | 1340 | moeGpsmoe(5m)CpsmoeApsmoeGpsmoeApsGpsGpsTpsGpsmXpsApsGps |
| | | (5m)CpsGpsApsmoeApslnGpsmoeTpslnGpsmoe(5m)C |
| ASO-1320 | 1341 | moeGpsmoe(5m)CpsmoeApsmoeGpsmoeApsGpsGpsTpsGpsmXpsApsGps |
| | | (5m)CpsGpsApsmoeApslnGpsmoeTpsmoeGpsln(5m)C |
| ASO-1321 | 1342 | moeGpsmoe(5m)CpsmoeApsmoeGpsmoeApsGpsGpsTpsGpsmXpsApsGps |
| | | (5m)CpsGpsApsmoeApsmoeGpslnTpslnGpsmoe(5m)C |
| ASO-1322 | 1343 | moeGpsmoe(5m)CpsmoeApsmoeGpsmoeApsGpsGpsTpsGpsmXpsApsGps |
| | | (5m)CpsGpsApsmoeApsmoeGpslnTpsmoeGpsln(5m)C |
| ASO-1323 | 1344 | moeGpsmoe(5m)CpsmoeApsmoeGpsmoeApsGpsGpsTpsGpsmXpsApsGps |
| | | (5m)CpsGpsApsmoeApsmoeGpsmoeTpslnGpsln(5m)C |

Nucleotides included in Table 1 should be considered deoxynucleotides unless indicated to the contrary. Thus a base identifier (i.e., A, T, G, C, or U) refers to a deoxynucleotide if not modified by another code letter (e.g., r, ocp, lm, etc.). Alternatively, some sequences may also include a ″d″ in addition to the base identifier (e.g., dG, dT, dA, dC, etc.), which is also indicative of a deoxynucleotide. 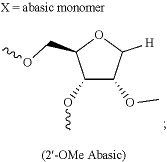 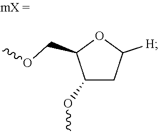
dX = (2′-deoxy Abasic); 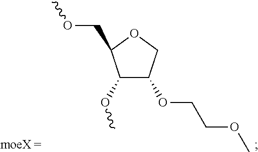 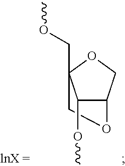
ps = phosphorothioate linkage;
yp = mesyl phosphoroamidate linkage;
pss? = S-stereo-defined phosphorothioate linkage;
psr? = R-stereo-defined phosphorothioate linkage;
moe = 2′-O-methoxyethyl;
(5m)C = 5 methylcytosine;
mA, mG, mT, mU, m(5m)C and mC = 2′-O-methyl nucleotide;
rA, rG, rC = ribonucleotide;
ocp = 2′-O-cyclopropyl;
lm = 2′-OMe-3′-xylo;
omcp = 2′-O-methylcyclopropyl;
DAP = 2,6-diaminopurine;
(2s)T = 2-thio-thymine;
(8nh)G = 8-amio-guanosine;
(8nh)A = 8-amino-adenosine;
(5oh)C = 5′-hydroxy-cytosine;
ln = LNA;
gutb = tert butyl GuNA;

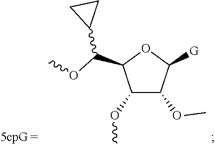

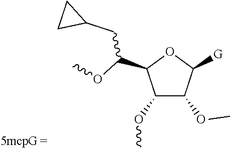

TABLE 2

| SEQ ID NO: | Unmodified ASO Sequences |
|---|---|
| 321 | GCAGAGGTGAAGCGAAGTGC |
| 322 | GTGAAGCGAAGTGCACACGG |
| 323 | GGTGAAGCGAAGTGCACACG |
| 324 | AGGTGAAGCGAAGTGCACAC |
| 325 | GAGGTGAAGCGAAGTGCACA |
| 326 | AGAGGTGAAGCGAAGTGCAC |
| 327 | CAGAGGTGAAGCGAAGTGCA |
| 328 | TGCAGAGGTGAAGCGAAGTG |
| 329 | GTGCAGAGGTGAAGCGAAGT |
| 330 | CGTGCAGAGGTGAAGCGAAG |
| 331 | ACGTGCAGAGGTGAAGCGAA |
| 332 | TTGGCAGAGGTGAAGCGAAGTGC |
| 333 | TGTGCAGAGGTGAAGCGAAGTGC |
| 334 | TGCAGAGGTGAAGCGAAGTGC |
| 335 | TTGCAGAGGTGAAGCGAAGTGC |
| 336 | TTTGCAGAGGTGAAGCGAAGTGC |
| 337 | TGGCAGAGGTGAAGCGAAGTGC |
| 338 | TTGTGCAGAGGTGAAGCGAAGTGC |
| 339 | TGTTGTGCAGAGGTGAAGCGAAGTGC |
| 340 | TTATGCAGAGGTGAAGCGAAGTGC |
| 341 | TATTATGCAGAGGTGAAGCGAAGTGC |
| 342 | GCAGAGGTGAAGCGAAGTGCT |
| 343 | GCAGAGGTGAAGCGAAGTGCTT |
| 344 | GCAGAGGTGAAGCGAAGTGCTTT |
| 345 | GCAGAGGTGAAGCGAAGTGCTG |
| 346 | GCAGAGGTGAAGCGAAGTGCTTG |
| 347 | GCAGAGGTGAAGCGAAGTGCTGT |
| 348 | GCDAPGAGGTGAAGCGAAGTGC |
| 349 | GCAGDAPGGTGAAGCGAAGTGC |
| 350 | GCAGAGGTGAAGCGADAPGTGC |
| 351 | CAGAGGTGAAGCGAAGTGC |
| 352 | GCAGAGGTGAAGCGAAGTG |

### III. Antisense Oligonucleotide Functions

Provided herein are antisense nucleotides that are capable of mediating degradation of RNA transcripts and activating the immune pathways of the subject, while not resulting in elevated levels of cell death.

Without wishing to be bound by any particular theory, it is believed that ASOs hybridize to target RNA and then mediate degradation of target RNA transcripts via recruitment of RNase H to the DNA/RNA heteroduplex. ASOs may also trigger activation of PRRs and pathways, including TLR8. ASOs may display off-target effects by binding to transcripts other than the target RNA transcript, which may result in cellular toxicity. Binding to proteins within the cells, which may disrupt normal cell function and result in cytotoxicity. Activation of proinflammatory mechanisms by ASOs could also contribute to side effects. Main ASO target organs such as the liver and kidneys, are where the ASO related toxic effects could be more pronounced. Thus, an important consideration when developing ASOs for therapeutic purposes is to balance the performance of the ASO in all these areas to select candidates based on their ability to cause RNA transcript degradation and moderate immune activation while avoiding increased cytotoxicity.

### RNase H Activity

As provided in the Examples included herein, the disclosed ASO can mediate degradation of target RNA via an RNase H-mediated pathway.

Antisense oligonucleotides can hybridize to target RNA transcripts to form an ASO/RNA complex. RNase H is recruited to these complexes and cleaves the RNA, resulting in degradation of the transcripts. This RNA transcript degradation results in a reduction of HBV-derived RNAs and viral proteins, which leads to reduced HBV replication and antigen production and could potentially be one key component of combination therapy targeting functional cure for CHB.

RNase H recruitment is known to be affected by the structure of the ASO, including its sequence and modifications. Thus, modifications of an ASO that may increase its stability can improve in vivo potency.

### TLR8 and Additional TLR Activity

As provided in the Examples included herein, the disclosed ASO can activate TLR8.

Toll-like receptor 8 (TLR8) is a receptor that plays a role in the response to chronic viral infections, including HBV infection. TLR8 recognizes single-stranded RNA of viruses or bacteria and triggers production of proinflammatory cytokines, including tumor necrosis factor α (TNF-α) and interleukin-12 (IL-12). Production of these cytokines leads to activation of “exhausted” CD8+ cytolytic T cells, which leads to clearance of the infection. Certain ASOs with unique sequences and chemical modifications were found to activate human TLR8 receptor or potentiate the activation of human TLR8 by small molecule agonist such as R848.

Recently, studies using ASOs to treat HBV discovered that the ASOs were able to trigger TLR8 activation, which presents an additional pathway to control and reduce HBV infection. As this link between ASOs and TLR8-activation has not been well studied, it is unknown how different sequences and modifications of the ASOs, including nucleoside modifications and phosphorothioate linkages, may impact the ability of the ASOs to activate this immune pathway, which improves treatment outcomes for HBV.

### Caspase Activity

As provided in the Examples included herein, the disclosed ASO may possess low amounts of caspase induction activity. Caspases are the major mediators of apoptosis; cells that have high levels of caspase expression or activation are undergoing cell death. Thus, ASOs that trigger less caspase expression or activation trigger less cell death.

### IV. Pharmaceutical Compositions

The present disclosure also encompasses pharmaceutical compositions comprising ASOs of the present disclosure. One embodiment is a pharmaceutical composition comprising one or more ASOs of the present disclosure, and a pharmaceutically acceptable diluent or carrier.

In some embodiments, the pharmaceutical compositions comprise any of the ASOs and nucleotide sequences described herein. The compositions may comprise 1, 2, 3, 4, 5, 6, 7, 8, 9, 10, 11, 12, or more ASOs described herein. The compositions may comprise a nucleotide sequence comprising a nucleotide sequence of any one of SEQ ID NOs: 3-320, 353-404, and 446-1344. In some embodiments, the pharmaceutical composition comprises any one of ASO-676 (SEQ ID NO: 697), ASO-677 (SEQ ID NO: 698), ASO-1037 (SEQ ID NO: 1058), ASO-707 (SEQ ID NO: 728), ASO-1192 (SEQ ID NO: 1213), ASO-651 (SEQ ID NO: 672), ASO-1166 (SEQ ID NO: 1187), ASO-1181 (SEQ ID NO: 1202), ASO-1179 (SEQ ID NO: 1200), and ASO-962 (SEQ ID NO: 983). In some embodiments, the ASO is selected from ASO-676 (SEQ ID NO: 697), ASO-677 (SEQ ID NO: 698), ASO-1037 (SEQ ID NO: 1058), ASO-707 (SEQ ID NO: 728), and ASO-1192 (SEQ ID NO: 1213). In some embodiments, the ASO is selected from ASO-651 (SEQ ID NO: 672), ASO-1166 (SEQ ID NO: 1187), ASO-1181 (SEQ ID NO: 1202), and ASO-1179 (SEQ ID NO: 1200). In some embodiments, the ASO is ASO-962 (SEQ ID NO: 983).

In some embodiments, the pharmaceutical composition containing the ASO of the present disclosure is formulated for systemic administration via parenteral delivery. Parenteral administration includes intravenous, intra-arterial, subcutaneous, intraperitoneal or intramuscular injection or infusion; also subdermal administration, e.g., via an implanted device. In a preferred embodiment, the pharmaceutical composition containing the ASO of the present disclosure is formulated for subcutaneous (SC) or intravenous (IV) delivery. Formulations for parenteral administration may include sterile aqueous solutions, which may also contain buffers, diluents and other pharmaceutically acceptable additives as understood by the skilled artisan. For intravenous use, the total concentration of solutes may be controlled to render the preparation isotonic.

The pharmaceutical composition containing the ASO of the present disclosure is useful for treating a disease or disorder, e.g., associated with the expression or activity of a hepatitis B virus gene, such as X gene or S gene.

In some embodiments, the pharmaceutical composition comprises an ASO of the present disclosure that is complementary or hybridizes to a viral target RNA sequence (e.g., HBV target gene), and a pharmaceutically acceptable diluent or carrier. When the pharmaceutical composition comprises two or more ASOs, the ASOs may be present in varying amounts. For example, in some embodiments, the weight ratio of first ASO to second ASO is 1:4 to 4:1, e.g., 1:4, 1:3, 1:2, 1:1, 2:1, 3:1, or 4:1. In some embodiments, the molar ratio of first ASO to second ASO is 1:4 to 4:1, e.g., 1:4, 1:3, 1:2, 1:1, 2:1, 3:1, or 4:1.

In some embodiments, the pharmaceutical composition comprises an amount of one or more of the ASOs described herein formulated with one or more pharmaceutically acceptable carriers (additives) and/or diluents. The pharmaceutical compositions may be specially formulated for administration in solid or liquid form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) intravaginally or intrarectally, for example, as a pessary, cream or foam; (4) sublingually; (5) ocularly; (6) transdermally; or (7) nasally.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present disclosure include those suitable for nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound (e.g., ASO) which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In some embodiments, a formulation of the present disclosure comprises an excipient selected from the group consisting of cyclodextrins, celluloses, liposomes, micelle forming agents, e.g., bile acids, and polymeric carriers, e.g., polyesters and polyanhydrides; and a compound (e.g., ASO) of the present disclosure.

Methods of preparing these formulations or compositions include the step of bringing into association a compound (e.g., ASO) of the present disclosure with the carrier and, optionally, one or more accessory ingredients. In general, the formulations are prepared by uniformly and intimately bringing into association a compound (e.g., ASO) of the present disclosure with liquid carriers, or finely divided solid carriers, or both, and then, if necessary, shaping the product.

Formulations of the disclosure suitable for a suspension in an aqueous or non-aqueous liquid, or as an oil-in-water or water-in-oil liquid emulsion, or as an elixir or syrup, each containing a predetermined amount of a compound (e.g., ASO) of the present disclosure as an active ingredient. A compound (e.g., ASO) of the present disclosure may also be administered as a bolus, electuary, or paste.

In dosage forms of the disclosure, the active ingredient may be mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose.

The disclosed dosage forms may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms of the compounds (e.g., ASO) of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (I particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the compositions can also include adjuvants such as wetting agents, emulsifying and suspending agents, sweetening, flavoring, coloring, perfuming and preservative agents.

Suspensions, in addition to the active compounds (e.g., ASO), may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the disclosure for rectal or vaginal administration may be presented as a suppository, which may be prepared by mixing one or more compounds (e.g., ASO) of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound (e.g., ASO).

Formulations of the present disclosure which are suitable for vaginal administration also include pessaries, tampons, creams, gels, pastes, foams or spray formulations containing such carriers as are known in the art to be appropriate.

Dosage forms for the topical or transdermal administration of a compound (e.g., ASO) of this disclosure include powders, sprays, ointments, pastes, creams, lotions, gels, solutions, patches and inhalants. The active compound (e.g., ASO) may be mixed under sterile conditions with a pharmaceutically acceptable carrier, and with any preservatives, buffers, or propellants which may be required.

The ointments, pastes, creams and gels may contain, in addition to an active compound (e.g., ASO) of this disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound (e.g., ASO) of this disclosure, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound (e.g., ASO) of the present disclosure to the body. Such dosage forms can be made by dissolving or dispersing the compound (e.g., ASO) in the proper medium. Absorption enhancers can also be used to increase the flux of the compound (e.g., ASO) across the skin. The rate of such flux can be controlled by either providing a rate controlling membrane or dispersing the compound (e.g., ASO) in a polymer matrix or gel.

Ophthalmic formulations, eye ointments, powders, solutions and the like, are also contemplated as being within the scope of this disclosure.

Pharmaceutical compositions of this disclosure suitable for parenteral administration comprise one or more compounds (e.g., ASO) of the disclosure in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds (e.g., ASO) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds (e.g., ASO) of the present disclosure are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

### V. Treatments

Disclosed herein are methods of treatment or prevention of a disease or disorder in a subject in need thereof using the ASOs described above. Further disclosed herein are methods of treating an infection (e.g., HBV infection) in a subject in need thereof, the method comprising administering to the subject any of the ASOs described herein. Further disclosed herein are uses of any of the ASOs described herein in the manufacture of a medicament for treating an infection (e.g., HBV infection).

In some embodiments, a method of treating or preventing a disease in a subject in need thereof comprises administering to the subject any of the ASOs disclosed herein. In some embodiments, a method of treating or preventing a disease in a subject in need thereof comprises administering to the subject any of the compositions disclosed herein.

In some embodiments, the subject is a mammal. In some embodiments, the subject is a human. In some embodiments, the subject is a non-human primate. In some embodiments, the subject is a cat. In some embodiments, the subject is a camel. In preferred embodiments in which the subject is a human, the subject may be at least 40 years old, at least 45 years old, at least 50 years old, at least 55 years old, at least 60 years old, at least 65 years old, at least 70 years old, at least 75 years old, or at least 80 years old or older. In some embodiments, the subject is a pediatric subject (i.e., less than 18 years old).

The preparations (e.g., ASOs or pharmaceutical compositions thereof) of the present disclosure may be given parenterally, topically, or rectally or administered in the form of an inhalant. They are, of course, given in forms suitable for each administration route. For example, they are administered in tablets or capsule form, administration by injection, infusion, or inhalation; topical by lotion or ointment; rectal by suppositories. Injection, infusion, or inhalation are preferred.

These compounds may be administered to humans and other animals for therapy or as a prophylactic by any suitable route of administration, including nasally (as by, for example, a spray), rectally, intravaginally, parenterally, intracisternally and topically, as by powders, ointments or drops, including buccally and sublingually. In some embodiments, the compounds or compositions are inhaled, as by, for example, an inhaler, a nebulizer, or in an aerosolized form.

Regardless of the route of administration selected, the compounds (e.g., ASOs) of the present disclosure, which may be used in a suitable hydrated form, and/or the pharmaceutical compositions of the present disclosure, are formulated into pharmaceutically-acceptable dosage forms by conventional methods known to those of skill in the art.

In some embodiments, the present disclosure provides method of treating a hepatitis B virus (HBV) infection, comprising administering to a subject in need thereof a therapeutically effective amount of one or more of the ASOs or a pharmaceutical composition as disclosed herein. In some embodiments, the subject has been treated with one or more additional HBV treatment agents. In some embodiments, the subject is concurrently treated with one or more additional HBV treatment agents.

Actual dosage levels of the active ingredients (e.g., ASO) in the pharmaceutical compositions of this disclosure may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound (e.g., ASO) of the present disclosure employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds (e.g., ASO) of the disclosure employed in the pharmaceutical composition at levels lower than that required in order to achieve the desired therapeutic effect and gradually increase the dosage until the desired effect is achieved.

In general, a suitable daily dose of a compound (e.g., ASO) of the disclosure is the amount of the compound that is the lowest dose effective to produce a therapeutic effect. Such an effective dose generally depends upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. In some embodiments, the compound is administered at a dose equal to or greater than 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, 0.50, 0.55, 0.60, 0.65, 0.7, 0.75, 0.8, 0.85, 0.9, 0.95, or 1 mg/kg. In some embodiments, the compound is administered at a dose equal to or less than 200, 190, 180, 170, 160, 150, 140, 130, 120, 110, 100, 95, 90, 85, 80, 75, 70, 65, 60, 55, 50, 45, 40, 35, 30, 25, 20, or 15 mg/kg. In some embodiments, the total daily dose of the compound is equal to or greater than 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, or 400 mg.

If desired, the effective daily dose of the active compound (e.g., ASO) may be administered as two, three, four, five, six, seven, eight, nine, ten or more doses or sub-doses administered separately at appropriate intervals throughout the day, optionally, in unit dosage forms. In some embodiments, the compound is administered at least 1, 2, 3, 4, 5, 6, 7, 8, 9, 10, 11, 12, 13, 14, or 15 times. Preferred dosing is one administration per day. In some embodiments, the compound is administered at least 1, 2, 3, 4, 5, 6, 7, 8, 9, 10, 11, 12, 13, 14, 15, 16, 17, 18, 19, 20, or 21 times a week. In some embodiments, the compound is administered at least 1, 2, 3, 4, 5, 6, 7, 8, 9, 10, 11, 12, 13, 14, 15, 16, 17, 18, 19, 20, or 21 times a month. In some embodiments, the compound is administered once every 1, 2, 3, 4, 5, 6, 7, 8, 9, 10, 11, 12, 13, 14, 15, 16, 17, 18, 19, 20, or 21 days. In some embodiments, the compound is administered every 3 days. In some embodiments, the compound is administered once every 1, 2, 3, 4, 5, 6, 7, 8, 9, 10, 11, 12, 13, 14, or 15 weeks. In some embodiments, the compound is administered every month. In some embodiments, the compound is administered once every 1, 2, 3, 4, 5, 6, 7, 8, 9, 10, 11, 12, 13, 14, or 15 months. In some embodiments, the compound is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days. In some embodiments, the compound is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 weeks. In some embodiments, the compound is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 months. In some embodiments, the compound is administered at least once a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least once a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the compound is administered at least twice a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least twice a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the compound is administered at least once every two weeks for a period of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least once every two weeks for a period of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the compound is administered at least once every four weeks for a period of at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least once every four weeks for a period of at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months.

In some embodiments, any one of the ASOs or compositions disclosed herein is administered in a particle or viral vector. In some embodiments, the viral vector is a vector of adenovirus, adeno-associated virus (AAV), alphavirus, flavivirus, herpes simplex virus, lentivirus, measles virus, picornavirus, poxvirus, retrovirus, or rhabdovirus. In some embodiments, the viral vector is a recombinant viral vector. In some embodiments, the viral vector is selected from AAVrh.74, AAVrh. 10, AAVrh.20, AAV-1, AAV-2, AAV-3, AAV-4, AAV-5, AAV-6, AAV-7, AAV-8, AAV-9, AAV-10, AAV-11, AAV-12 and AAV-13.

The subject of the described methods may be a mammal, and it includes humans and non-human mammals. In some embodiments, the subject is a human, such as an adult human.

The disclosed ASO can be administered alone or in combination with one or more additional HBV treatment agents and/or antiviral agents. The additional HBV treatment agents and/or antiviral agents may be a small molecule (e.g., a nucleoside analog or a protease inhibitor) or a biologic (e.g., an antibody or peptide). Examples of suitable HBV treatment agents include, but are not limited to, a nucleotide analog, nucleoside analog, a capsid assembly modulator (CAM), a recombinant interferon, an entry inhibitor, a small molecule immunomodulator, and an oligonucleotide therapy. In some embodiments, the additional HBV treatment agent is selected from HBV STOPS™, HBV CAM ALG-000184, ALG-125755, recombinant interferon alpha 2b, IFN-α, PEG-IFN-α-2a, PEG-INF-2b, Pegbing (Mipeginterferon alfa-2b), lamivudine, telbivudine, adefovir dipivoxil, clevudine, entecavir, tenofovir alafenamide, tenofovir disoproxil, JNJ-3989 (ARO-HBV, or GSK5637608), GSK3228836, REP-2139, REP-2165, VIR-2218 (BRII-835, or Elebsiran), AB-729 (Imdurisan), DCR-HBVS (RG6346 or Xalnesiran), BW-20507 (Argo HBV siRNA), HT-101 (Hepa Thera HBV siRNA), OLX703A (Olix HBV siRNA), HRS-5635 (Hengrui HBV siRNA), RBD1016 (Ribo HBV siRNA), TQA3038 (ChiaTai Tianqing HBV siRNA), GLS4, NZ-4, RG7907, EDP-514, ABI-H03733, ABI-H2158, ZM-H1505R, ABI-4334 (CAMs), and ABI-6250 (HDV entry inhibitor). In some embodiments, the oligonucleotide therapy is selected from Nucleic Acid Polymers or S-Antigen Transport-inhibiting Oligonucleotide Polymers (NAPs or STOPS), siRNA, and ASO. In some embodiments, any of the ASOs disclosed herein are co-administered with STOPS. Exemplary STOPS are described in International Publication No. WO2020/097342 and U.S. Publication No. 2020/0147124, both of which are incorporated by reference in their entirety. In some embodiments, the STOPS is ALG-010133. In some embodiments, any of the ASOs disclosed herein are co-administered with tenofovir. In some embodiments, any of the ASOs disclosed herein are co-administered with a CAM. Exemplary CAMs are described in Berke et al., Antimicrob Agents Chemother, 2017, 61 (8): e00560-17, Klumpp, et al., Gastroenterology, 2018, 154 (3): 652-662.e8, International Application Nos. PCT/US2020/017974, PCT/US2020/026116, and PCT/US2020/028349 and U.S. application Ser. Nos. 16/789,298, 16/837,515, and 16/849,851, each which is incorporated by reference in its entirety. In some embodiments, the CAM is ALG-000184, ALG-001075, ALG-001024, JNJ-632, BAY41-4109, ABI-4334, or NVR3-778. In some embodiments, the ASOs and the HBV treatment agent are administered simultaneously. In some embodiments, the ASOs and the HBV treatment agent are administered concurrently. In some embodiments, the ASOs and the HBV treatment agent are administered sequentially. In some embodiments, the ASOs are administered prior to administering the HBV treatment agent. In some embodiments, the ASOs are administered after administering the HBV treatment agent. In some embodiments, the ASOs and the HBV treatment agent are in separate containers. In some embodiments, the ASOs and the HBV treatment agent are in the same container.

When the compounds (e.g., ASOs) described herein are co-administered with another, the effective amount may be less when the compound is used alone.

### EXAMPLES

The present technology is further illustrated by the following Examples, which should not be construed as limiting in any way.

### Example 1: ASO Synthesis

Gapmer ASO Sequences: The DNA, 2′-O-Me, and LNA phosphoramidite monomers were procured from commercially available sources (Hongene Biotech USA Inc.). All the monomers were dried in vacuum desiccator with desiccants (P₂O₅, RT 24h). Universal solid supports (CPG) attached were obtained from ChemGenes corporation. The chemicals and solvents for synthesis workflow were purchased from VWR/Sigma commercially available sources and used without any purification or treatment. Solvent (acetonitrile) and solutions (amidite and activator) were stored over molecular sieves during synthesis.

The control and target oligonucleotide sequences were synthesized on an Expedite 8909 synthesizer using the standard cycle written by the manufacturer with modifications to a few wait steps and modified coupling steps. The solid support was controlled pore glass and the monomers contained standard protecting groups. Each chimeric oligonucleotide was individually synthesized using commercially available 5′-O-(4,4′-dimethoxytrityl)-3′-O-(2-cyanoethyl-N,N-diisopropyl) DNA, 2′-OMe, and or LNA phosphoramidite monomers of 6-N-benzoyladenosine (A^{Bz}), 4-N-acetylcytidine (C^{Bz}), 2-N-isobutyrylguanosine (G^{iBu}), and Uridine (U) or Thymidine (T), according to standard solid phase Phosphoramidite synthesis protocols. The 2′-O-Me-2,6-diaminopurine phosphoramidite was purchased from Glen Research. The phosphoramidites were prepared as 0.1 M solutions in anhydrous acetonitrile. 5-Ethylthiotetrazole was used as activator, 3% dichloroacetic acid in dichloromethane was used to detritylate, acetic anhydride in THF and 16% N-methylimidazole in THF were used to cap, and DDTT ((dimethylamino-methylidene) amino)-3H-1,2,4-dithiazaoline-3-thione was used as the sulfur-transfer agent for the synthesis of oligoribonucleotide phosphorothioates. An extended coupling of 0.1M solution of phosphoramidite in CH₃CN in the presence of 5-(ethylthio)-1H-tetrazole activator to a solid bound oligonucleotide followed by extended capping, oxidation and deprotection afforded modified oligonucleotides. The stepwise coupling efficiency of all modified phosphoramidites was more than 98.5%.

Deprotection and cleavage from the solid support was achieved with mixture of ammonia methylamine (1:1, AMA) for 15 min at 65° C., when the universal linker was used, the deprotection was left for 90 min at 65° C. or solid supports were heated with aqueous ammonia (28%) solution at 55° C. for 8 h to deprotect the base labile protecting groups.

After filtering to remove the solid support, the deprotection solution was removed under vacuum in a GeneVac centrifugal evaporator.

### Example 2: Generation of Initial Parental ASOs

In order to identify additional antisense oligonucleotides (ASOs), a known control ASO that reduces expression of hepatitis B virus (HBV) genes was selected as a starting point for the development of parental ASOs. The parental ASOs, ASO-2 through ASO-9 and ASO-318 and ASO-319, were generated by targeting the HBV X Protein transcript region. Table 1 discloses the modified sequences of the control ASO (ASO-1) and the ten parental ASOs, and Table 2 shows the unmodified sequences.

To test the efficacy of the ASO to activate RNase H-mediated degradation of the target HBV RNA, the ten parental ASOs were compared to the control ASO in the assay disclosed above to measure EC₅₀ values. The EC₅₀ assay measures the concentration of ASO necessary to reduce the transcript levels 50% in relation to an untreated cell control; thus, a reduction in the EC₅₀ value indicates an increased activation capacity for the ASO. As the EC₅₀ assay is not very sensitive, any changes less than two- to three-fold different from the control ASO is considered similar to the RNAse H activation seen utilizing the control ASO-1. To test the potential of the ASOs to trigger cell death, the ten parental ASOs were compared to the control ASO in the assay disclosed above to measure CC₅₀ values. The CC₅₀ assay measures the concentration of the ASO necessary to reduce the cell population 50%; thus, an increased CC₅₀ value represents a decreased cytotoxicity of the ASO.

To test the efficacy of the disclosed ASOs to activate RNase H-mediated degradation activity, HepG2.2.15 cells with integrated HBV genome were maintained in DMEM/F-12 medium with 10% fetal bovine serum (FBS) and 1% penicillin/streptomycin, 1% glutamine, 1% non-essential amino acids, and 1% sodium pyruvate. Cells were maintained at 37° C. in a 5% CO₂ atmosphere. On the day of testing, cells were seeded at a concentration of 45,000 cells per well in collagen-I coated 96-well plates. After four hours of incubation, ASOs were transfected using Lipofectamine® RNAiMAX (Thermo Fisher, Cat #: 13778-150) following the manufacturer's instructions. On day 5 after treatment, supernatants were harvested for secreted HBsAg ELISA (Autobio, Cat #CL0310) measurement, and remaining adhered cells were assayed for viability with CellTiter-Glo (Promega, Cat #G7570).

As shown in Table 3, the RNase H activity of the HBV targeting ASO was expressed as EC₅₀. While several parental ASOs, including ASO-2, ASO-3, ASO-318, ASO-5, ASO-6, ASO-8 and ASO-9, exhibited a reduction in EC₅₀ value, and thus are potentially able to activate RNAse H-mediated degradation, the decrease is not necessarily a dramatic difference from the control ASO (Table 3, Column 2). As shown in Table 3, the cytotoxicity of the HBV targeting ASOs was expressed by CC₅₀. In this case, all parental ASOs displayed equal or greater CC₅₀ values, indicating that the ASOs trigger similar or reduced levels of cytotoxicity (Table 3, Column 3).

It was shown that the control ASO-1 was able to activate the TLR8 pathway, which is associated with the control and reduction of HBV infection in cells. In order to determine if any parental ASOs have similar or increased activation of TLR8, the control ASO and parental ASOs were subjected to the TLR8 assay described below. The values for the parental ASOs were normalized to the level of TLR8 activation of the control ASO. Parental ASOs ASO-2, ASO-3, ASO-4, ASO-318, ASO-5, ASO-7, and ASO-319 exhibited decreased activation of TLR8, while ASO-6, ASO-8, and ASO-9 exhibited increased activation of the same pathway (Table 3, Column 4).

To test the efficacy of the disclosed ASOs to activate the TLR8 pathway, HEK Blue hTLR8 (Invivogen; hkb-htlr8) were maintained in Dulbecco's Modification of Eagle's Medium (DMEM, Corning; 10 092 CM). The media was further supplemented with 10% fetal bovine serum (FBS, Corning; 35-011-CV), 100 μg/mL penicillin and 100 μg/mL streptomycin (Corning; 30-002-Cl), 100 μg/mL Normocin (Invivogen; ant-nr-05), 30 μg/mL Blasticidin (Invivogen; ant-b1-05), 100 μg/mL Zeocin (Invivogen; ant-zn-05), and 2 mM L-alanine L-glutamine (Glutagro, Corning; 25-015-Cl).

The small molecule control for hTLR8 assay was GS-9688, Selgantolimod; MedChem Express HY-109137, which is a known small molecule human TLR8 agonist. The oligo control for hTLR8 assay is poly U (Invivogen; tlrl-sspu). The competitive ASO control was ASO-1.

On the day of testing, LyoVec Transfection reagent (Invivogen; lyec-1) was prepared according to the manufacturer's protocol. In a 96-wel plate (Corning; 3997), 8 μL of prepared LyoVec was plated then 2 μL of each diluted ASO was added to the LyoVec solution in duplicate. HEK Blue hTLR8 cells were then added at the cell density of 80,000 cells per well in 190 μL of the assay media. The plates were then incubated at 37° C. and 5% CO₂ for 48 hours. At 48 hours post-transfection, 20 μL of the supernatant was harvested for secreted alkaline phosphatase assay (SEAP) using QUANTI-Blue Solution (Invivogen; rep-qbs) following the manufacturer's protocol. Optical density of the plates was read using a Perkin Elmer Envision instrument.

HEK Blue hTLR3 cells were obtained from Invivogen (Cat #hkb-htlr3). The assay protocol is the same as the protocol used for the HEK Blue hTLR8, except the assay control was Poly(A: U) (Invivogen; tlrl-pau).

HEK Blue hTLR4 (Invivogen; hkb-htlr4) were maintained in DMEM. The media was further supplemented with 10% FBS, 100 μg/mL penicillin and 100 μg/mL streptomycin, 100 μg/mL Normocin, 1×HEK Blue Selection (Invivogen; hb-sel), and 2 mM L-alanine L-glutamine. The assay protocol is the same as the protocol used for the HEK Blue hTLR8, except the assay control was purified lipopolysaccharide (LPS, Invivogen; tlrl-3pelps).

HEK Blue hTLR7 cells (Invivogen; hkb-htlr7) were maintained in DMEM. The media was further supplemented with 10% FBS, 100 μg/mL penicillin and 100 μg/mL streptomycin, 100 μg/mL Normocin, 30 μg/mL Blasticidin, 100 μg/mL Zeocin, and 2 mM L-alanine L-glutamine. The assay protocol is the same as the protocol used for the HEK Blue hTLR8, except the assay control was R848 (Resiquimod, Invivogen; tlrl-r848).

HEK Blue hTLR9 cells (Invivogen; hkb-htlr9) were maintained in the same growth media as that for HEK Blue hTLR7 cells. The assay protocol is the same as the protocol used for the HEK Blue hTLR8, except the assay control was ODN 2006 (ODN 7909, Invivogen; tlrl-2006). The effect of the disclosed ASOs on activating the various TLR pathways is shown in Table 3.

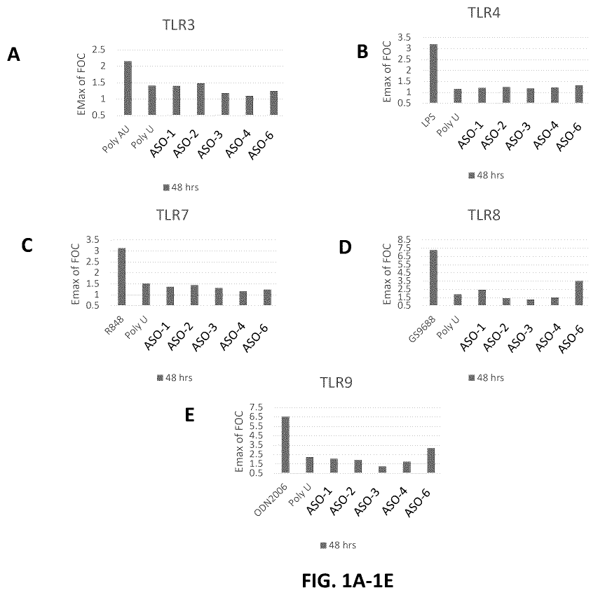

Activation of other TLR pathways, including TLR3, TLR4, TLR7, and TLR9 have been associated with immune response and inflammation, which on one hand might help combat infection, on the other hand may lead to increased negative side effects in the subject. To determine whether the control and parental ASOs activated TLR pathways other than hTLR8, the ASOs were subjected to the TLR3, TLR4, TLR7, and TLR9 activation assays as described above. ASO-1 activates human TLR8 more robustly than oligonucleotide positive control polyU; however, it did not activate human TLR3, TLR4, TLR7 and TLR9. ASO-6 activates human TLR8 more robustly than ASO-1. ASO-6 activates hTLR9 modestly but did not activate human TLR3, TLR4 and TLR7. ASOs ASO-2, ASO-3 and ASO-4 did not appear to activate any of the human TLRs in FIGS. 1A-1E. In summary, none of the ASOs activated the other TLR pathways as much as the positive activation control for each assay, except that ASO-6 modestly upregulated hTLR9. These data demonstrated that the control ASO-1 is a specific hTLR8 agonist, while ASO-6 is a stronger hTLR8 agonist than ASO-1 which also showed modest TLR9 agonist activity. The other ASOs tested showed no agonist activity in either hTLR3, hTLR4, hTLR7, hTLR8 or hTLR9 (FIGS. 1A-1E).

Finally, in order to determine whether the ASOs activate cell death mechanisms when transfected into cells, the ASOs were subjected to the caspase assay described below. The in vitro caspase 3/7 levels in multiple cell lines have been linked to necrosis in mouse livers in ASO toxicology studies. Lower caspase levels correlated with lower liver toxicity. The control and parental ASOs were transfected into cells at a concentration of 167 nM and 56 nM. The values of the parental ASOs were normalized to the level of caspase activity detected with the control ASO-1 sample. It was found that several parental ASOs, including ASO-4, ASO-318, ASO-6, and ASO-9, exhibited reduced levels of caspase activation as compared to the control ASO (Table 3, Column 5).

HepG2 cells were maintained in RPMI1640 medium with 10% fetal bovine serum (FBS) and 1% penicillin and 1% streptomycin. Cells were maintained at 37° C. in a 5% CO₂ atmosphere. The day before dosing, cells were seeded at a density that would reach 60% to 70% confluency in 24 hours. On the following day, ASOs were transfected at a concentration of 167 nM using Lipodectamine® RNAiMAX (Thermo Fisher, Cat #13778-150) following the manufacturer's instructions. The cells were incubated for 72 hours and then assayed for caspase induction using Promega's Caspase-Glo™ 3/7 Assay Kit (Cat #G8093) following manufacturer's instructions. The effect of the disclosed ASOs at a concentration 167 nM on activating caspases is shown in Table 3. For certain chemical modifications that are prone to increase liver toxicity, additional concentration of 56 nM was included in the caspase assay and analysis.

These assays indicate that the parental ASOs generated using the control ASO as a starting point for ASO development exhibit similar or enhanced activity as compared to the control ASO in several functional assays.

### Example 3: Generation of Secondary Modified ASOs

In order to generate additional ASOs, the ASO-6 parental ASO, which has higher hTLR8 activity and lower caspase activity than control ASO-1, was selected as the starting sequence for additional modifications. A series of modification types were selected for testing. The modifications include 2′ substituted nucleosides, including, but not limited to, 2′-MOE, 2′-O-cyp, 2′-O-mcyp, and 2′-OMe; 3′ substituted nucleosides, including, but not limited to 3′-xylo; 2′ and 3′ substituted nucleosides, including, but not limited to, 2′-OMe-3′-xylo; locked nucleosides, including, but not limited to, LNA, ScpBNA, AmNA, and GuNA; modified nucleobases, including, but not limited to, (8nh)G, (8nh)A, (2s)T, and (5oh)C; modified linkages, including phosphorothioate linkages and stereo-defined phosphorothioate linkages; and a combination of these various modifications. These modifications may increase the stability of the ASO while also increasing the ASO's abilities to reduce HBV infection. These modified nucleotides may be made via the methods described below or by standard methods known in the art.

The various modifications were introduced singly or in combination with other modifications to the ASOs and then subjected to the assays described above to determine RNAse H-mediated degradation, cytotoxicity, TLR8 pathway activation, and caspase activation (Table 4). A representative number of the test results of the various ASOs are shown in Table 3.

It was observed that modified ASOs with two or more LNAs exhibited increased caspase activation as compared to control ASOs lacking such modifications at 56 nM. In order to test whether the secondary modified ASOs activated caspases at this lower concentration, the caspase assay described above was repeated using a transfection concentration of 56 nM. A representative number of the caspase activity test results of the secondary modified ASOs at the lower concentration are shown in Table 4.

### Example 1 and 2 Results

TABLE 3
Assay Results for Tested ASOs

| | | | | Caspase |
|---|---|---|---|---|
| | | | hTLR8 Fold | Activity Fold |
| | | | Change vs. | Change vs. |
| ASO ID NO | EC₅₀ (nM) | CC₅₀ (nM) | Control | Control |
| ASO-1 | 8.6 | >167 | 1 | 1 |
| ASO-2 | 5.7 | >500 | 0.67 | 0.97 |
| ASO-3 | 5.8 | >500 | 0.48 | 1.1 |
| ASO-4 | 9.7 | >500 | 0.58 | 0.49 |
| ASO-318 | 5.9 | >500 | 0.56 | 0.90 |
| ASO-5 | 5.6 | >500 | 0.70 | 0.91 |
| ASO-6 | 5.8 | >167 | 1.5 | 0.86 |
| ASO-7 | 26 | >500 | 0.92 | 1.3 |
| ASO-319 | 8.0 | >500 | 0.82 | 1.5 |
| ASO-8 | 6.4 | >500 | 1.3 | 1.4 |
| ASO-9 | 5.6 | >500 | 1.4 | 0.93 |
| ASO-10 | 46.24 | >500 | 0.96 | N/A |
| ASO-11 | 54.80 | >500 | 0.48 | N/A |
| ASO-12 | 35.94 | >500 | 0.60 | N/A |
| ASO-13 | 29.44 | >500 | 067 | N/A |
| ASO-14 | 35.33 | >500 | 0.63 | N/A |
| ASO-15 | 28.25 | >500 | 1 | N/A |
| ASO-16 | >500 | >500 | 1.1 | N/A |
| ASO-17 | >167 | >167 | 0.41 | N/A |
| ASO-18 | 3.7 | >167 | 0.72 | 0.99 |
| ASO-19 | 3.2 | >167 | 0.79 | 1.0 |
| ASO-20 | 10.62 | >500 | 0.89 | N/A |
| ASO-21 | 9.28 | >500 | 0.98 | N/A |
| ASO-22 | 7.68 | >500 | 0.94 | N/A |
| ASO-23 | 5.90 | >500 | 0.96 | N/A |
| ASO-24 | 5.5 | 104.459 | N/A | N/A |
| ASO-25 | 6.4 | 151.324 | N/A | N/A |
| ASO-26 | 16 | 117.456 | N/A | N/A |
| ASO-27 | 5.6 | 148.018 | N/A | N/A |
| ASO-28 | 4.8 | 115.167 | N/A | N/A |
| ASO-29 | 4.3 | 137.998 | N/A | N/A |
| ASO-30 | 5.2 | 154.412 | N/A | N/A |
| ASO-31 | 5.2 | 122.362 | N/A | N/A |
| ASO-32 | 5.1 | 147.796 | N/A | N/A |
| ASO-33 | 4.5 | 158.769 | N/A | N/A |
| ASO-34 | 5.6 | 162.855 | N/A | N/A |
| ASO-35 | 6.93 | >500 | 0.96 | N/A |
| ASO-36 | 6.35 | >500 | 0.94 | N/A |
| ASO-37 | 3.56 | >500 | 0.92 | N/A |
| ASO-38 | 9.27 | 172.05 | 0.98 | N/A |
| ASO-40 | 5.43 | 155.97 | 0.82 | N/A |
| ASO-41 | 14 | >500 | 0.71 | N/A |
| ASO-42 | 17 | >500 | 0.75 | N/A |
| ASO-43 | 15 | >500 | 0.71 | N/A |
| ASO-44 | 17 | >500 | 0.76 | N/A |
| ASO-45 | 6.93 | 150.48 | 0.78 | N/A |
| ASO-46 | 4.28 | 165.65 | 0.99 | N/A |
| ASO-47 | 3.43 | 166.67 | 0.95 | N/A |
| ASO-48 | 6.66 | >500 | 0.98 | 0.96 |
| ASO-49 | 7.0 | >500 | 0.78 | 0.84 |
| ASO-50 | 11.02 | >500 | 0.46 | 0.81 |
| ASO-51 | 6.05 | >500 | 1.1 | 0.94 |
| ASO-52 | 9.54 | >500 | 0.82 | 0.56 |
| ASO-53 | 9.10 | >500 | 0.92 | 0.67 |
| ASO-54 | 5.55 | >500 | 0.98 | 0.90 |
| ASO-55 | 5.04 | >500 | 1.1 | 0.78 |
| ASO-56 | 18.31 | >500 | 1.2 | 0.79 |
| ASO-57 | N/A | N/A | 0.61 | N/A |
| ASO-58 | N/A | N/A | 0.63 | N/A |
| ASO-59 | N/A | N/A | 0.66 | N/A |
| ASO-60 | N/A | N/A | 0.77 | N/A |
| ASO-61 | N/A | N/A | 0.76 | N/A |
| ASO-62 | N/A | N/A | 0.71 | N/A |
| ASO-63 | N/A | N/A | 0.71 | N/A |
| ASO-64 | N/A | N/A | 0.64 | N/A |
| ASO-65 | N/A | N/A | 0.61 | N/A |
| ASO-66 | N/A | N/A | 0.86 | N/A |
| ASO-67 | 10.08 | >500 | 1.1 | N/A |
| ASO-68 | 11.76 | >500 | 0.67 | N/A |
| ASO-69 | 8.18 | >500 | 1.1 | N/A |
| ASO-70 | 9.17 | >500 | 1.0 | N/A |
| ASO-71 | 11.61 | >500 | 1.1 | N/A |
| ASO-72 | 9.33 | >500 | 1.1 | N/A |
| ASO-73 | 12.05 | >500 | 1.1 | N/A |
| ASO-74 | 8.08 | >500 | 1.0 | N/A |
| ASO-75 | 10.74 | >500 | 1.1 | N/A |
| ASO-76 | 11.78 | >500 | 1.1 | N/A |
| ASO-77 | 10.62 | >500 | 0.67 | N/A |
| ASO-78 | 9.8 | >500 | 0.72 | N/A |
| ASO-79 | 7.68 | >500 | 1.1 | N/A |
| ASO-80 | 5.71 | >500 | 1.1 | N/A |
| ASO-81 | 4.96 | >500 | 1.0 | N/A |
| ASO-82 | 8.16 | >500 | 1.2 | N/A |
| ASO-83 | 5.28 | >500 | 1.1 | N/A |
| ASO-84 | 9.94 | >500 | 1.0 | N/A |
| ASO-85 | 8.03 | >500 | 1.1 | N/A |
| ASO-86 | 7.66 | >500 | 1.1 | N/A |
| ASO-87 | 5.77 | >500 | 1.1 | N/A |
| ASO-88 | 6.63 | >500 | 1.1 | N/A |
| ASO-89 | 6.72 | >500 | 1.1 | N/A |
| ASO-90 | 9.76 | >500 | 1.0 | 0.88 |
| ASO-91 | 7.20 | >500 | 0.62 | 1.1 |
| ASO-92 | 8.61 | >500 | 1.0 | 1.1 |
| ASO-93 | 9.58 | >500 | 0.49 | 0.93 |
| ASO-94 | 8.41 | >500 | 0.82 | 0.92 |
| ASO-95 | 8.23 | >500 | 0.49 | 1.0 |
| ASO-96 | 11.25 | >500 | 0.71 | 0.99 |
| ASO-97 | 8.27 | >500 | 0.93 | 1.1 |
| ASO-98 | 12.46 | >500 | 0.84 | 1.1 |
| ASO-99 | 10.76 | >500 | 1.3 | 1.1 |
| ASO-100 | 10.62 | >500 | 0.98 | 0.91 |
| ASO-101 | 8.91 | >500 | 1.1 | 1.1 |
| ASO-102 | 7.25 | >500 | 0.95 | 1.1 |
| ASO-103 | 6.603 | >500 | 0.83 | 1.2 |
| ASO-104 | 6.69 | >500 | 0.89 | 1.1 |
| ASO-105 | 8.93 | >500 | 0.84 | 1.2 |
| ASO-106 | 11.98 | >500 | 0.83 | 1.0 |
| ASO-107 | 6.57 | >500 | 0.94 | 1.1 |
| ASO-108 | 6.41 | >500 | 0.84 | 0.61 |
| ASO-109 | 7.83 | >500 | 1.4 | 1.0 |
| ASO-110 | 7.075 | >500 | 1.3 | 0.76 |
| ASO-111 | 7.47 | >500 | 1.2 | 0.73 |
| ASO-112 | 7.67 | >500 | 1.3 | 0.72 |
| ASO-113 | 9.03 | >500 | 1.5 | 0.74 |
| ASO-114 | 6.18 | >500 | 1.7 | 0.73 |
| ASO-115 | 7.88 | >500 | 1.3 | 0.75 |
| ASO-116 | 8.81 | >500 | 1.5 | 0.85 |
| ASO-117 | 7.71 | >500 | 1.2 | 0.80 |
| ASO-118 | 3.87 | >500 | 0.99 | 0.91 |
| ASO-121 | 8.26 | >500 | 0.72 | 0.61 |
| ASO-122 | 7.70 | >500 | 1.1 | 0.82 |
| ASO-123 | 6.59 | >500 | 1.2 | 0.95 |
| ASO-124 | 3.08 | >500 | 1.2 | 0.91 |
| ASO-125 | 5.40 | >500 | 1.1 | 0.90 |
| ASO-126 | 5.55 | >500 | 1.2 | 0.86 |
| ASO-127 | 1.88 | >500 | 1.1 | 0.95 |
| ASO-128 | 7.76 | >500 | 1.06 | 0.92 |
| ASO-129 | 3.96 | >500 | 1.03 | 0.96 |
| ASO-130 | 5.27 | 158.1 | 0.92 | 1.04 |
| ASO-131 | 3.36 | >500 | 0.90 | 0.96 |
| ASO-132 | 3.22 | 158.13 | 0.94 | 0.997 |
| ASO-133 | 3.92 | >500 | 0.96 | 1.04 |
| ASO-134 | 5.08 | >500 | 0.85 | 0.787 |
| ASO-135 | 3.94 | 173.9 | 0.88 | 0.843 |
| ASO-136 | 0.69 | >500 | 0.81 | 0.756 |
| ASO-137 | 6.93 | >500 | 0.91 | 0.739 |
| ASO-138 | 4.91 | >500 | 0.98 | 0.90 |
| ASO-140 | 11.46 | >500 | 1.18 | 0.95 |
| ASO-141 | 13.45 | >500 | 1.06 | 1.1 |
| ASO-142 | 7.17 | >500 | 1.00 | 1.1 |
| ASO-143 | 8.73 | >500 | 0.75 | 1.1 |
| ASO-144 | 12.39 | >500 | 1.05 | 0.95 |
| ASO-145 | 9.29 | >500 | 1.30 | 1.0 |
| ASO-146 | 9.91 | >500 | 1.05 | 1.1 |
| ASO-147 | 9.61 | >500 | 1.06 | 1.1 |
| ASO-148 | 7.40 | >500 | 1.14 | 1.1 |
| ASO-149 | 9.84 | >500 | 1.14 | 1.0 |
| ASO-150 | 6.68 | >500 | 1.04 | 1.1 |
| ASO-151 | 9.59 | >500 | 1.38 | 0.85 |
| ASO-152 | 9.69 | >500 | 1.43 | 1.1 |
| ASO-153 | 10.39 | >500 | 1.73 | 1.1 |
| ASO-154 | 7.83 | >500 | 1.33 | 1.045 |
| ASO-155 | 7.08 | >500 | 1.49 | 1.038 |
| ASO-156 | 7.47 | 484.39 | 1.24 | 1.064 |
| ASO-157 | 7.67 | >500 | 1.34 | 0.879 |
| ASO-158 | 9.03 | >500 | 1.32 | 0.858 |
| ASO-159 | 6.18 | >500 | 1.37 | 0.940 |
| ASO-160 | 7.88 | >500 | 1.34 | 0.933 |
| ASO-161 | 8.81 | >500 | 1.13 | 1.033 |
| ASO-162 | 7.71 | >500 | 0.89 | 0.944 |
| ASO-163 | 3.87 | >500 | 1.20 | 0.816 |
| ASO-164 | 8.76 | >500 | 1.2 | 1.1 |
| ASO-165 | 6.90 | >500 | 1.2 | 1.1 |
| ASO-166 | 8.27 | >500 | 1.2 | 1.1 |
| ASO-167 | 13.23 | >500 | 1.1 | 1.1 |
| ASO-168 | 7.87 | >500 | 0.92 | 1.3 |
| ASO-170 | 4.53 | >500 | 1.3 | 1.1 |
| ASO-171 | 5.32 | >500 | 1.5 | 1.1 |
| ASO-172 | 6.81 | >500 | 1.2 | 1.2 |
| ASO-173 | 6.78 | >500 | 1.3 | 1.1 |
| ASO-174 | 2.21 | >500 | 1.36 | 1.2 |
| ASO-175 | 3.41 | >500 | 1.23 | 1.1 |
| ASO-176 | 4.83 | >500 | 1.60 | 1.3 |
| ASO-177 | 4.98 | >500 | 1.52 | 1.2 |
| ASO-178 | 4.71 | >500 | 1.50 | 1.2 |
| ASO-179 | 3.97 | 179.33 | 1.20 | 1.3 |
| ASO-180 | 3.35 | >500 | 1.32 | 1.2 |
| ASO-181 | 6.50 | >500 | 1.38 | 1.2 |
| ASO-182 | 3.36 | >500 | 1.52 | 1.2 |
| ASO-183 | 2.62 | >500 | 1.02 | 1.3 |
| ASO-184 | 3.00 | >500 | 1.11 | 1.1 |
| ASO-209 | 2.26 | 105.7 | 1.6 | 1.1 |
| ASO-210 | 3.105 | 83.43 | 1.5 | 1.1 |
| ASO-211 | 3.98 | 97.68 | 1.7 | 1.1 |
| ASO-212 | 3.21 | 105.5 | 1.6 | 1.2 |
| ASO-213 | 3.06 | 102.9 | 1.5 | 1.2 |
| ASO-214 | 2.66 | >500 | 1.2 | 1.2 |
| ASO-215 | 2.53 | 126.7 | 1.8 | 0.94 |
| ASO-216 | 3.61 | 131.8 | 1.8 | 0.99 |
| ASO-217 | 3.24 | 123.9 | 1.7 | 1.1 |
| ASO-218 | 3.77 | 176.2 | 1.3 | 1.1 |
| ASO-219 | 2.5 | 184.0 | 1.2 | 1.5 |
| ASO-220 | 0.90 | 55.4 | 1.4 | 1.6 |
| ASO-221 | 1.90 | 143.6 | 1.5 | 1.6 |
| ASO-222 | 1.23 | >500 | 1.5 | 1.0 |

TABLE 4
Caspase Activation for ASOs with Two or More LNAs at 56 nM

| | Caspase Activity |
|---|---|
| | Fold Change vs. |
| ASO ID NO. | Control |
| ASO-1 | 1 |
| ASO-174 | 1.7 |
| ASO-175 | 1.5 |
| ASO-176 | 1.5 |
| ASO-177 | 1.2 |
| ASO-178 | 1.1 |
| ASO-179 | 1.8 |
| ASO-180 | 1.1 |
| ASO-181 | 1.1 |
| ASO-182 | 1.1 |
| ASO-183 | 1.6 |
| ASO-184 | 1.8 |
| ASO-209 | 1.3 |
| ASO-210 | 1.2 |
| ASO-211 | 1.2 |
| ASO-212 | 1.2 |
| ASO-213 | 1.1 |
| ASO-214 | 1.8 |
| ASO-215 | 1.1 |
| ASO-216 | 1.0 |
| ASO-217 | 0.94 |
| ASO-218 | 1.2 |
| ASO-219 | 1.5 |
| ASO-220 | 2.0 |
| ASO-221 | 1.9 |
| ASO-222 | 1.2 |

### Example 4: Preparation of Key Intermediate 6S-1 & 6R-1 for Example 5 and 6 Dimers

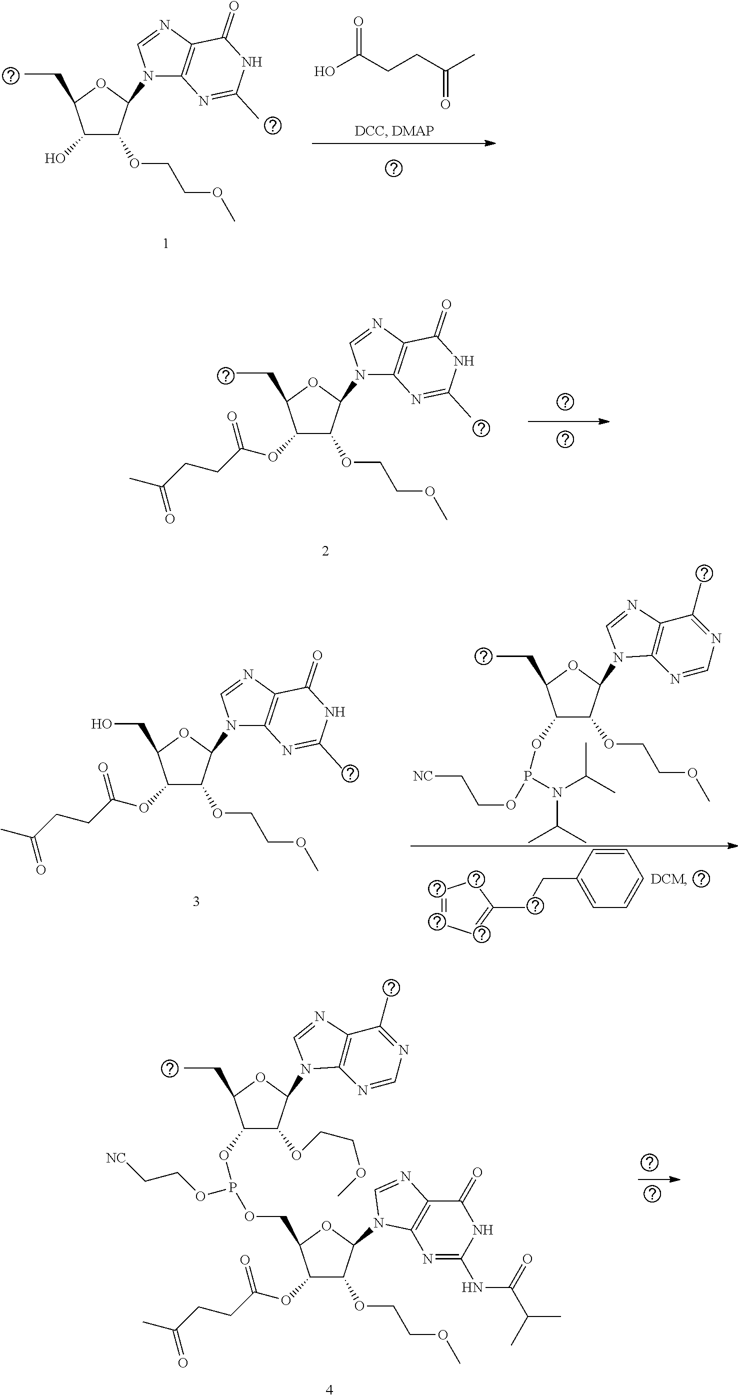

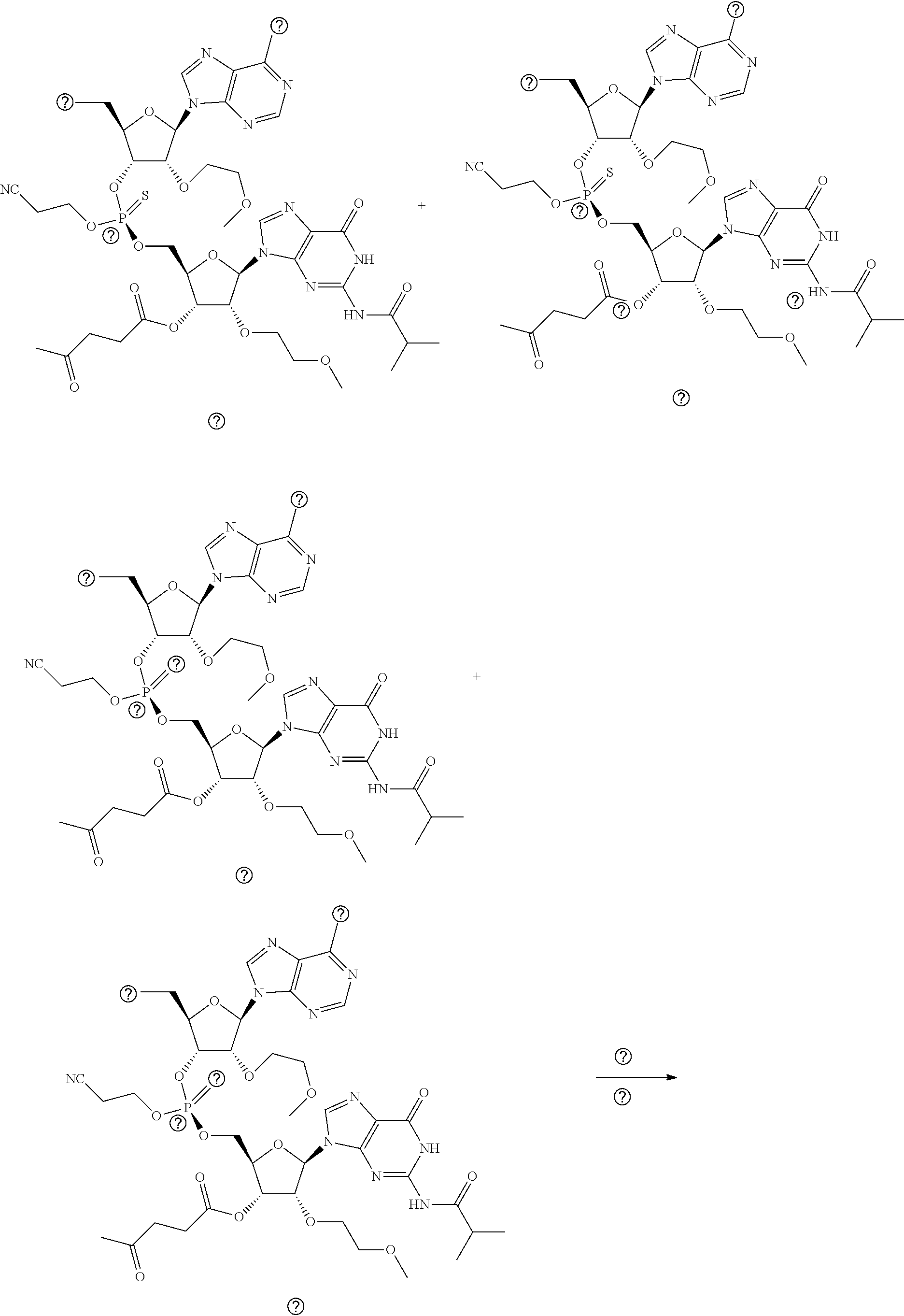

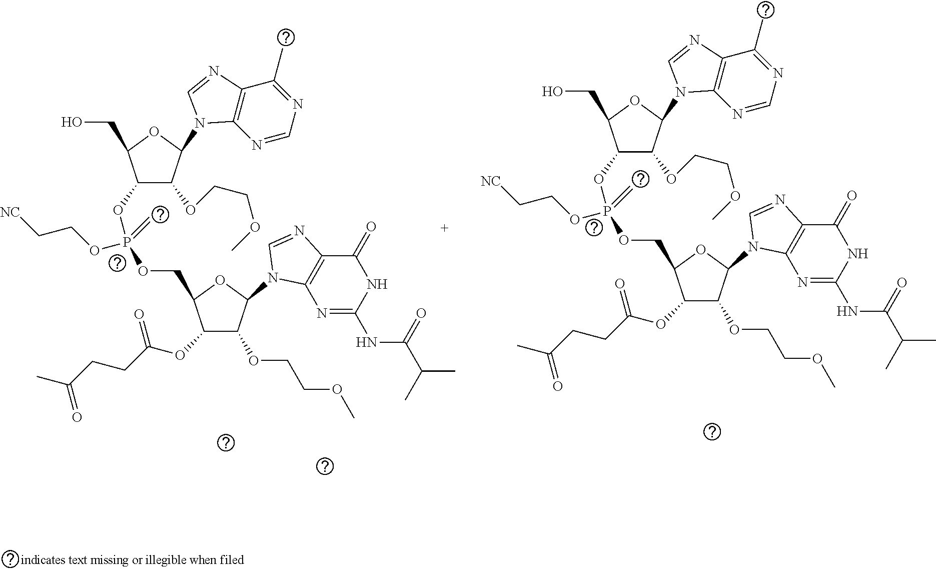 [SMILES: C=[P@@](OCCC#N)(OC[C@H]1O[C@@H](n2cnc3c(=O)[nH]c(NC(=O)C(C)C)nc32)[C@H](OCCOC)[C@@H]1OC(=O)CCC(C)=O)O[C@H]1[C@@H](OCCOC)[C@H](n2cnc3c(C)ncnc32)O[C@@H]1CO ; C=[P@@](OCCC#N)(OC[C@H]1O[C@@H](n2cnc3c(=O)[nH]c(NC(=O)C(C)C)nc32)[C@H](OCCOC)[C@@H]1OC(=O)CCC(C)=O)O[C@H]1[C@@H](OCCOC)[C@H](n2cnc3c(C)ncnc32)O[C@@H]1CO]

Preparation of (2): To a solution of 1 (50.0 g, 71.8 mmol) in dioxane (500 mL) was added DCC (22.8 g, 111.1 mmol) and DMAP (4.5 g, 37.0 mmol). Then added Lev acid (52.2 g, 442.8 mmol) at 0° C. and the reaction mixture was stirred at room temperature for 1 hour. LCMS showed 1 was consumed completely. The reaction mixture was quenched by NaHCO₃ and extraction with DCM. And the organic phase was washed with water and saturated brine and dried over by Na₂SO₄. Then the solution was concentrated under reduced pressure. This resulted in 3 (59.0 g, crude) as solid which was used directly for the next step. ESI-LCMS: m/z 810.2 [M+H]+.

Preparation of (3): To a solution of 2 (57.0 g) was dissolved 6% of DCA in DCM (1.7 L) was added TES (17.0 g, 146.0 mmol) at room temperature (r.t.). The mixture was stirred at rt. for 10-20 min. LCMS and TLC show 2 was completely consumed. The reaction was quenched by the addition of sat. NaHCO₃ (aq.). The resulting mixture was extracted with DCM. Then the combined organic layers were washed the organic phase once with saturated brine and dried over by Na₂SO₄. Then the solution was concentrated under reduced pressure. This resulted was purified by silica gel column chromatograph (eluent, PE:EA=3:1-1:2). This resulted in 3 (26.0 g, 51.1 mmol) as a white solid. ESI-LCMS: m/z 828.2 [M−H]⁻

Preparation of (4): To a solution of 3 (24.0 g, 47.1 mmol) and 3a (46.0 g, 49.5 mmol) in ACN (240 mL) was added molecular sieve and was stirred at room temperature for 15 minutes. Then the mixture was added BTT (0.3 M, 235.5 mL) and stirred at room temperature for 0.5 hour. LCMS showed 3 was consumed completely. The mixture used for directly.

Preparation of (5R & 5S): The mixture was added pyridine and xanthane hydride (14.1 g, 94.2 mmol) then was stirred at room temperature for 15 minutes. After filter, the mixture was extracted with EA, washed with water and brine then dried over by anhydrous Na₂SO₄. The mixture was concentrated to give the crude (52.0 g 37.9 mmol). The mixture used for directly. ESI-LCMS: m/z 1370.2 [M−H]⁻. ³¹P-NMR (400 MHZ, DMSO-d6): δ 67.12, 66.94.

Preparation of (6R-1 & 6S-1): To a solution of 5 (23.0 52.0 g, 16.7 mmol) was dissolved 6% of DCA in DCM (230 mL) was added TES (2.9 g, 20.0 mmol) at r.t. The mixture was stirred at rt. for 10-20 min. LCMS and TLC show 5 was completely consumed. The reaction was quenched by the addition of sat. NaHCO₃ (aq.). The resulting mixture was extracted with DCM. Then the combined organic layers were washed the organic phase once with saturated brine and dried over by Na₂SO₄. Then washed the organic phase once with saturated brine and dried over by Na₂SO₄. Then the solution was concentrated under reduced pressure. The residue was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=1/0 within 25 min, the eluted product was collected at CH₃CN/H₂O (0.5% NH₄HCO₃)=2/1; Detector, UV 254 nm. This resulted was purified by SFC. This resulted in 6R-1 (5.5 g, 4.6 mmol) and 6S-1 (5.0 g, 5.1 mmol) as a white solid. ESI-LCMS: m/z 1070.2 [M+H]⁻; ³¹P-NMR (400 MHZ, DMSO-d6): δ 66.90, 66.82.

### Example 5: Preparation of Example 5 Dimer from Intermediate 6S-1

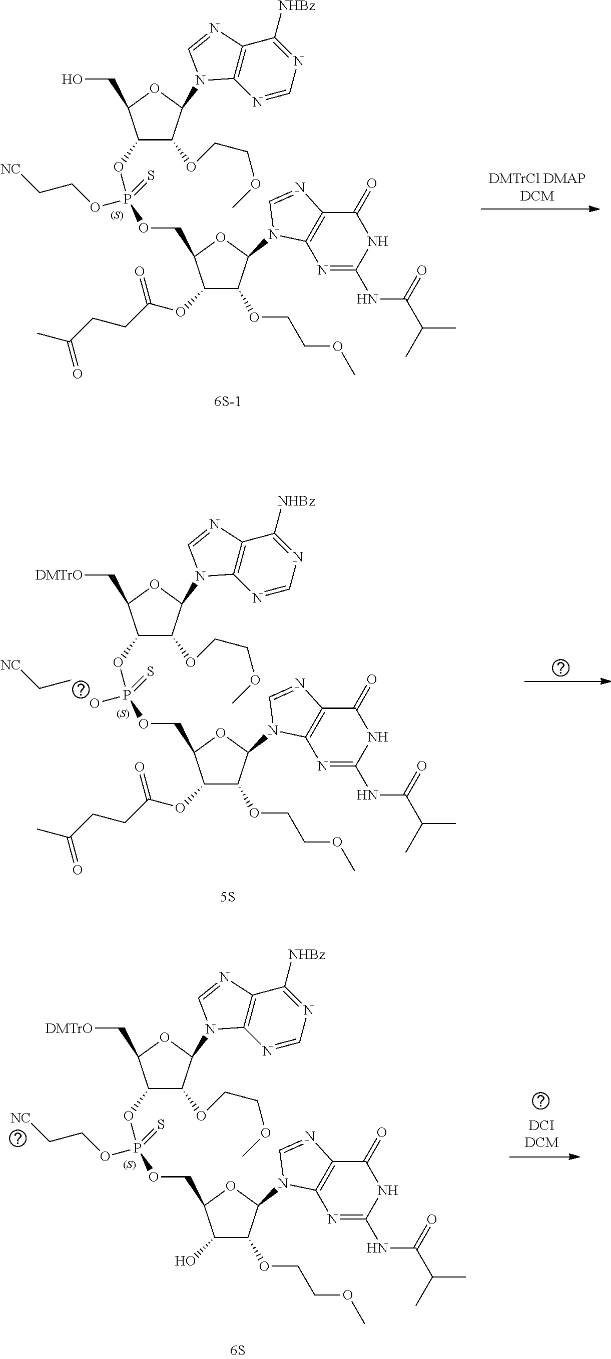

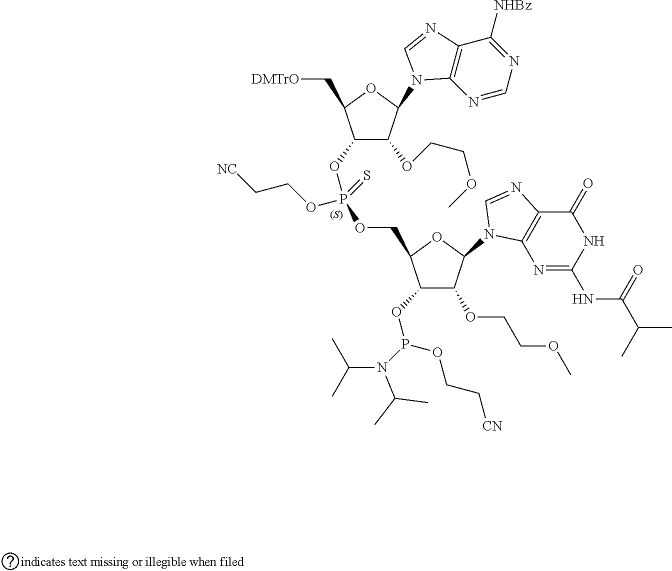

Preparation of (5S): To a stirred mixture of 6S-1 (4.8 g, 4.4 mmol) in pyridine (48 mL) was added DMAP (105 mg, 0.89 mmol) and DMTrCl (1.9 g, 5.71 mmol) at r.t under N₂ atmosphere. The resulting mixture was stirred at r.t under argon atmosphere for 24 h. LCMS and TLC show SM was completely consumed. The reaction was quenched by the addition of sat. NaHCO₃ (aq.). The resulting mixture was extracted with EA. And the organic phase was washed with water and saturated brine and dried over by Na₂SO₄. Then the solution was concentrated under reduced pressure. This resulted in crude 5S (6.0 g) as a solid used directly next step. ESI-LCMS: m/z 1370.2 [M−H].

Preparation of (6S): To a solution of 7S (6.0 g, 4.3 mmol) in ACN (60 mL) was added N2H₄ (0.5M, 43 mL) at 0° C. The reaction was stirred at 0° C. for 0.5 hour. LCMS showed 7S was consumed completely. The reaction mixture was quenched by 2,4-pentanedione at 0° C. for 15 minutes and extraction with EA. The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/1; Detector, UV 254 nm. This resulted in 6S (3.3 g, 87.5% yield) as a white solid. ESI-LCMS: m/z 1272.2 [M−H]⁻; ¹H NMR (400 MHZ, DMSO-d₆) δ 12.11 (s, 1H), 11.65 (s, 1H), 11.26 (s, 1H), 8.61 (dt, J=19.2, 1.5 Hz, 2H), 8.22 (t, J=1.7 Hz, 1H), 8.17-7.97 (m, 2H), 7.64 (t, J=7.4 Hz, 1H), 7.55 (t, J=7.6 Hz, 2H), 7.48-7.34 (m, 2H), 7.33-7.21 (m, 8H), 6.84 (d, J=8.5 Hz, 4H), 6.20 (dt, J=6.9, 2.0 Hz, 1H), 5.98 (dt, J=5.8, 2.0 Hz, 1H), 5.43 (dt, J=5.3, 1.4 Hz, 1H), 5.41-5.30 (m, 1H), 4.55-4.37 (m, 3H), 4.29 (q, J=7.6, 6.6 Hz, 4H), 4.21-4.14 (m, 1H), 3.60 (ddd, J=22.4, 10.9, 5.8 Hz, 2H), 3.42 (t, J=4.8 Hz, 2H), 3.39-3.24 (m, 4H), 3.17 (s, 3H), 3.03 (t, J=1.1 Hz, 3H), 2.94 (t, J=5.9 Hz, 2H), 2.77 (h, J=7.0 Hz, 1H), 1.10 (dd, J=13.5, 6.7 Hz, 6H).

Preparation of Example 5 Dimer: To a solution of 6S (3.0 g, 2.0 mmol) in DCM (30 mL) was added DCI (200 mg, 1.7 mmol) and CEP[N(iPr)₂]₂ (842 mg, 2.8 mmol) at r.t at N₂. The mixture was stirred at r.t at N₂ for 2 h. LC-MS showed all precursor was consumed completely. The mixture was added NaHCO₃ aqueous (30 mL) and extracted with DCM. The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in Example 5 dimer (2.0 g, 1.3 mmol, 74% yield,) as a white solid. ESI-LCMS: m/z 1472.2 [M−H]^{- 1}H NMR (600 MHZ, DMSO-d₆) δ 11.62 (s, 1H), 8.72-8.47 (m, 2H), 8.32-7.99 (m, 3H), 7.69-7.49 (m, 3H), 7.46-7.18 (m, 9H), 6.95-6.78 (m, 4H), 6.21 (s, 1H), 6.15-5.82 (m, 1H), 5.52-5.05 (m, 2H), 4.84-4.51 (m, 2H), 4.49-4.09 (m, 6H), 3.92-3.49 (m, 14H), 3.45-3.35 (m, 3H), 3.29-3.21 (m, 2H), 3.20-3.10 (m, 3H), 3.01-2.86 (m, 5H), 2.80 (dt, J=30.9, 6.2 Hz, 3H), 1.25-1.08 (m, 19H). ³¹P NMR (243 MHz, DMSO-d₆) δ 150.26, 149.87, 66.96.

### Example 6: Preparation of Example 6 Dimer from Intermediate 6R-1

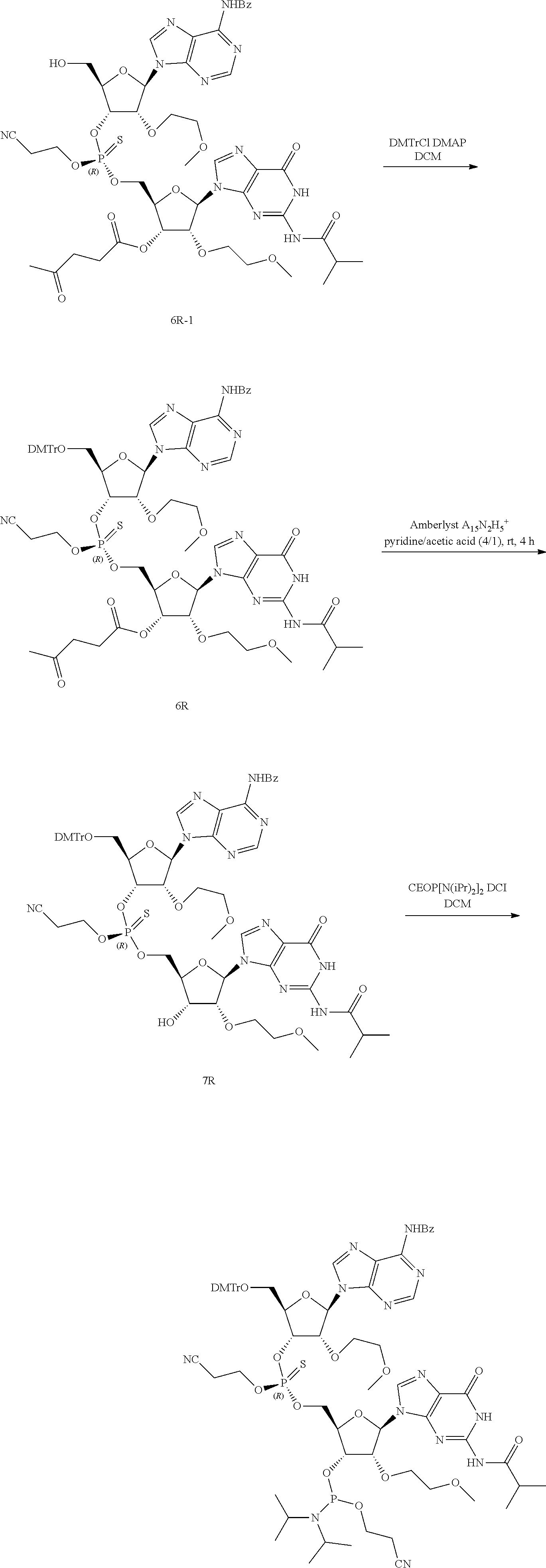

Preparation of (5R): To a stirred mixture of 6R-1 (4.8 g, 4.4 mmol) in pyridine (48 mL) was added DMAP (105 mg, 0.89 mmol) and DMTrCl (1.9 g, 5.71 mmol) at r.t under N₂ atmosphere. The resulting mixture was stirred at r.t under argon atmosphere for 24 h. LCMS and TLC show SM was completely consumed. The reaction was quenched by the addition of sat. NaHCO₃ (aq.). The resulting mixture was extracted with EA. The reaction was quenched by the addition of sat. NaHCO₃ (aq.). The resulting mixture was extracted with EA. And the organic phase was washed with water and saturated brine and dried over by Na₂SO₄. Then the solution was concentrated under reduced pressure. This resulted in crude 5R (6.0 g) as a solid used directly next step. ESI-LCMS: m/z 1370.2 [M−H]⁻.

Preparation of (6R): To a solution of 5R (4.7 g, 3.7 mmol) in ACN (47.0 mL) was added N₂H₄ (0.5M, 37.0 mL) at 0° C. The reaction was stirred at 0° C. for 0.5 hour. LCMS showed 5R was consumed completely. The reaction mixture was quenched by 2,4-pentanedione at 0° C. for 15 minutes and extraction with EA. The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/1; Detector, UV 254 nm. This resulted in 6R (3.0 g, 2.0 mmol, 65% yield) as a white solid. ESI-LCMS: m/z 1272.2 [M−H]⁻; ¹H NMR (400 MHZ, DMSO-d₆) δ 12.11 (s, 1H), 11.65 (s, 1H), 11.26 (s, 1H), 8.61 (dt, J=19.2, 1.5 Hz, 2H), 8.22 (t, J=1.7 Hz, 3H), 8.13-7.90 (m, 2H), 7.64 (t, J=7.4 Hz, 1H), 7.55 (t, J=7.6 Hz, 2H), 7.44-7.33 (m, 2H), 7.33-7.01 (m, 12H), 6.84 (d, J=8.5 Hz, 4H), 6.20 (dt, J=6.9, 2.0 Hz, 1H), 5.98 (dt, J=5.8, 2.0 Hz, 1H), 5.43 (dt, J=5.3, 1.4 Hz, 1H), 5.41-5.31 (m, 1H), 4.58-4.38 (m, 3H), 4.29 (q, J=7.6, 6.6 Hz, 4H), 4.22-4.12 (m, 1H), 3.60 (ddd, J=22.4, 10.9, 5.8 Hz, 2H), 3.42 (t, J=4.8 Hz, 3H), 3.17 (s, 3H), 3.03 (t, J=1.1 Hz, 3H), 2.94 (t, J=5.9 Hz, 2H), 2.77 (h, J=7.0 Hz, 1H), 2.29 (s, 3H), 1.10 (dd, J=13.5, 6.7 Hz, 6H). ³¹P NMR: δ 67.14.

Preparation of Example 6 Dimer: To a solution of 6R (3.0 g, 2.4 mmol) in DCM (30.0 mL) with an inert atmosphere of nitrogen was added CEOP[N(iPr)₂]₂ (933.1 mg, 3.1 mmol) and DCI (240.7 mg, 2.1 mmol) in order at room temperature. The resulting solution was stirred for 1.0 hour at room temperature and diluted with 50.0 mL dichloromethane and washed with 2×5.0 mL of saturated aqueous sodium bicarbonate and 1×50.0 mL of saturated aqueous sodium chloride respectively. The organic phase was dried over anhydrous sodium sulfate, filtered and concentrated till no residual solvent left under reduced pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=9/1; Detector, UV 254 nm. This resulted in Example 6 dimer (1.9 g, 1.3 mmol, 56% yield) as yellow solid. ESI-LCMS: m/z 1472.2 [M−H]⁻; ¹H-NMR (400 MHZ, DMSO-d₆): δ11.31-11.12 (m, 2H), 8.70-8.58 (m, 3H), 8.05-8.00 (m, 4H), 7.98-7.26 (m, 15H), 7.26-7.25 (m, 1H), 6.91-6.88 (m, 4H), 6.62-6.59 (m, 1H), 6.01-5.76 (m, 2H), 5.24-5.50 (m, 2H), 4.70-4.00 (m, 6H), 3.98-3.82 (2H), 3.73-3.72 (m, 6H), 3.45-3.33 (m, 4H), 1.20-1.16 (m, 12H); ³¹P-NMR (162 MHZ, DMSO-d6): δ 150.16, 149.92, 66.83, 66.74.

### Example 7: Preparation of Key Intermediate 6S and 6R for Example 8 and Example 9 Dimers

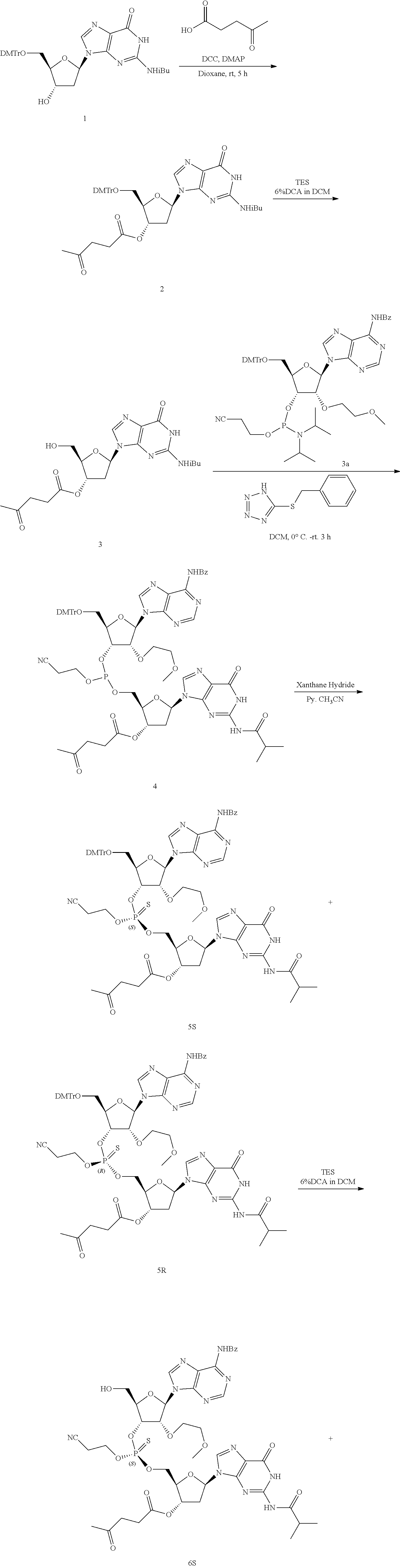

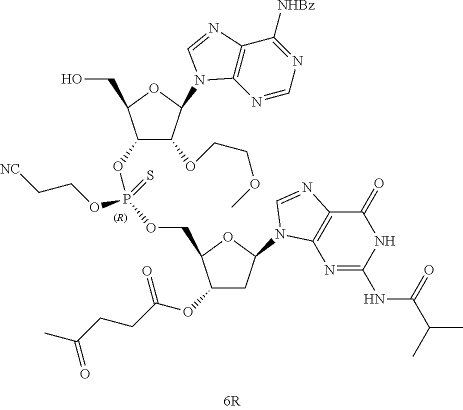

Preparation of (2): To a solution of 1 (48.0 g, 75.0 mmol) in dioxane (480 mL) was added DCC (23.2 g, 112.5 mmol) and DMAP (4.2 g, 34.5 mmol). Then added Lev acid (52.2 g, 450.0 mmol) at 0° C. and the reaction mixture was stirred at room temperature for 1 hour. LCMS showed 1 was consumed completely. The reaction mixture was quenched by NaHCO₃ and extraction with DCM. And the organic phase was washed with water and saturated brine and dried over by Na₂SO₄. Then the solution was concentrated under reduced pressure. This resulted in 3 (45.3 g, crude) as solid which was used directly for the next step. ESI-LCMS: m/z 736.3 [M+H]+.

Preparation of (3): To a solution of 2 (45.3 g, 61.5 mmol) was dissolved 6% of DCA in DCM (450 mL) was added TES (10.7 g, 92.3 mmol) at r.t. The mixture was stirred at rt. for 10-20 min. LCMS and TLC show 2 was completely consumed. The reaction was quenched by the addition of sat. NaHCO₃ (aq.). The resulting mixture was extracted with DCM. And the organic phase was washed with water and saturated brine and dried over by Na₂SO₄. Then the solution was concentrated under reduced pressure. The residue was purified by silica gel column chromatograph (eluent, PE/EA=3:1-1:2). This resulted in 3 (21.4 g, 80.0% yield) as a white solid. ESI-LCMS: m/z 436.1 [M+H]+

Preparation of (4): To a solution of 3 (21.4 g, 49.2 mmol) and 3a (41.7 g, 44.7 mmol) in ACN (420.0 mL) was added molecular sieve and was stirred at room temperature for 15 minutes. Then the mixture was added BTT (0.3 M, 201.3 mL) and stirred at room temperature for 0.5 hour. LCMS showed 3 was consumed completely. The mixture used for directly.

Preparation of (5R & 5S): The mixture was added pyridine and xanthane hydride (10.1 g, 67.2 mmol) then was stirred at room temperature for 15 minutes. After filter, the mixture was extracted with EA. And the organic phase was washed with water and saturated brine and dried over by Na₂SO₄. Then the solution was concentrated under reduced pressure. This resulted in the crude 5R&5S (20.0 g). The mixture used for directly; ESI-LCMS: m/z 1298.4 [M+H]+.

Preparation of (6R & 6S): To a solution of 5R & 5S (15.0 g, 11.6 mmol) was dissolved 6% of DCA in DCM (150 mL) was added TES (1.3 g, 17.4 mmol) at r.t. The mixture was stirred at rt. for 10-20 min. LCMS and TLC show SM was completely consumed. The reaction was quenched by the addition of sat. NaHCO₃ (aq.). The resulting mixture was extracted with DCM. Then washed the organic phase once with saturated brine and dried over by Na₂SO₄. Then the solution was concentrated under reduced pressure. The residue was purified by silica gel column chromatography (eluent, PE:EA=2: 1˜1:1). The residue was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=1/0 within 25 min, the eluted product was collected at CH₃CN/H₂O (0.5% NH₄HCO₃)=8/1; Detector, UV 254 nm. This resulted in 6R (4.5 g) and 6S (6.4 g) as a white solid. ESI-LCMS: m/z 996.1 [M+H]+; 1H-NMR (400 MHZ, DMSO-d6): δ 11.31-11.17 (m, 2H), 8.73-8.56 (m, 3H), 8.05-8.00 (m, 4H), 7.98-7.28 (m, 16H), 7.26-7.25 (m, 1H), 6.91-6.87 (m, 4H), 6.62-6.59 (m, 1H), 6.01-5.79 (m, 3H), 5.21-5.02 (m, 1H), 4.70-4.00 (m, 6H), 3.74-3.72 (m, 6H), 3.50-3.34 (m, 4H), 2.78-2.50 (m, 6H), 2.12-2.08 (m, 3H); 31P-NMR (162 MHz, DMSO-d6): δ 68.37, 67.91, 66.82, 66.47.

### Example 8: Preparation of Example 8 Dimer from 6S

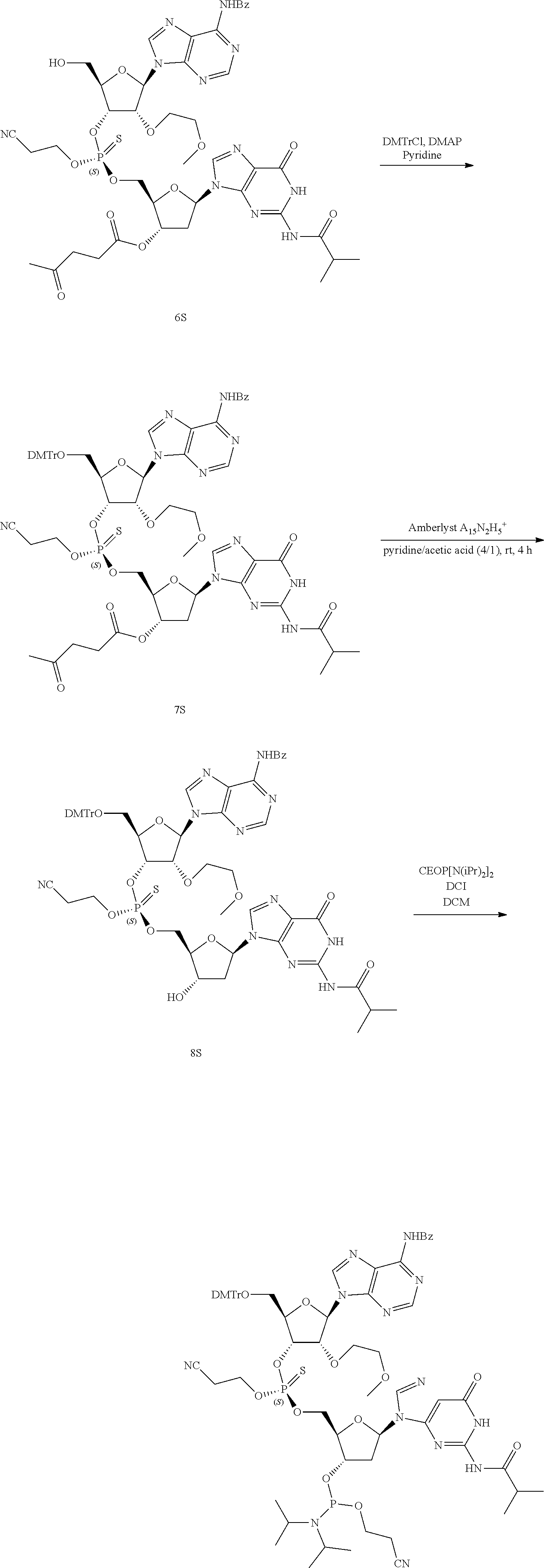

Preparation of (7S): To a stirred mixture of 6S (6.4 g, 6.4 mmol) in pyridine (65 mL) was added DMAP (156 mg, 12.8 mmol) and DMTrCl (4.3 g, 12.8 mmol) at r.t under N₂ atmosphere. The resulting mixture was stirred at r.t under argon atmosphere for 24 h. LCMS and TLC show SM was completely consumed. The reaction was quenched by the addition of sat. NaHCO₃ (aq.). The resulting mixture was extracted with EA. And the organic phase was washed with water and saturated brine and dried over by Na₂SO₄. Then the solution was concentrated under reduced pressure. The residue was purified by silica gel column chromatograph (eluent, DCM (0.5% pyridine) THF=1:0-0:1). This resulted in 7S (22.0 g) as a white solid. ESI-LCMS m/z 1298.0 [M+H]⁺.

Preparation of (8S): To a solution of 7S (4.5 g, 3.5 mmol) in ACN (35 mL) was added N₂H₄ (0.5M, 17.5 ml) at 0° C. The reaction was stirred at 0° C. for 0.5 hour. LCMS showed 7S was consumed completely. The reaction mixture was quenched by 2,4-pentanedione at 0° C. for 15 minutes and extraction with EA. The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/1; Detector, UV 254 nm. This resulted in giving 8S (3.0 g, 72% yield) as a white solid. ESI-LCMS: m/z 1200.4 [M+H]+; 1H-NMR (400 MHZ, DMSO-d6): δ 11.31-11.12 (m, 2H), 8.73-8.52 (m, 3H), 8.04-8.03 (m, 4H), 7.99-7.26 (m, 15H), 7.26-7.25 (m, 1H), 6.91-6.87 (m, 4H), 6.62-6.59 (m, 1H), 6.01-5.79 (m, 2H), 5.68-5.56 (m, 1H), 5.21-5.02 (m, 1H), 4.70-4.00 (m, 6H), 3.73-3.72 (m, 6H), 3.50-3.45 (m, 4H), 2.90-2.80 (m, 2H); 31P-NMR (162 MHZ, DMSO-d6): δ 66.83, 66.58.

Preparation of Example 8 Dimer: To a solution of 8S (3.0 g, 2.5 mmol) in DCM (30 mL) with an inert atmosphere of nitrogen was added CEP[N(iPr)₂]₂ (979.6 mg, 3.2 mmol) and DCI (251 mg, 2.1 mmol) in order at room temperature. The resulting solution was stirred for 1.0 hour at room temperature and diluted with 30.0 mL dichloromethane and the organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in Example 8 dimer (2.0 g, 56% yield) as yellow solid. ESI-LCMS: m/z 1368.2 [M−H]−; 1H-NMR (400 MHZ, DMSO-d6): δ11.31-11.12 (m, 2H), 8.70-8.58 (m, 3H), 8.05-8.00 (m, 4H), 7.98-7.26 (m, 15H), 7.26-7.25 (m, 1H), 6.91-6.88 (m, 4H), 6.62-6.59 (m, 1H), 6.01-5.76 (m, 2H), 5.24-5.50 (m, 2H), 4.70-4.00 (m, 6H), 3.98-3.82 (2H), 3.73-3.72 (m, 6H), 3.45-3.33 (m, 4H), 1.20-1.16 (m, 12H); 31P-NMR (162 MHz, DMSO-d6): δ 150.16, 149.92, 66.83, 66.74.

### Example 9: Preparation of Example 9 Dimer from 6R

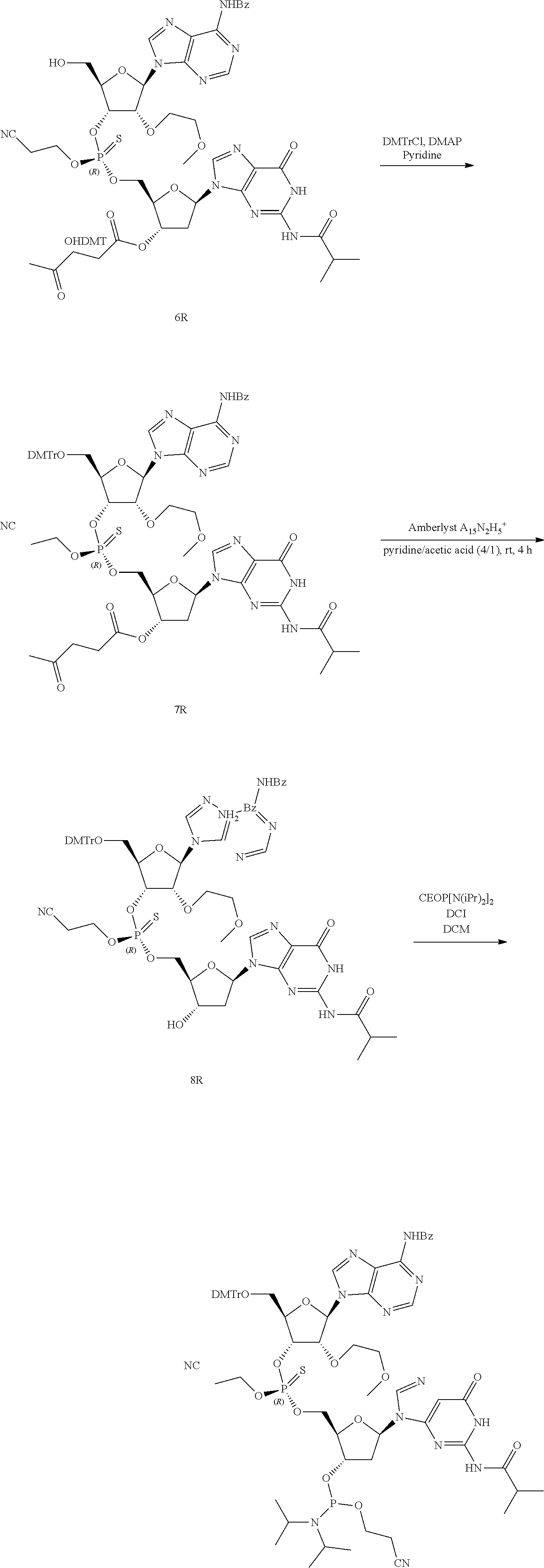

Preparation of (7R): To a stirred mixture of 6R (5.4 g, 5.4 mmol) in pyridine (55 mL) was added DMAP (134 mg, 1.1 mmol) and DMTrCl (3.6 g, 10.8 mmol) at r.t under N₂ atmosphere. The resulting mixture was stirred at r.t under argon atmosphere for 24 h. LCMS and TLC show SM was completely consumed. The reaction was quenched by the addition of sat. NaHCO₃ (aq.). The resulting mixture was extracted with EA. And the organic phase was washed with water and saturated brine and dried over by Na₂SO₄. Then the solution was concentrated under reduced pressure. The residue was purified by silica gel column chromatograph (eluent, DCM (0.5% pyridine) THF=1:0-0:1). This resulted in 7R (4.5 g, 49.6% yield) as a white solid. ESI-LCMS m/z1298.0 [M+H]⁺.

Preparation of (8R): To a solution of 7R (4.5 g, 3.5 mmol) in ACN (45 mL) was added N2H4 (0.5M, 17.5 ml) at 0° C. The reaction was stirred at 0° C. for 0.5 hour. LCMS showed 7S was consumed completely. The reaction mixture was quenched by 2,4-pentanedione at 0° C. for 15 minutes and extraction with EA. The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/1; Detector, UV 254 nm. This resulted in 8R (3.0 g, 72% yield) as a white solid. ESI-LCMS: m/z 1200.4 [M+H]+; 1H-NMR (400 MHZ, DMSO-d6): δ 11.31-11.12 (m, 2H), 8.73-8.52 (m, 3H), 8.04-8.03 (m, 4H), 7.99-7.26 (m, 15H), 7.26-7.25 (m, 1H), 6.91-6.87 (m, 4H), 6.62-6.59 (m, 1H), 6.01-5.79 (m, 2H), 5.68-5.56 (m, 1H), 5.21-5.02 (m, 1H), 4.70-4.00 (m, 6H), 3.73-3.72 (m, 6H), 3.50-3.45 (m, 4H), 2.90-2.80 (m, 2H); 31P-NMR (162 MHZ, DMSO-d6): δ 66.83, 66.58.

Preparation of Example 9 Dimer: To a solution of 8R (3.0 g, 2.5 mmol) in DCM (30 mL) with an inert atmosphere of nitrogen was added CEP[N(iPr)₂]₂ (979.6 mg, 3.2 mmol) and DCI (251 mg, 2.1 mmol) in order at room temperature. The resulting solution was stirred for 1.0 hour at room temperature and diluted with 30 mL dichloromethane. The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in Example 9 dimer (2.0 g, 56% yield) as yellow solid. ESI-LCMS: m/z 1368.2 [M−H]−; 1H-NMR (400 MHZ, DMSO-d6): δ11.31-11.12 (m, 2H), 8.70-8.58 (m, 3H), 8.05-8.00 (m, 4H), 7.98-7.26 (m, 15H), 7.26-7.25 (m, 1H), 6.91-6.88 (m, 4H), 6.62-6.59 (m, 1H), 6.01-5.76 (m, 2H), 5.24-5.50 (m, 2H), 4.70-4.00 (m, 6H), 3.98-3.82 (2H), 3.73-3.72 (m, 6H), 3.45-3.33 (m, 4H), 1.20-1.16 (m, 12H); 31P-NMR (162 MHz, DMSO-d6): δ 150.16, 149.92, 66.83, 66.74.

### Example 10: Preparation of Key Intermediate 6S & 6R for Example 11 and 12 Dimers

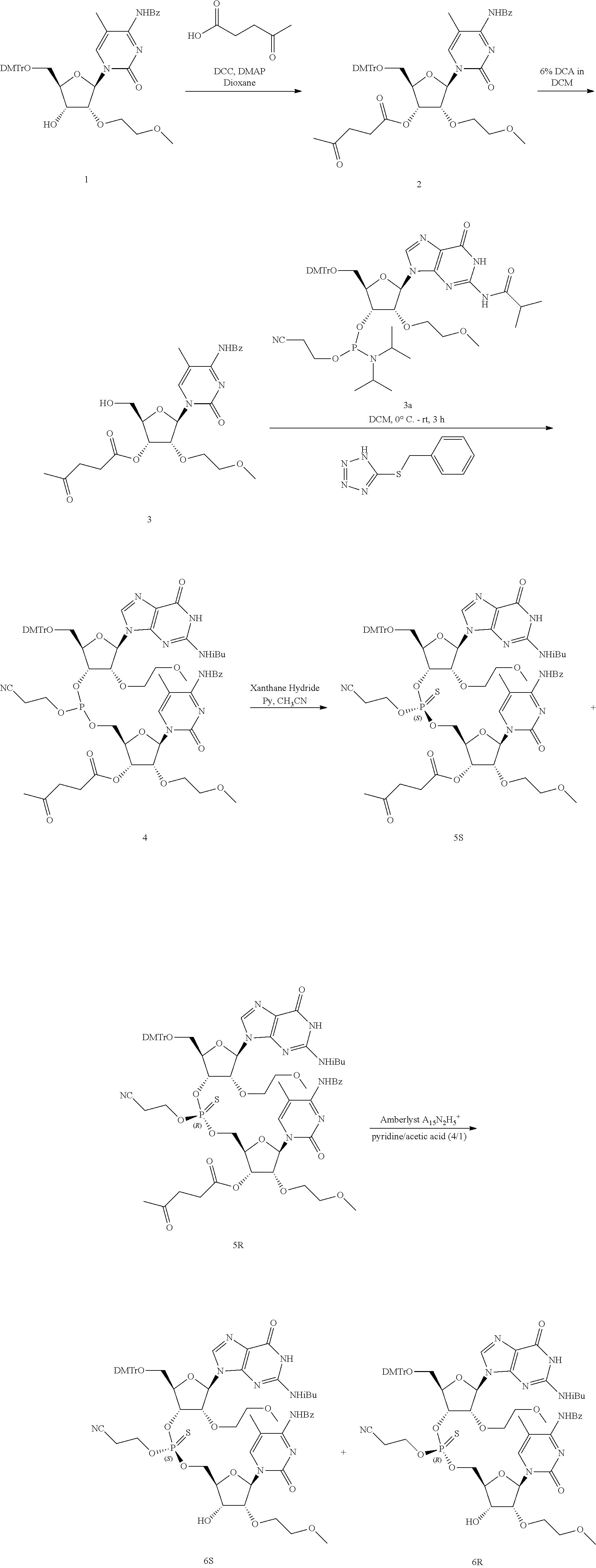

Preparation of (2): To a solution of 1 (17.0 g, 23.5 mmol) in dioxane (170 mL) was added DCC (6.6 g, 35.3 mmol) and DMAP (5.8 g, 28.2 mmol). Then the mixture was added Lev acid (4.1 g, 352.5 mmol) at 0° C. and the reaction mixture was stirred at room temperature for 1 hour. LCMS showed 1 was consumed completely. The reaction mixture was quenched by NaHCO₃ and extraction with DCM, washed with water and brine then dried over by anhydrous Na₂SO₄. Then the solution was concentrated under reduced pressure to give crude 2 (15.0 g) as solid which was used directly for the next step. ESI-LCMS: m/z 818.2 [M+H]+.

Preparation of (3): To a solution of 2 (15.0 g, 18.3 mmol) was dissolved 6% of DCA in DCM (150 mL) was added TES (3.2 g, 27.5 mmol) at r.t. The mixture was stirred at rt. for 10-20 min. LCMS and TLC show 2 was completely consumed. The reaction was quenched by the addition of sat. NaHCO₃ (aq.). The resulting mixture was extracted with DCM. Then the combined organic layers were washed the organic phase once with saturated brine and dried over by Na₂SO₄. Then the solution was concentrated under reduced pressure. This resulted was purified by slurry with EA. This resulted in 3 (8.0 g, 15.4 mmol, 84% yield) as a white solid. ESI-LCMS: m/z 518.2 [M+H]⁺.

Preparation of (4): To a solution of 3 (7.9 g, 15.3 mmol) and 3a (12.4 g, 13.6 mmol) in ACN (420 mL) was added molecular sieve and was stirred at room temperature for 15 min. Then the mixture was added BTT (0.3 M, 68 mL) and stirred at room temperature for 0.5 hour. LCMS showed 3 was consumed completely. The mixture is used directly.

Preparation of (5S&5R): The mixture of 4 was added pyridine and xanthane hydride (3.0 g, 20.4 mmol) then was stirred at room temperature for 15 minutes. After filtering, the mixture was extracted with EA, washed with water and brine then dried over by anhydrous Na₂SO₄. The mixture was concentrated to give the crude 5S&5R (15.0 g, 33.0 mmol, 97.8% purity, S/R=46:53 by PNMR). The mixture is used directly. ESI-LCMS: m/z 1360.2 [M−H]^{+ 31}P-NMR (400 MHz, DMSO-d6): δ 67.12, 66.80.

Preparation of (6S&6R): To a solution of 5S&5R (15.0 g, 10.9 mmol) in ACN (150 mL) was added N₂H₄. H₂O (0.5M, 54 mL) at 0° C. The reaction was stirred at 0° C. for 0.5 hours. LCMS showed 5S&5R was consumed completely. The reaction mixture was quenched by 2,4-pentanedione at 0° C. for 15 minutes and extraction with EA. Then washed the organic phase once with saturated brine and dried over by Na₂SO₄. Then the solution was concentrated under reduced pressure. The residue was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=1/0 within 25 min, the eluted product was collected at CH₃CN/H₂O (0.5% NH₄HCO₃)=1/1; Detector, UV 254 nm. This resulted in was purified by SFC. This resulted in 6R (3.0 g, 2.5 mmol,) and 6S (3.0 g, 2.5 mmol) as a white solid. ESI-LCMS: m/z 1264.1 [M+H]⁺; 6R: 1H NMR (400 MHZ, DMSO-d₆): δ 12.97 (s, 1H), 12.05 (s, 1H), 11.62 (s, 1H), 8.25-8.06 (m, 3H), 7.76-7.08 (m, 16H), 6.92-6.76 (m, 4H), 6.01-5.82 (m, 2H), 5.43-5.34 (m, 1H), 5.26-5.13 (m, 1H), 4.99-4.88 (m, 1H), 4.40-4.02 (m, 8H), 3.82-3.54 (m, 10H), 3.51-3.27 (m, 5H), 3.22 (s, 3H), 3.12 (s, 3H), 3.00-2.90 (m, 2H), 2.80-2.65 (m, 1H), 1.97 (s, 3H), 1.11 (dd, J=6.8, 1.3 Hz, 6H); ³¹P-NMR (400 MHZ, DMSO-d6): δ 67.09.

6S: ¹H NMR (400 MHZ, DMSO-d₆) δ 13.04 (s, 1H), 12.05 (s, 1H), 11.65 (s, 1H), 8.25-8.11 (m, 3H), 7.71-7.14 (m, 16H), 6.94-6.76 (m, 4H), 6.01-5.88 (m, 2H), 5.45-5.34 (m, 1H), 5.29-5.15 (m, 1H), 5.01-4.90 (m, 1H), 4.55-4.02 (m, 8H), 3.88-3.63 (m, 10H), 3.57-3.30 (m, 5H), 3.28 (s, 3H), 3.15 (s, 3H), 3.03-2.90 (m, 2H), 2.83-2.65 (m, 1H), 1.91 (s, 3H), 1.21-1.10 (m, 6H); ³¹P-NMR (400 MHZ, DMSO-d6): δ 66.81.

### Example 11: Preparation of Example 11 Dimer from Intermediate 6S

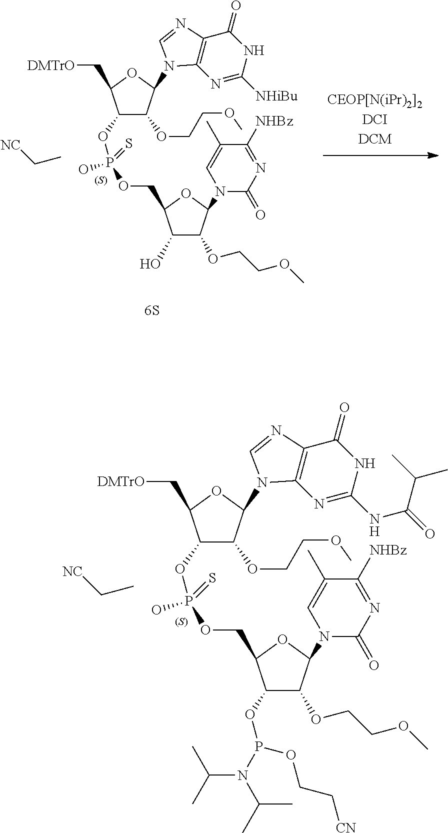

Preparation of Example 11 Dimer: To a solution of 6S (3.0 g, 2.4 mmol) in DCM (24 mL) with an inert atmosphere of nitrogen was added CEP[N(iPr)₂]₂ (928.7 mg, 3.1 mmol) and DCI (240 mg, 2.0 mmol) in order at room temperature. The resulting solution was stirred for 1.0 hour at room temperature and diluted with 50 mL dichloromethane and washed with 2×50 mL of saturated aqueous sodium bicarbonate and 1×50 mL of saturated aqueous sodium chloride respectively. The organic phase was dried over anhydrous sodium sulfate, filtered and concentrated till no residual solvent left under reduced pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=9/1; Detector, UV 254 nm. This resulted in Example 11 dimer (2.0 g, 1.3 mmol, 70.6% yield) as yellow solid. ESI-LCMS: m/z 1464.4 [M+H]⁺; ¹H NMR (400 MHZ, DMSO-d₆): δ 12.90 (s, 1H), 11.94 (s, 1H), 11.52 (d, J=3.2 Hz, 1H), 8.14-7.94 (m, 3H), 7.65-7.48 (m, 2H), 7.42 (t, J=7.6 Hz, 2H), 7.30 (d, J=7.3 Hz, 2H), 7.23-7.10 (m, 7H), 6.86-6.67 (m, 4H), 5.92-5.73 (m, 2H), 5.12 (dt, J=11.3, 5.3 Hz, 1H), 4.91-4.75 (m, 1H), 4.52-4.05 (m, 7H), 3.84-3.46 (m, 14H), 3.43-3.22 (m, 6H), 3.15 (d, J=7.0 Hz, 4H), 3.03 (s, 3H), 2.84 (qd, J=5.6, 2.6 Hz, 2H), 2.76-2.57 (m, 3H), 1.81 (s, 3H), 1.18-0.96 (m, 18H); ³¹P NMR (162 MHZ, DMSO-d₆): δ 149.62, 149.44, 66.86, 66.80.

### Example 12: Preparation of Example 12 Dimer from Intermediate 6R

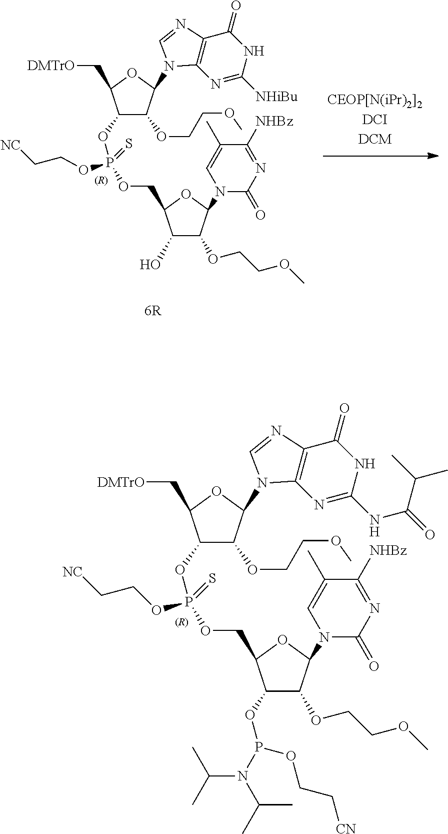

Preparation of Example 12 Dimer: To a solution of 6R (3.0 g, 2.4 mmol) in DCM (24 mL) with an inert atmosphere of nitrogen was added CEP[N(iPr)₂]₂ (928.7 mg, 3.1 mmol) and DCI (241 mg, 2.0 mmol) in order at room temperature. The resulting solution was stirred for 1.0 hour at room temperature and diluted with 50 mL dichloromethane and washed with 2×50 mL of saturated aqueous sodium bicarbonate and 1×50 mL of saturated aqueous sodium chloride respectively. The organic phase was dried over anhydrous sodium sulfate, filtered and concentrated till no residual solvent left under reduced pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH3CN/H₂O (0.5% NH₄HCO₃)=1/0 within 20 min, the eluted product was collected at CH₃CN/H₂O (0.5% NH₄HCO₃)=9/1; Detector, UV 254 nm. This resulted in Example 12 dimer (2.0 g, 1.3 mmol, 70.6% yield) as yellow solid. ESI-LCMS: m/z 1365.3 [M+H]⁺; ¹H NMR (400 MHZ, DMSO-d₆): δ 12.88 (s, 1H), 11.97 (s, 1H), 11.52 (s, 1H), 8.06 (dd, J=26.3, 6.1 Hz, 3H), 7.63 (d, J=9.2 Hz, 1H), 7.52 (t, J=7.4 Hz, 1H), 7.42 (t, J=7.6 Hz, 2H), 7.33-7.25 (m, 2H), 7.24-7.09 (m, 7H), 6.76 (td, J=5.8, 2.7 Hz, 4H), 5.88-5.70 (m, 2H), 5.22-5.05 (m, 1H), 4.86 (t, J=6.2 Hz, 1H), 4.41-4.03 (m, 5H), 3.90-3.44 (m, 14H), 3.43-3.23 (m, 5H), 3.14 (d, J=6.8 Hz, 4H), 3.05 (d, J=1.6 Hz, 3H), 2.88 (td, J=5.9, 2.4 Hz, 2H), 2.68 (dq, J=15.6, 6.4 Hz, 3H), 1.95-1.82 (m, 3H), 1.24-0.96 (m, 18H); ³¹P NMR (162 MHZ, DMSO-d₆): δ 149.56, 149.34, 67.43, 67.33.

### Example 13: Preparation of Key Intermediate 6S & 6R for Example 14 and 15 Dimers

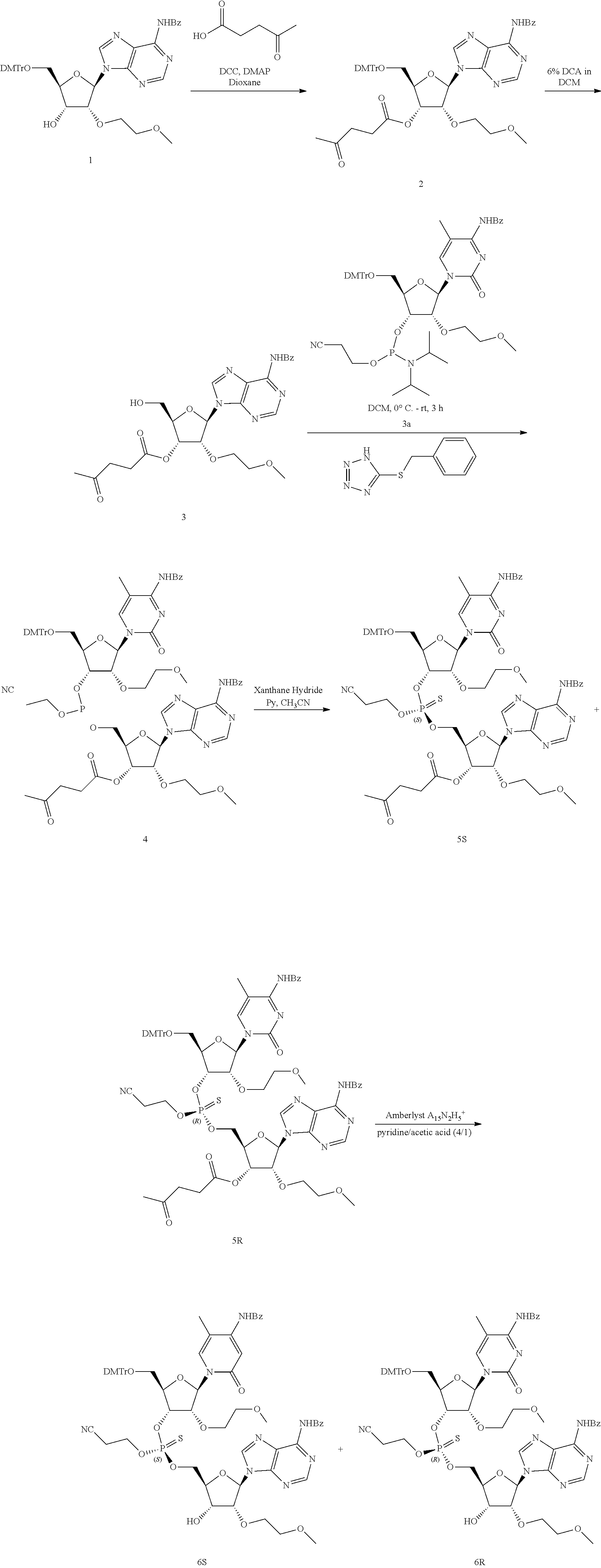

Preparation of (2): To a solution of 1 (32.0 g, 22.6 mmol) in dioxane (320 mL) was added DCC (13.2 g, 64.0 mmol) and DMAP (6.3 g, 51.8 mmol). Then added Lev acid (7.2 g, 621.0 mmol) at 0° C. and the reaction mixture was stirred at room temperature for 1 hour. LCMS showed 1 was consumed completely. The reaction mixture was quenched by NaHCO₃ and extraction with DCM, washed with water and brine then dried over by anhydrous Na₂SO₄. Then the solution was concentrated under reduced pressure to give crude 2 (30.0 g) as solid which was used directly for the next step. ESI-LCMS: m/z 828.2 [M−H]⁻.

Preparation of (3): To a solution of 2 (29.5 g, 35.1 mmol) was dissolved 6% of DCA in DCM (300 mL) was added TES (6.2 g, 32.5 mmol) at r.t. The mixture was stirred at rt. for 10-20 min. LCMS and TLC show 2 was completely consumed. The reaction was quenched by the addition of NaHCO₃ (aq.). The resulting mixture was extracted with DCM. And the organic phase was washed with water and saturated brine and dried over by Na₂SO₄. Then the solution was concentrated under reduced pressure. The residue was purified by silica gel column chromatograph (eluent, PE/EA=3:1˜1:2). This resulted in giving 3 (16.0 g, 30.0 mmol, 85.0% yield) as a white solid. ESI-LCMS: m/z 562.1 [M−H]⁻; ¹H NMR (400 MHZ, DMSO-d₆) δ 11.25 (s, 1H), 8.77 (d, J=9.6 Hz, 2H), 8.09-8.01 (m, 2H), 7.70-7.61 (m, 1H), 7.56 (dd, J=8.2, 6.8 Hz, 2H), 6.14 (d, J=6.9 Hz, 1H), 5.47 (dd, J=5.1, 2.4 Hz, 1H), 5.35 (t, J=5.7 Hz, 1H), 4.94 (dd, J=7.0, 5.2 Hz, 1H), 4.19 (td, J=3.9, 2.3 Hz, 1H), 3.76-3.58 (m, 2H), 3.54 (td, J=4.5, 1.3 Hz, 2H), 3.32-3.22 (m, 2H), 3.03 (s, 3H), 2.79 (dd, J=7.2, 5.7 Hz, 2H), 2.61 (ddd, J=8.1, 5.8, 1.7 Hz, 2H), 2.15 (s, 3H).

Preparation of (4): To a solution of 3 (10.0 g, 18.9 mmol) and 3a (18.3 g, 20.0 mmol) in ACN (180 mL) was added molecular sieve and was stirred at room temperature for 15 min. Then the mixture was added BTT (0.3 M, 94 mL) and stirred at room temperature for 0.5 hour. LCMS showed 3 was consumed completely. The mixture used for directly.

Preparation of (5S&5R): The mixture 4 was added pyridine and xanthane hydride (5.7 g, 37.9 mmol) then was stirred at room temperature for 15 minutes. After filtering, the mixture was extracted with EA, washed with water and brine then dried over by anhydrous Na₂SO₄. The mixture was concentrated to give the crude 5S&5R (17.5 g, 12.8 mmol, S/R=44:55 by PNMR). The mixture is used directly. ESI-LCMS: m/z 1378.2 [M−H]^{+ 31}P-NMR (400 MHZ, DMSO-d6): δ 67.01, 66.80.

Preparation of (6S&6R): To a solution of 5 (17.0 g, 12.3 mmol) in ACN (170 mL) was added N₂H₄ (0.5M, 62 mL) at 0° C. The reaction was stirred at 0° C. for 0.5 hours. LCMS showed 5S&5R was consumed completely. The reaction mixture was quenched by 2,4-pentanedione at 0° C. for 15 minutes and extraction with EA. Then washed the organic phase once with saturated brine and dried over by Na₂SO₄. Then the solution was concentrated under reduced pressure. The residue was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=1/0 within 25 min, the eluted product was collected at CH₃CN/H₂O (0.5% NH₄HCO₃)=3/1; Detector, UV 254 nm. The residue was purified by SFC. This resulted in 6R (3.1 g, 2.4 mmol, 97.8% purity), and 6S (3.2 g, 2.4 mmol, 97.8% purity) as a white solid. 6R: ESI-LCMS: m/z 1282.3 [M+H]+; ¹H NMR (400 MHZ, DMSO-d₆) δ 12.86 (s, 1H), 8.25-7.95 (m, 4H), 7.76 (s, 1H), 7.68-7.46 (m, 6H), 7.46-7.38 (m, 2H), 7.36-7.25 (m, 7H), 6.95-6.84 (m, 4H), 6.17 (d, J=5.2 Hz, 1H), 5.91 (d, J=5.3 Hz, 1H), 5.46 (s, 1H), 5.20 (dt, J=10.0, 4.7 Hz, 1H), 4.67 (t, J=5.2 Hz, 1H), 4.45 (dd, J=10.1, 5.4 Hz, 2H), 4.38-4.10 (m, 6H), 3.93-3.57 (m, 11H), 3.45 (dt, J=25.0, 4.3 Hz, 5H), 3.19 (s, 3H), 3.14 (s, 3H), 1.57 (s, 3H). ³¹P NMR: δ 66.96.

6S: ESI-LCMS: m/z 1282.3 [M+H]+; ¹H NMR (400 MHZ, DMSO-d₆) δ 12.83 (s, 1H), 8.76 (s, 1H), 8.65 (s, 1H), 8.16 (d, J=7.5 Hz, 2H), 8.10-8.00 (m, 2H), 7.74 (s, 1H), 7.67-7.46 (m, 6H), 7.41 (dt, J=6.8, 1.3 Hz, 2H), 7.34-7.24 (m, 7H), 7.03-6.79 (m, 4H), 6.18 (d, J=5.3 Hz, 1H), 5.93 (d, J=5.8 Hz, 1H), 5.47 (d, J=5.8 Hz, 1H), 5.21 (dt, J=9.6, 4.4 Hz, 1H), 4.69 (t, J=5.2 Hz, 1H), 4.54-4.38 (m, 3H), 4.38-4.05 (m, 5H), 3.84-3.60 (m, 10H), 3.40 (dt, J=18.3, 4.6 Hz, 5H), 1.59 (s, 3H). ³¹P NMR: δ 66.80.

### Example 14: Preparation of Example 14 Dimer from Intermediate 6S & 6R

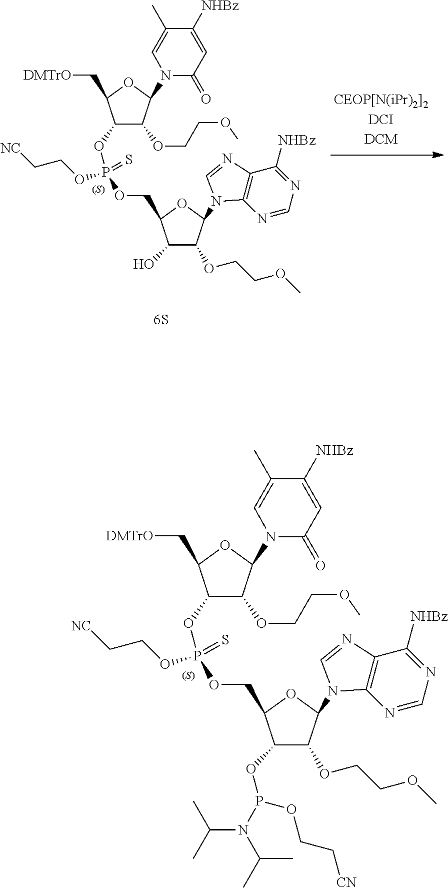

Preparation of Example 14 Dimer: To a solution of 6S (3.0 g, 2.3 mmol) in DCM (30 mL) was added DCI (235 mg, 1.9 mmol) and CEP[N(iPr)₂]₂ (845 mg, 2.8 mmol) under N₂. The mixture was stirred at 25° C. for 2.5 h. LCMS showed 6S was consumed completely. The product was extracted with DCM. The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in Example 14 dimer (2.0 g, 1.3 mmol, 56% yield) as a white solid. ESI-LCMS: m/z 1483.3 [M+2H]⁺; ¹H NMR (600 MHZ, DMSO-d₆) δ 12.78 (s, 1H), 11.18 (s, 1H), 8.56 (d, J=2.4 Hz, 2H), 8.17-7.91 (m, 4H), 7.71-7.37 (m, 7H), 7.38-7.10 (m, 9H), 6.81 (dd, J=8.8, 3.2 Hz, 4H), 6.09 (dd, J=13.6, 5.4 Hz, 1H), 5.96-5.69 (m, 1H), 5.13 (dq, J=10.7, 5.5, 4.4 Hz, 1H), 4.90-4.61 (m, 2H), 4.51-4.07 (m, 7H), 3.88-3.50 (m, 14H), 3.46-3.28 (m, 4H), 3.20 (dd, J=12.4, 9.6 Hz, 1H), 3.15-2.97 (m, 6H), 2.93-2.63 (m, 4H), 1.49 (d, J=4.6 Hz, 3H), 1.11 (dd, J=13.1, 6.7 Hz, 12H). ³¹P NMR (400 MHZ, DMSO-d₆): δ 149.69, 149.29, 67.06, 66.99.

### Example 15: Preparation of Example 15 Dimer from Intermediate 6R

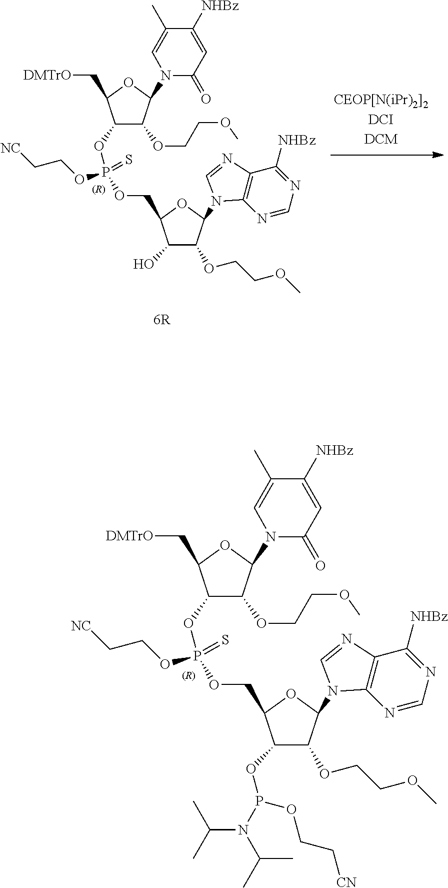

Preparation of Example 15 Dimer: To a solution of 6R (3.0 g, 2.3 mmol) in DCM (30 mL) was added DCI (235 mg, 1.9 mmol) and CEP[N(iPr)₂]₂ (845 mg, 2.8 mmol) under N₂. The mixture was stirred at 25° C. for 2.5 h. LCMS showed 6R was consumed completely. The product was extracted with DCM. The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in Example 15 dimer (2.0 g, 1.3 mmol, 56% yield) as a white solid. ESI-LCMS: m/z 1482.3 [M+H]⁺; ¹H NMR (400 MHZ, DMSO-d₆) δ 12.74 (s, 1H), 11.15 (s, 1H), 8.89-8.44 (m, 2H), 8.14-7.89 (m, 4H), 7.70-7.37 (m, 7H), 7.37-7.11 (m, 9H), 6.11 (dd, J=13.5, 5.5 Hz, 1H), 5.86 (d, J=6.6 Hz, 1H), 5.12 (s, 1H), 4.93-4.58 (m, 2H), 4.53-4.16 (m, 5H), 3.94-3.46 (m, 14H), 3.38-3.26 (m, 5H), 3.21 (d, J=2.7 Hz, 1H), 3.10-2.94 (m, 6H), 2.86-2.66 (m, 4H), 1.50 (s, 3H), 1.29-0.97 (m, 12H). ³¹P NMR (400 MHZ, DMSO-d₆): δ 149.87, 149.24, 66.95, 66.90.

### Example 16: Preparation of Key Intermediate 6S & 6R for Example 17 and 18 Dimers

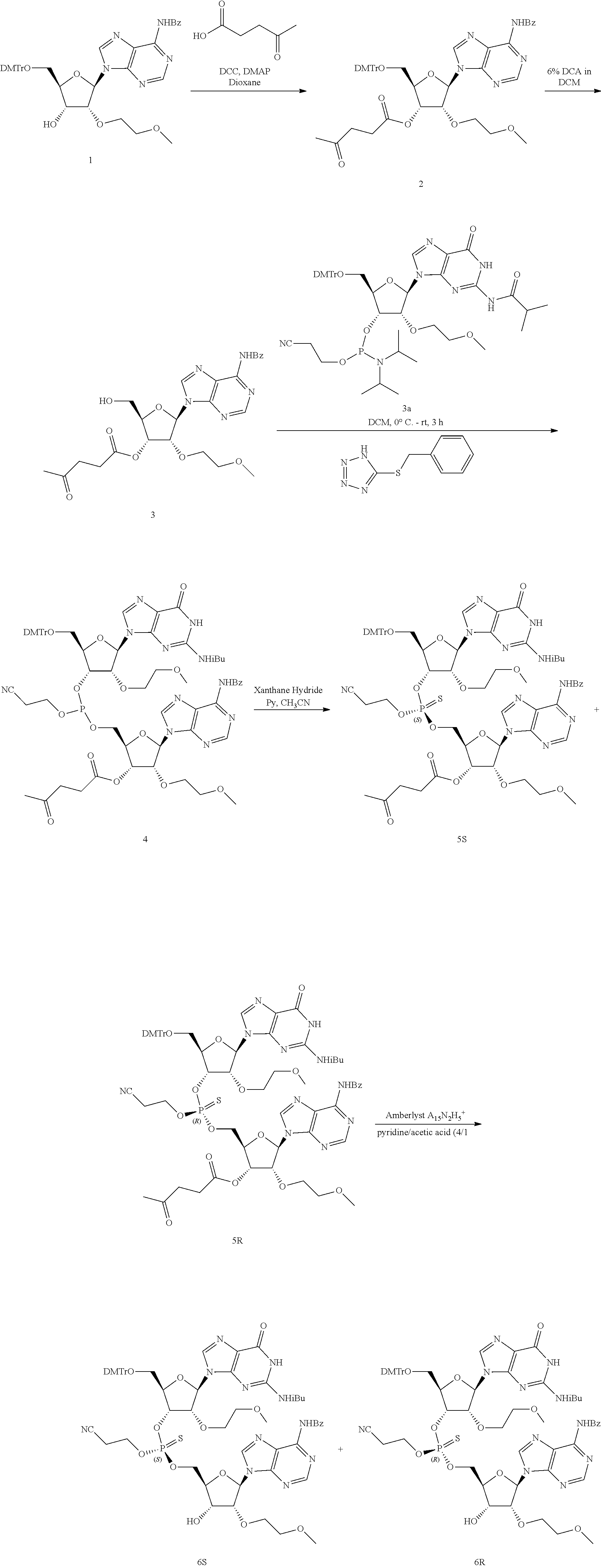

Preparation of (2): To a solution of 1 (16.0 g, 21.3 mmol) in dioxane (160 mL) was added DCC (6.6 g, 32.0 mmol) and DMAP (3.2 g, 25.9 mmol). Then added Lev acid (3.6 g, 310.8 mmol) at 0° C. and the reaction mixture was stirred at room temperature for 1 hour. LCMS showed 1 was consumed completely. The reaction mixture was quenched by NaHCO₃ and extraction with DCM, washed with water and brine then dried over by anhydrous Na₂SO₄. Then the solution was concentrated under reduced pressure to give crude 2 (15.0 g) as solid which was used directly for the next step. ESI-LCMS: m/z 828.2 [M−H]⁻.

Preparation of (3): To a solution of 2 (14.7 g, 17.6 mmol) was dissolved 6% of DCA in DCM (150 mL) was added TES (3.1 g, 26.4 mmol) at r.t. The mixture was stirred at rt. for 10-20 min. LCMS and TLC show 2 was completely consumed. The reaction was quenched by the addition of NaHCO₃ (aq.). The resulting mixture was extracted with DCM. And the organic phase was washed with water and saturated brine and dried over by Na₂SO₄. Then the solution was concentrated under reduced pressure. The residue was purified by silica gel column chromatograph (eluent, PE/EA=3:1˜ 1:2). This resulted in giving 3 (8.0 g, 15.0 mmol, 85.0% yield) as a white solid. ESI-LCMS: m/z 562.1 [M−H]⁻; ¹H NMR (400 MHZ, DMSO-d₆) δ 11.25 (s, 1H), 8.77 (d, J=9.6 Hz, 2H), 8.09-8.01 (m, 2H), 7.70-7.61 (m, 1H), 7.56 (dd, J=8.2, 6.8 Hz, 2H), 6.14 (d, J=6.9 Hz, 1H), 5.47 (dd, J=5.1, 2.4 Hz, 1H), 5.35 (t, J=5.7 Hz, 1H), 4.94 (dd, J=7.0, 5.2 Hz, 1H), 4.19 (td, J=3.9, 2.3 Hz, 1H), 3.76-3.58 (m, 2H), 3.54 (td, J=4.5, 1.3 Hz, 2H), 3.32-3.22 (m, 2H), 3.03 (s, 3H), 2.79 (dd, J=7.2, 5.7 Hz, 2H), 2.61 (ddd, J=8.1, 5.8, 1.7 Hz, 2H), 2.15 (s, 3H).

Preparation of (4): To a solution of 3 (7.9 g, 15.0 mmol) and 3a (12.4 g, 13.6 mmol) in ACN (420 mL) was added molecular sieve and was stirred at room temperature for 15 min. Then the mixture was added BTT (0.3 M, 68 mL) and stirred at room temperature for 0.5 hour. LCMS showed 3 was consumed completely. The mixture is used directly.

Preparation of (5R&5S): The mixture 4 was added pyridine and xanthane hydride (3.0 g, 20.4 mmol) then was stirred at room temperature for 15 minutes. After filtering, the mixture was extracted with EA, washed with water and brine then dried over by anhydrous Na₂SO₄. The mixture was concentrated to give the crude 5S&5R (15.0 g, 11.1 mmol, S/R=49:50 by PNMR). The mixture is used directly. ESI-LCMS: m/z 1355.2 [M−H]^{+ 31}P-NMR (400 MHZ, DMSO-d6): δ 67.12, 66.80.

Preparation of (6): To a solution of 5 (15.0 g, 10.9 mmol) in ACN (150 mL) was added N₂H₄ (0.5M, 55 mL) at 0° C. The reaction was stirred at 0° C. for 0.5 hours. LCMS showed 5S&5R was consumed completely. The reaction mixture was quenched by 2,4-pentanedione at 0° C. for 15 minutes and extraction with EA. Then washed the organic phase once with saturated brine and dried over by Na₂SO₄. Then the solution was concentrated under reduced pressure. The residue was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=1/0 within 25 min, the eluted product was collected at CH₃CN/H₂O (0.5% NH₄HCO₃)=2/1; Detector, UV 254 nm. The residue was purified by SFC. This resulted in 6R (3.0 g, 2.3 mmol,), and 6S (3.0 g, 2.3 mmol,) as a white solid. 6R: ESI-LCMS: m/z 1274.4 [M+H]⁺. ¹H NMR (400 MHZ, DMSO-d₆) δ:12.07 (s, 1H), 11.62 (s, 1H), 11.22 (s, 1H), 8.76 (s, 1H), 8.65 (s, 1H), 8.21-7.96 (m, 3H), 7.71-7.49 (m, 3H), 7.41-7.09 (m, 11H), 6.92-6.78 (m, 4H), 6.19 (d, J=5.1 Hz, 1H), 5.90 (d, J=7.6 Hz, 1H), 5.48 (dd, J=5.8, 1.6 Hz, 1H), 5.27-5.11 (m, 1H), 4.96-4.84 (m, 1H), 4.73-4.64 (m, 1H), 4.53-4.13 (m, 7H), 3.82-3.55 (m, 10H), 3.46-3.55 (m, 4H), 3.14 (s, 3H), 3.09 (s, 3H), 2.94-2.89 (m, 2H), 2.78-2.64 (m, 1H), 1.11 (d, J=6.8 Hz, 6H); ³¹P NMR (400 MHZ, DMSO-d₆): δ 67.22.

6S: ESI-LCMS: m/z 1274.4 [M+H]⁺. ¹H NMR (400 MHZ, DMSO-d₆) δ:12.05 (s, 1H), 11.58 (s, 1H), 11.19 (s, 1H), 8.73 (s, 1H), 8.63 (s, 1H), 8.14-6.97 (m, 3H), 7.73-7.49 (m, 3H), 7.49-7.07 (m, 11H), 6.95-6.78 (m, 4H), 6.18 (d, J=5.1 Hz, 1H), 5.90 (d, J=7.6 Hz, 1H), 5.47 (dd, J=5.8, 1.6 Hz, 1H), 5.24-5.09 (m, 1H), 4.96-4.82 (m, 1H), 4.73-4.63 (m, 1H), 4.57-4.10 (m, 7H), 3.83-3.50 (m, 10H), 3.46-3.40 (m, 2H), 3.29-3.23 (m, 2H), 3.14 (s, 3H), 3.00 (s, 3H), 2.94-2.86 (m, 2H), 2.78-2.64 (m, 1H), 1.10 (d, J=6.8 Hz, 6H); ³¹P NMR (162 MHz, DMSO-d₆): δ 67.10.

### Example 17: Preparation of Example 17 Dimer from Intermediate 6S

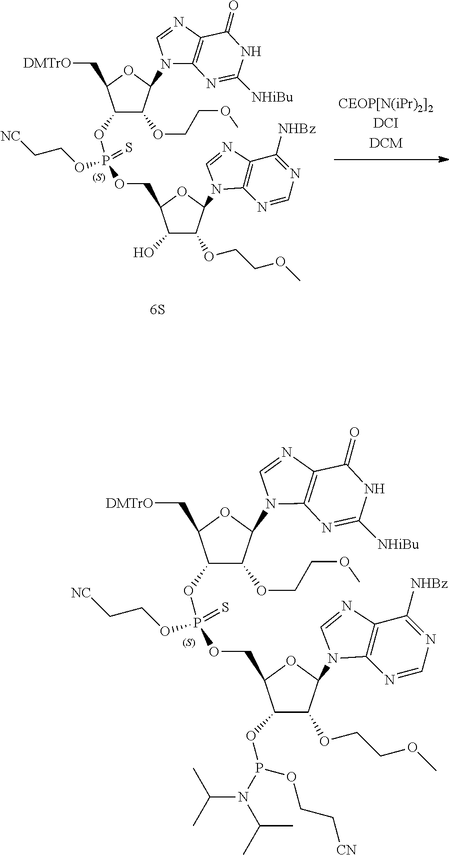

Preparation of Example 17 Dimer: To a solution of 6S (3.0 g, 2.4 mmol) in DCM (24 mL) with an inert atmosphere of nitrogen was added CEP[N(IPr)₂]₂ (939 mg, 3.1 mmol) and DCI (241 mg, 2.1 mmol) in order at room temperature. The resulting solution was stirred for 1 hour at room temperature and diluted with 50 mL dichloromethane and washed with 2×50 mL of saturated aqueous sodium bicarbonate and 1×50 mL of saturated aqueous sodium chloride respectively. The organic phase was dried over anhydrous sodium sulfate, filtered and concentrated till no residual solvent left under reduced pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=9/1; Detector, UV 254 nm. This resulted in Example 17 dimer (2.0 g, 1.3 mmol, 56.5% yield) as white solid. ESI-LCMS: m/z 1474.3 [M+H]⁺; ¹H-NMR (400 MHZ, DMSO-d₆): δ 12.06 (s, 1H), 11.59 (s, 1H), 11.21 (s, 1H), 8.83-8.58 (m, 2H), 8.15-7.96 (m, 3H), 7.73-7.61 (m, 1H), 7.55 (t, J=7.6 Hz, 2H), 7.42-7.12 (m, 9H), 6.92-6.81 (m, 4H), 6.20 (dd, J=13.1, 5.1 Hz, 1H), 5.88 (dd, J=7.8, 1.9 Hz, 1H), 5.16 (dt, J=10.3, 4.7 Hz, 1H), 4.88 (dt, J=10.0, 5.2 Hz, 2H), 4.84-4.66 (m, 1H), 4.50 (dd, J=18.6, 7.8 Hz, 4H), 3.95-3.49 (m, 14H), 3.40 (dt, J=9.3, 4.6 Hz, 2H), 3.33 (s, 2H), 3.28-3.15 (m, 3H), 3.12 (d, J=3.3 Hz, 3H), 3.00 (d, J=4.4 Hz, 3H), 2.89 (td, J=7.1, 5.3 Hz, 2H), 2.84-2.78 (m, 2H), 2.72 (p, J=6.9 Hz, 1H), 1.19 (dt, J=10.7, 6.6 Hz, 12H), 1.10 (d, J=6.8 Hz, 6H); ³¹P NMR (400 MHZ, DMSO-d₆): δ 149.74, 149.33, 67.19.

### Example 18: Preparation of Example 18 Dimer from Intermediate 6R

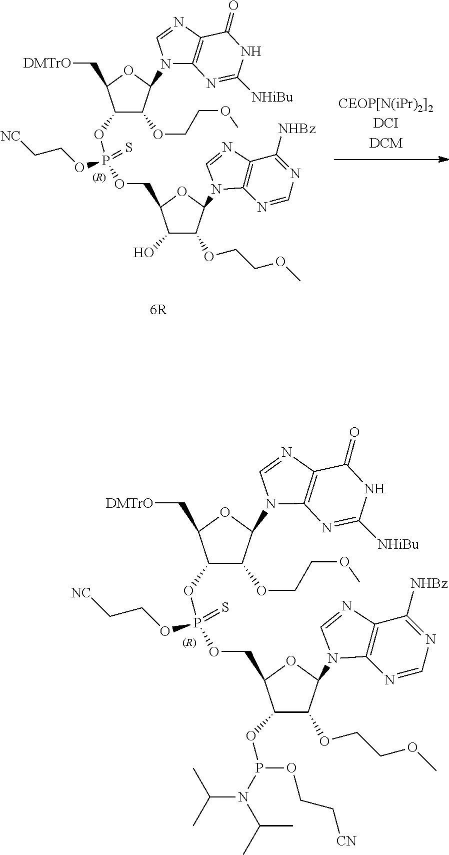

Preparation of Example 18 Dimer: To a solution of 6R (3.0 g, 2.4 mmol) in DCM (24 mL) with an inert atmosphere of nitrogen was added CEP[N(iPr)₂]₂ (939 mg, 3.1 mmol) and DCI (241 mg, 2.1 mmol) in order at room temperature. The resulting solution was stirred for 1 hour at room temperature and diluted with 50 mL dichloromethane and washed with 2×50 mL of saturated aqueous sodium bicarbonate and 1×50 mL of saturated aqueous sodium chloride respectively. The organic phase was dried over anhydrous sodium sulfate, filtered and concentrated till no residual solvent left under reduced pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=9/1; Detector, UV 254 nm. This resulted in Example 18 dimer (2.0 g, 1.3 mmol, 56.5% yield) as white solid. ESI-LCMS: m/z 1474.3 [M+H]⁺; ¹H NMR (400 MHZ, DMSO-d₆) δ 12.07 (s, 1H), 11.60 (s, 1H), 11.24 (s, 1H), 8.76 (s, 1H), 8.68 (d, J=1.9 Hz, 1H), 8.27-7.94 (m, 3H), 7.70-7.61 (m, 1H), 7.55 (t, J=7.6 Hz, 2H), 7.41-7.15 (m, 9H), 6.84 (ddt, J=9.5, 5.3, 2.1 Hz, 4H), 6.21 (dd, J=13.5, 5.3 Hz, 1H), 5.90 (dd, J=7.7, 1.7 Hz, 1H), 5.28-5.03 (m, 1H), 4.95-4.87 (m, 2H), 4.78 (dq, J=9.7, 5.1, 4.4 Hz, 1H), 4.48-4.09 (m, 6H), 3.99-3.52 (m, 14H), 3.47-3.29 (m, 6H), 3.22 (dt, J=10.0, 4.3 Hz, 1H), 3.18-3.02 (m, 6H), 2.89 (td, J=5.9, 3.0 Hz, 2H), 2.82-2.62 (m, 2H), 1.26-1.14 (m, 12H), 1.13-1.07 (m, 6H); ³¹P NMR (400 MHZ, DMSO-d₆): δ 149.55, 149.35, 67.40, 67.33.

### Example 19: Preparation of Key Intermediate 6S & 6R for Example 20 and 21 Dimers

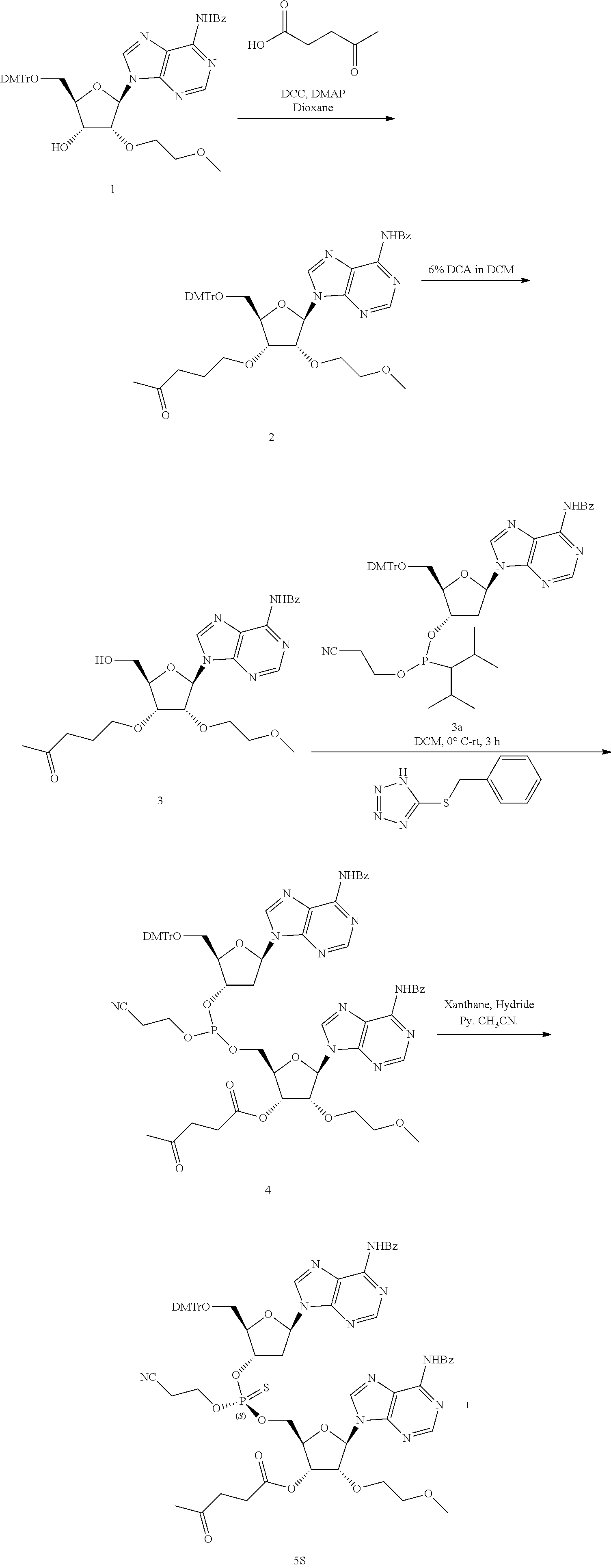

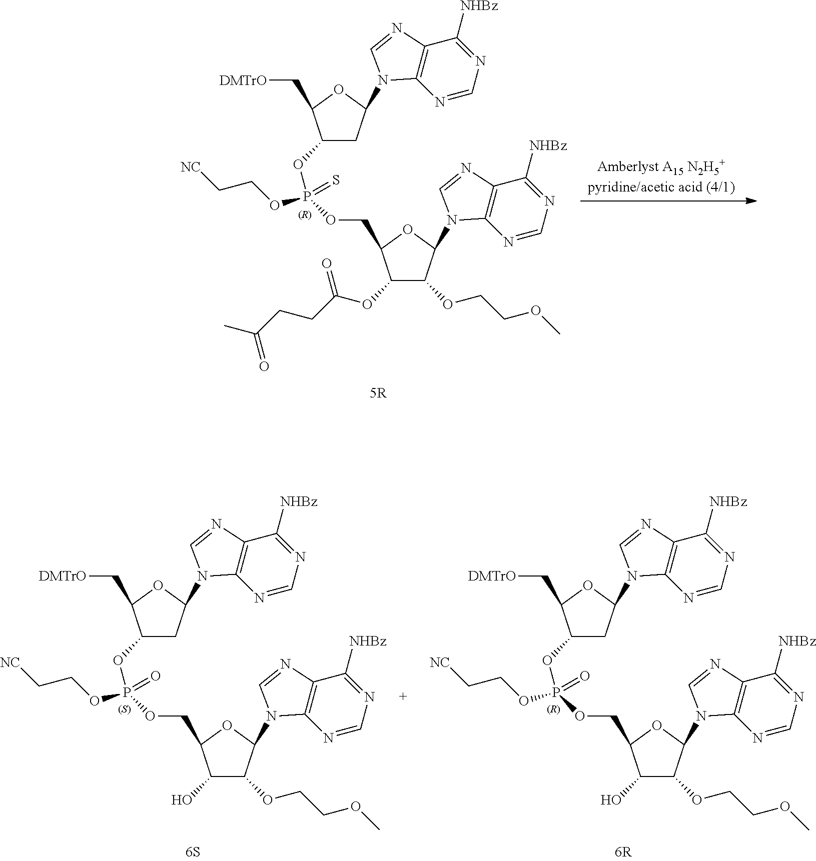

Preparation of (2): To a solution of 1 (32.0 g, 22.6 mmol) in dioxane (320 mL) was added DCC (13.2 g, 64.0 mmol) and DMAP (6.3 g, 51.8 mmol). Then added Lev acid (7.2 g, 621.0 mmol) at 0° C. and the reaction mixture was stirred at room temperature for 1 hour. LCMS showed 1 was consumed completely. The reaction mixture was quenched by NaHCO₃ and extraction with DCM, washed with water and brine then dried over by anhydrous Na₂SO₄. Then the solution was concentrated under reduced pressure to give crude 2 (30.0 g) as solid which was used directly for the next step. ESI-LCMS: m/z 828.2 [M−H]⁻.

Preparation of (3): To a solution of 2 (29.5 g, 35.1 mmol) was dissolved 6% of DCA in DCM (300 mL) was added TES (6.2 g, 32.5 mmol) at r.t. The mixture was stirred at rt. for 10-20 min. LCMS and TLC show 2 was completely consumed. The reaction was quenched by the addition of NaHCO₃ (aq.). The resulting mixture was extracted with DCM. And the organic phase was washed with water and saturated brine and dried over by Na₂SO₄. Then the solution was concentrated under reduced pressure. The residue was purified by silica gel column chromatograph (eluent, PE/EA=3:1˜1:2). This resulted in giving 3 (16.0 g, 30.0 mmol, 85.0% yield) as a white solid. ESI-LCMS: m/z 562.1 [M−H]⁻; ¹H NMR (400 MHZ, DMSO-d₆) δ 11.25 (s, 1H), 8.77 (d, J=9.6 Hz, 2H), 8.09-8.01 (m, 2H), 7.70-7.61 (m, 1H), 7.56 (dd, J=8.2, 6.8 Hz, 2H), 6.14 (d, J=6.9 Hz, 1H), 5.47 (dd, J=5.1, 2.4 Hz, 1H), 5.35 (t, J=5.7 Hz, 1H), 4.94 (dd, J=7.0, 5.2 Hz, 1H), 4.19 (td, J=3.9, 2.3 Hz, 1H), 3.76-3.58 (m, 2H), 3.54 (td, J=4.5, 1.3 Hz, 2H), 3.32-3.22 (m, 2H), 3.03 (s, 3H), 2.79 (dd, J=7.2, 5.7 Hz, 2H), 2.61 (ddd, J=8.1, 5.8, 1.7 Hz, 2H), 2.15 (s, 3H).

Preparation of (4): To a solution of 3 (10.0 g, 18.9 mmol) and 3a (17.1 g, 20.0 mmol) in ACN (180 mL) was added molecular sieve and was stirred at room temperature for 15 min. Then the mixture was added BTT (0.3 M, 94 mL) and stirred at room temperature for 0.5 hour. LCMS showed 3 was consumed completely. The mixture used for directly.

Preparation of (5S&5R): The mixture 4 was added pyridine and xanthane hydride (5.7 g, 37.9 mmol) then was stirred at room temperature for 15 minutes. After filter, the mixture was extracted with EA, washed with water and brine then dried over by anhydrous Na₂SO₄. The mixture was concentrated to give the mixture 5S&5R (16.0 g, 12.1 mmol, S/R=40:55 by SFC). The mixture used for directly. ESI-LCMS: m/z 1315.2 [M−H]^{+ 31}P-NMR (400 MHZ, DMSO-d6): δ 66.44.

Preparation of (6): To a solution of 5 (16.0 g, 12.1 mmol) in ACN (160 mL) was added N₂H₄ (0.5M, 62 mL) at 0° C. The reaction was stirred at 0° C. for 0.5 hour. LCMS showed 5S&5R was consumed completely. The reaction mixture was quenched by 2,4-pentanedione at 0° C. for 15 minutes and extraction with EA. Then washed the organic phase once with saturated brine and dried over by Na₂SO₄. Then the solution was concentrated under reduced pressure. The residue was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=1/0 within 25 min, the eluted product was collected at CH₃CN/H₂O (0.5% NH₄HCO₃)=3/1; Detector, UV 254 nm. The residue was purified by SFC. This resulted in 6R (3.0 g, 2.4 mmol), and 6S (3.2 g, 2.5 mmol,) as a white solid. 6R: ESI-LCMS: m/z 12182.3 [M+H]+; ¹H NMR (400 MHz, DMSO-d₆) δ 11.20 (s, 2H), 8.81-8.44 (m, 4H), 8.16-7.92 (m, 4H), 7.64 (tt, J=6.6, 3.3 Hz, 2H), 7.55 (dd, J=7.8, 6.4 Hz, 4H), 7.42-7.33 (m, 2H), 7.30-7.19 (m, 9H), 6.88-6.75 (m, 4H), 6.48 (t, J=7.0 Hz, 1H), 6.17 (d, J=5.1 Hz, 1H), 5.35 (dd, J=9.1, 5.6 Hz, 2H), 4.70 (t, J=5.1 Hz, 1H), 4.56-4.26 (m, 4H), 4.19 (ddt, J=19.0, 10.2, 4.6 Hz, 3H), 3.83-3.58 (m, 8H), 3.50-3.16 (m, 6H), 2.89 (t, J=5.9 Hz, 2H), 2.64 (ddd, J=14.3, 6.5, 2.4 Hz, 1H). ³¹P NMR: (400 MHZ, DMSO-d₆): δ 66.41.

6S: ESI-LCMS: m/z 1218.2 [M+H]+; ¹H NMR (400 MHZ, DMSO-d₆) δ 11.22 (s, 2H), 8.90-8.47 (m, 4H), 8.13-7.95 (m, 4H), 7.73-7.49 (m, 6H), 7.44-7.31 (m, 2H), 7.30-7.20 (m, 9H), 6.88-6.75 (m, 4H), 6.51 (t, J=7.0 Hz, 1H), 6.21 (d, J=5.2 Hz, 1H), 5.45 (d, J=33.2 Hz, 2H), 4.70 (t, J=5.1 Hz, 1H), 4.48 (s, 1H), 4.21 (dt, J=9.2, 5.7 Hz, 3H), 3.83-3.57 (m, 8H), 3.48-3.19 (m, 6H), 2.90 (t, J=5.9 Hz, 2H), 2.80-2.62 (m, 1H). ³¹P NMR: (400 MHZ, DMSO-d₆): δ 66.44.

### Example 20: Preparation of Example 20 Dimer from Intermediate 6S

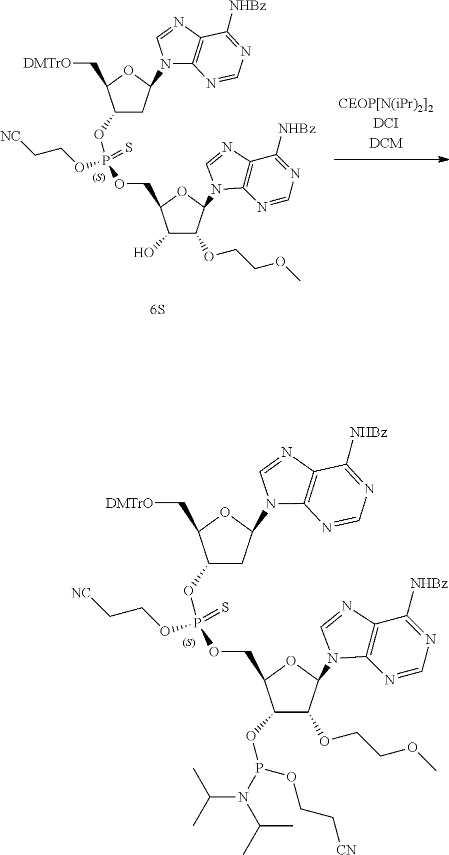

Preparation of Example 20 Dimer: To a solution of 6S (3.2 g, 2.5 mmol) in DCM (30 mL) was added DCI (250 mg, 2.1 mmol) and CEP[N(iPr)₂]₂ (903 mg, 3.0 mmol) under N₂. The mixture was stirred at 25° C. for 2.5 h. LCMS showed 6S was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in Example 20 dimer (2.0 g, 1.4 mmol, 56% yield) as a white solid. ESI-LCMS: m/z 1418.3 [M+H]⁻ 1H NMR (400 MHZ, DMSO-d6) δ 8.66 (d, J=1.6 Hz, 2H), 8.56 (dd, J=4.3, 3.3 Hz, 2H), 8.12-7.90 (m, 4H), 7.64 (tdd, J=6.8, 5.2, 2.6 Hz, 2H), 7.55 (td, J=8.0, 6.4 Hz, 4H), 7.45-7.31 (m, 2H), 7.28-7.13 (m, 7H), 6.88-6.66 (m, 4H), 6.47 (td, J=7.0, 3.4 Hz, 1H), 6.19 (dd, J=14.1, 5.3 Hz, 1H), 4.94 (dt, J=15.5, 5.1 Hz, 1H), 4.52-4.27 (m, 4H), 4.16 (ddq, J=8.5, 5.6, 2.8 Hz, 2H), 3.97-3.58 (m, 13H), 3.50-3.35 (m, 2H), 3.24 (ddd, J=20.5, 17.3, 10.3 Hz, 3H), 3.11 (d, J=3.2 Hz, 3H), 2.97-2.73 (m, 4H), 1.18 (ddd, J=12.4, 6.7, 2.2 Hz, 12H). ³¹P NMR: (400 MHZ, DMSO-d₆): δ 149.69, 149.17, 66.51, 66.48.

### Example 21: Preparation of Example 21 Dimer from Intermediate 6S

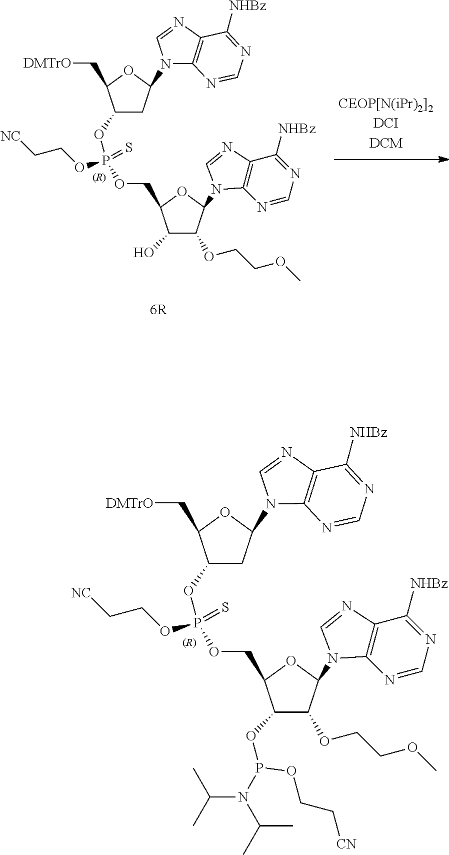

Preparation of Example 21 Dimer: To a solution of 6R (3.0 g, 2.3 mmol) in DCM (30 mL) was added DCI (235 mg, 1.9 mmol) and CEP[N(iPr)₂]₂ (845 mg, 2.8 mmol) under N₂. The mixture was stirred at 25° C. for 2.5 h. LCMS showed 6R was consumed completely. The product was extracted with DCM. The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in Example 21 dimer (1.9 g, 1.3 mmol, 56% yield) as a white solid. ESI-LCMS: m/z 1418.3 [M−H]⁻; ¹H NMR (400 MHZ, DMSO-d₆) δ 11.14 (d, J=9.8 Hz, 2H), 8.69 (d, J=1.7 Hz, 2H), 8.49 (t, J=2.8 Hz, 2H), 8.04-7.84 (m, 4H), 7.57 (tdt, J=6.2, 3.0, 1.4 Hz, 2H), 7.53-7.40 (m, 4H), 7.35-7.23 (m, 2H), 7.22-7.05 (m, 7H), 6.82-6.63 (m, 4H), 6.14 (dd, J=12.7, 5.4 Hz, 1H), 5.57-5.11 (m, 1H), 4.86 (dt, J=17.2, 5.2 Hz, 1H), 4.79-4.67 (m, 1H), 4.46-4.18 (m, 4H), 4.13 (ddp, J=9.0, 6.1, 3.0 Hz, 2H), 3.90-3.51 (m, 13H), 3.33 (dt, J=9.5, 4.7 Hz, 2H), 3.26-3.11 (m, 3H), 3.04 (d, J=3.6 Hz, 3H), 2.82 (t, J=5.8 Hz, 2H), 2.74 (tt, J=6.2, 2.2 Hz, 2H), 1.26-1.00 (m, 13H). ³¹P NMR: (400 MHz, DMSO-d₆): δ 149.70, 149.367, 66.51, 66.44.

### Example 22: Comparison of ASO-6 and ASO-139 with Control ASO-1

In order to identify additional antisense oligonucleotides (ASOs), ASO-6 and ASO-139 were compared to the ASO control, ASO-1.

The efficacy of ASO-6 and ASO-139 to activate RNase H-mediated degradation of the target HBV RNA was determined by performing the EC₅₀ assay as described in Example 2. The EC₅₀ assay as described was used to determine the concentration of ASO required to reduce the transcript levels by 50% in relation to an untreated cell control. Any changes less than two- to three-fold difference from the control ASO was considered similar to the RNase H activation seen utilizing the control ASO-1. ASO-6 and ASO-139 exhibited a reduction in EC₅₀ value as compared to ASO-1, however, the reduction for ASO-139 was approximately 5-fold more than for ASO-6 (Table 5).

The ability of ASO-6 and ASO-139 to trigger cell death was determined as described in Example 2. The CC₅₀ was used to determine the concentration of the ASO as compared to the ASO-1 control to reduce the cell population by 50%, with an increased CC₅₀ as compared to the control representing a decreased cytotoxicity. ASO-6 and ASO-139 had a CC₅₀ comparable to the ASO-1 control and have the same level of toxicity (Table 5).

To assess ASO-6 and ASO-139 ability to reduce HBV infections in cells the TLR8 assay as described in Example 2 was used to evaluate activation of the TLR8 pathway. ASO-6 and ASO-139 values were normalized to the level of TLR8 activation of the ASO-1. ASO-6 increased activation of TLR8 and ASO-139 decreased activation (Table 5).

To determine if ASO-6 and ASO-139 activated cell death mechanisms when transfected into cells, the caspase assay as described in Example 2 was performed. The values of ASO-6 and ASO-139 were normalized to the level of caspase activity detected with the control ASO-1, with ASO-6 having lower caspase levels compared to the ASO-1 control and ASO-139 having higher level of caspases comparing with ASO-1 (Table 5).

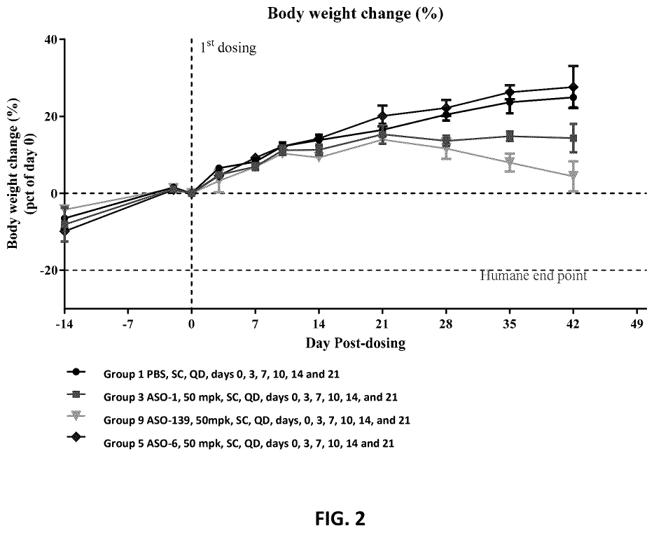

The effect of the ASO-1, ASO-6, and ASO-139 on the body weight of AAV-HBV infected hTLR8 KI mice was determined. Mice were evaluated for percentage body weight change. Group 1 (control) were injected with PBS, Group 3 with 50 mg/kg of ASO-1, Group 9 with 50 mg/kg of AS0-139, and Group 5 with 50 mg/kg of ASO-6 once a day on days 0, 3, 7, 14, and 21. Mice in Group 5 that were treated with ASO-6 tolerated treatment better than Group 3 (ASO-1) and Group 9 (ASO-139) (FIG. 2). ASO-6 had no body weight loss comparing with vehicle control group at the end of the study.

TABLE 5
Assay Results for ASO-1, ASO-6, and ASO-139

| | EC50 | CC50 | hTLR8 Fold vs. | Caspase Activity: Fold vs. |
|---|---|---|---|---|
| ASO | (nM) | (nM) | ASO-1 | ASO-1 at 167 nM |
| ASO-6 | 5.8 | >167 | 1.5 | 0.86 |
| ASO-1 | 8.6 | >167 | 1.0 | 1.0 |
| ASO-139 | 1.8 | >167 | 0.8 | 1.9 |

### Example 23: 5′ Mono GalNAc ASO Improves Liver/Kidney Ratio

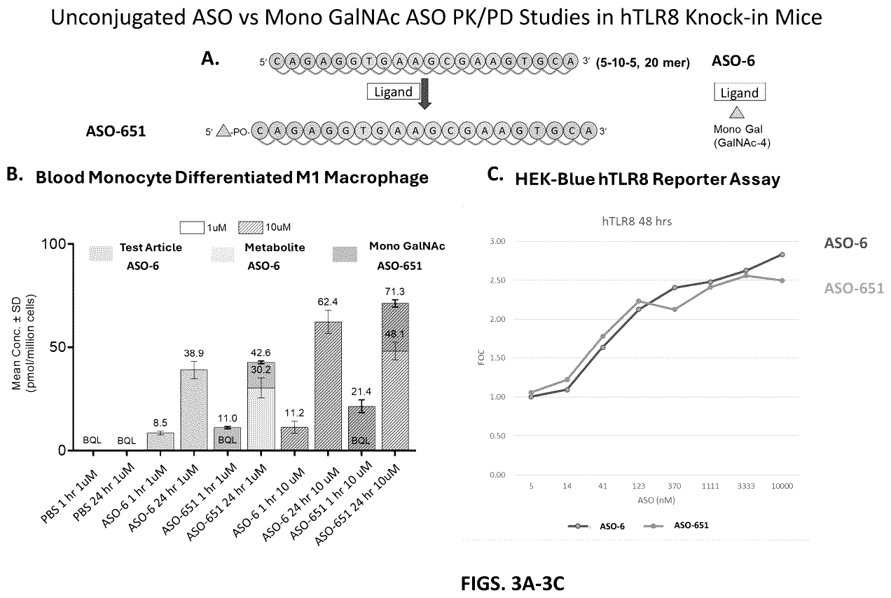

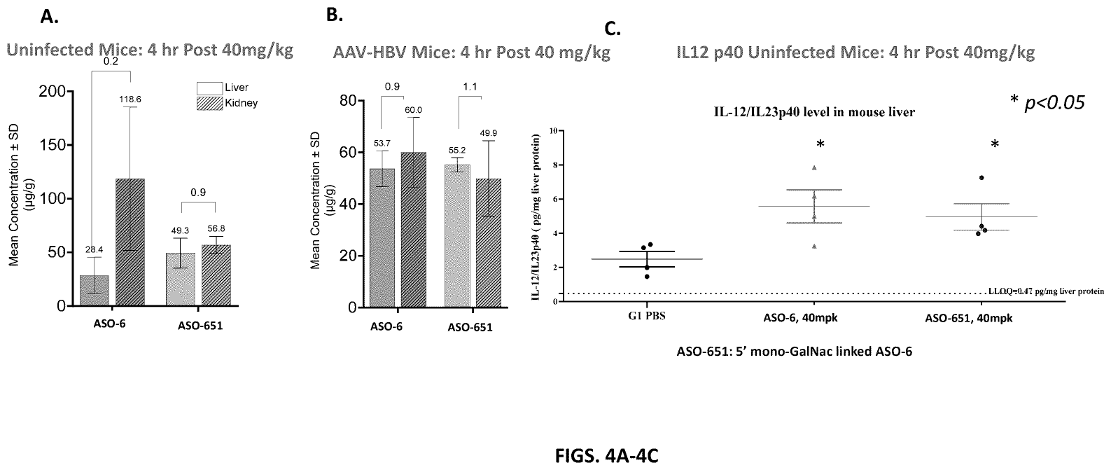

To assess the effects of GalNAc conjugate a mono GalNAc-4 was attached to ASO-6 5′ to create ASO-651 (FIG. 3A). To assess the uptake of both ASO-6 and ASO-651 in macrophages, the concentrations of ASO-651 and ASO-6 (metabolite of ASO-651) were determined in blood monocyte differentiated M1 macrophages. Results demonstrated that there was a gradual uptake of both ASO-6 and ASO-651, with removal of GalNAc occurring in macrophages being time dependent. (FIG. 3B). The ability of ASO-6 and ASO-651 to activate human TLR8 receptor in HEK-Blue hTLR8 cells was assessed as described in Example 2 at differing concentrations. The hTLR8 activity was comparable between ASO-6 and ASO-651 (FIG. 3C). The effects of 5′ Mono GalNAc (ASO-651) in delivering active drug (metabolite ASO-6) to two major organs (liver and Kidney) was studied in AAV-HBV infected hTLR8 knock in mice and uninfected human TLR8 knock in mice 4 hours post single 40 mg/kg dose. 5′ mono GalNAc improved the liver/kidney ration in the range of 22% to 350% comparing with unconjugated ASO (ASO-6). (FIG. 4A-4B). For pharmacodynamic effects, the IL-12 p40 profile was also assessed in uninfected hTLR8 knock in mice. 5′mono GalNAc (ASO-651) achieved the same IL-12 p40 induction as the unconjugated ASO (ASO-6) (FIG. 4C).

### Example 24: ASOs with Locked Nucleic Acid LNA Modification and No LNA Modification

ASOs with LNA modifications and no LNA modifications were prepared based on the parent modification, ASO-6. ASO-182 contains LNA modifications at positions 3 and 18, ASO-222 contains LNA modifications at positions 16, 18, and 20, ASO-114 and ASO-153 that contain no LNA modifications.

ASO-182, ASO-222, ASO-114 and ASO-153 were subjected to the assays described in Example 2 to determine RNAse H-mediated degradation, cytotoxicity, TLR8 pathway activation, and caspase activation (Table 6-9) and compared to control ASO-1.

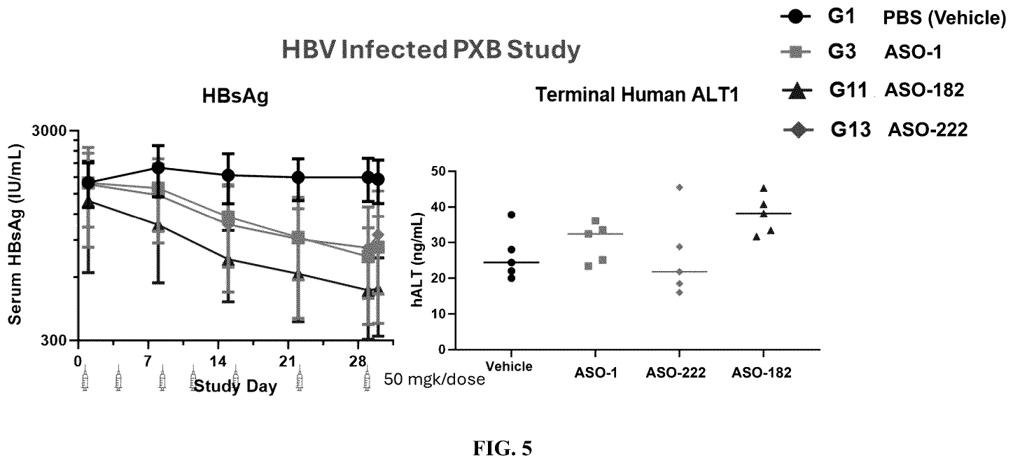

The effect of LNA modified ASO's, ASO-182 and ASO-222, on HBsAg and Terminal Human ALT1 was evaluated in HBV infected PXB mice. Mice were injected with 50 mg/kg per dose of PBS or ASO over a time course of 28 days as depicted in FIG. 5. Group 1 received PBS, Group 3 received ASO-1, Group 11 received ASO-182, and Group 13 received ASO-222. Serum HBsAg and hALT levels were measured at various time points. Results demonstrate the ASO-182 was more potent than ASO-1 control via decreased HBsAg levels and higher hALT levels. ASO-222 had similar potency levels compared the ASO-1 control based on similar HBsAg levels yet had slightly less effect on hALT levels. (FIG. 5).

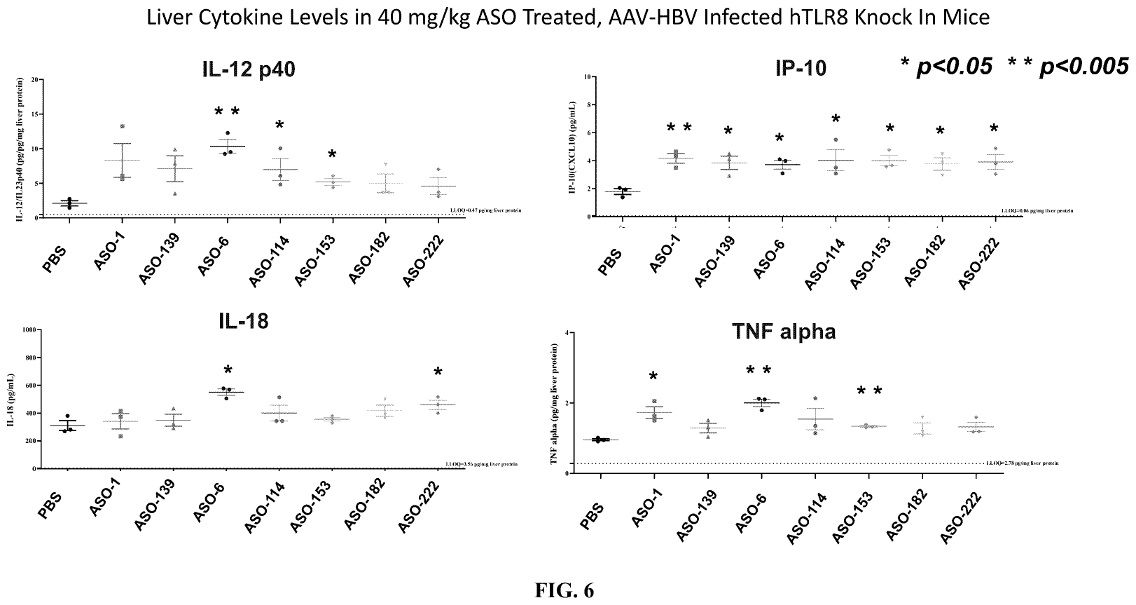

The effect of ASO-1, ASO-139, ASO-6, ASO-114, ASO-153, ASO-182, ASO-222, on liver cytokine proteins was determined in AAV-HBV hTLR8 knock in mice. Mice were treated with a single dose of 40 mg/kg of either ASO-1, ASO-139, ASO-6, ASO-114, ASO-153, ASO-182, or ASO-222, with a n of 3 per treatment group. IL-12 p40, IP-10, IL-18, and TNG-alpha protein levels were tested after treatment (FIG. 6).

TABLE 6
Assay Results for ASO-1 and ASO-182

| | | | | | Caspase | Caspase |
|---|---|---|---|---|---|---|
| | | | | | Activity: Fold | Activity: Fold |
| | LNA | EC50 | CC50 | hTLR8 Fold | vs. ASO-1 at | vs. ASO-1 at |
| ASO | Modification | (nM) | (nM) | vs. ASO-1 | 167 nM | 56 nM |
| ASO-182 | Pos 3 and 18 | 3.3 | >500 | 1.52 | 1.2 | 1.1 |
| ASO-1 | | 10.95 | >500 | 1.0 | 1.0 | 1.0 |

TABLE 7
Assay Results for ASO-1 and ASO-222

| | | | | | Caspase | Caspase |
|---|---|---|---|---|---|---|
| | | | | | Activity: Fold | Activity: Fold |
| | LNA | HepG2.2.15 | HepG2.2.15 | hTLR8 Fold | vs. ASO-1 at | vs. ASO-1 at |
| ASO | Modification | EC50 (nM) | CC50 (nM) | vs. ASO-1 | 167 nM | 56 nM |
| ASO-222 | Pos 16, 18, and 20 | 1.233 | >500 | 1.5 | 1.0 | 1.2 |
| ASO-1 | | 6.001 | 217.9 | 1.0 | 1.0 | 1.0 |

TABLE 8
Assay Results for ASO-1 and ASO-114

| | | | | | Caspase | Caspase |
|---|---|---|---|---|---|---|
| | | | | | Activity: Fold | Activity: Fold |
| | LNA | HepG2.2.15 | HepG2.2.15 | hTLR8 Fold | vs. ASO-1 at | vs. ASO-1 at |
| ASO | Modification | EC50 (nM) | CC50 (nM) | vs. ASO-1 | 167 nM | 56 nM |
| ASO-114 | None | 5.767 | >500 | 1.5 | 0.87 | 0.71 |
| ASO-1 | | 6.001 | 217.9 | 1.0 | 1.0 | 1.0 |

TABLE 9
Assay Results for ASO-1 and ASO-153.

| | | | | | Caspase |
|---|---|---|---|---|---|
| | OMe | HepG2.2.15 | HepG2.2.15 | hTLR8 Fold | Activity: Fold |
| ASO | Modification | EC50 (nM) | CC50 (nM) | vs. ASO-1 | vs. ASO-1 |
| ASO-153 | OMe_Pos- | 10.39 | >500 | 1.73 | 1.1 |
| | 16, 18 & 20 | | | | |
| ASO-1 | | 8.6 | >167 | 1 | 1 |

### Example 25: ASOs Two LNA Walk Set Based on ASO-6

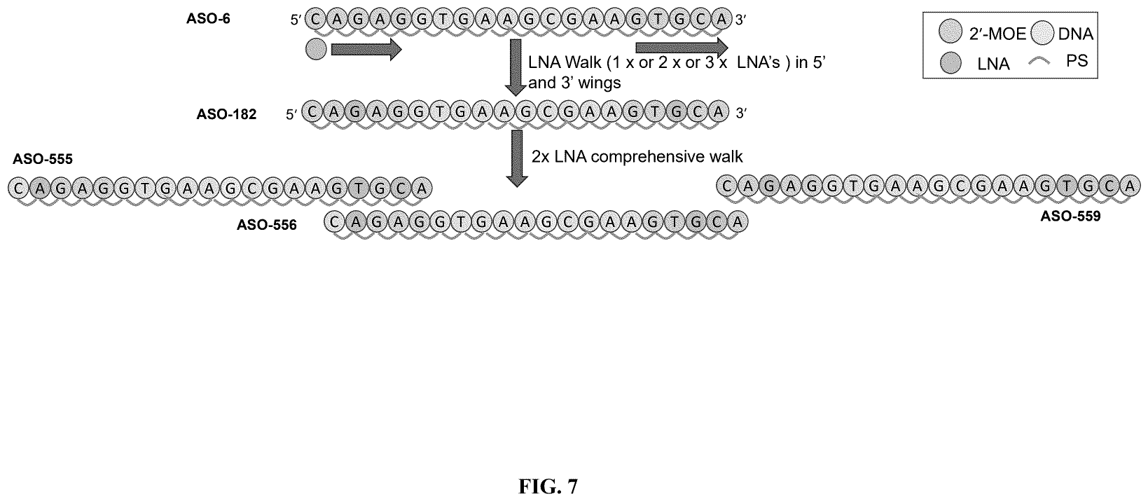

The ASO-6 parental ASO was selected as the starting sequence for preparing a set of ASO with two LNA walk modification to identify better patterns on ASO-6. An exemplary method for creating two LNA walk modifications is disclosed in FIG. 7. ASO-545, ASO-546, ASO-547, ASO-548, ASO-549, ASO-550, ASO-551, ASO-552, ASO-553, ASO-554, ASO-555, ASO-556, ASO-557, ASO-558, ASO-559, ASO-560, ASO-561, and ASO-578 was prepared and subjected to the assays described in Example 2 to determine RNAse H-mediated degradation, cytotoxicity, TLR8 pathway activation, and caspase activation as compared to control ASO-1 (Table 10).

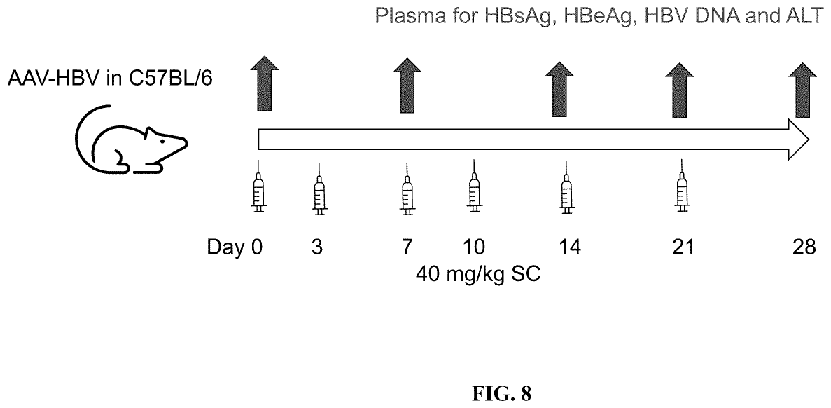

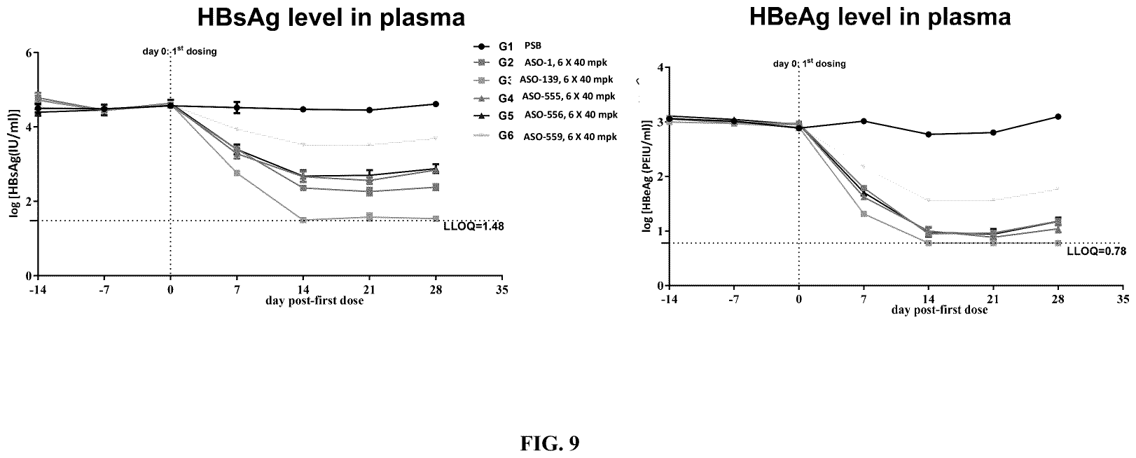

The effect of LNA modified ASO's, ASO-555, ASO-556, and ASO-559, on HBsAg levels in plasma and HBeAg levels in plasma were evaluated in an AAV-HBV C57BL/6 mouse model. Mice were injected with 40 mg/kg ASOs on days 0, 3, 7, 10, 14, and 21. On days predose, 7, 14, 21 and 28, plasma was collected and tested for HBsAg, HBeAg, HBV DNA and ALT. On day 28, liver, kidney and plasma were also collected from each mouse (FIG. 8). Group 1 was injected with PBS, Group 2 with ASO-1 control, Group 3 with ASO-139 control, Group 4 with ASO-555, Group 5 with ASO-556, and Group 6 with ASO-559. ASO-555 and ASO-556 had similar reductions in HBeAg levels as ASO-1 and ASO-139 controls. For HBsAg, ASO-555 and ASO-556 had similar reductions as ASO-1, ASO-139 had deeper HBsAg knock down than the rest (FIG. 9).

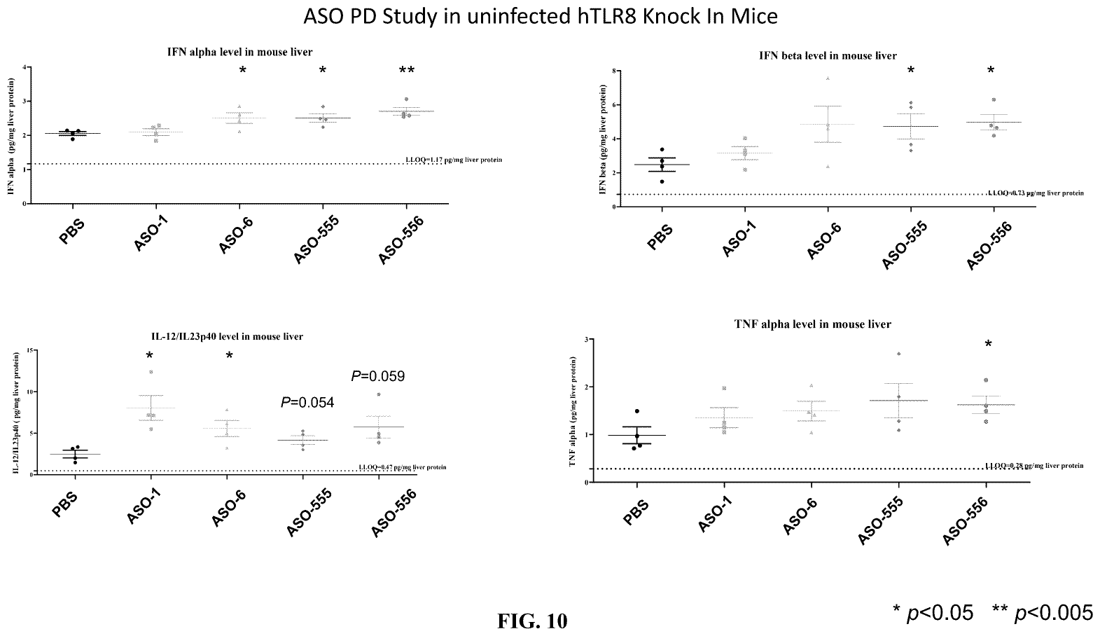

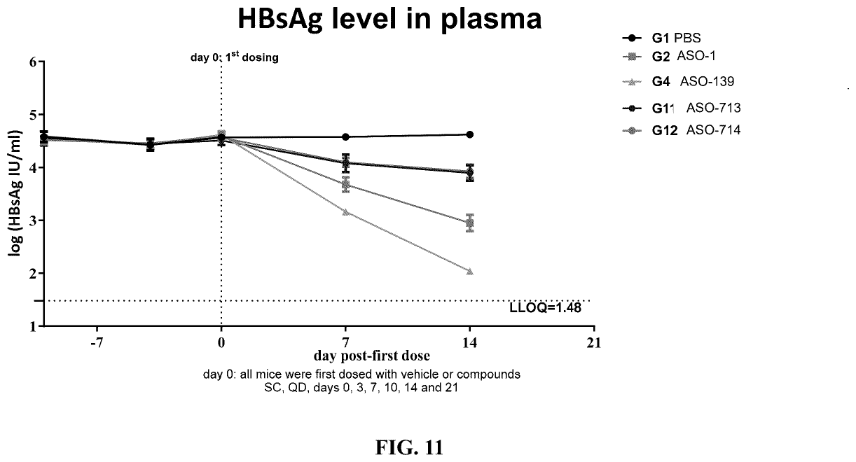

The effect of ASO-1 (control), ASO-6 (control), ASO-555, and ASO-556 on liver cytokine proteins was determined in AAV-HBV hTLR8 knock in mice. Mice were treated with a single dose of 40 mg/kg of either PBS, ASO-1, ASO-6, ASO-555, or ASO-556, with a n of 3 per treatment group. IFN alpha, IFN beta, IL-12/IL23 p40, and TNF alpha protein levels were tested after treatment. Overall these ASOs had similar patterns of cytokine induction (FIG. 10).

TABLE 10
Assay Results for Two LNA walk set.

| | | Caspase | Caspase | | | |
|---|---|---|---|---|---|---|
| | | Activity: | Activity: | | | |
| | | Fold vs. | Fold vs. | | | |
| | LNA | ASO-1 at | ASO-1 at | hTLR8 Fold | HepG2.2.15 | HepG2.2.15 |
| ASO | Modification | 167 nM | 56 nM | vs. ASO-1 | EC50 (nM) | CC50 (nM) |
| ASO-545 | LNA Pos-1_2 | 1.38 | 0.73 | 1.29 | 4.32 | >167 |
| ASO-546 | LNA Pos-1_3 | 0.90 | 1.02 | 1.19 | 3.70 | >167 |
| ASO-547 | LNA Pos-1_4 | 0.85 | 1.18 | 1.05 | 4.06 | >167 |
| ASO-548 | LNA Pos-1_5 | 1.25 | 0.72 | 0.91 | 3.28 | >167 |
| ASO-549 | LNA Pos-1_16 | 1.10 | 1.13 | 1.11 | 4.51 | >167 |
| ASO-550 | LNA Pos-1_18 | 0.90 | 0.81 | 1.15 | 3.66 | >167 |
| ASO-551 | LNA Pos-1_20 | 1.13 | 0.77 | 1.04 | 3.48 | >167 |
| ASO-552 | LNA pos-2_3 | 1.11 | 0.92 | 1.49 | 3.35 | >167 |
| ASO-553 | LNA Pos-2_5 | 1.14 | 1.64 | 1.14 | 3.20 | >167 |
| ASO-554 | LNA Pos-2_16 | 0.87 | 1.29 | 1.43 | 4.40 | >167 |
| ASO-555 | LNA Pos-2_17 | 0.81 | 1.33 | 1.91 | 1.76 | >167 |
| ASO-556 | LNA Pos-2_18 | 0.64 | 0.90 | 1.98 | 3.07 | >167 |
| ASO-557 | LNA Pos-2_19 | 0.87 | 1.09 | 1.54 | 1.25 | >167 |
| ASO-558 | LNA Pos-3_4 | 1.41 | 1.41 | 0.93 | 3.42 | >167 |
| ASO-559 | LNA Pos-3_17 | 1.17 | 1.17 | 1.69 | 2.04 | >167 |
| ASO-560 | LNA Pos-3_19 | 0.81 | 1.23 | 1.60 | 2.84 | >167 |
| ASO-561 | LNA Pos-3_20 | 1.14 | 0.64 | 1.38 | 3.27 | >167 |
| ASO-1 | N/A | 1 | 1 | 1 | 3.4 | >167 |

### Example 26: ASOs Full LNA Walk Set Based on ASO-6

ASO-6 was selected as the starting sequence for preparing a set of ASOs with full LNA walk modifications, ASO-562, ASO-563, ASO-564, ASO-565, ASO-566, ASO-567, ASO-568, ASO-569, ASO-570, ASO-571, ASO-572, ASO-573, ASO-574, ASO-575, ASO-576, and ASO-577.

The ASOs were subjected to the assays described in Example 2 to determine RNAse H-mediated degradation, cytotoxicity, TLR8 pathway activation, and caspase activation as compared to control ASO-1 (Table 11).

TABLE 11
Assay Results for full LNA walk set.

| | | Caspase | Caspase | | | |
|---|---|---|---|---|---|---|
| | | Activity: | Activity: | | | |
| | | Fold vs. | Fold vs. | | | |
| | LNA | ASO-1 at | ASO-1 at | hTLR8 Fold | HepG2.2.15 | HepG2.2.15 |
| ASO | Modification | 167 nM | 56 nM | vs. ASO-1 | EC50 (nM) | CC50 (nM) |
| ASO-562 | LNA Pos-4_5 | 3.23 | 14.10 | 0.71 | 2.98 | >167 |
| ASO-563 | LNA Pos-4_16 | 2.34 | 6.30 | 0.90 | 3.43 | >167 |
| ASO-564 | LNA Pos-4_17 | 2.66 | 11.23 | 0.83 | 1.52 | 23.11 |
| ASO-565 | LNA Pos-4_18 | 2.13 | 5.45 | 0.95 | 2.64 | >167 |
| ASO-566 | LNA Pos-4_19 | 2.54 | 9.21 | 0.85 | 2.58 | >167 |
| ASO-567 | LNA Pos-5_17 | 2.90 | 13.27 | 0.67 | 1.21 | >167 |
| ASO-568 | LNA Pos-5_19 | 2.98 | 11.92 | 0.59 | 1.64 | >167 |
| ASO-569 | LNA Pos-5_20 | 2.89 | 10.73 | 0.80 | 2.33 | >167 |
| ASO-570 | LNA Pos-16_17 | 1.69 | 7.94 | 1.34 | 1.70 | >167 |
| ASO-571 | LNA Pos-16_19 | 1.64 | 5.44 | 1.09 | 1.83 | >167 |
| ASO-572 | LNA Pos-16_20 | 1.65 | 5.19 | 1.21 | 2.88 | >167 |
| ASO-573 | LNA Pos-17_18 | 1.83 | 6.18 | 1.16 | 1.43 | >167 |
| ASO-574 | LNA Pos-17_19 | 2.17 | 8.88 | 1.25 | 1.23 | >167 |
| ASO-575 | LNA Pos-17_20 | 1.95 | 8.12 | 1.39 | 1.27 | >167 |
| ASO-576 | LNA Pos-18_19 | 2.06 | 5.47 | 1.24 | 1.64 | >167 |
| ASO-577 | LNA Pos-18_20 | 1.78 | 4.31 | 1.29 | 1.77 | >167 |
| ASO-1 | N/A | N/A | N/A | N/A | 4.53 | >167 |
| ASO-6 | N/A | 0.85 | 0.30 | 1.13 | 8.37 | >167 |

### Example 27: ASOs Replacing LNA with GuNA

To assess the effect of GuNA, LNA in ASO-182 and ASO-222 was replaced with GuNA to create ASO-784, ASO-785, ASO-752, and ASO-831. Additional ASO's were created replacing single or double LNA from various parental constructs with GuNA to create ASO-712, ASO-713, ASO-714, and ASO-715. The ASOs were subjected to the assays described in Example 2 to determine RNAse H-mediated degradation, cytotoxicity, TLR8 pathway activation, and caspase activation as compared to control ASO-1 and/or ASO-6 (Table 12-13).

TABLE 12
Assay Results for ASOs with GuNA

| | Caspase Activity: | Caspase Activity: | | | |
|---|---|---|---|---|---|
| | Fold vs. ASO-1 at | Fold vs. ASO-1 at | hTLR8 Fold | HepG2.2.15 | HepG2.2.15 |
| ASO | 167 nM | 56 nM | vs. ASO-1 | EC50 (nM) | CC50 (nM) |
| ASO-784 | 1.70 | 3.61 | 1.37 | 1.67 | >167 |
| ASO-785 | 1.73 | 2.62 | 0.86 | 1.39 | >167 |
| ASO-752 | 1.75 | 3.45 | 0.90 | 1.55 | >167 |
| ASO-831 | 0.90 | 0.36 | 1.57 | 2.3 | >167 |
| ASO-222 | 1.58 | 2.66 | 1.02 | 2.29 | >167 |
| ASO-1 | 1 | 1 | 1 | 4.31 | >167 |

TABLE 13
Assay Results for ASOs with GuNA

| | | Caspase | Caspase | | | |
|---|---|---|---|---|---|---|
| | | Activity: Fold | Activity: Fold | hTLR8 | | |
| | GuNA | vs. ASO-1 at | vs. ASO-1 at | Fold vs. | HepG2.2.15 | HepG2.2.15 |
| ASO | Position | 167 nM | 56 nM | ASO-1 | EC50 (nM) | CC50 (nM) |
| ASO-712 | 17 | 2.21 | 3.11 | 0.87 | 8.03 | >167 |
| ASO-713 | 18 | 0.59 | 0.16 | 1.05 | 1.31 | >167 |
| ASO-714 | 19 | 0.69 | 0.43 | 1.00 | 2.35 | >167 |
| ASO-715 | 2, 19 | 1.65 | 0.92 | 1.24 | 3.37 | >167 |
| ASO-6 | N/A | N/A | N/A | 1.06 | 11.579 | >167 |
| ASO-1 | N/A | 1 | 1 | 1 | 10.33 | >167 |

The effect of ASO-713 and ASO-714 on HBsAg levels in plasma was tested in mice. Mice were injected with either PBS (Group 1) or 6×25 mg/kg ASOs including ASO-1 (Group 2), ASO-139 (Group 4), ASO-713 (Group 11), and ASO-714 (Group 12), on days 0, 3, 7, 10, 14, and 21, with ASO-713 and ASO-714 showing suboptimal potency in vivo potency as compared to ASO-1 and ASO-139 (FIG. 11)

### Example 28: ASOs 2′-OMe Abasic Monomer Walk Based on ASO-1

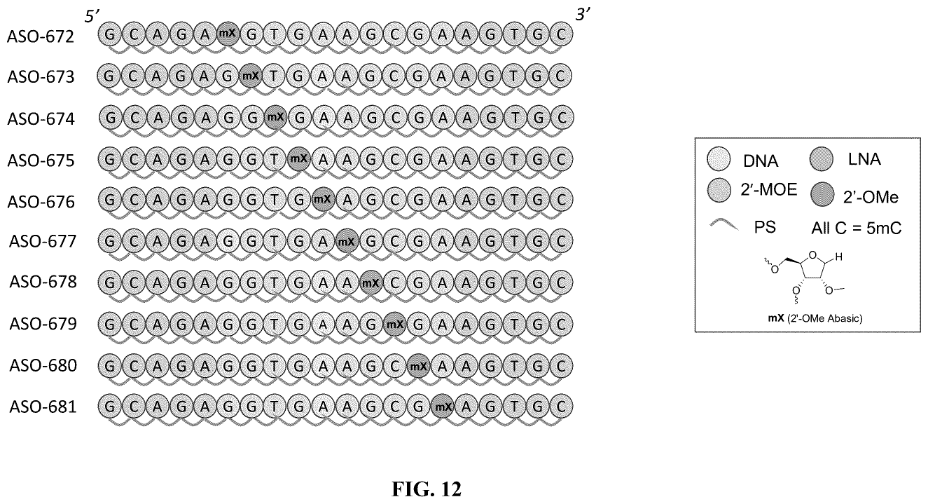

To assess the effect of 2′-OMe abasic monomer modifications in the gap regions, ASO-1 was used as the parental ASO and ASO-672, ASO-673, ASO-674, ASO-675, ASO-676, ASO-677, ASO-678, ASO-679, ASO-680, and ASO-681 were prepared (FIG. 12). The ASOs were subjected to the assays described in Example 2 to determine RNAse H-mediated degradation, cytotoxicity, TLR8 pathway activation, and caspase activation as compared to control ASO-1 (Table 14).

TABLE 14
Assay Results for ASOs with 2′OMe Abasic modification (mX)

| | | Caspase | Caspase | | | |
|---|---|---|---|---|---|---|
| | | Activity: Fold | Activity: Fold | hTLR8 | | |
| | | vs. ASO-1 at | vs. ASO-1 at | Fold vs. | HepG2.2.15 | HepG2.2.15 |
| ASO | Modification | 167 nM | 56 nM | ASO-1 | EC50 (nM) | CC50 (nM) |
| ASO-672 | mX 6 | 0.64 | 0.47 | 0.96 | 7.99 | >167 |
| ASO-673 | mX 7 | 0.58 | 0.52 | 1.01 | 15.2 | >167 |
| ASO-674 | mX 8 | 0.73 | 0.33 | 0.96 | 12.78 | >167 |
| ASO-675 | mX 9 | 0.82 | 0.41 | 0.81 | 21.65 | >167 |
| ASO-676 | mX 10 | 0.63 | 0.34 | 0.89 | 15.08 | >167 |
| ASO-677 | mX 11 | 0.58 | 0.34 | 0.95 | 9.2 | >167 |
| ASO-678 | mX 12 | 0.56 | 0.09 | 0.61 | 42.78 | >167 |
| ASO-679 | mX 13 | 0.59 | 0.24 | 0.75 | 22.19 | >167 |
| ASO-680 | mX 14 | 0.70 | 0.49 | 0.67 | 18.05 | >167 |
| ASO-681 | mX 15 | 0.95 | 0.53 | 0.61 | 23.76 | >167 |
| ASO-1 | N/A | 1 | 1 | 1 | 8.6 | >167 |
| ASO-139 | N/A | 1.9 | 1.36 | 0.8 | 1.8 | >167 |

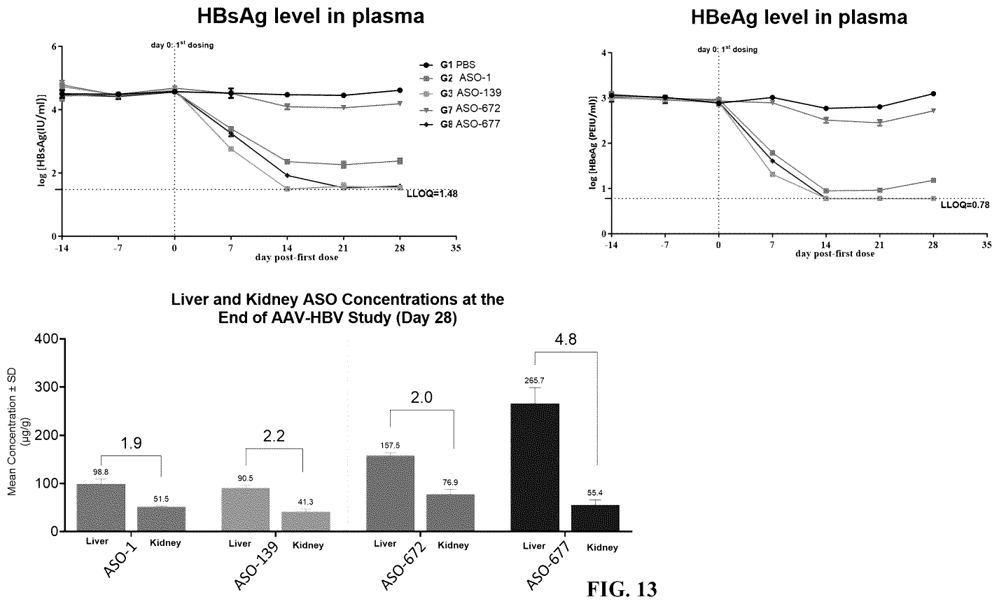

The effect of ASO-672 and ASO-677 on HBsAg levels in plasma and HBeAg levels in plasma were evaluated in an AAV-HBV C57BL/6 mouse model as described in Example 25. Group 1 was injected with PBS, Group 2 with ASO-1 control, Group 3 with ASO-139 control, Group 7 with ASO-672 and Group 8 with ASO-677. The ASOs were dosed at 6×40 mg/kg on days 0, 3, 7, 10, 14, 21. ASO-677 has reductions in HBsAg and HBeAg levels deeper than ASO-1 control and comparable to ASO-139 control (FIG. 13). 2′ OMe abasic modification is at position #6 in ASO-672 and at position #11 in ASO-677 resulted similar in vitro potency (EC50 in HepG2.2.15 assay); however the in vivo potency of these ASOs was drastically different and unexpected. ASO-677 is more potent than ASO-1 with no 2′ OMe abasic monomer, and it knocks down HBsAg as much as ASO-139. ASO-672 on the other hand, is mostly inactive in vivo. Interestingly and unexpectedly, incorporating an abasic monomer at position #11, as in ASO-677, resulted in significantly improved liver exposure and liver-to-kidney ratio relative to reference ASOs: ASO-1 and ASO-139. For treating liver disease, ASO-677 has a more desirable PK profile than the reference ASOs ASO-1 and ASO-139.

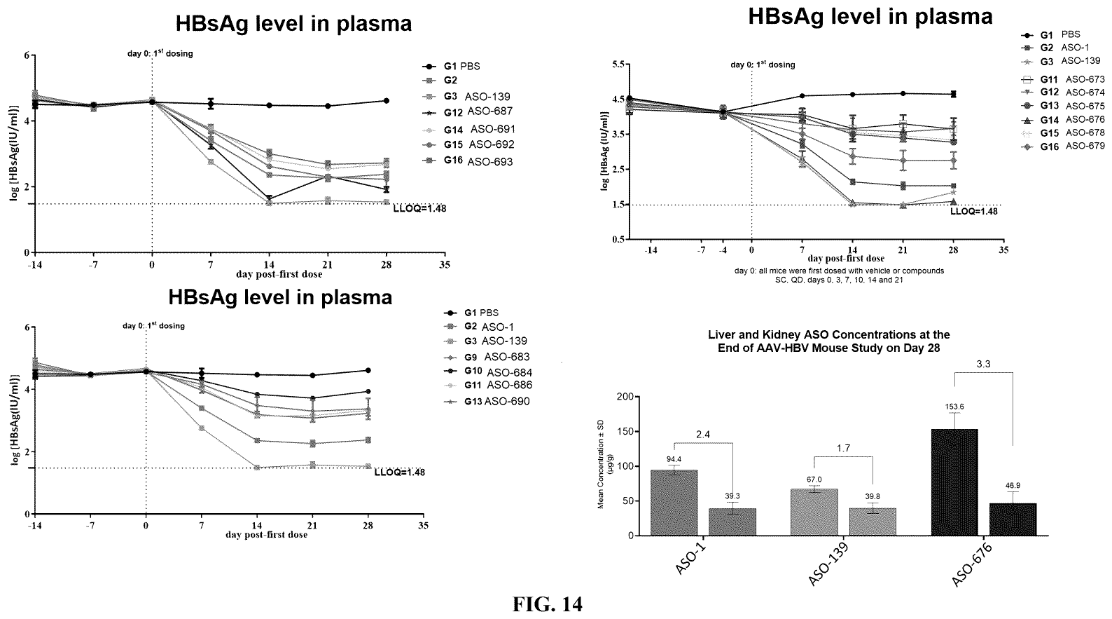

The effect of remaining ASOs (ASO-673, ASO-674, ASO-675, ASO-676, ASO-678 and ASO-679 on HBsAg levels in plasma were evaluated in an AAV-HBV C57BL/6 mouse model as described in Example 25. Group 1 was injected with PBS, Group 2 with ASO-1 control, Group 3 with ASO-139 control, Group 11, 12, 13, 14, 15, 16 with ASO-673, ASO-674, ASO-675, ASO-676, ASO-678 and ASO-679 respectively. The ASOs were dosed at 6×25 mg/kg on days 0, 3, 7, 10, 14, 21. ASO-676 has reductions in HBsAg levels significantly deeper than ASO-1 control and slightly better than ASO-139 control (FIG. 14). The in vitro potency (HepG2.2.15 EC50) of ASO-673, ASO-674 and ASO-676 were quite similar, however ASO-673 and ASO-674 were mostly inactive in vivo while ASO-676 was the most potent ASO in this study, more potent than ASO-1 and ASO-139 controls. Interestingly, incorporating an abasic monomer at position #10, as in ASO-676, resulted in significantly improved liver exposure and liver-to-kidney ratio relative to reference ASOs: ASO-1 and ASO-139. This preferred PK profile which likely contributed to ASO-676's desired in vivo potency. For a drug treating liver disease, this PK profile of ASO-676 was better than those of ASO-1 and ASO-139.

### Example 29: ASOs Mismatch Walk in Gap Based on ASO-1

To assess the effect of mismatch in the gap regions, ASO-1 was used as the parental ASO and ASO-682, ASO-683, ASO-684, ASO-685, ASO-686, ASO-687, ASO-688, ASO-689, ASO-690, ASO-691, ASO-692, and ASO-693 were prepared. The ASOs were subjected to the assays described in Example 2 to determine RNAse H-mediated degradation, cytotoxicity, TLR8 pathway activation, and caspase activation as compared to control ASO-1 (Table 15).

TABLE 15
Assay Results for ASOs with Mismatch

| | | | Caspase | | | |
|---|---|---|---|---|---|---|
| | | Caspase | Activity: | | | |
| | | Activity: Fold | Fold vs. | hTLR8 | | |
| | | vs. ASO-1 at | ASO-1 at | Fold vs. | HepG2.2.15 | HepG2.2.15 |
| ASO | Position | 167 nM | 56 nM | ASO-1 | EC50 (nM) | CC50 (nM) |
| ASO-682 | MMPos9_dA | 1.02 | 0.28 | 1.01 | 10.86 | >167 |
| ASO-683 | MMPos9_d(5m)C | 0.60 | 0.05 | 0.87 | 4.69 | >167 |
| ASO-684 | MMPos9_dT | 0.78 | 0.15 | 0.89 | 5.29 | >167 |
| ASO-685 | MMPos10_d(5m)C | 1.46 | 0.62 | 1.21 | 3.34 | >167 |
| ASO-686 | MMPos10_dG | 0.63 | 0.29 | 1.00 | 11.47 | >167 |
| ASO-687 | MMPos10_dT | 0.60 | 0.68 | 1.04 | 9.91 | >167 |
| ASO-688 | MMPos11_d(5m)C | 0.81 | 0.99 | 1.01 | 8.17 | >167 |
| ASO-689 | MMPos11_dG | 1.58 | 1.69 | 1.17 | 7.93 | >167 |
| ASO-690 | MMPos11_dT | 0.75 | 0.39 | 1.23 | 4.07 | >167 |
| ASO-691 | MMPos12_dA | 0.53 | 0.36 | 1.11 | 20.19 | >167 |
| ASO-692 | MMPos12_d(5m)C | 0.28 | 0.61 | 1.03 | 10.50 | >167 |
| ASO-693 | MMPos12_dT | 0.84 | 0.29 | 1.17 | 5.78 | >167 |
| ASO-1 | N/A | 1 | 1 | 1 | 10.32 | >167 |

The effect of ASO-687, ASO-691, ASO-692, ASO-693, ASO-683, ASO-684, ASO-686, and ASO-690, on HBsAg levels in plasma in were evaluated in an AAV-HBV C57BL/6 mouse model as described in Example 25. Group 1 was injected with PBS, Group 2 with ASO-1 control, Group 3 with ASO-139 control, and other groups were administered with ASO-687, ASO-691, ASO-692, ASO-693, ASO-683, ASO-684, ASO-686, ASO-690, ASO-687, and ASO-691. ASOs were dosed subcutaneously at 6×40 mg/kg on days 0, 3, 7, 10, 14 and 21. Most mismatched ASOs reduced HBsAg levels at different degrees (FIG. 14, left side). The in vivo potency rank order of mismatched ASOs cannot be predicted from their in vitro rank order. ASO-687 was the most potent mismatched ASO in reducing HBsAg in AAV-HBV mouse: potency was between ASO-1 and ASO-139.

### Example 30: ASOs Mismatch Walk in Gap Positions Based on ASO-1

To assess the effect of mismatch in the gap regions, ASO-1 was used as the parental ASO and a mismatch walk of positions 6-8 was performed to prepare ASO-743, ASO-744, ASO-745, ASO-746, ASO-747, ASO-748, ASO-749, ASO-750, and ASO-751. Additionally, a mismatch at positions 13-15 was also performed to prepare ASO-753, ASO-755, ASO-756, ASO-757, ASO-758, ASO-759, ASO-760, ASO-761, and ASO-762. The ASOs were subjected to the assays described in Example 2 to determine RNAse H-mediated degradation, cytotoxicity, TLR8 pathway activation, and caspase activation as compared to control ASO-1 (Table 16). ASO-755 showed an improved caspase profile with similar RNase H and hTLR8 activities (Table 16).

TABLE 16
Assay Results for ASOs with Mismatch in Gap positions

| | | Caspase | | | | |
|---|---|---|---|---|---|---|
| | | Fold over | Caspase Fold | | | hTLRA |
| Mismatch | | ASO-1 | over ASO-1 | EC50 | CC50 | Fold over |
| of ASO-1 | Description | 167 nM | 56 nM | nM | nM | ASO-1 |
| ASO-1 | N/A | 1 | 1 | 10.32 | >167 | |
| ASO-743 | Mismatch at position 6 (A) | 1.17 | 0.64 | 11.10 | >167 | 0.85 |
| ASO-744 | Mismatch at position 6 (5mC) | 2.36 | 2.41 | 9.919 | >167 | 0.54 |
| ASO-745 | Mismatch at position 6 (T) | 1.94 | 2.57 | 7.141 | >167 | 0.71 |
| ASO-746 | Mismatch at position 7 (A) | 0.90 | 2.17 | 5.714 | >167 | 0.90 |
| ASO-747 | Mismatch at position 7 (5mC) | 1.22 | 1.75 | 10.29 | >167 | 0.73 |
| ASO-748 | Mismatch at position 7 (T) | 1.30 | 1.30 | 21.15 | >167 | 0.92 |
| ASO-749 | Mismatch at position 8 (A) | 1.19 | 0.57 | 8.78 | >167 | 0.89 |
| ASO-750 | Mismatch at position 8 (5mC) | 0.66 | 0.34 | 12.05 | >167 | 0.50 |
| ASO-751 | Mismatch at position 8 (G) | 1.07 | 2.53 | 9.96 | >167 | 0.65 |
| ASO-753 | Mismatch at position 15 (G) | 1.65 | 1.07 | 19.33 | >167 | 0.62 |
| ASO-755 | Mismatch at position 15 (T) | 0.82 | 0.86 | 15.79 | >167 | 1.06 |
| ASO-756 | Mismatch at position 15 (5mC) | 1.01 | 0.68 | 28.197 | >167 | 1.16 |
| ASO-757 | Mismatch at position 14 (A) | 0.20 | 0.18 | 51.71 | >167 | 1.05 |
| ASO-758 | Mismatch at position 14 (T) | 0.54 | 0.82 | 30.53 | >167 | 1.13 |
| ASO-759 | Mismatch at position 14 (5mC) | 0.78 | 0.76 | 27.21 | >167 | 0.80 |
| ASO-760 | Mismatch at position 13 (A) | 0.45 | 0.11 | >167 | >167 | 1.11 |
| ASO-761 | Mismatch at position 13 (T) | 0.31 | 0 | 110.72 | >167 | 0.98 |
| ASO-762 | Mismatch at position 13 (G) | 1.12 | 0.41 | 57.53 | >167 | 0.63 |

### Example 31: ASOs 2′-Deoxy Abasic Monomer Walk Based on ASO-1

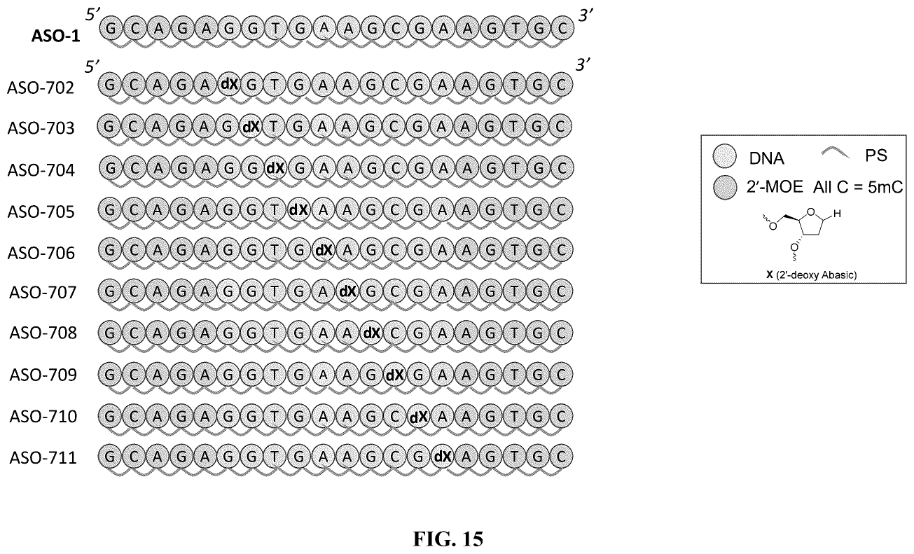

To assess the effect of 2′-deoxy abasic monomer modifications in the gap regions, ASO-1 was used as the parental ASO and ASO-702, ASO-703, ASO-704, ASO-705, ASO-706, ASO-707, ASO-708, ASO-709, ASO-710, and ASO-711 were prepared (FIG. 15). The ASOs were subjected to the assays described in Example 2 to determine RNAse H-mediated degradation, cytotoxicity, TLR8 pathway activation, and caspase activation as compared to control ASO-1 (Table 17). All ASOs show improved caspase profiles. ASO-703 and ASO-709 showed significant loss of RNase H activity. ASO-704, ASO-706, and ASO-707 showed similar RNase H and hTLR8 activity as compared to ASO-1 (Table 17).

TABLE 17
Assay Results for ASOs with 2′-deoxy abasic
monomer modifications in the gap regions

| | | Caspase | | hTLR8 | | |
|---|---|---|---|---|---|---|
| ASO-1 | | Fold over | Caspase Fold | Fold | | |
| 2′deoxy | | ASO-1 | over ASO-1 | over | EC50 | CC50 |
| walk | Position | 167 nM | 56 nM | ASO-1 | nM | nM |
| ASO-702 | 6 | 0.70 | 0.41 | 0.70 | 22.63 | >167 |
| ASO-703 | 7 | 0.12 | 0.11 | 0.80 | >167 | >167 |
| ASO-704 | 8 | 0.83 | 0.15 | 0.92 | 14.53 | >167 |
| ASO-705 | 9 | 0.77 | 0.18 | 1.01 | 28.08 | >167 |
| ASO-706 | 10 | 0.71 | 0.40 | 0.88 | 16.42 | >167 |
| ASO-707 | 11 | 0.68 | 0.17 | 0.94 | 19.16 | >167 |
| ASO-708 | 12 | 0.41 | 0.11 | 0.76 | 61.31 | >167 |
| ASO-709 | 13 | 0.25 | 0.26 | 0.90 | 90.09 | >167 |
| ASO-710 | 14 | 0.59 | 0.43 | 0.75 | 26.13 | >167 |
| ASO-711 | 15 | 0.81 | 0.47 | 0.77 | 39.28 | >167 |
| ASO-1 | | 1 | 1 | 1 | 10.3 | >167 |

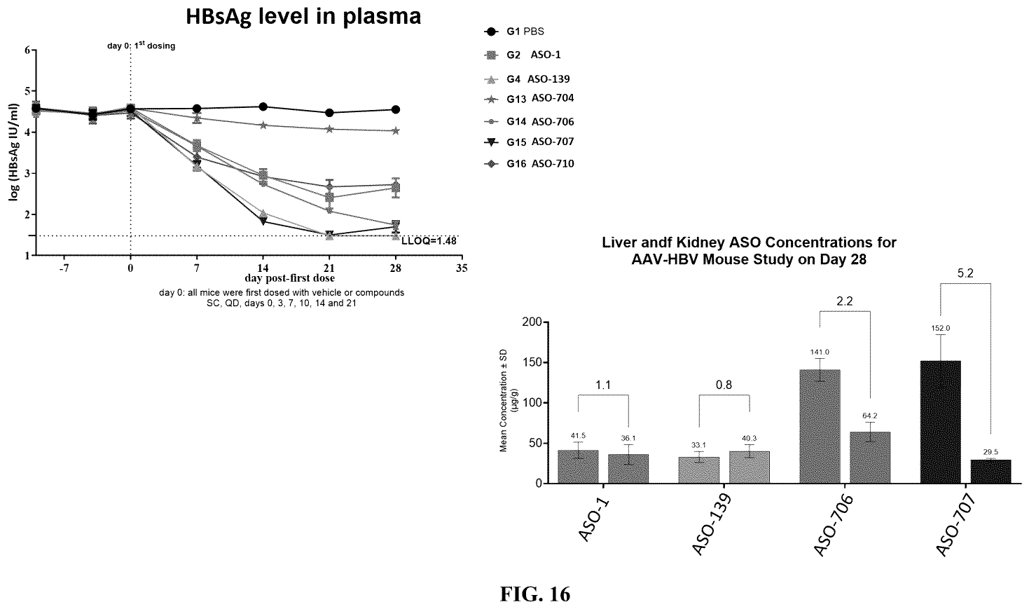

The effect of ASO-704, ASO-706, ASO-707, and ASO-710, on HBsAg levels in plasma were evaluated in an AAV-HBV C57BL/6 mouse model as described in Example 25. Group 1 was injected with PBS, Group 2 with ASO-1 control, Group 3 with ASO-139 control, Group 13 with ASO-704, Group 14 with ASO-706, Group 15 with ASO-707, and Group 16 with ASO-710. Mice were injected in days 0, 3, 7, 10, 14, and 21, with 25 mg/kg. ASO-707 shows similar potency to ASO-139 control, and significantly higher potency than ASO-1 control (FIG. 16). As shown in Table 17, 2′ Deoxy abasic modification at different locations in the gap region improved in vitro safety profile as demonstrated by caspase reduction. However, the impact of such modification on in vitro potency is location dependent. ASO-704, ASO-706, ASO-707 and ASO-710 showed similar or slightly lower in vitro activities compared with ASO-1. As shown in FIG. 16, although ASO-706 and ASO-707 showed similar or slightly lower in vitro activity relative to ASO-1, both of these ASOs significantly outperformed ASO-1 in the AAV-HBV mouse model. Moreover, incorporating a 2′ Deoxy Abasic modification in ASO-706 and ASO-707 appears to significantly improve liver exposure and liver-to-kidney ratio relative to reference ASOs: ASO-1 and ASO-139 (FIG. 16). For treating liver disease, ASO-706 and ASO-707 show PK advantage compared with references ASO-1 and ASO-139.

### Example 32: ASOs 2′-OMe Abasic Monomer Gap and LNA Walk Based on ASO-672

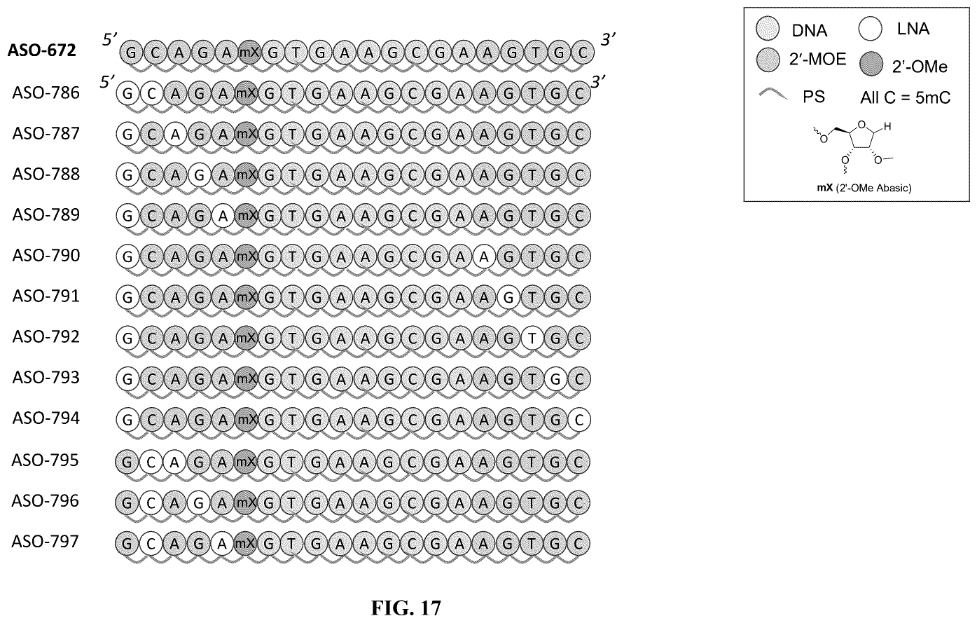

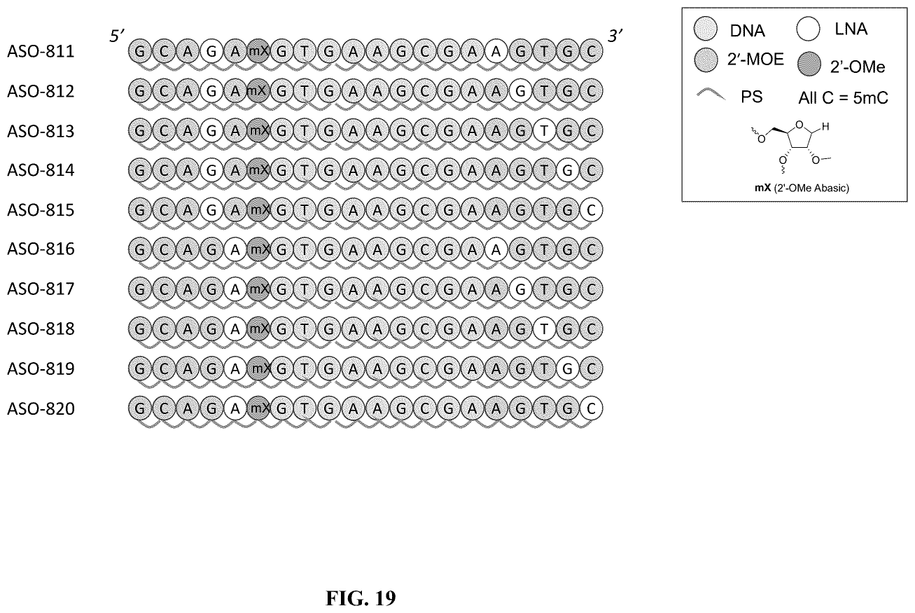

To assess the effect of 2′-OMe abasic monomer modifications in the gap regions and LNA modifications at the wings, ASO-672 as described in Example 28 was used as the parental ASO and ASO-786 to ASO-830, were prepared (FIG. 17-FIG. 20).

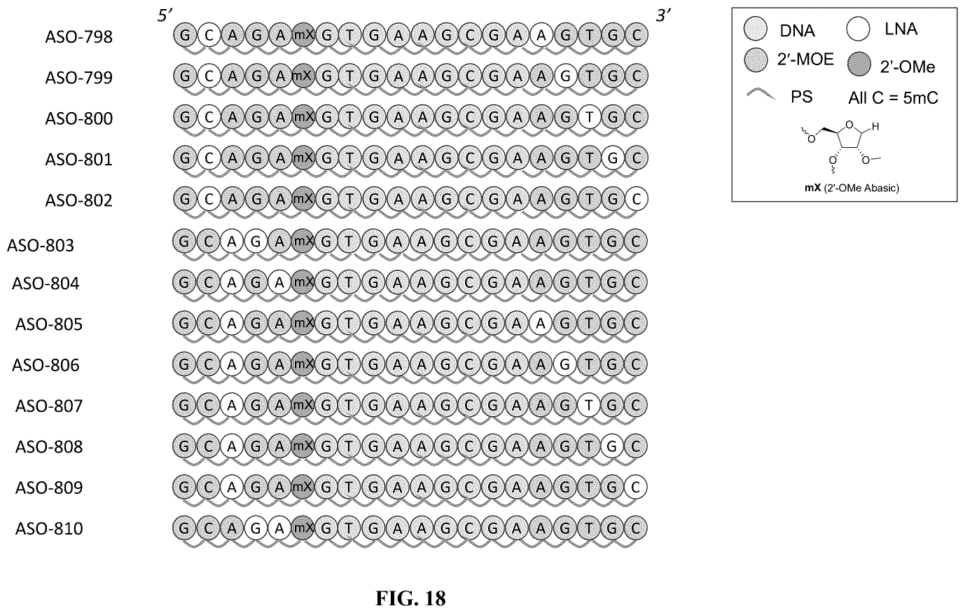

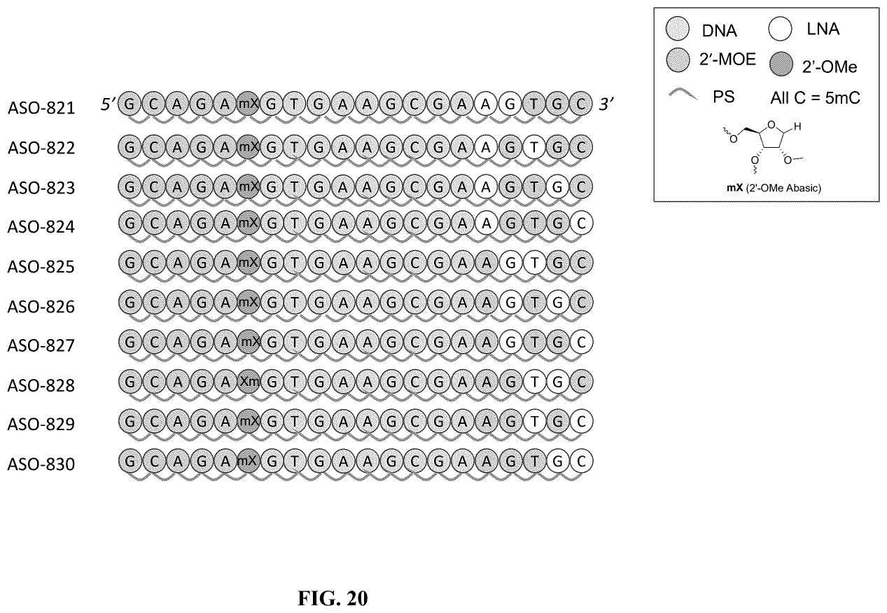

The ASOs were subjected to the assays described in Example 2 to determine RNAse H-mediated degradation, cytotoxicity, TLR8 pathway activation, and caspase activation as compared to control ASO-1 and ASO-672 (FIG. 18 and FIG. 20). Most ASOs showed improved caspase profiles. ASO-790, ASO 791, ASO-793, ASO-794, ASO-796, ASO-806, ASO-807, ASO-812, ASO-817, ASO-818, ASO-819, ASO-822, ASO-825, ASO-828 and ASO-829 showed similar RNase H and as compared to ASO-1 (Tables 18 and 19). ASO-810, ASO-816, ASO-819, ASO-820, ASO-822, ASO-823, ASO-824 and ASO-828 had similar hTLR8 activity as compared to ASO-1.

TABLE 18
Assay Results for ASOs based on ASO 672 with LNA Walk

| | | Caspase | | | | |
|---|---|---|---|---|---|---|
| | | Fold over | Caspase Fold | | | TLR8 |
| ASO-672 + | | ASO-1 | over ASO-1 | EC50 | CC50 | Fold over |
| LNA Walk | Position | 167 nM | 56 nM | nM | nM | ASO-1 |
| ASO-786 | mX 6 LNA 1 2 | 0.82 | 0.3 | 5.84 | >167 | 0.68 |
| ASO-787 | mX 6 LNA 1 3 | 0.66 | 0.37 | 6.49 | >167 | 0.69 |
| ASO-788 | mX 6 LNA 1 4 | 1.24 | 0.27 | 6.68 | >167 | 0.74 |
| ASO-789 | mX 6 LNA 1 5 | 1.07 | 0.05 | 8.00 | >167 | 0.67 |
| ASO-790 | mX 6 LNA 1 16 | 1.19 | 0.06 | 14.76 | >167 | 0.63 |
| ASO-791 | mX 6 LNA 1 17 | 0.93 | 0.11 | 12.31 | >167 | 0.53 |
| ASO-792 | mX 6 LNA 1 18 | 1.65 | 0.58 | 6.78 | >167 | 0.62 |
| ASO-793 | mX 6 LNA 1 19 | 0.76 | −0.27 | 13.39 | >167 | 0.60 |
| ASO-794 | mX 6 LNA 1 20 | 1.39 | 0.50 | 13.19 | >167 | 0.61 |
| ASO-1 | | 1 | 1 | 10.46 | >167 | 1 |

TABLE 19
Assay Results for ASOs based on ASO-672
with 2′-OMe abasic monomer Gap and LNA

| ASO-672 | | | | | | |
|---|---|---|---|---|---|---|
| 2′OMe | | Caspase | | | | |
| abasic | | Fold over | Caspase Fold | | | hTLR8 |
| monomer + | | ASO-1 | over ASO-1 | EC50 | CC50 | Fold over |
| LNA Walk | Position | 167 nM | 56 nM | nM | nM | ASO-1 |
| ASO-796 | mX 6 LNA 2 4 | 0.56 | 0.05 | 10.62 | >167 | 0.64 |
| ASO-797 | mX 6 LNA 2 5 | 0.24 | 0.10 | 5.80 | >167 | 0.59 |
| ASO-798 | mX 6 LNA 2 16 | 1.85 | 0.02 | 6.93 | >167 | 0.86 |
| ASO-799 | mX 6 LNA 2 17 | 1.05 | 0.44 | 7.91 | >167 | 0.59 |
| ASO-800 | mX 6 LNA 2 18 | 1.68 | 0.94 | 4.10 | >167 | 0.49 |
| ASO-795 | mX 6 LNA 2 3 | 0.60 | 0.42 | 7.57 | >167 | 0.63 |
| ASO-801 | mX 6 LNA 2 19 | 0.86 | 0.29 | 8.92 | >167 | 0.65 |
| ASO-802 | mX 6 LNA 2 20 | 1.85 | 0.83 | 7.90 | >167 | 0.65 |
| ASO-803 | mX 6 LNA 3 4 | 0.74 | 0.01 | 9.68 | >167 | 0.84 |
| ASO-804 | mX 6 LNA 3 5 | 0.44 | 0 | 8.62 | >167 | 0.85 |
| ASO-805 | mX 6 LNA 3 16 | 0.68 | 0.1 | 9.68 | >167 | 0.96 |
| ASO-806 | mX 6 LNA 3 17 | 0.58 | 0.01 | 17.86 | >167 | 0.63 |
| ASO-807 | mX 6 LNA 3 18 | 0.94 | 0.21 | 12.89 | >167 | 0.62 |
| ASO-808 | mX 6 LNA 3 19 | 0.99 | 0.61 | 6.57 | >167 | 0.75 |
| ASO-809 | mX 6LNA 3 20 | 1.27 | 0.08 | 7.999 | >167 | 0.64 |
| ASO-810 | mX 6 LNA 4 5 | 0.35 | 0.00 | 7.53 | >167 | 0.90 |
| ASO-811 | mX 6 LNA 4 16 | 0.85 | 0.27 | 6.58 | >167 | 0.95 |
| ASO-812 | mX 6 LNA 4 17 | 0.77 | 0.05 | 16.40 | >167 | 0.67 |
| ASO-813 | mX 6 LNA 4 18 | 1.16 | 0.24 | 9.20 | >167 | 0.74 |
| ASO-814 | mX 6 LNA 4 19 | 0.72 | 0.01 | 6.18 | >167 | 0.79 |
| ASO-815 | mX 6 LNA 4 20 | 0.98 | 0.06 | 7.72 | >167 | 0.77 |
| ASO-816 | mX 6 LNA 5 16 | 0.85 | 0.21 | 8.83 | >167 | 1.15 |
| ASO-817 | mX 6 LNA 5 17 | 0.15 | 0.001 | 12.24 | >167 | 0.79 |
| ASO-818 | mX 6 LNA 5 18 | 0.24 | 0.01 | 13.92 | >167 | 0.81 |
| ASO-819 | mX 6 LNA 5 19 | 0.13 | 0.12 | 17.46 | >167 | 0.92 |
| ASO-820 | mX 6 LNA 5 20 | 0.69 | 0.195 | 37.08 | >167 | 0.92 |
| ASO-821 | mX 6 LNA 16 17 | 0.436 | 0.188 | 47.92 | >167 | 0.91 |
| ASO-822 | mX 6 LNA 16 18 | 0.46 | 0 | 14.15 | >167 | 0.90 |
| ASO-823 | mX 6 LNA 16 19 | 0.86 | 0.12 | 21.64 | >167 | 1.02 |
| ASO-824 | mX 6 LNA 16 20 | 1.21 | 0.55 | 20.45 | >167 | 0.96 |
| ASO-825 | mX 6 LNA 17 18 | 1.07 | 0.45 | 14.91 | >167 | 0.64 |
| ASO-826 | mX 6 LNA 17 19 | 1.48 | 0.68 | 29.46 | >167 | 0.69 |
| ASO-827 | mX 6 LNA 17 20 | 2.16 | 1.48 | 24.31 | >167 | 0.72 |
| ASO-828 | mX 6 LNA 18 19 | 2.17 | 1.74 | 14.91 | >167 | 0.92 |
| ASO-829 | mX 6 LNA 18 20 | 1.76 | 1.41 | 16.49 | >167 | 0.70 |
| ASO-830 | mX 6 LNA 19 20 | 1.45 | 0.73 | 27.04 | >167 | 0.87 |
| ASO-672 | mX 6 | 0.52 | 0.17 | 25.53 | >167 | 0.95 |
| ASO-1 | | 1 | 1 | 10.46 | >167 | 1 |

### Example 33: LNA Pattern Screen on ASO-677

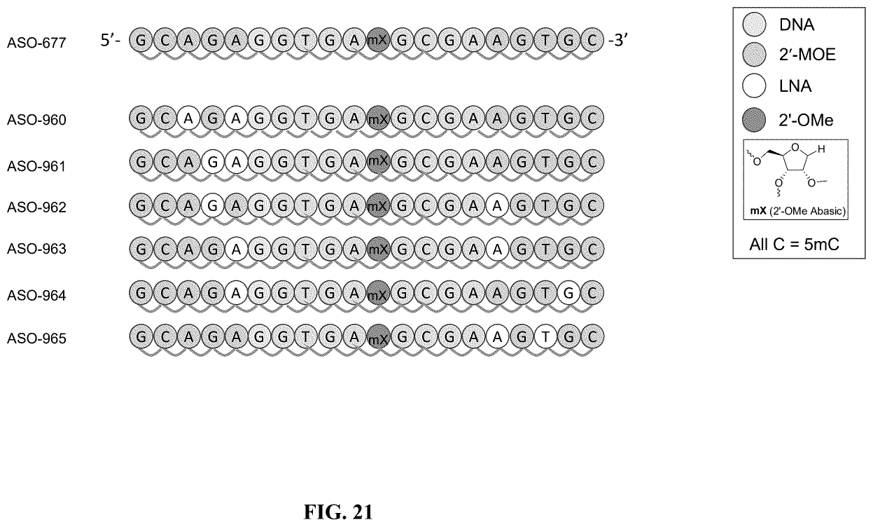

A pattern screen was performed to identify additional LNA modification to ASO-677 and ASO-960, ASO-961, ASO-962, ASO-963, ASO-964, and ASO-965, were prepared (FIG. 21). The ASOs were subjected to the assays described in Example 2 to determine RNase H-mediated degradation, cytotoxicity, TLR8 pathway activation, and caspase activation as compared to control ASO-1 and ASO-677 (Table 20). ASO-962, ASO-963, ASO-964, and ASO-965, improved RNase H activity from ASO-677 and maintained lower Caspase activity as compared to ASO-1 and similar hTLR8 activity as ASO-1.

TABLE 20
Assay Results for ASOs based on ASO-677 with different LNA patterns

| LNA | | Caspase | Caspase | | | |
|---|---|---|---|---|---|---|
| Patterns | | Fold over | Fold over | | | hTLR8 |
| on | | ASO-1 | ASO-1 | EC50 | CC50 | Fold over |
| ASO-677 | Position | 167 nM | 56 nM | nM | nM | ASO-1 |
| ASO-960 | X + LNA 3 and 5 | 0.86 | 0.83 | 4.40 | 90.48 | 1.14 |
| ASO-961 | X + LNA 4 and 5 | 1.19 | 1.36 | 5.06 | >167 | 1.25 |
| ASO-962 | X + LNA 4 and 16 | 0.52 | 0.17 | 7.0 | >167 | 1.01 |
| ASO-963 | X + LNA 5 and 16 | 0.55 | 0.24 | 7.86 | >167 | 0.94 |
| ASO-964 | X + LNA 5 and 19 | 0.87 | 0.84 | 8.09 | >167 | 0.81 |
| ASO-965 | X + LNA 16 and 18 | 0.61 | 0.36 | 6.74 | >167 | 0.90 |
| ASO-677 | mX 11-(2′ OMe) | 0.44 | 0.06 | 23.03 | >167 | 1.07 |
| ASO-1 | | 1 | 1 | 7.76 | >167 | 1 |

### Example 34: Mono-GalNAc Modifications to LNA and 2′OMe Abasic Monomer Modified ASO's

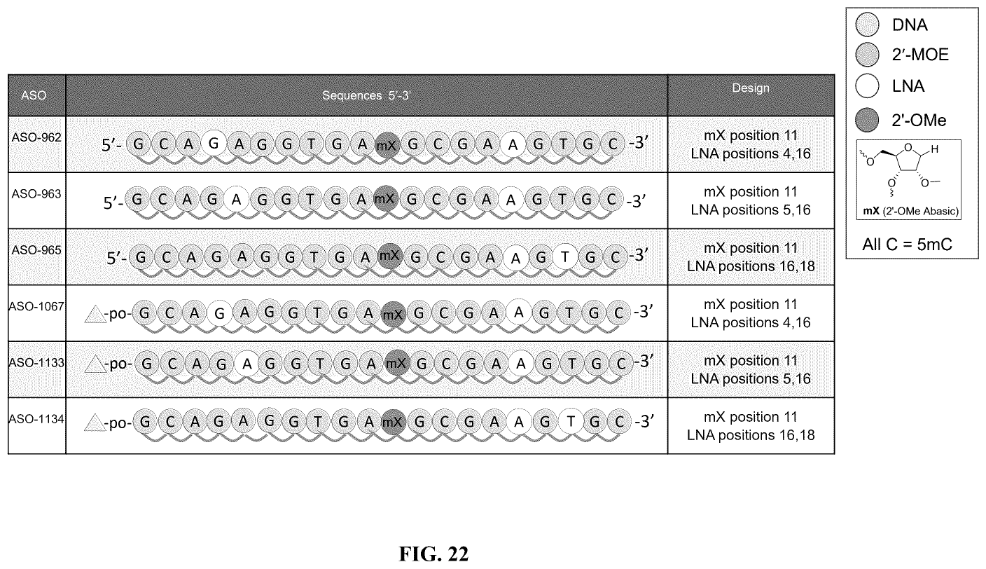

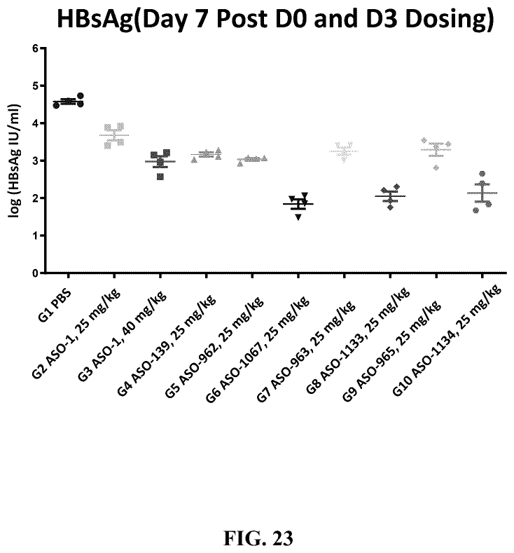

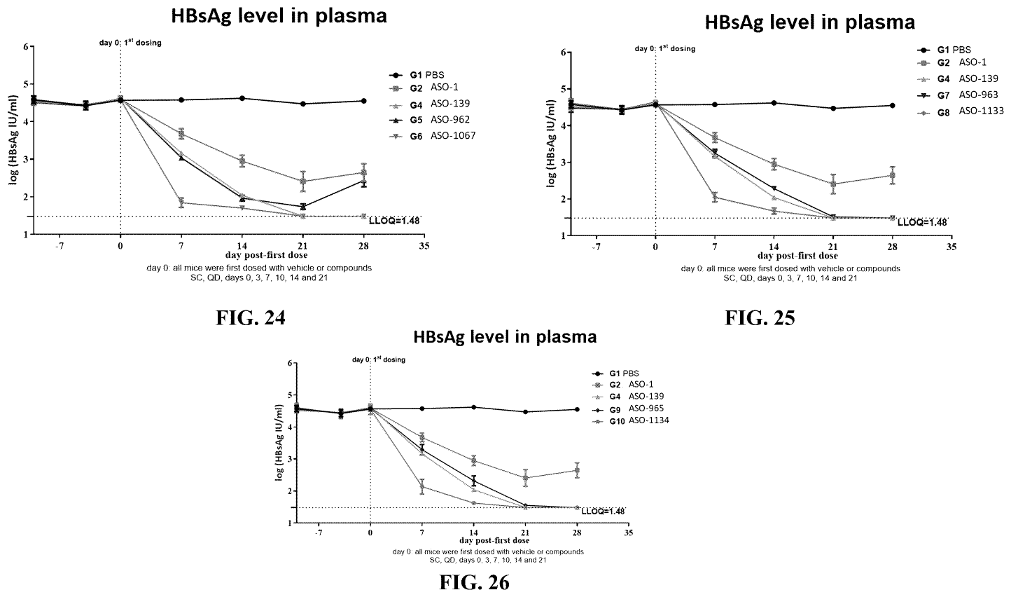

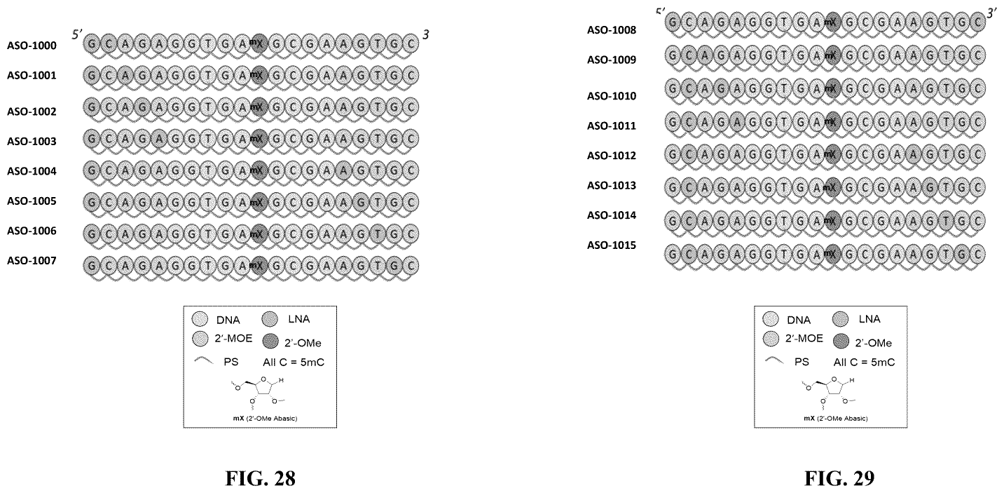

The effect of 5′ mono GalNAc modifications to ASOs with LNA and 2′OMe abasic monomer modifications was assessed. The parent construct ASO-962 was used to produce ASO-1067, the parent strain ASO-963 was used to produce ASO-1133, and the parent strain ASO-965 was used to produce ASO-1134 (FIG. 22). The effect of the produced ASOs on HBsAg levels in plasma were evaluated in an AAV-HBV C57BL/6 mouse model as described in Example 25. Group 1 was injected with PBS, Group 2 with ASO-1 control at 25 mg/kg, Group 3 with ASO-1 at 40 mg/kg control, Group 4 with ASO-139 at 25 kg/mg, Group 5 with ASO-962 at 25 kg/mg, Group 6 with ASO-1067 at 25 kg/mg, Group 7 with ASO-963 at 25 mg/kg, Group 8 with ASO-1133 at 25 mg/kg, Group 9 with ASO-965 at 25 mg/kg, and Group 10 with ASO-1134 at 25 mg/kg. Each group received 6 subcutaneous doses on days 0, 3, 7, 10, 14 and 21. On day 7, HBsAg was measured day 7 post Day 0 and D3 treatments, As shown in FIG. 28, 5′ Mono GalNAc ASOs were significantly more potent than unconjugated ASOs of same sequences, and two reference sequences on Day 7. When full time course of the study was analyzed (FIGS. 24-26), three 2′ OMe Abasic monomer modified, LNA containing ASOs ASO-962, ASO-963 and ASO-965 showed better in vivo potency than ASO-1, similar potency as ASO-139. The 5′ Mono GalNAc modified ASO-1067, ASO-1133 and ASO-1134 are significantly more potent than ASO-1, ASO-139 and their respective unconjugated sequences: ASO-962, ASO-963 and ASO-965.

Additionally, in vitro profiles—including caspase activity, hTLR8 activity, EC50 and CC50—were determined for mono GalNAc versions of several ASOs. As shown in the following tables (Tables 21-23), hTLR8 activity was similar between conjugated and unconjugated ASOs, RNaseH activity was generally reduced in vitro for ASOs conjugated to GalNAc, and mono GalNAc generally improved caspase 3/7 activity compared to unconjugated ASOs.

TABLE 21
In vitro Profiles of Mono GalNAc Versions of Reference ASOs

| | | Caspase | Caspase | | | |
|---|---|---|---|---|---|---|
| | | Fold over | Fold over | hTLR8 | | |
| | | ASO-1 | ASO-1 | Activity Over | HepG2.2.15 | HepG2.2.15 |
| Compound ID | Design | 167 nM | 56 nM | ASO-1 | EC50 (nM) | CC50 (nM) |
| ASO-1 | | 1 | 1 | 1.00 | 3.37 | >167 |
| ASO-1057 | ASO-1 | 0.95 | 0.48 | 0.88 | 10.15 | >167 |
| | GalNAc | | | | | |
| ASO-139 | | 1.02 | 0.92 | 0.78 | 0.58 | >167 |
| ASO-1058 | ASO-139 | 1.51 | 2.15 | 0.84 | 2.21 | >167 |
| | GalNAc | | | | | |

TABLE 22
In vitro Profiles of Mono GalNAc and Non-GalNAc Versions of ASOs

| | | Caspase | Caspase | | | hTLR8 |
|---|---|---|---|---|---|---|
| | | Fold over | Fold over | | | Fold |
| | | ASO-1 | ASO-1 | | | over |
| Compound ID | Design | 167 nM | 56 nM | EC50 (nM) | CC50 (nM) | ASO-1 |
| ASO-965 | ASO-6 w LNA | 1.51 | 1.27 | 3.70 | >167 | 1.32 |
| | Pos-15_17 | | | | | |
| ASO-1134 | mono GalNAc | 0.93 | 0.27 | 10.88 | >167 | 1.23 |
| | ASO-965 | | | | | |
| ASO-6 | | 1.19 | 0.46 | 9.51 | >167 | 1.12 |
| ASO-651 | mono GalNAc | 0.27 | 0.11 | 19.84 | >167 | 1.11 |
| | ASO-6 | | | | | |
| ASO-962 | ASO-677 LNA 4 | 0.68 | 0.56 | 12.59 | >167 | 0.92 |
| | and 16 | | | | | |
| ASO-1067 | mono GalNAc-ASO- | 0.45 | 0.17 | 17.36 | >167 | 0.96 |
| | 962 | | | | | |
| ASO-963 | ASO-677 LNA 5 and | 0.79 | 0.74 | 9.98 | >167 | 0.85 |
| | 16 | | | | | |
| ASO-1133 | mono GalNAc-ASO- | 0.43 | 0.19 | 17.71 | >167 | 1.26 |
| | 963 | | | | | |
| ASO-1 | | 1 | 1 | 4.82 | >167 | 1.00 |

TABLE 23
In vitro Profiles of Mono GalNAc and Non-GalNAc Versions of ASOs

| | | Caspase | Caspase | | | hTLR8 |
|---|---|---|---|---|---|---|
| | | Fold over | Fold over | | | Fold |
| | | ASO-1 | ASO-1 | | | over |
| Compound ID | Design | 167 nM | 56 nM | EC50 (nM) | CC50 (nM) | ASO-1 |
| ASO-965 | ASO-677 LNA 16 | 0.77 | 0.36 | 9.41 | >167 | 1.11 |
| | and 18 | | | | | |
| ASO-1134 | 5′ mono GalNAc- | 0.75 | 0.09 | 9.79 | >167 | 1.18 |
| | ASO-965 | | | | | |
| ASO-687 | ASO- | 0.86 | 0.34 | 7.53 | >167 | 1.03 |
| | 1_MMPos10_dT | | | | | |
| ASO-1195 | 5′ mono GalNAc- | 0.45 | 0.14 | 15.44 | >167 | 0.93 |
| | ASO-687 | | | | | |
| ASO-677 | ASO-1_mX 11 | 0.65 | 0.53 | 11.19 | >167 | 0.91 |
| ASO-1166 | 5′ mono GalNAc | 0.25 | 0.13 | 24.23 | >167 | 0.94 |
| | ASO-677 | | | | | |
| ASO-1020 | ASO-1 mX 11 LNA | 0.64 | 0.42 | 5.81 | >167 | 0.94 |
| | 3 18 | | | | | |
| ASO-1194 | 5′ mono GalNAc | 0.65 | 0.16 | 8.11 | >167 | 0.83 |
| | ASO-1020 | | | | | |
| ASO-1036 | ASO-1 mX 11 LNA | 0.77 | 0.51 | 3.65 | >167 | 0.81 |
| | 18 19 | | | | | |
| ASO-1181 | mono GalNAc ASO- | 0.71 | 0.21 | 9.97 | >167 | 0.75 |
| | 1036 | | | | | |
| ASO-1037 | ASO-1 mX 11 LNA | 10.78 | 0.52 | 4.61 | >167 | 0.70 |
| | 18 20 | | | | | |
| ASO-1179 | mono GalNAc ASO- | 0.78 | 0.23 | 6.73 | >167 | 0.92 |
| | 1037 | | | | | |
| ASO-945 | Mismatched ASO- | 0.91 | 0.45 | 6.55 | >167 | 0.98 |
| | 693_LNA-16_18 | | | | | |
| ASO-1180 | mono GalNAc ASO- | 0.50 | 0.21 | 9.07 | >167 | 1.20 |
| | 945 | | | | | |
| ASO-1 | | 1 | 1 | 4.82 | >167 | 1.00 |

### Example 35: LNA Modification Pattern Screen on Mismatched ASO-686, ASO-692, ASO-693, ASO-683, ASO-690

A LNA modification pattern screen was performed on the mismatched ASO-686 to produce ASO-966, ASO-967, ASO-968, ASO-969, ASO-970, and ASO-971. The LNA pattern identified in Example 35 was applied to ASO-692 to produce ASO-934, ASO-935, ASO-936, ASO-937, ASO-938, and ASO-939; ASO-693 to produce ASO-940, ASO-941, ASO-942, ASO-943, ASO-944, and ASO-945; ASO-683 to produce ASO-972, ASO-973, ASO-974, ASO-975, ASO-976, and ASO-977; and ASO-690 to produce ASO-978, ASO-979, ASO-980, ASO-981, ASO-982, and ASO-983. The ASO's were subjected to the assays described in Example 2 to determine RNAse H-mediated degradation, cytotoxicity, TLR8 pathway activation, and caspase activation as compared to control ASO-1 and ASO-686 (Table 24). ASO-686 with LNA modification increased toxicity as compared to the parent construct or ASO-1. ASO-939 showed an improvement in caspase over ASO-1 (Table 25). ASO-945 improved caspase and EC50 as compared to ASO-1 (Table 26). ASO-977, ASO-980 and ASO-982 retained lower caspase activity (Table 27 and Table 28).

TABLE 24
Assay Results for ASO's based on ASO-686 with different LNA patterns

| LNA | | Caspase | Caspase | | | |
|---|---|---|---|---|---|---|
| Patterns | | Fold over | Fold over | | | TLR8 |
| on | | ASO-1 | ASO-1 | EC50 | CC50 | Fold over |
| ASO-686 | Position | 167 nM | 56 nM | nM | nM | ASO-1 |
| ASO-966 | G + LNA 3 and 5 | 2.92 | 4.33 | 4.12 | 89.5 | 0.74 |
| ASO-967 | G + LNA 4 and 5 | 2.92 | 4.69 | 3.13 | 63.35 | 0.81 |
| ASO-968 | G + LNA 4 and 16 | 2.16 | 1.93 | 4.26 | >167 | 0.86 |
| ASO-969 | G + LNA 5 and 16 | 2.52 | 4.64 | 5.64 | >167 | 0.85 |
| ASO-970 | G + LNA 5 and 19 | 2.64 | 4.55 | 5.79 | 166.83 | 0.74 |
| ASO-971 | G + LNA 16 and 18 | 1.89 | 1.54 | 3.66 | >167 | 1.07 |
| ASO-686 | G10-2′-Deoxy MM) | 1.01 | 0.23 | 16.68 | >167 | 1.09 |
| ASO-1 | | 1 | 1 | 7.76 | >167 | 1 |

TABLE 25
Assay Results for ASO's based on ASO-692 with different LNA patterns

| LNA | | Caspase | Caspase | | | |
|---|---|---|---|---|---|---|
| Patterns | | Fold over | Fold over | | | TLR8 |
| on ASO- | | ASO-1 | ASO-1 | EC50 | CC50 | Fold over |
| 692 | Position | 167 nM | 56 nM | nM | nM | ASO-1 |
| ASO-934 | LNA 3 and 5 | 1.91 | 3.51 | 4.82 | >167 | 0.91 |
| ASO-935 | LNA 4 and 5 | 2.30 | 4.53 | 10.98 | 64.26 | 1.04 |
| ASO-936 | LNA 4 and 16 | 1.20 | 1.49 | 7.74 | >167 | 1.10 |
| ASO-937 | LNA 5 and 16 | 1.31 | 2.63 | 3.40 | >167 | 0.90 |
| ASO-938 | LNA 5 and 19 | 1.49 | 2.84 | 21.15 | >167 | 0.74 |
| ASO-939 | LNA 16 and 18 | 0.89 | 0.86 | 8.26 | >167 | 0.87 |
| ASO-139 | | 0.20 | 0.27 | 13.18 | >167 | 0.78 |
| ASO-1 | | 1 | 1 | 7.76 | >167 | 1 |

TABLE 26
Assay Results for ASO's based on ASO-693 with different LNA patterns

| LNA | | Caspase | Caspase | | | |
|---|---|---|---|---|---|---|
| Patterns | | Fold over | Fold over | | | TLR8 |
| on ASO- | | ASO-1 | ASO-1 | EC50 | CC50 | Fold over |
| 693 | Position | 167 nM | 56 nM | nM | nM | ASO-1 |
| ASO-940 | LNA 3 and 5 | 2.14 | 2.25 | 7.23 | >167 | 0.71 |
| ASO-941 | LNA 4 and 5 | 2.08 | 2.17 | 5.97 | >167 | 0.77 |
| ASO-942 | LNA 4 and 16 | 0.95 | 0.65 | 5.37 | >167 | 0.88 |
| ASO-943 | LNA 5 and 16 | 1.62 | 1.46 | 6.46 | >167 | 1.07 |
| ASO-944 | LNA 5 and 19 | 1.69 | 1.10 | 8.88 | >167 | 0.84 |
| ASO-945 | LNA 16 and 18 | 0.55 | 0.26 | 3.75 | >167 | 1.10 |
| ASO-693 | 2′-Deoxy MM | 0.16 | 0.10 | 27.78 | >167 | 0.78 |
| ASO-1 | | 1 | 1 | 7.76 | >167 | 1 |

TABLE 27
Assay Results for ASO's based on ASO-683 with different LNA patterns

| LNA | | Caspase | Caspase | | | |
|---|---|---|---|---|---|---|
| Patterns | | Fold over | Fold over | | | TLR8 |
| on ASO- | | ASO-1 | ASO-1 | EC50 | CC50 | Fold over |
| 683 | Position | 167 nM | 56 nM | nM | nM | ASO-1 |
| ASO-972 | LNA 3 and 5 | 1.64 | 1.34 | 4.28 | >167 | 1.23 |
| ASO-973 | LNA 4 and 5 | 1.76 | 1.20 | 4.01 | >167 | 1.57 |
| ASO-974 | LNA 4 and 16 | 1.07 | 0.35 | 7.16 | >167 | 1.67 |
| ASO-975 | LNA 5 and 16 | 1.27 | 0.55 | 5.53 | >167 | 1.42 |
| ASO-976 | LNA 5 and 19 | 1.24 | 0.78 | 5.14 | >167 | 1.14 |
| ASO-977 | LNA 16 and 18 | 0.50 | 0.16 | 6.04 | >167 | 1.31 |
| ASO-683 | 2′-Deoxy MM | 0.27 | 0 | 12.2 | >167 | 0.82 |
| ASO-1 | | 1 | 1 | 4.95 | >167 | 1 |

TABLE 28
Assay Results for ASO's based on ASO-690 with different LNA patterns

| LNA | | Caspase | Caspase | | | |
|---|---|---|---|---|---|---|
| Patterns | | Fold over | Fold over | | | hTLR8 |
| on | | ASO-1 | ASO-1 | EC50 | CC50 | Fold Over |
| ASO-690 | Position | 167 nM | 56 nM | nM | nM | ASO-1 |
| ASO-978 | LNA 3 and 5 | 1.14 | 1.12 | 5.00 | >167 | 1.03 |
| ASO-979 | LNA 4 and 5 | 1.36 | 1.00 | 3.65 | >167 | 0.75 |
| ASO-980 | LNA 4 and 16 | 0.55 | 0.24 | 4.05 | >167 | 0.86 |
| ASO-981 | LNA 5 and 16 | 1.03 | 0.48 | 4.76 | >167 | 0.80 |
| ASO-982 | LNA 5 and 19 | 0.63 | 0.71 | 3.98 | >167 | 0.71 |
| ASO-983 | LNA 16 and 18 | 0.54 | 0.91 | 2.79 | 57.801 | 0.74 |
| ASO-690 | 2′-Deoxy MM | 0.64 | 0.56 | 7.24 | >167 | 1.15 |
| ASO-1 | | 1 | 1 | 4.95 | >167 | 1 |

### Example 36: LNA Modification Pattern to 2′ Deoxy Abasic ASO Parental Sequences, ASO-707 and ASO-706

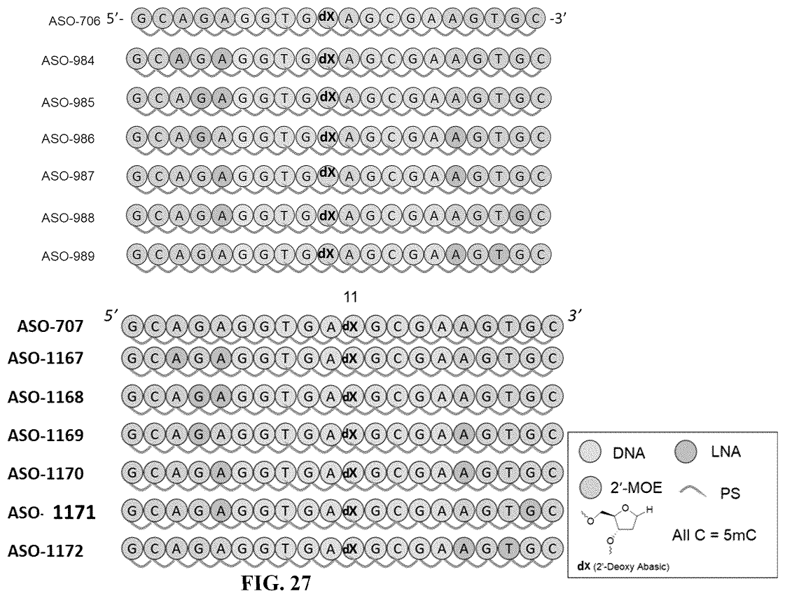

The LNA pattern identified in Example 35 was applied to parental ASO-706 to produce ASO-984, ASO-985, ASO-986, ASO-987, ASO-988, and ASO-989; and to parental 707 to produce ASO 1167, ASO 1168, ASO 1169, ASO 1170, ASO 1171 and ASO1172 (FIG. 27). The ASO's were subjected to the assays described in Example 2 to determine RNAse H-mediated degradation, cytotoxicity, TLR8 pathway activation, and caspase activation. ASO-985, ASO-989 and ASO-988 improved overall profile over parent ASO-706. ASO-1167, ASO-1168 and ASO-1171 showed overall improvement over parent ASO-707. (Table 29)

TABLE 29
Assay Results for ASO's based on ASO-706
and ASO-707 with different LNA patterns

| | | Caspase | Caspase | | | |
|---|---|---|---|---|---|---|
| LNA | | Fold over | Fold over | | | hTLR8 |
| Pattern on | | ASO-1 | ASO-1 | EC50 | CC50 | Fold over |
| ASO-706 | Position | 167 nM | 56 nM | nM | nM | ASO-1 |
| ASO-984 | LNA 3 and 5 | 0.63 | 0.38 | 5.43 | >167 | 0.73 |
| ASO-985 | LNA 4 and 5 | 0.62 | 0.25 | 5.51 | >167 | 0.82 |
| ASO-986 | LNA 4 and 16 | 0.43 | 0.23 | 26.30 | >167 | 0.90 |
| ASO-987 | LNA 5 and 16 | 0.52 | 0.09 | 10.67 | >167 | 0.82 |
| ASO-988 | LNA 5 and 19 | 0.47 | 0.59 | 6.55 | >167 | 0.85 |
| ASO-989 | LNA 16 and 18 | 0.26 | 0.54 | 12.99 | >167 | 1.11 |
| ASO-706 | 2′-Deoxy Abasic | 0.71 | 0.40 | 22.85 | >167 | 0.89 |
| ASO-1 | | 1 | 1 | 4.95 | >167 | 1 |
| | | Caspase | Caspase | | | |
| LNA | | Fold over | Fold over | | | hTLR8 |
| Pattern on | | ASO-1 | ASO-1 | EC50 | CC50 | Fold over |
| ASO-707 | Position | 167 nM | 56 nM | nM | nM | ASO-1 |
| ASO-1167 | LNA 3 and 5 | 0.73 | 0.56 | 2.24 | >167 | 1.07 |
| ASO-1168 | LNA 4 and 5 | 0.83 | 0.81 | 5.75 | >167 | 1.09 |
| ASO-1169 | LNA 4 and 16 | 0.73 | 0.67 | 7.88 | >167 | 1.11 |
| ASO-1170 | LNA 5 and 16 | 0.64 | 0.82 | 6.93 | >167 | 1.05 |
| ASO-1171 | LNA 5 and 19 | 0.63 | 0.75 | 4.56 | >167 | 0.93 |
| ASO-1172 | LNA 16 and 18 | 0.30 | 0.00 | 7.36 | >167 | 1.08 |
| ASO-707 | 2′-Deoxy Abasic | 0.78 | 0.88 | 11.67 | >167 | 1.03 |
| ASO-1 | | 1 | 1 | 4.02 | >167 | 1.00 |

### Example 37: Single LNA Modification on Mismatch ASO's

ASOs were prepared with an LNA modification at positions 5 or 17 and a mismatch at positions 9, 10, 11, or 12. ASO-683 was used to prepare ASO-947 and ASO-946, ASO-686 was used to prepare ASO-948 and ASO-949, ASO-690 was used to prepare ASO-950 and ASO-951 (Table 1), ASO-692 was used to prepare ASO-952 and ASO-953, and ASO-693 was used to prepare ASO-954 and ASO-955. The ASOs were subjected to the assays described in Example 2 to determine RNAse H-mediated degradation, cytotoxicity, TLR8 pathway activation, and caspase activation (Table 30 and Table 31). ASO-947 showed lower caspase activity compared to ASO-1 (Table 30).

TABLE 30
Assay Results for ASOs different LNA and Mismatch patterns

| | | Caspase | Caspase | |
|---|---|---|---|---|
| | | Fold over | Fold over | TLR8 |
| | Position (LNA | ASO-1 | ASO-1 | Fold over |
| ASO | pattern and MM) | 167 nM | 56 nM | ASO-1 |
| ASO-946 | LNA 5 MM9 | 1.41 | 1.03 | 1.24 |
| ASO-947 | LNA 17 MM9 | 0.36 | 0.71 | 1.36 |
| ASO-948 | LNA 5 MM10 | 1.89 | 2.80 | 1.00 |
| ASO-949 | LNA 17 MM10 | 2.18 | 1.41 | 0.94 |
| ASO-950 | LNA 5 MM11 | 1.69 | 1.14 | 0.85 |
| ASO-951 | LNA 17 MM11 | 1.52 | 0.79 | 0.89 |
| ASO-139 | LNA17, 19, 20 | 1.48 | 2.16 | 0.72 |
| ASO-1 | | 1 | 1 | 1 |

TABLE 31
Assay Results for ASOs different LNA and Mismatch patterns

| | | Caspase | Caspase | |
|---|---|---|---|---|
| LNA | | Fold over | Fold over | TLR8 |
| Pattern + | | ASO-1 | ASO-1 | Fold over |
| MM | Position | 167 nM | 56 nM | ASO-1 |
| ASO-952 | LNA 5 MM12 | 0.57 | 1.96 | 0.62 |
| ASO-953 | LNA 17 MM12 | 1.17 | 0.46 | 0.61 |
| ASO-954 | LNA 5 MM12 | 1.23 | 0.82 | 0.75 |
| ASO-955 | LNA 17 MM12 | 0.91 | 1.03 | 0.73 |
| ASO-139 | LNA17, 19, 20 | 1.48 | 2.16 | 0.72 |
| ASO-1 | | 1 | 1 | 1 |

### Example 38: LNA Modification and 2′-OMe Abasic ASOs

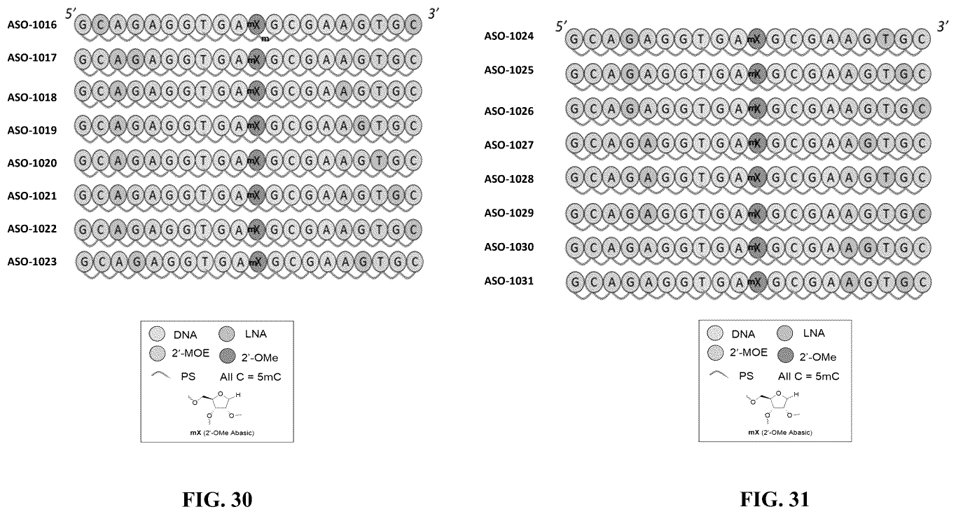

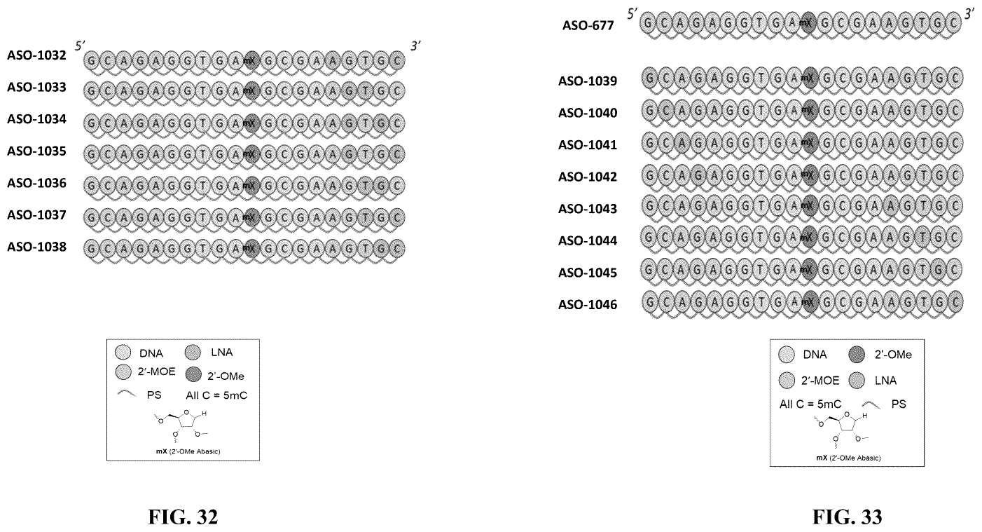

ASO-677 from Example 28 that contains a 2′-OMe Abasic modification at position 11 was used to create additional ASOs using a 2×LNA walk as shown in FIG. 28, FIG. 29, FIG. 30, FIG. 31, and FIG. 32, and 1×LNA walk as shown in FIG. 33. The ASOs were subjected to the assays described in Example 2 to determine RNAse H-mediated degradation, cytotoxicity, TLR8 pathway activation, and caspase activation, with 2×LNA walk showing better improvement than 1×LNA walk (Table 32, Table 33, Table 34, Table 35, Table 36, and Table 37).

TABLE 32
Assay Results for ASOs based on ASO-677 different
LNA and 2′-OMe abasic monomer patterns

| | | Caspase | Caspase | | | |
|---|---|---|---|---|---|---|
| ASO-677 | | Fold over | Fold over | | | TLR8 |
| 2x LNA | | ASO-1 | ASO-1 | EC50 | CC50 | Fold over |
| walk | Position | 167 nM | 56 nM | nM | nM | ASO-1 |
| ASO-1000 | mX 11 LNA 1 2 | 0.75 | 0.26 | 2.99 | >167 | 0.89 |
| ASO-1001 | mX 11 LNA 1 3 | 0.85 | 0.34 | 5.64 | >167 | 0.87 |
| ASO-1002 | mX 11 LNA 1 4 | 0.98 | 0.18 | 5.12 | >167 | 0.88 |
| ASO-1003 | mX 11 LNA 1 5 | 0.98 | 0.25 | 3.91 | >167 | 0.85 |
| ASO-1004 | mX 11 LNA 1 16 | 0.55 | 0.17 | 5.71 | >167 | 0.69 |
| ASO-1005 | mX 11 LNA 1 17 | 0.81 | 0.34 | 5.72 | >167 | 0.71 |
| ASO-1006 | mX 11 LNA 1 18 | 0.77 | 0.41 | 3.78 | >167 | 0.68 |
| ASO-1007 | mX 11 LNA 1 19 | 0.82 | 0.44 | 6.16 | >167 | 0.69 |
| ASO-677 | mX 11 | 0.79 | 0.37 | 7.83 | >167 | 1.04 |
| ASO-1 | | 1 | 1 | 4.29 | >167 | 1 |

TABLE 33
Assay Results for ASOs based on ASO-677
different LNA and 2′-OMe Abasic patterns

| | | Caspase | Caspase | | | |
|---|---|---|---|---|---|---|
| ASO-677 | | Fold over | Fold over | | | TLR8 |
| 2x LNA | | ASO-1 | ASO-1 | EC50 | CC50 | Fold over |
| walk | Position | 167 nM | 56 nM | nM | nM | ASO-1 |
| ASO-1008 | mX 11 LNA 1 20 | 0.82 | 0.35 | 4.68 | >167 | 0.74 |
| ASO-1009 | mX 11 LNA 2 3 | 1.10 | 0.55 | 3.26 | >167 | 0.98 |
| ASO-1010 | mX 11 LNA 2 4 | 1.29 | 0.80 | 3.52 | >167 | 0.96 |
| ASO-1011 | mX 11 LNA 2 5 | 1.26 | 0.70 | 3.18 | >167 | 0.94 |
| ASO-1012 | mX 11 LNA 2 16 | 0.84 | 0.46 | 4.82 | >167 | 1.16 |
| ASO-1013 | mX 11 LNA 2 17 | 0.95 | 0.63 | 4.22 | >167 | 1.16 |
| ASO-1014 | mX 11 LNA 2 18 | 1.03 | 0.36 | 2.97 | >167 | 0.91 |
| ASO-1015 | mX 11 LNA 2 19 | 1.06 | 0.72 | 4.61 | >167 | 0.94 |
| ASO-677 | mX 11 | 0.79 | 0.37 | 7.83 | >167 | 1.04 |
| ASO-1 | | 1 | 1 | 4.29 | >167 | 1 |

TABLE 34
Assay Results for ASOs based on ASO-677 different
LNA and 2′-OMe abasic monomer patterns

| | | Caspase | Caspase | | | |
|---|---|---|---|---|---|---|
| ASO-677 | | Fold over | Fold over | | | TLR8 |
| 2x LNA | | ASO-1 | ASO-1 | EC50 | CC50 | Fold over |
| walk | Position | 167 nM | 56 nM | nM | nM | ASO-1 |
| ASO-1016 | mX 11 LNA 2 20 | 1.08 | 0.54 | 2.69 | >167 | 0.96 |
| ASO-1017 | mX 11 LNA 3 4 | 1.05 | 0.26 | 3.89 | >167 | 1.29 |
| ASO-1018 | mX 11 LNA 3 16 | 0.94 | 0.42 | 4.32 | >167 | 1.18 |
| ASO-1019 | mX 11 LNA 3 17 | 1.03 | 0.36 | 3.87 | >167 | 0.88 |
| ASO-1020 | mX 11 LNA 3 18 | 0.77 | 0.28 | 1.97 | >167 | 0.97 |
| ASO-1021 | mX 11 LNA 3 19 | 0.80 | 0.45 | 2.26 | >167 | 0.82 |
| ASO-1022 | mX 11 LNA 3 20 | 0.88 | 0.60 | 2.68 | >167 | 0.93 |
| ASO-1023 | mX 11 LNA 4 17 | 0.92 | 0.34 | 4.36 | >167 | 1.14 |
| ASO-677 | mX 11 | 0.79 | 0.37 | 7.83 | >167 | 1.04 |
| ASO-1 | | 1 | 1 | 4.29 | >167 | 1 |

TABLE 35
Assay Results for ASOs based on ASO-677 different
LNA and 2′-OMe abasic monomer patterns

| | | Caspase | Caspase | | | |
|---|---|---|---|---|---|---|
| ASO-677 | | Fold over | Fold over | | | TLR8 |
| 2x LNA | | ASO-1 | ASO-1 | EC50 | CC50 | Fold over |
| walk | Position | 167 nM | 56 nM | nM | nM | ASO-1 |
| ASO-1024 | mX 11 LNA 4 18 | 0.95 | 0.39 | 1.57 | >167 | 1.13 |
| ASO-1025 | mX 11 LNA 4 19 | 0.99 | 0.38 | 4.14 | >167 | 1.11 |
| ASO-1026 | mX 11 LNA 4 20 | 1.05 | 0.39 | 3.44 | >167 | 1.13 |
| ASO-1027 | mX 11 LNA 5 17 | 1.11 | 0.48 | 4.17 | >167 | 1.02 |
| ASO-1028 | mX 11 LNA 5 18 | 0.83 | 0.52 | 2.46 | >167 | 1.15 |
| ASO-1029 | mX 11 LNA 5 20 | 0.91 | 0.76 | 2.48 | >167 | 1.09 |
| ASO-1030 | mX 11 LNA 16 17 | 0.94 | 0.39 | 7.49 | >167 | 1.21 |
| ASO-1031 | mX 11 LNA 16 19 | 0.50 | 0.30 | 8.19 | >167 | 1.06 |
| ASO-677 | mX 11 | 0.79 | 0.37 | 7.83 | >167 | 1.04 |
| ASO-1 | | 1 | 1 | 4.29 | >167 | 1 |

TABLE 36
Assay Results for ASOs based on ASO-677 different
LNA and 2′-OMe abasic monomer patterns

| | | Caspase | Caspase | | | |
|---|---|---|---|---|---|---|
| ASO-677 | | Fold over | Fold over | | | TLR8 |
| 2x LNA | | ASO-1 | ASO-1 | EC50 | CC50 | Fold over |
| walk | Position | 167 nM | 56 nM | nM | nM | ASO-1 |
| ASO-1032 | mX 11 LNA 16 20 | 0.86 | 0.41 | 4.84 | >167 | 1.10 |
| ASO-1033 | mX 11 LNA 17 18 | 0.91 | 0.31 | 3.73 | >167 | 1.07 |
| ASO-1034 | mX 11 LNA 17 19 | 0.94 | 0.46 | 4.79 | >167 | 0.98 |
| ASO-1035 | mX 11 LNA 17 20 | 0.97 | 0.65 | 3.92 | >167 | 1.08 |
| ASO-1036 | mX 11 LNA 18 19 | 0.65 | 0.40 | 1.08 | >167 | 1.00 |
| ASO-1037 | mX 11 LNA 18 20 | 0.76 | 0.58 | 2.39 | >167 | 1.07 |
| ASO-1038 | mX 11 LNA 19 20 | 0.81 | 0.82 | 3.78 | >167 | 1.10 |
| ASO-677 | mX 11 | 0.79 | 0.37 | 7.83 | >167 | 1.04 |
| ASO-1 | | 1 | 1 | 4.29 | >167 | 1 |

TABLE 37
Assay Results for ASOs based on ASO-677 different
LNA and 2′-OMe abasic monomer patterns

| | | Caspase | Caspase | | | |
|---|---|---|---|---|---|---|
| ASO-677 | | Fold over | Fold over | | | TLR8 |
| 1x LNA | | ASO-1 | ASO-1 | EC50 | CC50 | Fold over |
| walk | Position | 167 nM | 56 nM | nM | nM | ASO-1 |
| ASO-1039 | mX 11 LNA-Pos1 | 2.26 | 3.89 | 5.93 | >167 | 0.87 |
| ASO-1040 | mX 11 LNA-Pos2 | 2.34 | 3.46 | 5.56 | >167 | 1.16 |
| ASO-1041 | mX 11 LNA-Pos3 | 2.27 | 3.68 | 5.82 | >167 | 1.05 |
| ASO-1042 | mX 11 LNA-Pos4 | 2.15 | 3.21 | 5.52 | >167 | 1.13 |
| ASO-1043 | mX 11 LNA-Pos16 | 1.95 | 4.13 | 8.50 | >167 | 1.21 |
| ASO-1044 | mX 11 LNA-Pos18 | 1.78 | 3.87 | 3.38 | >167 | 0.97 |
| ASO-1045 | mX 11 LNA-Pos19 | 2.10 | 4.36 | 5.50 | >167 | 0.96 |
| ASO-1046 | mX 11 LNA-Pos20 | 2.06 | 4.26 | 5.35 | >167 | 0.99 |
| ASO-677 | mX 11 | 0.74 | 0.48 | 8.07 | >167 | 1.09 |
| ASO-1 | | 1 | 1 | 4.29 | >167 | 1 |

### Example 39: 2′,5′-Dual Modified Amidites in ASO-182

ASO-182 as described in Example 24 was modified. ASO-661 contains a 5cpG modification at position 6 and ASO-662 contains a 5mcpG modification at position 6. ASO-663 contains a 5cpG modification at position 8 and ASO-664 contains a 5mcpG modification at 8. The ASOs were subjected to the assays described in Example 2 to determine RNAse H-mediated degradation, cytotoxicity, TLR8 pathway activation, and caspase activation. Overall there is little improvement on caspase profile (Table 38).

TABLE 38
Assay Results for ASOs based on ASO-182 with 2′,5′-dual modified amidites

| | | Caspase | Caspase | | | |
|---|---|---|---|---|---|---|
| | | Fold over | Fold over | hTLR8 | | |
| | | ASO-1 | ASO-1 | fold over | HepG2.2.25 | HepG2.2.15 |
| ASO | Chem | 167 nM | 56 nM | ASO-1 | EC50 nM | CC50 nM |
| ASO-6 | Ctrl | 1.32 | 2.44 | 1.14 | 7.89 | >167 |
| ASO-182 | 2 LNA 3, 18 | 1.11 | 3.46 | 1.12 | 2.41 | >167 |
| ASO-661 | cpG 6 | 1.14 | 3.37 | 0.82 | 1.76 | >167 |
| ASO-662 | mcpG 6 | 0.80 | 1.74 | 0.85 | 2.50 | >167 |
| ASO-663 | cpG 8 | 0.80 | 1.94 | 0.83 | 2.37 | >167 |
| ASO-664 | mcpG 8 | 0.74 | 2.14 | 0.88 | 1.90 | >167 |
| ASO-1 | Ctrl | 1 | 1 | 1 | 4.46 | >167 |

### Example 40: Screening 21-Mer ASOs with Different Length Gaps and Wings

ASOs were prepared with differing length gap and wing regions. The ASOs were subjected to the assays described in Example 2 to determine RNAse H-mediated degradation, cytotoxicity, TLR8 pathway activation, and caspase activation. ASO-590, ASO-592, ASO-593, ASO-596, ASO-597, ASO-598, ASO-560, and ASO-601 exhibited improved caspase profiles but overall there is significant reduction of hTLR8 agonist activity across every ASO in this group (Table 39).

TABLE 39
Assay Results for 21-mer ASOs with different length Gaps and Wings

| | | Caspase | | | |
|---|---|---|---|---|---|
| | Caspase Fold over | Fold over | | | |
| | ASO-1 | ASO-1 | TLR8 Fold | HepG2.2.15 | HepG2.2.15 |
| ASO | 167 nM | 56 nM | Over ASO-1 | EC50 nM | CC50 nM |
| ASO-6 | 1.35 | 0.91 | 1.23 | 7.89 | >167 |
| ASO-590 | 0.68 | 0.17 | 0.46 | 9.54 | >167 |
| ASO-591 | 0.95 | 0.32 | 0.50 | 8.24 | >167 |
| ASO-592 | 0.71 | 0.18 | 0.49 | 8.18 | >167 |
| ASO-593 | 0.62 | 0.17 | 0.48 | 8.76 | >167 |
| ASO-594 | 1.20 | 0.22 | 0.63 | 6.71 | >167 |
| ASO-595 | 1.45 | 0.73 | 0.54 | 8.93 | >167 |
| ASO-596 | 0.76 | 0.18 | 0.47 | 9.27 | >167 |
| ASO-596 | 0.68 | 0.10 | 0.50 | 8.15 | >167 |
| ASO-597 | 0.78 | 0.17 | 0.52 | 10.59 | >167 |
| ASO-598 | 0.88 | 0.22 | 0.58 | 7.81 | >167 |
| ASO-600 | 0.62 | 0.14 | 0.56 | 10.10 | >167 |
| ASO-601 | 0.55 | 0.09 | 0.56 | 12.34 | >167 |
| ASO-1 | 1 | 1 | 1 | 4.46 | >167 |

### Example 41: MsPA Linker at Differing Positions in ASO-182

ASOs were prepared with a single MsPA linker at differing positions based on ASO-182. The ASOs were subjected to the assays described in Example 2 to determine RNAse H-mediated degradation, cytotoxicity, and TLR8 pathway activation. All ASO's retained hTLR8 activity (Table 40).

TABLE 40
Assay Results for ASOs based on ASO-182
with different MsPA linker positions

| | hTLR8 | | | | HepG2.2.15 | |
|---|---|---|---|---|---|---|
| | | | | | HBsAg | |
| | | | | Fold Over | EC50 | |
| ASO | 48 hrs (raw signal) | | | ASO-1 | (nM) | CC50 (nM) |
| ASO-1 | 2.23 | ± | 0.24 | 1 | 4.46 | >167 |
| ASO-6 | 2.55 | ± | 0.15 | 1.14 | 7.89 | >167 |
| ASO-182 | 2.50 | ± | 0.13 | 1.12 | 2.41 | >167 |
| ASO-641 | 2.19 | ± | 0.12 | 0.98 | 1.97 | >167 |
| ASO-642 | 2.04 | ± | 0.07 | 0.91 | 1.91 | >167 |
| ASO-643 | 2.44 | ± | 0.10 | 1.09 | 1.85 | >167 |
| ASO-644 | 2.78 | ± | 0.09 | 1.24 | 2.42 | >167 |
| ASO-645 | 2.40 | ± | 0.11 | 1.07 | 2.01 | >167 |
| ASO-646 | 2.58 | ± | 0.10 | 1.16 | 2.45 | >167 |
| ASO-647 | 2.56 | ± | 0.10 | 1.15 | 2.88 | >167 |
| ASO-648 | 2.38 | ± | 0.14 | 1.06 | 2.26 | >167 |
| ASO-649 | 1.97 | ± | 0.03 | 0.88 | 2.51 | >167 |
| ASO-650 | 2.10 | ± | 0.05 | 0.94 | 2.51 | >167 |

### Example 42:21-Mer ASOs with Differing Gap Regions Sizes in ASO-1

Additional 21-mer ASOs were prepared based on ASO-1 hotspots. ASO-629, ASO-630, ASO-631, ASO-632, ASO-633, ASO-634, ASO-635, ASO-636, ASO-637, ASO-638, ASO-639, and ASO-640 were prepared with a gap region as described in Table 41. The ASOs were subjected to the assays described in Example 2 to determine RNAse H-mediated degradation, cytotoxicity, TLR8 pathway activation, and caspase activation (Table 41).

TABLE 41
Assay Results for ASOs based on ASO-1 hotspots with differing gap region sizes

| | | Caspase | Caspase | | | |
|---|---|---|---|---|---|---|
| | | Fold over | Fold over | TLR8 | | |
| | | ASO-1 | ASO-1 | Fold Over | HepG2.2.15 | HepG2.2.15 |
| ASO | Description | 167 nM | 56 nM | ASO-1 | EC50 nM | CC50 nM |
| ASO-1 | | 1 | 1 | | 5.84 | >167 |
| ASO-629 | gapmer_4-10-7 | 0.99 | 1.76 | 0.78 | 10.86 | >167 |
| ASO-630 | gapmer 5-10-6 | 0.83 | 3.65 | 0.84 | 4.688 | >167 |
| ASO-631 | gapmer 6-10-5 | 1.09 | 4.68 | 0.93 | 5.29 | >167 |
| ASO-632 | gapmer 7-10-4 | 0.92 | 4.93 | 0.83 | 3.34 | >167 |
| ASO-633 | gapmer 4-9-8 | 0.39 | 1.14 | 0.84 | 11.47 | >167 |
| ASO-634 | gapmer 5-9-7 | 0.38 | 1.87 | 0.87 | 9.91 | >167 |
| ASO-635 | gapmer 6-9-6 | 0.91 | 4.36 | 0.92 | 8.17 | >167 |
| ASO-636 | gapmer 7-9-5 | 1.16 | 4.44 | 0.99 | 7.93 | >167 |
| ASO-637 | gapmer_8-9-4 | 1.31 | 3.44 | 0.99 | 4.07 | >167 |
| ASO-638 | gapmer 6-8-7 | 0.93 | 0.61 | 0.95 | 20.19 | >167 |
| ASO-639 | gapmer 7-8-6 | 1.01 | 2.19 | 1.01 | 10.50 | >167 |
| ASO-640 | gapmer 5-11-5 | 1.07 | 2.31 | 1.27 | 5.78 | >167 |
| ASO-635 | gapmer 6-9-6 | 0.91 | 4.36 | 0.92 | 8.17 | >167 |

### Example 43:21-Mer ASOs with Modified Wing Lengths

Additional 21-mer ASOs were prepared with modified wing lengths. ASO-776, ASO-777, ASO-778, ASO-779, ASO-780, ASO-781, ASO-782 and ASO-783, were prepared as shown in Table 1 with the wing regions as described in Table 42. The ASOs were subjected to the assays described in Example 2 to determine RNAse H-mediated degradation, cytotoxicity, TLR8 pathway activation and caspase activation. ASO-779 showed improved caspase activity, with all ASOs showing reduced hTLR8 activity as compared to controls ASO-139 and ASO-1 (Table 42)

TABLE 42
Assay Results for 21-mer ASOs with modified wing lengths

| | | Caspase | Caspase | | |
|---|---|---|---|---|---|
| | | Fold | Fold | | |
| | | over | over | | |
| | | ASO-1 | ASO-1 | EC50 | CC50 |
| ASO | Position | 167 nM | 56 nM | nM | nM |
| ASO-776 | gapmer_5-10-6 | 0.99 | 1.05 | 4.92 | >167 |
| ASO-777 | gapmer_6-10-5 | 1.24 | 1.42 | 5.34 | >167 |
| ASO-778 | gapmer_5-10-6 | 1.12 | 1.89 | 9.00 | >167 |
| ASO-779 | gapmer_6-10-5 | 0.57 | 0.32 | 5.80 | >167 |
| ASO-780 | gapmer_5-10-6 | 1.09 | 3.48 | 4.80 | >167 |
| ASO-781 | gapmer_6-10-5 | 1.12 | 3.51 | 5.66 | >167 |
| ASO-782 | gapmer_5-10-6 | 1.15 | 2.42 | 4.93 | >167 |
| ASO-783 | gapmer_6-10-5 | 1.14 | 1.58 | 3.07 | >167 |
| ASO-139 | | 1.40 | 8.78 | 1.11 | >167 |
| ASO-1 | | 1 | 1 | 4.31 | >167 |

### Example 44: Differing LNA Modification Patterns Based on ASO-139

Additional ASOs were prepared based with differing LNA modification patterns based on ASO-139 as shown in Table 43. The ASOs were subjected to the assays described in Example 2 to determine RNAse H-mediated degradation, cytotoxicity, TLR8 pathway activation and caspase activation (Table 43). Removing a LNA from ASO-139 reduced RNase H activity without significant improvements to the caspase profile (Table 43).

TABLE 43
Assay Results for ASOs based on ASO-139
with differing LNA modification patterns

| | | Caspase | Caspase | | |
|---|---|---|---|---|---|
| | | Fold | Fold | | |
| | | over | over | | |
| | | ASO-1 | ASO-1 | EC50 | CC50 |
| ASO | LNA Position | 167 nM | 56 nM | nM | nM |
| ASO-139 | 17, 19, 20 | 1.25 | 3.59 | 1.98 | >167 |
| ASO-696 | 17, 19 | 1.06 | 1.11 | 5.19 | >167 |
| ASO-697 | 17, 20 | 1.05 | 1.45 | 6.988 | >167 |
| ASO-698 | 19, 20 | 0.98 | 1.18 | 5.436 | >167 |
| ASO-699 | 17 | 0.83 | 1.02 | 9.196 | >167 |
| ASO-700 | 19 | 0.87 | 0.85 | 5.514 | >167 |
| ASO-701 | 20 | 0.99 | 1.06 | 8.283 | >167 |
| ASO-1 | | 1 | 1 | 10.3 | >167 |

### Example 45: ASOs with Multiple MsPA Backbone Modifications

ASOs were prepared with one or two MsPA linker backbone modifications as shown in Table 44. The ASOs were subjected to the assays described in Example 2 to determine RNAse H-mediated degradation, cytotoxicity, TLR8 pathway activation and caspase activation (Table 44). Several ASOs in this group such as ASO-579, ASO-581 and ASO-582 significantly improved the caspase profiles without reducing in vitro activities.

TABLE 44
Assay Results for ASOs with multiple MsPA backbone modifications

| | | Caspase | Caspase | | | |
|---|---|---|---|---|---|---|
| | | Fold over | Fold over | TLR8 | | |
| | | ASO-1 | ASO-1 | Fold Over | HepG2.2.15 | HepG2.2.15 |
| | Description | 167 nM | 56 nM | ASO-1 | EC50 nM | CC50 nM |
| ASO-579 | MsPA 6 to 7 | 0.54 | 0.36 | 1.11 | 7.71 | >167 |
| ASO-580 | MsPA 5 to 7 | 0.35 | 0.43 | 0.83 | 2.12 | 63.11 |
| ASO-581 | MsPA 6 to 8 | 0.54 | 0.15 | 0.82 | 7.19 | >167 |
| ASO-582 | MsPA 7 to 9 | 0.36 | 0.18 | 0.87 | 5.21 | >167 |
| ASO-583 | MsPA 8 to 10 | 0.22 | 0.30 | 0.92 | 7.99 | >167 |
| ASO-584 | MsPA 9 to 11 | 0.35 | 0.63 | 1.04 | 15.20 | >167 |
| ASO-585 | MsPA 10 to 12 | 0.28 | 0.26 | 0.97 | 12.78 | >167 |
| ASO-586 | MsPA 11 to 13 | 1.38 | 0.62 | 1.72 | 21.65 | >167 |
| ASO-587 | MsPA 12 to 14 | 1.53 | 0.42 | 1.00 | 15.80 | >167 |
| ASO-588 | MsPA 13 to 15 | 1.12 | 0.50 | 0.54 | 10.96 | >167 |
| ASO-589 | MsPA 14 to 16 | 1.03 | 0.56 | 1.07 | 42.78 | >167 |
| ASO-1 | | 1 | 1 | 1 | 5.84 | >167 |

### Example 46: ASOs with an Abasic Monomer 2′OMeX Modification on the 3′ Wing of ASO-1

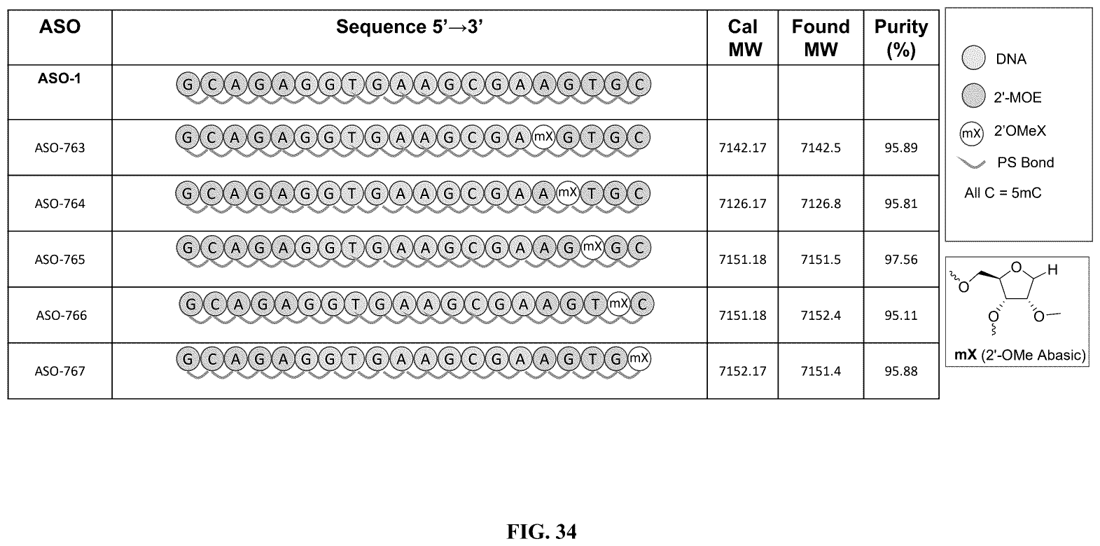

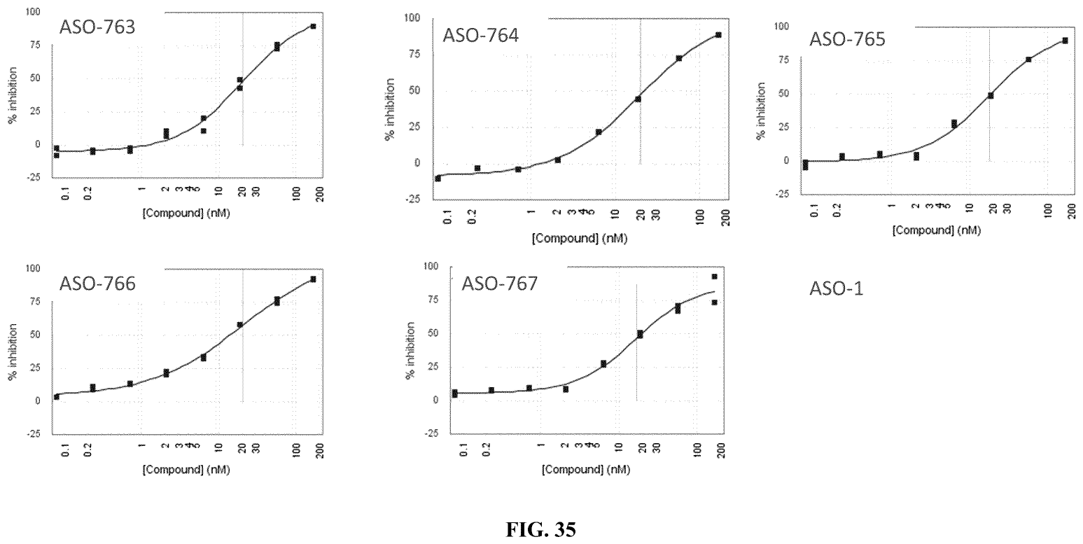

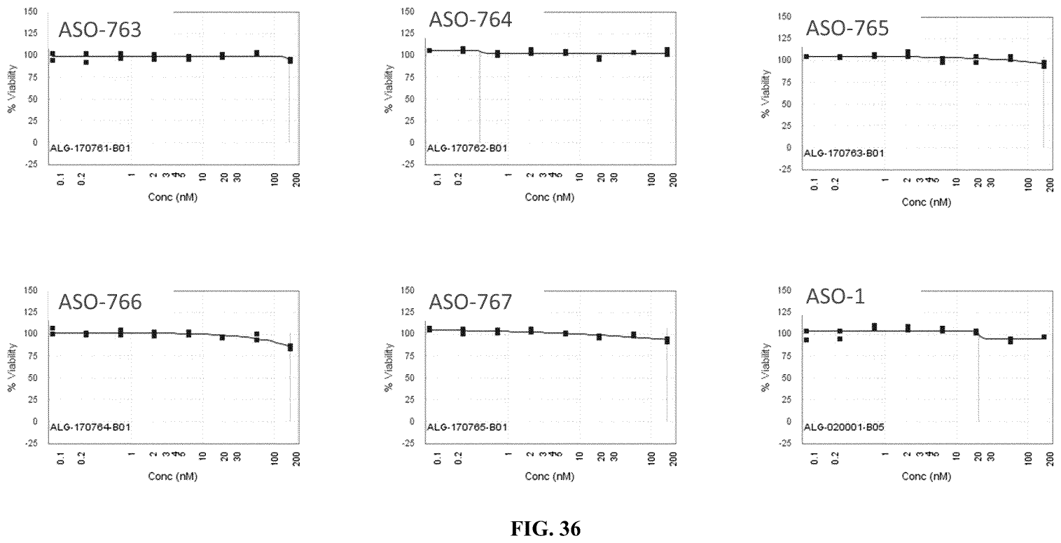

Additional ASOs were prepared as shown in FIG. 34 using a 3′ wing abasic monomer walk based on ASO-1. The ASOs were subjected to the assays described in Example 2 to determine RNAse H-mediated degradation and cytotoxicity (Table 45). The % reduction in RNaseH activity and % viability after treatment of the ASO's at 0.1, 0.2, 1, 2, 3, 4, 5, 10, 20, 30, 100, and 200 nM was assessed (FIG. 35 and FIG. 36).

TABLE 45
Assay Results for ASOs based on ASO-1 with abasic
monomer 2′OMeX modifications at varying positions

| ASO-1 3′ wing | | EC50 | CC50 |
|---|---|---|---|
| abasic monomer walk | Position | nM | nM |
| ASO-763 | 16 | 19.97 | >167 |
| ASO-764 | 17 | 19.66 | >167 |
| ASO-765 | 18 | 17.58 | >167 |
| ASO-766 | 19 | 20.33 | >167 |
| ASO-767 | 20 | 16.529 | >167 |
| ASO-1 | | 10.33 | >167 |

### Example 47: ASOs with an LNA Mismatch Modification Based on ASO-139

ASOs were prepared as shown in Table 46 using a 3′ LNA mismatch approach. The ASOs were subjected to the assays described in Example 2 to determine RNAse H-mediated degradation and cytotoxicity (Table 46).

TABLE 46
Assay Results for ASOs based on ASO-139
with an LNA mismatch at varying positions

| | | Caspase | Caspase | | |
|---|---|---|---|---|---|
| | | Fold over | Fold over | | |
| ASO-139- 3′ | | ASO-1 | ASO-1 | EC50 | CC50 |
| LNA MisMatch | Position | 167 nM | 56 nM | nM | nM |
| ASO-754 | 17-A | 1.84 | 1.14 | 22.63 | >167 |
| ASO-768 | 17-C | 2.50 | 2.27 | 26.76 | >167 |
| ASO-769 | 17-T | 1.17 | 0.80 | 14.53 | >167 |
| ASO-770 | 19-A | 2.35 | 1.31 | 28.08 | >167 |
| ASO-771 | 19-C | 2.24 | 0.31 | 16.42 | >167 |
| ASO-772 | 19-T | 1.86 | 0.41 | 26.65 | >167 |
| ASO-773 | 20-A | 2.10 | 0.41 | 61.31 | >167 |
| ASO-774 | 20-G | 2.78 | 1.53 | 90.09 | >167 |
| ASO-775 | 20-T | 1.73 | 0.31 | 26.13 | >167 |
| ASO-139 | 17, 19, 20 | 1.92 | 1.18 | 1.98 | >167 |
| ASO-1 | | 1 | 1 | 10.3 | >167 |

### Example 48: ASOs Design with a 2′-OMe and LNA Combination Approach

ASOs were prepared using a 2′-OMe and LNA modification combination approach based on parental ASOs ASO-153, ASO-182 and ASO-183. The ASOs were subjected to the assays described in Example 2 to determine RNAse H-mediated degradation, cytotoxicity, TLR8 pathway activation and caspase activation with ASO-272 demonstrating the best in vivo profile (Table 47). Most ASOs in this group showed significant cytotoxicity.

TABLE 47
Assay Results for ASOs based on ASO-153 with a combination
of 2′-OMe and LNA modifications at varying positions

| | | | Caspase | |
|---|---|---|---|---|
| | EC50 | CC50 | Fold/ASO-1 | hTLR8 |
| ASO | nM | nM | at 167 nM | Fold/ASO-1 |
| ASO-269 | 1.707 | 38.66 | 2.2 | 1.17 |
| ASO-270 | 1.78 | 43.2 | 2.3 | 1.08 |
| ASO-271 | 2.1 | 136.99 | | 1.40 |
| ASO-272 | 2.872 | >167 | 0.90 | 1.32 |
| ASO-273 | 1.907 | 33.22 | 3.1 | 1.11 |
| ASO-274 | 1.665 | 40.07 | 1.4 | 1.07 |
| ASO-275 | 2.371 | 62.68 | 4.1 | 1.06 |
| ASO-277 | 2.515 | >167 | 2.1 | 1.13 |
| ASO-278 | 2.635 | 56.31 | 2.8 | 0.79 |
| ASO-279 | 1.467 | 45.51 | 2.9 | 0.85 |
| ASO-280 | 2.203 | >167 | 1.6 | 0.94 |
| ASO-281 | 1.531 | 66.88 | 2.3 | 1.36 |
| ASO-282 | 1.11 | 53.19 | 3.1 | 0.89 |
| ASO-283 | 0.86 | 33.51 | 3.3 | 1.00 |
| ASO-1 | 5.2 | >167 | 1.0 | 1.0 |

### Example 49: ASOs Designed with Stereodefined Dimers in the 5′ Wings

ASOs were designed with stereodefined dimers in the 5′ wings based on ASO-6 to prepare ASO-426, ASO-427, ASO-428, ASO-429, ASO-430, ASO-431, and ASO-432 (Table 1.). The ASOs were subjected to the assays described in Example 2 to determine RNAse H-mediated degradation, cytotoxicity, and TLR8 pathway activation. None of the ASOs showed improved RNase H activity while most showed significant cytotoxicity (Table 48).

TABLE 48
Assay Results for ASO's based on ASO-6 with
stereodefined dimers in the 5′ wings

| | EC50 | CC50 | hTR8 Fold |
|---|---|---|---|
| ASO | nM | nM | Over ASO-1 |
| ASO-426 | 7.16 | 38.66 | 1.16 |
| ASO-427 | 6.92 | 43.22 | 0.96 |
| ASO-428 | 6.30 | 136.99 | 1.03 |
| ASO-429 | 6.30 | >167 | 0.97 |
| ASO-430 | 5.58 | 33.21 | 0.92 |
| ASO-431 | 7.94 | 40.07 | 1.06 |
| ASO-432 | 5.95 | 62.68 | 0.89 |
| ASO-1 | 5.2 | >167 | |

### Example 50: ASOs Designed with scpBNA Modifications Based on ASO-6

ASOs were designed with scpBNA modifications based on the parental ASO, ASO-6. The ASOs were subjected to the assays described in Example 2 to determine RNAse H-mediated degradation, cytotoxicity, TLR8 pathway activation, and caspase activation, with scpBNA modifications not improving the toxicity profile (Table 49) and (Table 50). Most ASOs showed worsened cytotoxicity.

TABLE 49
Assay Results for ASOs based on ASO-6 with
scpBNA modifications at varying positions

| | | | Caspase Fold | Caspase Fold | |
|---|---|---|---|---|---|
| | EC50 | CC50 | over ASO-1 | over ASO-1 | hTLR8 Fold |
| ASO | nM | nM | 167 nM | 56 nM | Over ASO-1 |
| ASO-479 | 2.24 | >167 | 1.05 | 1.33 | 0.96 |
| ASO-480 | 2.77 | 20.26 | 1.16 | 1.51 | 1.23 |
| ASO-481 | 1.39 | 43.33 | 1.14 | 1.45 | 1.32 |
| ASO-482 | 1.39 | 58.86 | 1.10 | 1.40 | 1.06 |
| ASO-483 | 2.29 | 85.47 | 1.08 | 1.77 | 1.19 |
| ASO-484 | 2.17 | 62.26 | 1.07 | 1.75 | 1.34 |
| ASO-485 | 2.61 | >167 | 1.09 | 1.78 | 1.13 |
| ASO-486 | 3.39 | >167 | 1.16 | 1.90 | 0.67 |
| ASO-487 | 3.06 | 57.59 | 1.03 | 1.37 | 0.87 |
| ASO-488 | 10.33 | 65.16 | 1.02 | 1.29 | 0.76 |
| ASO-489 | 1.98 | >167 | 0.98 | 1.27 | 0.95 |
| ASO-490 | 2.18 | 66.73 | 1.10 | 1.54 | 0.77 |

TABLE 50
Assay Results for ASOs based on ASO-6 with
scpBNA modifications at varying positions

| | | | Caspase Fold | Caspase Fold | |
|---|---|---|---|---|---|
| | EC50 | CC50 | over ASO-1 | over ASO-1 | hTLR8 Fold |
| ASO | nM | nM | 167 nM | 56 nM | Over ASO-1 |
| ASO-492 | 1.49 | 40.34 | 1.17 | 1.63 | 1.04 |
| ASO-493 | 1.46 | 35.85 | 1.19 | 1.68 | 1.03 |
| ASO-494 | 2.33 | 83.71 | 1.08 | 1.46 | 0.94 |
| ASO-495 | 1.41 | >167 | 1.12 | 1.62 | 1.01 |
| ASO-496 | 2.43 | 68.12 | 1.01 | 1.29 | 0.82 |
| ASO-497 | 0.86 | 55.31 | 1.07 | 1.35 | 0.96 |
| ASO-498 | 2.27 | 62.37 | 1.10 | 1.38 | 1.14 |
| ASO-499 | 1.50 | 22.79 | 1.15 | 1.64 | 0.86 |
| ASO-500 | 1.49 | 52.26 | 1.14 | 1.56 | 0.63 |
| ASO-501 | 1.923 | 102.39 | 1.01 | 1.27 | 1.04 |
| ASO-502 | 1.55 | 55.12 | 1.09 | 1.51 | 1.04 |
| ASO-1 | 5.427 | >167 | 1 | 1 | 1 |

### Example 51: ASOs Designed with scpBNA Modifications and LNA Modifications

ASOs were designed with scpBNA and LNA modifications. The ASOs were subjected to the assays described in Example 2 to determine RNAse H-mediated degradation, cytotoxicity, TLR8 pathway activation, and caspase activation, with scpBNA and LNA modifications not improving the toxicity profile (Table 51).

TABLE 51
Assay Results for ASOs with scpBNA and
LNA modifications at varying positions

| | | | Caspase Fold | Caspase Fold | |
|---|---|---|---|---|---|
| | EC50 | CC50 | over ASO-1 | over ASO-1 | hTR8 Fold |
| ASO | nM | nM | 167 nM | 56 nM | Over ASO-1 |
| ASO-503 | 7.16 | >167 | 1.15 | 1.53 | 1.13 |
| ASO-504 | 6.92 | >167 | 1.09 | 1.41 | 1.23 |
| ASO-505 | 6.30 | >167 | 1.12 | 1.44 | 1.21 |
| ASO-506 | 6.30 | >167 | 1.07 | 1.35 | 1.13 |
| ASO-507 | 5.58 | >167 | 1.17 | 1.07 | 1.23 |
| ASO-508 | 7.94 | >167 | 1.13 | 1.39 | 1.08 |
| ASO-1 | 5.427 | >167 | 1 | 1 | 1 |

### Example 52: ASOs Designed with ocp/omcp/xylo Modification in the Gap Region

ASOs were designed with ocp, omcp, or xylo modification in the gap region as shown in Table 1. The ASO's were subjected to the assays described in Example 2 to determine RNAse H-mediated degradation, cytotoxicity, TLR8 pathway activation, and caspase activation, with ocp, omcp, and xylo modifications showing improved safety profile but not improving RNase H activity (modifications not improving the toxicity profile (Table 52).

TABLE 52
Assay Results for ASOs with ocp/omcp/xylo
modification in the gap region

| | | | Caspase Fold | Caspase Fold | |
|---|---|---|---|---|---|
| | EC50 | CC50 | over ASO-1 | over ASO-1 | hTR8 Fold |
| ASO | nM | nM | 167 nM | 56 nM | Over ASO-1 |
| ASO-300 | 160.31 | >167 | 0.56 | 0.88 | 0.55 |
| ASO-301 | 166.4 | >167 | 0.50 | 0.74 | 0.97 |
| ASO-302 | 76.04 | >167 | 0.53 | 0.82 | 1.09 |
| ASO-303 | 164.94 | >167 | 0.48 | 0.68 | 0.95 |
| ASO-304 | 164.06 | >167 | 0.51 | 0.77 | 0.82 |
| ASO-305 | 164.58 | >167 | 0.56 | 0.84 | 0.92 |
| ASO-306 | 162.87 | >167 | 0.52 | 0.81 | 0.75 |
| ASO-307 | 166.33 | >167 | 0.56 | 0.88 | 1.43 |
| ASO-308 | 14.95 | >167 | 0.83 | 1.05 | 1.23 |
| ASO-1 | 7.29 | >167 | 1 | 1 | 1 |

### Example 53: ASOs Designed with Stereo-Defined R—PS Backbone Based on ASO-6

ASOs were designed with a stereo-defined R—PS modification based on ASO-6. The ASOs were subjected to the assays described in Example 2 to determine RNAse H-mediated degradation, cytotoxicity, TLR8 pathway activation, and caspase activation, with stereo-defined R—PS backbone modification showing no significant change (Table 53).

TABLE 53
Assay Results for ASOs based on ASO-6 with stereo-defined
R-PS backbone modifications at varying positions

| | | | Caspase Fold | Caspase Fold | |
|---|---|---|---|---|---|
| | EC50 | CC50 | over ASO-1 | over ASO-1 | hTR8 Fold |
| ASO | nM | nM | 167 nM | 56 nM | Over ASO-1 |
| ASO-6 | 5.49 | >167 | 0.99 | 1.03 | 1.17 |
| ASO-227 | 6.47 | >167 | 0.99 | 0.99 | 1.23 |
| ASO-228 | 8.42 | >167 | 0.98 | 1.11 | 1.18 |
| ASO-229 | 9.54 | >167 | 0.92 | 1.02 | 1.23 |
| ASO-230 | 6.7 | >167 | 0.95 | 1.18 | 1.19 |
| ASO-231 | 9.44 | >167 | 0.96 | 1.11 | 1.26 |
| ASO-232 | 9.52 | >167 | 1.07 | 1.13 | 1.21 |
| ASO-233 | 9.09 | >167 | 1.11 | 1.17 | 1.20 |
| ASO-234 | 6.31 | >167 | 1.14 | 1.24 | 1.10 |
| ASO-235 | 7.35 | >167 | 1 | 1.15 | 1.42 |
| ASO-236 | 11.26 | >167 | 0.97 | 1.15 | 1.28 |
| ASO-247 | 11.84 | >167 | 1.12 | 1.19 | 1.25 |
| ASO-248 | 9.94 | >167 | 1.18 | 1.26 | 1.22 |
| ASO-1 | 7.29 | >167 | 1 | 1 | 1 |

### Example 54: ASOs Designed with scpBNA Modifications Based on ASO-144 or ASO-222

ASOs were designed with a scpBNA modification based on ASO-144 or ASO-222 as shown in Table 1 for ASO-144 and Table 54 and for ASO-222. The ASOs were subjected to the assays described in Example 2 to determine caspase activation. ASOs based on ASO-144 did not show an improved profile (Table 54). When scpBNA replaces LNA modifications, there is no improvement in the toxicity profile (Table 55).

TABLE 54
Assay Results for ASOs based on ASO-144 with
scpBNA modifications at varying positions

| | Caspase Fold | Caspase Fold |
|---|---|---|
| | over ASO-1 | over ASO-1 |
| ASO | 167 nM | 56 nM |
| ASO-433 | 1.48 | 2.55 |
| ASO-434 | 1.85 | 5.06 |
| ASO-435 | 1.52 | 4.65 |
| ASO-436 | 1.56 | 3.59 |
| ASO-437 | 1.49 | 3.59 |
| ASO-438 | 1.75 | 4.05 |
| ASO-439 | 1.68 | 4.15 |
| ASO-440 | 1.59 | 5.10 |
| ASO-441 | 2.87 | 6.39 |
| ASO-442 | 1.81 | 3.62 |
| ASO-443 | 1.91 | 3.13 |
| ASO-444 | 1.85 | 3.68 |
| ASO-445 | 1.59 | 3.85 |
| ASO-446 | 2.24 | 2.93 |
| ASO-447 | 2.57 | 2.44 |
| ASO-448 | 2.27 | 4.90 |
| ASO-449 | 1.59 | 3.69 |
| ASO-450 | 2.15 | 4.81 |
| ASO-451 | 2.10 | 5.06 |
| ASO-452 | 1.77 | 5.08 |
| ASO-453 | 1.55 | 3.50 |
| ASO-454 | 1.81 | 4.28 |
| ASO-455 | 1.84 | 3.11 |
| ASO-456 | 1.64 | 3.68 |
| ASO-1 | 1 | 1 |

TABLE 55
Assay Results for ASOs based on ASO-222 with
scpBNA modifications at varying positions

| | Caspase Fold | Caspase Fold |
|---|---|---|
| | over ASO-1 | over ASO-1 |
| ASO | 167 nM | 56 nM |
| ASO-457 | 1.36 | 2.64 |
| ASO-458 | 1.31 | 3.28 |
| ASO-459 | 1.45 | 3.27 |
| ASO-460 | 1.33 | 2.29 |
| ASO-461 | 1.31 | 2.89 |
| ASO-462 | 1.99 | 4.34 |
| ASO-1 | 1 | 1 |

### Example 55: ASOs Designed with LNA Walk Based on ASO-1

ASOs were designed using an LNA walk based on the parental strain ASO-1. The ASOs were subjected to the assays described in Example 2 to determine RNAse H-mediated degradation, cytotoxicity, TLR8 pathway activation, and caspase activation. ASO-510, ASO-514, ASO-528, and ASO-532, improved efficacy and safety as compared to ASO-1, however most ASOs showed significant reduction of hTLR8 agonist activity (Table 56).

TABLE 56
Assay Results for ASOs based on ASO-1 with
LNA modifications at varying positions

| | | | Caspase Fold | Caspase Fold | |
|---|---|---|---|---|---|
| | EC50 | CC50 | over ASO-1 | over ASO-1 | hTLR8 Fold |
| ASO | nM | nM | 167 nM | 56 nM | Over ASO-1 |
| ASO-4 | 14.06 | >167 | 0.60 | 1.10 | 0.69 |
| ASO-509 | 12.04 | >167 | 0.65 | 0.59 | 0.51 |
| ASO-510 | 7.18 | >167 | 0.16 | 0.50 | 0.43 |
| ASO-511 | 10.77 | >167 | 0.42 | 0.97 | 0.45 |
| ASO-512 | 5.38 | >167 | 0.77 | −0.22 | 0.44 |
| ASO-513 | 11.80 | >167 | 0.82 | 0.58 | 0.45 |
| ASO-514 | 4.32 | >167 | 0.30 | 0.37 | 0.47 |
| ASO-515 | 5.20 | >167 | 1.02 | 1.41 | 0.51 |
| ASO-516 | 6.30 | >167 | 0.71 | 0.34 | 0.49 |
| ASO-517 | 3.49 | >167 | 1.64 | 1.73 | 0.54 |
| ASO-518 | 13.21 | >167 | 2.66 | 5.00 | 0.45 |
| ASO-519 | 2.71 | >167 | 1.42 | 1.77 | 0.42 |
| ASO-520 | 2.00 | >167 | 1.70 | 2.36 | 0.55 |
| ASO-521 | 2.08 | >167 | 2.5 | 2.9 | 0.50 |
| ASO-522 | 1.51 | >′167 | 2.2 | 2.5 | 0.46 |
| ASO-523 | 4.78 | >′167 | 1.9 | 1.4 | 0.46 |
| ASO-524 | 14.64 | >′167 | 0.8 | 0.6 | 0.47 |
| ASO-525 | 18.70 | >′167 | 1.21 | 0.58 | 0.53 |
| ASO-526 | 8.30 | >′167 | 1.26 | 0.83 | 0.46 |
| ASO-527 | 5.06 | >′167 | 1.34 | 0.50 | 0.43 |
| ASO-528 | 5.41 | >′167 | 0.57 | 0.69 | 0.45 |
| ASO-529 | 6.00 | >′167 | 0.75 | 0.57 | 0.45 |
| ASO-530 | 9.22 | >′167 | 1.08 | 0.16 | 0.41 |
| ASO-531 | 5.63 | >′167 | 0.88 | 0.63 | 0.44 |
| ASO-532 | 5.45 | >′167 | 0.47 | 0 | 0.39 |
| ASO-533 | 7.13 | >′167 | 2.08 | 0.51 | 0.45 |
| ASO-534 | 3.92 | >′167 | 2.06 | 1.12 | 0.44 |
| ASO-535 | 4.85 | >′167 | 1.65 | 0.63 | 0.43 |
| ASO-1 | 4.94 | >′167 | 1 | 1 | 1 |

### Example 56: ASOs Designed with Luxna Modifications in the Gap Region Based on ASO-174

ASO's were designed with a Luxna modification in the gap region based on ASO-174. The ASO's were subjected to the assays described in Example 2 to determine RNAse H-mediated degradation, cytotoxicity, and caspase activation. Most ASOs in this group showed worse in vitro toxicity. ASO-540 reduced toxicity but became inactive (Table 57).

TABLE 57
Assay Results for ASOs based on ASO-174 with
Luxna modifications in the gap region

| | | | Caspase Fold | Caspase Fold |
|---|---|---|---|---|
| | EC50 | CC50 | over ASO-1 | over ASO-1 |
| ASO | nM | nM | 167 nM | 56 nM |
| ASO-174 | 2.39 | >167 | 2.14 | 3.61 |
| ASO-536 | 1.95 | 80.46 | 1.53 | 4.09 |
| ASO-537 | 2.13 | 165.93 | 1.58 | 3.95 |
| ASO-538 | 2.67 | >167 | 1.53 | 3.61 |
| ASO-539 | 2.55 | >167 | 1.81 | 2.41 |
| ASO-540 | >167 | >167 | 0 | 0 |
| ASO-541 | 5.89 | >167 | 1.64 | 2.84 |
| ASO-542 | 3.77 | >167 | 2.20 | 3.47 |
| ASO-543 | 2.05 | >167 | 1.77 | 3.01 |
| ASO-544 | 3.95 | >167 | 1.53 | 2.75 |
| ASO-1 | 4.94 | >167 | 1 | 1 |

### Example 57: ASOs Designed with Stereo-Defined S and R—PS Backbone at 3′ End Flanks Based on ASO-6

ASOs were designed with a stereo-defined R—PS modification or S—PS modification at the 3′ end flanks based on ASO-6. The ASOs were subjected to the assays described in Example 2 to determine TLR8 pathway activation and caspase activation, with ASO-463, ASO-464, ASO-466, and ASO-469, showing significantly improved in vitro safety profiles (Table 58).

TABLE 58
Assay Results for ASOs based on ASO-6 with stereo-
defined S and R-PS backbone at 3′ end flanks

| | Caspase Fold | Caspase Fold | |
|---|---|---|---|
| | over ASO-1 | over ASO-1 | hTLR8 Fold |
| ASO | 167 nM | 56 nM | Over ASO-1 |
| ASO-463 | 0.43 | 0.04 | 1.04 |
| ASO-464 | 0.63 | 0.82 | 1.13 |
| ASO-465 | 0.70 | 2.34 | 0.94 |
| ASO-466 | 0.64 | 0.86 | 0.99 |
| ASO-467 | 0.67 | 1.78 | 0.96 |
| ASO-468 | 0.77 | 2.35 | 0.90 |
| ASO-469 | 0.64 | 0.64 | 0.85 |
| ASO-1 | 1 | 1 | 1 |

### Example 58: ASOs Designed with 2′-omcp Modification in Position 6 and/or 8

ASOs were designed with a 2omcp modification in position 6, position 8, or position 6 and 8 in the parental strains ASO-174, ASO-176, and ASO-182. The ASOs were subjected to the assays described in Example 2 to determine TLR8 pathway activation and caspase activation, with ASO-475 showing an improved profile (Table 59).

TABLE 59
Assay Results for ASOs based on ASO-174, ASO-176, and ASO-
182 with 2′-omcp modification in position 6 and/or 8

| | Caspase Fold | Caspase Fold | |
|---|---|---|---|
| | over ASO-1 | over ASO-1 | hTLR8 Fold |
| Description | 167 nM | 56 nM | Over ASO-1 |
| ASO-470 | 1.28 | 6.20 | 1.18 |
| ASO-471 | 0.78 | 4.43 | 0.95 |
| ASO-472 | 0.59 | 1.69 | 0.80 |
| ASO-473 | 0.99 | 4.89 | 0.86 |
| ASO-474 | 0.85 | 2.63 | 0.78 |
| ASO-475 | 0.54 | 0.81 | 0.72 |
| ASO-476 | 1.25 | 4.92 | 0.90 |
| ASO-477 | 0.64 | 1.51 | 1.24 |
| ASO-478 | 1.08 | 3.06 | 1.13 |
| ASO-1 | 1 | 1 | |

### Example 59: ASOs 2′-OMe Abasic Monomer in the Gap and LNA Modifications in Varying Positions

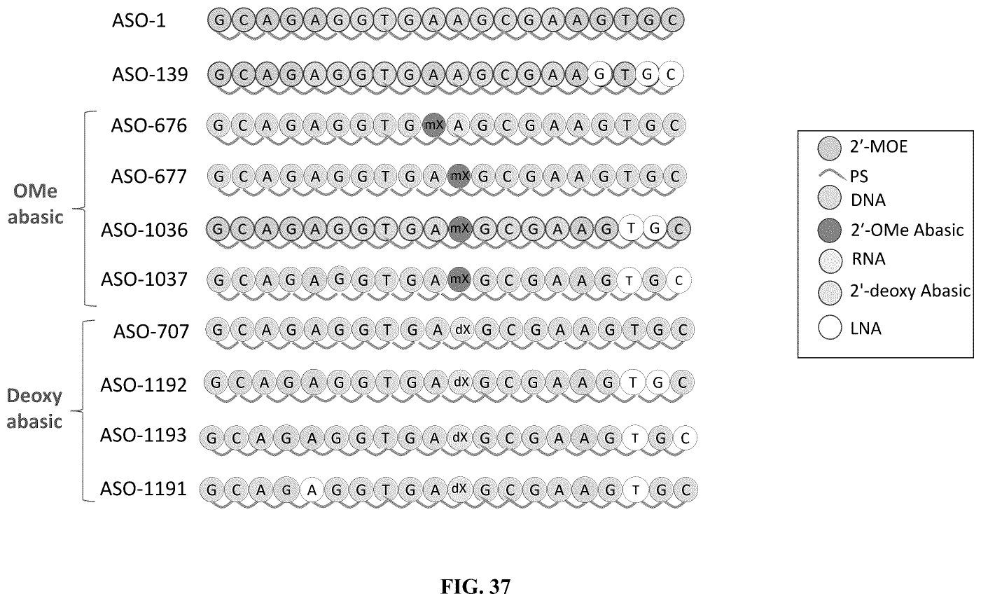

To assess the effect of 2′-OMe abasic monomer and LNA modifications, ASO-1 was used as the parental ASO and ASO-676, ASO-677, ASO-1036, and ASO-1037 were prepared (FIG. 37).

The ASOs were subjected to the assays described in Example 2 to determine RNAse H-mediated degradation, cytotoxicity, TLR8 pathway activation, and caspase activation as compared to controls ASO-1 and ASO-139 (Table 60 and Table 61).

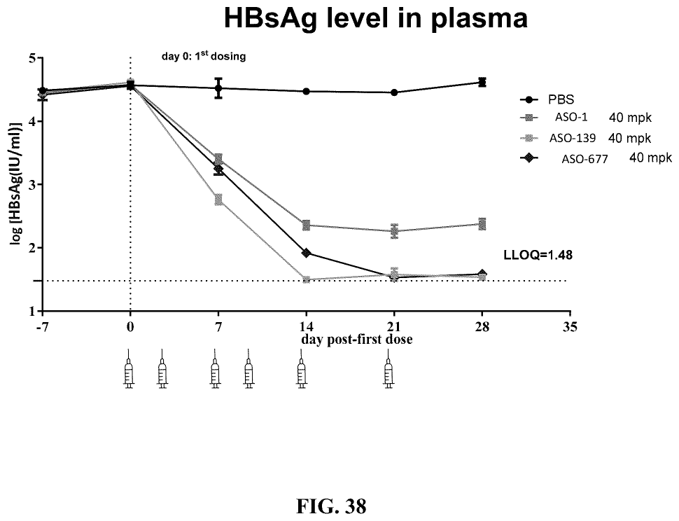

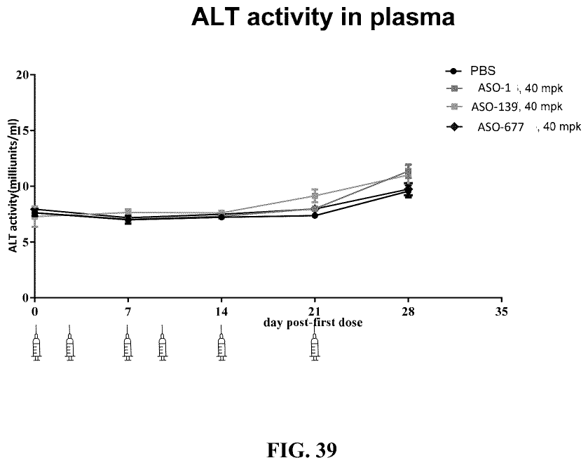

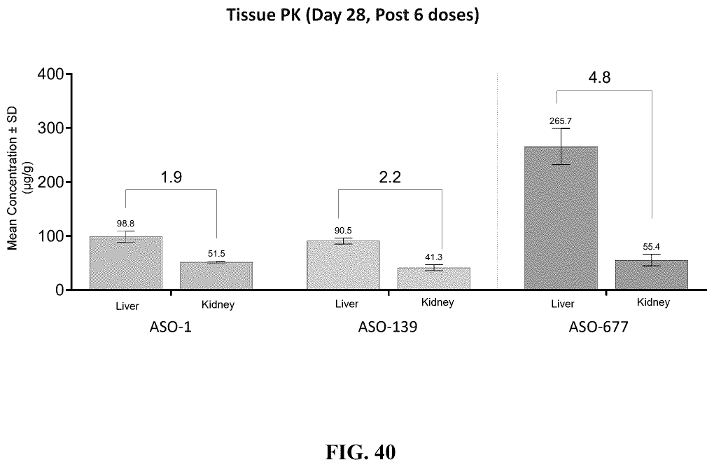

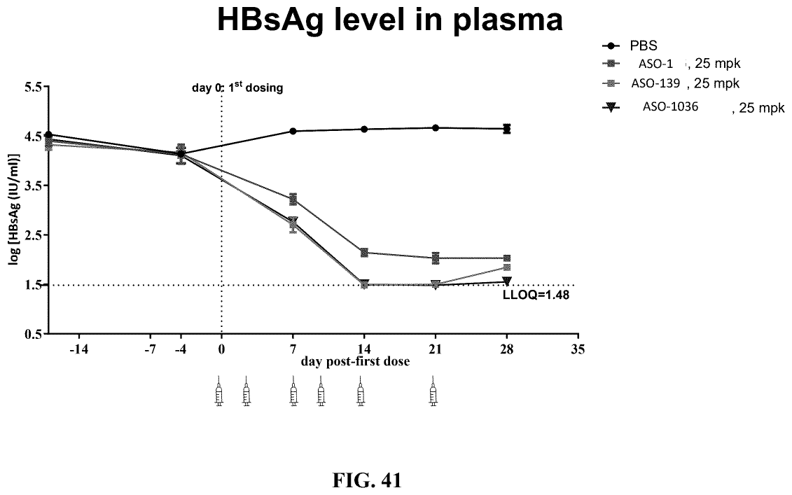

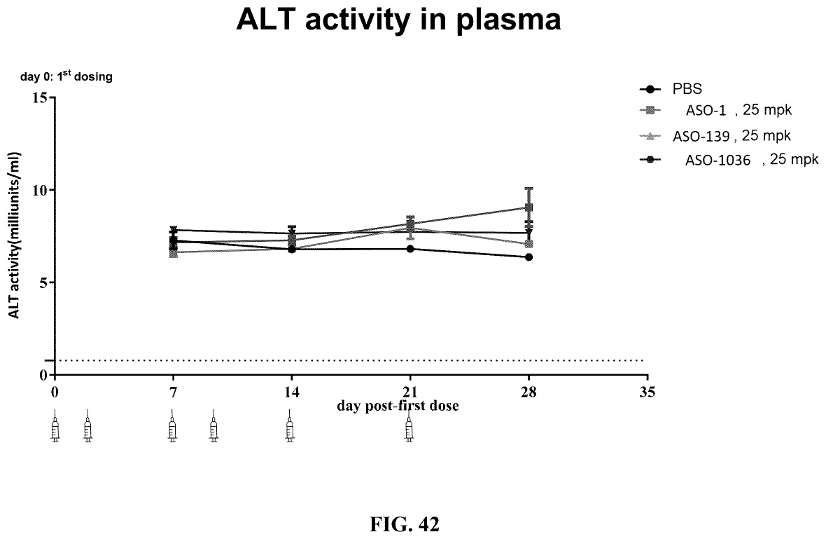

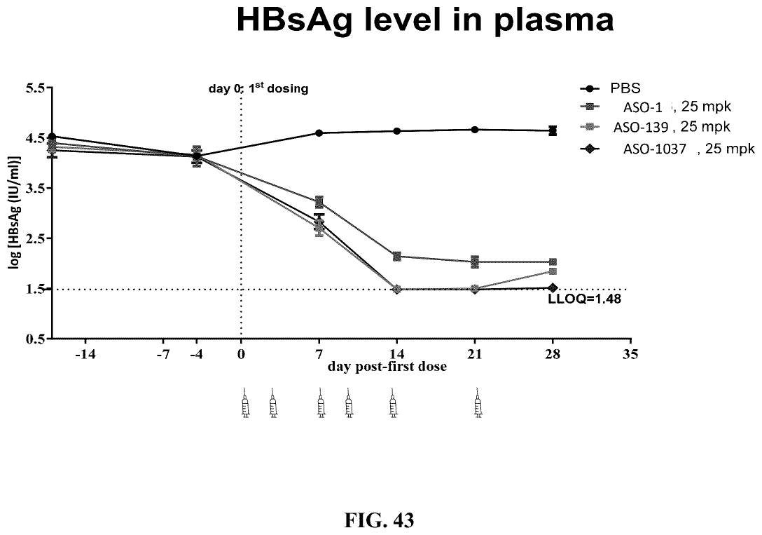

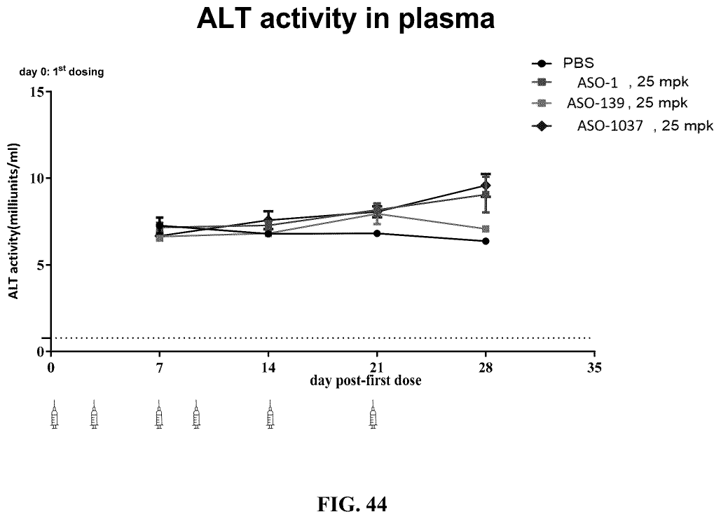

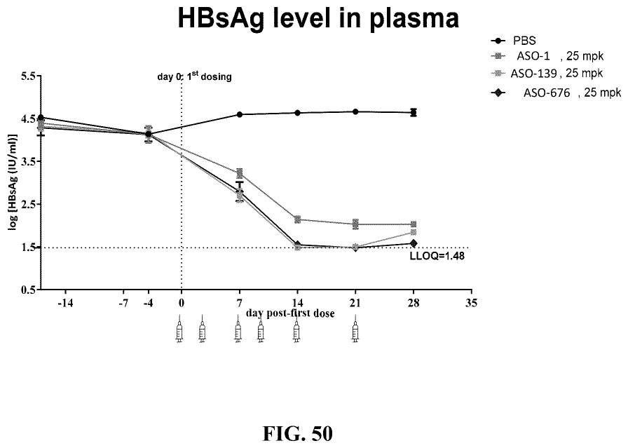

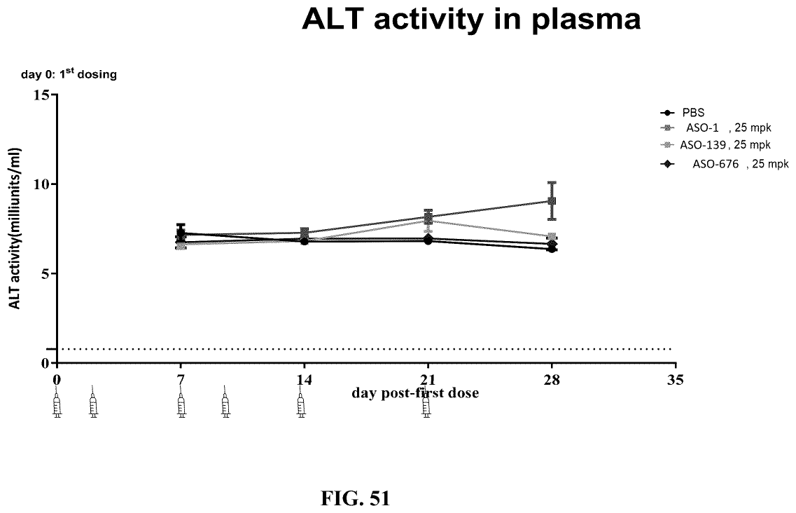

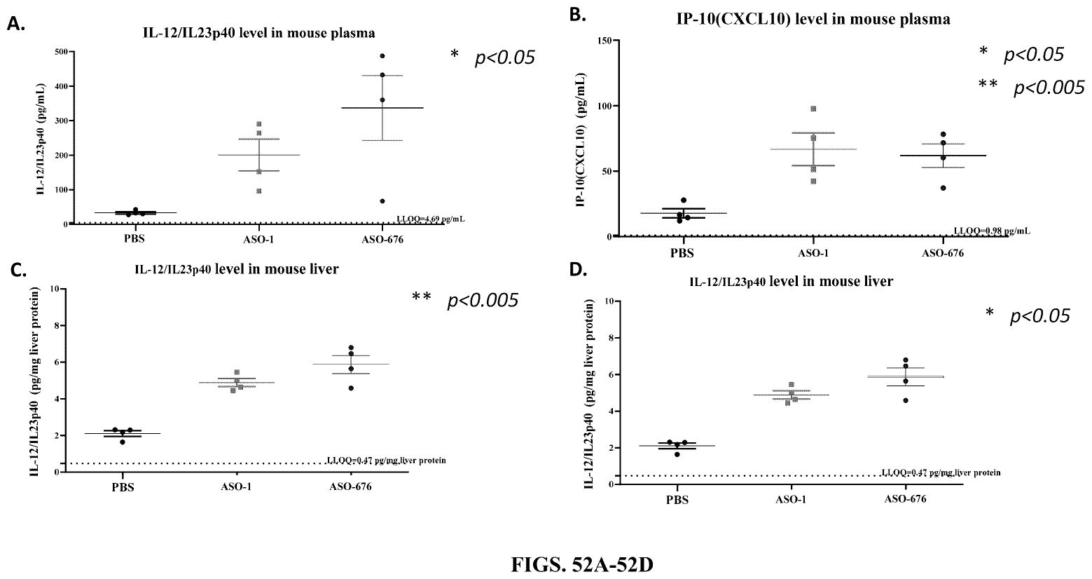

All ASOs show improved caspase profiles and hTLR8 binding activity compared to ASO-1 and ASO-139 (Table 60 and Table 61). ASO-1036 and ASO-1037 showed improved RNase H activity as compared to ASO-1 (Table 61). All ASOs showed better in vivo potency than ASO-1 in reducing HBsAg levels in plasma and there was no ALT elevation for any ASO (FIG. 38, FIG. 39, FIG. 41, FIG. 42, FIG. 43, FIG. 44, FIG. 50, and FIG. 51). ASO-676 showed increased tissue concentration in the liver and improved liver-to-kidney ratio relative to ASO-1 and ASO-139 (FIG. 40). ASO-676 showed similar cytokine profile in hTLR8 knock in mice plasma and liver relative to ASO-1 (FIG. 52). ASO-677 did not result in significantly different cytokine profile relative to ASO-1 (FIG. 63).

TABLE 60
Assay Results for ASOs based on ASO-1 with 2′-
OMe abasic monomer modification at position 10 or 11

| | | | | | | Caspase 3/7 | |
|---|---|---|---|---|---|---|---|
| | | hTLR8 | hTLR8 | | | Fold | Fold |
| | | Fold | Binding | HepG2.2.15 | | over | over |
| | Modifi- | Over | Kd | EC50 | CC50 | ASO-1 | ASO-1 |
| | cation | ASO-1 | (nM) | (nM) | (nM) | 167 nM | 56 nM |
| ASO-1 | | 1 | 1.07 | 8.6 | >167 | 1 | 1 |
| ASO-676 | mX 10 | 0.88 | — | 15.08 | >167 | 0.68 | 0.34 |
| ASO-677 | mX 11 | 0.95 | 1.58 | 9.2 | >167 | 0.58 | 0.34 |

TABLE 61
Assay Results for ASOs based on ASO-1 with 2′-OMe
abasic monomer modification at position 11 and
LNA modifications at positions 18, 19, or 20

| | | Caspase | Caspase | | | TLR8 |
|---|---|---|---|---|---|---|
| | | Fold over | Fold over | | | Fold |
| | | ASO-1, | ASO-1, | EC50 | CC50 | over |
| ASO | Position | 167 nM | 56 nM | nM | nM | ASO-1 |
| ASO-1 | | 1 | 1 | 8.6 | >167 | 1 |
| ASO-139 | LNA 17, | 1.9 | 1.36 | 1.8 | >167 | 0.8 |
| | 19, 20 | | | | | |
| ASO-1036 | mX 11 LNA | 0.65 | 0.40 | 1.08 | >167 | 1.00 |
| | 18, 19 | | | | | |
| ASO-1037 | mX 11 LNA | 0.76 | 0.58 | 2.39 | >167 | 1.07 |
| | 18, 20 | | | | | |

### Example 60: ASOs 2′-Deoxy Abasic Monomer in the Gap and LNA Modifications in Varying Positions

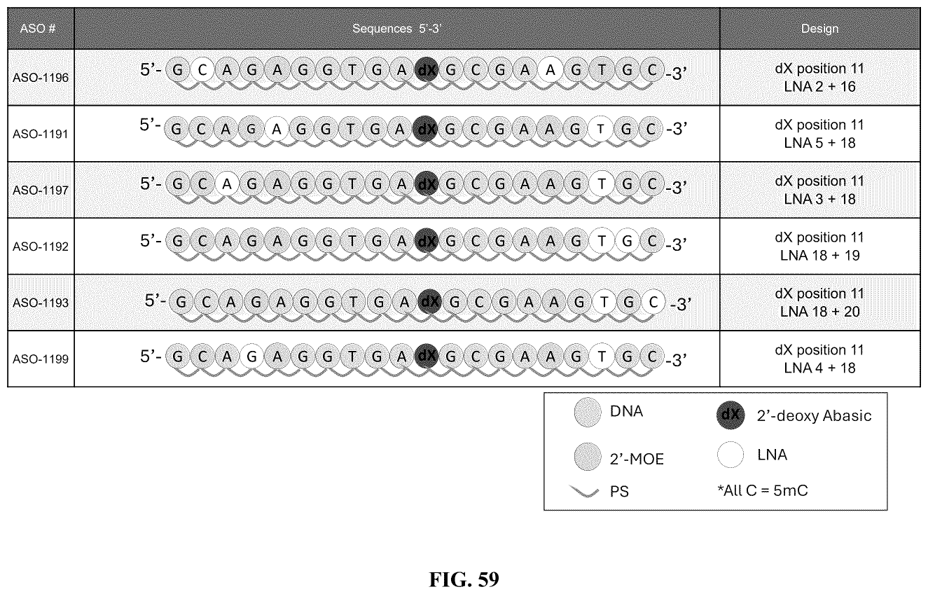

To assess the effect of 2′-deoxy abasic monomers and LNA modifications, ASO-1 was used as the parental ASO and ASO-707, ASO-1191, ASO-1192, and ASO-1193 were prepared (FIG. 37). Additional ASOs based on the parental construct ASO-707, including those shown in FIGS. 27 and 59, were tested as well. The ASOs were subjected to the assays described in Example 2 to determine RNAse H-mediated degradation, cytotoxicity, TLR8 pathway activation, and caspase activation as compared to controls ASO-1 and ASO-139 (see Tables 62-65).

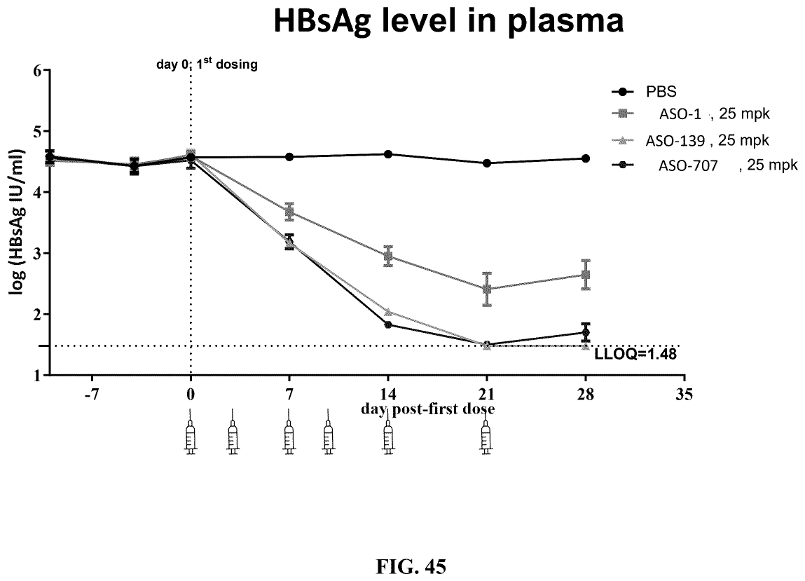

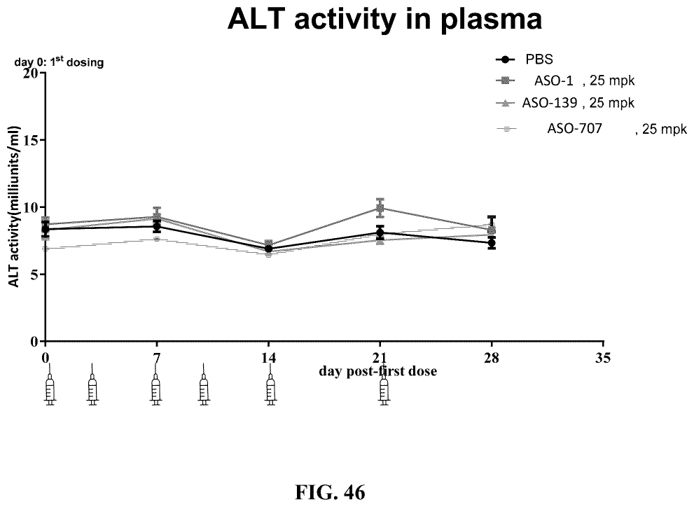

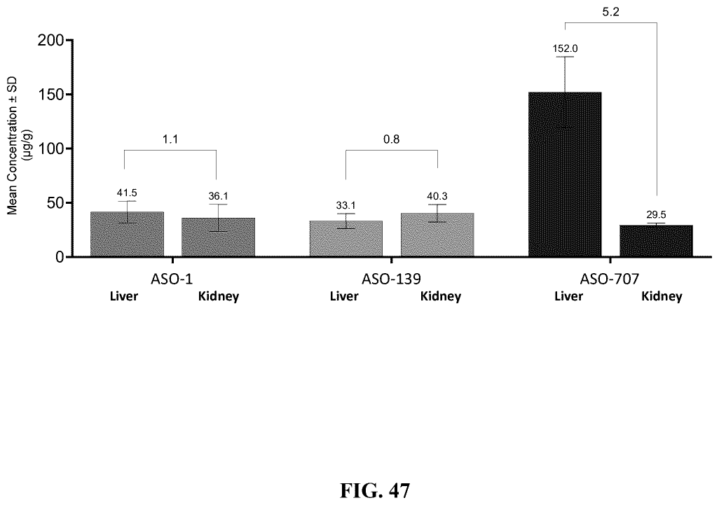

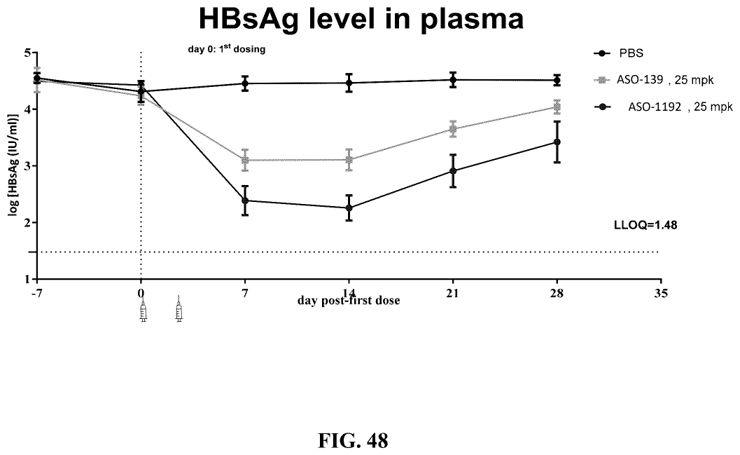

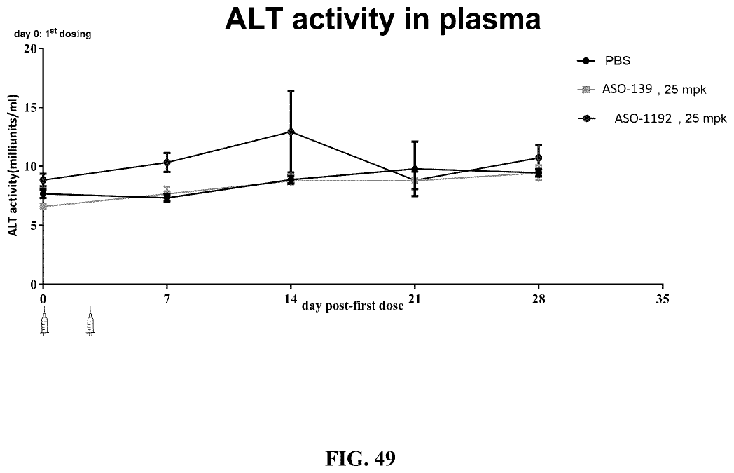

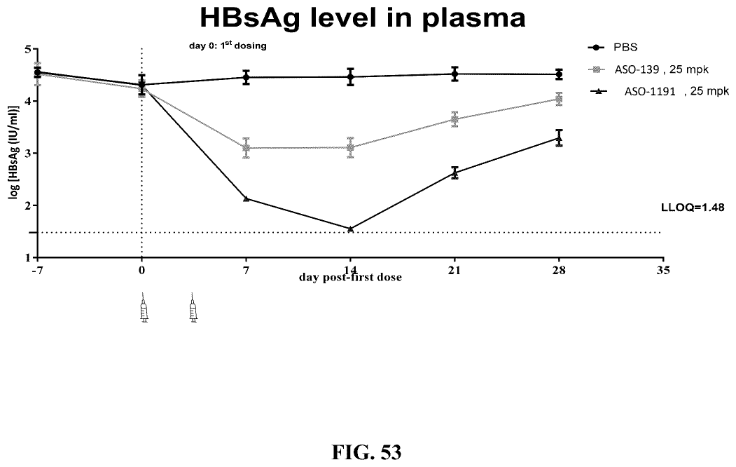

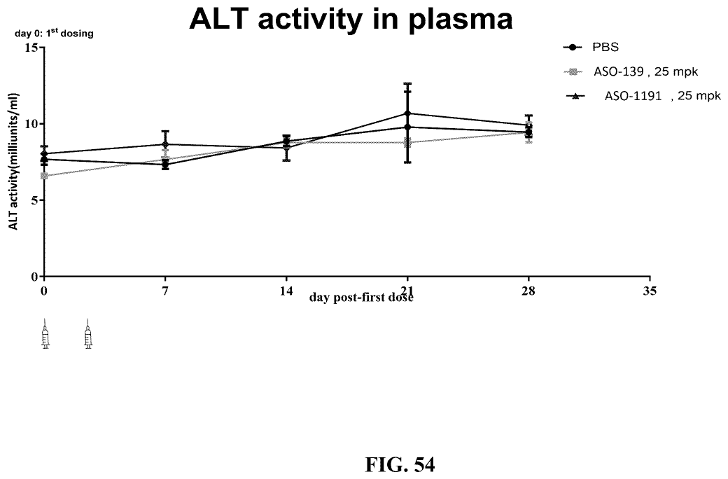

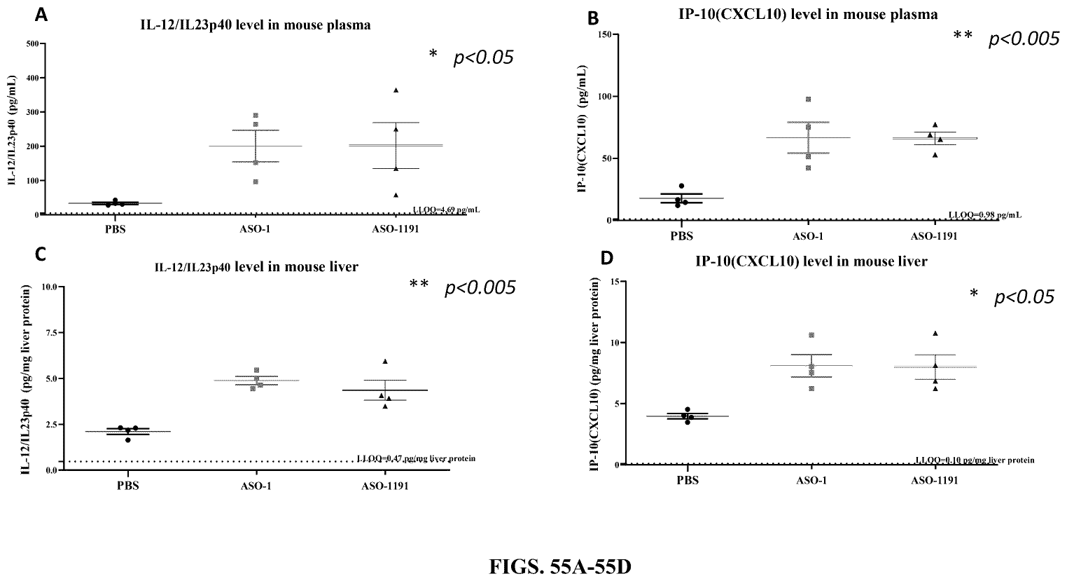

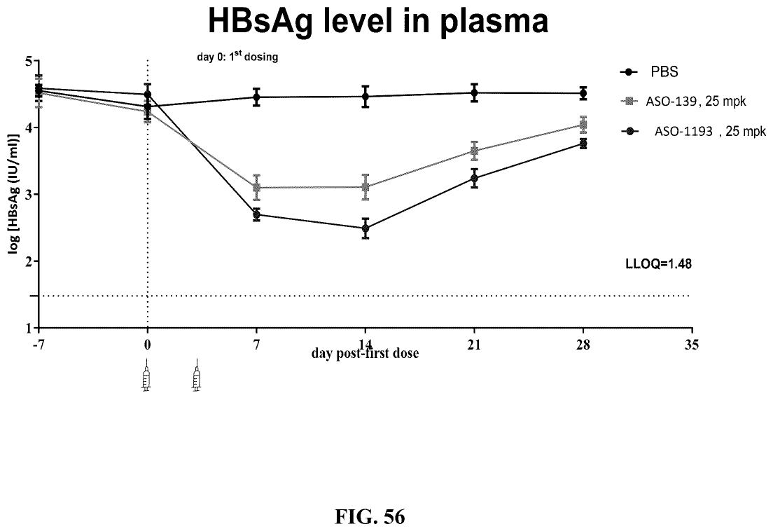

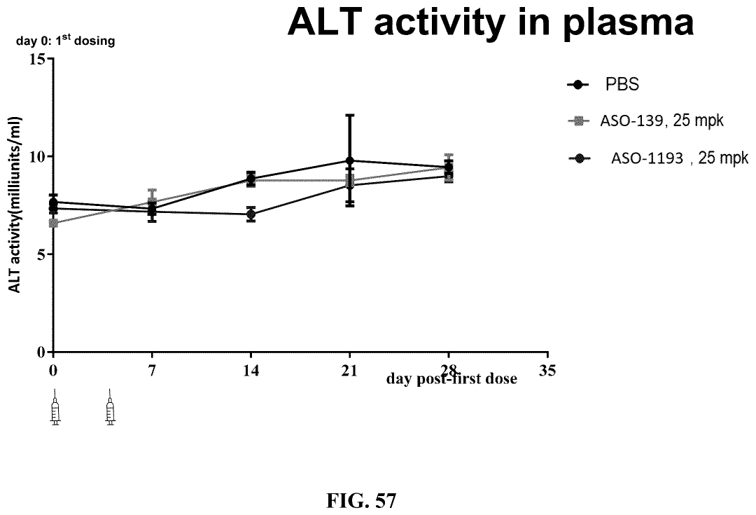

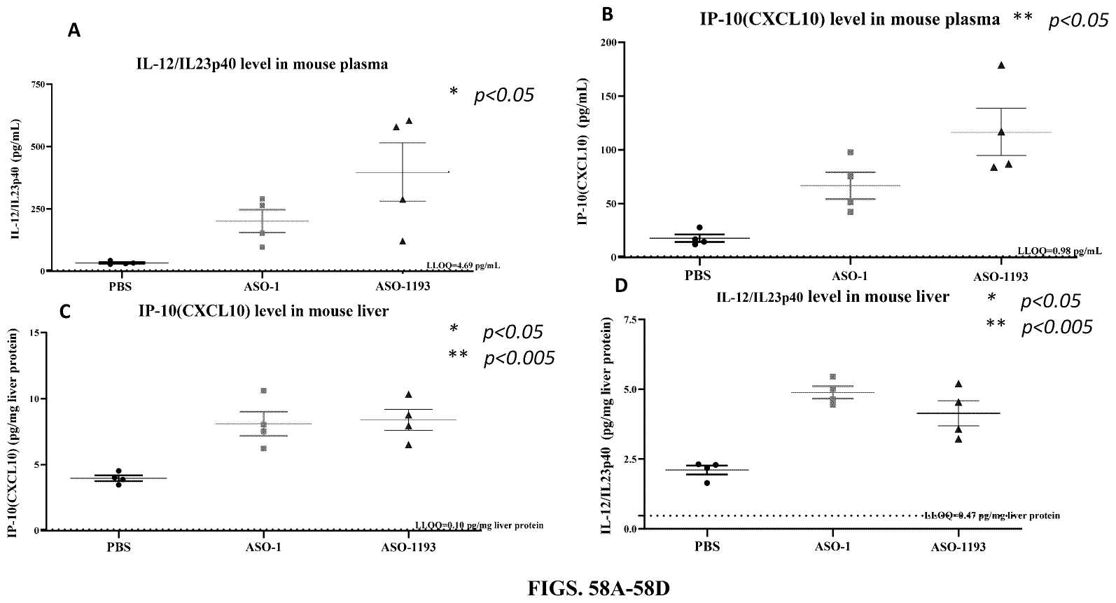

All ASOs show improved caspase profiles compared to ASO-1 and ASO-139 (Table 62 and Table 63). ASO-1191, ASO-1192 and ASO-1193 showed improved RNase H activity as compared to ASO-1 (Table 63). ASO-707, ASO-1191, ASO-1192 and ASO-1193 showed better in vivo potency in AAV-HBV mouse model than ASO-1. ASO-1191, ASO-1192 and ASO-1193 also showed better in vivo potency in AAV-HBV mouse model than ASO-139 (FIG. 45, FIG. 48, FIG. 53, and FIG. 56). ASO-707 showed increased tissue concentration in the liver and improved liver-to-kidney ratio relative to ASO-1 and ASO-139 (FIG. 47). ASO-1191 and ASO-1193 showed similar cytokine induction profiles to ASO-1 (FIG. 55 and FIG. 58).

TABLE 62
Assay Results for ASOs based on ASO-1 with 2′-deoxy
Abasic monomer modification at position 11

| | | Caspase | Caspase | | | | |
|---|---|---|---|---|---|---|---|
| | | Fold | Fold | hTLR8 | | | |
| | | over | over | Fold | hTLR8 | | |
| | | ASO-1 | ASO-1 | over | kD | EC50 | CC50 |
| ASO | Position | 167 nM | 56 nM | ASO-1 | (nM) | (nM) | (nM) |
| ASO-707 | 11 | 0.73 | 0.53 | 0.94 | 0.139 | 19.16 | >167 |
| ASO-1 | N/A | 1 | 1 | 1 | 0.5 | 7.02 | >167 |

TABLE 63
Assay Results for ASOs based on ASO-1 with 2′-deoxy
Abasic monomer modification at position 11 and
LNA modifications at positions 18, 19, or 20

| | | Caspase | Caspase | | | |
|---|---|---|---|---|---|---|
| | | Fold | Fold | | | TLR8 |
| | | over | over | | | Fold |
| | | ASO-1, | ASO-1, | EC50 | CC50 | over |
| ASO | Position | 167 nM | 56 nM | nM | nM | ASO-1 |
| ASO-1 | | 1 | 1 | 8.6 | >167 | 1 |
| ASO-139 | LNA 17, 19, 20 | 1.9 | 1.36 | 1.8 | >167 | 0.8 |
| ASO-1191 | dX 11 LNA 5, 18 | 0.79 | 0.63 | 2.68 | >167 | 1.06 |
| ASO-1192 | dX 11 LNA 18, | 0.59 | 0.61 | 2.83 | >167 | 0.91 |
| | 19 | | | | | |
| ASO-1193 | dX 11 LNA 2, 16 | 0.64 | 0.57 | 2.65 | >167 | 0.91 |

TABLE 64
Assay Results for ASOs based on ASO-
707 with various LNA modifications

| | | Caspase | Caspase | | | hTLR8 |
|---|---|---|---|---|---|---|
| | | Fold | Fold | | | Activity |
| | | over | over | | | Fold |
| Compound | | ASO-1 | ASO-1 | EC50 | CC50 | Over |
| ID | Design | 167 nM | 56 nM | (nM) | (nM) | ASO-1 |
| ASO-1 | | 1 | 1 | 4.02 | >167 | 1.00 |
| ASO-707 | dX11 | 0.78 | 0.88 | 11.67 | >167 | 1.03 |
| ASO-1167 | LNA 3 and 5 | 0.73 | 0.56 | 2.24 | >167 | 1.07 |
| ASO-1168 | LNA 4 and 5 | 0.83 | 0.81 | 5.75 | >167 | 1.09 |
| ASO-1169 | LNA 4 and 16 | 0.73 | 0.67 | 7.88 | >167 | 1.11 |
| ASO-1170 | LNA 5 and 16 | 0.64 | 0.82 | 6.93 | >167 | 1.05 |
| ASO-1171 | LNA 5 and 19 | 0.63 | 0.75 | 4.56 | >167 | 0.93 |
| ASO-1172 | LNA 16 and 18 | 0.30 | 0.00 | 7.36 | >167 | 1.08 |

TABLE 65
Assay Results for ASOs based on ASO-
707 with various LNA modifications

| | | Caspase | Caspase | | | |
|---|---|---|---|---|---|---|
| | | Fold | Fold | | | TLR8 |
| | | over | over | | | Fold |
| Compound | | ASO-1 | ASO-1 | EC50 | CC50 | over |
| ID | Design | 167 nM | 56 nM | (nM) | (nM) | ASO-1 |
| ASO-1 | | 1 | 1 | 5.01 | >167 | 1.00 |
| ASO-139 | | 0.91 | 1.01 | 0.85 | >167 | 0.79 |
| ASO-707 | ASO-1_dX-11 | 0.04 | 0.72 | 14.24 | >167 | 1.03 |
| ASO-1196 | ASO-1dX 11 | 0.79 | 0.47 | 4.74 | >167 | 1.13 |
| | LNA 2 16 | | | | | |
| ASO-1191 | ASO-1 dX 11 | 0.79 | 0.63 | 2.68 | >167 | 1.06 |
| | LNA 5 18 | | | | | |
| ASO-1197 | ASO-1 dX 11 | 0.72 | 0.51 | 2.85 | >167 | 1.03 |
| | LNA 3 18 | | | | | |
| ASO-1198 | ASO-1 dX 11 | 0.01 | 0.62 | 5.22 | >167 | 0.90 |
| | LNA 16 19 | | | | | |
| ASO-1192 | ASO-1 dX 11 | 0.59 | 0.61 | 2.83 | >167 | 0.91 |
| | LNA 18 19 | | | | | |
| ASO-1193 | ASO-1 dX 11 | 0.64 | 0.57 | 2.65 | >167 | 0.81 |
| | LNA 18 20 | | | | | |
| ASO-1199 | ASO-1 dX 11 | 0.75 | 0.54 | 2.88 | >167 | 0.92 |
| | LNA 4 18 | | | | | |

As shown in Table 65, hTLR8 activity in ASOs with two LNAs was retained, while RNaseH mediated activities were improved relative to the parental construct ASO-707. Both ASO-707 and the variants with different LNA patterns displayed better in vitro safety profiles than ASO-1.

### Example 61: ASOs 2′-Deoxy Abasic Monomer in the Gap and LNA Modifications in Varying Positions

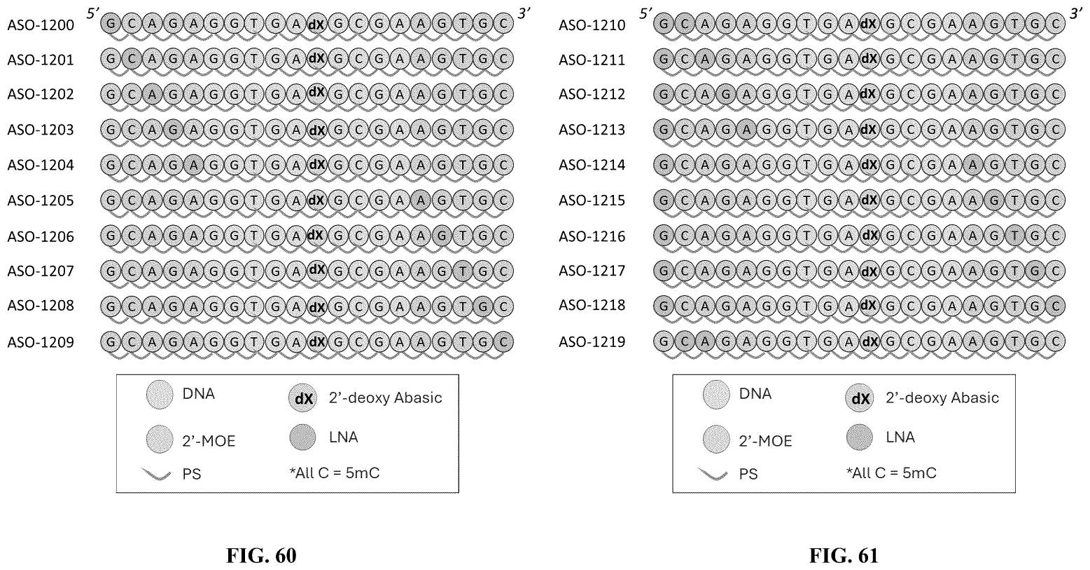

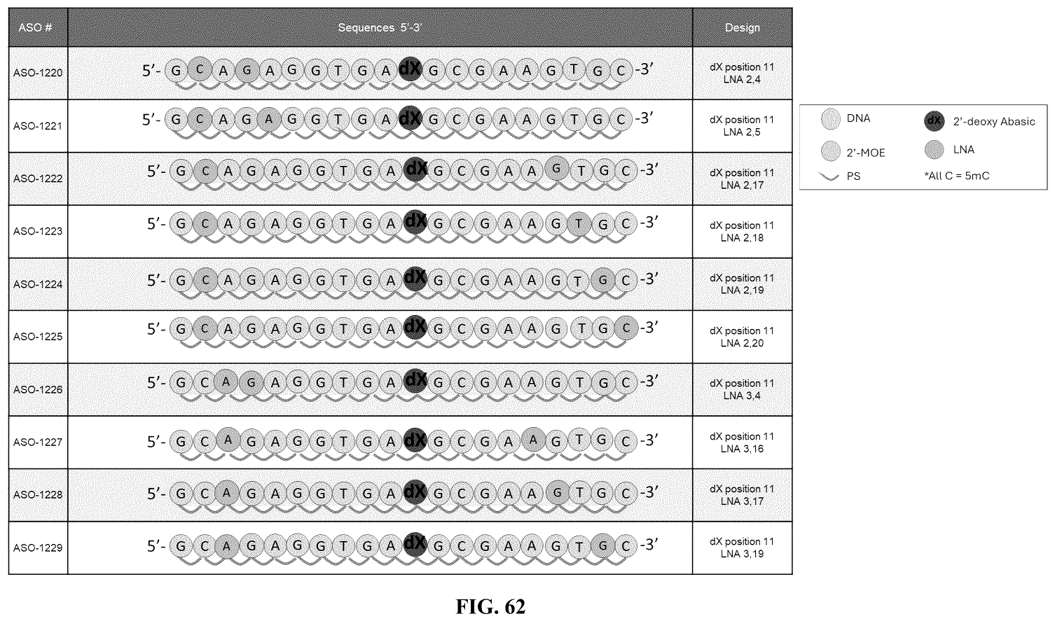

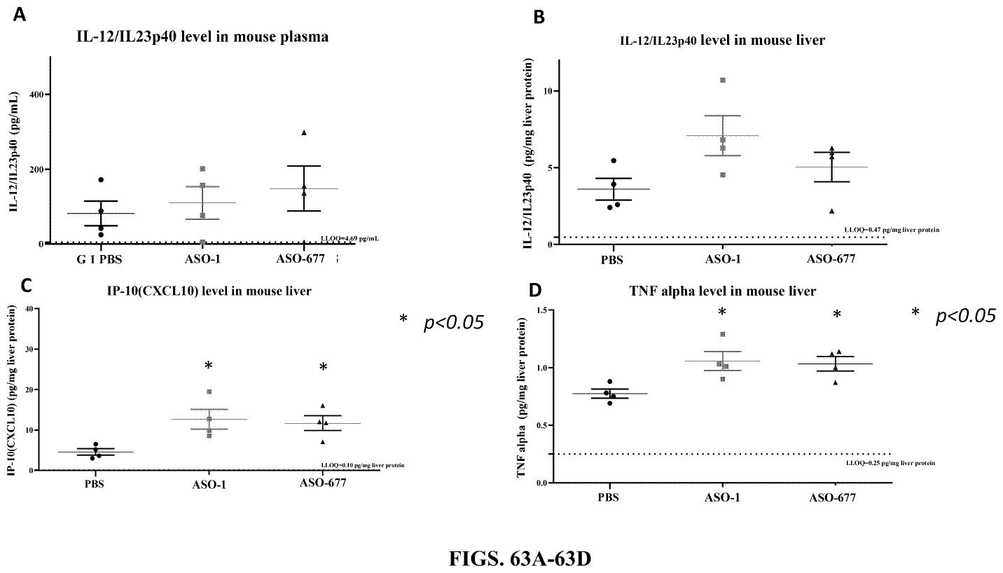

To assess the effect of 2′-deoxy abasic monomers and LNA modifications, ASO-707 was used as the parental ASO and variants were prepared by performing an “LNA walk” to test the impact of one or two LNA nucleotides at various positions (FIGS. 60-62). The ASOs were subjected to the assays described in Example 2 to determine RNAse H-mediated degradation, cytotoxicity, TLR8 pathway activation, and caspase activation as compared to controls ASO-1 and ASO-139 (see Tables 66-68).

TABLE 66
Assay Results for ASOs based on ASO-707 with a 1x LNA walk

| | | Caspase | Caspase | | | |
|---|---|---|---|---|---|---|
| | | Fold over | Fold over | | | TLR8 |
| Compound | | ASO-1 | ASO-1 | EC50 | CC50 | Fold over |
| ID | Design | 167 nM | 56 nM | (nM) | (nM) | ASO-1 |
| ASO-1 | | 1 | 1 | 5.01 | >167 | 1.00 |
| ASO-139 | | 0.91 | 1.01 | 0.85 | >167 | 0.79 |
| ASO-707 | ASO-1_dX-11 | 0.04 | 0.72 | 14.24 | >167 | 1.03 |
| ASO-1200 | dX 11_LNA-Pos1 | 0.98 | 0.86 | 7.44 | >167 | 0.80 |
| ASO-1201 | dX 11_LNA-Pos2 | 1.00 | 0.97 | 4.88 | >167 | 0.90 |
| ASO-1202 | dX 11_LNA-Pos3 | 0.97 | 0.95 | 9.60 | >167 | 0.92 |
| ASO-1203 | dX 11_LNA-Pos4 | 0.95 | 0.91 | 7.11 | >167 | 0.97 |
| ASO-1204 | dX 11_LNA-Pos5 | 0.84 | 1.00 | 8.32 | >167 | 0.86 |
| ASO-1205 | dX 11_LNA-Pos16 | 0.15 | 1.02 | 10.03 | >167 | 0.91 |
| ASO-1206 | dX 11_LNA-Pos17 | 0.11 | 0.95 | 9.68 | >167 | 0.81 |
| ASO-1207 | dX 11_LNA-Pos18 | 0.14 | 0.85 | 4.59 | >167 | 0.97 |
| ASO-1208 | dX 11_LNA-Pos19 | 0.17 | 0.90 | 8.19 | >167 | 0.90 |
| ASO-1209 | dX 11_LNA-Pos20 | 0.14 | 0.96 | 7.41 | >167 | 0.94 |

As shown in Table 66, hTLR8 activity was similar across the 1×LNA walk, but RNaseH mediated activities in the ASOs containing an LNA were improved relative to the parental ASO, ASO-707. Caspase activities for the tested ASOs were similar or reduced relative to ASO-1.

TABLE 67
Assay Results for ASOs based on ASO-707 with a 2x LNA walk

| | | Caspase | Caspase | | | hTLR8 |
|---|---|---|---|---|---|---|
| | | Fold over | Fold over | | | Activity |
| Compound | | ASO-1 | ASO-1 | EC50 | CC50 | Fold Over |
| ID | Design | 167 nM | 56 nM | (nM) | (nM) | ASO-1 |
| ASO-1 | | 1 | 1 | 5.01 | >167 | 1.00 |
| ASO-139 | | 0.91 | 1.01 | 0.85 | >167 | 0.79 |
| ASO-707 | dX-11 | 0.04 | 0.72 | 14.24 | >167 | 1.03 |
| ASO-1210 | dX 11 LNA 12 | 0.78 | 0.47 | 4.26 | >167 | 0.76 |
| ASO-1211 | dX 11 LNA 1 3 | 0.83 | 0.32 | 6.66 | >167 | 0.71 |
| ASO-1212 | dX 11 LNA 1 4 | 0.73 | 0.41 | 6.64 | >167 | 0.77 |
| ASO-1213 | dX 11 LNA 1 5 | 0.82 | 0.75 | 5.3 | >167 | 0.75 |
| ASO-1214 | dX 11 LNA 1 16 | 0.84 | 0.62 | 10.31 | >167 | 0.78 |
| ASO-1215 | dX 11 LNA 1 17 | 0.84 | 0.43 | 8.41 | >167 | 0.87 |
| ASO-1216 | dX 11 LNA 1 18 | 0.81 | 0.42 | 5.28 | >167 | 0.83 |
| ASO-1217 | dX 11 LNA 1 19 | 0.78 | 0.34 | 7.11 | >167 | 0.80 |
| ASO-1218 | dX 11 LNA 1 20 | 0.79 | 0.40 | 6.37 | >167 | 0.85 |
| ASO-1219 | dX 11 LNA 2 3 | 0.95 | 0.35 | 5.46 | >167 | 0.96 |

As shown in Table 67, hTLR8 activity was reduced in most ASOs with two LNAs regardless of position, and improvements in RNaseH activity relative to ASO-707 were observed. Caspase 3/7 activity also improved relative to ASO-1.

TABLE 68
Assay Results for ASOs based on ASO-707 with a 2x LNA walk

| | | Caspase | Caspase | | | |
|---|---|---|---|---|---|---|
| | | Fold over | Fold over | | | TLR8 |
| Compound | | ASO-1 | ASO-1 | EC50 | CC50 | Fold over |
| ID | Design | 167 nM | 56 nM | (nM) | (nM) | ASO-1 |
| ASO-1 | | 1 | 1 | 3.87 | >167 | 1.00 |
| ASO-139 | | 0.91 | 1.01 | 0.99 | >167 | 0.77 |
| ASO-707 | dX-11 | 0.58 | 0.49 | 13.86 | >167 | 0.96 |
| ASO-1220 | dX 11 LNA 2 4 | 0.75 | 1.08 | 6.19 | >167 | 0.90 |
| ASO-1221 | dX 11 LNA 2 5 | 0.98 | 1.46 | 5.56 | >167 | 0.86 |
| ASO-1222 | dX 11 LNA 2 17 | 0.47 | 0.66 | 6.90 | >167 | 0.88 |
| ASO-1223 | dX 11 LNA 2 18 | 0.63 | 1.45 | 4.74 | >167 | 0.67 |
| ASO-1224 | dX 11 LNA 2 19 | 0.61 | 0.60 | 5.69 | >167 | 0.69 |
| ASO-1225 | dX 11 LNA 2 20 | 0.71 | 0.88 | 5.15 | >167 | 0.67 |
| ASO-1226 | dX 11 LNA 3 4 | 0.82 | 0.75 | 7.42 | >167 | 0.82 |
| ASO-1227 | dX 11 LNA 3 16 | 0.72 | 0.91 | 8.58 | >167 | 0.84 |
| ASO-1228 | dX 11 LNA 3 17 | 0.59 | 0.78 | 8.44 | >167 | 0.74 |
| ASO-1229 | dX 11 LNA 3 19 | 0.67 | 0.72 | 5.47 | >167 | 0.75 |

As shown in Table 68, hTLR8 activity was reduced in most ASOs with two LNAs regardless of position, and improvements in RNaseH activity relative to ASO-707 were observed. Caspase 3/7 activity was generally similar to ASO-707 and better than ASO-1.

Activity screening was also performed for ASO-1230, ASO-1231, ASO-1232, ASO-1233, ASO-1234, ASO-1235, ASO-1236, ASO-1237, ASO-1238, and ASO-1239, and similar results were obtained to other ASO variants of ASO-707 containing 2 LNAs. Namely, as shown in Table 69, hTLR8 activity was similar reduced relatively to ASO-707, and similar or reduced RNaseH activity was observed. All of these ASO variants had improved (i.e., reduced) caspase activity, with ASO-1232 having the lowest caspase 3/7 activity.

TABLE 69
Assay Results for ASOs based on ASO-707 with a 2x LNA walk

| | | Caspase | Caspase | | | |
|---|---|---|---|---|---|---|
| | | Fold over | Fold over | | | TLR8 |
| Compound | | ASO-1 | ASO-1 | EC50 | CC50 | Fold over |
| ID | Design | 167 nM | 56 nM | (nM) | (nM) | ASO-1 |
| ASO-1 | | 1 | 1 | 5.05 | >167 | 1.00 |
| ASO-139 | | 0.91 | 1.01 | 0.53 | >167 | 0.76 |
| ASO-707 | dX-11 | 0.74 | 0.35 | 15.50 | >167 | 0.98 |
| ASO-1230 | dX 11 LNA 3 20 | 0.49 | 0.29 | 9.58 | >167 | 0.86 |
| ASO-1231 | dX 11 LNA 4 17 | 0.34 | 0.24 | 21.68 | >167 | 1.17 |
| ASO-1232 | dX 11 LNA 4 19 | 0.15 | 0.22 | 53.48 | >167 | 1.08 |
| ASO-1233 | dX 11 LNA 4 20 | 0.68 | 0.42 | 9.77 | >167 | 0.84 |
| ASO-1234 | dX 11 LNA 5 17 | 0.57 | 0.43 | 10.34 | >167 | 0.81 |
| ASO-1235 | dX 11 LNA 16 20 | 0.43 | 0.31 | 14.95 | >167 | 0.92 |
| ASO-1236 | dX 11 LNA 17 18 | 0.39 | 0.25 | 16.02 | >167 | 0.80 |
| ASO-1237 | dX 11 LNA 17 19 | 0.33 | 0.21 | 17.52 | >167 | 0.77 |
| ASO-1238 | dX 11 LNA 17 20 | 0.40 | 0.33 | 22.69 | >167 | 0.81 |
| ASO-1239 | dX 11 LNA 19 20 | 0.45 | 0.39 | 10.98 | >167 | 0.91 |

### Example 62: Assessment of Conjugated and Unconjugated ASO In Vivo

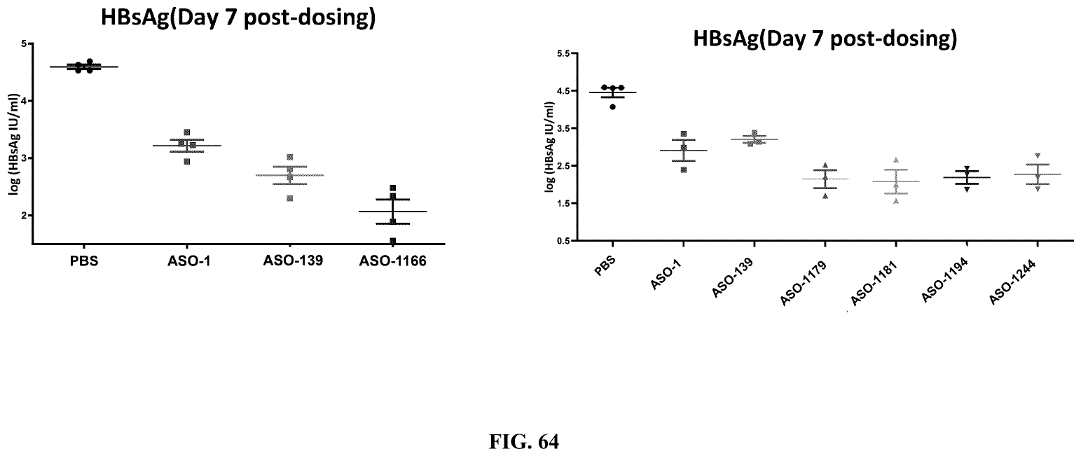

The effect of LNA modified mono-GalNAc conjugated and unconjugated ASOs on HBsAg levels in plasma was evaluated in an AAV-HBV C57BL/6 mouse model essentially as described in Example 25, but only for a 7 day time course. Mice were injected with 25 mg/kg on day 0, and plasma was collected and tested for HBsAg on day 7 (see FIGS. 64-66).

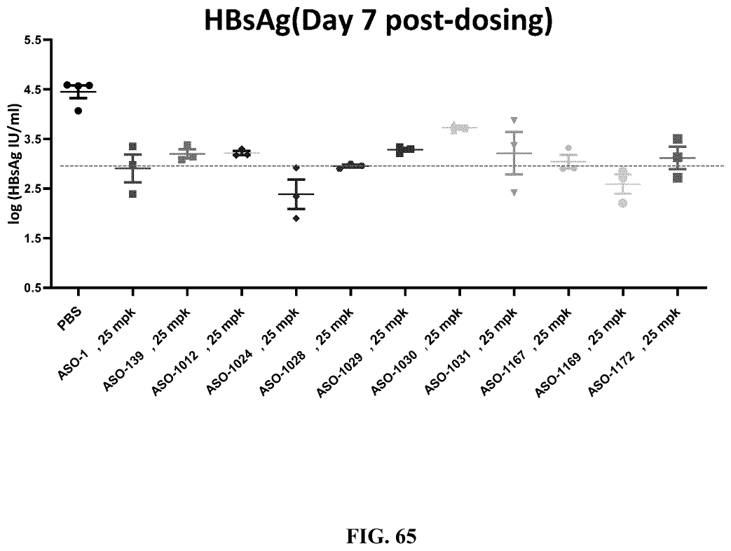

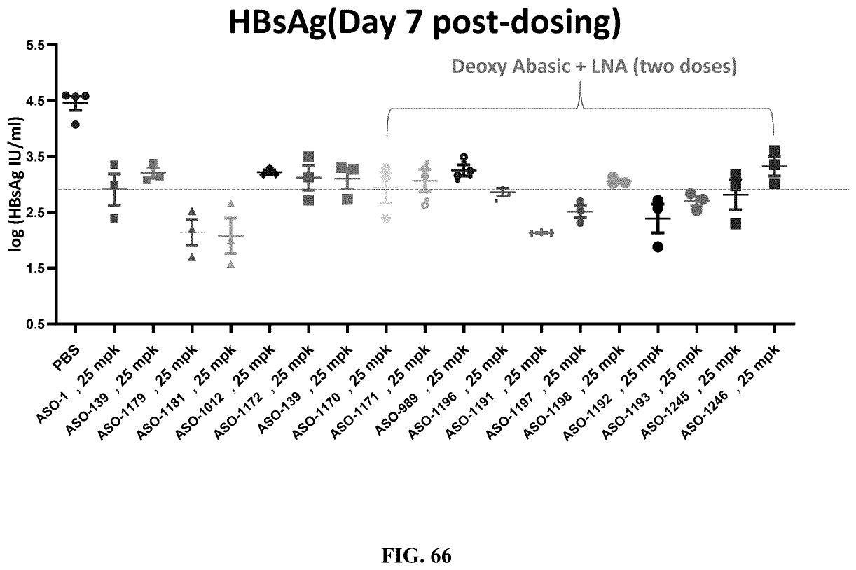

As shown in FIG. 64, ASO-1166, ASO-1179, and ASO-1181 showed significantly improved in vivo potency relative to ASO-1 and ASO-139, thus showing that a mono-GalNAC can improve potency. However, FIG. 65 shows that even without a GalNAc, as ASO-1024 showed modestly improved in vivo potency relative to ASO-1 and ASO-139. ASO-1169 also showed modestly improved in vivo potency relative to ASO-1 and ASO-139. Finally, FIG. 66 shows ASO-1191, ASO-1197, ASO-1192, and ASO-1193 all outperformed the reference ASOs (ASO-1 and ASO-139) despite not containing a GalNAc.

### Example 63: ASOs 2′-Deoxy Abasic Monomer in the Gap and LNA Modifications in Wings

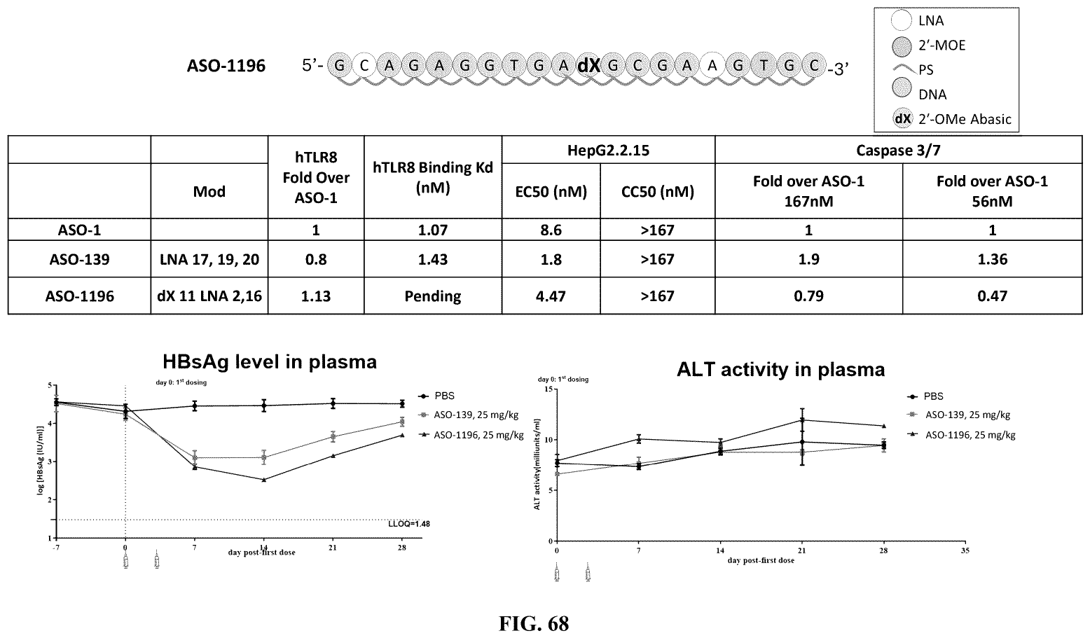

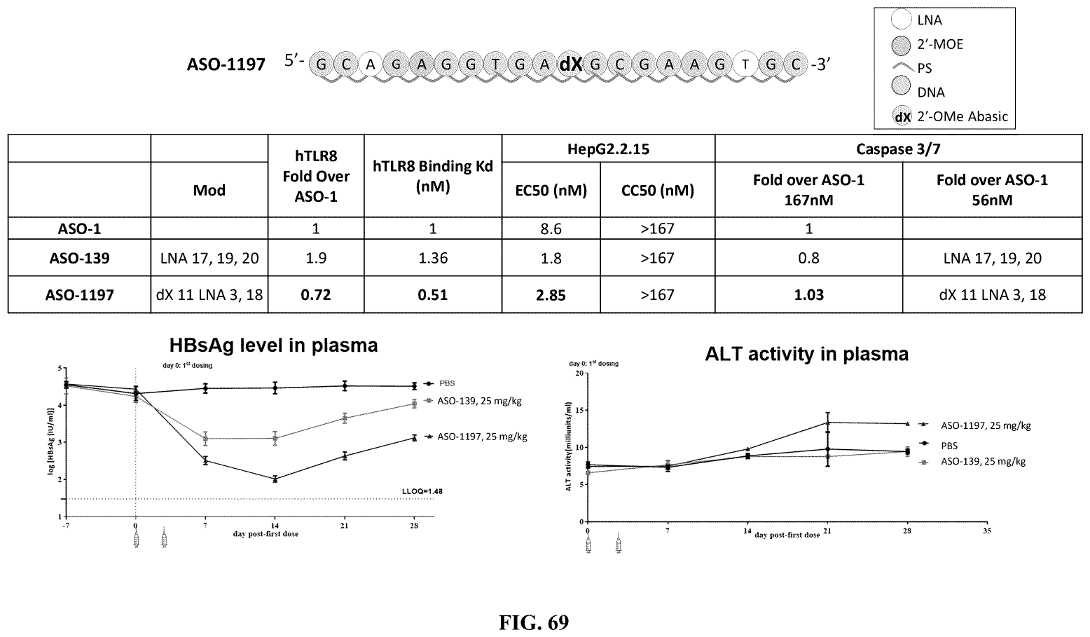

To assess the effect of 2′-deoxy abasic monomers and LNA modifications in each wing, ASO-1196 and ASO-1197 were prepared and subjected to the assays described in Example 2 to determine RNAse H-mediated degradation, cytotoxicity, TLR8 pathway activation, and caspase activation as compared to controls ASO-1 and ASO-139 (see FIGS. 68 and 69).

The effect of ASO-1196 and ASO-1197 on HBsAg and Terminal Human ALT1 was also evaluated in HBV infected PXB mice. Mice were injected with two doses of PBS or two doses of 25 mg/kg per dose of ASO over a time course of 28. While ASO-1196 showed a better overall in vitro profile, ASO-1197 performed better in vivo. Both ASO-1196 and ASO-1197 outperformed ASO-139.

### Example 64: RNase H Activity Assay

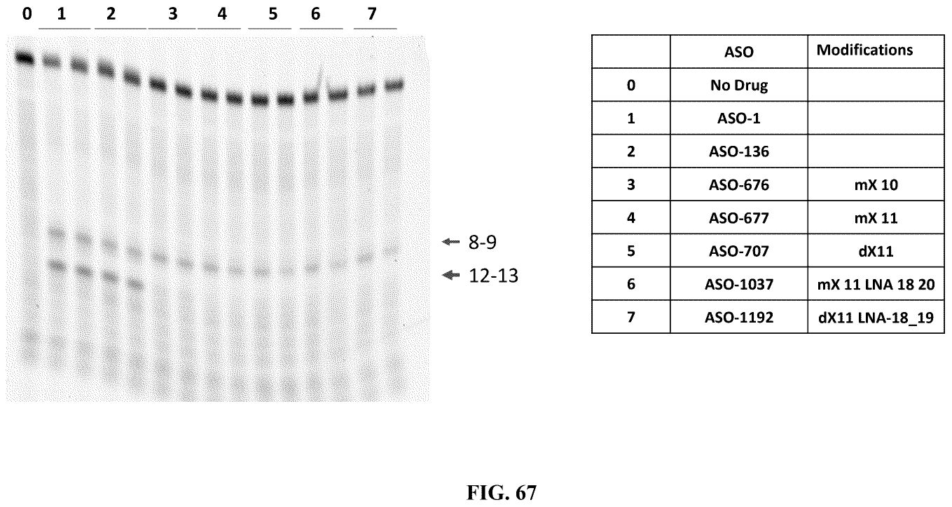

ASOs incorporating abasic monomers in the gap region (including ASO-676, ASO-677, ASO-707, ASO-1037, and ASO-1192) were compared against reference ASOs, ASO-1 and ASO-139, to assess RNase H activity. Human RNase H A1 was expressed in E. coli and purified. The target molecule AS836T, which includes a central RNA sequence flanked by deoxyribonucleotides and a 3′ Cy5, was synthesized by IDT. For the RNase H cleavage reaction, equal molar concentrations of ASO and the target were mixed at 60 nM for annealing, heated to 95° C. for 5 minutes, then cooled to 22° C. over 1 hour, followed by a 7-minute incubation at 22° C. The annealed oligos (2.5 μl) were incubated with RNase H (final concentration 0.06 mg/ml) and RNase inhibitor at 37° C. for 40 minutes in an 8 μl final volume. Reactions were stopped with Formamide sample buffer containing bromophenol blue, heated at 95° C. for 5 minutes,loaded onto a 15% PAGE-Urea gel, and separated at 250 volts for 2.5 hours. Signals from target and cleaved molecules were detected using the Typhoon 8600 Variable Mode Imager and processed with ImageQuant 5.2 software. As shown in FIG. 67, the ASOs incorporating at least one abasic monomer in the gap region showed altered RNase H cleavage.

### Example 65: ASOs 2′-Ome Abasic Monomer in the Gap and LNA Modifications in Wings

Further LNA walks were performed to assess the effect of LNA modifications in the wings of ASOs containing a 2′-OMe abasic monomer in the gap region. Using ASO-676, new ASOs (ASO-1269, -1270, -1271, -1272, -1297, -1298, -1299, -1300, -1301, and -1302) were prepared with an LNA at each different position within the wings. These ASOs were prepared and subjected to the assays described in Example 2 to determine RNAse H-mediated degradation, cytotoxicity, TLR8 pathway activation, and caspase activation as compared to controls ASO-1 and ASO-139 (see Table 70). The results showed that adding one LNA can improve in vitro RNase H activity and maintain a low caspase profile.

TABLE 70
Assay Results for ASOs based on ASO-676 with a 1x LNA walk

| | | | | | Caspase 3/7 | Caspase 3/7 |
|---|---|---|---|---|---|---|
| | | HepG2.2.15 | HepG2.2.15 | hTLR8 | Activity | Activity |
| | | HBsAg | HBsAg | Emax | Over | Over |
| Compound | | EC50 | CC50 | Fold over | ASO-1 | ASO-1 |
| ID | Design | (nM) | (nM) | ASO-1 | @167 nM | @ 56 nM |
| ASO-1 | | 5.81 | >167 | 1.00 | 1 | 1 |
| ASO-132 | | 0.97 | >167 | 0.77 | 0.99 | 1.68 |
| ASO-676 | mX 10 | 9.46 | >167 | 0.91 | 0.18 | 0.005 |
| ASO-1272 | LNA-Pos1 | 7.02 | >167 | 0.71 | 0.49 | 0.31 |
| ASO-1271 | LNA-Pos2 | 6.03 | >167 | 0.81 | 0.60 | 0.05 |
| ASO-1270 | LNA-Pos3 | 7.52 | >167 | 0.82 | 0.62 | 0.25 |
| ASO-1269 | LNA-Pos4 | 8.51 | >167 | 0.82 | 0.41 | 0.12 |
| ASO-1302 | LNA-Pos5 | 8.39 | >167 | 0.99 | 0.42 | 0.10 |
| ASO-1301 | LNA-Pos16 | 13.96 | >167 | 1.08 | 0.28 | 0.07 |
| ASO-1300 | LNA-Pos17 | 7.67 | >167 | 0.96 | −0.08 | −0.35 |
| ASO-1299 | LNA-Pos18 | 5.60 | >167 | 0.96 | 0.79 | 0.50 |
| ASO-1298 | LNA-Pos19 | 6.96 | >167 | 0.95 | 0.82 | 0.53 |
| ASO-1297 | LNA-Pos20 | 5.66 | >167 | 0.95 | 0.60 | 0.31 |

The same experiments were performed using ASO-676 as a parental ASO, but performing a 2×LNA walk in the wings to assess impacts on activity. These ASOs were prepared and subjected to the assays described in Example 2 to determine RNAse H-mediated degradation, cytotoxicity, TLR8 pathway activation, and caspase activation as compared to controls ASO-1 and ASO-139 (see Table 71). The results showed that adding two LNA can improve in vitro RNase H activity and maintain a low caspase profile.

TABLE 71
Assay Results for ASOs based on ASO-676 with a 2x LNA walk

| | | | | | Caspase 3/7 | Caspase 3/7 |
|---|---|---|---|---|---|---|
| | | HepG2.2.15 | HepG2.2.15 | hTLR8 | Activity | Activity |
| | | HBsAg | HBsAg | Emax | Over | Over |
| Compound | | EC50 | CC50 | Fold over | ASO-1 | ASO-1 |
| ID | Design | (nM) | (nM) | ASO-1 | @167 nM | @ 56 nM |
| ASO-1 | | 5.81 | >167 | 1.00 | 1 | 1 |
| ASO-139 | | 0.97 | >167 | 0.77 | 0.99 | 1.68 |
| ASO-676 | mX 10 | 9.46 | >167 | 0.91 | 0.18 | 0.005 |
| ASO-1284 | LNA_Pos2-16 | 9.21 | >167 | 1.10 | 0.56 | 0.17 |
| ASO-1283 | LNA_Pos2-17 | 5.49 | >167 | 0.96 | 0.90 | 0.32 |
| ASO-1282 | LNA_Pos2-18 | 4.53 | >167 | 0.79 | 1.16 | 0.55 |
| ASO-1281 | LNA_Pos2-19 | 4.95 | >167 | 0.82 | 0.85 | 0.29 |
| ASO-1280 | LNA_Pos2-20 | 4.28 | >167 | 0.85 | 0.88 | 0.10 |
| ASO-1279 | LNA-Pos3-4 | 6.93 | >167 | 1.11 | 0.72 | 0.18 |
| ASO-1278 | LNA-Pos3-5 | 6.69 | >167 | 1.02 | 0.84 | 0.25 |
| ASO-1277 | LNA-Pos3-16 | 10.58 | >167 | 1.13 | 0.75 | 0.25 |
| ASO-1276 | LNA-Pos3-17 | 6.32 | >167 | 1.01 | 0.49 | 0.021 |
| ASO-1275 | LNA-Pos3-18 | 5.44 | >167 | 0.92 | 0.63 | 0.024 |
| ASO-1274 | LNA-Pos3-19 | 5.84 | >167 | 0.83 | 0.79 | 0.016 |
| ASO-1273 | LNA-Pos3-20 | 3.50 | >167 | 0.83 | 0.83 | 0.011 |
| ASO-1303 | mX 10 LNA 4 5 | 5.40 | >167 | 0.81 | 0.73 | 0.36 |
| ASO-1304 | mX 10 LNA 4 16 | 10.50 | >167 | 0.86 | 0.35 | 0.04 |
| ASO-1305 | mX 10 LNA 4 17 | 6.93 | >167 | 0.78 | 0.11 | 0.010 |
| ASO-1306 | mX 10 LNA 4 18 | 5.41 | >167 | 0.81 | 0.52 | 0.16 |
| ASO-1307 | mX 10 LNA 4 19 | 4.63 | >167 | 0.78 | 0.57 | 0.00 |
| ASO-1308 | mX 10 LNA 4 20 | 3.50 | >167 | 0.74 | 0.79 | 0.15 |
| ASO-1309 | mX 10 LNA 5 16 | 8.79 | >167 | 0.93 | 0.71 | 0.46 |
| ASO-1310 | mX 10 LNA 5 17 | 7.33 | >167 | 0.81 | 0.76 | 0.61 |
| ASO-1311 | mX 10 LNA 5 18 | 4.37 | >167 | 0.82 | 0.93 | 0.55 |
| ASO-1312 | mX 10 LNA 5 19 | 4.92 | >167 | 0.75 | 0.87 | 0.38 |
| ASO-1313 | mX 10 LNA 5 20 | 3.95 | >167 | 0.72 | 1.00 | 0.79 |
| ASO-1314 | mX 10 LNA 16 17 | 10.70 | >167 | 0.72 | 0.54 | 0.21 |
| ASO-1315 | mX 10 LNA 16 18 | 8.97 | >167 | 1.05 | ND | ND |
| ASO-1316 | mX 10 LNA 16 19 | 9.54 | >167 | 1.00 | ND | ND |
| ASO-1317 | mX 10 LNA 16 20 | 7.62 | >167 | 1.06 | ND | ND |
| ASO-1318 | mX 10 LNA 17 18 | 4.82 | >167 | 0.69 | ND | ND |
| ASO-1319 | mX 10 LNA 17 19 | 5.14 | >167 | 0.75 | ND | ND |
| ASO-1320 | mX 10 LNA 17 20 | 3.46 | >167 | 0.74 | ND | IND |
| ASO-1321 | mX 10 LNA 18 19 | 3.16 | >167 | 0.81 | ND | ND |
| ASO-1322 | mX 10 LNA 18 20 | 2.37 | >167 | 0.68 | ND | ND |
| ASO-1323 | mX 10 LNA 19 20 | 3.84 | >167 | 0.73 | ND | ND |

### Example 66: ASOs 2′-Locked Abasic Monomer (InX) in the Gap

An exploratory walk was performed to assess the effect of incorporating a 2′-locked abasic monomer (InX) in the gap region. New ASOs were prepared with an InX at each different position within the gap. These ASOs were prepared and subjected to the assays described in Example 2 to determine RNAse H-mediated degradation, cytotoxicity, TLR8 pathway activation, and caspase activation as compared to controls ASO-1 and ASO-139 (see Table 72). Interestingly h TLR8 activity was maintained by many ASOs.

TABLE 72
Assay Results for ASOs with lnX in the Gap

| | | | | hTLR8 | Caspase | Caspase |
|---|---|---|---|---|---|---|
| | | | | Activity | Fold Over | Fold Over |
| Compound | | HepG2.2.15 | HepG2.2.15 | Fold over | ASO-1 | ASO-1 |
| ID | Design | EC50 nM | CC50 nM | ASO-1 | @ 167 nM | @ 56 nM |
| ASO-1 | | 5.70 | >167 | 1.00 | 1 | 1 |
| ASO-139 | | 0.98 | >167 | 0.85 | | |
| ASO-1266 | XLNA 6 | 14.11 | >167 | 0.93 | 0.61 | 0.21 |
| ASO-1265 | XLNA 7 | 17.33 | >167 | 0.92 | 0.71 | 0.58 |
| ASO-1264 | XLNA 8 | 16.44 | >167 | 1.08 | 0.70 | 0.33 |
| ASO-1263 | XLNA 9 | 20.1 | >167 | 0.98 | 0.73 | 0.77 |
| ASO-1262 | XLNA 10 | 17.35 | >167 | 1.02 | 0.63 | 0.08 |
| ASO-1261 | XLNA 11 | 13.56 | >167 | 1.07 | 0.95 | 0.29 |
| ASO-1260 | XLNA 12 | 35.01 | >167 | 0.89 | 0.76 | 0.38 |
| ASO-1259 | XLNA 13 | 28.4 | >167 | 1.08 | 0.79 | 0.29 |
| ASO-1268 | XLNA 14 | 28.3 | >167 | 0.95 | 0.70 | 0.21 |
| ASO-1267 | XLNA 15 | 25.35 | >167 | 0.84 | 0.96 | 0.53 |

### Example 67: ASOs 2′-MOE Abasic Monomer (moeX) in the Gap

An exploratory walk was performed to assess the effect of incorporating a 2′-MOE abasic monomer (moeX) in the gap region. New ASOs were prepared with a moeX at each different position within the gap. These ASOs were prepared and subjected to the assays described in Example 2 to determine RNAse H-mediated degradation, cytotoxicity, TLR8 pathway activation, and caspase activation as compared to controls ASO-1 and ASO-139 (see Table 73).

TABLE 73
Assay Results for ASOs with moeX in the Gap

| | | | | hTLR8 | Caspase | Caspase |
|---|---|---|---|---|---|---|
| | | | | Activity | Fold Over | Fold Over |
| Compound | | HepG2.2.15 | HepG2.2.15 | Fold over | ASO-1 | ASO-1 |
| ID | Design | EC50 nM | CC50 nM | ASO-1 | @ 167 nM | @ 56 nM |
| ASO-1 | | 5.70 | >167 | 1.00 | 1 | 1 |
| ASO-139 | | 0.98 | >167 | 0.85 | | |
| ASO-677 | mX 11 | 6.48 | >167 | 0.94 | 0.40 | 0.27 |
| ASO-1249 | moeX-Pos6 | 12.76 | >167 | 0.96 | 0.97 | 0.28 |
| ASO-1250 | moeX-Pos7 | 15.30 | >167 | 1.01 | 0.63 | 0.31 |
| ASO-1251 | moeX-Pos8 | 13.20 | >167 | 0.99 | 0.14 | 0.44 |
| ASO-1252 | moeX-Pos9 | 15.67 | >167 | 0.70 | 0.43 | 0.23 |
| ASO-1253 | moeX-Pos10 | 13.46 | >167 | 0.71 | 0.29 | 0.41 |
| ASO-1254 | moeX-Pos11 | 11.40 | >167 | 0.76 | 0.41 | 0.36 |
| ASO-1255 | moeX-Pos12 | 27.17 | >167 | 0.62 | 0.28 | 0.34 |
| ASO-1256 | moeX-Pos13 | 18.10 | >167 | 0.72 | 0.29 | 0.44 |
| ASO-1257 | moeX-Pos14 | 20.07 | >167 | 0.67 | 0.17 | 0.82 |
| ASO-1258 | moeX-Pos15 | 22.86 | >167 | 0.69 | 0.57 | 0.27 |

### Example 68: Preparation of Abasic Monomer 1

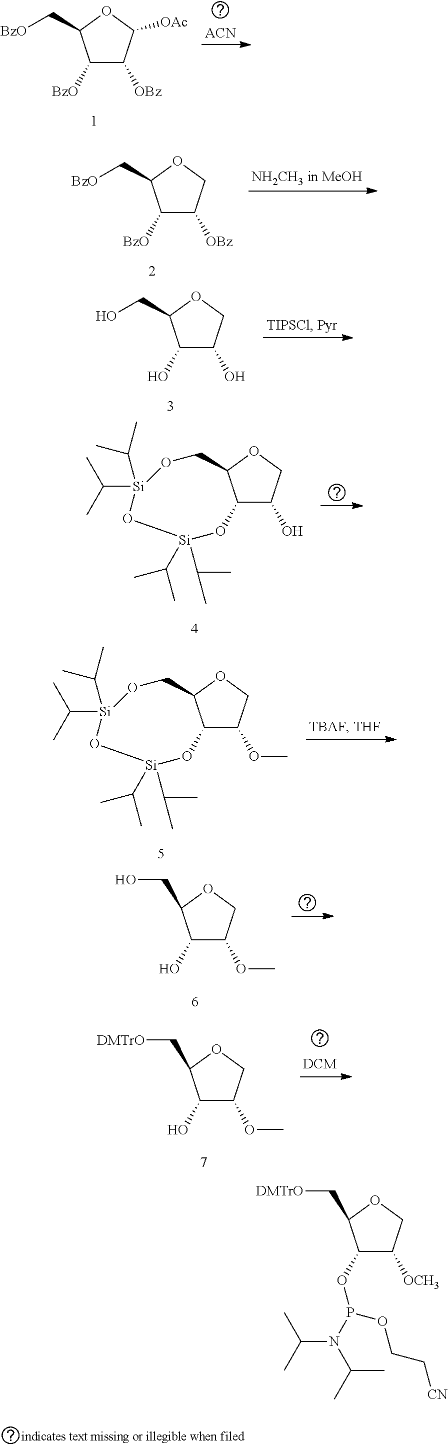

Preparation of (2): To a solution of 1 (360.0 g, 713.6 mmol), Et₃SiH (248.9 g, 2140.8 mmol) in ACN (1080 mL), TMSOTf (317.2 g, 1427.2 mmol) was added at r.t. The mixture was refluxed at 70° C. for 16 h. LC-MS showed 1 was consumed completely. The reaction solution was concentrated under reduced pressure, and then the reaction mixture was added into a solution of NaHCO₃. The mixture was extracted with EA (1.5 L*3). The combined organic layer was washed with brine, dried over Na₂SO₄, filtered and concentrated under reduced pressure. This resulted in crude 2 (343.0 g,) as a brown solid which was used directly for the next step; ESI-LCMS: m/z 446.9 [M+H]⁺.

Preparation of (3): [Journal of the Chemical Society, Perkin transactions 1, 1983, #11, p. 2675-2679]. To a solution of crude 2 (343.0 g, 768.3 mmol) in MeOH (500 mL) was added to a solution of 33% NH₂CH₃ in MeOH (3430 mL). The reaction mixture was stirred at r.t. for 14 h. LC-MS showed 2 was consumed completely. Solvent was removed under reduced pressure, The residue was purified by slurry with a mixture solvent of PE and EA (1:1, 2000 mL) to give 3 (86.0 g, 90.0% yield over two steps from 1 to intermediate 3) as a white solid; ¹H NMR (400 MHZ, Methanol-d₄) δ=4.17-4.11 (m, 1H), 4.05-3.93 (m, 2H), 3.79-3.68 (m, 3H), 3.59-3.54 (m, 1H).

Preparation of (4): To a solution of 3 (86.0 g, 641.2 mmol) in pyridine (860 mL) was added TIPSCI (222.5 g, 705.3 mmol). The reaction mixture was stirred at r.t. for 16 h. TLC showed 3 was consumed completely. The reaction solution was quenched with water (1.0 L), and the product was extracted with EA (1.0 L*3). The combined organic layer was washed with brine, dried over Na₂SO₄, filtered and concentrated under reduced pressure. The residue was purified by silica gel column chromatograph (eluent, PE:EA=50:1˜10:1) to give 4 (204.0 g, 84.5% yield) as a colorless oil; ¹H NMR (400 MHZ, DMSO-d₆) δ=4.70 (d, J=3.7 Hz, 1H, exchanged with D₂O), 4.11-4.00 (m, 2H), 3.96-3.92 (m, 1H), 3.91-3.79 (m, 2H), 3.66-3.62 (m, 1H), 3.56-3.52 (m, 1H), 1.03 (m, 28H).

Preparation of (5): To a solution of 4 (16.0 g, 42.5 mmol) in Mel (80 mL) was added Ag₂O (39.4 g, 169.9 mmol). The reaction mixture was refluxed at 40° C. for 16 h. TLC showed about 60% of 4 was consumed. Then mixture was filtered, the filtrates were concentrated under reduced pressure and purified by silica gel column chromatography (eluent, PE:EA=60:1˜50:1) to give 5 (7.0 g, 42.0% yield) as a colorless oil; ¹H NMR (400 MHZ, DMSO-d₆) δ=3.99-3.97 (m, 1H), 3.91-3.87 (m, 1H), 3.85-3.80 (m, 2H), 3.65-3.59 (m, 2H), 3.43 (s, 3H), 1.08-0.99 (m, 28H).

Preparation of (6): [Carbohydrate Research, 1995, vol. 274, #1, p. 99-110]. To a solution of 5 (7.0 g, 17.9 mmol) in THF (140 mL) was added TBAF (21.5 mL, 21.5 mmol). The reaction mixture was stirred at r.t for 8 h. TLC showed 5 was consumed completely. Solvent was removed under reduced pressure. This resulted in to give crude 6 (11.5 g, 77.6 mmol) as a yellow oil which was used directly for the next step.

Preparation of (7): To a solution of crude 6 (11.5 g, 77.6 mmol) in pyridine (115 mL) was added DMTrCl (78.9 g, 232.9 mmol). The reaction mixture was stirred at r.t. for 14 h. TLC showed 6 was consumed completely. The reaction solution was quenched with water (200 mL), and the product was extracted with EA (200.0 mL*3). The combined organic layer was washed with brine, dried over Na₂SO₄, filtered and concentrated under reduced pressure. The residue was purified by silica gel column chromatograph (eluent, PE:EA=10:1˜3:1) to give 7 (4.8 g, 59.8% yield over two steps from intermediate 5 to intermediate 7) as a yellow oil; ESI-LCMS: m/z 449.2 [M−H]⁺; ¹H NMR (400 MHZ, DMSO-d₆) δ=7.42-7.37 (m, 2H), 7.32-7.28 (m, 2H), 7.27-7.22 (m, 4H), 7.22-7.17 (m, 1H), 6.90-6.87 (m, 4H), 4.79 (d, J=7.0 Hz, 1H, exchanged with D₂O), 3.95-3.85 (m, 2H), 3.74 (s, 6H), 3.73-3.69 (m, 2H), 3.70-3.68 (m, 1H), 3.33 (s, 3H), 3.10-3.07 (m, 1H), 2.95-3.91 (m, 1H).

Preparation of (a protected version of Abasic Monomer 1): To a solution of 5 (2.3 g, 5.1 mmol) in DCM (23 mL) with an inert atmosphere of nitrogen was added CEOP[N(iPr)₂]₂ (1.7 g, 5.6 mmol) and DCI (0.4 g, 3.6 mmol). The resulting solution was concentrated under reduced pressure. The residue was purified by Flash-Prep-HPLC with the following conditions (IntelFlash-1): Column, C18 silica gel; mobile phase, CH: CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Abasic Monomer 1 (2.6 g, 78.2% yield) as a colorless oil; ESI-LCMS: m/z 651.2 [M+H]⁺; ¹H NMR (400 MHZ, DMSO-d6) δ=7.43-7.38 (m, 2H), 7.34-7.16 (m, 7H), 6.93-6.84 (m, 4H), 4.27-4.08 (m, 1H), 4.03-3.82 (m, 3H), 3.82-3.67 (m, 8H), 3.60-3.40 (m, 3H), 3.38-3.31 (m, 3H), 3.27-3.10 (m, 1H), 3.00-2.90 (m, 1H), 2.80-2.70 (m, 1H), 2.60-2.52 (m, 1H), 1.15-1.02 (m, 9H), 0.97-0.87 (m, 3H). ³¹P NMR (162 MHz, DMSO-d6) δ=148.603, 148.464.

### Example 69: Preparation of Abasic Monomer 3

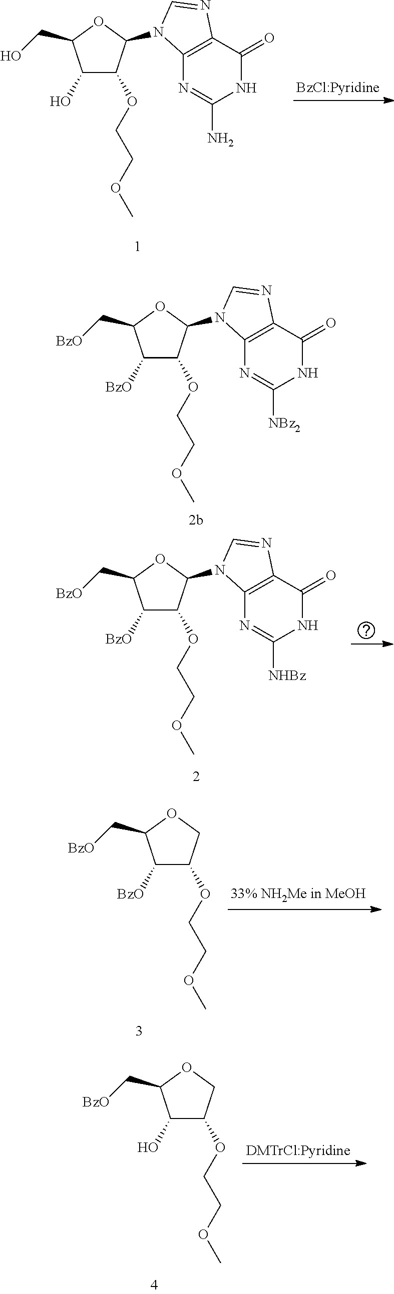

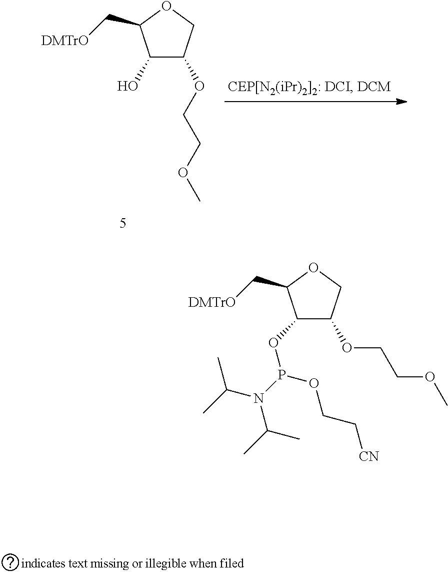

Preparation of (2): To a solution of 1 (30.0 g, 87.9 mmol) in pyridine (300 mL) was added BzCl (49.4 g, 351.6 mmol). The mixture was stirred at r.t. for 8 hours. LC-MS showed 1 was consumed completely. Then diluted in EA and washed with saturated aqueous sodium bicarbonate twice followed by brine, then dried over Na₂SO₄, filtered and concentrated under reduced pressure. This resulted in crude 2 and 2b (65.0 g, the ratio of 2 and 2b=1:1), which was used directly for the next step.

Preparation of (3): To a solution of crude 2 and 2b (65.0 g) in ACN (280 mL) was added Et₃SiH (33.8 g, 290.7 mmol) and dropped TMSOTf (64.6 g, 290.1 mmol) into the reaction. The reaction mixture was stirred at 60° C. for 40 h. LC-MS showed 2 was consumed completely. The combined organic layer was washed with water and brine, dried over Na₂SO₄, and concentrated under reduced pressure. The residue was purified by silica gel column chromatograph (eluent, PE:EA=20:1˜5:1). This resulted in 3 (11.0 g, 31.2% yield over two steps from 1 to intermediate 3) as a colorless oil; ESI-LCMS: m/z 401.1 [M+H]⁺; ¹H NMR (400 MHZ, CDCl₃-d) δ=8.10-8.02 (m, 4H), 7.58-7.55 (m, 2H), 7.53-7.39 (m, 4H), 5.39-5.36 (m, 1H), 4.59-4.54 (m, 1H), 4.50-4.46 (m, 2H), 4.36-4.32 (m, 1H), 4.26-4.23 (m, 1H), 3.98-3.94 (m, 1H), 3.70-3.64 (m, 2H), 3.45-3.42 (m, 2H), 3.24 (s, 3H).

Preparation of (4): A solution of 3 (11.0 g, 27.4 mmol) in 33% of CH₃NH₂ methanol (110 mL) was stirred at r.t for 4 hours. TLC (DCM:MeOH=10:1) showed 3 was consumed completely. The solution was concentrated under reduced pressure to get crude 4 (3.0 g), which was used directly for the next step.

Preparation of (5): To a solution of crude 4 (3.0 g) in pyridine (30 mL) was added DMTrCl (5.8 g, 17.2 mmol). The mixture was stirred at r.t. for 4 hours. TLC (DCM:MeOH=10:1) showed 3 was consumed completely. Then dissolved in EA and washed with saturated aqueous sodium bicarbonate twice followed by brine, then dried over Na₂SO₄, filtered and concentrated under reduced pressure. The residue was purified by silica gel column chromatograph (eluent, PE:EA=100:1˜40:1) to get product 5 (3.5 g, 25.9% yield over two steps from intermediate 3 to intermediate 5). ESI-LCMS: m/z 493.2 [M−H]⁻; ¹H NMR (400 MHZ, CDCl₃-d) δ=7.47-7.41 (m, 1H), 7.37-7.23 (m, 6H), 7.21-7.13 (m, 2H), 6.89-6.76 (m, 4H), 4.19-4.12 (m, 1H), 4.12-4.04 (m, 2H), 4.01-3.93 (m, 1H), 3.85-3.81 (m, 3H), 3.79 (s, 6H), 3.70-3.64 (m, 1H), 3.62-3.48 (m, 2H), 3.40 (d, J=3.2 Hz, 3H), 3.31-3.25 (m, 1H), 3.11 (m, 1H).

Preparation of (a protected version of Abasic monomer 3): To a solution of 5 (3.5 g, 7.1 mmol) in DCM (35 mL) was added DCI (0.5 g, 5.7 mmol) and CEP[N(iPr)₂]₂ (2.75 g, 10.1 mmol) at r.t at N₂. The mixture was stirred at r.t. for 2 h. LC-MS showed 5 was consumed completely. The solution was washed with water twice and washed with brine and dried over Na₂SO₄, filtered. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Abasic monomer 3 (3.6 g, 73.2% yield) as a white solid; ESI-LCMS: m/z 617.2 [M+H]⁺; ¹H NMR (400 MHZ, DMSO-d₆) δ=7.43-7.12 (m, 9H), 6.94-6.80 (m, 4H), 4.15-4.12 (m, 1H), 4.04-3.87 (m, 3H), 3.79-3.41 (m, 15H), 3.25 (d, J=4.7 Hz, 3H), 3.22-3.13 (m, 1H), 2.96-2.93 (m, 1H), 2.78-2.71 (m, 1H), 2.54 (d, J=6.5 Hz, 1H), 1.14-1.04 (m, 8H), 0.91 (d, J=6.8 Hz, 4H); ³¹P NMR (162 MHz, DMSO-d₆) δ=148.40, 148.12.

### Example 70: Preparation of Abasic Monomer 2

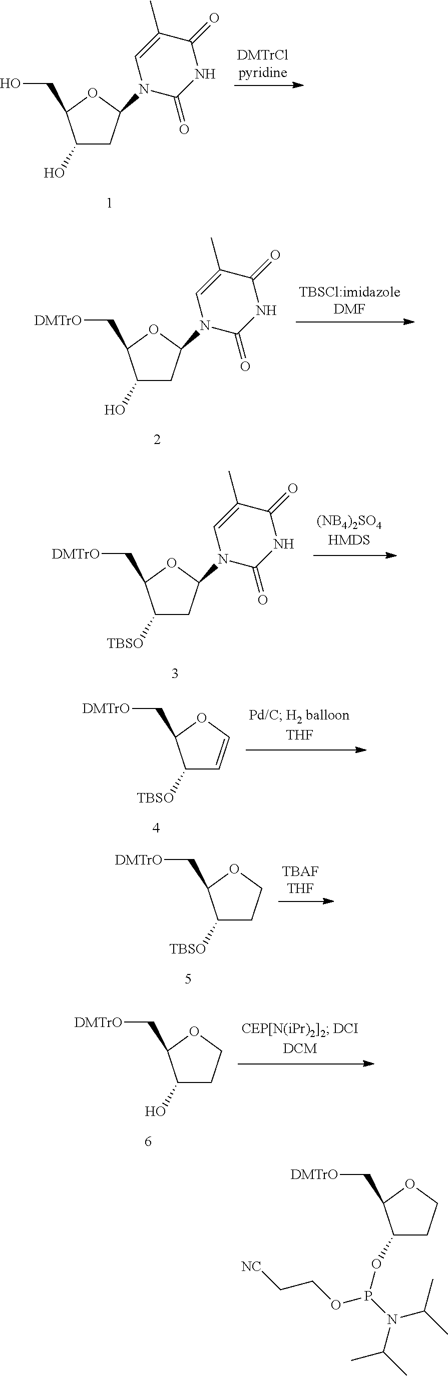

Preparation of (2): To a solution of 1 (200.0 g, 825.7 mmol) in pyridine (2.0 L) was added DMTrCl (307.3 g, 909.1 mmol) at r.t at N₂. The solution was stirred at r.t for 1 hours. The solution was diluted with EA (10.0 L), washed with water (2×15.0 L) and brine (5.0 L). The organic phase was dried over anhydrous Na₂SO₄, concentrated by rotary evaporator to give crude 2 (460.0 g) as a yellow oil which was used directly for the next step. ESI-LCMS m/z 543.2 [M−H].⁻

Preparation of (3): To a solution of crude 2 (460.0 g) in DMF (4.0 L) was added imidazole (281.0 g, 4.1 mol) and TBSCI (250.0 g, 1.6 mol) at r.t at N₂. The solution was stirred at r.t for 16 hours. The solution was diluted with EA (10.0 L), washed with water (2×15.0 L) and brine (4.0 L). The organic phase was dried over anhydrous Na₂SO₄, then the solution was concentrated under reduced pressure. The residue was isolated by silica gel column chromatography (eluent, PE/EA=20:1). This resulted in to give 3 (440.0 g, 80.9% yield over two steps from 1 to intermediate 3) as a white solid. ESI-LCMS m/z 657.2 [M−H].⁻

Preparation of (4): To a solution of 3 (440.0 g, 667.8 mmol) in HMDS (4.0 L) was added (NH₄)₂SO₄ (313.1 g, 2.3 mol) at 135° C. at N₂. The solution was stirred at 135° C. for 16 hours. TLC show SM was completely consumed. The solution was diluted with EA (10.0 L), washed with water (2×10.0 L) and brine (5.0 L). The organic phase was dried over anhydrous Na₂SO₄, then the solution was concentrated under reduced pressure. The residue was isolated by silica gel column chromatography (eluent, PE/EA=5:1) to give 4 (120.0 g, 225.2 mmol, 33.7% yield) as a yellow oil. ¹H NMR (400 MHZ, DMSO-d₆) δ:7.47-7.20 (m, 9H), 7.00-6.83 (m, 4H), 6.75-6.67 (m, 1H), 5.14-5.04 (m, 1H), 4.88-4.77 (m, 1H), 4.29-4.18 (m, 1H), 3.75 (s, 6H), 3.14-2.99 (m, 2H), 0.829 (s, 9H), 0.00 (s, 6H).

Preparation of (5): To a solution of 4 (100.0 g, 187.7 mmol) in THF (2000 mL) was added 10% of Pd/C (20 g) at r.t under H₂ balloon. The solution was stirred at r.t for 16 hours. TLC show SM was completely consumed. The solution was filtered and the filter was concentrated to give the crude 5 (90.0 g) as a clear oil without further purified and used directly for the next step.

Preparation of (6): [Nucleosides, nucleotides and nucleic acids, 2016, vol. 35, #2, p. 64 75]. To a solution of 5 (90.0 g) in THF (900 mL) was added 1M TBAF in THF (219 mL, 219.1 mmol) at r.t. The solution was stirred at r.t for 15 hours. LCMS and TLC show SM was completely consumed. The solution was deal with EA (1.0 L), washed with water (2×2.0 L) and brine (1.0 L). The organic phase was dried over anhydrous Na₂SO₄, then the solution was concentrated under reduced pressure. The residue was purified by silica gel column chromatograph (eluent, PE/EA=2:1). This resulted in to give 6 (50.5 g, 64.2% yield over two steps from intermediate 4 to intermediate 6) as a yellow oil. ¹H NMR (400 MHz, DMSO-d₆) δ:7.47-7.12 (m, 9H), 6.97-6.81 (m, 4H), 4.94 (d, J=4.4 Hz, 1H), 4.10-3.77 (m, 4H), 3.73 (s, 6H), 3.01-2.85 (m, 2H), 1.98-1.87 (m, 1H), 1.77-1.65 (m, 1H).

Preparation of (a protected version of Abasic monomer 2): To a solution of 6 (50.0 g, 118.9 mmol) in DCM (500 mL) was added DCI (11.9 g, 101.1 mmol) and CEP[N(iPr)₂]₂ (43.0 g, 142.8 mmol) at r.t at N₂. The mixture was stirred at r.t 2 for 2 h. The mixture was added NaHCO₃ aqueous (500 mL) and extracted with DCM (200 mL). Then the organic layer was washed with water (300 mL) and brine (300 mL) and dried over Na₂SO₄. The solution was filtered and the filter was concentrated to give the crude. The crude was purified by Flash-Prep-HPLC with the following conditions (IntelFlash-1): Column, C18 silica gel; mobile phase, CH₃CN/H₂O (0.05% NH₄HCO₃)=3/2 increasing to CH₃CN/H₂O (0.05% NH₄HCO₃)=1/0 within 20 min, the eluted product was collected at CH₃CN/H₂O (0.05% NH₄HCO₃)=9/1; Detector, UV 254 nm. This resulted in to give Abasic monomer 2 (51.0 g, 69.1% yield) as a yellow oil. ESI-LCMS 621.2 m/z [M+H]⁺. ¹H NMR (400 MHZ, DMSO-d₆) δ:7.44-7.16 (m, 9H), 6.97-6.82 (m, 4H), 4.38-4.24 (m, 1H), 3.97-3.76 (m, 3H), 3.76-3.44 (m, 10H), 3.08-2.92 (m, 2H), 2.76-2.70 (m, 1H), 2.67-2.60 (m, 1H), 2.11-1.81 (m, 2H), 1.18-0.93 (m, 12H). ³¹P NMR (162 MHZ, DMSO-d₆) δ 146.81, 146.61.

### Example 71: Preparation of Abasic Monomer 4

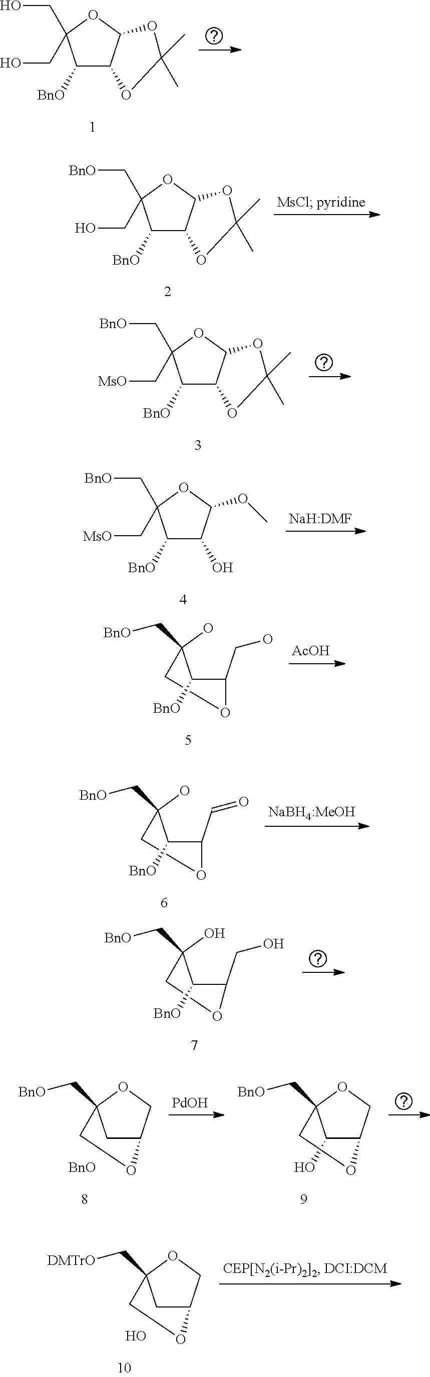

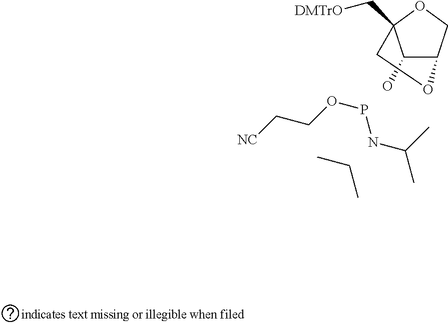

Preparation of (2): To a solution of 1 (50.0 g, 161.1 mmol) in DMF (500 mL) was added NaH (7.1 g, 177.2 mmol, 60% purity) at an ice salt batch to control the internal reaction to 0° C. The mixture was stirred at 0° C. for 1 h, then BnBr (30.3 g, 177.2 mmol) was added drop wise, the mixture was stirred at r.t. for 8 h, TLC showed 1 was consumed completely. The solution was poured into water (2.0 L) and the product was extracted with EA (200 mL*3). The combined organic layer was washed with water and brine, dried over Na₂SO₄, filtered and concentrated under reduced pressure. This resulted in to give crude 2 (57.5 g) as a colorless oil, which was used directly for the next step.

Preparation of (3): To a solution of 2 (57.5 g) in pyridine (600 mL) was added drop wise MsCl (20.3 g, 177.2 mmol) at an ice salt batch to control the internal reaction to 0° C. The reaction mixture was stirred at r.t. for 2 h. TLC showed 2 was consumed completely. The solution was poured into water (2.0 L) and the product was extracted with EA (200 mL*3). The combined organic layer was washed with water and brine, dried over Na₂SO₄, filtered and concentrated under reduced pressure. The residue was purified by silica gel column chromatograph (eluent, PE:EA=10:1˜5:1). This resulted in to give 3 (36.8 g, 47.7% yield over two step from 1 to intermediate 3) as a colorless oil; ¹H NMR (400 MHZ, CDCl₃-d): δ=7.35-7.24 (m, 10H), 5.77 (d, J=3.6 Hz, 1H), 4.85 (d, J=11.4 Hz, 1H), 4.72 (d, J=12.0 Hz, 1H), 4.64-4.62 (m, 1H), 4.54-4.42 (m, 4H), 4.29-4.27 (m, 1H), 3.60-3.58 (m, 1H), 3.52-3.47 (m, 1H), 3.06 (s, 3H,), 1.68 (s, 3H), 1.34 (s, 3H).

Preparation of (4): To a solution of 3 (36.8 g, 76.9 mmol) in MeOH (1500 mL) was added con.aq.HCl (225 mL). The mixture was stirred at r.t. for 16 h. TLC showed 3 was consumed completely. The reaction solution was quenched with water (4.0 L), and the product was extracted with EA (500 mL*3). The combined organic layer was washed with water and brine, dried over Na₂SO₄, filtered and concentrated under reduced pressure give crude 4 (33.8 g) as a colorless oil, which was used directly for the next step.

Preparation of (5): To a solution of 4 (33.8 g,) in DMF (350 mL) was added NaH (3.0 g, 74.6 mmol, 60%) at an ice salt batch to control the internal reaction to 0° C. The mixture was stirred at r.t. for 2 h, TLC showed 4 was consumed completely. The solution was poured into water (2.0 L) and the product was extracted with EA (200 mL*3). The combined organic layer was washed with water and brine, dried over Na₂SO₄, filtered and concentrated under reduced pressure. The residue was purified by silica gel column chromatograph (eluent, PE:EA=1:0˜10:1). This resulted in 5 (17.2 g, 62.8% yield over two steps from intermediate 3 to intermediate 5) as a colorless oil; 1H NMR (600 MHZ, CDCl₃-d): δ=7.31-7.23 (m, 10H), 4.80 (s, 1H), 4.66-4.53 (m, 4H), 4.12-4.08 (m, 2H), 3.99 (d, J=7.2 Hz 1H), 3.8-4.75 (m, 3H), 3.38 (s, 3H).

Preparation of (6): A solution of 5 (17.0 g, 47.7 mmol) in AcOH (170 mL) was stirred at r.t. for 14 h. TLC showed 5 was consumed completely. The solution was poured into water (2.0 L), the product was extracted with EA (0.5 L*3), the combined organic layer washed with brine and dried over Na₂SO₄, filtered. Then the solution was filtered and concentrated under reduced pressure to give crude 4 (16.0 g) as a colorless oil, which was used in the next step directly.

Preparation of (7): To a solution of 6 (16.0 g) in MeOH (160 mL) was added NaBH4 (2.5 g, 91.1 mmol). The mixture was stirred at r.t. for 1 h. TLC showed 6 was consumed completely. The solution was poured into water (1.0 L) and the product was extracted with EA (200 mL*3), the combined organic layer was washed and washed with brine and dried over Na₂SO₄, filtered. Then the solution was concentrated under reduced pressure and the residue was purified by silica gel column chromatograph (eluent, PE:EA=1:0˜15:1). This resulted in 7 (12.8 g, 78.0% yield over two steps from intermediate 5 to intermediate 7) as a colorless oil; 1H NMR (400 MHZ, CDCl₃-d): δ=7.34-7.23 (m, 10H), 4.60-4.53 (m, 4H), 4.03-4.01 (m, 1H), 3.86-3.72 (m, 5H), 3.60-4.57 (m, 2H), 3.15 (s, 2H).

Preparation of (8): [Tetrahedron Letters, 2000, vol. 41, #46, p. 8923-8927]. To a solution of 6 (12.8 g, 37.2 mmol) in THF (130 mL) was added Bu₃P (11.3 g, 55.8 mmol), and DIAD (11.3 g, 55.8 mmol). The reaction mixture was stirred at r.t. for 8 h under N₂. TLC showed 7 was consumed completely. The solution was poured into water (500 mL) and the product was extracted with EA (100 mL*3), the combined organic layer was washed and washed with brine and dried over Na₂SO₄, filtered. Then the solution was concentrated under reduced pressure. The residue was purified by silica gel column chromatograph (eluent, PE:EA=20:1˜5:1). This resulted in 8 (7.5 g, 61.8% yield) as a colorless oil. ¹H NMR (600 MHZ, CDCl₃-d) δ=7.33-7.24 (m, 10H), 4.69-4.56 (m, 4H), 4.29 (s, 1H), 4.06-3.93 (m, 5H), 3.73-3.70 (m, 2H).

Preparation of (9): To a solution of 8 (7.5 g, 23.0 mmol) in MeOH (75 mL) was added Pd/(OH)₂ (1.5 g) under H₂ balloon at r.t for 18 hours. TLC showed 7 was consumed completely. The solids were filtered out and the solution was concentrated the solvent under reduced pressure. This resulted in crude 9 (1.5 g), which was used for the next step directly.

Preparation of (10): To a solution of crude 9 (1.5 g) in pyridine (15 mL) was added DMTrCl (4.2 g, 12.3 mmol). The reaction mixture was stirred at r.t. for 4 h. LC-MS showed 9 was consumed completely. Then the solution was concentrated under reduced pressure and the residue was purified by silica gel column chromatograph (eluent, PE:EA=50:1˜3:1). This resulted in 10 (3.0 g, 29.1% yield over two steps from intermediate 8 to intermediate 10) as a white solid. ¹H NMR (600 MHZ, DMSO-d6) δ=7.28-7.14 (m, 9H), 6.91-6.87 (m, 4H), 5.42 (d, J=4.8 Hz 1H, exchanged with D₂O), 4.07-3.85 (m, 5H), 3.76-3.73 (m, 7H), 3.26-3.14 (m, 2H).

Preparation of abasic monomer 4): [Journal of Organic Chemistry, 2000, vol. 65, #17, p. 5167-5176]. To a solution of 10 (2.5 g, 5.6 mmol) in DCM (25 mL) was added DCI (0.5 g, 4.1 mmol) and CEP[N(iPr)₂]₂ (2.4 g, 8.0 mmol) at r.t under N₂. The mixture was stirred at r.t. for 2 h. LC-MS showed 10 was consumed completely. The solution was washed with water twice and washed with brine and dried over Na₂SO₄, filtered. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give abasic monomer 4 (2.3 g, 66.1% yield) as a white solid; ESI-LCMS: m/z 650.2 [M+H]⁺; ¹H NMR (400 MHZ, DMSO-d₆) δ=7.42-7.20 (m, 9H), 6.99-6.86 (m, 4H), 4.31 (s, 1H), 4.22-4.15 (m, 1H), 4.02-3.96 (m, 2H), 3.86-3.65 (m, 9H), 3.53-3.49 (m, 3H), 3.25-3.17 (m, 1H), 2.79-2.2.75 (m, 1H), 2.62-2.56 (m, 1H), 1.12-1.01 (m, 9H), 0.92-0.87 (m, 3H); ³¹P NMR (162 MHz, DMSO-d6) δ=147.56, 146.58.

### Example 72: Effect of GalNAc on Liver/Kidney Ratio and In Vivo Efficacy

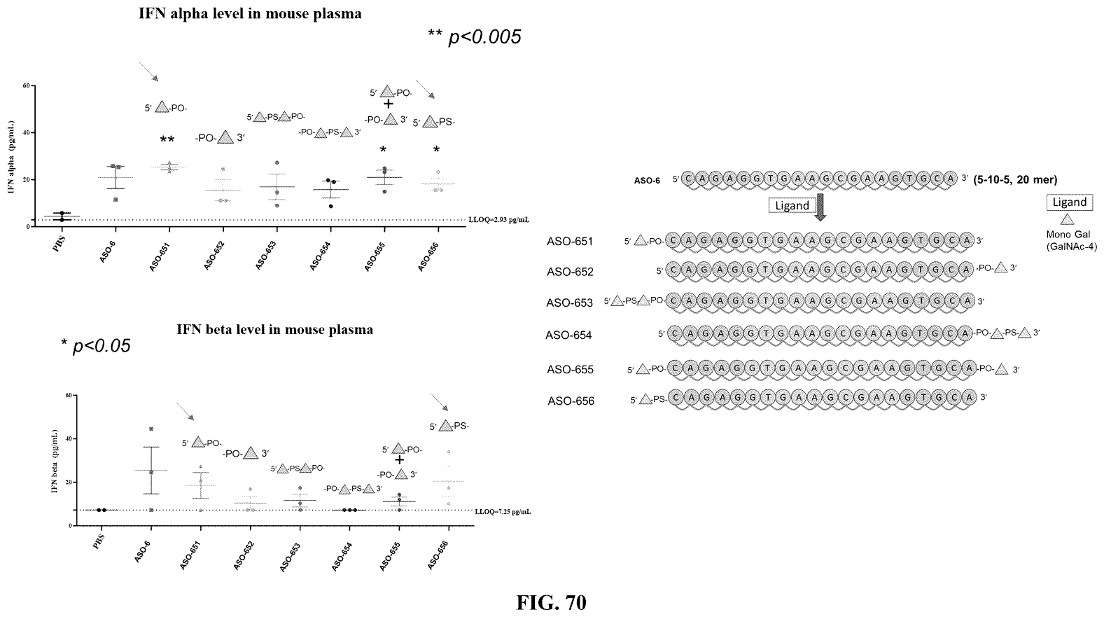

Various GalNAc conjugates were prepared and tested to assess the impact of incorporating a monomeric or dimeric GalNAc (specifically GalNAc 4) onto the end(s) of the disclosed ASOs. Using ASO-6 as a parental ASO, various monomeric or dimeric GalNAcs were conjugates to the ends of the ASO (see FIG. 70) and administered to mice with a hTLRb knock-in. Interferon-α and Interferon-β levels were assessed. As shown in FIG. 70, monomeric GalNAc strikes a favorable balance between RNaseH (shown in other figures) and immune response, thus suggesting monomeric GalNAc may be useful for conjugation onto the disclosed ASOs to treat HBV.

### VI. HBV Nucleotide Sequence

Hepatitis B virus (Genbank Accession No. U95551.1; SEQ ID NO: 1)

(SEQ ID NO: 1)
AATTCCACAACCTTTCACCAAACTCTGCAAGATCCCAGAGTGAGAGGCCTGTATTTC
CCTGCTGGTGGCTCCAGTTCAGGAGCAGTAAACCCTGTTCCGACTACTGCCTCTCCC
TTATCGTCAATCTTCTCGAGGATTGGGGACCCTGCGCTGAACATGGAGAACATCACA
TCAGGATTCCTAGGACCCCTTCTCGTGTTACAGGCGGGGTTTTTCTTGTTGACAAGA
ATCCTCACAATACCGCAGAGTCTAGACTCGTGGTGGACTTCTCTCAATTTTCTAGGG
GGAACTACCGTGTGTCTTGGCCAAAATTCGCAGTCCCCAACCTCCAATCACTCACCA
ACCTCCTGTCCTCCAACTTGTCCTGGTTATCGCTGGATGTGTCTGCGGCGTTTTATCA
TCTTCCTCTTCATCCTGCTGCTATGCCTCATCTTCTTGTTGGTTCTTCTGGACTATCAA
GGTATGTTGCCCGTTTGTCCTCTAATTCCAGGATCCTCAACCACCAGCACGGGACCA
TGCCGAACCTGCATGACTACTGCTCAAGGAACCTCTATGTATCCCTCCTGTTGCTGTA
CCAAACCTTCGGACGGAAATTGCACCTGTATTCCCATCCCATCATCCTGGGCTTTCG
GAAAATTCCTATGGGAGTGGGCCTCAGCCCGTTTCTCCTGGCTCAGTTTACTAGTGC
CATTTGTTCAGTGGTTCGTAGGGCTTTCCCCCACTGTTTGGCTTTCAGTTATATGGAT
GATGTGGTATTGGGGGCCAAGTCTGTACAGCATCTTGAGTCCCTTTTTACCGCTGTTA
CCAATTTTCTTTTGTCTTTGGGTATACATTTAAACCCTAACAAAACAAAGAGATGGG
GTTACTCTCTGAATTTTATGGGTTATGTCATTGGAAGTTATGGGTCCTTGCCACAAGA
ACACATCATACAAAAAATCAAAGAATGTTTTAGAAAACTTCCTATTAACAGGCCTAT
TGATTGGAAAGTATGTCAACGAATTGTGGGTCTTTTGGGTTTTGCTGCCCCATTTACA
CAATGTGGTTATCCTGCGTTAATGCCCTTGTATGCATGTATTCAATCTAAGCAGGCTT
TCACTTTCTCGCCAACTTACAAGGCCTTTCTGTGTAAACAATACCTGAACCTTTACCC
CGTTGCCCGGCAACGGCCAGGTCTGTGCCAAGTGTTTGCTGACGCAACCCCCACTGG
CTGGGGCTTGGTCATGGGCCATCAGCGCGTGCGTGGAACCTTTTCGGCTCCTCTGCC
GATCCATACTGCGGAACTCCTAGCCGCTTGTTTTGCTCGCAGCAGGTCTGGAGCAAA
CATTATCGGGACTGATAACTCTGTTGTCCTCTCCCGCAAATATACATCGTATCCATGG
CTGCTAGGCTGTGCTGCCAACTGGATCCTGCGCGGGACGTCCTTTGTTTACGTCCCGT
CGGCGCTGAATCCTGCGGACGACCCTTCTCGGGGTCGCTTGGGACTCTCTCGTCCCC
TTCTCCGTCTGCCGTTCCGACCGACCACGGGGCGCACCTCTCTTTACGCGGACTCCC
CGTCTGTGCCTTCTCATCTGCCGGACCGTGTGCACTTCGCTTCACCTCTGCACGTCGC
ATGGAGACCACCGTGAACGCCCACCGAATGTTGCCCAAGGTCTTACATAAGAGGAC
TCTTGGACTCTCTGCAATGTCAACGACCGACCTTGAGGCATACTTCAAAGACTGTTT
GTTTAAAGACTGGGAGGAGTTGGGGGAGGAGATTAGATTAAAGGTCTTTGTACTAG
GAGGCTGTAGGCATAAATTGGTCTGCGCACCAGCACCATGCAACTTTTTCACCTCTG
CCTAATCATCTCTTGTTCATGTCCTACTGTTCAAGCCTCCAAGCTGTGCCTTGGGTGG
CTTTGGGGCATGGACATCGACCCTTATAAAGAATTTGGAGCTACTGTGGAGTTACTC
TCGTTTTTGCCTTCTGACTTCTTTCCTTCAGTACGAGATCTTCTAGATACCGCCTCAG
CTCTGTATCGGGAAGCCTTAGAGTCTCCTGAGCATTGTTCACCTCACCATACTGCACT
CAGGCAAGCAATTCTTTGCTGGGGGGAACTAATGACTCTAGCTACCTGGGTGGGTGT
TAATTTGGAAGATCCAGCATCTAGAGACCTAGTAGTCAGTTATGTCAACACTAATAT
GGGCCTAAAGTTCAGGCAACTCTTGTGGTTTCACATTTCTTGTCTCACTTTTGGAAGA
GAAACCGTTATAGAGTATTTGGTGTCTTTCGGAGTGTGGATTCGCACTCCTCCAGCTT
ATAGACCACCAAATGCCCCTATCCTATCAACACTTCCGGAAACTACTGTTGTTAGAC
GACGAGGCAGGTCCCCTAGAAGAAGAACTCCCTCGCCTCGCAGACGAAGGTCTCAA
TCGCCGCGTCGCAGAAGATCTCAATCTCGGGAACCTCAATGTTAGTATTCCTTGGAC
TCATAAGGTGGGGAACTTTACTGGTCTTTATTCTTCTACTGTACCTGTCTTTAATCCT
CATTGGAAAACACCATCTTTTCCTAATATACATTTACACCAAGACATTATCAAAAAA
TGTGAACAGTTTGTAGGCCCACTTACAGTTAATGAGAAAAGAAGATTGCAATTGATT
ATGCCTGCTAGGTTTTATCCAAAGGTTACCAAATATTTACCATTGGATAAGGGTATT
AAACCTTATTATCCAGAACATCTAGTTAATCATTACTTCCAAACTAGACACTATTTAC
ACACTCTATGGAAGGCGGGTATATTATATAAGAGAGAAACAACACATAGCGCCTCA
TTTTGTGGGTCACCATATTCTTGGGAACAAGATCTACAGCATGGGGCAGAATCTTTC
CACCAGCAATCCTCTGGGATTCTTTCCCGACCACCAGTTGGATCCAGCCTTCAGAGC
AAACACAGCAAATCCAGATTGGGACTTCAATCCCAACAAGGACACCTGGCCAGACG
CCAACAAGGTAGGAGCTGGAGCATTCGGGCTGGGTTTCACCCCACCGCACGGAGGC
CTTTTGGGGTGGAGCCCTCAGGCTCAGGGCATACTACAAACTTTGCCAGCAAATCCG
CCTCCTGCCTCCACCAATCGCCAGACAGGAAGGCAGCCTACCCCGCTGTCTCCACCT
TTGAGAAACACTCATCCTCAGGCCATGCAGTGG

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art.

Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds, compositions or biological systems, which can, of course, vary. It is also to be understood that the terminology used herein is for the purpose of describing particular embodiments only and is not intended to be limiting.

All patents, patent applications, provisional applications, and publications referred to or cited herein are incorporated by reference in their entirety, including all figures and tables, to the extent that are not inconsistent with the explicit teachings of this specification.

1. An antisense oligonucleotide (ASO) that is complementary to at least 5 contiguous nucleotides within positions 1570-1610 of SEQ ID NO: 1, has a nucleic acid sequence comprising or consisting of 18-23 nucleotides and, optionally, at least one of the nucleotides is replaced with an abasic monomer, and comprises at least one phosphorothioate linkage and at least one 2′-O-methoxyethyl (2′-MOE) nucleotide.

2-3. (canceled)

4. The ASO of claim 1, wherein the nucleic acid sequence comprises any one of SEQ ID NOs: 321-352.

5-7. (canceled)

8. The ASO of claim 1, wherein the ASO comprises:
(a) a 5′-wing region (A′) comprising 2 to 7 locked nucleotides or substituted nucleotides;
(b) a central region (B′) comprising 5 or more contiguous nucleotides, wherein the central region comprises DNA nucleotides; and
(c) a 3′-wing region (C′) comprising 2 to 7 locked nucleotides or substituted nucleotides.

9-14. (canceled)

15. The ASO of claim 8, wherein the central region (B′) comprises at least one RNA: at least one 2′-substituted nucleotide selected from 2′-O-cyp, 2′-O-mcyp, 2′-OMe, 2′-OMe-3′-xylo; at least one nucleotide with a modified base selected from (8nh)G, (8nh)A, (2s)T, and (5oh)C; at least one abasic monomer; or any combination thereof.

16. The ASO of claim 15, wherein the ASO comprises an abasic monomer selected from abasic monomer 1, abasic monomer 2, abasic monomer 3, and abasic monomer 4 located at position 1, 2, 3, 4, 5, 6, 7, 8, 9, or 10 of the central region (B′) relative to the 5′ end of the ASO.

17-22. (canceled)

23. An antisense oligonucleotide (ASO) comprising any one of SEQ ID NOs: 3-320, 353-404, and 446-1344.

24. (canceled)

25. A pharmaceutical composition comprising the ASO according to claim 1 and a pharmaceutically acceptable excipient.

26. A method of treating a subject having a Hepatitis B virus (HBV) infection, comprising administering to the subject with HBV an ASO according to claim 1.

27-35. (canceled)

36. An antisense oligonucleotide (ASO), comprising:
(a) a 5′-wing region (A′) comprising 2 to 7 nucleotides;
(b) a central region (B′) comprising up to 16 positions comprising at least one abasic monomer and 5 to 15 nucleotides; and
(c) a 3′-wing region (C′) comprising 2 to 7 nucleotides.

37. The ASO according to claim 36, wherein the 2 to 7 nucleotides of the 5′-wing region (A′) are locked nucleotides or substituted nucleotides, and wherein the 2 to 7 nucleotides of the 3′-wing region (C′) are locked nucleotides or substituted nucleotides.

38. (canceled)

39. The ASO according to claim 36, wherein the at least one abasic monomer has a structure of 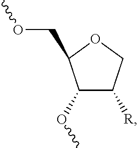
wherein R is H, an alkyl, an alkoxy, O-cyp, or O-mcyp or R can connect to the 4′ of the sugar to form a locked abasic monomer, and wherein [symbol] represents a phosphodiester linkage, a phosphorothioate linkage, or a mesyl phosphoroamidate linkage.

40. The ASO according to claim 36, wherein the at least one abasic monomer is selected from abasic monomer 1, abasic monomer 2, abasic monomer 3, and abasic monomer 4.

41. The ASO according to claim 36, wherein the central region (B′) comprises 10 positions and the at least one abasic monomer is located at any one of positions 4, 5, or 6 of the central region (B′).

42. The ASO according to claim 36, wherein the 5′-wing region (A′) and the 3′-wing region (C′) each independently comprise 5 nucleotides, and wherein the 5 nucleotides comprise locked nucleotides, 2′-MOE nucleotides, or a combination thereof.

43. (canceled)

44. The ASO according to claim 36, wherein
(i) the 5 to 15 nucleotides of the central region (B′) are DNA;
(ii) at least one and up to all linkages in the ASO are phosphorothioate linkages;
(iii) the central region (B′) contains only one abasic monomer; or
(iv) any combination of (i)-(iii).

45-47. (canceled)

48. The ASO according to claim 36, wherein:
the 5′-wing region (A′) and the 3′-wing region (C′) each independently comprise five 2′-MOE nucleotides, the central region (B′) comprises 10 positions and the at least one abasic monomer is located at any one of positions 4, 5, or 6 of the central region (B′), wherein all linkages in the ASO are phosphorothioate linkages; and, optionally, wherein:
(i) the 5′-wing region (A′) comprises one or two locked nucleic acids (LNAs);
(ii) the 3′-wing region (C′) comprises one or two LNAs; or
(iii) the 5′-wing region (A′) comprises one LNA and the 3′-wing region (C′) comprises one LNA.

49. The ASO according to claim 36, wherein:
(a) the 5′-wing region (A′) and the 3′-wing region (C′) each independently comprise five 2′-MOE nucleotides, the central region (B′) comprises 10 positions consisting of one abasic monomer 1 at position 4 and the remaining positions being DNA nucleotides, wherein all linkages in the ASO are phosphorothioate linkages;
(b) the 5′-wing region (A′) and the 3′-wing region (C′) each independently comprise five 2′-MOE nucleotides, the central region (B′) comprises 10 positions consisting of one abasic monomer 1 at position 5 and the remaining positions being DNA nucleotides, wherein all linkages in the ASO are phosphorothioate linkages;
(c) the 5′-wing region (A′) and the 3′-wing region (C′) each independently comprise five 2′-MOE nucleotides, the central region (B′) comprises 10 positions consisting of one abasic monomer 1 at position 6 and the remaining positions being DNA nucleotides, wherein all linkages in the ASO are phosphorothioate linkages;
(d) the 5′-wing region (A′) and the 3′-wing region (C′) each independently comprise five 2′-MOE nucleotides, the central region (B′) comprises 10 positions consisting of one abasic monomer 2 at position 4 and the remaining positions being DNA nucleotides, wherein all linkages in the ASO are phosphorothioate linkages;
(e) the 5′-wing region (A′) and the 3′-wing region (C′) each independently comprise five 2′-MOE nucleotides, the central region (B′) comprises 10 positions consisting of one abasic monomer 2 at position 5 and the remaining positions being DNA nucleotides, wherein all linkages in the ASO are phosphorothioate linkages;
(f) the 5′-wing region (A′) and the 3′-wing region (C′) each independently comprise five 2′-MOE nucleotides, the central region (B′) comprises 10 positions consisting of one abasic monomer 2 at position 6 and the remaining positions being DNA nucleotides, wherein all linkages in the ASO are phosphorothioate linkages;
(g) the 5′-wing region (A′) comprises five 2′-MOE nucleotides; the 3′-wing region (C′) comprises five positions comprising 2′-MOE nucleotides at positions 1, 2, and 4 and LNAs at positions 3 and 5; and the central region (B′) comprises 10 positions consisting of one abasic monomer 1 at position 6 and the remaining positions being DNA nucleotides, wherein all linkages in the ASO are phosphorothioate linkages; or
(h) the 5′-wing region (A′) comprises five positions comprising 2′-MOE nucleotides at positions 1, 2, 4, and 5 and an LNAs at position 3; the 3′-wing region (C′) comprises five positions comprising 2′-MOE nucleotides at positions 1, 2, 4, and 5 and an LNA at position 3; the central region (B′) comprises 10 positions consisting of one abasic monomer 2 at position 6 and the remaining positions being DNA nucleotides, wherein all linkages in the ASO are phosphorothioate linkages.

50. The ASO according to claim 36, wherein the ASO is selected from ASO-676 (SEQ ID NO: 697), ASO-677 (SEQ ID NO: 698), ASO-1037 (SEQ ID NO: 1058), ASO-707 (SEQ ID NO: 728), ASO-1192 (SEQ ID NO: 1213), ASO-962 (SEQ ID NO: 983), ASO-678 (SEQ ID NO: 699), ASO-1191 (SEQ ID NO: 1212), ASO-1193 (SEQ ID NO: 1213), ASO-1036 (SEQ ID NO: 1057), ASO-651 (SEQ ID NO: 672), ASO-1166 (SEQ ID NO: 1187), ASO-1181 (SEQ ID NO: 1202), and ASO-1179 (SEQ ID NO: 1200).

51. The ASO according to claim 36, wherein the ASO further comprises a conjugate attached to the 5′ end or 3′ end of the ASO, wherein the conjugate comprises a GalNAc.

52. (canceled)

53. The ASO according to claim 51, wherein the GalNAc is a monomeric GalNAc.

54. The ASO according to claim 51, wherein the GalNAc is GalNAc 4.

55. (canceled)
